(19) United States
(12) Patent Application Publication
(10) Pub. No.: US 2024/0368114 A1
(43) Pub. Date: Nov. 7, 2024

(54) INDAZOLES, PHARMACEUTICAL COMPOSITIONS, AND THERAPEUTIC APPLICATIONS

(71) Applicant: BioTheryX, Inc., San Diego, CA (US)

(72) Inventors: Leah M. Fung, San Diego, CA (US); Shenlin Huang, San Diego, CA (US); Venkat Reddy Mali, San Diego, CA (US); Nataraj Pagadala, Murrieta, CA (US)

(21) Appl. No.: 18/626,823

(22) Filed: Apr. 4, 2024

Related U.S. Application Data
(60) Provisional application No. 63/494,460, filed on Apr. 6, 2023.

(51) Int. Cl. C07D 401/04; A61K 31/454; A61K 31/4545; A61K 31/496; A61K 31/497; A61K 31/501; A61K 31/5025; A61K 31/506; A61K 31/5365; A61K 31/5377; A61K 31/538; A61K 31/541; A61K 31/55; C07D 405/14; C07D 409/14; C07D 413/14; C07D 417/14; C07D 471/04; C07D 487/04; C07D 495/04; C07D 498/04; C07D 513/04

(52) U.S. Cl. CPC C07D 401/04; A61K 31/454; A61K 31/4545; A61K 31/496; A61K 31/497; A61K 31/501; A61K 31/5025; A61K 31/506; A61K 31/5365; A61K 31/5377; A61K 31/538; A61K 31/541; A61K 31/55; C07D 405/14; C07D 409/14; C07D 413/14; C07D 417/14; C07D 471/04; C07D 487/04; C07D 495/04; C07D 498/04; C07D 513/04

(57) ABSTRACT

Provided herein are indazoles, e.g., a compound of Formula (I), and pharmaceutical compositions thereof. Also provided herein are methods of their use for treating, preventing, or ameliorating one or more symptoms of a disorder, disease, or condition mediated by a zinc-figure protein, casein kinase 1α, GSPT1, or PDE6D.

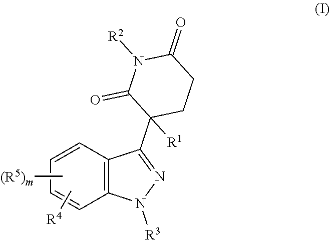

### CROSS REFERENCE TO RELATED APPLICATION

This application claims the benefit of U.S. Provisional Application No. 63/494,460, filed Apr. 6, 2023; the disclosure of which is incorporated herein by reference in its entirety.

### FIELD

Provided herein are indazoles and pharmaceutical compositions thereof. Also provided herein are methods of their use for treating, preventing, or ameliorating one or more symptoms of a disorder, disease, or condition mediated by a zinc-figure protein, casein kinase 1α, GSPT1, or PDE6D.

### BACKGROUND

Molecular glues are monovalent compounds that bind simultaneously two distinct proteins, e.g., a ubiquitin ligase and a target protein, leading to the ubiquitination and subsequent proteasome-based degradation of the target protein. Schreiber Cell 2020, 184, 3-9; Kozicka and Thoma, Cell Chem. Biol. 2021, 28, 1032-47; Faust et al., Ann. Rev. Cancer Biol. 2021, 5, 181-201. Revlimid (lenalidomide), one of the most successful anticancer drugs, is a molecular glue. Kronke et al., Science 2014, 343, 301-5. Molecular glues have several advantages over traditional small-molecule enzyme inhibitors or receptor antagonists. Dong et al., J. Med. Chem. 2021, 64, 10606-20. Mechanistically, molecular glues drive target ubiquitination and degradation in a substoichiometric and catalytic manner. Id. As they do not require a druggable pocket on the target protein, molecular glues are able to degrade a therapeutic target that is intractable by conventional approaches. Id.

Molecular glues are distinctly different from proteolysis targeting chimeras (PROTACs). While PROTACs are bifunctional molecules-chimeras having two moieties connected together by a linker, molecular glues are monovalent small molecules that act as adhesives to bring two proteins together. These linker-based bifunctional PROTACs generally have high molecular weight (MW), poor cellular permeability, and unfavorable pharmacokinetic (PK) profiles. Edmondson et al., Bioorg. Med. Chem. Lett. 2019, 29, 1555-64. The physicochemical properties of molecular glues are similar to those of traditional small-molecule drugs. Thus, molecular glues generally possess more favorable drug-like properties, such as lower MW, higher cell permeability, and better oral absorption. Therefore, molecular glues offer an attractive approach to drug discovery in targeted protein degradation.

### SUMMARY OF THE DISCLOSURE

Provided herein is a compound of Formula (I):

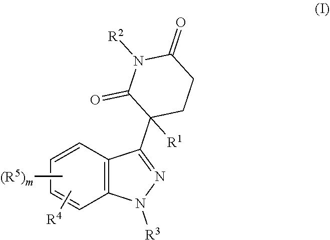

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ is hydrogen, deuterium, C₁₋₆ alkyl, or C₁₋₆ heteroalkyl;
R² is hydrogen or C₁₋₆ alkyl;
R³ and R⁴ are:
   (i) R³ is

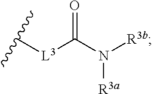

and
R⁴ is hydrogen; or
(ii) R³ is hydrogen, C₁₋₆ alkyl, or C₁₋₆ heteroalkyl; and
R⁴ is

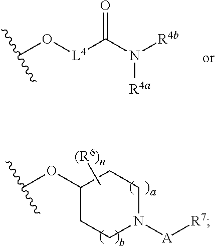

each R⁵ and R⁶ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1b}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR^{1d}, —NR^{1a}C(S)NR^{1b}R^{1c}, —NR^{1a} S(O)R^{1b}, —NR^{1a} S(O)₂R^{1b}, —NR^{1a} S(O)NR^{1b}R^{1c}, —NR^{1a} S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁷ is (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —OR^{1a}—OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1b}, —NR^{1a} S(O)₂R^{1b}, —NR^{1a} S(O)NR^{1b}R^{1c}, or —NR^{1a} S(O)₂NR^{1b}R^{1c};
R^{3a}, R^{3b}, R^{4a}, and R^{4b} are each independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c}; or R^{3a} and R^{3b} together with the N atom to which they are attached form heterocyclyl; or R^{4a} and R^{4b} together with the N atom to which they are attached form heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
A is —C(O)—, —C(O)C(O)—, —S(O)—, or —S(O₂)—;
L³ and L⁴ are each independently C₁₋₆ alkylene, C₁₋₆ heteroalkylene, C₃₋₁₀ cycloalkylene, or heterocyclylene;
a and b are each independently an integer of 0, 1, 2, or 3;
m is an integer of 0, 1, 2, or 3; and
n is an integer of 0, 1, 2, 3, 4, 5, 6, 7, or 8;
wherein each alkyl, alkylene, heteroalkyl, heteroalkylene, alkenyl, alkynyl, cycloalkyl, cycloalkylene, aryl, aralkyl, heteroaryl, heterocyclyl, and heterocyclylene is optionally substituted with one or more substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR)NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

Additionally, provided herein is a method of treating, preventing, or ameliorating one or more symptoms of a disorder, disease, or condition mediated by a zinc-figure protein, CK1α, GSPT1, and/or PDE6D in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore provided herein is a method of treating, preventing, or ameliorating one or more symptoms of a proliferative disease in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inhibiting the growth of a cell, comprising contacting the cell with an effective amount of a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inducing degradation of a protein, comprising contacting the protein with an effective amount of a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof; wherein the protein is a zinc-figure protein, CK1α, GSPT1, or PDE6D.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)+C(O)—, or —C(O)(CH₂)₅C(O)—.

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —CH₂CH₂O—, —CH₂CH₂CH₂O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃—, —(CH₂CH₂O)₄—, —(CH₂CH₂O)₅—, —CH₂NH—, —CH₂NHCH₂—, —CH₂CH₂NH—, CH₂CH₂CH₂NH—, —(CH₂)₄NH—, —CH₂S—, —CH₂SCH₂—, and —CH₂CH₂S—. Examples of substituted heteroalkylene groups include, but are not limited to, —C(O)CH₂O—, —C(O)(CH₂)₂O—, —C(O)CH₂CH₂CH₂O—, —C(O)CH₂CH₂CH₂CH₂O—, —C(O)(CH₂)₅O—, —C(O)(CH₂)₆O—, —C(O)(CH₂)₇O—, —C(O)(CH₂)₈O—, —C(O)(CH₂)₉O—, —C(O)(CH₂)₁₀O—, —C(O)CH₂OCH₂CH₂O—, —C(O)CH₂O(CH₂CH₂O)₂—, —C(O)CH₂O(CH₂CH₂O)₃—, —C(O)CH₂O(CH₂CH₂O)₄, —C(O)CH₂O(CH₂CH₂O)₅—, —CH₂NHC(O)CH₂—, —CH₂CH₂C(O)NH—, —CH₂N(CH₃), (CH₂)₂N(CH₃)—, —(CH₂)₃N(CH₃), or —(CH₂)₄N(CH₃)—.

The term “alkenyl” refers to a linear or branched monovalent hydrocarbon radical, which contains one or more in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z′ or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The term “alkynyl” refers to a linear or branched monovalent hydrocarbon radical, which contains one or more in another embodiment, one, carbon-carbon triple bond(s). An alkynyl group does not contain a carbon-carbon double bond. The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused and/or spiro bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkanediyl groups include, but are not limited to, cyclopropanediyl (including all isomeric forms, e.g., cyclopropane-1,1-diyl and cyclopropane-1,2-diyl), cyclobutanediyl (including all isomeric forms, e.g., cyclobutane-1,1-diyl, cyclobutane-1,2-diyl, and cyclobutane-1,3-diyl), cyclopentanediyl (including all isomeric forms, e.g., cyclopentane-1,1-diyl, cyclopentane-1,2-diyl, and cyclopentane-1,3-diyl), cyclohexanediyl (including all isomeric forms, e.g., cyclohexane-1,1-diyl, cyclohexane-1,2-diyl, cyclohexane-1,3-diyl, and cyclohex-1,4-diyl), cycloheptanediyl (including all isomeric forms, e.g., cycloheptane-1,1-diyl, cycloheptane-1,2-diyl, cycloheptane-1,3-diyl, and cycloheptane-1,4-diyl), decalinediyl (including all isomeric forms, e.g., decaline-1,1-diyl, decaline-1,2-diyl, and decaline-1,8-diyl), and adamantdiyl (including all isomeric forms, e.g., adamant-1,2-diyl, adamant-1,3-diyl, and adamant-1,8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. For a heterocyclylene group containing a heteroaromatic ring and a nonaromatic heterocyclic ring, the heterocyclylene group has at least one bond to the rest of a molecule via its nonaromatic heterocyclic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydro-pyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydro-indoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydro-thiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen”, “halide,” or “halo” refers to fluoro, chloro, bromo, and/or iodo.

The term “optionally substituted” is intended to mean that a group or substituent, such as an alkyl, alkylene, heteroalkyl, heteroalkylene, alkenyl, alkynyl, cycloalkyl, cycloalkylene, aryl, aralkyl, heteroaryl, heterocyclyl, or heterocyclylene group, may be substituted with one or more substituents Q, each of which is independently selected from, e.g., (a) deuterium (—D), cyano (—CN), halo, imino (═NH), nitro (—NO₂), and oxo (—O); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NRDR^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{e}, and R^{d} is independently (i) hydrogen or deuterium; (ii) C₁₋₆ alkyl, C₁₋₆ C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{c}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (1H), deuterium (2H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (1H), deuterium (2H), carbon-12 (¹²C), carbon-13 (¹³C), nitrogen-14 (14N), nitrogen-15 (¹⁵N), oxygen-16 (¹⁶O), oxygen-17 (17O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (14O), oxygen-15 (15O), fluorine-18 (¹⁸F), phosphorus-32 (³²P), phosphorus-33 (³³P), sulfur-35 (³⁵S), chlorine-36 (³⁶Cl), iodine-123 (123I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (1H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH—represents both —C(O)NH—and —NHC(O)—.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, provided herein is a compound of Formula (I):

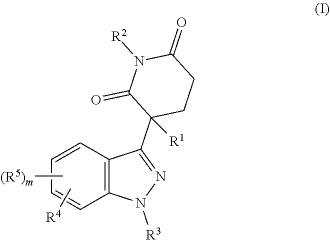

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ is hydrogen, deuterium, C₁₋₆ alkyl, or C₁₋₆ heteroalkyl;
R² is hydrogen or C₁₋₆ alkyl;
R³ and R⁴ are:
   (iii) R³ is

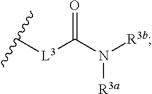

and
R⁴ is hydrogen; or
(iv) R³ is hydrogen, C₁₋₆ alkyl, or C₁₋₆ heteroalkyl; and
R⁴ is

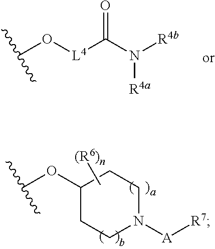

each R⁵ and R⁶ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR^{1d}, —NR^{1a}C(S)NR^{1b}R^{1c}, —NR^{1a} S(O)R^{1b}, —NR^{1a} S(O)₂R^{1b}, —NR^{1a} S(O)NR^{1b}R^{1c}, —NR^{1a} S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁷ is (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR^{1d}, —NR^{1a}C(S)NR^{1b}R^{1c}, —NR^{1a} S(O)R^{1b}, —NR^{1a} S(O)₂R^{1b}, —NR^{1a} S(O)NR^{1b}R^{1c}, or —NR^{1a} S(O)₂NR^{1b}R^{1c},
R^{3a}, R^{3b}, R^{4a}, and R^{4b} are each independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c}; or R^{3a} and R^{3b} together with the N atom to which they are attached form heterocyclyl; or R^{4a} and R^{4b} together with the N atom to which they are attached form heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
A is —C(O)—, —C(O)C(O)—, —S(O)—, or —S(O₂)—;
L³ and L⁴ are each independently C₁₋₆ alkylene, C₁₋₆ heteroalkylene, C₃₋₁₀ cycloalkylene, or heterocyclylene;
a and b are each independently an integer of 0, 1, 2, or 3;
m is an integer of 0, 1, 2, or 3; and
n is an integer of 0, 1, 2, 3, 4, 5, 6, 7, or 8;
wherein each alkyl, alkylene, heteroalkyl, heteroalkylene, alkenyl, alkynyl, cycloalkyl, cycloalkylene, aryl, aralkyl, heteroaryl, heterocyclyl, and heterocyclylene is optionally substituted with one or more substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR4)NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{e}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{c}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{h}, —NR^{e}C(NR^{h})NR^{f}R^{g}, —NR^{e}C(S)R^{h}, —NR^{e}C(S)OR^{h}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In another embodiment, provided herein is a compound of Formula (II):

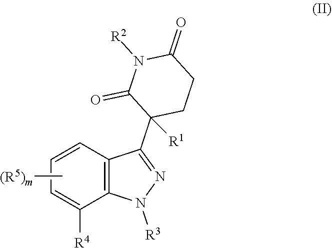

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³ is (i) hydrogen; or (ii) C₁₋₆ alkyl or C₁₋₆ heteroalkyl, each optionally substituted with one or more substituents Q; R⁴ is

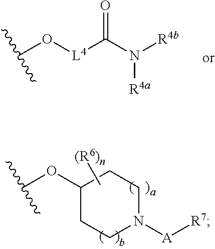

and R¹, R², R⁵, R⁶, R⁷, R^{4a}, R^{4b}, A, L⁴, a, b, m, and n are each as defined herein.

In yet another embodiment, provided herein is a compound of Formula (III):

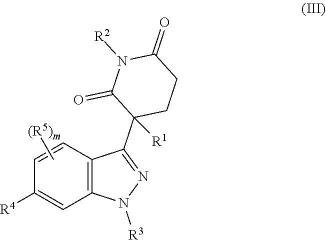

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³ is (i) hydrogen; or (ii) C₁₋₆ alkyl or C₁₋₆ heteroalkyl, each optionally substituted with one or more substituents Q; R⁴ is

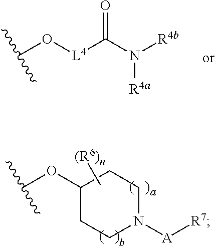

and R¹, R², R⁵, R⁶, R⁷, a, b, m, and n are each as defined herein.

In certain embodiments, in Formula (I), (II), or (III), R³ is (i) hydrogen; or (ii) C₁₋₆ alkyl or C₁₋₆ heteroalkyl, each optionally substituted with one or more substituents Q; and R⁴ is

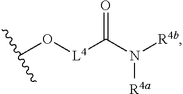

wherein R^{4a}, R^{4b}, and L⁴ are each as defined herein. In certain embodiments, in Formula (I), (II), or (III), R³ is (i) hydrogen; or (ii) C₁₋₆ alkyl or C₁₋₆ heteroalkyl, each optionally substituted with one or more substituents Q; and R⁴ is

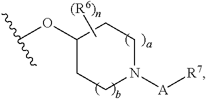

wherein R⁶, R⁷, a, b, and n are each as defined herein.

In yet another embodiment, provided herein is a compound of Formula (IV):

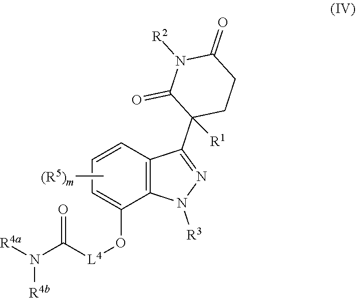

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{4a}, R^{4b}, L⁴, and m are each as defined herein.

In yet another embodiment, provided herein is a compound of Formula (V):

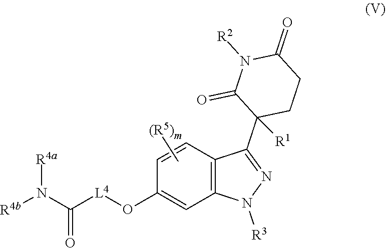

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{4a}, R^{4b}, L⁴, and m are each as defined herein.

In certain embodiments, in any one of Formulae (I) to (V), L⁴ is C₁-6 alkylene or C₁₋₆ heteroalkylene, each optionally substituted with one or more substituents Q. In certain embodiments, in any one of Formulae (I) to (V), L⁴ is C₁-6 alkylene, optionally substituted with one, two, or three substituents Q. In certain embodiments, in any one of Formulae (I) to (V), L⁴ is methanediyl, ethanediyl, or propanediyl, each optionally substituted with one, two, or three substituents Q. In certain embodiments, in any one of Formulae (I) to (V), L⁴ is methanediyl, optionally substituted with one, two, or three substituents Q.

In yet another embodiment, provided herein is a compound of Formula (VI):

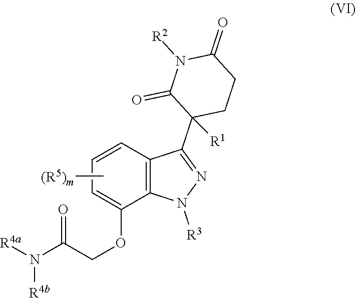

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{4a}, R^{4b}, and m are each as defined herein.

In yet another embodiment, provided herein is a compound of Formula (VII):

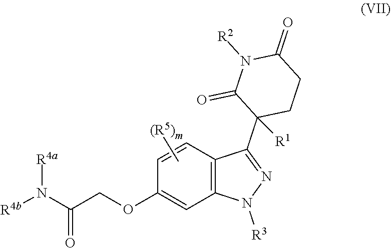

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{4a}, R^{4b}, and m are each as defined herein.

In certain embodiments, in any one of Formulae (I) to (VII), R^{4a} is hydrogen; or C₁₋₆ alkyl, optionally substituted with one or more substituents Q. In certain embodiments, in any one of Formulae (I) to (VII), R^{4a} is hydrogen. In certain embodiments, in any one of Formulae (I) to (VII), R^{4a} is C₁₋₆ alkyl, optionally substituted with one, two, or three substituents Q. In certain embodiments, in any one of Formulae (I) to (VII), R^{4a} is methyl, optionally substituted with one, two, or three substituents Q.

In certain embodiments, in any one of Formulae (I) to (VII), R^{4b} is C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one or more substituents Q. In certain embodiments, in any one of Formulae (I) to (VII), R^{4b} is C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (I) to (VII), R^{4b} is C₁₋₆ alkyl, optionally substituted with one, two, or three substituents Q. In certain embodiments, in any one of Formulae (I) to (VII), R^{4b} is methyl, ethyl, butyl, pentyl, or hexyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1d}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (VII), R^{4b} is methyl, ethyl, butyl, pentyl, or hexyl, each optionally substituted with one, two, or three substituents, each of which is independently cyano, cyclopropyl, 1-hydroxycyclopropyl, cyclopentyl, 1-cyanocyclopentyl, 3-hydroxycyclopentyl, 5-methylfuran-2-yl, 1-methylimidazol-5-yl, 5-methylisoxazol-3-yl, 1-methylpyrazol-3-yl, 1,5-dimethylpyrazol-3-yl, thien-2-yl, thiazol-2-yl, thiazol-4-yl, 2-chlorothiazol-5-yl, 5-isopropyl-1,2,4-oxadiazol-3-yl, 3-cyclohexyl-1,2,4-oxadiazol-5-yl, 5-phenyl-1,2,4-oxadiazol-3-yl, 3,5-dimethyl-1,2,4-triazol-1-yl, pyridin-2-yl, 6-ethoxypyridin-3-yl, 1-methyl-2-oxo-1,2-dihydropyridin-4-yl, 1-methylindol-3-yl, 2-methyl-indol-5-yl, oxetan-2-yl, 5-oxopyrrolidin-2-yl, 1-methylpyrrolidin-3-yl, tetrahydrofuran-2-yl, 4-hydroxytetrahydropyran-4-yl, 4-benzylmorpholin-2-yl, 1,1-dioxidothiomorpholino, methyl-aminocarbonyl, cyclopentyloxy, p-tolyloxy, benzyloxy, or sulfamoyl.

In certain embodiments, in any one of Formulae (I) to (VII), R^{4b} is neopentyl, 2-cyanoethyl, cyclopropylmethyl, (1-hydroxycyclopropyl)methyl, cyclopentylmethyl, (1-cyano-cyclopentyl)methyl, (3-hydroxycyclopentyl)methyl, 1-(5-methylfuran-2-yl) propyl, thien-2-yl-methyl, (1-methylimidazol-5-yl)methyl, (5-methylisoxazol-3-yl)methyl, (1-methylpyrazol-3-yl)-methyl, (1,5-dimethylpyrazol-3-yl)methyl, thiazol-2-ylmethyl, thiazol-4-ylmethyl, (2-chloro-thiazol-5-yl)methyl, (3-methyl-1,2,4-oxadiazol-5-yl)methyl, 2-(5-isopropyl-1,2,4-oxadiazol-3-yl)ethyl, (3-cyclohexyl-1,2,4-oxadiazol-5-yl)methyl, (5-phenyl-1,2,4-oxadiazol-3-yl)methyl, 2-(3,5-dimethyl-1,2,4-triazol-1-yl)ethyl, 1-(pyridin-2-yl)ethyl, (6-ethoxypyridin-3-yl)methyl, (1-methyl-2-oxo-1,2-dihydropyridin-4-yl)methyl, (1-methylindol-3-yl)methyl, (2-methylindol-5-yl)methyl, (oxetan-2-yl)methyl, (5-oxopyrrolidin-2-yl)methyl, (1-methylpyrrolidin-3-yl)methyl, (tetrahydrofuran-2-yl)methyl, (4-hydroxytetrahydropyran-4-yl)methyl, (4-benzylmorpholin-2-yl)methyl, 3-(1,1-dioxidothiomorpholino) propyl, methylaminocarbonylmethyl, 2-(cyclopentyl-oxy)ethyl, 2-(p-tolyloxy)ethyl, 2-(benzyloxy)ethyl, or 2-sulfamoylethyl.

In certain embodiments, in any one of Formulae (I) to (VII), R^{4b} is C₃₋₁₀ cycloalkyl, optionally substituted with one, two, or three substituents Q. In certain embodiments, in any one of Formulae (I) to (VII), R^{4b} is monocyclic C₃₋₁₀ cycloalkyl, optionally substituted with one, two, or three substituents Q. In certain embodiments, in any one of Formulae (I) to (VII), R^{4b} is bicyclic C₄₋₁₀ cycloalkyl, optionally substituted with one, two, or three substituents Q. In certain embodiments, in any one of Formulae (I) to (VII), R^{4b} is bridged, fused, or spiro C₄₋₁₀ cycloalkyl, each optionally substituted with one, two, or three substituents Q. In certain embodiments, in any one of Formulae (I) to (VII), R^{4b} is cyclopropyl, cyclobutyl, cyclopentyl, cyclohexyl, bicyclo[1.1.1]pentanyl, or bicyclo[2.2.2]octanyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{a}, R^{1b}, R^{1c}, and R^{1d} is as defined herein. In certain embodiments, in any one of Formulae (I) to (VII), R^{4b} is cyclopropyl, cyclobutyl, cyclopentyl, cyclohexyl, bicyclo[1.1.1]pentan-1-yl, or bicyclo[2.2.2]octan-1-yl, each optionally substituted with one, two, or three substituents, each of which is independently cyano, fluoro, hydroxyl, or methoxy. In certain embodiments, in any one of Formulae (I) to (VII), R^{4b} is cyclopropyl, cyclobutyl, cyclopentyl, 3-hydroxycyclopentyl, cyclohexyl, 4-hydroxycyclohexyl, 4-methoxycyclohexyl, 3-fluorobicyclo[1.1.1]pentan-1-yl, or 4-cyanobicyclo[2.2.2]octan-1-yl.

In certain embodiments, in any one of Formulae (I) to (VII), R^{4b} is C₆₋₁₄ aryl, optionally substituted with one, two, or three substituents Q. In certain embodiments, in any one of Formulae (I) to (VII), R^{4b} is phenyl, optionally substituted with one, two, or three substituents Q. In certain embodiments, in any one of Formulae (I) to (VII), R^{4b} is bicyclic C₈₋₁₄ aryl, optionally substituted with one, two, or three substituents Q. In certain embodiments, in any one of Formulae (I) to (VII), R^{4b} is phenyl, 2,3-dihydroindenyl, or naphthalenyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (VII), R^{4b} is phenyl, 2,3-dihydroinden-1-yl, or naphthalen-1-yl, each optionally substituted with one, two, or three substituents, wherein each substituent is independently cyano, fluoro, chloro, bromo, methyl, trifluoromethyl, tert-butyl, (pyrrolidin-1-ylsulfonyl)methyl, pyrrol-1-yl, imidazol-1-yl, imidazol-2-yl, imidazol-5-yl, 2-methylimidazol-1-yl, oxazol-5-yl, 1-methylpiperidin-4-yl, 4-methylpiperazin-1-yl, morpholino, carboxy, hydroxyl, methoxy, isopropoxy, trifluoromethoxy, p-tolyloxy, methylsulfonyl, or methylsulfamoyl.

In certain embodiments, in any one of Formulae (I) to (VII), R^{4b} is 3-(ter-butyl)-phenyl, 4-((pyrrolidin-1-ylsulfonyl)methyl)phenyl, 4-(pyrrol-1-yl)phenyl, 3-(imidazol-1-yl)-phenyl, 4-(imidazol-2-yl)phenyl, 4-(imidazol-5-yl)phenyl, 4-(2-methylimidazol-1-yl)phenyl, 3-(oxazol-5-yl)phenyl, 4-(oxazol-5-yl)phenyl, 4-(1-methylpiperidin-4-yl)phenyl, 4-(4-methyl-piperazin-1-yl)phenyl, 3-isopropoxyphenyl, 4-(trifluoromethoxy)phenyl, 4-(p-tolyloxy)phenyl, 4-(methylsulfonyl)phenyl, 4-(methylsulfamoyl)phenyl, 2-cyano-3-fluorophenyl, 2-cyano-6-fluorophenyl, 2-cyano-4-fluorophenyl, 5-chloro-2-cyanophenyl, 3-bromo-2-cyanophenyl, 2-cyano-3-methylphenyl, 2-cyano-4-methylphenyl, 2-cyano-5-methylphenyl, 2-cyano-6-methyl-phenyl, 2-cyano-5-(trifluoromethyl)phenyl, 3-cyano-5-(trifluoromethyl)phenyl, 2-cyano-3-methoxyphenyl, 2-cyano-4-methoxyphenyl, 3-fluoro-5-morpholinophenyl, 2-cyano-4-carboxy-phenyl, 4-cyano-2-carboxyphenyl, 2-cyano-4,6-difluorophenyl, or 2-hydroxy-2,3-dihydroinden-1-yl, or 2-cyanonaphthalen-1-yl.

In certain embodiments, in any one of Formulae (I) to (VII), R^{4b} is C₇₋₁₅ aralkyl, optionally substituted with one, two, or three substituents Q. In certain embodiments, in any one of Formulae (I) to (VII), R^{4b} is monocyclic C₇₋₁₅ aralkyl, optionally substituted with one, two, or three substituents Q. In certain embodiments, in any one of Formulae (I) to (VII), R^{4b} is bicyclic C₉₋₁₅ aralkyl, optionally substituted with one, two, or three substituents Q. In certain embodiments, in any one of Formulae (I) to (VII), R^{4b} is benzyl, phenylethyl, or phenylpropyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, OR^{1a}, —NR^{1b}R^{1c}, —NR^{1a}C(O)R^{1d}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (VII), R^{4b} is benzyl, phenylethyl, or phenylpropyl, each optionally substituted with one, two, or three substituents, each of which is independently cyano, fluoro, chloro, methyl, isopropyl, hydroxyl, methoxy, butoxy, or sulfamoyl. In certain embodiments, in any one of Formulae (I) to (VII), R^{4b} is 2-cyanobenzyl, 3-cyanobenzyl, 4-isopropylbenzyl, 4-(morpholinomethyl)benzyl, 1-(4-fluorophenyl)ethyl, 1-(4-chlorophenyl)ethyl, 1-(p-tolyl)ethyl, 1-phenylpropyl, 4-(2-oxopyrrolidin-1-yl)benzyl, 4-hydroxybenzyl, 4-hydroxy-phenethyl, 4-(sec-butoxy)benzyl, 2,4-difluorobenzyl, 2,5-difluorobenzyl, 3,5-dichlorobenzyl, 3,5-dimethoxybenzyl, or 4-sulfamoylphenethyl.

In certain embodiments, in any one of Formulae (I) to (VII), R^{4b} is heteroaryl, optionally substituted with one, two, or three substituents Q. In certain embodiments, in any one of Formulae (I) to (VII), R^{4b} is monocyclic heteroaryl, optionally substituted with one, two, or three substituents Q. In certain embodiments, in any one of Formulae (I) to (VII), R^{4b} is 3-, 4-, 5-, 6-, or 7-membered heteroaryl, each optionally substituted with one, two, or three substituents Q. In certain embodiments, in any one of Formulae (I) to (VII), R^{4b} is 3-membered heteroaryl, optionally substituted with one, two, or three substituents Q. In certain embodiments, in any one of Formulae (I) to (VII), R^{4b} is 4-membered heteroaryl, optionally substituted with one, two, or three substituents Q. In certain embodiments, in any one of Formulae (I) to (VII), R^{4b} is 5-membered heteroaryl, optionally substituted with one, two, or three substituents Q. In certain embodiments, in any one of Formulae (I) to (VII), R^{4b} is 6-membered heteroaryl, optionally substituted with one, two, or three substituents Q. In certain embodiments, in any one of Formulae (I) to (VII), R^{4b} is 7-membered heteroaryl, optionally substituted with one, two, or three substituents Q. In certain embodiments, in any one of Formulae (I) to (VII), R^{4b} is bicyclic heteroaryl, optionally substituted with one, two, or three substituents Q. In certain embodiments, in any one of Formulae (I) to (VII), R^{4b} is 5,5-, 5,6-, or 6,6-fused heteroaryl, each optionally substituted with one, two, or three substituents Q. In certain embodiments, in any one of Formulae (I) to (VII), R^{4b} is 5,5-fused heteroaryl, optionally substituted with one, two, or three substituents Q. In certain embodiments, in any one of Formulae (I) to (VII), R^{4b} is 5,6-fused heteroaryl, optionally substituted with one, two, or three substituents Q. In certain embodiments, in any one of Formulae (I) to (VII), R^{4b} is 6,6-fused heteroaryl, optionally substituted with one, two, or three substituents Q.

In certain embodiments, in any one of Formulae (I) to (VII), R^{4b} is pyrrolyl, thienyl, isoxazolyl, isothiazolyl, oxazolyl, pyrrolyl, pyrazolyl, thiazolyl, 1,2,4-triazolyl, 1,2,5-oxadiazolyl, 1,3,4-oxadiazolyl, 1,3,4-thiadiazolyl, pyridinyl, pyridazinyl, pyrimidinyl, thieno-[3,2-c]pyrazolyl, indolyl, 4,5,6,7-tetrahydrobenzo[b]thienyl, 4,7-dihydrothieno[2,3-c]pyridinyl, or [1,2,4]triazolo[4,3-a]pyridinyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a}S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (VII), R^{4b} is pyrrol-2-yl, pyrrol-3-yl, thien-2-yl, thien-3-yl, thien-4-yl, thien-5-yl, isoxazol-3-yl, isoxazol-4-yl, isoxazol-5-yl, isothiazol-4-yl, isothiazol-5-yl, oxazol-2-yl, pyrrol-2-yl, pyrazol-4-yl, pyrazol-5-yl, thiazol-2-yl, thiazol-4-yl, thiazol-5-yl, 1,2,4-triazol-5-yl, 1,2,5-oxadiazol-3-yl, 1,3,4-oxadiazol-2-yl, 1,3,4-thiadiazol-2-yl, pyridin-2-yl, pyridin-3-yl, pyridazin-3-yl, pyrimidin-5-yl, thieno[3,2-c]pyrazol-3-yl, indol-2-yl, 4,5,6,7-tetrahydrobenzo[b]thien-2-yl, 4,7-dihydrothieno[2,3-c]pyridin-2-yl, or [1,2,4]triazolo-[4,3-a]pyridin-3-yl, each optionally substituted with one, two, or three substituents, each of which is independently cyano, chloro, iodo, methyl, ethyl, isopropyl, ter-butyl, trifluoromethyl, 2-hydroxyethyl, cyclopropyl, phenyl, 3-chlorophenyl, furan-2-yl, pyrazol-1-yl, pyridin-2-yl, oxetan-3-yl, tetrahydropyran-4-yl, 2-chlorobenzoyl, methoxycarbonyl, ethoxycarbonyl, aminocarbonyl, isopropylaminocarbonyl, difluoromethoxy, amino, or methylthio.

In certain embodiments, in any one of Formulae (I) to (VII), R^{4b} is 3-cyanopyrrol-2-yl, 3-cyano-4-methylpyrrol-2-yl, 2,4-diethoxycarbonylpyrrol-3-yl, 3-cyano-4,5-dimethyl-pyrrol-2-yl, 3-cyanothien-2-yl, 2-cyanothien-3-yl, 3-cyano-4-methylthien-2-yl, 3-(2-chloro-benzoyl)-5-ethylthien-2-yl, 2-methyl-3-methoxycarbonylthien-4-yl, 4-isopropyl-3-ethoxy-carbonylthien-2-yl, 3-ethoxycarbonyl-4-phenylthien-2-yl, 3,4-diethoxycarbonylthien-2-yl, 5-cyano-3-ethoxycarbonyl-2-(methylthio) thien-4-yl, 3-methyl-2-methoxycarbonylthien-5-yl, 3-cyano-4,5-dimethylthien-2-yl, 3-methoxycarbonyl-4,5-dimethylthien-2-yl, 5-iodo-2-methoxy-carbonylthien-3-yl, isoxazol-5-yl, 3-methylisoxazol-5-yl, 3-ethylisoxazol-5-yl, 5-ethylisoxazol-3-yl, 4-cyano-3-methylisoxazol-5-yl, 3,5-dimethylisoxazol-4-yl, 3-methylisothiazol-4-yl, 3-methylisothiazol-5-yl, 4-cyano-3-methylisothiazol-5-yl, 3-(isopropylaminocarbonyl)-4-amino-isothiazol-5-yl, 3-aminocarbonyl-4-aminoisothiazol-5-yl, oxazol-2-yl, 1-methylpyrazol-4-yl, 1-methylpyrazol-5-yl, 3-methylpyrazol-5-yl, 1-isopropylpyrazol-4-yl, 3-cyclopropylpyrazol-5-yl, 1-phenylpyrazol-4-yl, 3-phenylpyrazol-4-yl, 4-(3-chlorophenyl) pyrazol-5-yl, 3-(pyridin-2-yl)-pyrazol-4-yl, 1-(oxetan-3-yl) pyrazol-4-yl, 1-(tetrahydropyran-4-yl) pyrazol-4-yl, 3-(difluoro-methoxy) pyrazol-5-yl, 4-cyano-1-methylpyrazol-5-yl, 4-cyano-1-(2-hydroxyethyl) pyrazol-5-yl, 4-cyano-1-phenylpyrazol-5-yl, 3,5-dimethylpyrazol-4-yl, 1-(tert-butyl)-3-methylpyrazol-5-yl, 1-methyl-3-(trifluoromethyl) pyrazol-4-yl, 1-methyl-3-(trifluoromethyl) pyrazol-5-yl, 3-cyclo-propyl-1-methylpyrazol-5-yl, 3-(tert-butyl)-1-phenylpyrazol-5-yl, 4-ethoxycarbonyl-3-methyl-pyrazol-5-yl, 4-amino-1-methylpyrazol-5-yl, 4-cyano-1,3-dimethylpyrazol-5-yl, 4-cyano-3-methyl-1-phenylpyrazol-5-yl, thiazol-4-yl, 4-(trifluoromethyl) thiazol-2-yl, 4-(tert-butyl) thiazol-2-yl, 2-methylthiazol-5-yl, 4-ethoxycarbonylthiazol-5-yl, 1-methyl-1,2,4-triazol-5-yl, 4-methyl-1,2,5-oxadiazol-3-yl, 5-methyl-1,3,4-oxadiazol-2-yl, 5-(furan-2-yl)-1,3,4-oxadiazol-2-yl, 5-(tert-butyl)-1,3,4-thiadiazol-2-yl, pyridin-2-yl, 2-cyanopyridin-3-yl, 4-(pyrazol-1-yl)pyridin-2-yl, 6-chloro-3-cyanopyridin-2-yl, 2-cyano-5-methylpyridin-3-yl, pyridazin-3-yl, pyrimidin-5-yl, 3-methoxycarbonylindol-2-yl, 1-methyl-5-methoxycarbonylthieno[3,2-c]pyrazol-3-yl, 3-cyano-6-methyl-4,5,6,7-tetrahydrobenzo[b]thien-2-yl, 3-methoxycarbonyl-4,5,6,7-tetrahydrobenzo[b]-thien-2-yl, 3-ethoxycarbonyl-4-methyl-4,5,6,7-tetrahydrobenzo[b]thien-2-yl, 6-tert-butoxy-carboyl-3-ethoxycarbonyl-4,7-dihydrothieno[2,3-c]pyridin-2-yl, or 8-methyl[1,2,4]triazolo[4,3-a]pyridin-3-yl.

In certain embodiments, in any one of Formulae (I) to (VII), R^{4b} is heterocyclyl, optionally substituted with one, two, or three substituents Q. In certain embodiments, in any one of Formulae (I) to (VII), R^{4b} is monocyclic heterocyclyl, optionally substituted with one, two, or three substituents Q. In certain embodiments, in any one of Formulae (I) to (VII), R^{4b} is 3-, 4-, 5-, 6-, or 7-membered heterocyclyl, each optionally substituted with one, two, or three substituents Q. In certain embodiments, in any one of Formulae (I) to (VII), R^{4b} is 3-membered heterocyclyl, optionally substituted with one, two, or three substituents Q. In certain embodiments, in any one of Formulae (I) to (VII), R^{4b} is 4-membered heterocyclyl, optionally substituted with one, two, or three substituents Q. In certain embodiments, in any one of Formulae (I) to (VII), R^{4b} is 5-membered heterocyclyl, optionally substituted with one, two, or three substituents Q. In certain embodiments, in any one of Formulae (I) to (VII), R^{4b} is 6-membered heterocyclyl, optionally substituted with one, two, or three substituents Q. In certain embodiments, in any one of Formulae (I) to (VII), R^{4b} is 7-membered heterocyclyl, optionally substituted with one, two, or three substituents Q. In certain embodiments, in any one of Formulae (I) to (VII), R^{4b} is bicyclic heterocyclyl, optionally substituted with one, two, or three substituents Q. In certain embodiments, in any one of Formulae (I) to (VII), R^{4b} is bridged, fused, or spiro heterocyclyl, each optionally substituted with one, two, or three substituents Q. In certain embodiments, in any one of Formulae (I) to (VII), R^{4b} is bridged heterocyclyl, optionally substituted with one, two, or three substituents Q. In certain embodiments, in any one of Formulae (I) to (VII), R^{4b} is fused heterocyclyl, optionally substituted with one, two, or three substituents Q. In certain embodiments, in any one of Formulae (I) to (VII), R^{4b} is spiro heterocyclyl, optionally substituted with one, two, or three substituents Q.

In certain embodiments, in any one of Formulae (I) to (VII), R^{4b} is oxetanyl, azetidinyl, pyrrolidinyl, tetrahydrofuranyl, tetrahydropyranyl, piperidinyl, piperidinyl, thietanyl, tetrahydrothiopyranyl, or azepanyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a}S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (VII), R^{4b} is oxetan-3-yl, azetidin-3-yl, pyrrolidin-3-yl, tetrahydrofuran-3-yl, tetrahydropyran-4-yl, piperidin-3-yl, piperidin-4-yl, thietan-3-yl, tetrahydrothiopyran-4-yl, or azepan-3-yl, each optionally substituted with one, two, or three substituents, each of which is independently oxo, methyl, propyl, or benzyl. In certain embodiments, in any one of Formulae (I) to (VII), R^{4b} is oxetan-3-yl, 1-methylazetidin-3-yl, 1-methylpyrrolidin-3-yl, 1-benzylpyrrolidin-3-yl, 2-oxopyrrolidin-3-yl, tetrahydrofuran-3-yl, 2,3-dimethyltetrahydrofuran-3-yl, tetrahydropyran-4-yl, 4-methyltetrahydropyran-4-yl, 1-methyl-piperidin-4-yl, 1-propylpiperidin-4-yl, 2-oxopiperidin-3-yl, 1-methyl-2-oxopiperidin-4-yl, 1,1-dioxidothietan-3-yl, 1,1-dioxidotetrahydrothiopyran-4-yl, or 2-oxoazepan-3-yl.

In certain embodiments, in any one of Formulae (I) to (VII), R^{4a} and R^{4b} together with the N atom to which they are attached form heterocyclyl, optionally substituted with one, two, or three substituents Q. In certain embodiments, in any one of Formulae (I) to (VII), R^{4a} and R^{4b} together with the N atom to which they are attached form monocyclic heterocyclyl, optionally substituted with one, two, or three substituents Q. In certain embodiments, in any one of Formulae (I) to (VII), R^{4a} and R^{4b} together with the N atom to which they are attached form 3-, 4-, 5-, 6-, or 7-membered heterocyclyl, each optionally substituted with one, two, or three substituents Q. In certain embodiments, in any one of Formulae (I) to (VII), R^{4a} and R^{4b} together with the N atom to which they are attached form 3-membered heterocyclyl, optionally substituted with one, two, or three substituents Q. In certain embodiments, in any one of Formulae (I) to (VII), R^{4a} and R^{4b} together with the N atom to which they are attached form 4-membered heterocyclyl, optionally substituted with one, two, or three substituents Q. In certain embodiments, in any one of Formulae (I) to (VII), R^{4a} and R^{4b} together with the N atom to which they are attached form 5-membered heterocyclyl, optionally substituted with one, two, or three substituents Q. In certain embodiments, in any one of Formulae (I) to (VII), R^{4a} and R^{4b} together with the N atom to which they are attached form 6-membered heterocyclyl, optionally substituted with one, two, or three substituents Q. In certain embodiments, in any one of Formulae (I) to (VII), R^{4a} and R^{4b} together with the N atom to which they are attached form 7-membered heterocyclyl, optionally substituted with one, two, or three substituents Q. In certain embodiments, in any one of Formulae (I) to (VII), R^{4a} and R^{4b} together with the N atom to which they are attached form piperazinyl, optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a}S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (I) to (VII), R^{4a} and R^{4b} together with the N atom to which they are attached form bicyclic heterocyclyl, optionally substituted with one, two, or three substituents Q. In certain embodiments, in any one of Formulae (I) to (VII), R^{4a} and R^{4b} together with the N atom to which they are attached form bridged, fused, or spiro heterocyclyl, each optionally substituted with one, two, or three substituents Q. In certain embodiments, in any one of Formulae (I) to (VII), R^{4a} and R^{4b} together with the N atom to which they are attached form bridged heterocyclyl, optionally substituted with one, two, or three substituents Q. In certain embodiments, in any one of Formulae (I) to (VII), R^{4a} and R^{4b} together with the N atom to which they are attached form fused heterocyclyl, optionally substituted with one, two, or three substituents Q. In certain embodiments, in any one of Formulae (I) to (VII), R^{4a} and R^{4b} together with the N atom to which they are attached form spiro heterocyclyl, optionally substituted with one, two, or three substituents Q.

In yet another embodiment, provided herein is a compound of Formula (VIII):

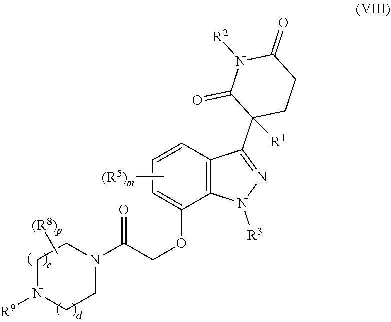

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each R^{g} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR^{1d}, —NR^{1a}C(S)NR^{1b}R^{1c}, —NR^{1a} S(O)R^{1d}, —NR^{1a} S(O)₂R^{1d}, —NR^{1a} S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c},
R⁹ is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
c and d are each independently an integer of 0, 1, 2, or 3;
p is an integer of 0, 1, 2, 3, 4, 5, 6, 7, or 8; and
R¹, R², R³, R⁵, and m are each as defined herein.

In yet another embodiment, provided herein is a compound of Formula (IX):

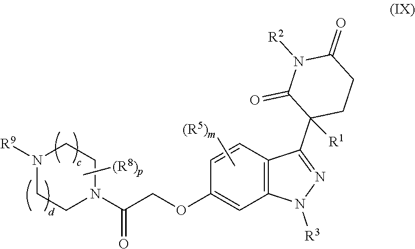

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{g}, R⁹, c, d, m, and p are each as defined herein.

In certain embodiments, in Formula (VIII) or (IX), c and d are each independently an integer of 0, 1, or 2. In certain embodiments, in Formula (VIII) or (IX), c and d are each independently an integer of 0 or 1. In certain embodiments, in Formula (VIII) or (IX), c and d are each an integer of 1.

In yet another embodiment, provided herein is a compound of Formula (X):

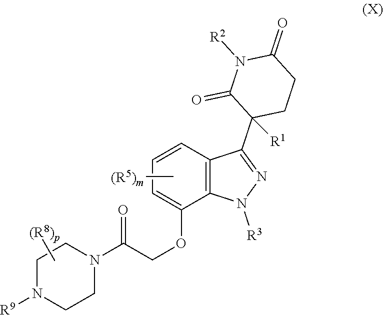

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{g}, R⁹, m, and p are each as defined herein.

In yet another embodiment, provided herein is a compound of Formula (XI):

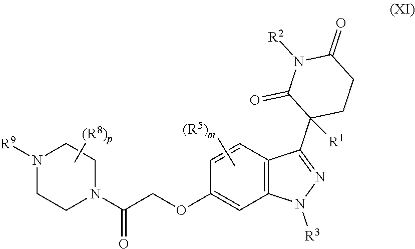

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R^{g}, R⁹, m, and p are each as defined herein.

In certain embodiments, in any one of Formulae (VIII) to (XI), R⁹ is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more substituents Q. In certain embodiments, in any one of Formulae (VIII) to (XI), R⁹ is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (VIII) to (XI), each R^{g} is independently halo. In certain embodiments, in any one of Formulae (VIII) to (XI), each R^{g} is independently fluoro or chloro. In certain embodiments, in any one of Formulae (VIII) to (XI), each R^{g} is fluoro.

In certain embodiments, in any one of Formulae (VIII) to (XI), R⁹ is C₁₋₆ alkyl, optionally substituted with one, two, or three substituents Q. In certain embodiments, in any one of Formulae (VIII) to (XI), R⁹ is methyl, ethyl, propyl, or butyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is methyl, ethyl, propyl, or butyl, each optionally substituted with one, two, or three substituents, each of which is independently fluoro, cyclopropyl, cyclobutyl, 4-fluorophenyl, 5-cyano-1-methylpyrrol-2-yl, pyrazol-1-yl, thien-2-yl, 2-mercapto-4-methylthiazol-5-yl, 4-oxo-2-thioxothiazolidin-3-yl, 1-methylpiperidin-4-yl, 1,1-dioxidothiomorpholino, piperazin-1-yl, hydroxyl, 2-cyanophenoxy, benzoxy, 2-benzoxy, methylamino, phenylacetamido, guanidino, phenylthio, 4-chlorophenylthio, p-tolylthio, 2,4-difluorophenylthio, or phenylsulfonyl.

In certain embodiments, in any one of Formulae (VIII) to (XI), R⁹ is cyclopropyl-methyl, cyclobutylmethyl, (4-fluorophenyl)difluoromethyl, (5-cyano-1-methylpyrrol-2-yl)-methyl, pyrazol-1-ylmethyl, thien-2-ylmethyl, (2-mercapto-4-methylthiazol-5-yl)methyl, (4-oxo-2-thioxothiazolidin-3-yl)methyl, (1-methylpiperidin-4-yl)methyl, (1,1-dioxidothiomorpholino)-methyl, piperazin-1-ylmethyl, 2-hydroxyethyl, (2-cyanophenoxy)methyl, benzoxymethyl, 2-benzoxyprop-2-yl, methylaminomethyl, phenylacetamidomethyl, guanidinomethyl, 2-guanidino-ethyl, phenylthiomethyl, (4-chlorophenylthio)methyl, (p-tolylthio)methyl, (2,4-difluorophenyl-thio)methyl, or 2-(phenylsulfonyl)ethyl.

In certain embodiments, in any one of Formulae (VIII) to (XI), R⁹ is C₂₋₆ alkenyl, optionally substituted with one, two, or three substituents Q. In certain embodiments, in any one of Formulae (VIII) to (XI), R⁹ is ethenyl, propenyl, or butenyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is ethenyl, propenyl, or butenyl, each optionally substituted with one, two, or three substituents, each of which is independently phenyl, 4-trifluoromethylphenyl, 3,4-difluorophenyl, 4-aminophenyl, 5-methylthien-2-yl, thiazol-2-yl, benzofuran-2-yl, or benzo[d][1,3]-dioxol-5-yl. In certain embodiments, in any one of Formulae (VIII) to (XI), R⁹ is 1-butenyl, 2-phenylethenyl, 2-(4-trifluoromethylphenyl)ethenyl, 2-(3,4-difluorophenyl)ethenyl, 2-(4-aminophenyl)ethenyl, 2-(5-methylthien-2-yl)ethenyl, 2-(thiazol-2-yl)ethenyl, 2-(benzofuran-2-yl)ethenyl, or 2-(benzo[d]-[1,3]dioxol-5-yl)ethenyl.

In certain embodiments, in any one of Formulae (VIII) to (XI), R⁹ is C₂₋₆ alkynyl, optionally substituted with one, two, or three substituents Q. In certain embodiments, in any one of Formulae (VIII) to (XI), R⁹ is ethynyl, propynyl, or butynyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is 1-butynyl, phenylethynyl, (3-fluorophenyl)ethynyl, or (4-trifluoromethylphenyl)ethynyl.

In certain embodiments, in any one of Formulae (VIII) to (XI), R⁹ is C₃₋₁₀ cycloalkyl, optionally substituted with one, two, or three substituents Q. In certain embodiments, in any one of Formulae (VIII) to (XI), R⁹ is monocyclic C₃₋₁₀ cycloalkyl, optionally substituted with one, two, or three substituents Q. In certain embodiments, in any one of Formulae (VIII) to (XI), R⁹ is cyclopropyl, cyclobutyl, cyclopentyl, cyclohexyl, or cycloheptyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is bicyclic C₄₋₁₀ cycloalkyl, optionally substituted with one, two, or three substituents Q. In certain embodiments, in any one of Formulae (VIII) to (XI), R⁹ is bridged, fused, or spiro C₄₋₁₀ cycloalkyl, each optionally substituted with one, two, or three substituents Q. In certain embodiments, in any one of Formulae (VIII) to (XI), R⁹ is bridged C₄-10 cycloalkyl, optionally substituted with one, two, or three substituents Q. In certain embodiments, in any one of Formulae (VIII) to (XI), R⁹ is fused C₄₋₁₀ cycloalkyl, optionally substituted with one, two, or three substituents Q. In certain embodiments, in any one of Formulae (VIII) to (XI), R⁹ is spiro C₄₋₁₀ cycloalkyl, optionally substituted with one, two, or three substituents Q.

In certain embodiments, in any one of Formulae (VIII) to (XI), R⁹ is cyclopropyl, cyclobutyl, cyclobut-1-ene-1-yl, cyclopentyl, cyclohexyl, or bicyclo[1.1.1]pentyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is cyclopropyl, cyclobutyl, cyclobut-1-ene-1-yl, cyclopentyl, cyclohexyl, or bicyclo[1.1.1]pent-1-yl, each optionally substituted with one, two, or three substituents, each of which is independently cyano, hydroxymethyl, tert-butyl, phenyl, or hydroxyl. In certain embodiments, in any one of Formulae (VIII) to (XI), R⁹ is 1-(hydroxymethyl)cyclopropyl, 1-hydroxycyclopropyl, cyclobutyl, 1-cyanocyclobutyl, 3-phenyl-cyclobutyl, cyclobut-1-ene-1-yl, cyclopentyl, cyclohexyl, 4-hydroxycyclohexyl, bicyclo[1.1.1]-pent-1-yl, 3-cyanobicyclo[1.1.1]pent-1-yl, or 3-(tert-butyl) bicyclo[1.1.1]pent-1-yl.

In certain embodiments, in any one of Formulae (VIII) to (XI), R⁹ is C₆₋₁₄ aryl, optionally substituted with one, two, or three substituents Q. In certain embodiments, in any one of Formulae (VIII) to (XI), R⁹ is phenyl, optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a}S(O)₂R^{1d} —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is bicyclic C₈₋₁₄ aryl, optionally substituted with one, two, or three substituents Q. In certain embodiments, in any one of Formulae (VIII) to (XI), R⁹ is phenyl, optionally substituted with one, two, or three substituents, each of which is independently cyano, fluoro, nitro, phenylthiomethyl, methyl, tert-butyl, 4-fluorophenyl, imidazol-2-yl, pyrazol-1-yl, 5-methyl-1,2,4-oxadiazol-3-yl, hydroxyl, methoxy, trifluoromethoxy, isopropoxy, phenoxy, aminocarbonyl, amino, or phenylsulfonamido.

In certain embodiments, in any one of Formulae (VIII) to (XI), R⁹ is 4-cyano-phenyl, 4-fluorophenyl, 4-nitrophenyl, 2-(phenylthiomethyl)phenyl, 4-(tert-butyl)phenyl, 3-(4-fluorophenyl)phenyl, 4-(imidazol-2-yl)phenyl, 4-(pyrazol-1-yl)phenyl, 3-(5-methyl-1,2,4-oxadiazol-3-yl)phenyl, 2-methoxyphenyl, 3-methoxyphenyl, 4-methoxyphenyl, 2-trifluoro-methoxyphenyl, 3-trifluoromethoxyphenyl, 4-trifluoromethoxyphenyl, 3-isopropoxyphenyl, 4-isopropoxyphenyl, 3-phenoxyphenyl, 4-phenoxyphenyl, 4-aminocarbonylphenyl, 4-phenyl-sulfonamidophenyl, 4-cyano-2-fluorophenyl, 3-cyano-5-methylphenyl, 3-hydroxy-4-methoxy-phenyl, 4-hydroxy-3-trifluoromethoxyphenyl, or 3-amino-5-cyanophenyl.

In certain embodiments, in any one of Formulae (VIII) to (XI), R⁹ is heteroaryl, optionally substituted with one, two, or three substituents Q. In certain embodiments, in any one of Formulae (VIII) to (XI), R⁹ is monocyclic heteroaryl, optionally substituted with one, two, or three substituents Q. In certain embodiments, in any one of Formulae (VIII) to (XI), R⁹ is 5- or 6-membered heteroaryl, each optionally substituted with one, two, or three substituents Q. In certain embodiments, in any one of Formulae (VIII) to (XI), R⁹ is 5-membered heteroaryl, optionally substituted with one, two, or three substituents Q. In certain embodiments, in any one of Formulae (VIII) to (XI), R⁹ is 6-membered heteroaryl, optionally substituted with one, two, or three substituents Q. In certain embodiments, in any one of Formulae (VIII) to (XI), R⁹ is bicyclic heteroaryl, optionally substituted with one, two, or three substituents Q. In certain embodiments, in any one of Formulae (VIII) to (XI), R⁹ is 5,5-, 5,6, or 6,6-fused heteroaryl, each optionally substituted with one, two, or three substituents Q. In certain embodiments, in any one of Formulae (VIII) to (XI), R⁹ is 5,5-fused heteroaryl, optionally substituted with one, two, or three substituents Q. In certain embodiments, in any one of Formulae (VIII) to (XI), R⁹ is 5,6-fused heteroaryl, optionally substituted with one, two, or three substituents Q. In certain embodiments, in any one of Formulae (VIII) to (XI), R⁹ is 6,6-fused heteroaryl, optionally substituted with one, two, or three substituents Q.

In certain embodiments, in any one of Formulae (VIII) to (XI), R⁹ is furanyl, pyrrolyl, thienyl, imidazolyl, isoxazolyl, isothiazolyl, oxazolyl, pyrazolyl, thiazolyl, 1,2,4-oxadiazolyl, 1,2,3-triazolyl, pyridinyl, pyridazinyl, pyrimidinyl, pyrazinyl, imidazo[2,1-b]-thiazolyl, pyrrolo[2,1-b]thiazolyl, pyrazolo[1,5-b][1,2,4]triazolyl, 2,4,5,6-tetrahydrocyclopenta-[c]pyrrolyl, 2,4,5,6-tetrahydrocyclopenta[c]pyrazolyl, thieno[3,2-b]pyrrolyl, benzo[b]thienyl, 2,3-dihydrobenzofuranyl, 6,7-dihydro-5H-pyrazolo[5,1-b][1,3]oxazinyl, 6,7-dihydro-4H-pyrazolo[5,1-c][1,4]oxazinyl, 1,4-dihydropyrrolo[1,2-b]pyridazinyl, imidazo[1,2-a]pyridinyl, imidazo[1,2-a]pyridinyl, indazolyl, indolinyl, 4,5,6,7-tetrahydrobenzo[b]thienyl, 4,5,6,7-tetrahydroindazolyl, 4,5,6,7-tetrahydropyrazolo[1,5-a]pyrazinyl, 3,4-dihydro-2H-benzo[b][1,4]-oxazinyl, or chromenyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a}S(O)₂R^{1d}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (VIII) to (XI), R⁹ is furan-2-yl, furan-3-yl, pyrrol-2-yl, pyrrol-3-yl, thien-2-yl, thien-3-yl, imidazol-4-yl, isoxazol-4-yl, isoxazol-5-yl, isothiazol-5-yl, oxazol-2-yl, oxazol-4-yl, oxazol-5-yl, pyrazol-3-yl, pyrazol-4-yl, pyrazol-5-yl, thiazol-2-yl, thiazol-4-yl, thiazol-5-yl, 1,2,4-oxadiazol-3-yl, 1,2,3-triazol-4-yl, pyridin-2-yl, pyridin-3-yl, pyridin-4-yl, pyridazin-3-yl, pyrimidin-4-yl, imidazo[2,1-b]thiazol-5-yl, pyrrolo-[2,1-b]thiazol-6-yl, 2,4,5,6-tetrahydrocyclopenta[c]pyrrol-1-yl, 2,4,5,6-tetrahydrocyclopenta[c]-pyrazol-3-yl, thieno[3,2-b]pyrrol-5-yl, benzo[b]thien-2-yl, 2,3-dihydrobenzofuran-5-yl, 2,3-dihydrobenzofuran-7-yl, 6,7-dihydro-5H-pyrazolo[5,1-b][1,3]oxazin-2-yl, 6,7-dihydro-4H-pyrazolo[5,1-c][1,4]oxazin-2-yl, 1,4-dihydropyrrolo[1,2-b]pyridazin-3-yl, imidazo[1,2-a]-pyridin-3-yl, imidazo[1,2-a]pyridin-6-yl, indazol-3-yl, indazol-5-yl, indolin-6-yl, pyrrolo[2,3-b]-pyridin-3-yl, pyrazolo[1,5-b][1,2,4]triazol-7-yl, 4,5,6,7-tetrahydrobenzo[b]thien-2-yl, 4,5,6,7-tetrahydrobenzo[b]thien-3-yl, 4,5,6,7-tetrahydroindazol-3-yl, 4,5,6,7-tetrahydropyrazolo[1,5-a]-pyrazin-2-yl, 3,4-dihydro-2H-benzo[b][1,4]oxazin-6-yl, chromen-2-yl, or chromen-3-yl, each optionally substituted with one, two, or three substituents, each of which is independently cyano, fluoro, chloro, oxo, methyl, difluoromethyl, trifluoromethyl, ethyl, isopropyl, tert-butyl, phenyl, 2-fluorophenyl, 5-chloro-1,2,4-thiadiazol-3-yl, pyridin-3-yl, carboxy, methoxycarbonyl, ethoxy-carbonyl, aminocarbonyl, ethylaminocarbonyl, isopropylaminocarbonyl, hydroxyl, methoxy, ethoxy, amino, cyclopropylamino, dimethylamino, or mercapto.

In certain embodiments, in any one of Formulae (VIII) to (XI), R⁹ is 4-cyano-furan-2-yl, 5-chlorofuran-2-yl, 2-methyl-4-trifluoromethylfuran-3-yl, 3-cyanopyrrol-2-yl, 4-cyanopyrrol-2-yl, 1-methylpyrrol-2-yl, 5-carboxypyrrol-2-yl, 2-ethoxycarbonylpyrrol-4-yl, 2-ethoxycarbonylpyrrol-5-yl, 5-aminocarbonylpyrrol-2-yl, 1-methyl-5-trifluoromethylpyrrol-2-yl, thien-2-yl, thien-3-yl, 5-methylthien-2-yl, 5-(5-chloro-1,2,4-thiadiazol-3-yl) thien-2-yl, 3-methoxythien-2-yl, 2,3-dimethylthien-4-yl, 1-methylimidazol-4-yl, 2-methylimidazol-4-yl, 5-(pyridin-3-yl) imidazol-4-yl, 1,2-dimethylimidazol-5-yl, 1-methyl-5-trfluoromethylimidazol-4-yl, 2-mercapto-1-methylimidazol-5-yl, 3-methylisoxazol-5-yl, 3,5-dimethylisoxazol-4-yl, 3-methyl-isothiazol-5-yl, 4-amino-3-aminocarbonylisothiazol-5-yl, 4-amino-3-(ethylaminocarbonyl)-isothiazol-5-yl, 4-amino-3-(isopropylaminocarbonyl) isothiazol-5-yl, oxazol-2-yl, oxazol-4-yl, 5-phenyloxazol-4-yl, 2,4-dimethyloxazol-5-yl, 2,5-dimethyloxazol-4-yl, pyrazol-4-yl, 1-methyl-pyrazol-4-yl, 1-ethylpyrazol-4-yl, 1-isopropylpyrazol-5-yl, 5-isopropylpyrazol-3-yl, 1-tert-butyl-pyrazol-3-yl, 1-(2-fluorophenyl) pyrazol-4-yl, 5-(pyridin-3-yl) pyrazol-3-yl, 1,5-dimethylpyrazol-3-yl, 1,5-dimethylpyrazol-4-yl, 1-ethyl-3-methylpyrazol-5-yl, 1-methyl-3-trifluoromethyl-pyrazol-4-yl, 1-methyl-3-methoxycarbonylpyrazol-5-yl, 4-amino-1-methylpyrazol-3-yl, 4-amino-1-methylpyrazol-5-yl, 5-amino-1-phenylpyrazol-4-yl, 1,3,5-trimethylpyrazol-4-yl, 1-methyl-3-difluoromethyl-5-fluoropyrazol-4-yl, thiazol-4-yl, thiazol-5-yl, 2-cyanothiazol-5-yl, 4-methylthiazol-2-yl, 5-methylthiazol-2-yl, 2-phenylthiazol-4-yl, 2-aminothiazol-4-yl, 2-(cyclo-propylamino) thiazol-5-yl, 2,4-dimethylthiazol-5-yl, 5-amino-2-methylthiazol-4-yl, 2-amino-4-methylthiazol-5-yl, 1,2,4-oxadiazol-3-yl, 4-amino-1,2,5-oxadiazol-3-yl, 1-(tert-butyl)-1,2,3-triazol-4-yl, 5-cyanopyridin-2-yl, 2-isopropylpyridin-4-yl, 5-methoxypyridin-2-yl, 6-ethoxy-pyridin-2-yl, 2-chloro-5-methylpyridin-3-yl, 5-cyanopyrazin-2-yl, pyridazin-3-yl, 4,6-dihydroxy-pyridazin-3-yl, pyrimidin-4-yl, 2-(dimethylamino)pyrimidin-4-yl, imidazo[2,1-b]thiazol-5-yl, pyrrolo[2,1-b]thiazol-6-yl, 2,4,5,6-tetrahydrocyclopenta[c]pyrrol-1-yl, 2-methyl-2,4,5,6-tetrahydrocyclopenta[c]pyrazol-3-yl, thieno[3,2-b]pyrrol-5-yl, 6-methylbenzo[b]thien-2-yl, 2,3-dihydrobenzofuran-7-yl, 2,2,4,6,7-pentamethyl-2,3-dihydrobenzofuran-5-yl, 6,7-dihydro-5H-pyrazolo[5,1-b][1,3]oxazin-2-yl, 6,7-dihydro-4H-pyrazolo[5,1-c][1,4]oxazin-2-yl, 4-oxo-1,4-dihydropyrrolo[1,2-b]pyridazin-3-yl, 1-methyl-4-oxo-1,4-dihydropyrrolo[1,2-b]pyridazin-3-yl, imidazo[1,2-a]pyridin-3-yl, imidazo[1,2-a]pyridin-6-yl, indazol-5-yl, 6-methoxyindazol-3-yl, 6-methoxyindazol-3-yl, 2-oxoindolin-6-yl, pyrrolo[2,3-b]pyridin-3-yl, 2-methyl-3H-pyrazolo[1,5-b][1,2,4]triazol-7-yl, 4,5,6,7-tetrahydrobenzo[b]thien-2-yl, 4,5,6,7-tetrahydrobenzo[b]thien-3-yl, 4,5,6,7-tetrahydroindazol-3-yl, 4-oxo-4,5,6,7-tetrahydropyrazolo[1,5-a]pyrazin-2-yl, 3-oxo-3,4-dihydro-2H-benzo[b][1,4]oxazin-6-yl, 4-oxochromen-2-yl, or 4-oxochromen-3-yl.

In certain embodiments, in any one of Formulae (VIII) to (XI), R⁹ is heterocyclyl, optionally substituted with one, two, or three substituents Q. In certain embodiments, in any one of Formulae (VIII) to (XI), R⁹ is monocyclic heterocyclyl, optionally substituted with one, two, or three substituents Q. In certain embodiments, in any one of Formulae (VIII) to (XI), R⁹ is 3-, 4-, 5-, 6-, or 7-membered heterocyclyl, each optionally substituted with one, two, or three substituents Q. In certain embodiments, in any one of Formulae (VIII) to (XI), R⁹ is 3-membered heterocyclyl, optionally substituted with one, two, or three substituents Q. In certain embodiments, in any one of Formulae (VIII) to (XI), R⁹ is 4-membered heterocyclyl, optionally substituted with one, two, or three substituents Q. In certain embodiments, in any one of Formulae (VIII) to (XI), R⁹ is 5-membered heterocyclyl, optionally substituted with one, two, or three substituents Q. In certain embodiments, in any one of Formulae (VIII) to (XI), R⁹ is 6-membered heterocyclyl, optionally substituted with one, two, or three substituents Q. In certain embodiments, in any one of Formulae (VIII) to (XI), R⁹ is 7-membered heterocyclyl, optionally substituted with one, two, or three substituents Q. In certain embodiments, in any one of Formulae (VIII) to (XI), R⁹ is bicyclic heterocyclyl, optionally substituted with one, two, or three substituents Q. In certain embodiments, in any one of Formulae (VIII) to (XI), R⁹ is bridged, fused, or spiro heterocyclyl, each optionally substituted with one, two, or three substituents Q. In certain embodiments, in any one of Formulae (VIII) to (XI), R⁹ is fused heterocyclyl, optionally substituted with one, two, or three substituents Q. In certain embodiments, in any one of Formulae (VIII) to (XI), R⁹ is bridged heterocyclyl, optionally substituted with one, two, or three substituents Q. In certain embodiments, in any one of Formulae (VIII) to (XI), R⁹ is spiro heterocyclyl, optionally substituted with one, two, or three substituents Q.

In certain embodiments, in any one of Formulae (VIII) to (XI), R⁹ is azetidinyl, oxetanyl, thietanyl, tetrahydropyranyl, piperidinyl, tetrahydrothiopyranyl, or morpholinyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1d}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (VIII) to (XI), R⁹ is azetidin-3-yl, oxetan-3-yl, thietan-3-yl, tetrahydropyran-4-yl, piperidin-2-yl, piperidin-4-yl, morpholin-3-yl, or tetrahydrothiopyran-4-yl, each optionally substituted with one, two, or three substituents, each of which is independently cyano, oxo, methyl, benzyl, 6-(cyclopropylamino)pyrimidin-4-yl, acetyl, or carbamimidoyl. In certain embodiments, in any one of Formulae (VIII) to (XI), R⁹ is azetidin-3-yl, oxetan-3-yl, 1,1-dioxidothietan-3-yl, tetrahydropyran-4-yl, 4-cyanotetrahydropyran-4-yl, 1-methylpiperidin-2-yl, 1-acetylpiperidin-4-yl, 1-(6-(cyclopropylamino)pyrimidin-4-yl)piperidin-4-yl, 1-carbamimidoylpiperidin-4-yl, 4-benzyl-5-oxomorpholin-3-yl, or tetrahydrothiopyran-4-yl.

In certain embodiments, in any one of Formulae (VIII) to (XI), p is an integer of 0, 1, or 2. In certain embodiments, in any one of Formulae (VIII) to (XI), p is an integer of 0. In certain embodiments, in any one of Formulae (VIII) to (XI), p is an integer of 1. In certain embodiments, in any one of Formulae (VIII) to (XI), p is an integer of 2.

In yet another embodiment, provided herein is a compound of Formula (XII):

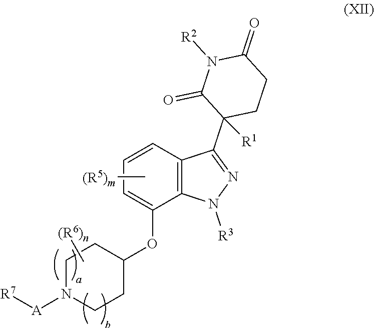

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, A, a, b, m, and n are each as defined herein.

In yet another embodiment, provided herein is a compound of Formula (XIII):

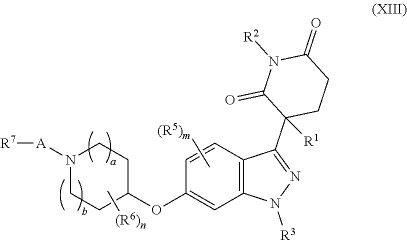

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, A, a, b, m, and n are each as defined herein.

In certain embodiments, in any one of Formulae (I) to (III), (XII), and (XIII), a and b are each independently an integer of 0, 1, or 2. In certain embodiments, in any one of Formulae (I) to (III), (XII), and (XIII), a and b are each independently an integer of 0 or 1. In certain embodiments, in any one of Formulae (I) to (III), (XII), and (XIII), a and b are each an integer of 1.

In yet another embodiment, provided herein is a compound of Formula (XIV):

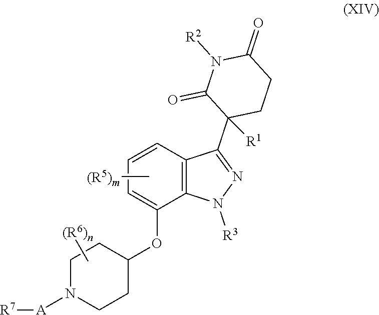

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, A, m, and n are each as defined herein.

In yet another embodiment, provided herein is a compound of Formula (XV):

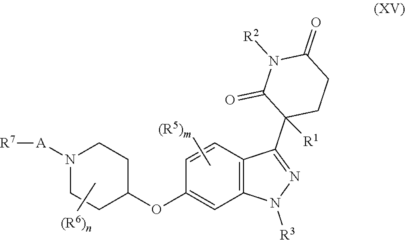

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, A, m, and n are each as defined herein.

In certain embodiments, in any one of Formulae (I) to (III) and (XII) to (XV), A is —C(O)—, —C(O)C(O)—, or —S(O₂)—. In certain embodiments, in any one of Formulae (I) to (III) and (XII) to (XV), A is —C(O)—or —C(O)C(O)—. In certain embodiments, in any one of Formulae (I) to (III) and (XII) to (XV), A is —C(O)—. In certain embodiments, in any one of Formulae (I) to (III) and (XII) to (XV), A is —C(O)C(O)—. In certain embodiments, in any one of Formulae (I) to (III) and (XII) to (XV), A is —S(O₂)—.

In yet another embodiment, provided herein is a compound of Formula (XVI):

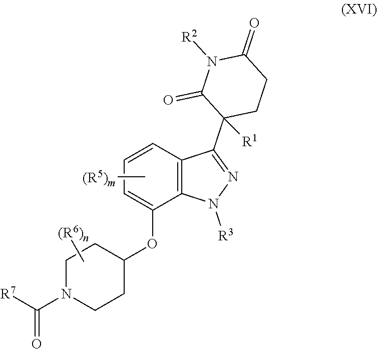

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, m, and n are each as defined herein.

In yet another embodiment, provided herein is a compound of Formula (XVII):

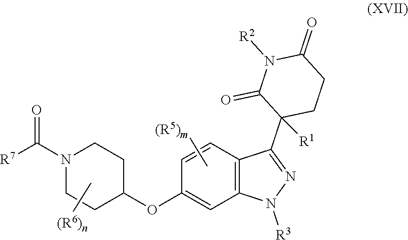

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, m, and n are each as defined herein.

In yet another embodiment, provided herein is a compound of Formula (XVIII):

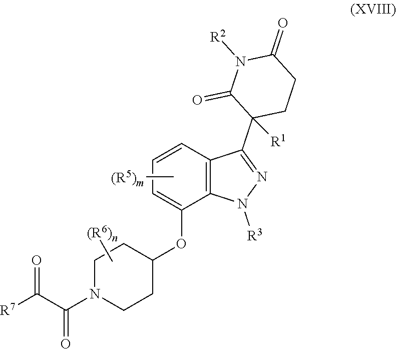

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, m, and n are each as defined herein.

In yet another embodiment, provided herein is a compound of Formula (XIX):

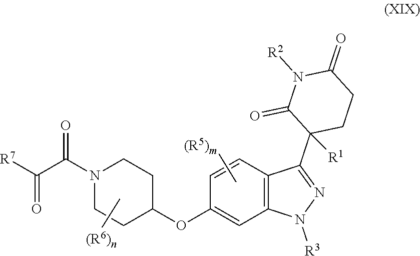

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁵, R⁶, R⁷, m, and n are each as defined herein.

In certain embodiments, in any one of Formulae (I) to (III) and (XII) to (XIX), R⁷ is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more substituents Q. In certain embodiments, in any one of Formulae (I) to (III) and (XII) to (XIX), R⁷ is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (I) to (III) and (XII) to (XIX), R⁷ is C₁₋₆ alkyl, optionally substituted with one, two, or three substituents Q. In certain embodiments, in any one of Formulae (I) to (III) and (XII) to (XIX), R⁷ is methyl, ethyl, propyl, or butyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III) and (XII) to (XIX), R⁷ is methyl, ethyl, propyl, or butyl, each optionally substituted with one, two, or three substituents, each of which is independently fluoro, cyclopropyl, cyclobutyl, 4-fluorophenyl, 5-cyano-1-methylpyrrol-2-yl, pyrazol-1-yl, thien-2-yl, 2-mercapto-4-methylthiazol-5-yl, 4-oxo-2-thioxothiazolidin-3-yl, 1-methylpiperidin-4-yl, 1,1-dioxidothiomorpholino, piperazin-1-yl, hydroxyl, 2-cyanophenoxy, benzoxy, 2-benzoxy, methylamino, phenylacetamido, guanidino, phenylthio, 4-chlorophenylthio, p-tolylthio, 2,4-difluorophenylthio, or phenylsulfonyl.

In certain embodiments, in any one of Formulae (I) to (III) and (XII) to (XIX), R⁷ is cyclopropylmethyl, cyclobutylmethyl, (4-fluorophenyl)difluoromethyl, (5-cyano-1-methyl-pyrrol-2-yl)methyl, pyrazol-1-ylmethyl, thien-2-ylmethyl, (2-mercapto-4-methylthiazol-5-yl)-methyl, (4-oxo-2-thioxothiazolidin-3-yl)methyl, (1-methylpiperidin-4-yl)methyl, (1,1-dioxido-thiomorpholino)methyl, piperazin-1-ylmethyl, 2-hydroxyethyl, (2-cyanophenoxy)methyl, benzoxymethyl, 2-benzoxyprop-2-yl, methylaminomethyl, phenylacetamidomethyl, guanidino-methyl, 2-guanidinoethyl, phenylthiomethyl, (4-chlorophenylthio)methyl, (p-tolylthio)methyl, (2,4-difluorophenylthio)methyl, or 2-(phenylsulfonyl)ethyl.

In certain embodiments, in any one of Formulae (I) to (III) and (XII) to (XIX), R⁷ is C₂₋₆ alkenyl, optionally substituted with one, two, or three substituents Q. In certain embodiments, in any one of Formulae (I) to (III) and (XII) to (XIX), R⁷ is ethenyl, propenyl, or butenyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III) and (XII) to (XIX), R⁷ is ethenyl, propenyl, or butenyl, each optionally substituted with one, two, or three substituents, each of which is independently phenyl, 4-trifluoromethylphenyl, 3,4-difluorophenyl, 4-aminophenyl, 5-methyl-thien-2-yl, thiazol-2-yl, benzofuran-2-yl, or benzo[d][1,3]dioxol-5-yl. In certain embodiments, in any one of Formulae (I) to (III) and (XII) to (XIX), R⁷ is 1-butenyl, 2-phenylethenyl, 2-(4-trifluoromethylphenyl)ethenyl, 2-(3,4-difluorophenyl)ethenyl, 2-(4-aminophenyl)ethenyl, 2-(5-methylthien-2-yl)ethenyl, 2-(thiazol-2-yl)ethenyl, 2-(benzofuran-2-yl)ethenyl, or 2-(benzo[d]-[1,3]dioxol-5-yl)ethenyl.

In certain embodiments, in any one of Formulae (I) to (III) and (XII) to (XIX), R⁷ is C₂₋₆ alkynyl, optionally substituted with one, two, or three substituents Q. In certain embodiments, in any one of Formulae (I) to (III) and (XII) to (XIX), R⁷ is ethynyl, propynyl, or butynyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III) and (XII) to (XIX), R⁷ is 1-butynyl, phenyl-ethynyl, (3-fluorophenyl)ethynyl, or (4-trifluoromethylphenyl)ethynyl.

In certain embodiments, in any one of Formulae (I) to (III) and (XII) to (XIX), R⁷ is C₃₋₁₀ cycloalkyl, optionally substituted with one, two, or three substituents Q. In certain embodiments, in any one of Formulae (I) to (III) and (XII) to (XIX), R⁷ is monocyclic C₃₋₁₀ cycloalkyl, optionally substituted with one, two, or three substituents Q. In certain embodiments, in any one of Formulae (I) to (III) and (XII) to (XIX), R⁷ is cyclopropyl, cyclobutyl, cyclopentyl, cyclohexyl, or cycloheptyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a}S(O)₂R^{1b}, —SR^{1a}, —S(O)₂R^{1a}, or —S(O)₂NR^{1b}R^{1c}, wherein each R^{1a}, R^{1b}, R^{1a}, and R^{1d} is as defined herein. In certain embodiments, in any one of Formulae (I) to (III) and (XII) to (XIX), R⁷ is bicyclic C₄₋₁₀ cycloalkyl, optionally substituted with one, two, or three substituents Q. In certain embodiments, in any one of Formulae (I) to (III) and (XII) to (XIX), R⁷ is bridged, fused, or spiro C₄₋₁₀ cycloalkyl, each optionally substituted with one, two, or three substituents Q. In certain embodiments, in any one of Formulae (I) to (III) and (XII) to (XIX), R⁷ is bridged C₄₋₁₀ cycloalkyl, optionally substituted with one, two, or three substituents Q. In certain embodiments, in any one of Formulae (I) to (III) and (XII) to (XIX), R⁷ is fused C₄₋₁₀ cycloalkyl, optionally substituted with one, two, or three substituents Q. In certain embodiments, in any one of Formulae (I) to (III) and (XII) to (XIX), R⁷ is spiro C₄₋₁₀ cycloalkyl, optionally substituted with one, two, or three substituents Q.

In certain embodiments, in any one of Formulae (I) to (III) and (XII) to (XIX), R⁷ is cyclopropyl, cyclobutyl, cyclobut-1-ene-1-yl, cyclopentyl, cyclohexyl, or bicyclo[1.1.1]pentyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III) and (XII) to (XIX), R⁷ is cyclopropyl, cyclo-butyl, cyclobut-1-ene-1-yl, cyclopentyl, cyclohexyl, or bicyclo[1.1.1]pent-1-yl, each optionally substituted with one, two, or three substituents, each of which is independently cyano, hydroxy-methyl, tert-butyl, phenyl, or hydroxyl. In certain embodiments, in any one of Formulae (I) to (III) and (XII) to (XIX), R⁷ is 1-(hydroxymethyl)cyclopropyl, 1-hydroxycyclopropyl, cyclobutyl, 1-cyanocyclobutyl, 3-phenylcyclobutyl, cyclobut-1-ene-1-yl, cyclopentyl, cyclohexyl, 4-hydroxycyclohexyl, bicyclo[1.1.1]pent-1-yl, 3-cyanobicyclo[1.1.1]pent-1-yl, or 3-(tert-butyl)-bicyclo[1.1.1]pent-1-yl.

In certain embodiments, in any one of Formulae (I) to (III) and (XII) to (XIX), R⁷ is C₆₋₁₄ aryl, optionally substituted with one, two, or three substituents Q. In certain embodiments, in any one of Formulae (I) to (III) and (XII) to (XIX), R⁷ is phenyl, optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III) and (XII) to (XIX), R⁷ is bicyclic C₈₋₁₄ aryl, optionally substituted with one, two, or three substituents Q. In certain embodiments, in any one of Formulae (I) to (III) and (XII) to (XIX), R⁷ is phenyl, optionally substituted with one, two, or three substituents, each of which is independently cyano, fluoro, nitro, phenylthiomethyl, methyl, tert-butyl, 4-fluorophenyl, imidazol-2-yl, pyrazol-1-yl, 5-methyl-1,2,4-oxadiazol-3-yl, hydroxyl, methoxy, trifluoro-methoxy, isopropoxy, phenoxy, aminocarbonyl, amino, or phenylsulfonamido.

In certain embodiments, in any one of Formulae (I) to (III) and (XII) to (XIX), R⁷ is 4-cyanophenyl, 4-fluorophenyl, 4-nitrophenyl, 2-(phenylthiomethyl)phenyl, 4-(ter-butyl)-phenyl, 3-(4-fluorophenyl)phenyl, 4-(imidazol-2-yl)phenyl, 4-(pyrazol-1-yl)phenyl, 3-(5-methyl-1,2,4-oxadiazol-3-yl)phenyl, 2-methoxyphenyl, 3-methoxyphenyl, 4-methoxyphenyl, 2-trifluoro-methoxyphenyl, 3-trifluoromethoxyphenyl, 4-trifluoromethoxyphenyl, 3-isopropoxyphenyl, 4-isopropoxyphenyl, 3-phenoxyphenyl, 4-phenoxyphenyl, 4-aminocarbonylphenyl, 4-phenyl-sulfonamidophenyl, 4-cyano-2-fluorophenyl, 3-cyano-5-methylphenyl, 3-hydroxy-4-methoxy-phenyl, 4-hydroxy-3-trifluoromethoxyphenyl, or 3-amino-5-cyanophenyl.

In certain embodiments, in any one of Formulae (I) to (III) and (XII) to (XIX), R⁷ is heteroaryl, optionally substituted with one, two, or three substituents Q. In certain embodiments, in any one of Formulae (I) to (III) and (XII) to (XIX), R⁷ is monocyclic heteroaryl, optionally substituted with one, two, or three substituents Q. In certain embodiments, in any one of Formulae (I) to (III) and (XII) to (XIX), R⁷ is 5- or 6-membered heteroaryl, each optionally substituted with one, two, or three substituents Q. In certain embodiments, in any one of Formulae (I) to (III) and (XII) to (XIX), R⁷ is 5-membered heteroaryl, optionally substituted with one, two, or three substituents Q. In certain embodiments, in any one of Formulae (I) to (III) and (XII) to (XIX), R⁷ is 6-membered heteroaryl, optionally substituted with one, two, or three substituents Q. In certain embodiments, in any one of Formulae (I) to (III) and (XII) to (XIX), R⁷ is bicyclic heteroaryl, optionally substituted with one, two, or three substituents Q. In certain embodiments, in any one of Formulae (I) to (III) and (XII) to (XIX), R⁷ is 5,5-, 5,6, or 6,6-fused heteroaryl, each optionally substituted with one, two, or three substituents Q. In certain embodiments, in any one of Formulae (I) to (III) and (XII) to (XIX), R⁷ is 5,5-fused heteroaryl, optionally substituted with one, two, or three substituents Q. In certain embodiments, in any one of Formulae (I) to (III) and (XII) to (XIX), R⁷ is 5,6-fused heteroaryl, optionally substituted with one, two, or three substituents Q. In certain embodiments, in any one of Formulae (I) to (III) and (XII) to (XIX), R⁷ is 6,6-fused heteroaryl, optionally substituted with one, two, or three substituents Q.

In certain embodiments, in any one of Formulae (I) to (III) and (XII) to (XIX), R⁷ is furanyl, pyrrolyl, thienyl, imidazolyl, isoxazolyl, isothiazolyl, oxazolyl, pyrazolyl, thiazolyl, 1,2,4-oxadiazolyl, 1,2,3-triazolyl, pyridinyl, pyridazinyl, pyrimidinyl, pyrazinyl, imidazo[2,1-b]-thiazolyl, pyrrolo[2,1-b]thiazolyl, pyrazolo[1,5-b][1,2,4]triazolyl, 2,4,5,6-tetrahydrocyclopenta-[c]pyrrolyl, 2,4,5,6-tetrahydrocyclopenta[c]pyrazolyl, thieno[3,2-b]pyrrolyl, benzo[b]thienyl, 2,3-dihydrobenzofuranyl, 6,7-dihydro-5H-pyrazolo[5,1-b][1,3]oxazinyl, 6,7-dihydro-4H-pyrazolo[5,1-c][1,4]-oxazinyl, 1,4-dihydropyrrolo[1,2-b]pyridazinyl, imidazo[1,2-a]pyridinyl, imidazo[1,2-a]pyridinyl, indazolyl, indolinyl, 4,5,6,7-tetrahydrobenzo[b]thienyl, 4,5,6,7-tetrahydroindazolyl, 4,5,6,7-tetrahydropyrazolo[1,5-a]pyrazinyl, 3,4-dihydro-2H-benzo[b][1,4]-oxazinyl, or chromenyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (I) to (III) and (XII) to (XIX), R⁷ is furan-2-yl, furan-3-yl, pyrrol-2-yl, pyrrol-3-yl, thien-2-yl, thien-3-yl, imidazol-4-yl, isoxazol-4-yl, isoxazol-5-yl, isothiazol-5-yl, oxazol-2-yl, oxazol-4-yl, oxazol-5-yl, pyrazol-3-yl, pyrazol-4-yl, pyrazol-5-yl, thiazol-2-yl, thiazol-4-yl, thiazol-5-yl, 1,2,4-oxadiazol-3-yl, 1,2,3-triazol-4-yl, pyridin-2-yl, pyridin-3-yl, pyridin-4-yl, pyridazin-3-yl, pyrimidin-4-yl, imidazo[2,1-b]thiazol-5-yl, pyrrolo-[2,1-b]thiazol-6-yl, 2,4,5,6-tetrahydrocyclopenta[c]pyrrol-1-yl, 2,4,5,6-tetrahydro-cyclopenta[c]pyrazol-3-yl, thieno[3,2-b]pyrrol-5-yl, benzo[b]thien-2-yl, 2,3-dihydrobenzofuran-5-yl, 2,3-dihydrobenzofuran-7-yl, 6,7-dihydro-5H-pyrazolo[5,1-b][1,3]oxazin-2-yl, 6,7-dihydro-4H-pyrazolo[5,1-c][1,4]oxazin-2-yl, 1,4-dihydropyrrolo[1,2-b]pyridazin-3-yl, imidazo[1,2-a]-pyridin-3-yl, imidazo[1,2-a]pyridin-6-yl, indazol-3-yl, indazol-5-yl, indolin-6-yl, pyrrolo[2,3-b]-pyridin-3-yl, pyrazolo[1,5-b][1,2,4]triazol-7-yl, 4,5,6,7-tetrahydrobenzo[b]thien-2-yl, 4,5,6,7-tetrahydrobenzo[b]thien-3-yl, 4,5,6,7-tetrahydroindazol-3-yl, 4,5,6,7-tetrahydropyrazolo[1,5-a]-pyrazin-2-yl, 3,4-dihydro-2H-benzo[b][1,4]oxazin-6-yl, chromen-2-yl, or chromen-3-yl, each optionally substituted with one, two, or three substituents, each of which is independently cyano, fluoro, chloro, oxo, methyl, difluoromethyl, trifluoromethyl, ethyl, isopropyl, tert-butyl, phenyl, 2-fluorophenyl, 5-chloro-1,2,4-thiadiazol-3-yl, pyridin-3-yl, carboxy, methoxycarbonyl, ethoxy-carbonyl, aminocarbonyl, ethylaminocarbonyl, isopropylaminocarbonyl, hydroxyl, methoxy, ethoxy, amino, cyclopropylamino, dimethylamino, or mercapto.

In certain embodiments, in any one of Formulae (I) to (III) and (XII) to (XIX), R⁷ is 4-cyanofuran-2-yl, 5-chlorofuran-2-yl, 2-methyl-4-trifluoromethylfuran-3-yl, 3-cyanopyrrol-2-yl, 4-cyanopyrrol-2-yl, 1-methylpyrrol-2-yl, 5-carboxypyrrol-2-yl, 2-ethoxycarbonylpyrrol-4-yl, 2-ethoxycarbonylpyrrol-5-yl, 5-aminocarbonylpyrrol-2-yl, 1-methyl-5-trifluoromethylpyrrol-2-yl, thien-2-yl, thien-3-yl, 5-methylthien-2-yl, 5-(5-chloro-1,2,4-thiadiazol-3-yl) thien-2-yl, 3-methoxythien-2-yl, 2,3-dimethylthien-4-yl, 1-methylimidazol-4-yl, 2-methylimidazol-4-yl, 5-(pyridin-3-yl) imidazol-4-yl, 1,2-dimethylimidazol-5-yl, 1-methyl-5-trfluoromethylimidazol-4-yl, 2-mercapto-1-methylimidazol-5-yl, 3-methylisoxazol-5-yl, 3,5-dimethylisoxazol-4-yl, 3-methyl-isothiazol-5-yl, 4-amino-3-aminocarbonylisothiazol-5-yl, 4-amino-3-(ethylaminocarbonyl)-isothiazol-5-yl, 4-amino-3-(isopropylaminocarbonyl) isothiazol-5-yl, oxazol-2-yl, oxazol-4-yl, 5-phenyloxazol-4-yl, 2,4-dimethyloxazol-5-yl, 2,5-dimethyloxazol-4-yl, pyrazol-4-yl, 1-methyl-pyrazol-4-yl, 1-ethylpyrazol-4-yl, 1-isopropylpyrazol-5-yl, 5-isopropylpyrazol-3-yl, 1-tert-butyl-pyrazol-3-yl, 1-(2-fluorophenyl) pyrazol-4-yl, 5-(pyridin-3-yl) pyrazol-3-yl, 1,5-dimethylpyrazol-3-yl, 1,5-dimethylpyrazol-4-yl, 1-ethyl-3-methylpyrazol-5-yl, 1-methyl-3-trifluoromethyl-pyrazol-4-yl, 1-methyl-3-methoxycarbonylpyrazol-5-yl, 4-amino-1-methylpyrazol-3-yl, 4-amino-1-methylpyrazol-5-yl, 5-amino-1-phenylpyrazol-4-yl, 1,3,5-trimethylpyrazol-4-yl, 1-methyl-3-difluoromethyl-5-fluoropyrazol-4-yl, thiazol-4-yl, thiazol-5-yl, 2-cyanothiazol-5-yl, 4-methylthiazol-2-yl, 5-methylthiazol-2-yl, 2-phenylthiazol-4-yl, 2-aminothiazol-4-yl, 2-(cyclo-propylamino) thiazol-5-yl, 2,4-dimethylthiazol-5-yl, 5-amino-2-methylthiazol-4-yl, 2-amino-4-methylthiazol-5-yl, 1,2,4-oxadiazol-3-yl, 4-amino-1,2,5-oxadiazol-3-yl, 1-(tert-butyl)-1,2,3-triazol-4-yl, 5-cyanopyridin-2-yl, 2-isopropylpyridin-4-yl, 5-methoxypyridin-2-yl, 6-ethoxy-pyridin-2-yl, 2-chloro-5-methylpyridin-3-yl, 5-cyanopyrazin-2-yl, pyridazin-3-yl, 4,6-dihydroxy-pyridazin-3-yl, pyrimidin-4-yl, 2-(dimethylamino)pyrimidin-4-yl, imidazo[2,1-b]thiazol-5-yl, pyrrolo[2,1-b]thiazol-6-yl, 2,4,5,6-tetrahydrocyclopenta[c]pyrrol-1-yl, 2-methyl-2,4,5,6-tetrahydrocyclopenta[c]pyrazol-3-yl, thieno[3,2-b]pyrrol-5-yl, 6-methylbenzo[b]thien-2-yl, 2,3-dihydrobenzofuran-7-yl, 2,2,4,6,7-pentamethyl-2,3-dihydrobenzofuran-5-yl, 6,7-dihydro-5H-pyrazolo[5,1-b][1,3]oxazin-2-yl, 6,7-dihydro-4H-pyrazolo[5,1-c][1,4]oxazin-2-yl, 4-oxo-1,4-dihydropyrrolo[1,2-b]pyridazin-3-yl, 1-methyl-4-oxo-1,4-dihydropyrrolo[1,2-b]pyridazin-3-yl, imidazo[1,2-a]pyridin-3-yl, imidazo[1,2-a]pyridin-6-yl, indazol-5-yl, 6-methoxyindazol-3-yl, 6-methoxyindazol-3-yl, 2-oxoindolin-6-yl, pyrrolo[2,3-b]pyridin-3-yl, 2-methyl-3H-pyrazolo[1,5-b][1,2,4]triazol-7-yl, 4,5,6,7-tetrahydrobenzo[b]thien-2-yl, 4,5,6,7-tetrahydrobenzo[b]thien-3-yl, 4,5,6,7-tetrahydroindazol-3-yl, 4-oxo-4,5,6,7-tetrahydropyrazolo[1,5-a]pyrazin-2-yl, 3-oxo-3,4-dihydro-2H-benzo[b][1,4]oxazin-6-yl, 4-oxochromen-2-yl, or 4-oxochromen-3-yl.

In certain embodiments, in any one of Formulae (I) to (III) and (XII) to (XIX), R⁷ is heterocyclyl, optionally substituted with one, two, or three substituents Q. In certain embodiments, in any one of Formulae (I) to (III) and (XII) to (XIX), R⁷ is monocyclic heterocyclyl, optionally substituted with one, two, or three substituents Q. In certain embodiments, in any one of Formulae (I) to (III) and (XII) to (XIX), R⁷ is 3-, 4-, 5-, 6-, or 7-membered heterocyclyl, each optionally substituted with one, two, or three substituents Q. In certain embodiments, in any one of Formulae (I) to (III) and (XII) to (XIX), R⁷ is 3-membered heterocyclyl, optionally substituted with one, two, or three substituents Q. In certain embodiments, in any one of Formulae (I) to (III) and (XII) to (XIX), R⁷ is 4-membered heterocyclyl, optionally substituted with one, two, or three substituents Q. In certain embodiments, in any one of Formulae (I) to (III) and (XII) to (XIX), R⁷ is 5-membered heterocyclyl, optionally substituted with one, two, or three substituents Q. In certain embodiments, in any one of Formulae (I) to (III) and (XII) to (XIX), R⁷ is 6-membered heterocyclyl, optionally substituted with one, two, or three substituents Q. In certain embodiments, in any one of Formulae (I) to (III) and (XII) to (XIX), R⁷ is 7-membered heterocyclyl, optionally substituted with one, two, or three substituents Q. In certain embodiments, in any one of Formulae (I) to (III) and (XII) to (XIX), R⁷ is bicyclic heterocyclyl, optionally substituted with one, two, or three substituents Q. In certain embodiments, in any one of Formulae (I) to (III) and (XII) to (XIX), R⁷ is bridged, fused, or spiro heterocyclyl, each optionally substituted with one, two, or three substituents Q. In certain embodiments, in any one of Formulae (I) to (III) and (XII) to (XIX), R⁷ is bridged heterocyclyl, optionally substituted with one, two, or three substituents Q. In certain embodiments, in any one of Formulae (I) to (III) and (XII) to (XIX), R⁷ is fused heterocyclyl, optionally substituted with one, two, or three substituents Q. In certain embodiments, in any one of Formulae (I) to (III) and (XII) to (XIX), R⁷ is spiro heterocyclyl, optionally substituted with one, two, or three substituents Q.

In certain embodiments, in any one of Formulae (I) to (III) and (XII) to (XIX), R⁷ is azetidinyl, oxetanyl, thietanyl, tetrahydropyranyl, piperidinyl, tetrahydrothiopyranyl, or morpholinyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1d}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III) and (XII) to (XIX), R⁷ is azetidin-3-yl, oxetan-3-yl, thietan-3-yl, tetrahydropyran-4-yl, piperidin-2-yl, piperidin-4-yl, morpholin-3-yl, or tetrahydrothiopyran-4-yl, each optionally substituted with one, two, or three substituents, each of which is independently cyano, oxo, methyl, benzyl, 6-(cyclopropylamino)pyrimidin-4-yl, acetyl, or carbamimidoyl. In certain embodiments, in any one of Formulae (I) to (III) and (XII) to (XIX), R⁷ is azetidin-3-yl, oxetan-3-yl, 1,1-dioxidothietan-3-yl, tetrahydropyran-4-yl, 4-cyano-tetrahydropyran-4-yl, 1-methylpiperidin-2-yl, 1-acetylpiperidin-4-yl, 1-(6-(cyclopropylamino)-pyrimidin-4-yl)piperidin-4-yl, 1-carbamimidoylpiperidin-4-yl, 4-benzyl-5-oxomorpholin-3-yl, or tetrahydrothiopyran-4-yl.

In certain embodiments, in any one of Formulae (I) to (III) and (XII) to (XIX), n is an integer of 0, 1, or 2. In certain embodiments, in any one of Formulae (I) to (III) and (XII) to (XIX), n is an integer of 0. In certain embodiments, in any one of Formulae (I) to (III) and (XII) to (XIX), n is an integer of 1. In certain embodiments, in any one of Formulae (I) to (III) and (XII) to (XIX), n is an integer of 2.

In yet another embodiment, provided herein is a compound of Formula (XX):

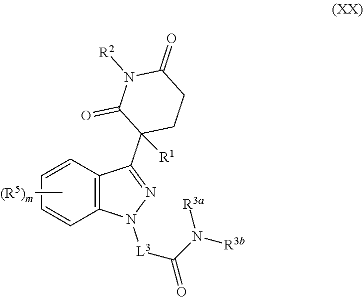

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R⁵, R^{3a}, R^{3b}, L³, and m are each as defined herein.

In certain embodiments, in Formula (I) or (XX), L³ is C₁₋₆ alkylene or C₁₋₆ heteroalkylene, each optionally substituted with one or more substituents Q. In certain embodiments, in Formula (I) or (XX), L³ is C₁₋₆ alkylene, optionally substituted with one, two, or three substituents Q. In certain embodiments, in Formula (I) or (XX), L³ is methanediyl, ethanediyl, or propanediyl, each optionally substituted with one, two, or three substituents Q. In certain embodiments, in Formula (I) or (XX), L³ is methanediyl, optionally substituted with one, two, or three substituents Q.

In still another embodiment, provided herein is a compound of Formula (XXI):

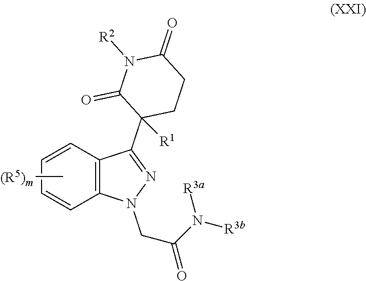

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R⁵, R^{3a}, R^{3b}, and m are each as defined herein.

In certain embodiments, in any one of Formulae (I) to (III), (XX), and (XXI), R^{3a} is hydrogen; or C₁₋₆ alkyl, optionally substituted with one or more substituents Q. In certain embodiments, in any one of Formulae (I) to (III), (XX), and (XXI), R^{3a} is hydrogen. In certain embodiments, in any one of Formulae (I) to (III), (XX), and (XXI), R^{3a} is C₁₋₆ alkyl, optionally substituted with one, two, or three substituents Q. In certain embodiments, in any one of Formulae (I) to (III), (XX), and (XXI), R^{3a} is methyl.

In certain embodiments, in any one of Formulae (I) to (III), (XX), and (XXI), R^{3b} is C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one or more substituents Q. In certain embodiments, in any one of Formulae (I) to (III), (XX), and (XXI), R^{3b} is C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (I) to (III), (XX), and (XXI), R^{3b} is C₁₋₆ alkyl, optionally substituted with one, two, or three substituents Q. In certain embodiments, in any one of Formulae (I) to (III), (XX), and (XXI), R^{3b} is methyl, ethyl, butyl, pentyl, or hexyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III), (XX), and (XXI), R^{3b} is methyl, ethyl, butyl, pentyl, or hexyl, each optionally substituted with one, two, or three substituents, each of which is independently cyano, cyclopropyl, 1-hydroxycyclopropyl, cyclopentyl, 1-cyanocyclopentyl, 3-hydroxycyclopentyl, 5-methylfuran-2-yl, 1-methylimidazol-5-yl, 5-methylisoxazol-3-yl, 1-methylpyrazol-3-yl, 1,5-dimethylpyrazol-3-yl, thien-2-yl, thiazol-2-yl, thiazol-4-yl, 2-chloro-thiazol-5-yl, 5-isopropyl-1,2,4-oxadiazol-3-yl, 3-cyclohexyl-1,2,4-oxadiazol-5-yl, 5-phenyl-1,2,4-oxadiazol-3-yl, 3,5-dimethyl-1,2,4-triazol-1-yl, pyridin-2-yl, 6-ethoxypyridin-3-yl, 1-methyl-2-oxo-1,2-dihydropyridin-4-yl, 1-methylindol-3-yl, 2-methyl-indol-5-yl, oxetan-2-yl, 5-oxopyrrolidin-2-yl, 1-methylpyrrolidin-3-yl, tetrahydrofuran-2-yl, 4-hydroxytetrahydropyran-4-yl, 4-benzylmorpholin-2-yl, 1,1-dioxidothiomorpholino, methyl-aminocarbonyl, cyclopentyloxy, p-tolyloxy, benzyloxy, or sulfamoyl.

In certain embodiments, in any one of Formulae (I) to (III), (XX), and (XXI), R^{3b} is neopentyl, 2-cyanoethyl, cyclopropylmethyl, (1-hydroxycyclopropyl)methyl, cyclopentyl-methyl, (1-cyanocyclopentyl)methyl, (3-hydroxycyclopentyl)methyl, 1-(5-methylfuran-2-yl)-propyl, thien-2-ylmethyl, (1-methylimidazol-5-yl)methyl, (5-methylisoxazol-3-yl)methyl, (1-methylpyrazol-3-yl)methyl, (1,5-dimethylpyrazol-3-yl)methyl, thiazol-2-ylmethyl, thiazol-4-yl-methyl, (2-chlorothiazol-5-yl)methyl, (3-methyl-1,2,4-oxadiazol-5-yl)methyl, 2-(5-isopropyl-1,2,4-oxadiazol-3-yl)ethyl, (3-cyclohexyl-1,2,4-oxadiazol-5-yl)methyl, (5-phenyl-1,2,4-oxadiazol-3-yl)methyl, 2-(3,5-dimethyl-1,2,4-triazol-1-yl)ethyl, 1-(pyridin-2-yl)ethyl, (6-ethoxy-pyridin-3-yl)methyl, (1-methyl-2-oxo-1,2-dihydropyridin-4-yl)methyl, (1-methylindol-3-yl)-methyl, (2-methylindol-5-yl)methyl, (oxetan-2-yl)methyl, (5-oxopyrrolidin-2-yl)methyl, (1-methylpyrrolidin-3-yl)methyl, (tetrahydrofuran-2-yl)methyl, (4-hydroxytetrahydropyran-4-yl)-methyl, (4-benzylmorpholin-2-yl)methyl, 3-(1,1-dioxidothiomorpholino) propyl, methylamino-carbonylmethyl, 2-(cyclopentyloxy)ethyl, 2-(p-tolyloxy)ethyl, 2-(benzyloxy)ethyl, or 2-sulfamoylethyl.

In certain embodiments, in any one of Formulae (I) to (III), (XX), and (XXI), R^{3b} is C₃₋₁₀ cycloalkyl, optionally substituted with one, two, or three substituents Q. In certain embodiments, in any one of Formulae (I) to (III), (XX), and (XXI), R^{3b} is monocyclic C₃₋₁₀ cycloalkyl, optionally substituted with one, two, or three substituents Q. In certain embodiments, in any one of Formulae (I) to (III), (XX), and (XXI), R^{3b} is bicyclic C₄₋₁₀ cycloalkyl, optionally substituted with one, two, or three substituents Q. In certain embodiments, in any one of Formulae (I) to (III), (XX), and (XXI), R^{3b} is bridged, fused, or spiro C₄₋₁₀ cycloalkyl, each optionally substituted with one, two, or three substituents Q. In certain embodiments, in any one of Formulae (I) to (III), (XX), and (XXI), R^{3b} is cyclopropyl, cyclobutyl, cyclopentyl, cyclo-hexyl, bicyclo[1.1.1]pentanyl, or bicyclo[2.2.2]octanyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III), (XX), and (XXI), R^{3b} is cyclopropyl, cyclobutyl, cyclopentyl, cyclohexyl, bicyclo[1.1.1]pentan-1-yl, or bicyclo-[2.2.2]octan-1-yl, each optionally substituted with one, two, or three substituents, each of which is independently cyano, fluoro, hydroxyl, or methoxy. In certain embodiments, in any one of Formulae (I) to (III), (XX), and (XXI), R^{3b} is cyclopropyl, cyclobutyl, cyclopentyl, 3-hydroxy-cyclopentyl, cyclohexyl, 4-hydroxycyclohexyl, 4-methoxycyclohexyl, 3-fluorobicyclo[1.1.1]-pentan-1-yl, or 4-cyanobicyclo[2.2.2]octan-1-yl.

In certain embodiments, in any one of Formulae (I) to (III), (XX), and (XXI), R^{3b} is C₆₋₁₄ aryl, optionally substituted with one, two, or three substituents Q. In certain embodiments, in any one of Formulae (I) to (III), (XX), and (XXI), R^{3b} is phenyl, optionally substituted with one, two, or three substituents Q. In certain embodiments, in any one of Formulae (I) to (III), (XX), and (XXI), R^{3b} is bicyclic C₈₋₁₄ aryl, optionally substituted with one, two, or three substituents Q. In certain embodiments, in any one of Formulae (I) to (III), (XX), and (XXI), R^{3b} is phenyl, 2,3-dihydroindenyl, or naphthalenyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III), (XX), and (XXI), R^{3b} is phenyl, 2,3-dihydroinden-1-yl, or naphthalen-1-yl, each optionally substituted with one, two, or three substituents, wherein each substituent is independently cyano, fluoro, chloro, bromo, methyl, trifluoromethyl, tert-butyl, (pyrrolidin-1-ylsulfonyl)methyl, pyrrol-1-yl, imidazol-1-yl, imidazol-2-yl, imidazol-5-yl, 2-methylimidazol-1-yl, oxazol-5-yl, 1-methyl-piperidin-4-yl, 4-methylpiperazin-1-yl, morpholino, carboxy, hydroxyl, methoxy, isopropoxy, trifluoromethoxy, p-tolyloxy, methyl-sulfonyl, or methylsulfamoyl.

In certain embodiments, in any one of Formulae (I) to (III), (XX), and (XXI), R^{3b} is 3-(tert-butyl)phenyl, 4-((pyrrolidin-1-ylsulfonyl)methyl)phenyl, 4-(pyrrol-1-yl)phenyl, 3-(imidazol-1-yl)phenyl, 4-(imidazol-2-yl)phenyl, 4-(imidazol-5-yl)phenyl, 4-(2-methylimidazol-1-yl)phenyl, 3-(oxazol-5-yl)phenyl, 4-(oxazol-5-yl)phenyl, 4-(1-methylpiperidin-4-yl)phenyl, 4-(4-methylpiperazin-1-yl)phenyl, 3-isopropoxyphenyl, 4-(trifluoromethoxy)phenyl, 4-(p-tolyl-oxy)phenyl, 4-(methylsulfonyl)phenyl, 4-(methylsulfamoyl)phenyl, 2-cyano-3-fluorophenyl, 2-cyano-6-fluorophenyl, 2-cyano-4-fluorophenyl, 5-chloro-2-cyanophenyl, 3-bromo-2-cyano-phenyl, 2-cyano-3-methylphenyl, 2-cyano-4-methylphenyl, 2-cyano-5-methylphenyl, 2-cyano-6-methylphenyl, 2-cyano-5-(trifluoromethyl)phenyl, 3-cyano-5-(trifluoromethyl)phenyl, 2-cyano-3-methoxyphenyl, 2-cyano-4-methoxyphenyl, 3-fluoro-5-morpholinophenyl, 2-cyano-4-carboxy-phenyl, 4-cyano-2-carboxyphenyl, 2-cyano-4,6-difluorophenyl, or 2-hydroxy-2,3-dihydroinden-1-yl, or 2-cyanonaphthalen-1-yl.

In certain embodiments, in any one of Formulae (I) to (III), (XX), and (XXI), R^{3b} is C₇₋₁₅ aralkyl, optionally substituted with one, two, or three substituents Q. In certain embodiments, in any one of Formulae (I) to (III), (XX), and (XXI), R^{3b} is monocyclic C₇₋₁₅ aralkyl, optionally substituted with one, two, or three substituents Q. In certain embodiments, in any one of Formulae (I) to (III), (XX), and (XXI), R^{3b} is bicyclic C₉₋₁₅ aralkyl, optionally substituted with one, two, or three substituents Q. In certain embodiments, in any one of Formulae (I) to (III), (XX), and (XXI), R^{3b} is benzyl, phenylethyl, or phenylpropyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III), (XX), and (XXI), R^{3b} is benzyl, phenylethyl, or phenylpropyl, each optionally substituted with one, two, or three substituents, each of which is independently cyano, fluoro, chloro, methyl, isopropyl, hydroxyl, methoxy, butoxy, or sulfamoyl. In certain embodiments, in any one of Formulae (I) to (III), (XX), and (XXI), R^{3b} is 2-cyanobenzyl, 3-cyanobenzyl, 4-isopropylbenzyl, 4-(morpholinomethyl)benzyl, 1-(4-fluoro-phenyl)ethyl, 1-(4-chlorophenyl)ethyl, 1-(p-tolyl)ethyl, 1-phenylpropyl, 4-(2-oxopyrrolidin-1-yl)benzyl, 4-hydroxybenzyl, 4-hydroxyphenethyl, 4-(sec-butoxy)benzyl, 2,4-difluorobenzyl, 2,5-difluorobenzyl, 3,5-dichlorobenzyl, 3,5-dimethoxybenzyl, or 4-sulfamoylphenethyl.

In certain embodiments, in any one of Formulae (I) to (III), (XX), and (XXI), R^{3b} is heteroaryl, optionally substituted with one, two, or three substituents Q. In certain embodiments, in any one of Formulae (I) to (III), (XX), and (XXI), R^{3b} is monocyclic heteroaryl, optionally substituted with one, two, or three substituents Q. In certain embodiments, in any one of Formulae (I) to (III), (XX), and (XXI), R^{3b} is 3-, 4-, 5-, 6-, or 7-membered heteroaryl, each optionally substituted with one, two, or three substituents Q. In certain embodiments, in any one of Formulae (I) to (III), (XX), and (XXI), R^{3b} is 3-membered heteroaryl, optionally substituted with one, two, or three substituents Q. In certain embodiments, in any one of Formulae (I) to (III), (XX), and (XXI), R^{3b} is 4-membered heteroaryl, optionally substituted with one, two, or three substituents Q. In certain embodiments, in any one of Formulae (I) to (III), (XX), and (XXI), R^{3b} is 5-membered heteroaryl, optionally substituted with one, two, or three substituents Q. In certain embodiments, in any one of Formulae (I) to (III), (XX), and (XXI), R^{3b} is 6-membered heteroaryl, optionally substituted with one, two, or three substituents Q. In certain embodiments, in any one of Formulae (I) to (III), (XX), and (XXI), R^{3b} is 7-membered heteroaryl, optionally substituted with one, two, or three substituents Q. In certain embodiments, in any one of Formulae (I) to (III), (XX), and (XXI), R^{3b} is bicyclic heteroaryl, optionally substituted with one, two, or three substituents Q. In certain embodiments, in any one of Formulae (I) to (III), (XX), and (XXI), R^{3b} is 5,5-, 5,6-, or 6,6-fused heteroaryl, each optionally substituted with one, two, or three substituents Q. In certain embodiments, in any one of Formulae (I) to (III), (XX), and (XXI), R^{3b} is 5,5-fused heteroaryl, optionally substituted with one, two, or three substituents Q. In certain embodiments, in any one of Formulae (I) to (III), (XX), and (XXI), R^{3b} is 5,6-fused heteroaryl, optionally substituted with one, two, or three substituents Q. In certain embodiments, in any one of Formulae (I) to (III), (XX), and (XXI), R^{3b} is 6,6-fused heteroaryl, optionally substituted with one, two, or three substituents Q.

In certain embodiments, in any one of Formulae (I) to (III), (XX), and (XXI), R^{3b} is pyrrolyl, thienyl, isoxazolyl, isothiazolyl, oxazolyl, pyrrolyl, pyrazolyl, thiazolyl, 1,2,4-triazolyl, 1,2,5-oxadiazolyl, 1,3,4-oxadiazolyl, 1,3,4-thiadiazolyl, pyridinyl, pyridazinyl, pyrimidinyl, thieno-[3,2-c]pyrazolyl, indolyl, 4,5,6,7-tetrahydrobenzo[b]thienyl, 4,7-dihydro-thieno[2,3-c]pyridinyl, or [1,2,4]triazolo[4,3-a]pyridinyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1b}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III), (XX), and (XXI), R^{3b} is pyrrol-2-yl, pyrrol-3-yl, thien-2-yl, thien-3-yl, thien-4-yl, thien-5-yl, isoxazol-3-yl, isoxazol-4-yl, isoxazol-5-yl, isothiazol-4-yl, isothiazol-5-yl, oxazol-2-yl, pyrrol-2-yl, pyrazol-4-yl, pyrazol-5-yl, thiazol-2-yl, thiazol-4-yl, thiazol-5-yl, 1,2,4-triazol-5-yl, 1,2,5-oxadiazol-3-yl, 1,3,4-oxadiazol-2-yl, 1,3,4-thiadiazol-2-yl, pyridin-2-yl, pyridin-3-yl, pyridazin-3-yl, pyrimidin-5-yl, thieno[3,2-c]pyrazol-3-yl, indol-2-yl, 4,5,6,7-tetrahydrobenzo[b]thien-2-yl, 4,7-dihydro-thieno[2,3-c]pyridin-2-yl, or [1,2,4]triazolo-[4,3-a]pyridin-3-yl, each optionally substituted with one, two, or three substituents, each of which is independently cyano, chloro, iodo, methyl, ethyl, isopropyl, tert-butyl, trifluoromethyl, 2-hydroxyethyl, cyclopropyl, phenyl, 3-chlorophenyl, furan-2-yl, pyrazol-1-yl, pyridin-2-yl, oxetan-3-yl, tetrahydropyran-4-yl, 2-chlorobenzoyl, methoxycarbonyl, ethoxycarbonyl, aminocarbonyl, isopropylaminocarbonyl, difluoromethoxy, amino, or methylthio.

In certain embodiments, in any one of Formulae (I) to (III), (XX), and (XXI), R^{3b} is 3-cyanopyrrol-2-yl, 3-cyano-4-methylpyrrol-2-yl, 2,4-diethoxycarbonylpyrrol-3-yl, 3-cyano-4,5-dimethylpyrrol-2-yl, 3-cyanothien-2-yl, 2-cyanothien-3-yl, 3-cyano-4-methylthien-2-yl, 3-(2-chlorobenzoyl)-5-ethylthien-2-yl, 2-methyl-3-methoxycarbonylthien-4-yl, 4-isopropyl-3-ethoxy-carbonylthien-2-yl, 3-ethoxycarbonyl-4-phenylthien-2-yl, 3,4-diethoxycarbonylthien-2-yl, 5-cyano-3-ethoxycarbonyl-2-(methylthio) thien-4-yl, 3-methyl-2-methoxycarbonylthien-5-yl, 3-cyano-4,5-dimethylthien-2-yl, 3-methoxycarbonyl-4,5-dimethylthien-2-yl, 5-iodo-2-methoxy-carbonylthien-3-yl, isoxazol-5-yl, 3-methylisoxazol-5-yl, 3-ethylisoxazol-5-yl, 5-ethylisoxazol-3-yl, 4-cyano-3-methylisoxazol-5-yl, 3,5-dimethylisoxazol-4-yl, 3-methylisothiazol-4-yl, 3-methylisothiazol-5-yl, 4-cyano-3-methylisothiazol-5-yl, 3-(isopropylaminocarbonyl)-4-amino-isothiazol-5-yl, 3-aminocarbonyl-4-aminoisothiazol-5-yl, oxazol-2-yl, 1-methylpyrazol-4-yl, 1-methylpyrazol-5-yl, 3-methylpyrazol-5-yl, 1-isopropylpyrazol-4-yl, 3-cyclopropylpyrazol-5-yl, 1-phenylpyrazol-4-yl, 3-phenylpyrazol-4-yl, 4-(3-chlorophenyl) pyrazol-5-yl, 3-(pyridin-2-yl)-pyrazol-4-yl, 1-(oxetan-3-yl) pyrazol-4-yl, 1-(tetrahydropyran-4-yl) pyrazol-4-yl, 3-(difluoro-methoxy) pyrazol-5-yl, 4-cyano-1-methylpyrazol-5-yl, 4-cyano-1-(2-hydroxyethyl) pyrazol-5-yl, 4-cyano-1-phenylpyrazol-5-yl, 3,5-dimethylpyrazol-4-yl, 1-(tert-butyl)-3-methylpyrazol-5-yl, 1-methyl-3-(trifluoromethyl) pyrazol-4-yl, 1-methyl-3-(trifluoromethyl) pyrazol-5-yl, 3-cyclo-propyl-1-methylpyrazol-5-yl, 3-(tert-butyl)-1-phenylpyrazol-5-yl, 4-ethoxycarbonyl-3-methyl-pyrazol-5-yl, 4-amino-1-methylpyrazol-5-yl, 4-cyano-1,3-dimethylpyrazol-5-yl, 4-cyano-3-methyl-1-phenylpyrazol-5-yl, thiazol-4-yl, 4-(trifluoromethyl) thiazol-2-yl, 4-(tert-butyl) thiazol-2-yl, 2-methylthiazol-5-yl, 4-ethoxycarbonylthiazol-5-yl, 1-methyl-1,2,4-triazol-5-yl, 4-methyl-1,2,5-oxadiazol-3-yl, 5-methyl-1,3,4-oxadiazol-2-yl, 5-(furan-2-yl)-1,3,4-oxadiazol-2-yl, 5-(tert-butyl)-1,3,4-thiadiazol-2-yl, pyridin-2-yl, 2-cyanopyridin-3-yl, 4-(pyrazol-1-yl)pyridin-2-yl, 6-chloro-3-cyanopyridin-2-yl, 2-cyano-5-methylpyridin-3-yl, pyridazin-3-yl, pyrimidin-5-yl, 3-methoxycarbonylindol-2-yl, 1-methyl-5-methoxycarbonylthieno[3,2-c]pyrazol-3-yl, 3-cyano-6-methyl-4,5,6,7-tetrahydrobenzo[b]thien-2-yl, 3-methoxycarbonyl-4,5,6,7-tetrahydrobenzo[b]-thien-2-yl, 3-ethoxycarbonyl-4-methyl-4,5,6,7-tetrahydrobenzo[b]thien-2-yl, 6-tert-butoxy-carboyl-3-ethoxycarbonyl-4,7-dihydrothieno[2,3-c]pyridin-2-yl, or 8-methyl[1,2,4]triazolo[4,3-a]pyridin-3-yl.

In certain embodiments, in any one of Formulae (I) to (III), (XX), and (XXI), R^{3b} is heterocyclyl, optionally substituted with one, two, or three substituents Q. In certain embodiments, in any one of Formulae (I) to (III), (XX), and (XXI), R^{3b} is monocyclic heterocyclyl, optionally substituted with one, two, or three substituents Q. In certain embodiments, in any one of Formulae (I) to (III), (XX), and (XXI), R^{3b} is 3-, 4-, 5-, 6-, or 7-membered heterocyclyl, each optionally substituted with one, two, or three substituents Q. In certain embodiments, in any one of Formulae (I) to (III), (XX), and (XXI), R^{3b} is 3-membered heterocyclyl, optionally substituted with one, two, or three substituents Q. In certain embodiments, in any one of Formulae (I) to (III), (XX), and (XXI), R^{3b} is 4-membered heterocyclyl, optionally substituted with one, two, or three substituents Q. In certain embodiments, in any one of Formulae (I) to (III), (XX), and (XXI), R^{3b} is 5-membered heterocyclyl, optionally substituted with one, two, or three substituents Q. In certain embodiments, in any one of Formulae (I) to (III), (XX), and (XXI), R^{3b} is 6-membered heterocyclyl, optionally substituted with one, two, or three substituents Q. In certain embodiments, in any one of Formulae (I) to (III), (XX), and (XXI), R^{3b} is 7-membered heterocyclyl, optionally substituted with one, two, or three substituents Q. In certain embodiments, in any one of Formulae (I) to (III), (XX), and (XXI), R^{3b} is bicyclic heterocyclyl, optionally substituted with one, two, or three substituents Q. In certain embodiments, in any one of Formulae (I) to (III), (XX), and (XXI), R^{3b} is bridged, fused, or spiro heterocyclyl, each optionally substituted with one, two, or three substituents Q. In certain embodiments, in any one of Formulae (I) to (III), (XX), and (XXI), R^{3b} is bridged heterocyclyl, optionally substituted with one, two, or three substituents Q. In certain embodiments, in any one of Formulae (I) to (III), (XX), and (XXI), R^{3b} is fused heterocyclyl, optionally substituted with one, two, or three substituents Q. In certain embodiments, in any one of Formulae (I) to (III), (XX), and (XXI), R^{3b} is spiro heterocyclyl, optionally substituted with one, two, or three substituents Q.

In certain embodiments, in any one of Formulae (I) to (III), (XX), and (XXI), R^{3b} is oxetanyl, azetidinyl, pyrrolidinyl, tetrahydrofuranyl, tetrahydropyranyl, piperidinyl, piperidinyl, thietanyl, tetrahydrothiopyranyl, or azepanyl, each optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1d}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein. In certain embodiments, in any one of Formulae (I) to (III), (XX), and (XXI), R^{3b} is oxetan-3-yl, azetidin-3-yl, pyrrolidin-3-yl, tetrahydrofuran-3-yl, tetrahydropyran-4-yl, piperidin-3-yl, piperidin-4-yl, thietan-3-yl, tetrahydrothiopyran-4-yl, or azepan-3-yl, each optionally substituted with one, two, or three substituents, each of which is independently oxo, methyl, propyl, or benzyl. In certain embodiments, in any one of Formulae (I) to (III), (XX), and (XXI), R^{3b} is oxetan-3-yl, 1-methylazetidin-3-yl, 1-methylpyrrolidin-3-yl, 1-benzylpyrrolidin-3-yl, 2-oxopyrrolidin-3-yl, tetrahydrofuran-3-yl, 2,3-dimethyltetrahydrofuran-3-yl, tetrahydro-pyran-4-yl, 4-methyltetrahydropyran-4-yl, 1-methylpiperidin-4-yl, 1-propylpiperidin-4-yl, 2-oxopiperidin-3-yl, 1-methyl-2-oxopiperidin-4-yl, 1,1-dioxidothietan-3-yl, 1,1-dioxidotetrahydro-thiopyran-4-yl, or 2-oxoazepan-3-yl.

In certain embodiments, in any one of Formulae (I) to (III), (XX), and (XXI), R^{3a} and R^{3b} together with the N atom to which they are attached form heterocyclyl, optionally substituted with one, two, or three substituents Q. In certain embodiments, in any one of Formulae (I) to (III), (XX), and (XXI), R^{3a} and R^{3b} together with the N atom to which they are attached form monocyclic heterocyclyl, optionally substituted with one, two, or three substituents Q. In certain embodiments, in any one of Formulae (I) to (III), (XX), and (XXI), R^{3a} and R^{3b} together with the N atom to which they are attached form 3-, 4-, 5-, 6-, or 7-membered heterocyclyl, each optionally substituted with one, two, or three substituents Q. In certain embodiments, in any one of Formulae (I) to (III), (XX), and (XXI), R^{3a} and R^{3b} together with the N atom to which they are attached form 3-membered heterocyclyl, optionally substituted with one, two, or three substituents Q. In certain embodiments, in any one of Formulae (I) to (III), (XX), and (XXI), R^{3a} and R^{3b} together with the N atom to which they are attached form 4-membered heterocyclyl, optionally substituted with one, two, or three substituents Q. In certain embodiments, in any one of Formulae (I) to (III), (XX), and (XXI), R^{3a} and R^{3b} together with the N atom to which they are attached form 5-membered heterocyclyl, optionally substituted with one, two, or three substituents Q. In certain embodiments, in any one of Formulae (I) to (III), (XX), and (XXI), R^{3a} and R^{3b} together with the N atom to which they are attached form 6-membered heterocyclyl, optionally substituted with one, two, or three substituents Q. In certain embodiments, in any one of Formulae (I) to (III), (XX), and (XXI), R^{3a} and R^{3b} together with the N atom to which they are attached form 7-membered heterocyclyl, optionally substituted with one, two, or three substituents Q. In certain embodiments, in any one of Formulae (I) to (III), (XX), and (XXI), R^{3a} and R^{3b} together with the N atom to which they are attached form piperazinyl, optionally substituted with one, two, or three substituents, each of which is independently (i) cyano, halo, nitro, or oxo; (ii) C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each optionally substituted with one, two, or three substituents Q; or (iii) —C(O)R^{1a}, —C(O)OR^{1a}, —C(O)NR^{1b}R^{1c}, —C(NR^{1a})NR^{1b}R^{1c}, —OR^{1a}, —NR^{1b}R^{1c}, —NR^{1a}C(O)R^{1d}, —NR^{1a}C(NR^{1d})NR^{1b}R^{1c}, —NR^{1a} S(O)₂R^{1b}, —SR^{1a}, —S(O)₂R^{1a}, or —S(O)₂NR^{1b}R^{1c}, wherein each R^{1a}, R^{1b}, R^{1c}, and R^{1d} is as defined herein.

In certain embodiments, in any one of Formulae (I) to (III), (XX), and (XXI), R^{3a} and R^{3b} together with the N atom to which they are attached form bicyclic heterocyclyl, optionally substituted with one, two, or three substituents Q. In certain embodiments, in any one of Formulae (I) to (III), (XX), and (XXI), R^{3a} and R^{3b} together with the N atom to which they are attached form bridged, fused, or spiro heterocyclyl, each optionally substituted with one, two, or three substituents Q. In certain embodiments, in any one of Formulae (I) to (III), (XX), and (XXI), R^{3a} and R^{3b} together with the N atom to which they are attached form bridged heterocyclyl, optionally substituted with one, two, or three substituents Q. In certain embodiments, in any one of Formulae (I) to (III), (XX), and (XXI), R^{3a} and R^{3b} together with the N atom to which they are attached form fused heterocyclyl, optionally substituted with one, two, or three substituents Q. In certain embodiments, in any one of Formulae (I) to (III), (XX), and (XXI), R^{3a} and R^{3b} together with the N atom to which they are attached form spiro heterocyclyl, optionally substituted with one, two, or three substituents Q.

In certain embodiments, in any one of Formulae (I) to (XXI), R¹ is hydrogen. In certain embodiments, in any one of Formulae (I) to (XXI), R¹ is deuterium. In certain embodiments, in any one of Formulae (I) to (XXI), R¹ is C₁₋₆ alkyl, optionally substituted with one, two, or three substituents Q. In certain embodiments, in any one of Formulae (I) to (XXI), R¹ is methyl.

In certain embodiments, in any one of Formulae (I) to (XXI), R² is hydrogen. In certain embodiments, in any one of Formulae (I) to (XXI), R² is C₁₋₆ alkyl, optionally substituted with one, two, or three substituents Q. In certain embodiments, in any one of Formulae (I) to (XXI), R² is methyl, optionally substituted with one, two, or three substituents Q. In certain embodiments, in any one of Formulae (I) to (XXI), R² is C₁₋₆ alkyl substituted with —C(O)R^{a} or —OP(O)(OR^{b})OR^{c}, wherein R^{a}, R^{b}, and R^{a} are each as defined herein. In certain embodiments, in any one of Formulae (I) to (XXI), R² is methyl substituted with —C(O)R^{a} or —OP(O)(OR^{b})OR″, wherein R^{a}, R^{b}, and R^{a} are each as defined herein. In certain embodiments, in any one of Formulae (I) to (XXI), R² is valyloxymethyl or di-tert-butoxyphosphoryloxymethyl. In certain embodiments, in any one of Formulae (I) to (XXI), R² is D-valyloxymethyl or L-valyloxymethyl.

In certain embodiments, in any one of Formulae (I) to (XIX), R³ is hydrogen. In certain embodiments, in any one of Formulae (I) to (XIX), R³ is C₁₋₆ alkyl, optionally substituted with one, two, or three substituents Q. In certain embodiments, in any one of Formulae (I) to (XIX), R³ is methyl, optionally substituted with one, two, or three substituents Q. In certain embodiments, in any one of Formulae (I) to (XIX), R³ is methyl.

In certain embodiments, in any one of Formulae (I) to (XIX), each R⁵ is independently halo. In certain embodiments, in any one of Formulae (I) to (XIX), each R⁵ is independently fluoro or chloro. In certain embodiments, in any one of Formulae (I) to (XIX), each R⁵ is fluoro.

In certain embodiments, in any one of Formulae (I) to (XXI), m is an integer of 0 or 1. In certain embodiments, in any one of Formulae (I) to (XXI), m is an integer of 0. In certain embodiments, in any one of Formulae (I) to (XXI), m is an integer of 1.

The groups, R¹, R², R³, R⁴, R⁵, R⁶, R⁷, R^{g}, R⁹, R^{3a}, R^{3b}, R^{4a}, R^{4b}, A, L³, L⁴, a, b, c, d, m, n, and p, in formulae described herein, including Formulae (I) to (XXI), are defined in the embodiments described herein. All combinations of the embodiments provided herein for such groups are within the scope of this disclosure.

In certain embodiments, R¹ is hydrogen. In certain embodiments, R¹ is deuterium. In certain embodiments, R¹ is C₁₋₆ alkyl, optionally substituted with one, two, or three substituents Q. In certain embodiments, R¹ is methyl, optionally substituted with one, two, or three substituents Q. In certain embodiments, R¹ is C₁₋₆ heteroalkyl, optionally substituted with one, two, or three substituents Q.

In certain embodiments, R² is hydrogen. In certain embodiments, R² is C₁₋₆ alkyl, optionally substituted with one, two, or three substituents Q. In certain embodiments, R² is C₁₋₆ alkyl substituted with —C(O)R^{a} or —OP(O)(OR^{b})OR^{c}, wherein R^{a}, R^{b}, and R^{a} are each as defined herein. In certain embodiments, R² is methyl substituted with —C(O)R^{a} or —OP(O)(OR^{b})OR^{c}, wherein R^{a}, R^{b}, and R^{a} are each as defined herein. In certain embodiments, R² is valyloxymethyl or di-tert-butoxyphosphoryloxymethyl. In certain embodiments, R² is D-valyloxymethyl or L-valyloxymethyl.

In certain embodiments, R³ is hydrogen. In certain embodiments, R³ is C₁₋₆ alkyl, optionally substituted with one, two, or three substituents Q. In certain embodiments, R³ is methyl, optionally substituted with one, two, or three substituents Q. In certain embodiments, R³ is C₁₋₆ heteroalkyl, optionally substituted with one, two, or three substituents Q. In certain embodiments, R³ is

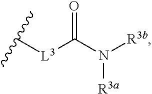

wherein R^{3a}, R^{3b}, and L³ are each as defined herein.

In certain embodiments, R⁴ is

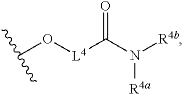

wherein R^{4a}, R^{4b}, and L⁴ are each as defined herein. In certain embodiments, R⁴ is

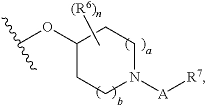

wherein R⁶, R⁷, A, a, b, and c are each as defined herein.

In certain embodiments, R⁵ is deuterium. In certain embodiments, R⁵ is cyano. In certain embodiments, R⁵ is halo. In certain embodiments, R⁵ is fluoro. In certain embodiments, R⁵ is chloro. In certain embodiments, R⁵ is nitro. In certain embodiments, R⁵ is C₁₋₆ alkyl, optionally substituted with one, two, or three substituents Q. In certain embodiments, R⁵ is methyl, ethyl, or isopropyl. In certain embodiments, R⁵ is C₁₋₆ heteroalkyl, optionally substituted with one, two, or three substituents Q. In certain embodiments, R⁵ is C₂₋₆ alkenyl, optionally substituted with one, two, or three substituents Q. In certain embodiments, R⁵ is C₂₋₆ alkynyl, optionally substituted with one, two, or three substituents Q. In certain embodiments, R⁵ is C₃₋₁₀ cycloalkyl, optionally substituted with one, two, or three substituents Q. In certain embodiments, R⁵ is C₆₋₁₄ aryl, optionally substituted with one, two, or three substituents Q. In certain embodiments, R⁵ is C₇₋₁₅ aralkyl, optionally substituted with one, two, or three substituents Q. In certain embodiments, R⁵ is heteroaryl, optionally substituted with one, two, or three substituents Q. In certain embodiments, R⁵ is heterocyclyl, optionally substituted with one, two, or three substituents Q.

In certain embodiments, R⁵ is —C(O)R^{1a}, wherein R^{1a} is as defined herein. In certain embodiments, R⁵ is —C(O)OR^{1a}, wherein R^{1a} is as defined herein. In certain embodiments, R⁵ is —C(O)NR^{1b}R^{1c}, wherein R^{1b} and R^{1a} are each as defined herein. In certain embodiments, R⁵ is —C(O)SR^{1a}, wherein R^{1a} is as defined herein. In certain embodiments, R⁵ is —C(NR^{1a})NR^{1b}R^{1c}, wherein R^{1a}, R^{1b}, and R^{1c} are each as defined herein. In certain embodiments, R⁵ is —C(S)R^{1a}, wherein R^{1a} is as defined herein. In certain embodiments, R⁵ is —C(S)OR^{1a}, wherein R^{1a} is as defined herein. In certain embodiments, R⁵ is —C(S)NR^{1b}R^{1c}, wherein R^{1b} and R^{1a} are each as defined herein. In certain embodiments, R⁵ is —OR^{1a}, wherein R^{1a} is as defined herein. In certain embodiments, R⁵ is methoxy. In certain embodiments, R⁵ is —OC(O)R^{1a}, wherein R^{1a} is as defined herein. In certain embodiments, R⁵ is —OC(O)OR^{1a}, wherein R^{1a} is as defined herein. In certain embodiments, R⁵ is —OC(O)NR^{1b}R^{1c}, wherein R^{1b} and R^{1a} are each as defined herein. In certain embodiments, R⁵ is —OC(O)SR^{1a}, wherein R^{1a} is as defined herein. In certain embodiments, R⁵ is —OC(NR^{1a})NR^{1b}R^{1c}, wherein R^{1a}, R^{1b}, and R^{1a} are each as defined herein. In certain embodiments, R⁵ is —OC(S)R^{1a}, wherein R^{1a} is as defined herein. In certain embodiments, R⁵ is —OC(S)OR^{1a}, wherein R^{1a} is as defined herein. In certain embodiments, R⁵ is —OC(S)NR^{1b}R^{1c}, wherein R^{1b} and R^{1a} are each as defined herein. In certain embodiments, R⁵ is —OS(O)R^{1a}, wherein R^{1a} is as defined herein. In certain embodiments, R⁵ is —OS(O)₂R^{1a}, wherein R^{1a} is as defined herein. In certain embodiments, R⁵ is —OS(O)NR^{1b}R^{1c}, wherein R^{1b} and R^{1c} are each as defined herein. In certain embodiments, R⁵ is —OS(O)₂NR^{1b}R^{1c}, wherein R^{1b} and R^{1c} are each as defined herein. In certain embodiments, R⁵ is —NR^{1b}R^{1c}, wherein R^{1b} and R^{1c} are each as defined herein. In certain embodiments, R⁵ is —NR^{1a}C(O)R^{1d}, wherein R^{1a} and R^{1d} are each as defined herein. In certain embodiments, R⁵ is —NR^{1a}C(O)OR^{1d}, wherein R^{1a} and R^{1d} are each as defined herein. In certain embodiments, R⁵ is —NR^{1a}C(O)NR^{1b}R^{1c}, wherein R^{1a}, R^{1b}, and R^{1a} are each as defined herein. In certain embodiments, R⁵ is —NR^{1a}C(O)SR^{1d}, wherein R^{1a} and R^{1d} are each as defined herein. In certain embodiments, R⁵ is —NR^{1a}C(NR^{1d})NR^{1b}R^{1c}, wherein R^{1a}, R^{1b}, R^{1c}, and R^{1d} are each as defined herein. In certain embodiments, R⁵ is —NR^{1a}C(S)R^{1d}, wherein R^{1a} and R^{1d} are each as defined herein. In certain embodiments, R⁵ is —NR^{1a}C(S)OR^{1d}, wherein R^{1a} and R^{1d} are each as defined herein. In certain embodiments, R⁵ is —NR^{1a}C(S)NR^{1b}R^{1c}, wherein R^{1a}, R^{1b}, and R^{1a} are each as defined herein. In certain embodiments, R⁵ is —NR^{1a} S(O)R^{1d}, wherein R^{1a} and R^{1d} are each as defined herein. In certain embodiments, R⁵ is —NR^{1a} S(O)₂R^{1d}, wherein R^{1a} and R^{1d} are each as defined herein. In certain embodiments, R⁵ is —NR^{1a} S(O)NR^{1b}R^{1c}, wherein R^{1a}, R^{1b}, and R^{1a} are each as defined herein. In certain embodiments, R⁵ is —NR^{1a} S(O)₂NR^{1b}R^{1c}, wherein R^{1a}, R^{1b}, and R^{1a} are each as defined herein. In certain embodiments, R⁵ is —SR^{1a}, wherein R^{1a} is as defined herein. In certain embodiments, R⁵ is —S(O)R^{1a}, wherein R^{1a} is as defined herein. In certain embodiments, R⁵ is —S(O)₂R^{1a}, wherein R^{1a} is as defined herein. In certain embodiments, R⁵ is —S(O)NR^{1b}R^{1c}, wherein R^{1b} and R^{1c} are each as defined herein. In certain embodiments, R⁵ is —S(O)₂NR^{1b}R^{1c}, wherein R^{1b} and R^{1a} are each as defined herein.

In certain embodiments, R⁶ is deuterium. In certain embodiments, R⁶ is cyano. In certain embodiments, R⁶ is halo. In certain embodiments, R⁶ is fluoro. In certain embodiments, R⁶ is chloro. In certain embodiments, R⁶ is nitro. In certain embodiments, R⁶ is C₁₋₆ alkyl, optionally substituted with one, two, or three substituents Q. In certain embodiments, R⁶ is methyl, ethyl, or isopropyl. In certain embodiments, R⁶ is C₁₋₆ heteroalkyl, optionally substituted with one, two, or three substituents Q. In certain embodiments, R⁶ is C₂₋₆ alkenyl, optionally substituted with one, two, or three substituents Q. In certain embodiments, R⁶ is C₂₋₆ alkynyl, optionally substituted with one, two, or three substituents Q. In certain embodiments, R⁶ is C₃₋₁₀ cycloalkyl, optionally substituted with one, two, or three substituents Q. In certain embodiments, R⁶ is C₆₋₁₄ aryl, optionally substituted with one, two, or three substituents Q. In certain embodiments, R⁶ is C₇₋₁₅ aralkyl, optionally substituted with one, two, or three substituents Q. In certain embodiments, R⁶ is heteroaryl, optionally substituted with one, two, or three substituents Q. In certain embodiments, R⁶ is heterocyclyl, optionally substituted with one, two, or three substituents Q.

In certain embodiments, R⁶ is —C(O)R^{1a}, wherein R^{1a} is as defined herein. In certain embodiments, R⁶ is —C(O)OR^{1a}, wherein R^{1a} is as defined herein. In certain embodiments, R⁶ is —C(O)NR^{1b}R^{1c}, wherein R^{1b} and R^{1a} are each as defined herein. In certain embodiments, R⁶ is —C(O)SR^{1a}, wherein R^{1a} is as defined herein. In certain embodiments, R⁶ is —C(NR^{1a})NR^{1b}R^{1c}, wherein R^{1a}, R^{1b}, and R^{c} are each as defined herein. In certain embodiments, R⁶ is —C(S)R^{1a}, wherein R^{1a} is as defined herein. In certain embodiments, R⁶ is —C(S)OR^{1a}, wherein R^{1a} is as defined herein. In certain embodiments, R⁶ is —C(S)NR^{1b}R^{1c}, wherein R^{1b} and R^{1a} are each as defined herein. In certain embodiments, R⁶ is —OR^{1a}, wherein R^{1a} is as defined herein. In certain embodiments, R⁶ is methoxy. In certain embodiments, R⁶ is —OC(O)R^{1a}, wherein R^{1a} is as defined herein. In certain embodiments, R⁶ is —OC(O)OR^{1a}, wherein R^{1a} is as defined herein. In certain embodiments, R⁶ is —OC(O)NR^{1b}R^{1c}, wherein R^{1b} and R^{1a} are each as defined herein. In certain embodiments, R⁶ is —OC(O)SR^{1a}, wherein R^{1a} is as defined herein. In certain embodiments, R⁶ is —OC(NR^{1a})NR^{1b}R^{1c}, wherein R^{1a}, R^{1b}, and R^{1a} are each as defined herein. In certain embodiments, R⁶ is —OC(S)R^{1a}, wherein R^{1a} is as defined herein. In certain embodiments, R⁶ is —OC(S)OR^{1a}, wherein R^{1a} is as defined herein. In certain embodiments, R⁶ is —OC(S)NR^{1b}R^{1c}, wherein R^{1b} and R^{1a} are each as defined herein. In certain embodiments, R⁶ is —OS(O)R^{1a}, wherein R^{1a} is as defined herein. In certain embodiments, R⁶ is —OS(O)₂R^{1a} wherein R^{1a} is as defined herein. In certain embodiments, R⁶ is —OS(O)NR^{1b}R^{1c}, wherein R^{1b} and R^{1a} are each as defined herein. In certain embodiments, R⁶ is —OS(O)₂NR^{1b}R^{1c}, wherein R^{1b} and R^{1a} are each as defined herein. In certain embodiments, R⁶ is —NR^{1b}R^{1c}, wherein R^{1b} and R^{1c} are each as defined herein. In certain embodiments, R⁶ is —NR^{1a}C(O)R^{1d}, wherein R^{a} and R^{1d} are each as defined herein. In certain embodiments, R⁶ is —NR^{1a}C(O)OR^{1d}, wherein R^{1a} and R^{1d} are each as defined herein. In certain embodiments, R⁶ is —NR^{1a}C(O)NR^{1b}R^{1c}, wherein R^{1a}, R^{1b}, and R^{1a} are each as defined herein. In certain embodiments, R⁶ is —NR^{1a}C(O)SR^{1d}, wherein R^{1a} and R^{1d} are each as defined herein. In certain embodiments, R⁶ is —NR^{1a}C(NR^{1d})NR^{1b}R^{1c}, wherein R^{1a}, R^{1b}, R^{1c}, and R^{1d} are each as defined herein. In certain embodiments, R⁶ is —NR^{1a}C(S)R^{1b}, wherein R^{1a} and R^{1d} are each as defined herein. In certain embodiments, R⁶ is —NR^{1a}C(S)OR^{1d}, wherein R^{1a} and R^{1d} are each as defined herein. In certain embodiments, R⁶ is —NR^{1a}C(S)NR^{1b}R^{1c}, wherein R^{1a}, R^{1b}, and R^{1a} are each as defined herein. In certain embodiments, R⁶ is —NR^{1a} S(O)R^{1d}, wherein R^{1a} and R^{1d} are each as defined herein. In certain embodiments, R⁶ is —NR^{1a} S(O)₂R^{1d}, wherein R^{1a} and R^{1d} are each as defined herein. In certain embodiments, R⁶ is —NR^{1a} S(O)NR^{1b}R^{1c}, wherein R^{1a}, R^{1b}, and R^{1c} are each as defined herein. In certain embodiments, R⁶ is —NR^{1a} S(O)₂NR^{1b}R^{1c}, wherein R^{1a}, R^{1b}, and R^{1a} are each as defined herein. In certain embodiments, R⁶ is —SR^{1a}, wherein R^{1a} is as defined herein. In certain embodiments, R⁶ is —S(O)R^{1a}, wherein R^{1a} is as defined herein. In certain embodiments, R⁶ is —S(O)₂R^{1a}, wherein R^{1a} is as defined herein. In certain embodiments, R⁶ is —S(O)NR^{1b}R^{1c}, wherein R^{1b} and R^{1c} are each as defined herein. In certain embodiments, R⁶ is —S(O)₂NR^{1b}R^{1c}, wherein R^{1b} and R^{1a} are each as defined herein.

In certain embodiments, R⁷ is hydrogen. In certain embodiments, R⁷ is deuterium. In certain embodiments, R⁷ is C₁₋₆ alkyl, optionally substituted with one, two, or three substituents Q. In certain embodiments, R⁷ is C₁₋₆ heteroalkyl, optionally substituted with one, two, or three substituents Q. In certain embodiments, R⁷ is C₂₋₆ alkenyl, optionally substituted with one, two, or three substituents Q. In certain embodiments, R⁷ is C₂₋₆ alkynyl, optionally substituted with one, two, or three substituents Q. In certain embodiments, R⁷ is C₃₋₁₀ cycloalkyl, optionally substituted with one, two, or three substituents Q. In certain embodiments, R⁷ is C₆₋₁₄ aryl, optionally substituted with one, two, or three substituents Q. In certain embodiments, R⁷ is C₇₋₁₅ aralkyl, optionally substituted with one, two, or three substituents Q. In certain embodiments, R⁷ is heteroaryl, optionally substituted with one, two, or three substituents Q. In certain embodiments, R⁷ is heterocyclyl, optionally substituted with one, two, or three substituents Q.

In certain embodiments, R⁷ is —OR^{1a}, wherein R^{1a} is as defined herein. In certain embodiments, R⁷ is methoxy. In certain embodiments, R⁷ is —OC(O)R^{1a}, wherein R^{1a} is as defined herein. In certain embodiments, R⁷ is —OC(O)OR^{1a}, wherein R^{1a} is as defined herein. In certain embodiments, R⁷ is —OC(O)NR^{1b}R^{1c}, wherein R^{1b} and R^{1a} are each as defined herein. In certain embodiments, R⁷ is —OC(O)SR^{1a}, wherein R^{1a} is as defined herein. In certain embodiments, R⁷ is —OC(NR^{1a})NR^{1b}R^{1c}, wherein R^{1a}, R^{1b}, and R^{1a} are each as defined herein. In certain embodiments, R⁷ is —OC(S)R^{1a}, wherein R^{1a} is as defined herein. In certain embodiments, R⁷ is —OC(S)OR^{1a}, wherein R^{1a} is as defined herein. In certain embodiments, R⁷ is —OC(S)NR^{1b}R^{1c}, wherein R^{1b} and R^{1c} are each as defined herein. In certain embodiments, R⁷ is —OS(O)R^{1a}, wherein R^{1a} is as defined herein. In certain embodiments, R⁷ is —OS(O)₂R^{1a}, wherein R^{1a} is as defined herein. In certain embodiments, R⁷ is —OS(O)NR^{1b}R^{1c}, wherein R^{1b} and R^{1c} are each as defined herein. In certain embodiments, R⁷ is —OS(O)₂NR^{1b}R^{1c}, wherein R^{1b} and R^{1a} are each as defined herein. In certain embodiments, R⁷ is —NR^{1b}R^{1c}, wherein R^{1b} and R^{1c} are each as defined herein. In certain embodiments, R⁷ is —NR^{1a}C(O)R^{1d}, wherein R^{1a} and R^{1d} are each as defined herein. In certain embodiments, R⁷ is —NR^{1a}C(O)OR^{1d}, wherein R^{1a} and R^{1d} are each as defined herein. In certain embodiments, R⁷ is —NR^{1a}C(O)NR^{1b}R^{1c}, wherein R^{1a}, R^{1b}, and R^{1a} are each as defined herein. In certain embodiments, R⁷ is —NR^{1a}C(O)SR^{1d}, wherein R^{1a} and R^{1d} are each as defined herein. In certain embodiments, R⁷ is —NR^{1a}C(NR^{1d})NR^{1b}R^{1c}, wherein R^{a}, R^{1b}, R^{1c}, and R^{1d} are each as defined herein. In certain embodiments, R⁷ is —NR^{1a}C(S)R^{1d}, wherein R^{1a} and R^{1d} are each as defined herein. In certain embodiments, R⁷ is —NR^{1a}C(S)OR^{1d}, wherein R^{1a} and R^{1d} are each as defined herein. In certain embodiments, R⁷ is —NR^{1a}C(S)NR^{1b}R^{1c}, wherein R^{1a}, R^{1b}, and R^{1a} are each as defined herein. In certain embodiments, R⁷ is —NR^{1a} S(O)R^{1b}, wherein R^{1a} and R^{1d} are each as defined herein. In certain embodiments, R⁷ is —NR^{1a} S(O)₂R^{1d}, wherein R^{1a} and R^{1d} are each as defined herein. In certain embodiments, R⁷ is —NR^{1a} S(O)NR^{1b}R^{1c}, wherein R^{1a}, R^{1b}, and R^{1c} are each as defined herein. In certain embodiments, R⁷ is —NR^{1a} S(O)₂NR^{1b}R^{1c}, wherein R^{1a}, R^{1b}, and R^{1c} are each as defined herein.

In certain embodiments, R^{g} is deuterium. In certain embodiments, R^{g} is cyano. In certain embodiments, R^{g} is halo. In certain embodiments, R^{g} is fluoro. In certain embodiments, R^{g} is chloro. In certain embodiments, R^{g} is nitro. In certain embodiments, R^{g} is C₁₋₆ alkyl, optionally substituted with one, two, or three substituents Q. In certain embodiments, R^{g} is methyl, ethyl, or isopropyl. In certain embodiments, R^{g} is C₁₋₆ heteroalkyl, optionally substituted with one, two, or three substituents Q. In certain embodiments, R^{g} is C₂₋₆ alkenyl, optionally substituted with one, two, or three substituents Q. In certain embodiments, R^{g} is C₂₋₆ alkynyl, optionally substituted with one, two, or three substituents Q. In certain embodiments, R^{g} is C₃₋₁₀ cycloalkyl, optionally substituted with one, two, or three substituents Q. In certain embodiments, R^{g} is C₆₋₁₄ aryl, optionally substituted with one, two, or three substituents Q. In certain embodiments, R^{g} is C₇₋₁₅ aralkyl, optionally substituted with one, two, or three substituents Q. In certain embodiments, R^{g} is heteroaryl, optionally substituted with one, two, or three substituents Q. In certain embodiments, R^{g} is heterocyclyl, optionally substituted with one, two, or three substituents Q.

In certain embodiments, R^{g} is —C(O)R^{1a}, wherein R^{1a} is as defined herein. In certain embodiments, R^{g} is —C(O)OR^{1a}, wherein R^{1a} is as defined herein. In certain embodiments, R& is —C(O)NR^{1b}R^{1c}, wherein R^{1b} and R^{1a} are each as defined herein. In certain embodiments, R^{g} is —C(O)SR^{1a}, wherein R^{1a} is as defined herein. In certain embodiments, R^{g} is —C(NR^{1a})NR^{1b}R^{1c}, wherein R^{1a}, R^{1b}, and R^{1a} are each as defined herein. In certain embodiments, R^{g} is —C(S)R^{1a}, wherein R^{1a} is as defined herein. In certain embodiments, R^{g} is —C(S)OR^{1a}, wherein R^{1a} is as defined herein. In certain embodiments, R^{g} is —C(S)NR^{1b}R^{1c}, wherein R^{1b} and R^{1a} are each as defined herein. In certain embodiments, R^{g} is —OR^{1a}, wherein R^{1a} is as defined herein. In certain embodiments, R^{g} is methoxy. In certain embodiments, R^{g} is —OC(O)R^{1a}, wherein R^{1a} is as defined herein. In certain embodiments, R^{g} is —OC(O)OR^{1a}, wherein R^{1a} is as defined herein. In certain embodiments, R^{g} is —OC(O)NR^{1b}R^{1c}, wherein R^{1b} and R^{1a} are each as defined herein. In certain embodiments, R^{g} is —OC(O)SR^{1a}, wherein R^{1a} is as defined herein. In certain embodiments, R^{g} is —OC(NR^{1a})NR^{1b}R^{1c}, wherein R^{1a}, R^{1b}, and R^{1a} are each as defined herein. In certain embodiments, R^{g} is —OC(S)R^{1a}, wherein R^{1a} is as defined herein. In certain embodiments, R^{g} is —OC(S)OR^{1a}, wherein R^{1a} is as defined herein. In certain embodiments, R^{g} is —OC(S)NR^{1b}R^{1c}, wherein R^{1b} and R^{1a} are each as defined herein. In certain embodiments, R^{g} is —OS(O)R^{1a}, wherein R^{1a} is as defined herein. In certain embodiments, R^{g} is —OS(O)₂R^{1a} wherein R^{1a} is as defined herein. In certain embodiments, R^{g} is —OS(O)NR^{1b}R^{1c}, wherein R^{1b} and R^{1c} are each as defined herein. In certain embodiments, R^{g} is —OS(O)₂NR^{1b}R^{1c}, wherein R^{1b} and R^{1a} are each as defined herein. In certain embodiments, R⁸ is —NR^{1b}R^{1c}, wherein R^{1b} and R^{1c} are each as defined herein. In certain embodiments, R⁸ is —NR^{1a}C(O)R^{1d}, wherein R^{1a} and R^{1d} are each as defined herein. In certain embodiments, R⁸ is —NR^{1a}C(O)OR^{1d}, wherein R^{1a} and R^{1d} are each as defined herein. In certain embodiments, R⁸ is —NR^{1a}C(O)NR^{1b}R^{1c}, wherein R^{1a}, R^{1b}, and R^{1a} are each as defined herein. In certain embodiments, R⁸ is —NR^{1a}C(O)SR^{1d}, wherein R^{1a} and R^{1d} are each as defined herein. In certain embodiments, R⁸ is —NR^{1a}C(NR^{1d})NR^{1b}R^{1c}, wherein R^{1a}, R^{1b}, R^{1c}, and R^{1d} are each as defined herein. In certain embodiments, R⁸ is —NR^{1a}C(S)R^{1b}, wherein R^{1a} and R^{1d} are each as defined herein. In certain embodiments, R⁸ is —NR^{1a}C(S)OR^{1d}, wherein R^{1a} and R^{1d} are each as defined herein. In certain embodiments, R⁸ is —NR^{1a}C(S)NR^{1b}R^{1c}, wherein R^{1a}, R^{1b}, and R^{1a} are each as defined herein. In certain embodiments, R⁸ is —NR^{1a} S(O)R^{1d}, wherein R^{1a} and R^{d} are each as defined herein. In certain embodiments, R⁸ is —NR^{1a} S(O)₂R^{1d}, wherein R^{1a} and R^{1d} are each as defined herein. In certain embodiments, R⁸ is —NR^{1a} S(O)NR^{1b}R^{1c}, wherein R^{1a}, R^{1b}, and R^{1a} are each as defined herein. In certain embodiments, R⁸ is —NR^{1a} S(O)₂NR^{1b}R^{1c}, wherein R^{1a}, R^{1b}, and R^{1a} are each as defined herein. In certain embodiments, R^{g} is —SR^{1a}, wherein R^{1a} is as defined herein. In certain embodiments, R^{g} is —S(O)R^{1a}, wherein R^{1a} is as defined herein. In certain embodiments, R^{g} is —S(O)₂R^{1a}, wherein R^{1a} is as defined herein. In certain embodiments, R^{g} is —S(O)NR^{1b}R^{1c}, wherein R^{1b} and R^{1c} are each as defined herein. In certain embodiments, R^{g} is —S(O)₂NR^{1b}R^{1c}, wherein R^{1b} and R^{1a} are each as defined herein.

In certain embodiments, R⁹ is hydrogen. In certain embodiments, R⁹ is C₁₋₆ alkyl, optionally substituted with one, two, or three substituents Q. In certain embodiments, R⁹ is C₁₋₆ heteroalkyl, optionally substituted with one, two, or three substituents Q. In certain embodiments, R⁹ is C₂₋₆ alkenyl, optionally substituted with one, two, or three substituents Q. In certain embodiments, R⁹ is C₂₋₆ alkynyl, optionally substituted with one, two, or three substituents Q. In certain embodiments, R⁹ is C₃₋₁₀ cycloalkyl, optionally substituted with one, two, or three substituents Q. In certain embodiments, R⁹ is C₆₋₁₄ aryl, optionally substituted with one, two, or three substituents Q. In certain embodiments, R⁹ is C₇₋₁₅ aralkyl, optionally substituted with one, two, or three substituents Q. In certain embodiments, R⁹ is heteroaryl, optionally substituted with one, two, or three substituents Q. In certain embodiments, R⁹ is heterocyclyl, optionally substituted with one, two, or three substituents Q.

In certain embodiments, R⁹ is —C(O)R^{1a}, wherein R^{1a} is as defined herein. In certain embodiments, R⁹ is —C(O)OR^{1a}, wherein R^{1a} is as defined herein. In certain embodiments, R⁹ is —C(O)NR^{1b}R^{1c}, wherein R^{1b} and R^{1c} are each as defined herein. In certain embodiments, R⁹ is —C(O)SR^{1a}, wherein R^{1a} is as defined herein. In certain embodiments, R⁹ is —C(NR^{1a})NR^{1b}R^{1c}, wherein R^{1a}, R^{1b}, and R^{1a} are each as defined herein. In certain embodiments, R⁹ is —C(S)R^{1a}, wherein R^{1a} is as defined herein. In certain embodiments, R⁹ is —C(S)OR^{1a}, wherein R^{1a} is as defined herein. In certain embodiments, R⁹ is —C(S)NR^{1b}R^{1c}, wherein R^{1b} and R^{1a} are each as defined herein. In certain embodiments, R⁹ is —S(O)R^{1a}, wherein R^{1a} is as defined herein. In certain embodiments, R⁹ is —S(O)₂R^{1a}, wherein R^{1a} is as defined herein. In certain embodiments, R⁹ is —S(O)NR^{1b}R^{1c}, wherein R^{1b} and R^{1c} are each as defined herein. In certain embodiments, R⁹ is —S(O)₂NR^{1b}R^{1c}, wherein R^{1b} and R^{1c} are each as defined herein.

In certain embodiments, R^{3a} is hydrogen. In certain embodiments, R^{3a} is C₁₋₆ alkyl, optionally substituted with one, two, or three substituents Q. In certain embodiments, R^{3a} is C₁₋₆ heteroalkyl, optionally substituted with one, two, or three substituents Q. In certain embodiments, R^{3a} is C₂₋₆ alkenyl, optionally substituted with one, two, or three substituents Q. In certain embodiments, R^{3a} is C₂₋₆ alkynyl, optionally substituted with one, two, or three substituents Q. In certain embodiments, R^{3a} is C₃₋₁₀ cycloalkyl, optionally substituted with one, two, or three substituents Q. In certain embodiments, R^{3a} is C₆₋₁₄ aryl, optionally substituted with one, two, or three substituents Q. In certain embodiments, R^{3a} is C₇₋₁₅ aralkyl, optionally substituted with one, two, or three substituents Q. In certain embodiments, R^{3a} is heteroaryl, optionally substituted with one, two, or three substituents Q. In certain embodiments, R^{3a} is heterocyclyl, optionally substituted with one, two, or three substituents Q.

In certain embodiments, R^{3a} is —C(O)R^{1a}, wherein R^{1a} is as defined herein. In certain embodiments, R^{3a} is —C(O)OR^{1a}, wherein R^{1a} is as defined herein. In certain embodiments, R^{3a} is —C(O)NR^{1b}R^{1c}, wherein R^{1b} and R^{1c} are each as defined herein. In certain embodiments, R^{3a} is —C(O)SR^{1a}, wherein R^{1a} is as defined herein. In certain embodiments, R^{3a} is —C(NR^{1a})NR^{1b}R^{1c}, wherein R^{1a}, R^{1b}, and R^{1a} are each as defined herein. In certain embodiments, R^{3a} is —C(S)R^{1a}, wherein R^{1a} is as defined herein. In certain embodiments, R^{3a} is —C(S)OR^{1a}, wherein R^{1a} is as defined herein. In certain embodiments, R^{3a} is —C(S)NR^{1b}R^{1c}, wherein R^{1b} and R^{1a} are each as defined herein. In certain embodiments, R^{3a} is —S(O)R^{1a}, wherein R^{1a} is as defined herein. In certain embodiments, R^{3a} is —S(O)₂R^{1a}, wherein R^{1a} is as defined herein. In certain embodiments, R^{3a} is —S(O)NR^{1b}R^{1c}, wherein R^{1b} and R^{1a} are each as defined herein. In certain embodiments, R^{3a} is —S(O)₂NR^{1b}R^{1c}, wherein R^{1b} and R^{1c} are each as defined herein.

In certain embodiments, R^{3b} is hydrogen. In certain embodiments, R^{3b} is C₁₋₆ alkyl, optionally substituted with one, two, or three substituents Q. In certain embodiments, R^{3b} is C₁₋₆ heteroalkyl, optionally substituted with one, two, or three substituents Q. In certain embodiments, R^{3b} is C₂₋₆ alkenyl, optionally substituted with one, two, or three substituents Q. In certain embodiments, R^{3b} is C₂₋₆ alkynyl, optionally substituted with one, two, or three substituents Q. In certain embodiments, R^{3b} is C₃₋₁₀ cycloalkyl, optionally substituted with one, two, or three substituents Q. In certain embodiments, R^{3b} is monocyclic C₃₋₁₀ cycloalkyl, optionally substituted with one, two, or three substituents Q. In certain embodiments, R^{3b} is bicyclic C₄₋₁₀ cycloalkyl, optionally substituted with one, two, or three substituents Q. In certain embodiments, R^{3b} is bridged, fused, or spiro C₄₋₁₀ cycloalkyl, each optionally substituted with one, two, or three substituents Q. In certain embodiments, R^{3b} is bridged C₄₋₁₀ cycloalkyl, optionally substituted with one, two, or three substituents Q. In certain embodiments, R^{3b} is fused C₄₋₁₀ cycloalkyl, optionally substituted with one, two, or three substituents Q. In certain embodiments, R^{3b} is spiro C₄₋₁₀ cycloalkyl, optionally substituted with one, two, or three substituents Q. In certain embodiments, R^{3b} is C₆₋₁₄ aryl, optionally substituted with one, two, or three substituents Q. In certain embodiments, R^{3b} is phenyl, optionally substituted with one, two, or three substituents Q. In certain embodiments, R^{3b} is bicyclic C₈₋₁₄ aryl, optionally substituted with one, two, or three substituents Q. In certain embodiments, R^{3b} is C₇₋₁₅ aralkyl, optionally substituted with one, two, or three substituents Q.

In certain embodiments, R^{3b} is heteroaryl, optionally substituted with one, two, or three substituents Q. In certain embodiments, R^{3b} is monocyclic heteroaryl, optionally substituted with one, two, or three substituents Q. In certain embodiments, R^{3b} is 5- or 6-membered heteroaryl, each optionally substituted with one, two, or three substituents Q. In certain embodiments, R^{3b} is 5-membered heteroaryl, optionally substituted with one, two, or three substituents Q. In certain embodiments, R^{3b} is 6-membered heteroaryl, optionally substituted with one, two, or three substituents Q. In certain embodiments, R^{3b} is bicyclic heteroaryl, optionally substituted with one, two, or three substituents Q. In certain embodiments, R^{3b} is 5,5-, 5,6-, or 6,6-fused heteroaryl, each optionally substituted with one, two, or three substituents Q. In certain embodiments, R^{3b} is 5,5-fused heteroaryl, optionally substituted with one, two, or three substituents Q. In certain embodiments, R^{3b} is 5,6-fused heteroaryl, optionally substituted with one, two, or three substituents Q. In certain embodiments, R^{3b} is 6,6-fused heteroaryl, optionally substituted with one, two, or three substituents Q.

In certain embodiments, R^{3b} is heterocyclyl, optionally substituted with one, two, or three substituents Q. In certain embodiments, R^{3b} is monocyclic heterocyclyl, optionally substituted with one, two, or three substituents Q. In certain embodiments, R^{3b} is 3-, 4-, 5-, 6-, or 7-membered heterocyclyl, each optionally substituted with one, two, or three substituents Q. In certain embodiments, R^{3b} is 3-membered heterocyclyl, optionally substituted with one, two, or three substituents Q. In certain embodiments, R^{3b} is 4-membered heterocyclyl, optionally substituted with one, two, or three substituents Q. In certain embodiments, R^{3b} is 5-membered heterocyclyl, optionally substituted with one, two, or three substituents Q. In certain embodiments, R^{3b} is 6-membered heterocyclyl, optionally substituted with one, two, or three substituents Q. In certain embodiments, R^{3b} is 7-membered heterocyclyl, optionally substituted with one, two, or three substituents Q. In certain embodiments, R^{3b} is bicyclic heterocyclyl, optionally substituted with one, two, or three substituents Q. In certain embodiments, R^{3b} is bridged, fused, or spiro heterocyclyl, each optionally substituted with one, two, or three substituents Q. In certain embodiments, R^{3b} is bridged heterocyclyl, optionally substituted with one, two, or three substituents Q. In certain embodiments, R^{3b} is fused heterocyclyl, optionally substituted with one, two, or three substituents Q. In certain embodiments, R^{3b} is spiro heterocyclyl, optionally substituted with one, two, or three substituents Q.

In certain embodiments, R^{3b} is —C(O)R^{1a}, wherein R^{1a} is as defined herein. In certain embodiments, R^{3b} is —C(O)OR^{1a}, wherein R^{1a} is as defined herein. In certain embodiments, R^{3b} is —C(O)NR^{1b}R^{1c}, wherein R^{1b} and R^{1c} are each as defined herein. In certain embodiments, R^{3b} is —C(O)SR^{1a}, wherein R^{1a} is as defined herein. In certain embodiments, R^{3b} is —C(NR^{1a})NR^{1b}R^{1c}, wherein R^{1a}, R^{1b}, and R^{1a} are each as defined herein. In certain embodiments, R^{3b} is —C(S)R^{1a}, wherein R^{1a} is as defined herein. In certain embodiments, R^{3b} is —C(S)OR^{1a}, wherein R^{1a} is as defined herein. In certain embodiments, R^{3b} is —C(S)NR^{1b}R^{1c}, wherein R^{1b} and R^{1c} are each as defined herein. In certain embodiments, R^{3b} is —S(O)R^{1a}, wherein R^{1a} is as defined herein. In certain embodiments, R^{3b} is —S(O)₂R^{1a}, wherein R^{1a} is as defined herein. In certain embodiments, R^{3b} is —S(O)NR^{1b}R^{1c}, wherein R^{1b} and R^{1a} are each as defined herein. In certain embodiments, R^{3b} is —S(O)₂NR^{1b}R^{1c}, wherein R^{1b} and R^{c} are each as defined herein.

In certain embodiments, R^{3a} and R^{3b} together with the N atom to which they are attached form heterocyclyl, optionally substituted with one, two, or three substituents Q. In certain embodiments, R^{3a} and R^{3b} together with the N atom to which they are attached form monocyclic heterocyclyl, optionally substituted with one, two, or three substituents Q. In certain embodiments, R^{3a} and R^{3b} together with the N atom to which they are attached form 3-, 4-, 5-, 6-, or 7-membered heterocyclyl, each optionally substituted with one, two, or three substituents Q. In certain embodiments, R^{3a} and R^{3b} together with the N atom to which they are attached form 3-membered heterocyclyl, optionally substituted with one, two, or three substituents Q. In certain embodiments, R^{3a} and R^{3b} together with the N atom to which they are attached form 4-membered heterocyclyl, optionally substituted with one, two, or three substituents Q. In certain embodiments, R^{3a} and R^{3b} together with the N atom to which they are attached form 5-membered heterocyclyl, optionally substituted with one, two, or three substituents Q. In certain embodiments, R^{3a} and R^{3b} together with the N atom to which they are attached form 6-membered heterocyclyl, optionally substituted with one, two, or three substituents Q. In certain embodiments, R^{3a} and R^{3b} together with the N atom to which they are attached form 7-membered heterocyclyl, optionally substituted with one, two, or three substituents Q. In certain embodiments, R^{3a} and R^{3b} together with the N atom to which they are attached form bicyclic heterocyclyl, optionally substituted with one, two, or three substituents Q. In certain embodiments, R^{3a} and R^{3b} together with the N atom to which they are attached form bridged, fused, or spiro heterocyclyl, each optionally substituted with one, two, or three substituents Q. In certain embodiments, R^{3a} and R^{3b} together with the N atom to which they are attached form bridged heterocyclyl, optionally substituted with one, two, or three substituents Q. In certain embodiments, R^{3a} and R^{3b} together with the N atom to which they are attached form fused heterocyclyl, optionally substituted with one, two, or three substituents Q. In certain embodiments, R^{3a} and R^{3b} together with the N atom to which they are attached form spiro heterocyclyl, optionally substituted with one, two, or three substituents Q.

In certain embodiments, R^{4a} is —C(O)R^{1a}, wherein R^{1a} is as defined herein. In certain embodiments, R^{4a} is —C(O)OR^{1a}, wherein R^{1a} is as defined herein. In certain embodiments, R^{4a} is —C(O)NR^{1b}R^{1c}, wherein R^{1b} and R^{1a} are each as defined herein. In certain embodiments, R^{4a} is —C(O)SR^{1a}, wherein R^{1a} is as defined herein. In certain embodiments, R^{4a} is —C(NR^{1a})NR^{1b}R^{1c}, wherein R^{1a}, R^{1b}, and R^{1c} are each as defined herein. In certain embodiments, R^{4a} is —C(S)R^{1a}, wherein R^{1a} is as defined herein. In certain embodiments, R^{4a} is —C(S)OR^{1a}, wherein R^{1a} is as defined herein. In certain embodiments, R^{4a} is —C(S)NR^{1b}R^{1c}, wherein R^{1b} and R^{1a} are each as defined herein. In certain embodiments, R^{4a} is —S(O)R^{1a}, wherein R^{1a} is as defined herein. In certain embodiments, R^{4a} is —S(O)₂R^{1a}, wherein R^{1a} is as defined herein. In certain embodiments, R^{4a} is —S(O)NR^{1b}R^{1c}, wherein R^{1b} and R^{1a} are each as defined herein. In certain embodiments, R^{4a} is —S(O)₂NR^{1b}R^{1c}, wherein R^{1b} and R^{1c} are each as defined herein.

In certain embodiments, R^{4b} is hydrogen. In certain embodiments, R^{4b} is C₁₋₆ alkyl, optionally substituted with one, two, or three substituents Q. In certain embodiments, R^{4b} is C₁₋₆ heteroalkyl, optionally substituted with one, two, or three substituents Q. In certain embodiments, R^{4b} is C₂₋₆ alkenyl, optionally substituted with one, two, or three substituents Q. In certain embodiments, R^{4b} is C₂₋₆ alkynyl, optionally substituted with one, two, or three substituents Q. In certain embodiments, R^{4b} is C₃₋₁₀ cycloalkyl, optionally substituted with one, two, or three substituents Q. In certain embodiments, R^{4b} is monocyclic C₃₋₁₀ cycloalkyl, optionally substituted with one, two, or three substituents Q. In certain embodiments, R^{4b} is bicyclic C₄₋₁₀ cycloalkyl, optionally substituted with one, two, or three substituents Q. In certain embodiments, R^{4b} is bridged, fused, or spiro C₄₋₁₀ cycloalkyl, each optionally substituted with one, two, or three substituents Q. In certain embodiments, R^{4b} is bridged C₄₋₁₀ cycloalkyl, optionally substituted with one, two, or three substituents Q. In certain embodiments, R^{4b} is fused C₄₋₁₀ cycloalkyl, optionally substituted with one, two, or three substituents Q. In certain embodiments, R^{4b} is spiro C₄₋₁₀ cycloalkyl, optionally substituted with one, two, or three substituents Q. In certain embodiments, R^{4b} is C₆₋₁₄ aryl, optionally substituted with one, two, or three substituents Q. In certain embodiments, R^{4b} is phenyl, optionally substituted with one, two, or three substituents Q. In certain embodiments, R^{4b} is bicyclic C₈₋₁₄ aryl, optionally substituted with one, two, or three substituents Q. In certain embodiments, R^{4b} is C₇₋₁₅ aralkyl, optionally substituted with one, two, or three substituents Q.

In certain embodiments, R^{4b} is heteroaryl, optionally substituted with one, two, or three substituents Q. In certain embodiments, R^{4b} is monocyclic heteroaryl, optionally substituted with one, two, or three substituents Q. In certain embodiments, R^{4b} is 5- or 6-membered heteroaryl, each optionally substituted with one, two, or three substituents Q. In certain embodiments, R^{4b} is 5-membered heteroaryl, optionally substituted with one, two, or three substituents Q. In certain embodiments, R^{4b} is 6-membered heteroaryl, optionally substituted with one, two, or three substituents Q. In certain embodiments, R^{4b} is bicyclic heteroaryl, optionally substituted with one, two, or three substituents Q. In certain embodiments, R^{4b} is 5,5-, 5,6-, or 6,6-fused heteroaryl, each optionally substituted with one, two, or three substituents Q. In certain embodiments, R^{4b} is 5,5-fused heteroaryl, optionally substituted with one, two, or three substituents Q. In certain embodiments, R^{4b} is 5,6-fused heteroaryl, optionally substituted with one, two, or three substituents Q. In certain embodiments, R^{4b} is 6,6-fused heteroaryl, optionally substituted with one, two, or three substituents Q.

In certain embodiments, R^{4b} is heterocyclyl, optionally substituted with one, two, or three substituents Q. In certain embodiments, R^{4b} is monocyclic heterocyclyl, optionally substituted with one, two, or three substituents Q. In certain embodiments, R^{4b} is 3-, 4-, 5-, 6-, or 7-membered heterocyclyl, each optionally substituted with one, two, or three substituents Q. In certain embodiments, R^{4b} is 3-membered heterocyclyl, optionally substituted with one, two, or three substituents Q. In certain embodiments, R^{4b} is 4-membered heterocyclyl, optionally substituted with one, two, or three substituents Q. In certain embodiments, R^{4b} is 5-membered heterocyclyl, optionally substituted with one, two, or three substituents Q. In certain embodiments, R^{4b} is 6-membered heterocyclyl, optionally substituted with one, two, or three substituents Q. In certain embodiments, R^{4b} is 7-membered heterocyclyl, optionally substituted with one, two, or three substituents Q. In certain embodiments, R^{4b} is bicyclic heterocyclyl, optionally substituted with one, two, or three substituents Q. In certain embodiments, R^{4b} is bridged, fused, or spiro heterocyclyl, each optionally substituted with one, two, or three substituents Q. In certain embodiments, R^{4b} is bridged heterocyclyl, optionally substituted with one, two, or three substituents Q. In certain embodiments, R^{4b} is fused heterocyclyl, optionally substituted with one, two, or three substituents Q. In certain embodiments, R^{4b} is spiro heterocyclyl, optionally substituted with one, two, or three substituents Q.

In certain embodiments, R^{4b} is —C(O)R^{1a}, wherein R^{1a} is as defined herein. In certain embodiments, R^{4b} is —C(O)OR^{1a}, wherein R^{1a} is as defined herein. In certain embodiments, R^{4b} is —C(O)NR^{1b}R^{1c}, wherein R^{1b} and R^{1a} are each as defined herein. In certain embodiments, R^{4b} is —C(O)SR^{1a}, wherein R^{1a} is as defined herein. In certain embodiments, R^{4b} is —C(NR^{1a})NR^{1b}R^{1c}, wherein R^{1a}, R^{1b}, and R^{1c} are each as defined herein. In certain embodiments, R^{4b} is —C(S)R^{1a}, wherein R^{1a} is as defined herein. In certain embodiments, R^{4b} is —C(S)OR^{1a}, wherein R^{1a} is as defined herein. In certain embodiments, R^{4b} is —C(S)NR^{1b}R^{1c}, wherein R^{1b} and R^{1c} are each as defined herein. In certain embodiments, R^{4b} is —S(O)R^{1a}, wherein R^{1a} is as defined herein. In certain embodiments, R^{4b} is —S(O)₂R^{1a}, wherein R^{1a} is as defined herein. In certain embodiments, R^{4b} is —S(O)NR^{1b}R^{1c}, wherein R^{1b} and R^{1a} are each as defined herein. In certain embodiments, R^{4b} is —S(O)₂NR^{1b}R^{1c}, wherein R^{1b} and R^{1a} are each as defined herein.

In certain embodiments, R^{4a} and R^{4b} together with the N atom to which they are attached form heterocyclyl, optionally substituted with one, two, or three substituents Q. In certain embodiments, R^{4a} and R^{4b} together with the N atom to which they are attached form monocyclic heterocyclyl, optionally substituted with one, two, or three substituents Q. In certain embodiments, R^{4a} and R^{4b} together with the N atom to which they are attached form 3-, 4-, 5-, 6-, or 7-membered heterocyclyl, each optionally substituted with one, two, or three substituents Q. In certain embodiments, R^{4a} and R^{4b} together with the N atom to which they are attached form 3-membered heterocyclyl, optionally substituted with one, two, or three substituents Q. In certain embodiments, R^{4a} and R^{4b} together with the N atom to which they are attached form 4-membered heterocyclyl, optionally substituted with one, two, or three substituents Q. In certain embodiments, R^{4a} and R^{4b} together with the N atom to which they are attached form 5-membered heterocyclyl, optionally substituted with one, two, or three substituents Q. In certain embodiments, R^{4a} and R^{4b} together with the N atom to which they are attached form 6-membered heterocyclyl, optionally substituted with one, two, or three substituents Q. In certain embodiments, R^{4a} and R^{4b} together with the N atom to which they are attached form 7-membered heterocyclyl, optionally substituted with one, two, or three substituents Q. In certain embodiments, R^{4a} and R^{4b} together with the N atom to which they are attached form bicyclic heterocyclyl, optionally substituted with one, two, or three substituents Q. In certain embodiments, R^{4a} and R^{4b} together with the N atom to which they are attached form bridged, fused, or spiro heterocyclyl, each optionally substituted with one, two, or three substituents Q. In certain embodiments, R^{4a} and R^{4b} together with the N atom to which they are attached form bridged heterocyclyl, optionally substituted with one, two, or three substituents Q. In certain embodiments, R^{4a} and R^{4b} together with the N atom to which they are attached form fused heterocyclyl, optionally substituted with one, two, or three substituents Q. In certain embodiments, R^{4a} and R^{4b} together with the N atom to which they are attached form spiro heterocyclyl, optionally substituted with one, two, or three substituents Q.

In certain embodiments, A is —C(O)—. In certain embodiments, A is —C(O)C(O)—. In certain embodiments, A is —S(O)—. In certain embodiments, A is —S(O₂)—.

In certain embodiments, L³ is C₁₋₆ alkylene, optionally substituted with one, two, or three substituents Q. In certain embodiments, L³ is C₁₋₆ heteroalkylene, optionally substituted with one, two, or three substituents Q. In certain embodiments, L³ is C₃₋₁₀ cycloalkylene, optionally substituted with one, two, or three substituents Q. In certain embodiments, L³ is heterocyclylene, optionally substituted with one, two, or three substituents Q.

In certain embodiments, L⁴ is C₁₋₆ alkylene, optionally substituted with one, two, or three substituents Q. In certain embodiments, L⁴ is C₁₋₆ heteroalkylene, optionally substituted with one, two, or three substituents Q. In certain embodiments, L⁴ is C₃₋₁₀ cycloalkylene, optionally substituted with one, two, or three substituents Q. In certain embodiments, L⁴ is heterocyclylene, optionally substituted with one, two, or three substituents Q.

In certain embodiments, a is an integer of 0. In certain embodiments, a is an integer of 1. In certain embodiments, a is an integer of 2. In certain embodiments, a is an integer of 3.

In certain embodiments, b is an integer of 0. In certain embodiments, b is an integer of 1. In certain embodiments, b is an integer of 2. In certain embodiments, b is an integer of 3.

In certain embodiments, c is an integer of 0. In certain embodiments, c is an integer of 1. In certain embodiments, c is an integer of 2. In certain embodiments, c is an integer of 3.

In certain embodiments, d is an integer of 0. In certain embodiments, d is an integer of 1. In certain embodiments, d is an integer of 2. In certain embodiments, d is an integer of 3.

In certain embodiments, m is an integer of 0. In certain embodiments, m is an integer of 1. In certain embodiments, m is an integer of 2. In certain embodiments, m is an integer of 3.

In certain embodiments, n is an integer of 0. In certain embodiments, n is an integer of 1. In certain embodiments, n is an integer of 2. In certain embodiments, n is an integer of 3. In certain embodiments, n is an integer of 4. In certain embodiments, n is an integer of 5. In certain embodiments, n is an integer of 6. In certain embodiments, n is an integer of 7. In certain embodiments, n is an integer of 8.

In certain embodiments, p is an integer of 0. In certain embodiments, p is an integer of 1. In certain embodiments, p is an integer of 2. In certain embodiments, p is an integer of 3. In certain embodiments, p is an integer of 4. In certain embodiments, p is an integer of 5. In certain embodiments, p is an integer of 6. In certain embodiments, p is an integer of 7. In certain embodiments, p is an integer of 8.

In one embodiment, provided herein is a compound of:
2-((3-(2,6-dioxopiperidin-3-yl)-1-methyl-1H-indazol-6-yl)oxy)-N-(oxazol-2-yl)-acetamide A001;
2-((3-(2,6-dioxopiperidin-3-yl)-1-methyl-1H-indazol-6-yl)oxy)-N-((1s,4s)-4-hydroxycyclohexyl)acetamide A002;
2-((3-(2,6-dioxopiperidin-3-yl)-1-methyl-1H-indazol-6-yl)oxy)-N-(3-fluoro-5-morpholinophenyl)acetamide A003;
N-(4-cyano-3-methylisoxazol-5-yl)-2-((3-(2,6-dioxopiperidin-3-yl)-1-methyl-1H-indazol-6-yl)oxy)acetamide A004;
2-((3-(2,6-dioxopiperidin-3-yl)-1-methyl-1H-indazol-6-yl)oxy)-N-((1-methyl-1H-imidazol-5-yl)methyl)acetamide A005;
N-(1-benzylpyrrolidin-3-yl)-2-((3-(2,6-dioxopiperidin-3-yl)-1-methyl-1H-indazol-6-yl)oxy)acetamide A006;
N-(3-cyclopropyl-1-methyl-1H-pyrazol-5-yl)-2-((3-(2,6-dioxopiperidin-3-yl)-1-methyl-1H-indazol-6-yl)oxy)acetamide A007;
2-((3-(2,6-dioxopiperidin-3-yl)-1-methyl-1H-indazol-6-yl)oxy)-N-(2-sulfamoyl-ethyl)acetamide A008;
2-((3-(2,6-dioxopiperidin-3-yl)-1-methyl-1H-indazol-6-yl)oxy)-N-((1-hydroxy-cyclopropyl)methyl)acetamide A009;
2-((3-(2,6-dioxopiperidin-3-yl)-1-methyl-1H-indazol-6-yl)oxy)-N-(1-methyl-1H-pyrazol-4-yl)acetamide A010; 2-((3-(2,6-dioxopiperidin-3-yl)-1-methyl-1H-indazol-6-yl)oxy)-N-((1-methyl-1H-pyrazol-3-yl)methyl)acetamide A011;
N-(2-cyanoethyl)-2-((3-(2,6-dioxopiperidin-3-yl)-1-methyl-1H-indazol-6-yl)oxy)-acetamide A012;
2-((3-(2,6-dioxopiperidin-3-yl)-1-methyl-1H-indazol-6-yl)oxy)-N-(2-(p-tolyloxy)-ethyl)acetamide A013;
N-((4-benzylmorpholin-2-yl)methyl)-2-((3-(2,6-dioxopiperidin-3-yl)-1-methyl-1H-indazol-6-yl)oxy)acetamide A014;
2-((3-(2,6-dioxopiperidin-3-yl)-1-methyl-1H-indazol-6-yl)oxy)-N-(1-phenyl-1H-pyrazol-4-yl)acetamide A015;
2-((3-(2,6-dioxopiperidin-3-yl)-1-methyl-1H-indazol-6-yl)oxy)-N-(thiazol-2-yl-methyl)acetamide A016;
2-((3-(2,6-dioxopiperidin-3-yl)-1-methyl-1H-indazol-6-yl)oxy)-N-(4-((pyrrolidin-1-ylsulfonyl)methyl)phenyl)acetamide A017;
N-(3,5-dimethylisoxazol-4-yl)-2-((3-(2,6-dioxopiperidin-3-yl)-1-methyl-1H-indazol-6-yl)oxy)acetamide A018;
2-((3-(2,6-dioxopiperidin-3-yl)-1-methyl-1H-indazol-6-yl)oxy)-N-(2-(methyl-amino)-2-oxoethyl)acetamide A019;
2-((3-(2,6-dioxopiperidin-3-yl)-1-methyl-1H-indazol-6-yl)oxy)-N-(4-(N-methyl-sulfamoyl)phenyl)acetamide A020;
2-((3-(2,6-dioxopiperidin-3-yl)-1-methyl-1H-indazol-6-yl)oxy)-N-(1-methyl-1H-1,2,4-triazol-5-yl)acetamide A021;
2-((3-(2,6-dioxopiperidin-3-yl)-1-methyl-1H-indazol-6-yl)oxy)-N-(4-(methyl-sulfonyl)phenyl)acetamide A022;
2-((3-(2,6-dioxopiperidin-3-yl)-1-methyl-1H-indazol-6-yl)oxy)-N-(4-sulfamoyl-phenethyl)acetamide A023;
2-((3-(2,6-dioxopiperidin-3-yl)-1-methyl-1H-indazol-6-yl)oxy)-N-(4-(1-methyl-piperidin-4-yl)phenyl)acetamide A024;
2-((3-(2,6-dioxopiperidin-3-yl)-1-methyl-1H-indazol-6-yl)oxy)-N-((6-ethoxy-pyridin-3-yl)methyl)acetamide A025;
2-((3-(2,6-dioxopiperidin-3-yl)-1-methyl-1H-indazol-6-yl)oxy)-N-(pyridazin-3-yl)acetamide A026;
2-((3-(2,6-dioxopiperidin-3-yl)-1-methyl-1H-indazol-6-yl)oxy)-N-((4-hydroxy-tetrahydro-2H-pyran-4-yl)methyl)acetamide A027;
2-((3-(2,6-dioxopiperidin-3-yl)-1-methyl-1H-indazol-6-yl)oxy)-N-(pyrimidin-5-yl)acetamide A028;
2-((3-(2,6-dioxopiperidin-3-yl)-1-methyl-1H-indazol-6-yl)oxy)-N-(1-methyl-azetidin-3-yl)acetamide A029;
2-((3-(2,6-dioxopiperidin-3-yl)-1-methyl-1H-indazol-6-yl)oxy)-N-(3-methyl-isoxazol-5-yl)acetamide A030;
N-(4-(tert-butyl) thiazol-2-yl)-2-((3-(2,6-dioxopiperidin-3-yl)-1-methyl-1H-indazol-6-yl)oxy)acetamide A031;
2-((3-(2,6-dioxopiperidin-3-yl)-1-methyl-1H-indazol-6-yl)oxy)-N-(2-methyl-thiazol-5-yl)acetamide A032;
2-((3-(2,6-dioxopiperidin-3-yl)-1-methyl-1H-indazol-6-yl)oxy)-N-(3-ethyl-isoxazol-5-yl)acetamide A0033;
2-((3-(2,6-dioxopiperidin-3-yl)-1-methyl-1H-indazol-6-yl)oxy)-N-(3-methyl-isothiazol-5-yl)acetamide A0034;
2-((3-(2,6-dioxopiperidin-3-yl)-1-methyl-1H-indazol-6-yl)oxy)-N-(thiazol-4-yl)-acetamide A035;
2-((3-(2,6-dioxopiperidin-3-yl)-1-methyl-1H-indazol-6-yl)oxy)-N-(3-methyl-isothiazol-4-yl)acetamide A036;
N-((1,5-dimethyl-1H-pyrazol-3-yl)methyl)-2-((3-(2,6-dioxopiperidin-3-yl)-1-methyl-1H-indazol-6-yl)oxy)acetamide A037;
N-(3-(1,1-dioxidothiomorpholino) propyl)-2-((3-(2,6-dioxopiperidin-3-yl)-1-methyl-1H-indazol-6-yl)oxy)acetamide A038;
2-((3-(2,6-dioxopiperidin-3-yl)-1-methyl-1H-indazol-6-yl)oxy)-N-(tetrahydro-furan-3-yl)acetamide A039;
2-((3-(2,6-dioxopiperidin-3-yl)-1-methyl-1H-indazol-6-yl)oxy)-N—((R)-1-(pyridin-2-yl)ethyl)acetamide A040;
2-((3-(2,6-dioxopiperidin-3-yl)-1-methyl-1H-indazol-6-yl)oxy)-N-(oxetan-3-yl)-acetamide A041;
2-((3-(2,6-dioxopiperidin-3-yl)-1-methyl-1H-indazol-6-yl)oxy)-N-(4-hydroxy-benzyl)acetamide A042;
2-((3-(2,6-dioxopiperidin-3-yl)-1-methyl-1H-indazol-6-yl)oxy)-N-((2-methyl-1H-indol-5-yl)methyl)acetamide A043;
N-(4-cyanobicyclo[2.2.2]octan-1-yl)-2-((3-(2,6-dioxopiperidin-3-yl)-1-methyl-1H-indazol-6-yl)oxy)acetamide A044;
2-((3-(2,6-dioxopiperidin-3-yl)-1-methyl-1H-indazol-6-yl)oxy)-N-(4-hydroxy-phenethyl)acetamide A045;
N-((3-cyclohexyl-1,2,4-oxadiazol-5-yl)methyl)-2-((3-(2,6-dioxopiperidin-3-yl)-1-methyl-1H-indazol-6-yl)oxy)acetamide A046;
N-(3,5-dimethoxybenzyl)-2-((3-(2,6-dioxopiperidin-3-yl)-1-methyl-1H-indazol-6-yl)oxy)acetamide A047;
2-((3-(2,6-dioxopiperidin-3-yl)-1-methyl-1H-indazol-6-yl)oxy)-N-(4-isopropyl-benzyl)acetamide A048;
2-((3-(2,6-dioxopiperidin-3-yl)-1-methyl-1H-indazol-6-yl)oxy)-N-((3-hydroxy-cyclopentyl)methyl)acetamide A049;
N-(2,5-difluorobenzyl)-2-((3-(2,6-dioxopiperidin-3-yl)-1-methyl-1H-indazol-6-yl)oxy)acetamide A050;
2-((3-(2,6-dioxopiperidin-3-yl)-1-methyl-1H-indazol-6-yl)oxy)-N-((1-methyl-1H-indol-3-yl)methyl)acetamide A051;
2-((3-(2,6-dioxopiperidin-3-yl)-1-methyl-1H-indazol-6-yl)oxy)-N-neopentyl-acetamide A052;
N-(3,5-dichlorobenzyl)-2-((3-(2,6-dioxopiperidin-3-yl)-1-methyl-1H-indazol-6-yl)oxy)acetamide A053;
N-(1,1-dioxidotetrahydro-2H-thiopyran-4-yl)-2-((3-(2,6-dioxopiperidin-3-yl)-1-methyl-1H-indazol-6-yl)oxy)acetamide A054;
2-((3-(2,6-dioxopiperidin-3-yl)-1-methyl-1H-indazol-6-yl)oxy)-N-(4-methyl-tetrahydro-2H-pyran-4-yl)acetamide A055;
N-(2,4-difluorobenzyl)-2-((3-(2,6-dioxopiperidin-3-yl)-1-methyl-1H-indazol-6-yl)oxy)acetamide A056;
N-(2-(benzyloxy)ethyl)-2-((3-(2,6-dioxopiperidin-3-yl)-1-methyl-1H-indazol-6-yl)oxy)acetamide A057;
N-(1,1-dioxidothietan-3-yl)-2-((3-(2,6-dioxopiperidin-3-yl)-1-methyl-1H-indazol-6-yl)oxy)acetamide A058;
2-((3-(2,6-dioxopiperidin-3-yl)-1-methyl-1H-indazol-6-yl)oxy)-N-(1-methyl-piperidin-4-yl)acetamide A059;
N-(3-cyanobenzyl)-2-((3-(2,6-dioxopiperidin-3-yl)-1-methyl-1H-indazol-6-yl)-oxy)acetamide A060;
2-((3-(2,6-dioxopiperidin-3-yl)-1-methyl-1H-indazol-6-yl)oxy)-N-(3-hydroxy-cyclopentyl)acetamide A061;
N-(2,3-dimethyltetrahydrofuran-3-yl)-2-((3-(2,6-dioxopiperidin-3-yl)-1-methyl-1H-indazol-6-yl)oxy)acetamide A062;
N-((2-chlorothiazol-5-yl)methyl)-2-((3-(2,6-dioxopiperidin-3-yl)-1-methyl-1H-indazol-6-yl)oxy)acetamide A063;
2-((3-(2,6-dioxopiperidin-3-yl)-1-methyl-1H-indazol-6-yl)oxy)-N—((R)-1-(5-methylfuran-2-yl) propyl)acetamide A064;
2-((3-(2,6-dioxopiperidin-3-yl)-1-methyl-1H-indazol-6-yl)oxy)-N-(pyridin-2-yl)-acetamide A065;
2-((3-(2,6-dioxopiperidin-3-yl)-1-methyl-1H-indazol-6-yl)oxy)-N—((R)-2-oxo-pyrrolidin-3-yl)acetamide A066;
2-((3-(2,6-dioxopiperidin-3-yl)-1-methyl-1H-indazol-6-yl)oxy)-N-((5-phenyl-1,2,4-oxadiazol-3-yl)methyl)acetamide A067;
N-(cyclopentylmethyl)-2-((3-(2,6-dioxopiperidin-3-yl)-1-methyl-1H-indazol-6-yl)oxy)acetamide A068;
N-(2-(cyclopentyloxy)ethyl)-2-((3-(2,6-dioxopiperidin-3-yl)-1-methyl-1H-indazol-6-yl)oxy)acetamide A069;
N-cyclopropyl-2-((3-(2,6-dioxopiperidin-3-yl)-1-methyl-1H-indazol-6-yl)oxy)-acetamide A070;
N-(2-(3,5-dimethyl-1H-1,2,4-triazol-1-yl)ethyl)-2-((3-(2,6-dioxopiperidin-3-yl)-1-methyl-1H-indazol-6-yl)oxy)acetamide A071; 2-((3-(2,6-dioxopiperidin-3-yl)-1-methyl-1H-indazol-6-yl)oxy)-N-(tetrahydro-2H-pyran-4-yl)acetamide A072;
N-cyclobutyl-2-((3-(2,6-dioxopiperidin-3-yl)-1-methyl-1H-indazol-6-yl)oxy)-acetamide A073;
N—((S)-1-(4-chlorophenyl)ethyl)-2-((3-(2,6-dioxopiperidin-3-yl)-1-methyl-1H-indazol-6-yl)oxy)acetamide A074;
2-((3-(2,6-dioxopiperidin-3-yl)-1-methyl-1H-indazol-6-yl)oxy)-N-(1-isopropyl-1H-pyrazol-4-yl)acetamide A075;
2-((3-(2,6-dioxopiperidin-3-yl)-1-methyl-1H-indazol-6-yl)oxy)-N-(1-(tetrahydro-2H-pyran-4-yl)-1H-pyrazol-4-yl)acetamide A076;
2-((3-(2,6-dioxopiperidin-3-yl)-1-methyl-1H-indazol-6-yl)oxy)-N-((1-methyl-2-oxo-1,2-dihydropyridin-4-yl)methyl)acetamide A077;
2-((3-(2,6-dioxopiperidin-3-yl)-1-methyl-1H-indazol-6-yl)oxy)-N-(4-(2-methyl-1H-imidazol-1-yl)phenyl)acetamide A078;
2-((3-(2,6-dioxopiperidin-3-yl)-1-methyl-1H-indazol-6-yl)oxy)-N-(3-fluoro-bicyclo[1.1.1]pentan-1-yl)acetamide A079;
N-(4-(1H-imidazol-2-yl)phenyl)-2-((3-(2,6-dioxopiperidin-3-yl)-1-methyl-1H-indazol-6-yl)oxy)acetamide A080;
2-((3-(2,6-dioxopiperidin-3-yl)-1-methyl-1H-indazol-6-yl)oxy)-N-(4-(4-methyl-piperazin-1-yl)phenyl)acetamide A081;
2-((3-(2,6-dioxopiperidin-3-yl)-1-methyl-1H-indazol-6-yl)oxy)-N-(4-(oxazol-5-yl)phenyl)acetamide A082;
2-((3-(2,6-dioxopiperidin-3-yl)-1-methyl-1H-indazol-6-yl)oxy)-N-(1-methyl-1H-pyrazol-5-yl)acetamide A083;
2-((3-(2,6-dioxopiperidin-3-yl)-1-methyl-1H-indazol-6-yl)oxy)-N-(4-(p-tolyloxy)-phenyl)acetamide A084;
N-(3-(tert-butyl)phenyl)-2-((3-(2,6-dioxopiperidin-3-yl)-1-methyl-1H-indazol-6-yl)oxy)acetamide A085;
2-((3-(2,6-dioxopiperidin-3-yl)-1-methyl-1H-indazol-6-yl)oxy)-N-(1-(oxetan-3-yl)-1H-pyrazol-4-yl)acetamide A086;
N-(4-(1H-imidazol-5-yl)phenyl)-2-((3-(2,6-dioxopiperidin-3-yl)-1-methyl-1H-indazol-6-yl)oxy)acetamide A087;
2-((3-(2,6-dioxopiperidin-3-yl)-1-methyl-1H-indazol-6-yl)oxy)-N-(4-(trifluoro-methoxy)phenyl)acetamide A088;
2-((3-(2,6-dioxopiperidin-3-yl)-1-methyl-1H-indazol-6-yl)oxy)-N-(3-isopropoxy-phenyl)acetamide A089;
N-(5-cyclopropyl-1H-pyrazol-3-yl)-2-((3-(2,6-dioxopiperidin-3-yl)-1-methyl-1H-indazol-6-yl)oxy)acetamide A090;
N-(3-(1H-imidazol-1-yl)phenyl)-2-((3-(2,6-dioxopiperidin-3-yl)-1-methyl-1H-indazol-6-yl)oxy)acetamide A091;
N-(cyclopropylmethyl)-2-((3-(2,6-dioxopiperidin-3-yl)-1-methyl-1H-indazol-6-yl)oxy)acetamide A092;
N-cyclohexyl-2-((3-(2,6-dioxopiperidin-3-yl)-1-methyl-1H-indazol-6-yl)oxy)-acetamide A093;
N-cyclopentyl-2-((3-(2,6-dioxopiperidin-3-yl)-1-methyl-1H-indazol-6-yl)oxy)-acetamide A094;
2-((3-(2,6-dioxopiperidin-3-yl)-1-methyl-1H-indazol-6-yl)oxy)-N-(thiophen-2-ylmethyl)acetamide A095; or
2-((3-(2,6-dioxopiperidin-3-yl)-1-methyl-1H-indazol-6-yl)oxy)-N-(4-(morpholinomethyl)benzyl)acetamide A096;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.
In another embodiment, provided herein is a compound of:
N-(3-cyano-4,5-dimethyl-1H-pyrrol-2-yl)-2-((3-(2,6-dioxopiperidin-3-yl)-1-methyl-1H-indazol-7-yl)oxy)acetamide B001;
2-((3-(2,6-dioxopiperidin-3-yl)-1-methyl-1H-indazol-7-yl)oxy)-N-(4-(trifluoro-methoxy)phenyl)acetamide B002;
6-(tert-butyl)₃-ethyl 2-(2-((3-(2,6-dioxopiperidin-3-yl)-1-methyl-1H-indazol-7-yl)oxy)acetamido)-4,7-dihydrothieno[2,3-c]pyridine-3,6 (5H)-dicarboxylate B003;
diethyl 3-(2-((3-(2,6-dioxopiperidin-3-yl)-1-methyl-1H-indazol-7-yl)oxy)-acetamido)-1H-pyrrole-2,4-dicarboxylate B004;
ethyl 2-(2-((3-(2,6-dioxopiperidin-3-yl)-1-methyl-1H-indazol-7-yl)oxy)-acetamido)-4-methyl-4,5,6,7-tetrahydrobenzo[b]thiophene-3-carboxylate B005;
N-(3-cyano-1H-pyrrol-2-yl)-2-((3-(2,6-dioxopiperidin-3-yl)-1-methyl-1H-indazol-7-yl)oxy)acetamide B006;
ethyl 5-(2-((3-(2,6-dioxopiperidin-3-yl)-1-methyl-1H-indazol-7-yl)oxy)-acetamido)-3-methyl-1H-pyrazole-4-carboxylate B007;
2-((3-(2,6-dioxopiperidin-3-yl)-1-methyl-1H-indazol-7-yl)oxy)-N-(3-(pyridin-2-yl)-1H-pyrazol-4-yl)acetamide B008;
diethyl 2-(2-((3-(2,6-dioxopiperidin-3-yl)-1-methyl-1H-indazol-7-yl)oxy)-acetamido)thiophene-3,4-dicarboxylate B009;
N-(5-chloro-2-cyanophenyl)-2-((3-(2,6-dioxopiperidin-3-yl)-1-methyl-1H-indazol-7-yl)oxy)acetamide B010;
2-((3-(2,6-dioxopiperidin-3-yl)-1-methyl-1H-indazol-7-yl)oxy)-N-(4-(oxazol-5-yl)phenyl)acetamide B011;
N-(4-cyano-3-methylisothiazol-5-yl)-2-((3-(2,6-dioxopiperidin-3-yl)-1-methyl-1H-indazol-7-yl)oxy)acetamide B012;
N-(2-cyano-3-methoxyphenyl)-2-((3-(2,6-dioxopiperidin-3-yl)-1-methyl-1H-indazol-7-yl)oxy)acetamide B013;
N-(4-cyano-1-phenyl-1H-pyrazol-5-yl)-2-((3-(2,6-dioxopiperidin-3-yl)-1-methyl-1H-indazol-7-yl)oxy)acetamide B014;
N-(3-cyano-6-methyl-4,5,6,7-tetrahydrobenzo[b]thiophen-2-yl)-2-((3-(2,6-dioxo-piperidin-3-yl)-1-methyl-1H-indazol-7-yl)oxy)acetamide B015;
N-(2-cyano-5-(trifluoromethyl)phenyl)-2-((3-(2,6-dioxopiperidin-3-yl)-1-methyl-1H-indazol-7-yl)oxy)acetamide B016;
N-(2-cyano-3-methylphenyl)-2-((3-(2,6-dioxopiperidin-3-yl)-1-methyl-1H-indazol-7-yl)oxy)acetamide B017;
N-(2-cyano-4-methoxyphenyl)-2-((3-(2,6-dioxopiperidin-3-yl)-1-methyl-1H-indazol-7-yl)oxy)acetamide B018;
methyl 4-(2-((3-(2,6-dioxopiperidin-3-yl)-1-methyl-1H-indazol-7-yl)oxy)-acetamido)-2-methylthiophene-3-carboxylate B019;
ethyl 5-(2-((3-(2,6-dioxopiperidin-3-yl)-1-methyl-1H-indazol-7-yl)oxy)-acetamido) thiazole-4-carboxylate B020;
ethyl 2-(2-((3-(2,6-dioxopiperidin-3-yl)-1-methyl-1H-indazol-7-yl)oxy)-acetamido)-4-isopropylthiophene-3-carboxylate B021;
N-(3-cyano-4-methylthiophen-2-yl)-2-((3-(2,6-dioxopiperidin-3-yl)-1-methyl-1H-indazol-7-yl)oxy)acetamide B022;
N-(3-(2-chlorobenzoyl)-5-ethylthiophen-2-yl)-2-((3-(2,6-dioxopiperidin-3-yl)-1-methyl-1H-indazol-7-yl)oxy)acetamide B023;
N-(2-cyano-6-methylphenyl)-2-((3-(2,6-dioxopiperidin-3-yl)-1-methyl-1H-indazol-7-yl)oxy)acetamide B024;
N-(3-cyano-4-methyl-1H-pyrrol-2-yl)-2-((3-(2,6-dioxopiperidin-3-yl)-1-methyl-1H-indazol-7-yl)oxy)acetamide B025;
ethyl 5-cyano-4-(2-((3-(2,6-dioxopiperidin-3-yl)-1-methyl-1H-indazol-7-yl)oxy)-acetamido)-2-(methylthio)thiophene-3-carboxylate B026;
N-(5-(tert-butyl)-1,3,4-thiadiazol-2-yl)-2-((3-(2,6-dioxopiperidin-3-yl)-1-methyl-1H-indazol-7-yl)oxy)acetamide B027;
ethyl 2-(2-((3-(2,6-dioxopiperidin-3-yl)-1-methyl-1H-indazol-7-yl)oxy)-acetamido)-4-phenylthiophene-3-carboxylate B028;
methyl 2-(2-((3-(2,6-dioxopiperidin-3-yl)-1-methyl-1H-indazol-7-yl)oxy)-acetamido)-1H-indole-3-carboxylate B029;
2-((3-(2,6-dioxopiperidin-3-yl)-1-methyl-1H-indazol-7-yl)oxy)-N-(2-hydroxy-2,3-dihydro-1H-inden-1-yl)acetamide B030;
N-(4-cyano-3-methyl-1-phenyl-1H-pyrazol-5-yl)-2-((3-(2,6-dioxopiperidin-3-yl)-1-methyl-1H-indazol-7-yl)oxy)acetamide B031;
3-cyano-4-(2-((3-(2,6-dioxopiperidin-3-yl)-1-methyl-1H-indazol-7-yl)oxy)-acetamido)benzoic acid B032;
2-((3-(2,6-dioxopiperidin-3-yl)-1-methyl-1H-indazol-7-yl)oxy)-N-(4-(trifluoro-methyl) thiazol-2-yl)acetamide B033;
N-(3-bromo-2-cyanophenyl)-2-((3-(2,6-dioxopiperidin-3-yl)-1-methyl-1H-indazol-7-yl)oxy)acetamide B034;
2-((3-(2,6-dioxopiperidin-3-yl)-1-methyl-1H-indazol-7-yl)oxy)-N-(thiazol-4-yl-methyl)acetamide B035;
methyl 5-(2-((3-(2,6-dioxopiperidin-3-yl)-1-methyl-1H-indazol-7-yl)oxy)-acetamido)-3-methylthiophene-2-carboxylate B036;
N-(3-cyano-5-(trifluoromethyl)phenyl)-2-((3-(2,6-dioxopiperidin-3-yl)-1-methyl-1H-indazol-7-yl)oxy)acetamide B037;
2-((3-(2,6-dioxopiperidin-3-yl)-1-methyl-1H-indazol-7-yl)oxy)-N-(3-methyl-isothiazol-5-yl)acetamide B038;
methyl 2-(2-((3-(2,6-dioxopiperidin-3-yl)-1-methyl-1H-indazol-7-yl)oxy)-acetamido)-4,5-dimethylthiophene-3-carboxylate B039;
N-(2-cyanopyridin-3-yl)-2-((3-(2,6-dioxopiperidin-3-yl)-1-methyl-1H-indazol-7-yl)oxy)acetamide B040;
2-((3-(2,6-dioxopiperidin-3-yl)-1-methyl-1H-indazol-7-yl)oxy)-N-(3-phenyl-1H-pyrazol-4-yl)acetamide B041;
N-(3-cyano-4,5-dimethylthiophen-2-yl)-2-((3-(2,6-dioxopiperidin-3-yl)-1-methyl-1H-indazol-7-yl)oxy)acetamide B042;
methyl 2-(2-((3-(2,6-dioxopiperidin-3-yl)-1-methyl-1H-indazol-7-yl)oxy)-acetamido)-4,5,6,7-tetrahydrobenzo[b]thiophene-3-carboxylate B043;
5-cyano-2-(2-((3-(2,6-dioxopiperidin-3-yl)-1-methyl-1H-indazol-7-yl)oxy)-acetamido)benzoic acid B044;
methyl 3-(2-((3-(2,6-dioxopiperidin-3-yl)-1-methyl-1H-indazol-7-yl)oxy)-acetamido)-5-iodothiophene-2-carboxylate B045;
N-(2-cyano-3-fluorophenyl)-2-((3-(2,6-dioxopiperidin-3-yl)-1-methyl-1H-indazol-7-yl)oxy)acetamide B046;
2-((3-(2,6-dioxopiperidin-3-yl)-1-methyl-1H-indazol-7-yl)oxy)-N-(4-methyl-1,2,5-oxadiazol-3-yl)acetamide B047;
N-(2-cyano-4-fluorophenyl)-2-((3-(2,6-dioxopiperidin-3-yl)-1-methyl-1H-indazol-7-yl)oxy)acetamide B048;
N-(2-cyano-5-methylpyridin-3-yl)-2-((3-(2,6-dioxopiperidin-3-yl)-1-methyl-1H-indazol-7-yl)oxy)acetamide B049;
N-(2-cyanothiophen-3-yl)-2-((3-(2,6-dioxopiperidin-3-yl)-1-methyl-1H-indazol-7-yl)oxy)acetamide B050;
N-(2-cyano-4,6-difluorophenyl)-2-((3-(2,6-dioxopiperidin-3-yl)-1-methyl-1H-indazol-7-yl)oxy)acetamide B051;
N-(2-cyano-4-methylphenyl)-2-((3-(2,6-dioxopiperidin-3-yl)-1-methyl-1H-indazol-7-yl)oxy)acetamide B052;
N-(2-cyanobenzyl)-2-((3-(2,6-dioxopiperidin-3-yl)-1-methyl-1H-indazol-7-yl)-oxy)acetamide B053;
N-(4-cyano-1,3-dimethyl-1H-pyrazol-5-yl)-2-((3-(2,6-dioxopiperidin-3-yl)-1-methyl-1H-indazol-7-yl)oxy)acetamide B054;
methyl 3-(2-((3-(2,6-dioxopiperidin-3-yl)-1-methyl-1H-indazol-7-yl)oxy)-acetamido)-2H-thieno[3,2-c]pyrazole-5-carboxylate B055;
N-(2-cyanonaphthalen-1-yl)-2-((3-(2,6-dioxopiperidin-3-yl)-1-methyl-1H-indazol-7-yl)oxy)acetamide B056;
N-(6-chloro-3-cyanopyridin-2-yl)-2-((3-(2,6-dioxopiperidin-3-yl)-1-methyl-1H-indazol-7-yl)oxy)acetamide B057;
N-(3-cyanothiophen-2-yl)-2-((3-(2,6-dioxopiperidin-3-yl)-1-methyl-1H-indazol-7-yl)oxy)acetamide B058;
N-(2-cyano-5-methylphenyl)-2-((3-(2,6-dioxopiperidin-3-yl)-1-methyl-1H-indazol-7-yl)oxy)acetamide B059;
2-((3-(2,6-dioxopiperidin-3-yl)-1-methyl-1H-indazol-7-yl)oxy)-N-(1-methyl-3-(trifluoromethyl)-1H-pyrazol-5-yl)acetamide B060;
N-(4-(3-chlorophenyl)-1H-pyrazol-5-yl)-2-((3-(2,6-dioxopiperidin-3-yl)-1-methyl-1H-indazol-7-yl)oxy)acetamide B061;
N-(2-cyano-6-fluorophenyl)-2-((3-(2,6-dioxopiperidin-3-yl)-1-methyl-1H-indazol-7-yl)oxy)acetamide B062;
ethyl 2-(2-((3-(2,6-dioxopiperidin-3-yl)-1-methyl-1H-indazol-7-yl)oxy)-acetamido)-4,5,6,7-tetrahydrobenzo[b]thiophene-3-carboxylate B063;
N-((1-cyanocyclopentyl)methyl)-2-((3-(2,6-dioxopiperidin-3-yl)-1-methyl-1H-indazol-7-yl)oxy)acetamide B064;
N-(4-cyano-1-methyl-1H-pyrazol-5-yl)-2-((3-(2,6-dioxopiperidin-3-yl)-1-methyl-1H-indazol-7-yl)oxy)acetamide B065;
N-(4-cyano-3-methylisoxazol-5-yl)-2-((3-(2,6-dioxopiperidin-3-yl)-1-methyl-1H-indazol-7-yl)oxy)acetamide B066;
N-(1-(tert-butyl)-3-methyl-1H-pyrazol-5-yl)-2-((3-(2,6-dioxopiperidin-3-yl)-1-methyl-1H-indazol-7-yl)oxy)acetamide B067;
2-((3-(2,6-dioxopiperidin-3-yl)-1-methyl-1H-indazol-7-yl)oxy)-N-(5-(furan-2-yl)-1,3,4-oxadiazol-2-yl)acetamide B068;
2-((3-(2,6-dioxopiperidin-3-yl)-1-methyl-1H-indazol-7-yl)oxy)-N-(5-methyl-1,3,4-oxadiazol-2-yl)acetamide B069;
N-(3,5-dimethylisoxazol-4-yl)-2-((3-(2,6-dioxopiperidin-3-yl)-1-methyl-1H-indazol-7-yl)oxy)acetamide B070;
2-((3-(2,6-dioxopiperidin-3-yl)-1-methyl-1H-indazol-7-yl)oxy)-N-((4-hydroxy-tetrahydro-2H-pyran-4-yl)methyl)acetamide B071;
2-((3-(2,6-dioxopiperidin-3-yl)-1-methyl-1H-indazol-7-yl)oxy)-N-(5-ethyl-isoxazol-3-yl)acetamide B072;
2-((3-(2,6-dioxopiperidin-3-yl)-1-methyl-1H-indazol-7-yl)oxy)-N-(3-ethyl-isoxazol-5-yl)acetamide B073;
2-((3-(2,6-dioxopiperidin-3-yl)-1-methyl-1H-indazol-7-yl)oxy)-N-(3-methyl-isoxazol-5-yl)acetamide B074;
2-((3-(2,6-dioxopiperidin-3-yl)-1-methyl-1H-indazol-7-yl)oxy)-N-(isoxazol-5-yl)acetamide B075;
2-((3-(2,6-dioxopiperidin-3-yl)-1-methyl-1H-indazol-7-yl)oxy)-N-(1-methyl-1H-1,2,4-triazol-5-yl)acetamide B076;
2-((3-(2,6-dioxopiperidin-3-yl)-1-methyl-1H-indazol-7-yl)oxy)-N-((8-methyl-[1,2,4]triazolo[4,3-a]pyridin-3-yl)methyl)acetamide B077;
2-((3-(2,6-dioxopiperidin-3-yl)-1-methyl-1H-indazol-7-yl)oxy)-N-(4-(2-methyl-1H-imidazol-1-yl)phenyl)acetamide B078;
2-((3-(2,6-dioxopiperidin-3-yl)-1-methyl-1H-indazol-7-yl)oxy)-N-((6-ethoxy-pyridin-3-yl)methyl)acetamide B079;
2-((3-(2,6-dioxopiperidin-3-yl)-1-methyl-1H-indazol-7-yl)oxy)-N—(((R)-5-oxo-pyrrolidin-2-yl)methyl)acetamide B080;
2-((3-(2,6-dioxopiperidin-3-yl)-1-methyl-1H-indazol-7-yl)oxy)-N-(1-methyl-2-oxopiperidin-4-yl)acetamide B081;
2-((3-(2,6-dioxopiperidin-3-yl)-1-methyl-1H-indazol-7-yl)oxy)-N-(2-(5-isopropyl-1,2,4-oxadiazol-3-yl)ethyl)acetamide B082;
2-((3-(2,6-dioxopiperidin-3-yl)-1-methyl-1H-indazol-7-yl)oxy)-N-(4-(1-methyl-piperidin-4-yl)phenyl)acetamide B083;
2-((3-(2,6-dioxopiperidin-3-yl)-1-methyl-1H-indazol-7-yl)oxy)-N-(4-(N-methyl-sulfamoyl)phenyl)acetamide B084;
2-((3-(2,6-dioxopiperidin-3-yl)-1-methyl-1H-indazol-7-yl)oxy)-N—((R)-2-oxo-azepan-3-yl)acetamide B085;
N-(4-(1H-imidazol-2-yl)phenyl)-2-((3-(2,6-dioxopiperidin-3-yl)-1-methyl-1H-indazol-7-yl)oxy)acetamide B086;
2-((3-(2,6-dioxopiperidin-3-yl)-1-methyl-1H-indazol-7-yl)oxy)-N-(thiazol-4-yl)-acetamide B087;
2-((3-(2,6-dioxopiperidin-3-yl)-1-methyl-1H-indazol-7-yl)oxy)-N-(4-(methyl-sulfonyl)phenyl)acetamide B088; 2-((3-(2,6-dioxopiperidin-3-yl)-1-methyl-1H-indazol-7-yl)oxy)-N-(pyridazin-3-yl)acetamide B089;
2-((3-(2,6-dioxopiperidin-3-yl)-1-methyl-1H-indazol-7-yl)oxy)-N-(4-(4-methyl-piperazin-1-yl)phenyl)acetamide B090;
2-((3-(2,6-dioxopiperidin-3-yl)-1-methyl-1H-indazol-7-yl)oxy)-N—((R)-1-(pyridin-2-yl)ethyl)acetamide B091;
2-((3-(2,6-dioxopiperidin-3-yl)-1-methyl-1H-indazol-7-yl)oxy)-N-(oxazol-2-yl)-acetamide B092;
2-((3-(2,6-dioxopiperidin-3-yl)-1-methyl-1H-indazol-7-yl)oxy)-N-(4-(2-oxo-pyrrolidin-1-yl)benzyl)acetamide B093;
2-((3-(2,6-dioxopiperidin-3-yl)-1-methyl-1H-indazol-7-yl)oxy)-N-(oxetan-3-yl)-acetamide B094;
2-((3-(2,6-dioxopiperidin-3-yl)-1-methyl-1H-indazol-7-yl)oxy)-N-(1-methyl-1H-pyrazol-4-yl)acetamide B095;
2-((3-(2,6-dioxopiperidin-3-yl)-1-methyl-1H-indazol-7-yl)oxy)-N—(((S)-oxetan-2-yl)methyl)acetamide B096;
N-((2-chlorothiazol-5-yl)methyl)-2-((3-(2,6-dioxopiperidin-3-yl)-1-methyl-1H-indazol-7-yl)oxy)acetamide B097;
2-((3-(2,6-dioxopiperidin-3-yl)-1-methyl-1H-indazol-7-yl)oxy)-N—((R)-1-(4-fluorophenyl)ethyl)acetamide B098;
2-((3-(2,6-dioxopiperidin-3-yl)-1-methyl-1H-indazol-7-yl)oxy)-N—((S)-1-phenyl-propyl)acetamide B099;
N—((S)-1-(4-chlorophenyl)ethyl)-2-((3-(2,6-dioxopiperidin-3-yl)-1-methyl-1H-indazol-7-yl)oxy)acetamide B100;
2-((3-(2,6-dioxopiperidin-3-yl)-1-methyl-1H-indazol-7-yl)oxy)-N-(1-phenyl-1H-pyrazol-4-yl)acetamide B101;
2-((3-(2,6-dioxopiperidin-3-yl)-1-methyl-1H-indazol-7-yl)oxy)-N—((S)-1-(p-tolyl)-ethyl)acetamide B102;
N-(3,5-dimethyl-1H-pyrazol-4-yl)-2-((3-(2,6-dioxopiperidin-3-yl)-1-methyl-1H-indazol-7-yl)oxy)acetamide B103;
N-(3-(difluoromethoxy)-1H-pyrazol-5-yl)-2-((3-(2,6-dioxopiperidin-3-yl)-1-methyl-1H-indazol-7-yl)oxy)acetamide B104;
2-((3-(2,6-dioxopiperidin-3-yl)-1-methyl-1H-indazol-7-yl)oxy)-N-(2-(methyl-amino)-2-oxoethyl)acetamide B105;
2-((3-(2,6-dioxopiperidin-3-yl)-1-methyl-1H-indazol-7-yl)oxy)-N-((1s,4s)-4-hydroxycyclohexyl)acetamide B106;
2-((3-(2,6-dioxopiperidin-3-yl)-1-methyl-1H-indazol-7-yl)oxy)-N-(2-oxo-piperidin-3-yl)acetamide B107;
2-((3-(2,6-dioxopiperidin-3-yl)-1-methyl-1H-indazol-7-yl)oxy)-N-((1r,4r)-4-methoxycyclohexyl)acetamide B108;
N-(3-(tert-butyl)-1-phenyl-1H-pyrazol-5-yl)-2-((3-(2,6-dioxopiperidin-3-yl)-1-methyl-1H-indazol-7-yl)oxy)acetamide B109;
N-(4-(1H-pyrrol-1-yl)phenyl)-2-((3-(2,6-dioxopiperidin-3-yl)-1-methyl-1H-indazol-7-yl)oxy)acetamide B110;
2-((3-(2,6-dioxopiperidin-3-yl)-1-methyl-1H-indazol-7-yl)oxy)-N-(4-hydroxy-phenethyl)acetamide B111;
2-((3-(2,6-dioxopiperidin-3-yl)-1-methyl-1H-indazol-7-yl)oxy)-N-((2-methyl-1H-indol-5-yl)methyl)acetamide B112;
2-((3-(2,6-dioxopiperidin-3-yl)-1-methyl-1H-indazol-7-yl)oxy)-N-(2-methyl-thiazol-5-yl)acetamide B113;
2-((3-(2,6-dioxopiperidin-3-yl)-1-methyl-1H-indazol-7-yl)oxy)-N-(pyridin-2-yl)-acetamide B114;
N-(4-(tert-butyl) thiazol-2-yl)-2-((3-(2,6-dioxopiperidin-3-yl)-1-methyl-1H-indazol-7-yl)oxy)acetamide B115;
2-((3-(2,6-dioxopiperidin-3-yl)-1-methyl-1H-indazol-7-yl)oxy)-N-((3-hydroxy-cyclopentyl)methyl)acetamide B116;
2-((3-(2,6-dioxopiperidin-3-yl)-1-methyl-1H-indazol-7-yl)oxy)-N-(3-methyl-isothiazol-4-yl)acetamide B117;
2-((3-(2,6-dioxopiperidin-3-yl)-1-methyl-1H-indazol-7-yl)oxy)-N-(4-((pyrrolidin-1-ylsulfonyl)methyl)phenyl)acetamide B118;
N-(4-(sec-butoxy)benzyl)-2-((3-(2,6-dioxopiperidin-3-yl)-1-methyl-1H-indazol-7-yl)oxy)acetamide B119;
2-((3-(2,6-dioxopiperidin-3-yl)-1-methyl-1H-indazol-7-yl)oxy)-N-(1-methyl-piperidin-4-yl)acetamide B120;
2-((3-(2,6-dioxopiperidin-3-yl)-1-methyl-1H-indazol-7-yl)oxy)-N-(3-hydroxy-cyclopentyl)acetamide B121;
2-((3-(2,6-dioxopiperidin-3-yl)-1-methyl-1H-indazol-7-yl)oxy)-N-(3-fluoro-5-morpholinophenyl)acetamide B122;
N-(2-(3,5-dimethyl-1H-1,2,4-triazol-1-yl)ethyl)-2-((3-(2,6-dioxopiperidin-3-yl)-1-methyl-1H-indazol-7-yl)oxy)acetamide B123;
N-(4-cyano-1-(2-hydroxyethyl)-1H-pyrazol-5-yl)-2-((3-(2,6-dioxopiperidin-3-yl)-1-methyl-1H-indazol-7-yl)oxy)acetamide B124;
2-((3-(2,6-dioxopiperidin-3-yl)-1-methyl-1H-indazol-7-yl)oxy)-N-((1-methyl-1H-imidazol-5-yl)methyl)acetamide B125;
N-(1,1-dioxidotetrahydro-2H-thiopyran-4-yl)-2-((3-(2,6-dioxopiperidin-3-yl)-1-methyl-1H-indazol-7-yl)oxy)acetamide B126;
N-(4-cyanobicyclo[2.2.2]octan-1-yl)-2-((3-(2,6-dioxopiperidin-3-yl)-1-methyl-1H-indazol-7-yl)oxy)acetamide B127;
N-(3-cyanobenzyl)-2-((3-(2,6-dioxopiperidin-3-yl)-1-methyl-1H-indazol-7-yl)-oxy)acetamide B128;
2-((3-(2,6-dioxopiperidin-3-yl)-1-methyl-1H-indazol-7-yl)oxy)-N-((1-methyl-2-oxo-1,2-dihydropyridin-4-yl)methyl)acetamide B129;
N-(3-cyclopropyl-1-methyl-1H-pyrazol-5-yl)-2-((3-(2,6-dioxopiperidin-3-yl)-1-methyl-1H-indazol-7-yl)oxy)acetamide B130;
2-((3-(2,6-dioxopiperidin-3-yl)-1-methyl-1H-indazol-7-yl)oxy)-N-(4-(p-tolyloxy)-phenyl)acetamide B131;
2-((3-(2,6-dioxopiperidin-3-yl)-1-methyl-1H-indazol-7-yl)oxy)-N-((1-hydroxy-cyclopropyl)methyl)acetamide B132;
2-((3-(2,6-dioxopiperidin-3-yl)-1-methyl-1H-indazol-7-yl)oxy)-N-((5-methyl-isoxazol-3-yl)methyl)acetamide B133;
2-((3-(2,6-dioxopiperidin-3-yl)-1-methyl-1H-indazol-7-yl)oxy)-N-((1-methyl-1H-indol-3-yl)methyl)acetamide B134;
2-((3-(2,6-dioxopiperidin-3-yl)-1-methyl-1H-indazol-7-yl)oxy)-N-(1-methyl-pyrrolidin-3-yl)acetamide B135;
N-(2-(benzyloxy)ethyl)-2-((3-(2,6-dioxopiperidin-3-yl)-1-methyl-1H-indazol-7-yl)oxy)acetamide B136;
N-(1-benzylpyrrolidin-3-yl)-2-((3-(2,6-dioxopiperidin-3-yl)-1-methyl-1H-indazol-7-yl)oxy)acetamide B137;
2-((3-(2,6-dioxopiperidin-3-yl)-1-methyl-1H-indazol-7-yl)oxy)-N-(4-isopropyl-benzyl)acetamide B138;
2-((3-(2,6-dioxopiperidin-3-yl)-1-methyl-1H-indazol-7-yl)oxy)-N-(tetrahydro-furan-3-yl)acetamide B139;
2-((3-(2,6-dioxopiperidin-3-yl)-1-methyl-1H-indazol-7-yl)oxy)-N-(1-methyl-azetidin-3-yl)acetamide B140;
2-((3-(2,6-dioxopiperidin-3-yl)-1-methyl-1H-indazol-7-yl)oxy)-N-(1-methyl-1H-pyrazol-5-yl)acetamide B141;
2-((3-(2,6-dioxopiperidin-3-yl)-1-methyl-1H-indazol-7-yl)oxy)-N-((3-methyl-1,2,4-oxadiazol-5-yl)methyl)acetamide B142;
2-((3-(2,6-dioxopiperidin-3-yl)-1-methyl-1H-indazol-7-yl)oxy)-N-(4-sulfamoyl-phenethyl)acetamide B143;
2-((3-(2,6-dioxopiperidin-3-yl)-1-methyl-1H-indazol-7-yl)oxy)-N-((tetrahydro-furan-2-yl)methyl)acetamide B144;
2-((3-(2,6-dioxopiperidin-3-yl)-1-methyl-1H-indazol-7-yl)oxy)-N-((1-methyl-1H-pyrazol-3-yl)methyl)acetamide B145;
2-((3-(2,6-dioxopiperidin-3-yl)-1-methyl-1H-indazol-7-yl)oxy)-N-(1-propyl-piperidin-4-yl)acetamide B146;
N-((3-cyclohexyl-1,2,4-oxadiazol-5-yl)methyl)-2-((3-(2,6-dioxopiperidin-3-yl)-1-methyl-1H-indazol-7-yl)oxy)acetamide B147;
2-((3-(2,6-dioxopiperidin-3-yl)-1-methyl-1H-indazol-7-yl)oxy)-N-((5-phenyl-1,2,4-oxadiazol-3-yl)methyl)acetamide B148;
2-((3-(2,6-dioxopiperidin-3-yl)-1-methyl-1H-indazol-7-yl)oxy)-N-(3-fluoro-bicyclo[1.1.1]pentan-1-yl)acetamide B149;
2-((3-(2,6-dioxopiperidin-3-yl)-1-methyl-1H-indazol-7-yl)oxy)-N-(1-isopropyl-1H-pyrazol-4-yl)acetamide B150;
2-((3-(2,6-dioxopiperidin-3-yl)-1-methyl-1H-indazol-7-yl)oxy)-N-(2-(p-tolyloxy)-ethyl)acetamide B151;
N-(1,1-dioxidothietan-3-yl)-2-((3-(2,6-dioxopiperidin-3-yl)-1-methyl-1H-indazol-7-yl)oxy)acetamide B152;
N-(3,5-dimethoxybenzyl)-2-((3-(2,6-dioxopiperidin-3-yl)-1-methyl-1H-indazol-7-yl)oxy)acetamide B153;
N-(2,5-difluorobenzyl)-2-((3-(2,6-dioxopiperidin-3-yl)-1-methyl-1H-indazol-7-yl)oxy)acetamide B154;
2-((3-(2,6-dioxopiperidin-3-yl)-1-methyl-1H-indazol-7-yl)oxy)-N-((1-methyl-pyrrolidin-3-yl)methyl)acetamide B155;
N-((4-benzylmorpholin-2-yl)methyl)-2-((3-(2,6-dioxopiperidin-3-yl)-1-methyl-1H-indazol-7-yl)oxy)acetamide B156;
N-((1,5-dimethyl-1H-pyrazol-3-yl)methyl)-2-((3-(2,6-dioxopiperidin-3-yl)-1-methyl-1H-indazol-7-yl)oxy)acetamide B157;
2-((3-(2,6-dioxopiperidin-3-yl)-1-methyl-1H-indazol-7-yl)oxy)-N-neopentyl-acetamide B158;
2-((3-(2,6-dioxopiperidin-3-yl)-1-methyl-1H-indazol-7-yl)oxy)-N-(3-methyl-1H-pyrazol-5-yl)acetamide B159;
2-((3-(2,6-dioxopiperidin-3-yl)-1-methyl-1H-indazol-7-yl)oxy)-N-(4-(morpholinomethyl)benzyl)acetamide B160;
2-((3-(2,6-dioxopiperidin-3-yl)-1-methyl-1H-indazol-7-yl)oxy)-N-(tetrahydro-2H-pyran-4-yl)acetamide B161;
N-(3,5-dichlorobenzyl)-2-((3-(2,6-dioxopiperidin-3-yl)-1-methyl-1H-indazol-7-yl)oxy)acetamide B162;
N-(3,5-difluorobenzyl)-2-((3-(2,6-dioxopiperidin-3-yl)-1-methyl-1H-indazol-7-yl)oxy)acetamide B163;
2-((3-(2,6-dioxopiperidin-3-yl)-1-methyl-1H-indazol-7-yl)oxy)-N-(oxetan-2-yl-methyl)acetamide B164;
N-(2-(cyclopentyloxy)ethyl)-2-((3-(2,6-dioxopiperidin-3-yl)-1-methyl-1H-indazol-7-yl)oxy)acetamide B165;
N-(cyclopentylmethyl)-2-((3-(2,6-dioxopiperidin-3-yl)-1-methyl-1H-indazol-7-yl)oxy)acetamide B166;
N-cyclobutyl-2-((3-(2,6-dioxopiperidin-3-yl)-1-methyl-1H-indazol-7-yl)oxy)-acetamide B167;
2-((3-(2,6-dioxopiperidin-3-yl)-1-methyl-1H-indazol-7-yl)oxy)-N-(thiazol-2-yl-methyl)acetamide B168;
N-(4-(1H-pyrazol-1-yl)pyridin-2-yl)-2-((3-(2,6-dioxopiperidin-3-yl)-1-methyl-1H-indazol-7-yl)oxy)acetamide B169;
2-((3-(2,6-dioxopiperidin-3-yl)-1-methyl-1H-indazol-7-yl)oxy)-N-(3-isopropoxy-phenyl)acetamide B170;
2-((3-(2,6-dioxopiperidin-3-yl)-1-methyl-1H-indazol-7-yl)oxy)-N-(3-(oxazol-5-yl)phenyl)acetamide B171;
2-((3-(2,6-dioxopiperidin-3-yl)-1-methyl-1H-indazol-7-yl)oxy)-N-(4-hydroxy-benzyl)acetamide B172;
2-((3-(2,6-dioxopiperidin-3-yl)-1-methyl-1H-indazol-7-yl)oxy)-N-(thiophen-2-yl-methyl)acetamide B173;
N-cyclopropyl-2-((3-(2,6-dioxopiperidin-3-yl)-1-methyl-1H-indazol-7-yl)oxy)-acetamide B174;
N-(3-(tert-butyl)phenyl)-2-((3-(2,6-dioxopiperidin-3-yl)-1-methyl-1H-indazol-7-yl)oxy)acetamide B175;
N-(cyclopropylmethyl)-2-((3-(2,6-dioxopiperidin-3-yl)-1-methyl-1H-indazol-7-yl)oxy)acetamide B176;
N-(3-cyclopropyl-1H-pyrazol-5-yl)-2-((3-(2,6-dioxopiperidin-3-yl)-1-methyl-1H-indazol-7-yl)oxy)acetamide B177;
N-(3-(1H-imidazol-1-yl)phenyl)-2-((3-(2,6-dioxopiperidin-3-yl)-1-methyl-1H-indazol-7-yl)oxy)acetamide B178; or
N-cyclohexyl-2-((3-(2,6-dioxopiperidin-3-yl)-1-methyl-1H-indazol-7-yl)oxy)-acetamide B179;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.
In yet another embodiment, provided herein is a compound of:
4-amino-5-(4-(2-((3-(2,6-dioxopiperidin-3-yl)-1-methyl-1H-indazol-7-yl)oxy)-acetyl)piperazine-1-carbonyl) isothiazole-3-carboxamide B201;
5-(4-(2-((3-(2,6-dioxopiperidin-3-yl)-1-methyl-1H-indazol-7-yl)oxy)acetyl)-piperazine-1-carbonyl)-1H-pyrrole-2-carboxylic acid B202;
3-(7-(2-(4-((1,2-dimethyl-1H-imidazol-5-yl) sulfonyl)piperazin-1-yl)-2-oxo-ethoxy)-1-methyl-1H-indazol-3-yl)piperidine-2,6-dione B203;
3-(4-(2-((3-(2,6-dioxopiperidin-3-yl)-1-methyl-1H-indazol-7-yl)oxy)acetyl)-piperazine-1-carbonyl)-1-methyl-1H-pyrazole-5-carboxylic acid B204;
3-(7-(2-(4-((5-(5-chloro-1,2,4-thiadiazol-3-yl)thiophen-2-yl) sulfonyl)piperazin-1-yl)-2-oxoethoxy)-1-methyl-1H-indazol-3-yl)piperidine-2,6-dione B205;
4-amino-5-(4-(2-((3-(2,6-dioxopiperidin-3-yl)-1-methyl-1H-indazol-7-yl)oxy)-acetyl)piperazine-1-carbonyl)-N-isopropylisothiazole-3-carboxamide B206;
3-(1-methyl-7-(2-oxo-2-(4-((tetrahydro-2H-pyran-4-yl) sulfonyl)piperazin-1-yl)-ethoxy)-1H-indazol-3-yl)piperidine-2,6-dione B207;
3-(1-methyl-7-(2-(4-((1-methyl-1H-imidazol-4-yl) sulfonyl)piperazin-1-yl)-2-oxo-ethoxy)-1H-indazol-3-yl)piperidine-2,6-dione B208;
4-amino-5-(4-(2-((3-(2,6-dioxopiperidin-3-yl)-1-methyl-1H-indazol-7-yl)oxy)-acetyl)piperazine-1-carbonyl)-N-ethylisothiazole-3-carboxamide B209;
3-(1-methyl-7-(2-oxo-2-(4-((1,3,5-trimethyl-1H-pyrazol-4-yl) sulfonyl)piperazin-1-yl)ethoxy)-1H-indazol-3-yl)piperidine-2,6-dione B210;
3-(1-methyl-7-(2-oxo-2-(4-((2,2,4,6,7-pentamethyl-2,3-dihydrobenzofuran-5-yl)-sulfonyl)piperazin-1-yl)ethoxy)-1H-indazol-3-yl)piperidine-2,6-dione B211;
3-(7-(2-(4-(2-(cyclopropylamino) thiazole-5-carbonyl)piperazin-1-yl)-2-oxo-ethoxy)-1-methyl-1H-indazol-3-yl)piperidine-2,6-dione B212;
3-(1-methyl-7-(2-(4-(3-methylisothiazole-5-carbonyl)piperazin-1-yl)-2-oxo-ethoxy)-1H-indazol-3-yl)piperidine-2,6-dione B213;
3-(7-(2-(4-((1-ethyl-3-methyl-1H-pyrazol-5-yl) sulfonyl)piperazin-1-yl)-2-oxo-ethoxy)-1-methyl-1H-indazol-3-yl)piperidine-2,6-dione B214;
3-(7-(2-(4-((3,5-dimethylisoxazol-4-yl) sulfonyl)piperazin-1-yl)-2-oxoethoxy)-1-methyl-1H-indazol-3-yl)piperidine-2,6-dione B215;
3-(7-(2-(4-((1-ethyl-1H-pyrazol-4-yl) sulfonyl)piperazin-1-yl)-2-oxoethoxy)-1-methyl-1H-indazol-3-yl)piperidine-2,6-dione B216;
3-(7-(2-(4-((cyclobutylmethyl) sulfonyl)piperazin-1-yl)-2-oxoethoxy)-1-methyl-1H-indazol-3-yl)piperidine-2,6-dione B217;
3-(1-methyl-7-(2-(4-((3-(5-methyl-1,2,4-oxadiazol-3-yl)phenyl) sulfonyl)-piperazin-1-yl)-2-oxoethoxy)-1H-indazol-3-yl)piperidine-2,6-dione B218;
3-(7-(2-(4-(1,2,4-oxadiazole-3-carbonyl)piperazin-1-yl)-2-oxoethoxy)-1-methyl-1H-indazol-3-yl)piperidine-2,6-dione B219;
3-(7-(2-(4-(6,7-dihydro-4H-pyrazolo[5,1-c][1,4]oxazine-2-carbonyl)piperazin-1-yl)-2-oxoethoxy)-1-methyl-1H-indazol-3-yl)piperidine-2,6-dione B220;
3-(7-(2-(4-(2,4-dimethyloxazole-5-carbonyl)piperazin-1-yl)-2-oxoethoxy)-1-methyl-1H-indazol-3-yl)piperidine-2,6-dione B221;
3-(1-methyl-7-(2-oxo-2-(4-((4-(trifluoromethoxy)phenyl) sulfonyl)piperazin-1-yl)ethoxy)-1H-indazol-3-yl)piperidine-2,6-dione B222;
3-(1-methyl-7-(2-(4-(4-methylthiazole-2-carbonyl)piperazin-1-yl)-2-oxoethoxy)-1H-indazol-3-yl)piperidine-2,6-dione B223;
5-(4-(2-((3-(2,6-dioxopiperidin-3-yl)-1-methyl-1H-indazol-7-yl)oxy)acetyl)-piperazine-1-carbonyl)-1H-pyrrole-3-carbonitrile B224;
3-(1-methyl-7-(2-(4-(2-methyl-3H-pyrazolo[1,5-b][1,2,4]triazole-7-carbonyl)-piperazin-1-yl)-2-oxoethoxy)-1H-indazol-3-yl)piperidine-2,6-dione B225;
methyl 5-(4-(2-((3-(2,6-dioxopiperidin-3-yl)-1-methyl-1H-indazol-7-yl)oxy)-acetyl)piperazine-1-carbonyl)-1-methyl-1H-pyrazole-3-carboxylate B226;
ethyl 5-(4-(2-((3-(2,6-dioxopiperidin-3-yl)-1-methyl-1H-indazol-7-yl)oxy)-acetyl)piperazine-1-carbonyl)-1H-pyrrole-2-carboxylate B227;
3-(7-(2-(4-(2,5-dimethyloxazole-4-carbonyl)piperazin-1-yl)-2-oxoethoxy)-1-methyl-1H-indazol-3-yl)piperidine-2,6-dione B228;
3-(1-methyl-7-(2-oxo-2-(4-(2,4,5,6-tetrahydrocyclopenta[c]pyrrole-1-carbonyl)-piperazin-1-yl)ethoxy)-1H-indazol-3-yl)piperidine-2,6-dione B229;
3-(1-methyl-7-(2-oxo-2-(4-((3-oxo-3,4-dihydro-2H-benzo[b][1,4]oxazin-6-yl)-sulfonyl)piperazin-1-yl)ethoxy)-1H-indazol-3-yl)piperidine-2,6-dione B230;
3-(1-methyl-7-(2-oxo-2-(4-((2-oxoindolin-6-yl) sulfonyl)piperazin-1-yl)ethoxy)-1H-indazol-3-yl)piperidine-2,6-dione B231;
3-(7-(2-(4-((2-chloro-5-methylpyridin-3-yl) sulfonyl)piperazin-1-yl)-2-oxo-ethoxy)-1-methyl-1H-indazol-3-yl)piperidine-2,6-dione B232;
3-(7-(2-(4-(4-amino-1,2,5-oxadiazole-3-carbonyl)piperazin-1-yl)-2-oxoethoxy)-1-methyl-1H-indazol-3-yl)piperidine-2,6-dione B233;
4-((4-(2-((3-(2,6-dioxopiperidin-3-yl)-1-methyl-1H-indazol-7-yl)oxy)acetyl)-piperazin-1-yl) sulfonyl)benzamide B234;
3-(7-(2-(4-((4-(tert-butyl)phenyl) sulfonyl)piperazin-1-yl)-2-oxoethoxy)-1-methyl-1H-indazol-3-yl)piperidine-2,6-dione B235;
ethyl 4-(4-(2-((3-(2,6-dioxopiperidin-3-yl)-1-methyl-1H-indazol-7-yl)oxy)acetyl)-piperazine-1-carbonyl)-1H-pyrrole-2-carboxylate B236;
3-(7-(2-(4-((1,5-dimethyl-1H-pyrazol-3-yl) sulfonyl)piperazin-1-yl)-2-oxo-ethoxy)-1-methyl-1H-indazol-3-yl)piperidine-2,6-dione B237;
3-(1-methyl-7-(2-(4-((1-methyl-1H-pyrazol-4-yl) sulfonyl)piperazin-1-yl)-2-oxo-ethoxy)-1H-indazol-3-yl)piperidine-2,6-dione B238;
3-(1-methyl-7-(2-(4-(5-methylthiazole-2-carbonyl)piperazin-1-yl)-2-oxoethoxy)-1H-indazol-3-yl)piperidine-2,6-dione B239;
3-(7-(2-(4-((4-fluorophenyl) sulfonyl)piperazin-1-yl)-2-oxoethoxy)-1-methyl-1H-indazol-3-yl)piperidine-2,6-dione B240;
3-(1-methyl-7-(2-oxo-2-(4-(thiophen-2-ylsulfonyl)piperazin-1-yl)ethoxy)-1H-indazol-3-yl)piperidine-2,6-dione B241;
3-(7-(2-(4-(4-amino-1-methyl-1H-pyrazole-5-carbonyl)piperazin-1-yl)-2-oxo-ethoxy)-1-methyl-1H-indazol-3-yl)piperidine-2,6-dione B242; or
5-(4-(2-((3-(2,6-dioxopiperidin-3-yl)-1-methyl-1H-indazol-7-yl)oxy)acetyl)-piperazine-1-carbonyl) thiazole-2-carbonitrile B243;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is a compound of:
3-(1-methyl-7-((1-(oxetane-3-carbonyl)piperidin-4-yl)oxy)-1H-indazol-3-yl)-piperidine-2,6-dione C001;
3-(7-((1-(4-amino-1-methyl-1H-pyrazole-3-carbonyl)piperidin-4-yl)oxy)-1-methyl-1H-indazol-3-yl)piperidine-2,6-dione C002;
3-(1-methyl-7-((1-(2-(4-oxo-2-thioxothiazolidin-3-yl)acetyl)piperidin-4-yl)oxy)-1H-indazol-3-yl)piperidine-2,6-dione C003;
3-(1-methyl-7-((1-(4-oxo-1,4-dihydropyrrolo[1,2-b]pyridazine-3-carbonyl)-piperidin-4-yl)oxy)-1H-indazol-3-yl)piperidine-2,6-dione C004;
3-(1-methyl-7-((1-(3-(4-(trifluoromethyl)phenyl) propioloyl)piperidin-4-yl)oxy)-1H-indazol-3-yl)piperidine-2,6-dione C005;
5-(4-((3-(2,6-dioxopiperidin-3-yl)-1-methyl-1H-indazol-7-yl)oxy) piperidine-1-carbonyl)-1H-pyrrole-2-carboxamide C006;
3-(7-((1-(4-amino-1-methyl-1H-pyrazole-5-carbonyl)piperidin-4-yl)oxy)-1-methyl-1H-indazol-3-yl)piperidine-2,6-dione C007;
3-(1-methyl-7-((1-(methylglycyl)piperidin-4-yl)oxy)-1H-indazol-3-yl)piperidine-2,6-dione C008;
2-(4-((3-(2,6-dioxopiperidin-3-yl)-1-methyl-1H-indazol-7-yl)oxy) piperidine-1-carbonyl)-1H-pyrrole-3-carbonitrile C009;
3-(7-((1-(azetidine-3-carbonyl)piperidin-4-yl)oxy)-1-methyl-1H-indazol-3-yl)-piperidine-2,6-dione C010;
(E)-3-(1-methyl-7-((1-(3-(4-(trifluoromethyl)phenyl) acryloyl)piperidin-4-yl)oxy)-1H-indazol-3-yl)piperidine-2,6-dione C011;
ethyl 4-(4-((3-(2,6-dioxopiperidin-3-yl)-1-methyl-1H-indazol-7-yl)oxy)-piperidine-1-carbonyl)-1H-pyrrole-2-carboxylate C012;
3-(1-methyl-7-((1-(2-phenylthiazole-4-carbonyl)piperidin-4-yl)oxy)-1H-indazol-3-yl)piperidine-2,6-dione C013;
3-(7-((1-(5-methoxypicolinoyl)piperidin-4-yl)oxy)-1-methyl-1H-indazol-3-yl)-piperidine-2,6-dione C014;
3-(7-((1-(2-((2,4-difluorophenyl)thio) acetyl)piperidin-4-yl)oxy)-1-methyl-1H-indazol-3-yl)piperidine-2,6-dione C015;
4-(4-((3-(2,6-dioxopiperidin-3-yl)-1-methyl-1H-indazol-7-yl)oxy) piperidine-1-carbonyl)-3-fluorobenzonitrile C016;
3-(1-methyl-7-((1-(1-methyl-3-(trifluoromethyl)-1H-pyrazole-4-carbonyl)-piperidin-4-yl)oxy)-1H-indazol-3-yl)piperidine-2,6-dione C017;
3-(1-methyl-7-((1-(2-((phenylthio)methyl)benzoyl)piperidin-4-yl)oxy)-1H-indazol-3-yl)piperidine-2,6-dione C018;
3-(1-methyl-7-((1-(1-methyl-5-(trifluoromethyl)-1H-pyrrole-2-carbonyl)-piperidin-4-yl)oxy)-1H-indazol-3-yl)piperidine-2,6-dione C019;
3-(7-((1-(3-methoxythiophene-2-carbonyl)piperidin-4-yl)oxy)-1-methyl-1H-indazol-3-yl)piperidine-2,6-dione C020;
3-(1-methyl-7-((1-(pyrrolo[2,1-b]thiazole-6-carbonyl)piperidin-4-yl)oxy)-1H-indazol-3-yl)piperidine-2,6-dione C021;
3-(7-((1-(4,5-dimethylthiophene-3-carbonyl)piperidin-4-yl)oxy)-1-methyl-1H-indazol-3-yl)piperidine-2,6-dione C022;
3-(1-methyl-7-((1-(1-methyl-1H-pyrrole-2-carbonyl)piperidin-4-yl)oxy)-1H-indazol-3-yl)piperidine-2,6-dione C023;
3-(7-((1-(2,2-difluoro-2-(4-fluorophenyl)acetyl)piperidin-4-yl)oxy)-1-methyl-1H-indazol-3-yl)piperidine-2,6-dione C024;
3-(7-((1-(2-cyclobutylacetyl)piperidin-4-yl)oxy)-1-methyl-1H-indazol-3-yl)-piperidine-2,6-dione C025;
3-(7-((1-(bicyclo[1.1.1]pentane-1-carbonyl)piperidin-4-yl)oxy)-1-methyl-1H-indazol-3-yl)piperidine-2,6-dione C026;
3-(7-((1-(4-isopropoxybenzoyl)piperidin-4-yl)oxy)-1-methyl-1H-indazol-3-yl)-piperidine-2,6-dione C027;
3-(7-((1-(1,1-dioxidothietane-3-carbonyl)piperidin-4-yl)oxy)-1-methyl-1H-indazol-3-yl)piperidine-2,6-dione C028;
3-(7-((1-(imidazo[1,2-a]pyridine-6-carbonyl)piperidin-4-yl)oxy)-1-methyl-1H-indazol-3-yl)piperidine-2,6-dione C029;
3-(1-methyl-7-((1-(4-phenoxybenzoyl)piperidin-4-yl)oxy)-1H-indazol-3-yl)-piperidine-2,6-dione C030;
3-(1-methyl-7-((1-(4-oxo-4H-chromene-2-carbonyl)piperidin-4-yl)oxy)-1H-indazol-3-yl)piperidine-2,6-dione C031;
3-(1-methyl-7-((1-(3-phenylpropioloyl)piperidin-4-yl)oxy)-1H-indazol-3-yl)-piperidine-2,6-dione C032;
3-(7-((1-(2,4-dimethylthiazole-5-carbonyl)piperidin-4-yl)oxy)-1-methyl-1H-indazol-3-yl)piperidine-2,6-dione C033;
3-(1-methyl-7-((1-(2-(p-tolylthio) acetyl)piperidin-4-yl)oxy)-1H-indazol-3-yl)-piperidine-2,6-dione C034;
2-(4-((3-(2,6-dioxopiperidin-3-yl)-1-methyl-1H-indazol-7-yl)oxy) piperidin-1-yl)-N-(2-methoxyphenyl)-2-oxoacetamide C035;
3-(7-((1-(1,5-dimethyl-1H-pyrazole-4-carbonyl)piperidin-4-yl)oxy)-1-methyl-1H-indazol-3-yl)piperidine-2,6-dione C036;
3-(7-((1-(1-hydroxycyclopropane-1-carbonyl)piperidin-4-yl)oxy)-1-methyl-1H-indazol-3-yl)piperidine-2,6-dione C037;
3-(7-((1-(3-(difluoromethyl)-5-fluoro-1-methyl-1H-pyrazole-4-carbonyl)-piperidin-4-yl)oxy)-1-methyl-1H-indazol-3-yl)piperidine-2,6-dione C038;
N-(2-(4-((3-(2,6-dioxopiperidin-3-yl)-1-methyl-1H-indazol-7-yl)oxy) piperidin-1-yl)-2-oxoethyl)-2-phenylacetamide C039;
3-(1-methyl-7-((1-(4,5,6,7-tetrahydro-1H-indazole-3-carbonyl)piperidin-4-yl)-oxy)-1H-indazol-3-yl)piperidine-2,6-dione C040;
3-(1-methyl-7-((1-(6-methylbenzo[b]thiophene-2-carbonyl)piperidin-4-yl)oxy)-1H-indazol-3-yl)piperidine-2,6-dione C041;
3-(7-((1-(1-(hydroxymethyl)cyclopropane-1-carbonyl)piperidin-4-yl)oxy)-1-methyl-1H-indazol-3-yl)piperidine-2,6-dione C042;
3-(1-methyl-7-((1-(3-phenoxybenzoyl)piperidin-4-yl)oxy)-1H-indazol-3-yl)-piperidine-2,6-dione C043;
3-(7-((1-(3-hydroxy-4-methoxybenzoyl)piperidin-4-yl)oxy)-1-methyl-1H-indazol-3-yl)piperidine-2,6-dione C044;
3-(7-((1-(4′-fluoro-[1,1′-biphenyl]-3-carbonyl)piperidin-4-yl)oxy)-1-methyl-1H-indazol-3-yl)piperidine-2,6-dione C045;
3-(7-((1-(5-amino-1-phenyl-1H-pyrazole-4-carbonyl)piperidin-4-yl)oxy)-1-methyl-1H-indazol-3-yl)piperidine-2,6-dione C046;
3-(7-((1-(6,7-dihydro-4H-pyrazolo[5,1-c][1,4]oxazine-2-carbonyl)piperidin-4-yl)-oxy)-1-methyl-1H-indazol-3-yl)piperidine-2,6-dione C047;
4-amino-5-(4-((3-(2,6-dioxopiperidin-3-yl)-1-methyl-1H-indazol-7-yl)oxy)-piperidine-1-carbonyl) isothiazole-3-carboxamide C048;
3-(1-methyl-7-((1-(5-(pyridin-3-yl)-1H-imidazole-4-carbonyl)piperidin-4-yl)oxy)-1H-indazol-3-yl)piperidine-2,6-dione C049;
3-(1-methyl-7-((1-(1-methyl-4-oxo-1,4-dihydropyrrolo[1,2-b]pyridazine-3-carbonyl)piperidin-4-yl)oxy)-1H-indazol-3-yl)piperidine-2,6-dione C050;
3-(7-((1-(6-ethoxypicolinoyl)piperidin-4-yl)oxy)-1-methyl-1H-indazol-3-yl)-piperidine-2,6-dione C051;
3-(1-methyl-7-((1-(2,4,5,6-tetrahydrocyclopenta[c]pyrrole-1-carbonyl)piperidin-4-yl)oxy)-1H-indazol-3-yl)piperidine-2,6-dione C052;
3-amino-5-(4-((3-(2,6-dioxopiperidin-3-yl)-1-methyl-1H-indazol-7-yl)oxy)-piperidine-1-carbonyl)benzonitrile C053;
3-(7-((1-(2-(benzyloxy)-2-methylpropanoyl)piperidin-4-yl)oxy)-1-methyl-1H-indazol-3-yl)piperidine-2,6-dione C054;
3-(7-((1-(1H-pyrazole-4-carbonyl)piperidin-4-yl)oxy)-1-methyl-1H-indazol-3-yl)-piperidine-2,6-dione C055;
3-(7-((1-(²H-indazole-5-carbonyl)piperidin-4-yl)oxy)-1-methyl-1H-indazol-3-yl)-piperidine-2,6-dione C056;
3-(7-((1-(1-(tert-butyl)-1H-1,2,3-triazole-4-carbonyl)piperidin-4-yl)oxy)-1-methyl-1H-indazol-3-yl)piperidine-2,6-dione C057;
3-(1-methyl-7-((1-(5-(pyridin-3-yl)-1H-pyrazole-3-carbonyl)piperidin-4-yl)oxy)-1H-indazol-3-yl)piperidine-2,6-dione C058;
3-(7-((1-(2-mercapto-1-methyl-1H-imidazole-5-carbonyl)piperidin-4-yl)oxy)-1-methyl-1H-indazol-3-yl)piperidine-2,6-dione C059;
3-(7-((1-(3-(benzo[d][1,3]dioxol-5-yl) acryloyl)piperidin-4-yl)oxy)-1-methyl-1H-indazol-3-yl)piperidine-2,6-dione C060;
3-(7-((1-(4-amino-1,2,5-oxadiazole-3-carbonyl)piperidin-4-yl)oxy)-1-methyl-1H-indazol-3-yl)piperidine-2,6-dione C061;
3-(7-((1-(2-aminothiazole-4-carbonyl)piperidin-4-yl)oxy)-1-methyl-1H-indazol-3-yl)piperidine-2,6-dione C062;
3-(7-((1-(4-benzyl-5-oxomorpholine-3-carbonyl)piperidin-4-yl)oxy)-1-methyl-1H-indazol-3-yl)piperidine-2,6-dione C063;
3-(7-((1-(imidazo[2,1-b]thiazole-5-carbonyl)piperidin-4-yl)oxy)-1-methyl-1H-indazol-3-yl)piperidine-2,6-dione C064;
3-(1-methyl-7-((1-(3-(phenylsulfonyl) propanoyl)piperidin-4-yl)oxy)-1H-indazol-3-yl)piperidine-2,6-dione C065;
5-(4-((3-(2,6-dioxopiperidin-3-yl)-1-methyl-1H-indazol-7-yl)oxy) piperidine-1-carbonyl) pyrazine-2-carbonitrile C066;
3-(7-((1-(4-hydroxycyclohexane-1-carbonyl)piperidin-4-yl)oxy)-1-methyl-1H-indazol-3-yl)piperidine-2,6-dione C067;
3-(1-methyl-7-((1-(4,5,6,7-tetrahydrobenzo[b]thiophene-2-carbonyl)piperidin-4-yl)oxy)-1H-indazol-3-yl)piperidine-2,6-dione C068;
3-(7-((1-(4,6-dihydroxypyridazine-3-carbonyl)piperidin-4-yl)oxy)-1-methyl-1H-indazol-3-yl)piperidine-2,6-dione C069;
3-(7-((1-(2,3-dihydrobenzofuran-7-carbonyl)piperidin-4-yl)oxy)-1-methyl-1H-indazol-3-yl)piperidine-2,6-dione C070;
3-(7-((1-(6-methoxy-1H-indazole-3-carbonyl)piperidin-4-yl)oxy)-1-methyl-1H-indazol-3-yl)piperidine-2,6-dione C071;
(E)-3-(7-((1-(3-(3,4-difluorophenyl) acryloyl)piperidin-4-yl)oxy)-1-methyl-1H-indazol-3-yl)piperidine-2,6-dione C072;
3-(1-methyl-7-((1-(3-phenylcyclobutane-1-carbonyl)piperidin-4-yl)oxy)-1H-indazol-3-yl)piperidine-2,6-dione C073;
3-(1-methyl-7-((1-(2-methyl-1H-imidazole-5-carbonyl)piperidin-4-yl)oxy)-1H-indazol-3-yl)piperidine-2,6-dione C074;
3-(7-((1-(2-(cyclopropylamino) thiazole-5-carbonyl)piperidin-4-yl)oxy)-1-methyl-1H-indazol-3-yl)piperidine-2,6-dione C075;
5-(4-((3-(2,6-dioxopiperidin-3-yl)-1-methyl-1H-indazol-7-yl)oxy) piperidine-1-carbonyl) furan-3-carbonitrile C076;
3-(7-((1-(2-cyclopropylacetyl)piperidin-4-yl)oxy)-1-methyl-1H-indazol-3-yl)-piperidine-2,6-dione C077;
4-(4-((3-(2,6-dioxopiperidin-3-yl)-1-methyl-1H-indazol-7-yl)oxy) piperidine-1-carbonyl)piperidine-1-carboximidamide C078;
N-cyclohexyl-2-(4-((3-(2,6-dioxopiperidin-3-yl)-1-methyl-1H-indazol-7-yl)oxy)-piperidin-1-yl)-2-oxoacetamide C079;
5-(4-((3-(2,6-dioxopiperidin-3-yl)-1-methyl-1H-indazol-7-yl)oxy) piperidine-1-carbonyl)-1H-pyrrole-3-carbonitrile C080;
3-(7-((1-(4-(1H-imidazol-2-yl)benzoyl)piperidin-4-yl)oxy)-1-methyl-1H-indazol-3-yl)piperidine-2,6-dione C081;
3-(7-((1-(5-amino-2-methylthiazole-4-carbonyl)piperidin-4-yl)oxy)-1-methyl-1H-indazol-3-yl)piperidine-2,6-dione C082;
N-(4-(4-((3-(2,6-dioxopiperidin-3-yl)-1-methyl-1H-indazol-7-yl)oxy) piperidine-1-carbonyl)phenyl)benzenesulfonamide C083;
3-(7-((1-(cyclobut-1-ene-1-carbonyl)piperidin-4-yl)oxy)-1-methyl-1H-indazol-3-yl)piperidine-2,6-dione C084;
3-(1-methyl-7-((1-(4-oxo-4,5,6,7-tetrahydropyrazolo[1,5-a]pyrazine-2-carbonyl)-piperidin-4-yl)oxy)-1H-indazol-3-yl)piperidine-2,6-dione C085;
3-(7-((1-(3-(3-fluorophenyl) propioloyl)piperidin-4-yl)oxy)-1-methyl-1H-indazol-3-yl)piperidine-2,6-dione C086;
3-(7-((1-(4H-thieno[3,2-b]pyrrole-5-carbonyl)piperidin-4-yl)oxy)-1-methyl-1H-indazol-3-yl)piperidine-2,6-dione C087;
3-(1-methyl-7-((1-(2-methyl-4-(trifluoromethyl) furan-3-carbonyl)piperidin-4-yl)-oxy)-1H-indazol-3-yl)piperidine-2,6-dione C088;
3-(7-((1-(2,5-dimethyloxazole-4-carbonyl)piperidin-4-yl)oxy)-1-methyl-1H-indazol-3-yl)piperidine-2,6-dione C089;
3-(7-((1-(2-(2-mercapto-4-methylthiazol-5-yl)acetyl)piperidin-4-yl)oxy)-1-methyl-1H-indazol-3-yl)piperidine-2,6-dione C090;
3-(7-((1-(4-hydroxy-3-(trifluoromethyl)benzoyl)piperidin-4-yl)oxy)-1-methyl-1H-indazol-3-yl)piperidine-2,6-dione C091;
3-(7-((1-(1H-pyrrolo[2,3-b]pyridine-3-carbonyl)piperidin-4-yl)oxy)-1-methyl-1H-indazol-3-yl)piperidine-2,6-dione C092;
4-(4-((3-(2,6-dioxopiperidin-3-yl)-1-methyl-1H-indazol-7-yl)oxy) piperidine-1-carbonyl)tetrahydro-2H-pyran-4-carbonitrile C093;
3-(7-((1-(3-hydroxypropanoyl)piperidin-4-yl)oxy)-1-methyl-1H-indazol-3-yl)-piperidine-2,6-dione C094;
3-(1-methyl-7-((1-(pent-2-ynoyl)piperidin-4-yl)oxy)-1H-indazol-3-yl)piperidine-2,6-dione C095;
3-(7-((1-(2-isopropylisonicotinoyl)piperidin-4-yl)oxy)-1-methyl-1H-indazol-3-yl)piperidine-2,6-dione C096;
2-(2-(4-((3-(2,6-dioxopiperidin-3-yl)-1-methyl-1H-indazol-7-yl)oxy) piperidin-1-yl)-2-oxoethoxy)benzonitrile C097;
3-(7-((1-(6,7-dihydro-5H-pyrazolo[5,1-b][1,3]oxazine-2-carbonyl)piperidin-4-yl)oxy)-1-methyl-1H-indazol-3-yl)piperidine-2,6-dione C098;
3-(1-methyl-7-((1-(4-oxo-4H-chromene-3-carbonyl)piperidin-4-yl)oxy)-1H-indazol-3-yl)piperidine-2,6-dione C099;
3-(1-methyl-7-((1-(3-methylisothiazole-5-carbonyl)piperidin-4-yl)oxy)-1H-indazol-3-yl)piperidine-2,6-dione C100;
3-(7-((1-(2-((4-chlorophenyl)thio) acetyl)piperidin-4-yl)oxy)-1-methyl-1H-indazol-3-yl)piperidine-2,6-dione C101;
3-(7-((1-(imidazo[1,2-a]pyridine-3-carbonyl)piperidin-4-yl)oxy)-1-methyl-1H-indazol-3-yl)piperidine-2,6-dione C102;
3-(1-methyl-7-((1-(5-methylthiophene-2-carbonyl)piperidin-4-yl)oxy)-1H-indazol-3-yl)piperidine-2,6-dione C103;
3-(7-((1-(5-isopropyl-1H-pyrazole-3-carbonyl)piperidin-4-yl)oxy)-1-methyl-1H-indazol-3-yl)piperidine-2,6-dione C104;
3-(7-((1-(4-(tert-butyl)benzoyl)piperidin-4-yl)oxy)-1-methyl-1H-indazol-3-yl)-piperidine-2,6-dione C105;
3-(1-methyl-7-((1-(4,5,6,7-tetrahydrobenzo[b]thiophene-3-carbonyl)piperidin-4-yl)oxy)-1H-indazol-3-yl)piperidine-2,6-dione C106;
3-(1-methyl-7-((1-(thiazole-5-carbonyl)piperidin-4-yl)oxy)-1H-indazol-3-yl)-piperidine-2,6-dione C107;
3-(1-methyl-7-((1-(oxazole-4-carbonyl)piperidin-4-yl)oxy)-1H-indazol-3-yl)-piperidine-2,6-dione C108;
(E)-3-(7-((1-(3-(4-aminophenyl) acryloyl)piperidin-4-yl)oxy)-1-methyl-1H-indazol-3-yl)piperidine-2,6-dione C109;
3-(7-((1-(1-(6-(cyclopropylamino)pyrimidin-4-yl)piperidine-4-carbonyl)-piperidin-4-yl)oxy)-1-methyl-1H-indazol-3-yl)piperidine-2,6-dione C110;
3-(1-methyl-7-((1-(oxazole-2-carbonyl)piperidin-4-yl)oxy)-1H-indazol-3-yl)-piperidine-2,6-dione C111;
2-(4-((3-(2,6-dioxopiperidin-3-yl)-1-methyl-1H-indazol-7-yl)oxy) piperidin-1-yl)-N-(4-methoxyphenyl)-2-oxoacetamide C112;
3-(7-((1-(1,2,4-oxadiazole-3-carbonyl)piperidin-4-yl)oxy)-1-methyl-1H-indazol-3-yl)piperidine-2,6-dione C113;
3-(1-methyl-7-((1-(2-(piperazin-1-yl)acetyl)piperidin-4-yl)oxy)-1H-indazol-3-yl)-piperidine-2,6-dione C114;
(E)-3-(1-methyl-7-((1-(3-(thiazol-2-yl) acryloyl)piperidin-4-yl)oxy)-1H-indazol-3-yl)piperidine-2,6-dione C115;
(E)-3-(1-methyl-7-((1-(3-(5-methylthiophen-2-yl) acryloyl)piperidin-4-yl)oxy)-1H-indazol-3-yl)piperidine-2,6-dione C116;
1-(4-((3-(2,6-dioxopiperidin-3-yl)-1-methyl-1H-indazol-7-yl)oxy) piperidine-1-carbonyl)cyclobutane-1-carbonitrile C117;
3-(4-((3-(2,6-dioxopiperidin-3-yl)-1-methyl-1H-indazol-7-yl)oxy) piperidine-1-carbonyl)-5-methylbenzonitrile C118;
2-(4-((3-(2,6-dioxopiperidin-3-yl)-1-methyl-1H-indazol-7-yl)oxy) piperidin-1-yl)-N-(3-methoxyphenyl)-2-oxoacetamide C119;
3-(7-((1-(5-chlorofuran-2-carbonyl)piperidin-4-yl)oxy)-1-methyl-1H-indazol-3-yl)piperidine-2,6-dione C120;
3-(1-methyl-7-((1-(2-(3-methylisoxazol-5-yl)acetyl)piperidin-4-yl)oxy)-1H-indazol-3-yl)piperidine-2,6-dione C121;
(E)-3-(1-methyl-7-((1-(pent-2-enoyl)piperidin-4-yl)oxy)-1H-indazol-3-yl)-piperidine-2,6-dione C122;
(E)-3-(7-((1-(3-(benzofuran-2-yl) acryloyl)piperidin-4-yl)oxy)-1-methyl-1H-indazol-3-yl)piperidine-2,6-dione C123;
3-(1-methyl-7-((1-(1-methylpiperidine-2-carbonyl)piperidin-4-yl)oxy)-1H-indazol-3-yl)piperidine-2,6-dione C124;
3-(7-((1-(4-(1H-pyrazol-1-yl)benzoyl)piperidin-4-yl)oxy)-1-methyl-1H-indazol-3-yl)piperidine-2,6-dione C125;
3-(7-((1-(2-(1,1-dioxidothiomorpholino) acetyl)piperidin-4-yl)oxy)-1-methyl-1H-indazol-3-yl)piperidine-2,6-dione C126;
1-(3-(4-((3-(2,6-dioxopiperidin-3-yl)-1-methyl-1H-indazol-7-yl)oxy) piperidin-1-yl)-3-oxopropyl) guanidine C127;
1-(2-(4-((3-(2,6-dioxopiperidin-3-yl)-1-methyl-1H-indazol-7-yl)oxy) piperidin-1-yl)-2-oxoethyl) guanidine Cl28;
3-(1-methyl-7-((1-(4-methylthiazole-2-carbonyl)piperidin-4-yl)oxy)-1H-indazol-3-yl)piperidine-2,6-dione C129;
3-(4-((3-(2,6-dioxopiperidin-3-yl)-1-methyl-1H-indazol-7-yl)oxy) piperidine-1-carbonyl) bicyclo[1.1.1]pentane-1-carbonitrile C130;
3-(7-((1-(2-amino-4-methylthiazole-5-carbonyl)piperidin-4-yl)oxy)-1-methyl-1H-indazol-3-yl)piperidine-2,6-dione C131;
3-(1-methyl-7-((1-(4-nitrobenzoyl)piperidin-4-yl)oxy)-1H-indazol-3-yl)-piperidine-2,6-dione C132;
3-(7-((1-(1-(2-fluorophenyl)-1H-pyrazole-4-carbonyl)piperidin-4-yl)oxy)-1-methyl-1H-indazol-3-yl)piperidine-2,6-dione C133;
3-(1-methyl-7-((1-(pyridazine-3-carbonyl)piperidin-4-yl)oxy)-1H-indazol-3-yl)-piperidine-2,6-dione C134;
4-amino-5-(4-((3-(2,6-dioxopiperidin-3-yl)-1-methyl-1H-indazol-7-yl)oxy)-piperidine-1-carbonyl)-N-ethylisothiazole-3-carboxamide C135;
4-amino-5-(4-((3-(2,6-dioxopiperidin-3-yl)-1-methyl-1H-indazol-7-yl)oxy)-piperidine-1-carbonyl)-N-isopropylisothiazole-3-carboxamide C136;
3-(1-methyl-7-((1-(2-(trifluoromethoxy)benzoyl)piperidin-4-yl)oxy)-1H-indazol-3-yl)piperidine-2,6-dione C137;
3-(7-((1-(2-(1H-pyrazol-1-yl)acetyl)piperidin-4-yl)oxy)-1-methyl-1H-indazol-3-yl)piperidine-2,6-dione C138;
3-(7-((1-(3-(tert-butyl) bicyclo[1.1.1]pentane-1-carbonyl)piperidin-4-yl)oxy)-1-methyl-1H-indazol-3-yl)piperidine-2,6-dione C139;
3-(1-methyl-7-((1-(tetrahydro-2H-thiopyran-4-carbonyl)piperidin-4-yl)oxy)-1H-indazol-3-yl)piperidine-2,6-dione C140;
3-(1-methyl-7-((1-(1-methyl-5-(trifluoromethyl)-1H-imidazole-4-carbonyl)-piperidin-4-yl)oxy)-1H-indazol-3-yl)piperidine-2,6-dione C141;
3-(7-((1-(2-(benzyloxy)acetyl)piperidin-4-yl)oxy)-1-methyl-1H-indazol-3-yl)-piperidine-2,6-dione C142;
3-(7-((1-(1-isopropyl-1H-pyrazole-5-carbonyl)piperidin-4-yl)oxy)-1-methyl-1H-indazol-3-yl)piperidine-2,6-dione C143;
3-(1-methyl-7-((1-(3-(trifluoromethoxy)benzoyl)piperidin-4-yl)oxy)-1H-indazol-3-yl)piperidine-2,6-dione C144;
3-(1-methyl-7-((1-(2-(thiophen-2-yl)acetyl)piperidin-4-yl)oxy)-1H-indazol-3-yl)piperidine-2,6-dione C145;
3-(1-methyl-7-((1-(2-methyl-2,4,5,6-tetrahydrocyclopenta[c]pyrazole-3-carbonyl)piperidin-4-yl)oxy)-1H-indazol-3-yl)piperidine-2,6-dione C146;
3-(1-methyl-7-((1-(thiophene-2-carbonyl)piperidin-4-yl)oxy)-1H-indazol-3-yl)-piperidine-2,6-dione C147;
4-(4-((3-(2,6-dioxopiperidin-3-yl)-1-methyl-1H-indazol-7-yl)oxy) piperidine-1-carbonyl)benzonitrile C148;
3-(7-((1-(cyclopentanecarbonyl)piperidin-4-yl)oxy)-1-methyl-1H-indazol-3-yl)-piperidine-2,6-dione C149;
3-(7-((1-(2-(dimethylamino)pyrimidine-4-carbonyl)piperidin-4-yl)oxy)-1-methyl-1H-indazol-3-yl)piperidine-2,6-dione C150;
3-(7-((1-(1-(tert-butyl)-1H-pyrazole-3-carbonyl)piperidin-4-yl)oxy)-1-methyl-1H-indazol-3-yl)piperidine-2,6-dione C151;
(E)-3-(7-((1-cinnamoylpiperidin-4-yl)oxy)-1-methyl-1H-indazol-3-yl)piperidine-2,6-dione C152;
5-(2-(4-((3-(2,6-dioxopiperidin-3-yl)-1-methyl-1H-indazol-7-yl)oxy) piperidin-1-yl)-2-oxoethyl)-1-methyl-1H-pyrrole-2-carbonitrile C153;
3-(1-methyl-7-((1-(thiazole-4-carbonyl)piperidin-4-yl)oxy)-1H-indazol-3-yl)-piperidine-2,6-dione C154;
3-(1-methyl-7-((1-(thiophene-3-carbonyl)piperidin-4-yl)oxy)-1H-indazol-3-yl)-piperidine-2,6-dione C155;
3-(7-((1-(1-acetylpiperidine-4-carbonyl)piperidin-4-yl)oxy)-1-methyl-1H-indazol-3-yl)piperidine-2,6-dione C156;
3-(7-((1-(3-isopropoxybenzoyl)piperidin-4-yl)oxy)-1-methyl-1H-indazol-3-yl)-piperidine-2,6-dione C157;
3-(1-methyl-7-((1-(pyrimidine-4-carbonyl)piperidin-4-yl)oxy)-1H-indazol-3-yl)-piperidine-2,6-dione C158;
3-(1-methyl-7-((1-(5-phenyloxazole-4-carbonyl)piperidin-4-yl)oxy)-1H-indazol-3-yl)piperidine-2,6-dione C159;
3-(7-((1-(2,4-dimethyloxazole-5-carbonyl)piperidin-4-yl)oxy)-1-methyl-1H-indazol-3-yl)piperidine-2,6-dione C160;
3-(1-methyl-7-((1-(2-(phenylthio) acetyl)piperidin-4-yl)oxy)-1H-indazol-3-yl)-piperidine-2,6-dione C161;
3-(1-methyl-7-((1-(2-(1-methylpiperidin-4-yl)acetyl)piperidin-4-yl)oxy)-1H-indazol-3-yl)piperidine-2,6-dione C162;
6-(4-((3-(2,6-dioxopiperidin-3-yl)-1-methyl-1H-indazol-7-yl)oxy) piperidine-1-carbonyl) nicotinonitrile C163; or
3-(7-((1-(cyclobutanecarbonyl)piperidin-4-yl)oxy)-1-methyl-1H-indazol-3-yl)-piperidine-2,6-dione C164;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is a compound of:
N-(3-cyclopropyl-1H-pyrazol-5-yl)-2-(3-(2,6-dioxopiperidin-3-yl)-1H-indazol-1-yl)acetamide D001;
2-(3-(2,6-dioxopiperidin-3-yl)-1H-indazol-1-yl)-N-(4-(methylsulfonyl)phenyl)-acetamide D002;
N-(4-(1H-imidazol-5-yl)phenyl)-2-(3-(2,6-dioxopiperidin-3-yl)-1H-indazol-1-yl)-acetamide D003;
2-(3-(2,6-dioxopiperidin-3-yl)-1H-indazol-1-yl)-N-(1-(tetrahydro-2H-pyran-4-yl)-1H-pyrazol-4-yl)acetamide D004;
2-(3-(2,6-dioxopiperidin-3-yl)-1H-indazol-1-yl)-N-(3-ethylisoxazol-5-yl)-acetamide D005;
2-(3-(2,6-dioxopiperidin-3-yl)-1H-indazol-1-yl)-N-(1-methyl-3-(trifluoromethyl)-1H-pyrazol-4-yl)acetamide D006;
2-(3-(2,6-dioxopiperidin-3-yl)-1H-indazol-1-yl)-N-(thiazol-4-yl)acetamide D007; 2-(3-(2,6-dioxopiperidin-3-yl)-1H-indazol-1-yl)-N-(4-(N-methylsulfamoyl)-phenyl)acetamide D008;
2-(3-(2,6-dioxopiperidin-3-yl)-1H-indazol-1-yl)-N-((1-hydroxycyclopropyl)-methyl)acetamide D009;
2-(3-(2,6-dioxopiperidin-3-yl)-1H-indazol-1-yl)-N-(2-methylthiazol-5-yl)-acetamide D010;
2-(3-(2,6-dioxopiperidin-3-yl)-1H-indazol-1-yl)-N-(3-methylisothiazol-5-yl)-acetamide DO11; 2-(3-(2,6-dioxopiperidin-3-yl)-1H-indazol-1-yl)-N-(pyridazin-3-yl)acetamide D012;
N-(3-cyclopropyl-1-methyl-1H-pyrazol-5-yl)-2-(3-(2,6-dioxopiperidin-3-yl)-1H-indazol-1-yl)acetamide D013;
N-(3,5-dimethylisoxazol-4-yl)-2-(3-(2,6-dioxopiperidin-3-yl)-1H-indazol-1-yl)-acetamide DO14;
2-(3-(2,6-dioxopiperidin-3-yl)-1H-indazol-1-yl)-N-(1-isopropyl-1H-pyrazol-4-yl)-acetamide D015;
N-(3-(tert-butyl)-1-phenyl-1H-pyrazol-5-yl)-2-(3-(2,6-dioxopiperidin-3-yl)-1H-indazol-1-yl)acetamide D016;
2-(3-(2,6-dioxopiperidin-3-yl)-1H-indazol-1-yl)-N-(3-methylisoxazol-5-yl)-acetamide D017;
2-(3-(2,6-dioxopiperidin-3-yl)-1H-indazol-1-yl)-N-(4-(oxazol-5-yl)phenyl)-acetamide D018;
N-(1-(tert-butyl)-3-methyl-1H-pyrazol-5-yl)-2-(3-(2,6-dioxopiperidin-3-yl)-1H-indazol-1-yl)acetamide D019;
N-(3,5-dimethyl-1H-pyrazol-4-yl)-2-(3-(2,6-dioxopiperidin-3-yl)-1H-indazol-1-yl)acetamide D020;
2-(3-(2,6-dioxopiperidin-3-yl)-1H-indazol-1-yl)-N-(3-methylisothiazol-4-yl)-acetamide DO21;
2-(3-(2,6-dioxopiperidin-3-yl)-1H-indazol-1-yl)-N-(4-(2-methyl-1H-imidazol-1-yl)phenyl)acetamide D022;
2-(3-(2,6-dioxopiperidin-3-yl)-1H-indazol-1-yl)-N-(4-(4-methylpiperazin-1-yl)-phenyl)acetamide D023;
2-(3-(2,6-dioxopiperidin-3-yl)-1H-indazol-1-yl)-N-(1-methyl-1H-pyrazol-5-yl)-acetamide D024;
N-(4-(1H-imidazol-2-yl)phenyl)-2-(3-(2,6-dioxopiperidin-3-yl)-1H-indazol-1-yl)-acetamide D025;
N-(3-(1H-imidazol-1-yl)phenyl)-2-(3-(2,6-dioxopiperidin-3-yl)-1H-indazol-1-yl)-acetamide D026;
2-(3-(2,6-dioxopiperidin-3-yl)-1H-indazol-1-yl)-N-((1-methyl-1H-pyrazol-3-yl)-methyl)acetamide D027;
2-(3-(2,6-dioxopiperidin-3-yl)-1H-indazol-1-yl)-N-((1-methyl-1H-imidazol-5-yl)-methyl)acetamide D028;
2-(3-(2,6-dioxopiperidin-3-yl)-1H-indazol-1-yl)-N-(1-methylpiperidin-4-yl)-acetamide D029;
2-(3-(2,6-dioxopiperidin-3-yl)-1H-indazol-1-yl)-N-(3-hydroxycyclopentyl)-acetamide DO30;
N-(1,1-dioxidothietan-3-yl)-2-(3-(2,6-dioxopiperidin-3-yl)-1H-indazol-1-yl)-acetamide D031;
N-(3-(tert-butyl)phenyl)-2-(3-(2,6-dioxopiperidin-3-yl)-1H-indazol-1-yl)-acetamide D032;
N-(2-(benzyloxy)ethyl)-2-(3-(2,6-dioxopiperidin-3-yl)-1H-indazol-1-yl)-acetamide D033;
2-(3-(2,6-dioxopiperidin-3-yl)-1H-indazol-1-yl)-N-(3-(oxazol-5-yl)phenyl)-acetamide D034;
2-(3-(2,6-dioxopiperidin-3-yl)-1H-indazol-1-yl)-N-(3-isopropoxyphenyl)-acetamide D035;
2-(3-(2,6-dioxopiperidin-3-yl)-1H-indazol-1-yl)-N-(4-(trifluoromethoxy)phenyl)-acetamide D036;
2-(3-(2,6-dioxopiperidin-3-yl)-1H-indazol-1-yl)-N-(4-isopropylbenzyl)acetamide D037;
2-(3-(2,6-dioxopiperidin-3-yl)-1H-indazol-1-yl)-N-(4-hydroxybenzyl)acetamide D038;
2-(3-(2,6-dioxopiperidin-3-yl)-1H-indazol-1-yl)-N-(4-methyltetrahydro-2H-pyran-4-yl)acetamide D039;
N-cyclohexyl-2-(3-(2,6-dioxopiperidin-3-yl)-1H-indazol-1-yl)acetamide D040;
2-(3-(2,6-dioxopiperidin-3-yl)-1H-indazol-1-yl)-N-(thiophen-2-ylmethyl)-acetamide D041;
2-(3-(2,6-dioxopiperidin-3-yl)-1H-indazol-1-yl)-N-((3-hydroxycyclopentyl)-methyl)acetamide D042;
2-(3-(2,6-dioxopiperidin-3-yl)-1H-indazol-1-yl)-N-(tetrahydro-2H-pyran-4-yl)-acetamide D043;
N-(2-(cyclopentyloxy)ethyl)-2-(3-(2,6-dioxopiperidin-3-yl)-1H-indazol-1-yl)-acetamide D044;
2-(3-(2,6-dioxopiperidin-3-yl)-1H-indazol-1-yl)-N-(3-fluorobicyclo[1.1.1]pentan-1-yl)acetamide D045;
2-(3-(2,6-dioxopiperidin-3-yl)-1H-indazol-1-yl)-N-(4-(p-tolyloxy)phenyl)-acetamide D046;
2-(3-(2,6-dioxopiperidin-3-yl)-1H-indazol-1-yl)-N-(thiazol-2-ylmethyl)acetamide D047;
N-(cyclopropylmethyl)-2-(3-(2,6-dioxopiperidin-3-yl)-1H-indazol-1-yl)-acetamide D048;
N-cyclopropyl-2-(3-(2,6-dioxopiperidin-3-yl)-1H-indazol-1-yl)acetamide D049;
2-(3-(2,6-dioxopiperidin-3-yl)-1H-indazol-1-yl)-N-(1-methylazetidin-3-yl)-acetamide D050;
N-(cyclopentylmethyl)-2-(3-(2,6-dioxopiperidin-3-yl)-1H-indazol-1-yl)acetamide D051;
2-(3-(2,6-dioxopiperidin-3-yl)-1H-indazol-1-yl)-N-neopentylacetamide D052;
2-(3-(2,6-dioxopiperidin-3-yl)-1H-indazol-1-yl)-N-(tetrahydrofuran-3-yl)-acetamide D053;
N-cyclopentyl-2-(3-(2,6-dioxopiperidin-3-yl)-1H-indazol-1-yl)acetamide D054; or 2-(3-(2,6-dioxopiperidin-3-yl)-1H-indazol-1-yl)-N-(2-(methylamino)-2-oxo-ethyl)acetamide D055;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, a compound provided herein is a molecular glue. In certain embodiments, a compound provided herein is monovalent.

In certain embodiments, a compound provided herein is a compound provided herein has a molecular weight of no greater than about 750 Da, no greater than about 700 Da, no greater than about 650 Da, no greater than about 600 Da, no greater than about 550 Da, or no greater than about 500 Da. In certain embodiments, a compound provided herein is a compound provided herein has a molecular weight of no greater than about 750 Da. In certain embodiments, a compound provided herein is a compound provided herein has a molecular weight of no greater than about 700 Da. In certain embodiments, a compound provided herein is a compound provided herein has a molecular weight of no greater than about 650 Da. In certain embodiments, a compound provided herein is a compound provided herein has a molecular weight of no greater than about 600 Da. In certain embodiments, a compound provided herein is a compound provided herein has a molecular weight of no greater than about 550 Da. In certain embodiments, a compound provided herein is a compound provided herein has a molecular weight of no greater than about 500 Da.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; 170 or 180 for oxygen, and ³⁴S, ³⁵S, or ³⁶S for sulfur.

In certain embodiments, a compound provided herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, a compound provided herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of a compound provided herein, as specified as deuterium-enriched, has deuterium enrichment of no less than about 50%, no less than about 70%, no less than about 80%, no less than about 90%, or no less than about 98%.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers unless a particular stereochemistry is specified. Where a compound provided herein contains an alkenyl group, the compound may exist as one or mixture of geometric cisltrans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its(S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, provided herein is a pharmaceutical composition, comprising a compound provided herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

The pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, FL, 2008.

In one embodiment, the pharmaceutical composition provided herein is formulated in a dosage form for oral administration. In another embodiment, the pharmaceutical composition provided herein is formulated in a dosage form for parenteral administration. In yet another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for topical administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s)(e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### Methods of Use

In one embodiment, provided herein is a method of treating, preventing, or ameliorating one or more symptoms of a disorder, disease, or condition mediated by a zinc-figure protein, CK1α, GSPT1, and/or PDE6D in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the disorder, disease, or condition mediated by a zinc-figure protein, CK1α, GSPT1, and/or PDE6D is a proliferative disease.

In certain embodiments, the zinc-figure protein is Aiolos, Helios, or Ikaros. In certain embodiments, the zinc-figure protein is Aiolos. In certain embodiments, the zinc-figure protein is Helios. In certain embodiments, the zinc-figure protein is Ikaros.

In another embodiment, provided herein is a method of treating, preventing, or ameliorating one or more symptoms of a proliferative disease in a subject, comprising administering to the subject in need thereof a therapeutically effective amount of a compound provided herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the proliferative disease is cancer. In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is unresectable. In certain embodiments, the cancer is metastatic.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In certain embodiments, the therapeutically effective amount of a compound provided herein is ranging from about 0.1 to about 100 mg/kg/day, from about 0.1 to about 50 mg/kg/day, from about 0.1 to about 60 mg/kg/day, from about 0.1 to about 50 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount of a compound provided herein is ranging from about 0.1 to about 100 mg/kg/day. In another embodiment, the therapeutically effective amount of a compound provided herein is ranging from about 0.1 to about 50 mg/kg/day. In yet another embodiment, the therapeutically effective amount of a compound provided herein is ranging from about 0.1 to about 60 mg/kg/day. In yet another embodiment, the therapeutically effective amount of a compound provided herein is ranging from about 0.1 to about 50 mg/kg/day. In yet another embodiment, the therapeutically effective amount of a compound provided herein is ranging from about 0.1 to about 25 mg/kg/day. In yet another embodiment, the therapeutically effective amount of a compound provided herein is ranging from about 0.1 to about 20 mg/kg/day. In yet another embodiment, the therapeutically effective amount of a compound provided herein is ranging from about 0.1 to about 15 mg/kg/day. In yet another embodiment, the therapeutically effective amount of a compound provided herein is ranging from about 0.1 to about 10 mg/kg/day. In still another embodiment, the therapeutically effective amount of a compound provided herein is ranging from about 0.1 to about 5 mg/kg/day.

It is understood that the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both. For example, a dose of 1 mg/m²/day for a 65 kg human is approximately equal to 58 mg/kg/day.

Depending on the disorder, disease, or condition to be treated and the subject's condition, a compound provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A compound provided herein may be formulated in suitable dosage unit with a pharmaceutically acceptable excipient, carrier, adjuvant, or vehicle, appropriate for each route of administration.

In one embodiment, a compound provided herein is administered orally. In another embodiment, a compound provided herein is administered parenterally. In yet another embodiment, a compound provided herein is administered intravenously. In yet another embodiment, a compound provided herein is administered intramuscularly. In yet another embodiment, a compound provided herein is administered subcutaneously. In still another embodiment, a compound provided herein is administered topically.

A compound provided herein can be delivered as a single dose such as, e.g., a single bolus injection, or oral tablets or pills; or over time such as, e.g., continuous infusion over time or divided bolus doses over time. A compound provided herein can be administered repetitively, if necessary, for example, until the subject experiences stable disease or regression, or until the subject experiences disease progression or unacceptable toxicity.

A compound provided herein can be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days.

In certain embodiments, a compound provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered orally. In another embodiment, a compound provided herein is administered intravenously. Thus, in accordance with these embodiments, a compound provided herein is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration, orally or by IV. In another embodiment, a compound provided herein is administered by one mode of administration, e.g., by IV, whereas the second agent (an anticancer agent) is administered by another mode of administration, e.g., orally.

In one embodiment, provided herein is a method of inhibiting the growth of a cell, comprising contacting the cell with an effective amount of a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a cancerous cell. In certain embodiments, the cell is a human cell. In certain embodiments, the cell is a human cancerous cell.

In one embodiment, provided herein is inducing degradation of a protein, comprising contacting the protein with an effective amount of a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the protein is a zinc-figure protein, CK1α, GSPT1, or PDE6D. In certain embodiments, the protein is a zinc-figure protein. In certain embodiments, the protein is CK1α. In certain embodiments, the protein is GSPT1. In certain embodiments, the protein is PDE6D.

In certain embodiments, the zinc-figure protein is Aiolos, Helios, or Ikaros. In certain embodiments, the zinc-figure protein is Aiolos. In certain embodiments, the zinc-figure protein is Helios. In certain embodiments, the zinc-figure protein is Ikaros.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); ACN (acetonitrile); Bn (benzyl); Boc (tert-butoxycarbonyl); DCM (dichloro-methane); DIAD (diisopropyl azodicarboxylate); DIPEA (N,N-diisopropylethylamine); DMF (dimethylformamide); DMAP (4-dimethylamino-pyridine); DMSO (dimethyl sulfoxide); EtOAc (ethyl acetate); HATU (hexafluorophosphate azabenzotriazole tetramethyl uronium); MeOH (methanol); NMI (N-methylimidazole); NMP (N-methyl-2-pyrrolidone); Pd (dppf) Cl2 ([1,1′-bis(diphenylphosphino) ferrocene]dichloro-palladium (II)); PE (petroleum ether); tBu (tert-butyl); T₄P (butylphosphonic anhydride); TCFH (chloro-N,N,N,N-tetramethylformamidinium hexa-fluorophosphate); TEA (triethylamine); TFA (trifluoroacetic acid); THF (tetrahydrofuran); TMSOK (potassium trimethylsilanolate); TPP (triphenylphosphate); LCMS (liquid chromatography-mass spectrometry); MS (mass spectrometry); NMR (nuclear magnetic resonance); and prep-HPLC (preparative high performance liquid chromatography).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of N-(2-cyanoethyl)-2-((3-(2,6-dioxopiperidin-3-yl)-1-methyl-1H-indazol-6-yl)-oxy)acetamide A012

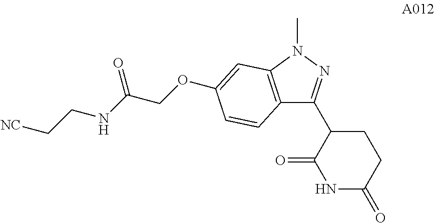 [SMILES: Cn1nc(C2CCC(=O)NC2=O)c2ccc(OCC(=O)NCCC#N)cc21]

Compound A012 was prepared as shown in Scheme 1.

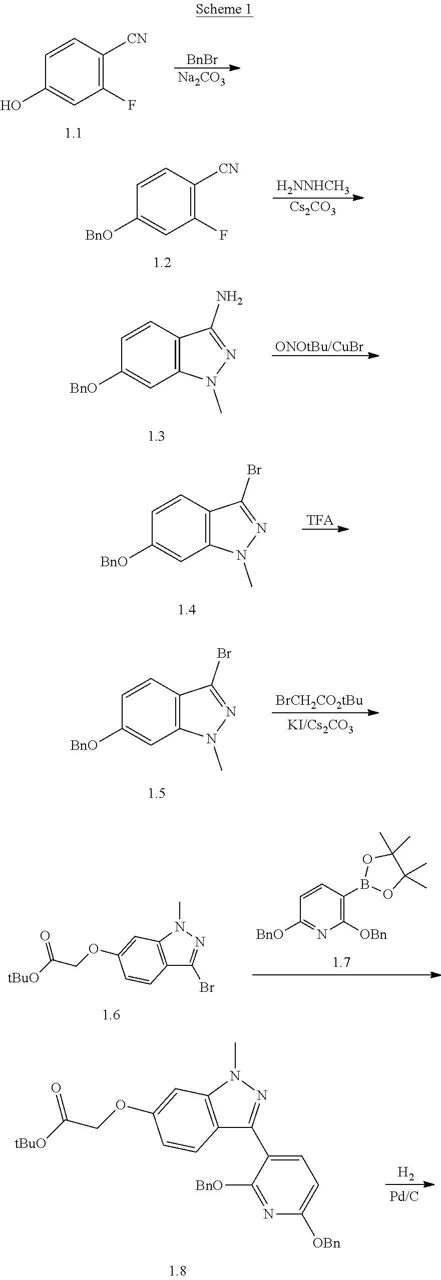

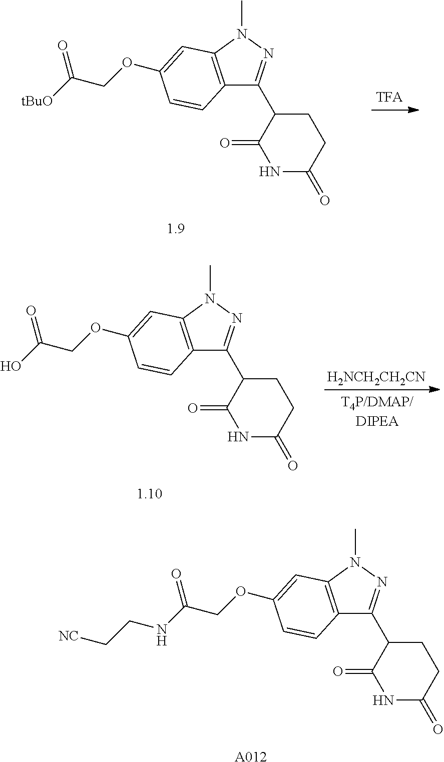

Preparation of 4-(benzyloxy)-2-fluorobenzonitrile. To a solution of 2-fluoro-4-hydroxybenzonitrile 1.1 (50 g, 364 mmol) in DMF (60 mL) were added BnBr (52 mL, 436 mmol) and Na₂CO₃ (77.2 g, 728 mmol). After stirred overnight, the reaction mixture was diluted with water (100 mL) and extracted with EtOAc (300 mL×2). The combined organic layers were dried over anhydrous Na₂SO₄, filtered, and concentrated under reduced pressure to yield a crude product, which was purified by silica-gel column chromatography eluting with EtOAc/PE to afford compound 1.2 (79 g) in 96% yield. MS (ESI) m z: 226.2 [M−1]⁻.

Preparation of 6-(benzyloxy)-1-methyl-1H-indazol-3-amine 1.3. To a solution of compound 1.2 (73 g, 322 mmol) in DMF (500 mL) were added methylhydrazine (74 g, 168 mmol) and Cs₂CO₃ (210 g, 644 mmol). After heated in a high pressure reactor at 120° C. overnight, the reaction mixture was diluted with water (200 mL) and extracted with EtOAc (300 mL×2). The combined organic layers were dried over anhydrous Na₂SO₄, filtered, and concentrated under reduced pressure to yield a crude product, which was purified by silica-gel column chromatography eluting with EtOAc/PE to afford compound 1.3 (42.7 g) in 52% yield. MS (ESI) m z: 254.1 [M+1]⁺.

Preparation of 6-(benzyloxy)-3-bromo-1-methyl-1H-indazole 1.4. To a solution of compound 1.3 (12.3 g, 48.3 mmol) in ACN (120 mL) were added tert-butyl nitrite (8.62 mL, 72.5 mmol) and CuBr (9.05 g, 63 mmol). After stirred overnight, the reaction mixture was concentrated under reduced pressure to yield a crude product, which was purified by silica-gel column chromatography eluting with EtOAc/PE to afford compound 1.4 (5.1 g) in 33% yield. MS (ESI) m z: 317.1, 319.1 [M+1, M+3]⁺.

Preparation of 3-bromo-1-methyl-1H-indazol-6-ol 1.5. A solution of compound 1.4 (4.9 g, 15.5 mmol) in TFA (150 mL) was stirred at 80° C. for 4 h. The reaction mixture was concentrated under reduced pressure to yield a crude product, which was purified by silica-gel column chromatography eluting with EtOAc/PE to afford compound 1.5 (3.1 g) in 86% yield. MS (ESI) m z: 227.0, 229.0 [M+1, M+3]⁺.

Preparation of tert-butyl 2-((3-bromo-1-methyl-1H-indazol-6-yl)oxy)acetate 1.6. To a solution of compound 1.5 (10.2 g, 44.8 mmol) in DMF (100 mL) were added Cs₂CO₃ (21.8 g, 67 mmol), KI (3.7 g, 22.4 mmol), and tert-butyl 2-bromoacetate (8.7 g, 44.8 mmol). After stirred overnight, the reaction mixture was diluted with water (100 mL) and extracted with EtOAc (150 mL×2). The combined organic layers were concentrated under reduced pressure to yield a crude product, which was purified by silica-gel column chromatography eluting with EtO Ac/PE to afford compound 1.6 (15 g) in 93% yield. MS (ESI) m z: 341.1, 343.1 [M+1, M+3]⁺.

Preparation of tert-butyl 2-((3-(2,6-bis(benzyloxy)pyridin-3-yl)-1-methyl-1H-indazol-6-yl)oxy)acetate 1.8. To a solution of compound 1.6 (4.27 g, 12.5 mmol) and 2,6-bis(benzyloxy)-3-(4,4,5,5-tetramethyl-1,3,2-dioxaborolan-2-yl)pyridine 1.7 (10.4 g, 25 mmol) in 1,4-dioxane (150 mL) and water (25 mL) were added K₂CO₃ (3.45 g, 25 mmol) and Pd (dppf) Cl₂ (915 mg, 1.25 mmol) under N₂. After heated at 85° C. under N₂ overnight, the reaction mixture was diluted with water (40 mL) and extracted with EtOAc (100 mL×3). The combined organic layers were dried over anhydrous Na₂SO₄, filtered, and concentrated under reduced pressure to yield a crude product, which was purified by silica-gel column chromatography eluting with EtO Ac/PE to afford compound 1.8 (5.26 g) in 76% yield. MS (ESI) m z: 552.3 [M+1]⁺.

Preparation of ter-butyl 2-((3-(2,6-dioxopiperidin-3-yl)-1-methyl-1H-indazol-6-yl)oxy)acetate 1.9. To a solution of compound 1.8 (4.1 g, 7.43 mmol) in MeOH (40 mL) and THF (40 mL) were added Pd/C (1 g). After stirred under H₂ overnight, the reaction mixture was filtered and concentrated under reduced pressure to afford compound 1.9 (2.62 g) in 95% yield. MS (ESI) m z: 374.2 [M+1]⁺.

Preparation of 2-((3-(2,6-dioxopiperidin-3-yl)-1-methyl-1H-indazol-6-yl)oxy)-acetic acid 1.10. To a solution of compound 1.9 (240 mg, 0.64 mmol) in DCM (6 mL) was added TFA (6 mL). After stirred overnight, the reaction mixture was concentrated under reduced pressure to yield a crude product, which was purified by reverse-phase prep-HPLC to afford compound 1.10 (134 mg) in 64% yield. ¹H NMR (400 MHZ, DMSO-d₆) δ 10.86 (s, 1H), 7.58 (d, J=9.2 Hz, 1H), 7.06 (d, J=2 Hz, 1H), 6.78 (dd, J=2.0, 8.8 Hz, 1H), 4.77 (s, 2H), 4.31 (dd, J=4.8, 9.2 Hz, 1H), 3.92 (s, ³H), 2.65-2.51 (m, 2H), 2.50-2.32 (m, 1H), 2.31-2.15 (m, 1H); MS (ESI) m z: 318.1 [M+1]⁺.

Preparation of N-(2-cyanoethyl)-2-((3-(2,6-dioxopiperidin-3-yl)-1-methyl-1H-indazol-6-yl)-oxy)acetamide A012. To a solution of compound 1.10 (47.6 mg, 0.15 mmol) and 3-aminopropanenitrile (12.6 mg, 0.18 mmol) in DCM (1.5 mL) were added DIPEA (0.208 mL, 1.5 mmol), DMAP (18.3 mg, 0.15 mmol), and 50% T₄P in EtOAc (0.180 mL). After stirred at 50° C. for 16 h, the reaction mixture was concentrated under reduced pressure to yield a crude residue, which was purified by reverse-phase prep-HPLC to afford compound A012 (28.5 mg). MS (ESI) m/z: 370.2 [M+1]⁺.

### Example 2

### Preparation of 2-((3-(2,6-dioxopiperidin-3-yl)-1-methyl-1H-indazol-6-yl)oxy)-N-((4-hydroxy-tetrahydro-2H-pyran-4-yl)methyl)acetamide A027

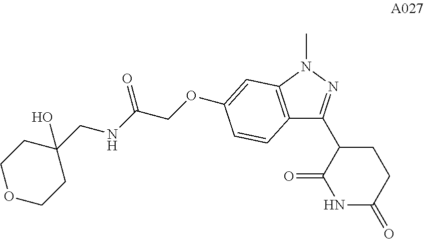 [SMILES: Cn1nc(C2CCC(=O)NC2=O)c2ccc(OCC(=O)NCC3(O)CCOCC3)cc21]

Compound A027 was prepared as shown in Scheme 2.

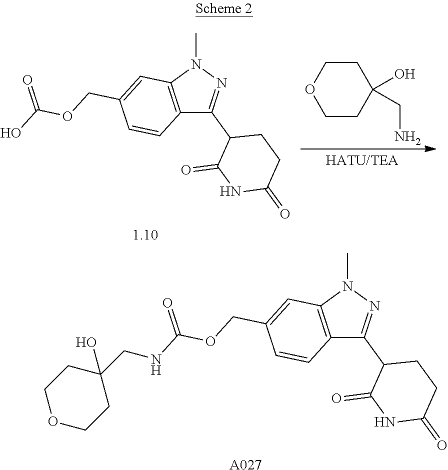 [SMILES: Cn1nc(C2CCC(=O)NC2=O)c2ccc(COC(=O)O)cc21 ; NCC1(O)CCOCC1 ; Cn1nc(C2CCC(=O)NC2=O)c2ccc(COC(=O)NCC3(O)CCOCC3)cc21 ; "A027"]

To a solution of 2-((3-(2,6-dioxopiperidin-3-yl)-1-methyl-1H-indazol-6-yl)oxy)acetic acid 1.10 (47.6 mg, 0.15 mmol) and 4-(aminomethyl)tetrahydro-2H-pyran-4-ol (23.6 mg, 0.18 mmol) in DMF (1.5 mL) were added TEA (0.208 mL, 1.5 mmol) and HATU (87.4 mg, 0.23 mmol). After stirred at 50° C. for 16 h, the reaction mixture was purified by reverse-phase prep-HPLC to afford compound A027 (31 mg). MS (ESI) m/z: 431.2 [M+1]⁺.

### Example 3

### Preparation of N-cyclopropyl-2-((3-(2,6-dioxopiperidin-3-yl)-1-methyl-1H-indazol-6-yl)oxy)-acetamide A070

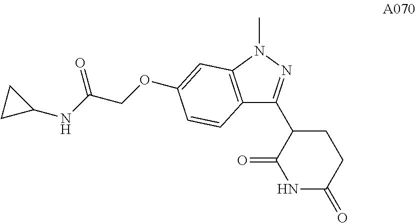 [SMILES: Cn1nc(C2CCC(=O)NC2=O)c2ccc(OCC(=O)NC3CC3)cc21]

Compound A070 was prepared as shown in Scheme 3.

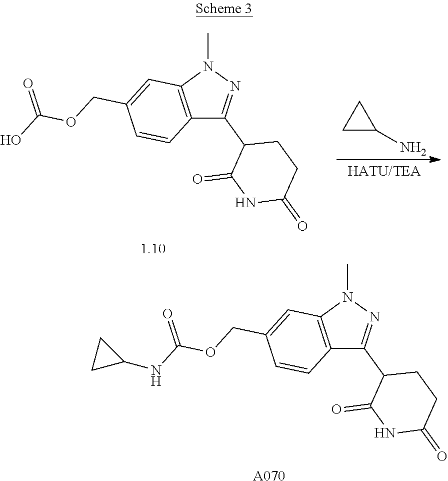

To a solution of 2-((3-(2,6-dioxopiperidin-3-yl)-1-methyl-1H-indazol-6-yl)oxy)acetic acid 1.10 (47.6 mg, 0.15 mmol) and cyclopropanamine (10.3 mg, 0.18 mmol) in DMF (1.5 mL) were added TEA (0.208 mL, 1.5 mmol) and HATU (87.4 mg, 0.23 mmol). After stirred at 30° C. for 16 h, the reaction mixture was purified by reverse-phase prep-HPLC to afford compound A070 (23 mg). MS (ESI) m/z: 357.2 [M+1]⁺.

### Example 4

### Preparation of N-(3-cyano-4,5-dimethylthiophen-2-yl)-2-((3-(2,6-dioxopiperidin-3-yl)-1-methyl-1H-indazol-7-yl)oxy)acetamide B042

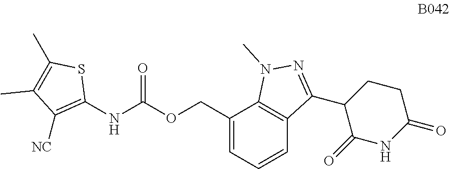 [SMILES: Cc1sc(NC(=O)OCc2cccc3c(C4CCC(=O)NC4=O)nn(C)c23)c(C#N)c1C]

Compound B042 was prepared as shown in Scheme 4.

Preparation of 1-(benzyloxy)-3-bromo-2-fluorobenzene 4.2. To a solution of 3-bromo-2-fluorophenol 4.1 (20 g, 104 mmol) in DMF (70 mL) were added BnBr (15 g, 125 mmol) and Na₂CO₃ (22 g, 208 mmol). After stirred overnight, the reaction mixture was diluted with water (100 mL) and extracted with EtOAc (150 mL×2). The combined organic layers were concentrated under reduced pressure to give a crude product, which was purified by silica-gel column chromatography eluting with EtOAc/PE to afford compound 4.2 (28 g) in 96% yield. MS (ESI) m/z: 282.3 [M+1]⁺.

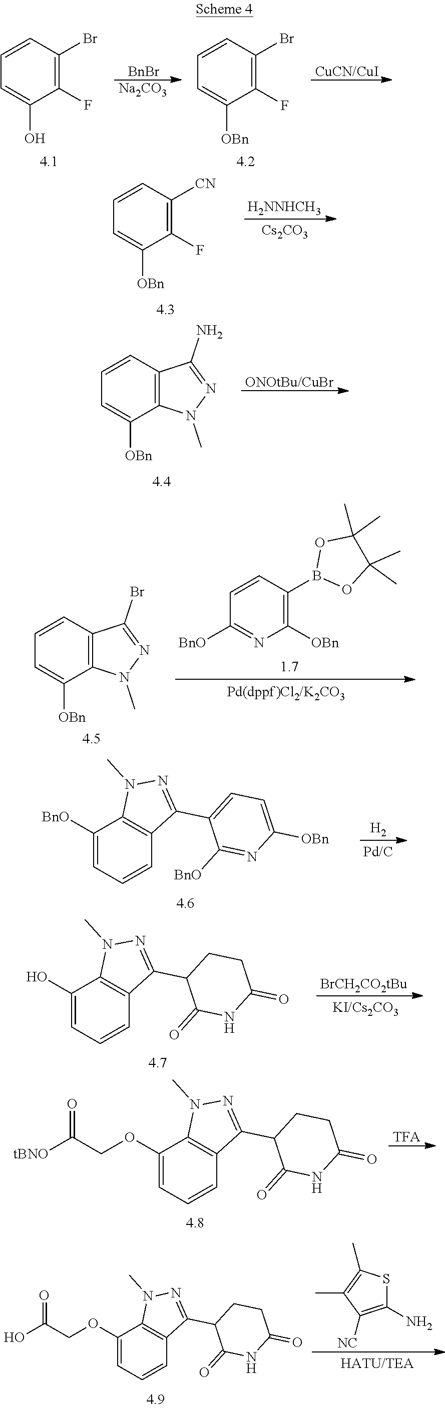

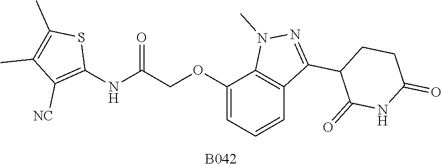

Preparation of 3-(benzyloxy)-2-fluorobenzonitrile 4.3. To a solution of compound 4.2 (28 g, 100 mmol) in NMP (100 mL) were added CuCN (13.4 g, 150 mmol) and CuI (5.7 g, 30 mmol). After stirred at ¹⁸O ° C. for 4 h, the reaction mixture was diluted with water (100 mL) and extracted with EtOAc (150 mL×2). The combined organic layers were dried over anhydrous Na₂SO₄, filtered, and concentrated under reduced pressure to give a crude product, which was purified by silica-gel column chromatography eluting with EtOAc/PE to afford compound 4.3 (18.2 g) in 80% yield. MS (ESI) m z: 228.1 [M+1]⁺.

Preparation of 7-(benzyloxy)-1-methyl-1H-indazol-3-amine 4.4. To a solution of compound 4.3 (18.2 g, 80 mmol) in DMF (200 mL) were added methylhydrazine (18.4 g, 400 mmol) and Cs₂CO₃ (52.1 g, 160 mmol). After heated in a high pressure reactor at 120° C. overnight, the reaction mixture was diluted with water (100 mL) and extracted with EtOAc (200 mL×2). The combined organic layers were concentrated under reduced pressure to yield a crude product, which was purified by silica-gel column chromatography eluting with EtOAc/PE to afford compound 4.4 (9.6 g) in 47% yield. MS (ESI) m z: 254.2 [M+1]⁺.

Preparation of 6-(benzyloxy)-3-bromo-1-methyl-1H-indazole 4.5. To a solution of compound 4.4 (5 g, 19.7 mmol) in ACN (30 mL) were added tert-butyl nitrite (3.5 mL, 29.6 mmol) and CuBr (3.7 g, 25.6 mmol). After stirred overnight, the reaction mixture was concentrated under reduced pressure to yield a crude product, which was purified by silica-gel column chromatography eluting with EtOAc/PE to afford compound 4.5 (1.8 g) in 21% yield. MS (ESI) m z: 317.1 [M+1]⁺.

Preparation of 7-(benzyloxy)-3-(2,6-bis(benzyloxy)pyridin-3-yl)-1-methyl-1H-indazole 4.6. To a solution of compound 4.5 (500 mg, 1.6 mmol) and 2,6-bis(benzyloxy)-3-(4,4,5,5-tetramethyl-1,3,2-dioxaborolan-2-yl)pyridine 1.7 (1 g, 2.4 mmol) in 1,4-dioxane/water (6:1, 30 mL) were added K₂CO₃ (440 mg, 8 mmol) and Pd (dppf) Cl₂ (115 mg, 0.16 mmol) under N₂. After heated at 100° C. under N₂ overnight, the reaction mixture was diluted with water (20 mL) and extracted with EtOAc (30 mL×3). The combined organic layers were dried over anhydrous Na₂SO₄, filtered, and concentrated under reduced pressure to give a crude product, which was purified by silica-gel column chromatography eluting with EtOAc/PE to afford compound 4.6 (800 mg) in 89% yield. MS (ESI) m z: 528.3 [M+1]⁺.

Preparation of 3-(7-hydroxy-1-methyl-1H-indazol-3-yl)-piperidine-2,6-dione 4.7. To a solution of compound 4.6 (500 mg, 0.95 mmol) in MeOH (10 mL) and DCM (10 mL) was added 20% Pd/C (200 mg). After stirred under H₂ overnight, the reaction mixture was filtered and concentrated under reduced pressure to afford compound 4.7 (200 mg) in 82% yield.

Preparation of tert-butyl 2-((3-(2,6-dioxopiperidin-3-yl)-1-methyl-1H-indazol-7-yl)oxy)acetate 4.8. To a solution of compound 4.7 (200 mg, 0.77 mmol) in DMF (5 mL) were added K₂CO₃ (128 mg, 0.92 mmol), KI (40 mg, 0.24 mmol), and tert-butyl 2-bromoacetate (120 mg, 0.6 mmol). After stirred overnight, the reaction mixture was diluted with water (10 mL) and extracted with EtOAc (20 mL×2). The combined organic layers were concentrated under reduced pressure to give a crude product, which was purified by silica-gel column chromatography eluting with EtOAc/PE to afford compound 4.8 (150 mg) in 56% yield. MS (ESI) m z: 374.2 [M+1]⁺.

Preparation of 2-((3-(2,6-dioxopiperidin-3-yl)-1-methyl-1H-indazol-7-yl)oxy)-acetic acid 4.9. To a solution of compound 4.8 (100 mg, 0.27 mmol) in DCM (3 mL) was added TFA (3 mL). After stirred overnight, the reaction mixture was concentrated under reduced pressure to give a crude product, which was purified by reverse-phase prep-HPLC to afford compound 4.9 (66 mg) in 77% yield. ¹HNMR (400 MHZ, DMSO-d₆) δ 13.13 (s, 1H), 10.87 (s, 1H), 7.25 (d, J=8.0 Hz, 1H), 7.97 (t, J=8.0 Hz, 1H), 6.79 (d, J=8.0 Hz, 1H), 4.95 (s, 2H), 4.33 (dd, J=5.2, 9.6 Hz, 1H), 4.23 (s, ³H), 2.70-2.64 (m, 1H), 2.63-2.56 (m, 1H), 2.38-2.32 (m, 1H), 2.31-2.15 (m, 1H); MS (ESI) m z: 318.2 [M+1]⁺.

Preparation of N-(3-cyano-4,5-dimethylthiophen-2-yl)-2-((3-(2,6-dioxopiperidin-3-yl)-1-methyl-1H-indazol-7-yl)-oxy)-acetamide B042. Compound B042 was prepared according to the procedure as described in Example 3. MS (ESI) m z: 452.1 [M+1]⁺.

### Example 5

Preparation of 4-amino-5-(4-(2-((3-(2,6-dioxopiperidin-3-yl)-1-methyl-1H-indazol-7-yl)oxy)-acetyl)piperazine-1-carbonyl) isothiazole-3-carboxamide B201

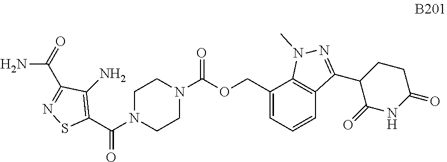 [SMILES: Cn1nc(C2CCC(=O)NC2=O)c2cccc(COC(=O)N3CCN(C(=O)c4snc(C(N)=O)c4N)CC3)c21]

Compound B201 was prepared as shown in Scheme 5.

Preparation of 4-amino-3-carbamoylisothiazole-5-carboxylic acid 5.2. To a solution of ethyl 4-amino-3-carbamoylisothiazole-5-carboxylate 5.1 (43 mg, 0.2 mmol) in THF (1 mL) was added TMSOK (76.8 mg, 0.6 mmol). After stirred at 50° C. for 16 h, the reaction mixture was concentrated under reduced pressure to afford compound 5.2 (43 mg), which was used directly without further purification.

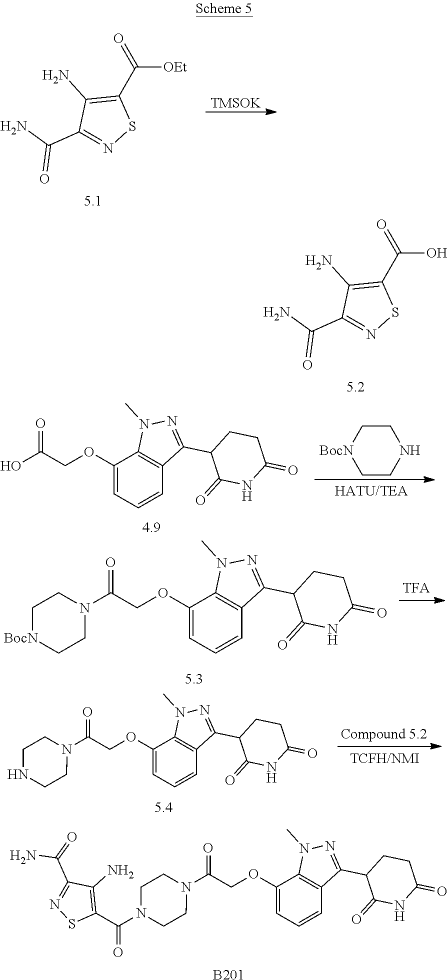

Preparation of tert-butyl 4-(2-((3-(2,6-dioxopiperidin-3-yl)-1-methyl-1H-indazol-7-yl)oxy)acetyl)piperazine-1-carboxylate 5.3. To a solution of 2-((3-(2,6-dioxopiperidin-3-yl)-1-methyl-1H-indazol-7-yl)oxy)acetic acid 4.9 (476 mg, 0.15 mmol) and t-butyl piperazine-1-carboxylate (150 mg, 0.18 mmol) in DMF (10 mL) were added TEA (1.1 mL, 1.5 mmol) and HATU (874 mg, 0.23 mmol). After stirred at 30° C. for 16 h, the reaction mixture was purified by reverse-phase prep-HPLC to afford compound 5.3 (450 mg).

Preparation of 3-(1-methyl-7-(2-oxo-2-(piperazin-1-yl)ethoxy)-1H-indazol-3-yl)-piperidine-2,6-dione 5.4. To a solution of compound 5.3 (450 mg, 0.27 mmol) in DCM (3 mL) was added TFA (3 mL). After stirred overnight, the reaction mixture was concentrated under reduced pressure to yield a crude product, which was purified by reverse-phase prep-HPLC to afford compound 5.4 (350 mg) in 77% yield.

Preparation of 4-amino-5-(4-(2-((3-(2,6-dioxopiperidin-3-yl)-1-methyl-1H-indazol-7-yl)oxy)-acetyl)piperazine-1-carbonyl) isothiazole-3-carboxamide B201. To a solution of compound 5.2 (0.2 mmol) and compound 5.4 (65.5 mg, 0.17 mmol) in ACN (1.5 mL) were added NMI (0.34 mL) and TCFH (70 mg, 0.25 mmol). After stirred at 30° C. for 16 h, the reaction mixture was purified by reverse-phase prep-HPLC to afford compound B201 (20 mg). MS (ESI) m z: 555.2 [M+1]⁺.

### Example 6

### Preparation of 5-(4-(2-((3-(2,6-dioxopiperidin-3-yl)-1-methyl-1H-indazol-7-yl)oxy)acetyl)-piperazine-1-carbonyl)-1H-pyrrole-2-carboxylic acid B202

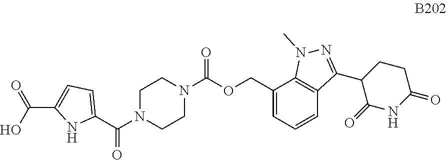 [SMILES: Cn1nc(C2CCC(=O)NC2=O)c2cccc(COC(=O)N3CCN(C(=O)c4ccc(C(=O)O)[nH]4)CC3)c21]

Compound B202 was prepared as shown in Scheme 6.

Preparation of ethyl 5-(4-(2-((3-(2,6-dioxopiperidin-3-yl)-1-methyl-1H-indazol-7-yl)oxy)acetyl)piperazine-1-carbonyl)-1H-pyrrole-2-carboxylate 6.1. To a solution of 3-(1-methyl-7-(2-oxo-2-(piperazin-1-yl)ethoxy)-1H-indazol-3-yl)piperidine-2,6-dione 5.4 (65.5 mg, 0.17 mmol) and 5-(ethoxycarbonyl)-1H-pyrrole-2-carboxylic acid (37.3 mg, 0.204 mmol) in ACN (1.5 mL) were added NMI (0.340 mL) and TCFH (70 mg, 0.25 mmol). After stirred at 30° C. for 16 h, the reaction mixture was purified by reverse-phase prep-HPLC to afford compound 6.1 (45 mg).

Preparation of 5-(4-(2-((3-(2,6-dioxopiperidin-3-yl)-1-methyl-1H-indazol-7-yl)oxy)acetyl)-piperazine-1-carbonyl)-1H-pyrrole-2-carboxylic acid B202. A solution of compound 6.1 (45 mg, 0.08 mmol) in DCM (1 mL) and CF3SO₃H (1 mL) was stirred at 50° C. for 16 h. The reaction mixture was then concentrated under reduced pressure to yield a crude product, which was purified by reverse-phase prep-HPLC to afford compound B202 (17 mg). MS (ESI) m/z: 523.1 [M+1]⁺.

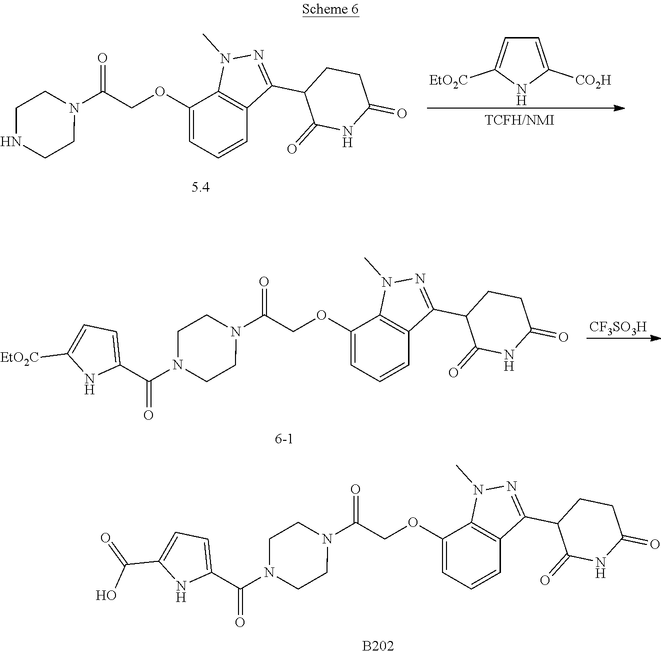

### Example 7

### Preparation of 3-(4-(2-((3-(2,6-dioxopiperidin-3-yl)-1-methyl-1H-indazol-7-yl)oxy)acetyl)-piperazine-1-carbonyl)-1-methyl-1H-pyrazole-5-carboxylic acid B204

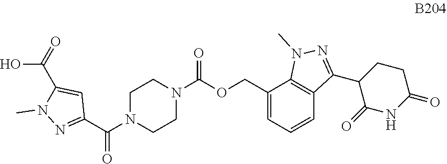 [SMILES: Cn1nc(C(=O)N2CCN(C(=O)OCc3cccc4c(C5CCC(=O)NC5=O)nn(C)c34)CC2)cc1C(=O)O]

Compound B204 was prepared as shown in Scheme 7.

Preparation of methyl 5-(4-(2-((3-(2,6-dioxopiperidin-3-yl)-1-methyl-1H-indazol-7-yl)oxy)acetyl)piperazine-1-carbonyl)-1-methyl-1H-pyrazole-3-carboxylate 7.1. To a solution of 3-(1-methyl-7-(2-oxo-2-(piperazin-1-yl)ethoxy)-1H-indazol-3-yl)piperidine-2,6-dione 5.4 (65.5 mg, 0.17 mmol) and 3-(methoxycarbonyl)-1-methyl-1H-pyrazole-5-carboxylic acid (36.8 mg, 0.2 mmol) in ACN (1.5 mL) were added NMI (0.34 mL) and TCFH (70 mg, 0.25 mmol). After stirred at 30° C. for 16 h, the reaction mixture was purified by reverse-phase prep-HPLC to afford compound 7.1 (24 mg).

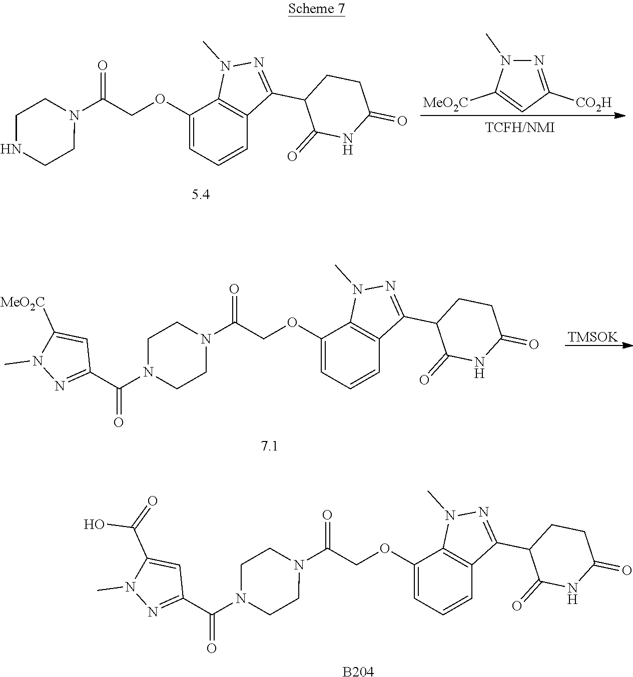 [SMILES: "TMSOK" ; Cn1nc(C2CCC(=O)NC2=O)c2cccc(OCC(=O)N3CCNCC3)c21 ; COC(=O)c1cc(C(=O)O)nn1C ; COC(=O)c1cc(C(=O)N2CCN(C(=O)COc3cccc4c(C5CCC(=O)NC5=O)nn(C)c34)CC2)nn1C ; "B204" ; Cn1nc(C(=O)N2CCN(C(=O)COc3cccc4c(C5CCC(=O)NC5=O)nn(C)c34)CC2)cc1C(=O)O]

Preparation of 3-(4-(2-((3-(2,6-dioxopiperidin-3-yl)-1-methyl-1H-indazol-7-yl)-oxy)acetyl)piperazine-1-carbonyl)-1-methyl-1H-pyrazole-5-carboxylic acid B204. To a solution of compound 7.1 (24 mg, 0.04 mmol) in THF (1 mL) was added TMSOK (15.4 mg, 0.12 mmol). After stirred at 50° C. for 16 h, the reaction mixture was concentrated under reduced pressure to yield a crude product, which was purified by reverse-phase prep HPLC to afford compound B204 (2.4 mg). MS (ESI) m/z: 538.2 [M+1]⁺.

### Example 8

### Preparation of 3-(1-methyl-7-(2-oxo-2-(4-((tetrahydro-2H-pyran-4-yl) sulfonyl)piperazin-1-yl)-ethoxy)-1H-indazol-3-yl)piperidine-2,6-dione B207

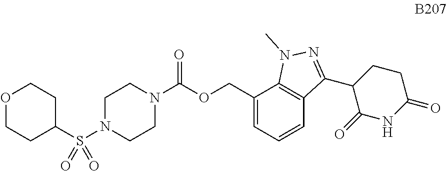 [SMILES: Cn1nc(C2CCC(=O)NC2=O)c2cccc(COC(=O)N3CCN(S(=O)(=O)C4CCOCC4)CC3)c21]

Compound B207 was prepared as shown in Scheme 8.

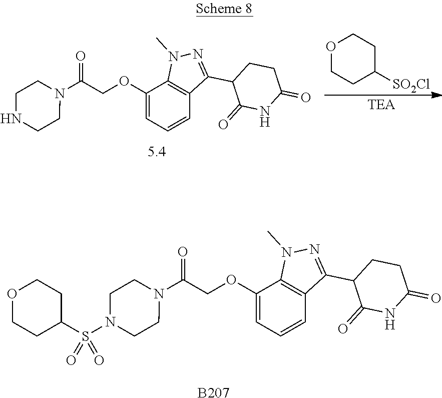

To a solution of 3-(1-methyl-7-(2-oxo-2-(piperazin-1-yl)-ethoxy)-1H-indazol-3-yl)-piperidine-2,6-dione 5.4 (65.5 mg, 0.17 mmol) and tetrahydro-2H-pyran-4-sulfonyl chloride (0.200 mmol) in DCM (2 mL) was added TEA (0.236 mL, 1.7 mmol) under 0° C. After stirred at 30° C. for 16 h, the reaction mixture was concentrated under reduced pressure to yield a crude product, which was purified by reverse-phase prep-HPLC to afford compound B207 (15 mg). MS (ESI) m/z: 534.2 [M+1]⁺.

### Example 9

### Preparation of 3-(7-(2-(4-((3,5-dimethylisoxazol-4-yl) sulfonyl)piperazin-1-yl)-2-oxoethoxy)-1-methyl-1H-indazol-3-yl)piperidine-2,6-dione B215

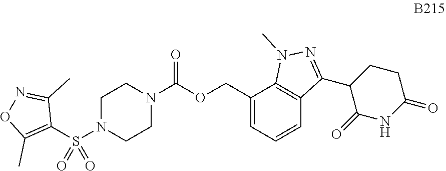 [SMILES: Cc1noc(C)c1S(=O)(=O)N1CCN(C(=O)OCc2cccc3c(C4CCC(=O)NC4=O)nn(C)c23)CC1]

Compound B215 was prepared as shown in Scheme 9.

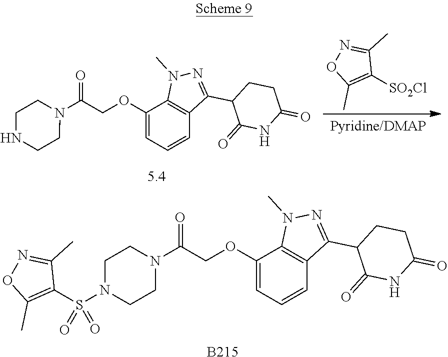

To a solution of 3-(1-methyl-7-(2-oxo-2-(piperazin-1-yl)ethoxy)-1H-indazol-3-yl)piperidine-2,6-dione 5.4 (65.5 mg, 0.17 mmol.) and 3,5-dimethylisoxazole-4-sulfonyl chloride (0.25 mmol) in DCM (1.5 mL) were added pyridine (0.236 mL, 1.7 mmol) and DMAP (20.8 mg, 0.17 mmol) under 0° C. After stirred at 50° C. for 16 h, the reaction mixture was concentrated under reduced pressure to yield a crude product, which was purified by reverse-phase prep-HPLC to afford compound B215 (25 mg). MS (ESI) m/z: 545.2 [M+1]⁺.

### Example 10

### Preparation of 3-(7-(2-(4-(1,2,4-oxadiazole-3-carbonyl)piperazin-1-yl)-2-oxoethoxy)-1-methyl-1H-indazol-3-yl)piperidine-2,6-dione B219

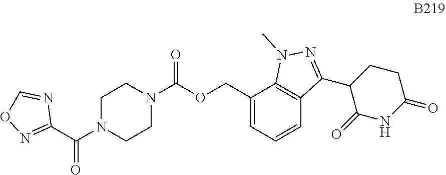 [SMILES: Cn1nc(C2CCC(=O)NC2=O)c2cccc(COC(=O)N3CCN(C(=O)c4ncon4)CC3)c21]

Compound B219 was prepared as shown in Scheme 10.

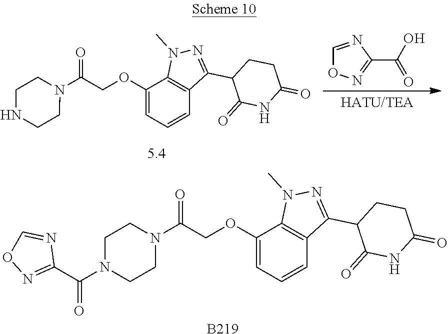

To a solution of 3-(1-methyl-7-(2-oxo-2-(piperazin-1-yl)ethoxy)-1H-indazol-3-yl)piperidine-2,6-dione 5.4 (65.5 mg, 0.17 mmol) and 1,2,4-oxadiazole-3-carboxylic acid (23.3 mg, 0.204 mmol) in DMF (1.5 mL) were added TEA (0.236 mL, 1.7 mmol) and HATU (98.8 mg, 0.26 mmol). After stirred at 30° C. for 16 h, the reaction mixture was purified by reverse-phase prep-HPLC to afford compound B219 (30 mg). MS (ESI) m/z: 482.2 [M+1]⁺.

### Example 11

### Preparation of 3-(7-(2-(4-(4-amino-1,2,5-oxadiazole-3-carbonyl)piperazin-1-yl)-2-oxoethoxy)-1-methyl-1H-indazol-3-yl)piperidine-2,6-dione B233

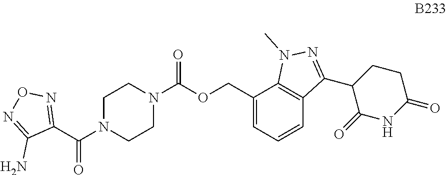 [SMILES: Cn1nc(C2CCC(=O)NC2=O)c2cccc(COC(=O)N3CCN(C(=O)c4nonc4N)CC3)c21]

Compound B233 was prepared as shown in Scheme 11.

To a solution of 3-(1-methyl-7-(2-oxo-2-(piperazin-1-yl)ethoxy)-1H-indazol-3-yl)piperidine-2,6-dione 5.4 (65.5 mg, 0.170 mmol) and 4-amino-1,2,5-oxadiazole-3-carboxylic acid (26.3 mg, 0.204 mmol) in ACN (1.5 mL) were added NMI (0.340 mL) and TCFH (70 mg, 0.25 mmol). After stirred at 30° C. for 16 h, the reaction mixture was purified by reverse-phase prep-HPLC to afford compound B233 (38.6 mg). MS (ESI) m/z: 497.2 [M+1]⁺.

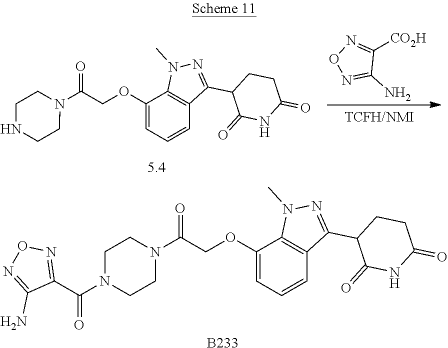 [SMILES: "B233" ; Cn1nc(C2CCC(=O)NC2=O)c2cccc(OCC(=O)N3CCN(C(=O)c4nonc4N)CC3)c21 ; Cn1nc(C2CCC(=O)NC2=O)c2cccc(OCC(=O)N3CCNCC3)c21 ; Nc1nonc1C(=O)O]

### Example 12

### Preparation of 3-(7-(2-(4-(4-amino-1-methyl-1H-pyrazole-5-carbonyl)piperazin-1-yl)-2-oxo-ethoxy)-1-methyl-1H-indazol-3-yl)piperidine-2,6-dione B242

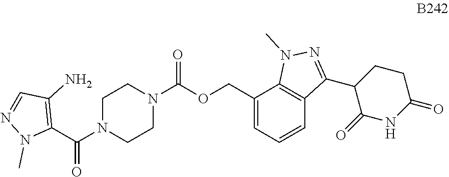 [SMILES: Cn1ncc(N)c1C(=O)N1CCN(C(=O)OCc2cccc3c(C4CCC(=O)NC4=O)nn(C)c23)CC1]

Compound B242 was prepared as shown in Scheme 12.

To a solution of 3-(1-methyl-7-(2-oxo-2-(piperazin-1-yl)ethoxy)-1H-indazol-3-yl)-piperidine-2,6-dione 5.4 (65.5 mg, 0.17 mmol) and 4-amino-1-methyl-1H-pyrazole-5-carboxylic acid (28.8 mg, 0.204 mmol) in DCM (1.5 mL) were added DIPEA (0.236 mL, 1.7 mmol), DMAP (20.7 mg, 0.17 mmol), and 50% T₄P in EtOAc (180 μL). After stirred at 50° C. for 16 h, the reaction mixture was concentrated under reduced pressure to yield a crude product, which was purified by reverse-phase prep-HPLC to afford compound B242 (5.1 mg). MS (ESI) m/z: 509.2 [M+1]⁺.

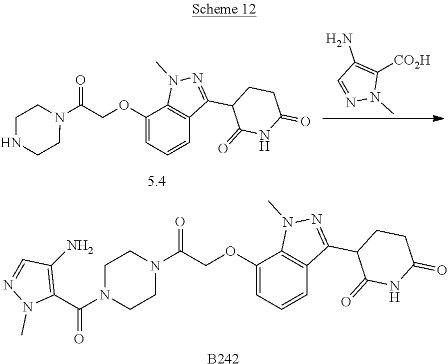

### Example 13

### Preparation of 3-(1-methyl-7-((1-(oxetane-3-carbonyl)piperidin-4-yl)oxy)-1H-indazol-3-yl)-piperidine-2,6-dione C001

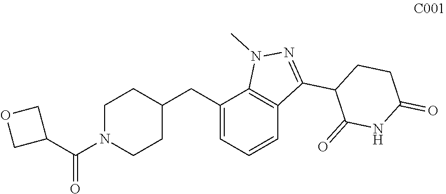 [SMILES: Cn1nc(C2CCC(=O)NC2=O)c2cccc(CC3CCN(C(=O)C4COC4)CC3)c21]

Compound C001 was prepared as shown in Scheme 13.

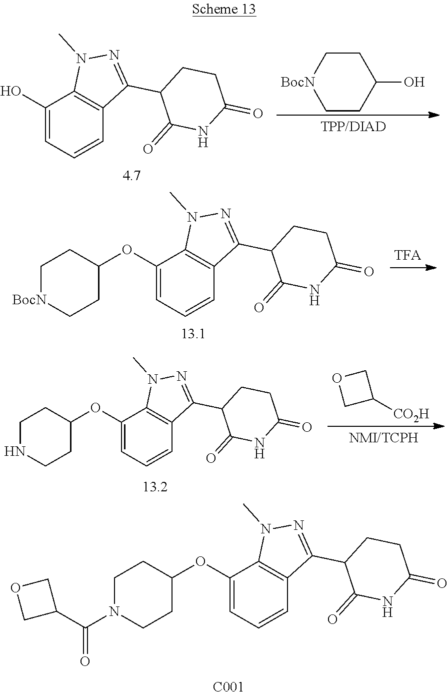

Preparation of tert-butyl 4-((3-(2,6-dioxopiperidin-3-yl)-1-methyl-1H-indazol-7-yl)oxy) piperidine-1-carboxylate 13.1. To a solution of 3-(7-hydroxy-1-methyl-1H-indazol-3-yl)-piperidine-2,6-dione 4.7 (5 g, 19.7 mmol) in ACN (30 mL) were added DIAD (3.5 mL, 29.6 mmol) and TPP (3.7 g, 25.6 mmol). After stirred overnight, the reaction mixture was concentrated under reduced pressure to yield a crude product, which was purified by silica-gel column chromatography eluting with EtOAc/PE to afford 13.1 (1.8 g) in 21% yield. MS (ESI) m z: 443.2 [M+1]⁺.

Preparation of 3-(1-methyl-7-(piperidin-4-yloxy)-1H-indazol-3-yl)piperidine-2,6-dione 13.2. To a solution of compound 13.1 (8.5 g, 18.7 mmol) in DCM (150 mL) was added TFA (75 mL). After stirred at 30° C. for 2 h, the reaction mixture was concentrated under reduced pressure to afford compound 13.2 as a TFA salt (8.35 g), which was used directly in the next step without further purification.

Preparation of 3-(1-methyl-7-((1-(oxetane-3-carbonyl)piperidin-4-yl)oxy)-1H-indazol-3-yl)-piperidine-2,6-dione C001. To a solution of compound 13.2 (45.5 mg, 0.1 mmol) and 4-amino-1,2,5-oxadiazole-3-carboxylic acid (26.3 mg, 0.15 mmol) in ACN (1.5 mL) were added NMI (0.1 mL) and TCFH (42.1 mg, 0.15 mmol). After stirred at 30° C. for 16 h, the reaction mixture was purified by reverse-phase prep-HPLC to afford compound C001 (38.6 mg). MS (ESI) m z: 427.2 [M+1]⁺.

### Example 14

### Preparation of N-(3-cyclopropyl-1H-pyrazol-5-yl)-2-(3-(2,6-dioxopiperidin-3-yl)-1H-indazol-1-yl)acetamide D001

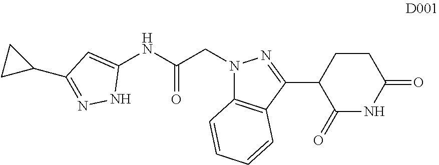 [SMILES: O=C1CCC(c2nn(CC(=O)Nc3cc(C4CC4)n[nH]3)c3ccccc23)C(=O)N1]

Compound D001 was prepared as shown in Scheme 14.

Preparation of tert-butyl 2-(3-bromo-1H-indazol-1-yl)-acetate 14.2. To a solution of 3-bromo-1H-indazole 14.1 (500 mg, 2.55 mmol) and tert-butyl 2-bromoacetate (594 mg, 3.06 mmol) in DMF (40 mL) was added K₂CO₃ (880 mg, 6.38 mmol). After stirred overnight, the reaction mixture was diluted with water (10 mL) and extracted with EtOAc (15 mL×3). The combined organic layers were dried over anhydrous Na₂SO₄, filtered, and concentrated under reduced pressure to afford 14.2 (769 mg), which was used directly in the next step without further purification. MS (ESI) m/z: 333.1 [M+Na]+.

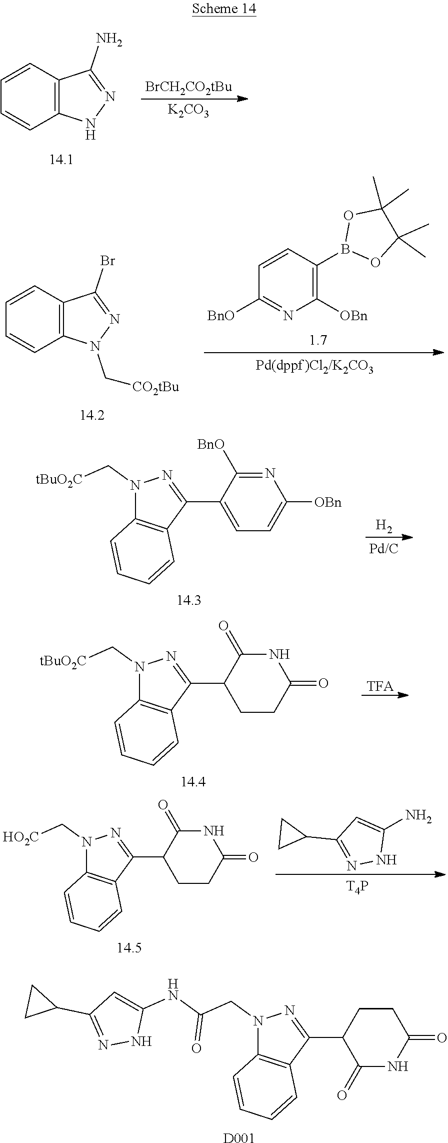

Preparation of tert-butyl-2-(3-(2,6-bis-(benzyloxy)-pyridin-3-yl)-1H-indazol-1-yl)-acetate 14.3. To a solution of 2,6-bis(benzyloxy)-3-(4,4,5,5-tetramethyl-1,3,2-dioxaborolan-2-yl)pyridine 1.7 (1.1 mg, 2.64 mmol) and compound 14.2 (630 mg, 2.03 mmol) in 1,4-dioxane/water (6:1, 30 mL) were added K₂CO₃ (560 mg, 4.06 mmol) and Pd (dppf) Cl₂ (146 mg, 0.2 mmol) under N₂. After heated at 90° C. under N₂ for 3.5 h, the reaction mixture was diluted with water (100 mL) and extracted with EtOAc (100 mL×3). The combined organic layers were dried over anhydrous Na₂SO₄, filtered, and concentrated under reduced pressure to yield a crude product, which was purified by silica-gel column chromatography eluting with EtOAc/PE to give afford compound 14.3 (998 mg) in 94% yield. MS (ESI) m/z: 522.1 [M+1]⁺.

Preparation of tert-butyl 2-(3-(2,6-dioxopiperidin-3-yl)-1H-indazol-1-yl)acetate 14.4. To a solution of compound 14.3 (998 mg, 1.92 mmol) in MeOH (25 mL) and THF (25 mL) was added 10% Pd/C (200 mg) under N₂. After stirred under H₂ overnight, the reaction mixture was filtered and concentrated to afford compound 14.4 (617 mg) in 94% yield. MS (ESI) m z: 344.2 [M+1]⁺.

Preparation of 2-(3-(2,6-dioxopiperidin-3-yl)-1H-indazol-1-yl)acetic acid 14.5. To a solution of compound 14.4 (150 mg, 0.44 mmol) in DCM (3 mL) was added TFA (1 mL). After stirred overnight, the reaction mixture was concentrated under reduced pressure, and then redissolved with DCM and MeOH, adjusted pH 7 with NaHCO₃, filtered, and concentrated under reduced pressure to yield a crude product, which was purified by reverse-phase prep-HPLC to afford compound 14.5 (41 mg) in 33% yield. ¹H NMR (400 MHZ, DMSO-d₆) δ 13.04 (s, 1H), 10.91 (s, 1H), 7.73 (d, J=8.0 Hz, 1H), 7.60 (d, J=8.0 Hz, 1H), 7.39 (td, J=0.8, 7.2 Hz, 1H), 7.14 (t, J=7.2 Hz, 1H), 5.21 (d, J=2.0 Hz, 2H), 4.40 (dd, J=5.2, 9.6 Hz, 1H), 2.68-2.52 (m, 2H), 2.38-2.33 (m, 1H), 2.21-2.16 (m, 1H); MS (ESI) m z: 288.1 [M+1]⁺.

Preparation of N-(3-cyclopropyl-1H-pyrazol-5-yl)-2-(3-(2,6-dioxopiperidin-3-yl)-1H-indazol-1-yl)acetamide D001. To a solution of compound 14.5 (57.4 mg, 0.2 mmol) and 3-cyclopropyl-1H-pyrazol-5-amine (29.52 mg, 0.24 mmol) in THF (0.6 mL) and pyridine (0.6 mL) was added 50% T₄P in EtOAc (0.4 mL). After stirred at 80° C. for 16 h, the reaction mixture was concentrated under reduced pressure to yield a crude product, which was purified by reverse-phase prep-HPLC to afford compound D001 (6 mg). MS (ESI) m z: 393.1 [M+1]⁺.

### Example 15

### Preparation of N-(4-(1H-imidazol-5-yl)phenyl)-2-(3-(2,6-dioxopiperidin-3-yl)-1H-indazol-1-yl)-acetamide D003

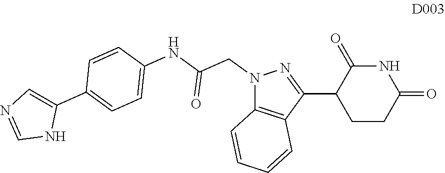 [SMILES: O=C1CCC(c2nn(CC(=O)Nc3ccc(-c4cnc[nH]4)cc3)c3ccccc23)C(=O)N1]

Compound D003 was prepared as shown in Scheme 15.

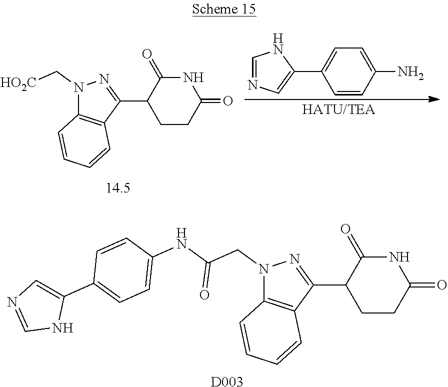

To a solution of 2-(3-(2,6-dioxopiperidin-3-yl)-1H-indazol-1-yl)acetic acid 14.5 (57.4 mg, 0.2 mmol) and 4-(1H-imidazol-5-yl) aniline (38.2 mg, 0.24 mmol) in DMF (1 mL) were added TEA (0.138 mL, 1 mmol) and HATU (45.6 mg, 0.24 mmol) in DMF (0.5 mL). After stirred at 50° C. for 16 h, the reaction mixture was purified by reverse-phase prep-HPLC to afford compound D003 (4.5 mg). MS (ESI) m/z: 429.2 [M+1]⁺.

### Example 16

### Preparation of N-cyclopropyl-2-(3-(2,6-dioxopiperidin-3-yl)-1H-indazol-1-yl)acetamide D049

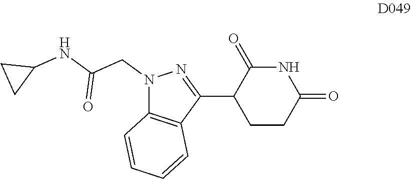 [SMILES: O=C1CCC(c2nn(CC(=O)NC3CC3)c3ccccc23)C(=O)N1]

Compound D049 was prepared as shown in Scheme 16.

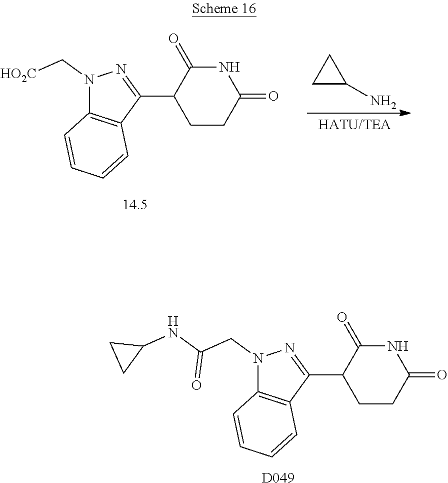

To a solution of 2-(3-(2,6-dioxopiperidin-3-yl)-1H-indazol-1-yl)acetic acid 14.5 (57.4 mg, 0.2 mmol) and cyclopropanamine (13.7 mg, 0.24 mmol) in ACN (2 mL) were added NMI (0.5 mL) and TCFH (67.2 mg, 0.24 mmol). After stirred at 30° C. for 16 h, the reaction mixture was concentrated under reduced pressure to yield a crude product, which was purified by reverse-phase prep-HPLC to afford compound D049 (5.5 mg). MS (ESI) m/z: 327.2 [M+1]⁺.

The following compounds were prepared similarly according to the synthetic procedures or methodologies exemplified herein.

2-((3-(2,6-Dioxopiperidin-3-yl)-1-methyl-1H-indazol-6-yl)oxy)-N-(oxazol-2-yl)-acetamide A001. MS (ESI) m/z: 384.1 [M+1]⁺.

2-((3-(2,6-Dioxopiperidin-3-yl)-1-methyl-1H-indazol-6-yl)oxy)-N-((1s,4s)-4-hydroxycyclohexyl)acetamide A002. MS (ESI) m/z: 415.1 [M+1]⁺.

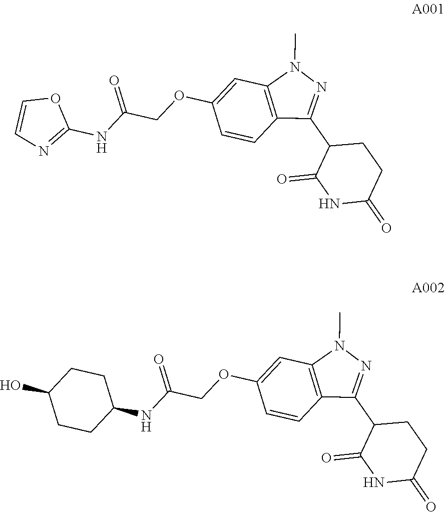 [SMILES: Cn1nc(C2CCC(=O)NC2=O)c2ccc(OCC(=O)Nc3ncco3)cc21 ; Cn1nc(C2CCC(=O)NC2=O)c2ccc(OCC(=O)N[C@H]3CC[C@@H](O)CC3)cc21]

2-((3-(2,6-Dioxopiperidin-3-yl)-1-methyl-1H-indazol-6-yl)oxy)-N-(3-fluoro-5-morpholinophenyl)acetamide A003. MS (ESI) m/z: 496.3 [M+1]⁺.

N-(4-Cyano-3-methylisoxazol-5-yl)-2-((3-(2,6-dioxopiperidin-3-yl)-1-methyl-1H-indazol-6-yl)oxy)acetamide A004. MS (ESI) m/z: 423.1 [M+1]⁺.

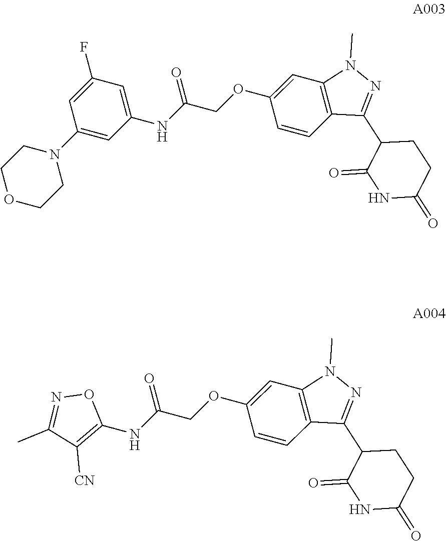 [SMILES: Cn1nc(C2CCC(=O)NC2=O)c2ccc(OCC(=O)Nc3cc(F)cc(N4CCOCC4)c3)cc21 ; Cc1noc(NC(=O)COc2ccc3c(C4CCC(=O)NC4=O)nn(C)c3c2)c1C#N]

2-((3-(2,6-Dioxopiperidin-3-yl)-1-methyl-1H-indazol-6-yl)oxy)-N-((1-methyl-1H-imidazol-5-yl)methyl)acetamide A005. MS (ESI) m/z: 411.2 [M+1]⁺.

N-(1-Benzylpyrrolidin-3-yl)-2-((3-(2,6-dioxopiperidin-3-yl)-1-methyl-1H-indazol-6-yl)oxy)acetamide A006. MS (ESI) m/z: 476.2 [M+1]⁺.

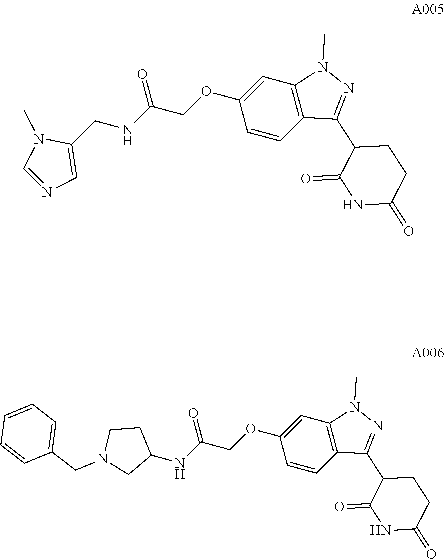 [SMILES: Cn1cncc1CNC(=O)COc1ccc2c(C3CCC(=O)NC3=O)nn(C)c2c1 ; Cn1nc(C2CCC(=O)NC2=O)c2ccc(OCC(=O)NC3CCN(Cc4ccccc4)C3)cc21]

N-(3-Cyclopropyl-1-methyl-1H-pyrazol-5-yl)-2-((3-(2,6-dioxopiperidin-3-yl)-1-methyl-1H-indazol-6-yl)oxy)acetamide A007. MS (ESI) m/z: 437.2 [M+1]⁺.

2-((3-(2,6-Dioxopiperidin-3-yl)-1-methyl-1H-indazol-6-yl)oxy)-N-(2-sulfamoylethyl)acetamide A008. MS (ESI) m/z: 424.1 [M+1]⁺.

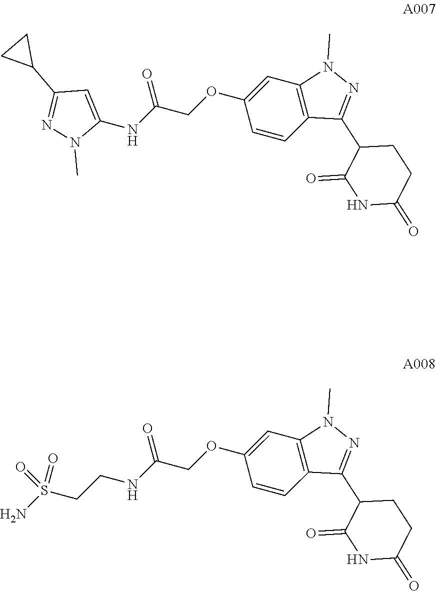 [SMILES: Cn1nc(C2CC2)cc1NC(=O)COc1ccc2c(C3CCC(=O)NC3=O)nn(C)c2c1 ; Cn1nc(C2CCC(=O)NC2=O)c2ccc(OCC(=O)NCCS(N)(=O)=O)cc21]

2-((3-(2,6-Dioxopiperidin-3-yl)-1-methyl-1H-indazol-6-yl)oxy)-N-((1-hydroxy-cyclopropyl)methyl)acetamide A009. MS (ESI) m/z: 387.2 [M+1]⁺.

2-((3-(2,6-Dioxopiperidin-3-yl)-1-methyl-1H-indazol-6-yl)oxy)-N-(1-methyl-1H-pyrazol-4-yl)acetamide A010. MS (ESI) m/z: 397.2 [M+1]⁺.

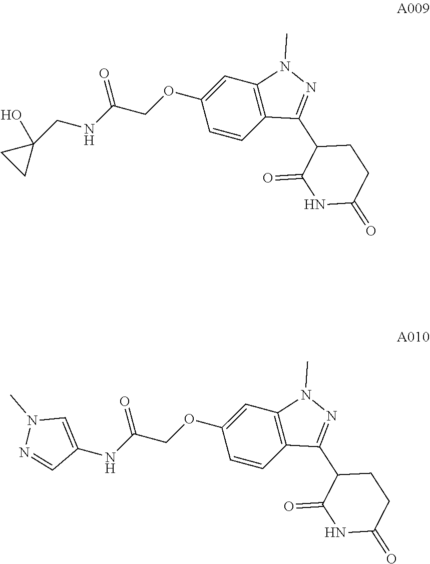 [SMILES: Cn1nc(C2CCC(=O)NC2=O)c2ccc(OCC(=O)NCC3(O)CC3)cc21 ; Cn1cc(NC(=O)COc2ccc3c(C4CCC(=O)NC4=O)nn(C)c3c2)cn1]

2-((3-(2,6-Dioxopiperidin-3-yl)-1-methyl-1H-indazol-6-yl)oxy)-N-((1-methyl-1H-pyrazol-3-yl)methyl)acetamide A011. MS (ESI) m/z: 411.2 [M+1]⁺.

2-((3-(2,6-Dioxopiperidin-3-yl)-1-methyl-1H-indazol-6-yl)oxy)-N-(2-(p-tolyl-oxy)ethyl)acetamide A013. MS (ESI) m/z: 451.2 [M+1]⁺.

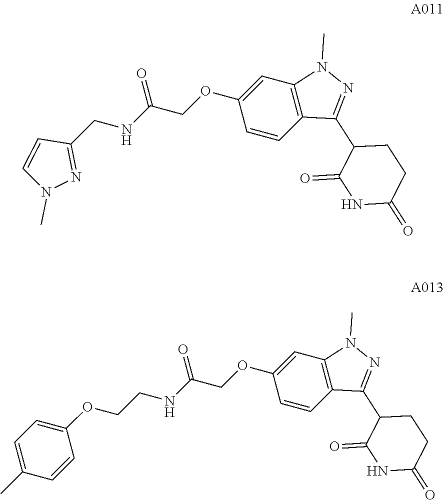 [SMILES: Cn1ccc(CNC(=O)COc2ccc3c(C4CCC(=O)NC4=O)nn(C)c3c2)n1 ; Cc1ccc(OCCNC(=O)COc2ccc3c(C4CCC(=O)NC4=O)nn(C)c3c2)cc1]

N-((4-Benzylmorpholin-2-yl)methyl)-2-((3-(2,6-dioxopiperidin-3-yl)-1-methyl-1H-indazol-6-yl)oxy)acetamide A014. MS (ESI) m/z: 506.2 [M+1]⁺.

2-((3-(2,6-Dioxopiperidin-3-yl)-1-methyl-1H-indazol-6-yl)oxy)-N-(1-phenyl-1H-pyrazol-4-yl)acetamide A015. MS (ESI) m/z: 459.2 [M+1]⁺.

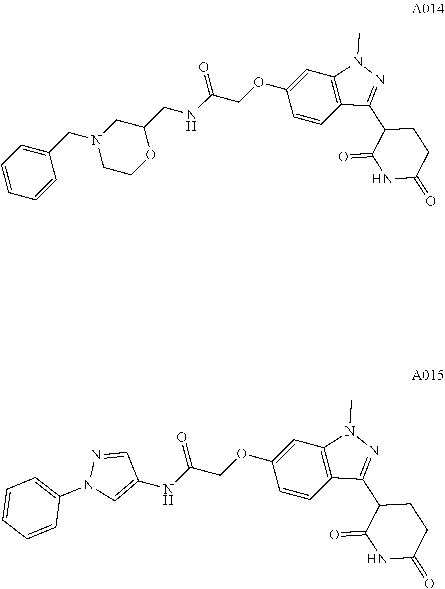 [SMILES: Cn1nc(C2CCC(=O)NC2=O)c2ccc(OCC(=O)NCC3CN(Cc4ccccc4)CCO3)cc21 ; Cn1nc(C2CCC(=O)NC2=O)c2ccc(OCC(=O)Nc3cnn(-c4ccccc4)c3)cc21]

2-((3-(2,6-Dioxopiperidin-3-yl)-1-methyl-1H-indazol-6-yl)oxy)-N-(thiazol-2-yl-methyl)acetamide A016. MS (ESI) m/z: 414.1 [M+1]⁺.

2-((3-(2,6-Dioxopiperidin-3-yl)-1-methyl-1H-indazol-6-yl)oxy)-N-(4-((pyrrolidin-1-ylsulfonyl)methyl)phenyl)acetamide A017. MS (ESI) m/z: 540.2 [M+1]⁺.

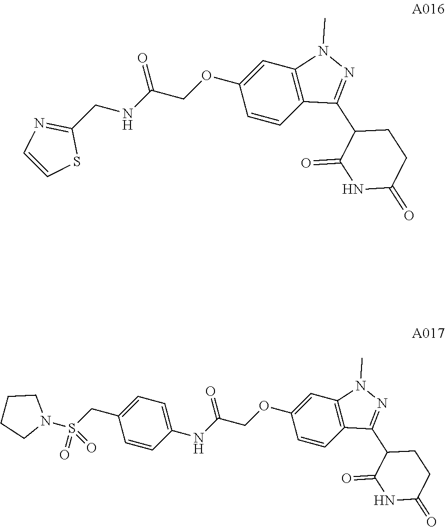 [SMILES: Cn1nc(C2CCC(=O)NC2=O)c2ccc(OCC(=O)NCc3nccs3)cc21 ; Cn1nc(C2CCC(=O)NC2=O)c2ccc(OCC(=O)Nc3ccc(CS(=O)(=O)N4CCCC4)cc3)cc21]

N-(3,5-Dimethylisoxazol-4-yl)-2-((3-(2,6-dioxopiperidin-3-yl)-1-methyl-1H-indazol-6-yl)oxy)acetamide A018. MS (ESI) m/z: 412.1 [M+1]⁺.

2-((3-(2,6-Dioxopiperidin-3-yl)-1-methyl-1H-indazol-6-yl)oxy)-N-(2-(methyl-amino)-2-oxoethyl)acetamide A019. MS (ESI) m/z: 388.2 [M+1]⁺.

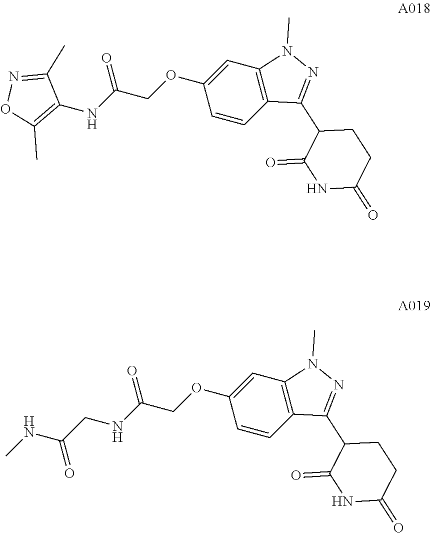 [SMILES: Cc1noc(C)c1NC(=O)COc1ccc2c(C3CCC(=O)NC3=O)nn(C)c2c1 ; CNC(=O)CNC(=O)COc1ccc2c(C3CCC(=O)NC3=O)nn(C)c2c1]

2-((3-(2,6-Dioxopiperidin-3-yl)-1-methyl-1H-indazol-6-yl)oxy)-N-(4-(N-methylsulfamoyl)phenyl)acetamide A020. MS (ESI) m/z: 486.1 [M+1]⁺.

2-((3-(2,6-Dioxopiperidin-3-yl)-1-methyl-1H-indazol-6-yl)oxy)-N-(1-methyl-1H-1,2,4-triazol-5-yl)acetamide A021. MS (ESI) m/z: 398.1 [M+1]⁺.

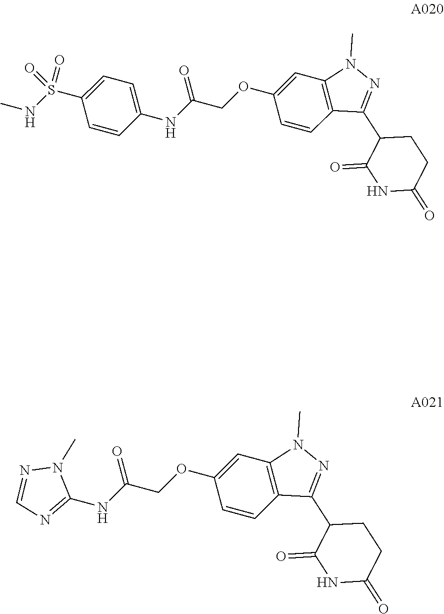 [SMILES: CNS(=O)(=O)c1ccc(NC(=O)COc2ccc3c(C4CCC(=O)NC4=O)nn(C)c3c2)cc1 ; Cn1ncnc1NC(=O)COc1ccc2c(C3CCC(=O)NC3=O)nn(C)c2c1]

2-((3-(2,6-Dioxopiperidin-3-yl)-1-methyl-1H-indazol-6-yl)oxy)-N-(4-(methyl-sulfonyl)phenyl)acetamide A022. MS (ESI) m/z: 471.1 [M+1]⁺.

2-((3-(2,6-Dioxopiperidin-3-yl)-1-methyl-1H-indazol-6-yl)oxy)-N-(4-sulfamoyl-phenethyl)acetamide A023. MS (ESI) m/z: 500.2 [M+1]⁺.

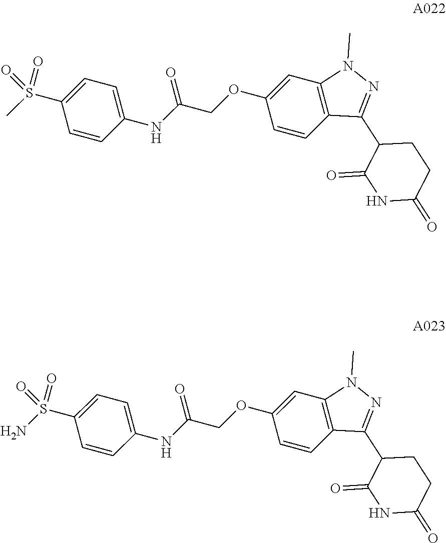 [SMILES: Cn1nc(C2CCC(=O)NC2=O)c2ccc(OCC(=O)Nc3ccc(S(C)(=O)=O)cc3)cc21 ; Cn1nc(C2CCC(=O)NC2=O)c2ccc(OCC(=O)Nc3ccc(S(N)(=O)=O)cc3)cc21]

2-((3-(2,6-Dioxopiperidin-3-yl)-1-methyl-1H-indazol-6-yl)oxy)-N-(4-(1-methyl-piperidin-4-yl)phenyl)acetamide A024. MS (ESI) m/z: 490.2 [M+1]⁺.

2-((3-(2,6-Dioxopiperidin-3-yl)-1-methyl-1H-indazol-6-yl)oxy)-N-((6-ethoxy-pyridin-3-yl)methyl)acetamide A025. MS (ESI) m/z: 452.2 [M+1]⁺.

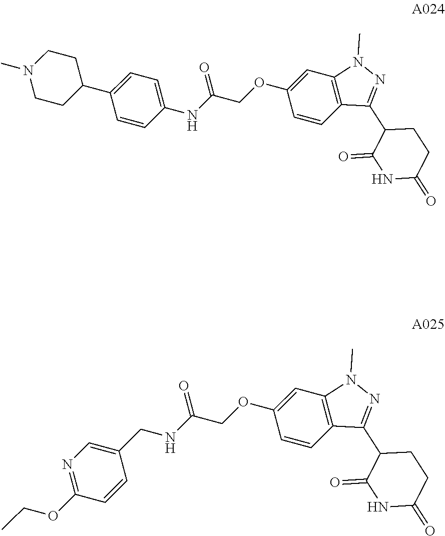 [SMILES: CN1CCC(c2ccc(NC(=O)COc3ccc4c(C5CCC(=O)NC5=O)nn(C)c4c3)cc2)CC1 ; CCOc1ccc(CNC(=O)COc2ccc3c(C4CCC(=O)NC4=O)nn(C)c3c2)cn1]

2-((3-(2,6-Dioxopiperidin-3-yl)-1-methyl-1H-indazol-6-yl)oxy)-N-(pyridazin-3-yl)acetamide A026. MS (ESI) m/z: 395.1 [M+1]⁺.

2-((3-(2,6-Dioxopiperidin-3-yl)-1-methyl-1H-indazol-6-yl)oxy)-N-(pyrimidin-5-yl)acetamide A028. MS (ESI) m/z: 395.1 [M+1]⁺.

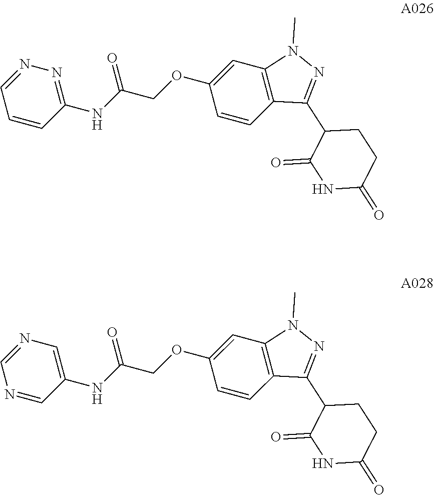 [SMILES: Cn1nc(C2CCC(=O)NC2=O)c2ccc(OCC(=O)Nc3cccnn3)cc21 ; Cn1nc(C2CCC(=O)NC2=O)c2ccc(OCC(=O)Nc3cncnc3)cc21]

2-((3-(2,6-Dioxopiperidin-3-yl)-1-methyl-1H-indazol-6-yl)oxy)-N-(1-methyl-azetidin-3-yl)acetamide A029. MS (ESI) m/z: 386.2 [M+1]⁺.

2-((3-(2,6-Dioxopiperidin-3-yl)-1-methyl-1H-indazol-6-yl)oxy)-N-(3-methyl-isoxazol-5-yl)acetamide A030. MS (ESI) m/z: 398.2 [M+1]⁺.

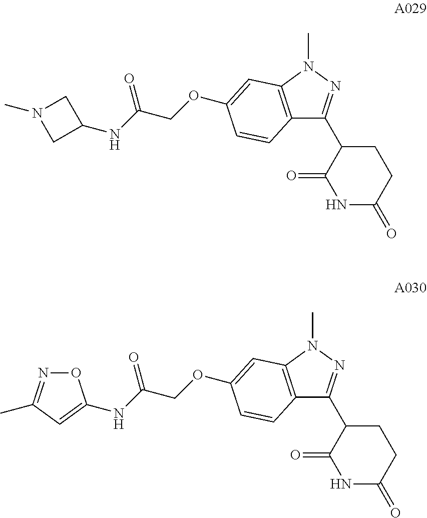 [SMILES: CN1CC(NC(=O)COc2ccc3c(C4CCC(=O)NC4=O)nn(C)c3c2)C1 ; Cc1cc(NC(=O)COc2ccc3c(C4CCC(=O)NC4=O)nn(C)c3c2)on1]

N-(4-(Tert-butyl) thiazol-2-yl)-2-((3-(2,6-dioxopiperidin-3-yl)-1-methyl-1H-indazol-6-yl)oxy)acetamide A031. MS (ESI) m/z: 456.2 [M+1]⁺.

2-((3-(2,6-Dioxopiperidin-3-yl)-1-methyl-1H-indazol-6-yl)oxy)-N-(2-methyl-thiazol-5-yl)acetamide A032. MS (ESI) m/z: 414.1 [M+1]⁺.

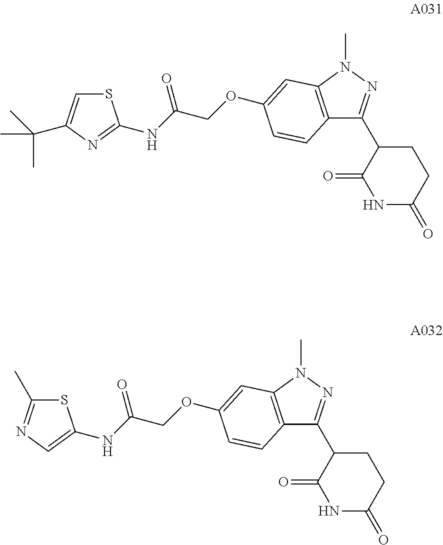 [SMILES: Cn1nc(C2CCC(=O)NC2=O)c2ccc(OCC(=O)Nc3nc(C(C)(C)C)cs3)cc21 ; Cc1ncc(NC(=O)COc2ccc3c(C4CCC(=O)NC4=O)nn(C)c3c2)s1]

2-((3-(2,6-Dioxopiperidin-3-yl)-1-methyl-1H-indazol-6-yl)oxy)-N-(3-ethyl-isoxazol-5-yl)acetamide A0033. MS (ESI) m/z: 412.1 [M+1]⁺.

2-((3-(2,6-Dioxopiperidin-3-yl)-1-methyl-1H-indazol-6-yl)oxy)-N-(3-methyl-isothiazol-5-yl)acetamide A0034. MS (ESI) m/z: 414.1 [M+1]⁺.

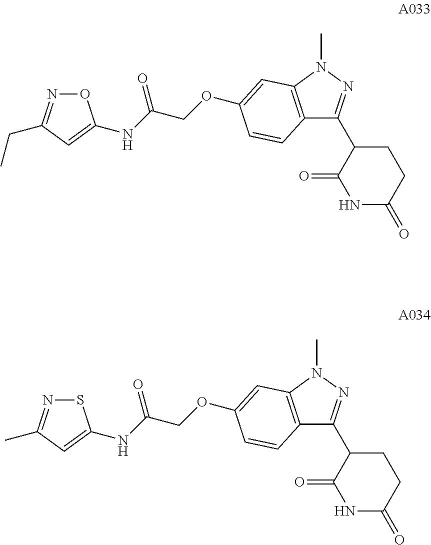 [SMILES: CCc1cc(NC(=O)COc2ccc3c(C4CCC(=O)NC4=O)nn(C)c3c2)on1 ; Cc1cc(NC(=O)COc2ccc3c(C4CCC(=O)NC4=O)nn(C)c3c2)sn1]

2-((3-(2,6-Dioxopiperidin-3-yl)-1-methyl-1H-indazol-6-yl)oxy)-N-(thiazol-4-yl)-acetamide A035. MS (ESI) m/z: 400.2 [M+1]⁺.

2-((3-(2,6-Dioxopiperidin-3-yl)-1-methyl-1H-indazol-6-yl)oxy)-N-(3-methyl-isothiazol-4-yl)acetamide A036. MS (ESI) m/z: 414.1 [M+1]⁺.

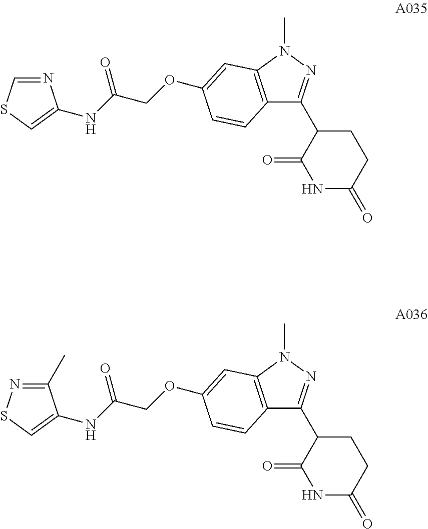 [SMILES: Cn1nc(C2CCC(=O)NC2=O)c2ccc(OCC(=O)Nc3cscn3)cc21 ; Cc1nscc1NC(=O)COc1ccc2c(C3CCC(=O)NC3=O)nn(C)c2c1]

N-((1,5-Dimethyl-1H-pyrazol-3-yl)methyl)-2-((3-(2,6-dioxopiperidin-3-yl)-1-methyl-1H-indazol-6-yl)oxy)acetamide A037. MS (ESI) m/z: 425.2 [M+1]⁺.

N-(3-(1,1-Dioxidothiomorpholino) propyl)-2-((3-(2,6-dioxopiperidin-3-yl)-1-methyl-1H-indazol-6-yl)oxy)acetamide A038. MS (ESI) m/z: 492.2 [M+1]⁺.

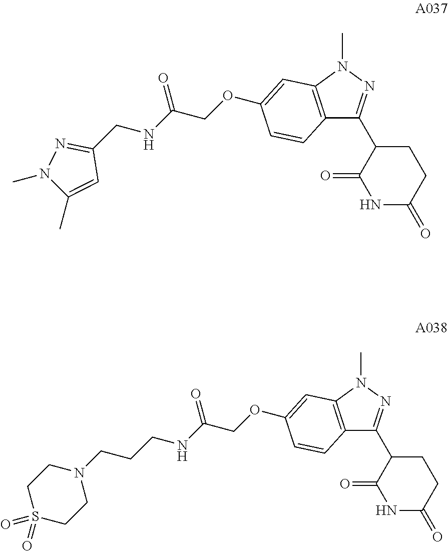 [SMILES: Cc1cc(CNC(=O)COc2ccc3c(C4CCC(=O)NC4=O)nn(C)c3c2)nn1C ; Cn1nc(C2CCC(=O)NC2=O)c2ccc(OCC(=O)NCCCN3CCS(=O)(=O)CC3)cc21]

2-((3-(2,6-Dioxopiperidin-3-yl)-1-methyl-1H-indazol-6-yl)oxy)-N-(tetrahydro-furan-3-yl)acetamide A039. MS (ESI) m/z: 387.2 [M+1]⁺.

2-((3-(2,6-Dioxopiperidin-3-yl)-1-methyl-1H-indazol-6-yl)oxy)-N—((R)-1-(pyridin-2-yl)ethyl)acetamide A040. MS (ESI) m/z: 422.2 [M+1]⁺.

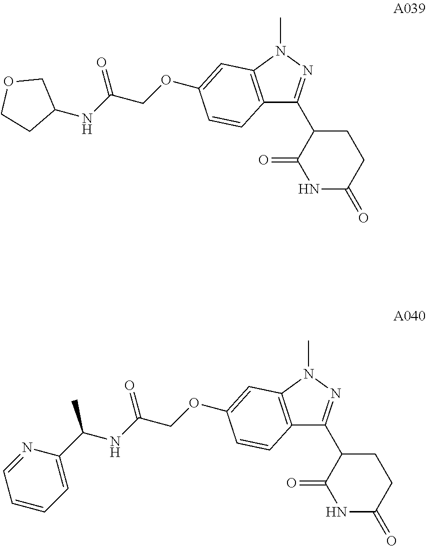 [SMILES: Cn1nc(C2CCC(=O)NC2=O)c2ccc(OCC(=O)NC3CCOC3)cc21 ; C[C@@H](NC(=O)COc1ccc2c(C3CCC(=O)NC3=O)nn(C)c2c1)c1ccccn1]

2-((3-(2,6-Dioxopiperidin-3-yl)-1-methyl-1H-indazol-6-yl)oxy)-N-(oxetan-3-yl)-acetamide A041. MS (ESI) m/z: 373.2 [M+1]⁺.

2-((3-(2,6-Dioxopiperidin-3-yl)-1-methyl-1H-indazol-6-yl)oxy)-N-(4-hydroxy-benzyl)acetamide A042. MS (ESI) m/z: 423.2 [M+1]⁺.

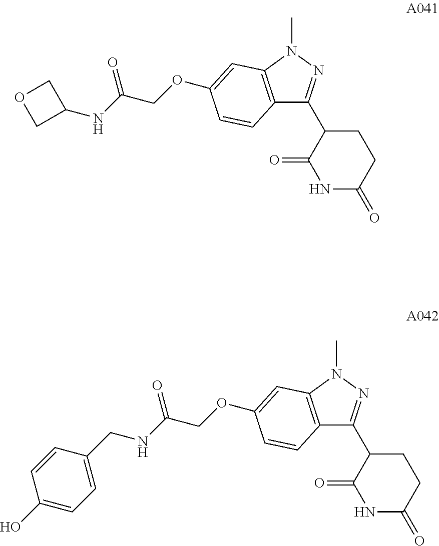 [SMILES: Cn1nc(C2CCC(=O)NC2=O)c2ccc(OCC(=O)NC3COC3)cc21 ; Cn1nc(C2CCC(=O)NC2=O)c2ccc(OCC(=O)NCc3ccc(O)cc3)cc21]

2-((3-(2,6-Dioxopiperidin-3-yl)-1-methyl-1H-indazol-6-yl)oxy)-N-((2-methyl-1H-indol-5-yl)methyl)acetamide A043. MS (ESI) m/z: 460.2 [M+1]⁺.

N-(4-Cyanobicyclo[2.2.2]octan-1-yl)-2-((3-(2,6-dioxopiperidin-3-yl)-1-methyl-1H-indazol-6-yl)oxy)acetamide A044. MS (ESI) m/z: 450.2 [M+1]⁺.

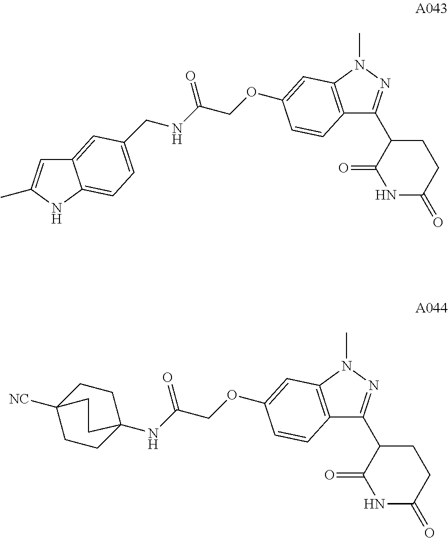 [SMILES: Cc1cc2cc(CNC(=O)COc3ccc4c(C5CCC(=O)NC5=O)nn(C)c4c3)ccc2[nH]1 ; Cn1nc(C2CCC(=O)NC2=O)c2ccc(OCC(=O)NC34CCC(C#N)(CC3)CC4)cc21]

2-((3-(2,6-Dioxopiperidin-3-yl)-1-methyl-1H-indazol-6-yl)oxy)-N-(4-hydroxy-phenethyl)acetamide A045. MS (ESI) m/z: 437.2 [M+1]⁺.

N-((3-Cyclohexyl-1,2,4-oxadiazol-5-yl)methyl)-2-((3-(2,6-dioxopiperidin-3-yl)-1-methyl-1H-indazol-6-yl)oxy)acetamide A046. MS (ESI) m/z: 481.2 [M+1]⁺.

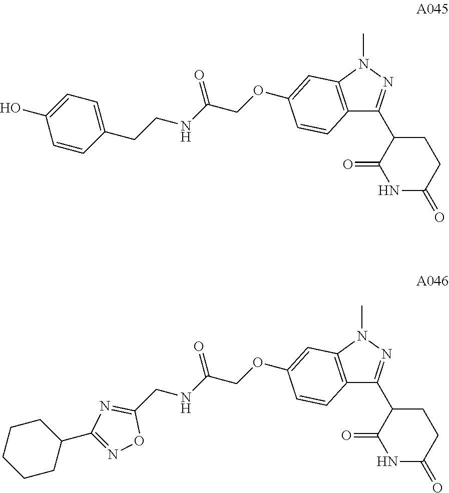 [SMILES: Cn1nc(C2CCC(=O)NC2=O)c2ccc(OCC(=O)NCCc3ccc(O)cc3)cc21 ; Cn1nc(C2CCC(=O)NC2=O)c2ccc(OCC(=O)NCc3nc(C4CCCCC4)no3)cc21]

N-(3,5-Dimethoxybenzyl)-2-((3-(2,6-dioxopiperidin-3-yl)-1-methyl-1H-indazol-6-yl)oxy)acetamide A047. MS (ESI) m/z: 467.2 [M+1]⁺.

2-((3-(2,6-Dioxopiperidin-3-yl)-1-methyl-1H-indazol-6-yl)oxy)-N-(4-isopropyl-benzyl)acetamide A048. MS (ESI) m/z: 449.2 [M+1]⁺.

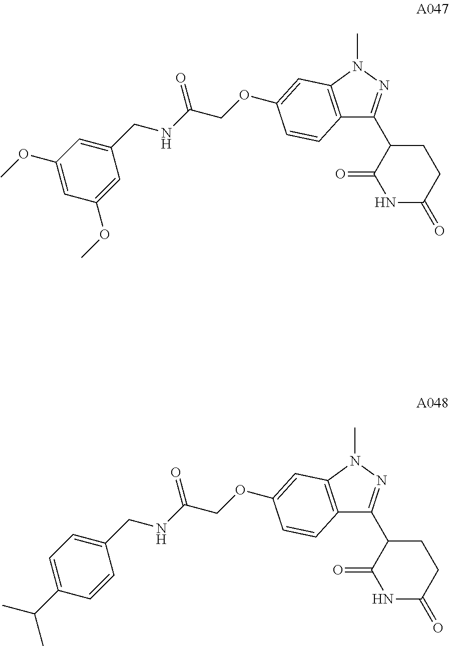 [SMILES: COc1cc(CNC(=O)COc2ccc3c(C4CCC(=O)NC4=O)nn(C)c3c2)cc(OC)c1 ; CC(C)c1ccc(CNC(=O)COc2ccc3c(C4CCC(=O)NC4=O)nn(C)c3c2)cc1]

2-((3-(2,6-Dioxopiperidin-3-yl)-1-methyl-1H-indazol-6-yl)oxy)-N-((3-hydroxy-cyclopentyl)methyl)acetamide A049. MS (ESI) m/z: 415.2 [M+1]⁺.

N-(2,5-Difluorobenzyl)-2-((3-(2,6-dioxopiperidin-3-yl)-1-methyl-1H-indazol-6-yl)oxy)acetamide A050. MS (ESI) m/z: 443.2 [M+1]⁺.

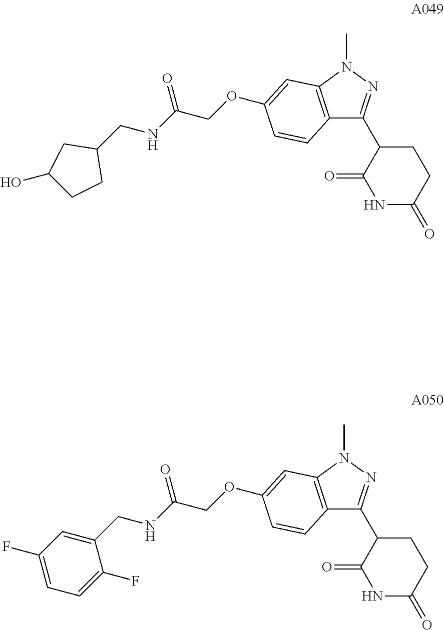 [SMILES: Cn1nc(C2CCC(=O)NC2=O)c2ccc(OCC(=O)NCC3CCC(O)C3)cc21 ; Cn1nc(C2CCC(=O)NC2=O)c2ccc(OCC(=O)NCc3cc(F)ccc3F)cc21]

2-((3-(2,6-Dioxopiperidin-3-yl)-1-methyl-1H-indazol-6-yl)oxy)-N-((1-methyl-1H-indol-3-yl)methyl)acetamide A051. MS (ESI) m/z: 460.2 [M+1]⁺.

2-((3-(2,6-Dioxopiperidin-3-yl)-1-methyl-1H-indazol-6-yl)oxy)-N-neopentyl-acetamide A052. MS (ESI) m/z: 387.3 [M+1]⁺.

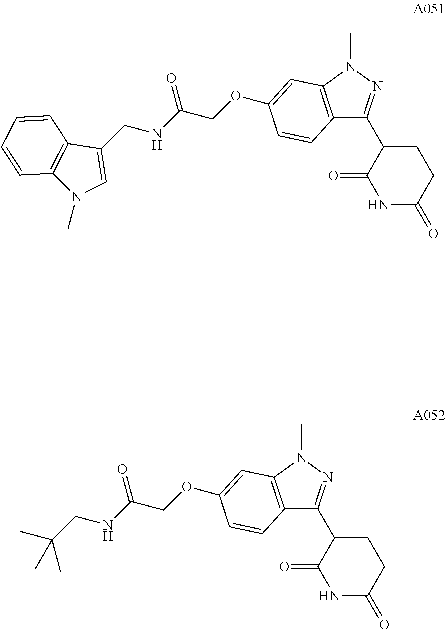 [SMILES: Cn1cc(CNC(=O)COc2ccc3c(C4CCC(=O)NC4=O)nn(C)c3c2)c2ccccc21 ; Cn1nc(C2CCC(=O)NC2=O)c2ccc(OCC(=O)NCC(C)(C)C)cc21]

N-(3,5-Dichlorobenzyl)-2-((3-(2,6-dioxopiperidin-3-yl)-1-methyl-1H-indazol-6-yl)oxy)acetamide A053. MS (ESI) m/z: 475.1 [M+1]⁺.

N-(1,1-Dioxidotetrahydro-2H-thiopyran-4-yl)-2-((3-(2,6-dioxopiperidin-3-yl)-1-methyl-1H-indazol-6-yl)oxy)acetamide A054. MS (ESI) m/z: 449.2 [M+1]⁺.

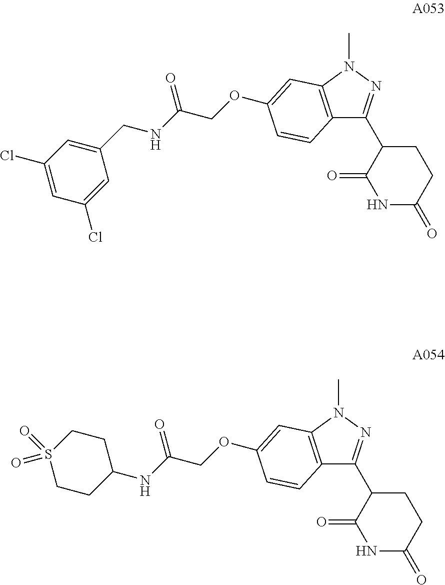 [SMILES: Cn1nc(C2CCC(=O)NC2=O)c2ccc(OCC(=O)NCc3cc(Cl)cc(Cl)c3)cc21 ; Cn1nc(C2CCC(=O)NC2=O)c2ccc(OCC(=O)NC3CCS(=O)(=O)CC3)cc21]

2-((3-(2,6-Dioxopiperidin-3-yl)-1-methyl-1H-indazol-6-yl)oxy)-N-(4-methyl-tetrahydro-2H-pyran-4-yl)acetamide A055. MS (ESI) m/z: 415.2 [M+1]⁺.

N-(2,4-Difluorobenzyl)-2-((3-(2,6-dioxopiperidin-3-yl)-1-methyl-1H-indazol-6-yl)oxy)acetamide A056. MS (ESI) m/z: 443.2 [M+1]⁺.

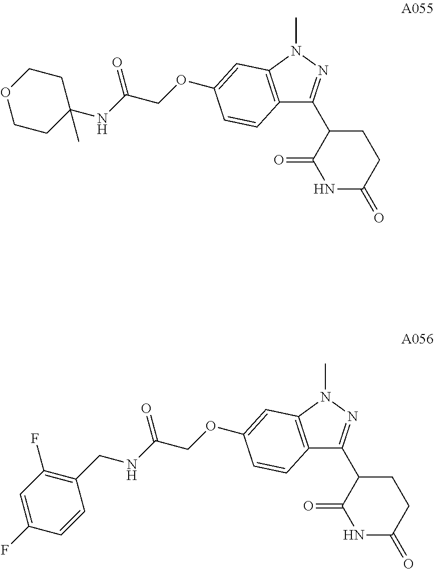 [SMILES: Cn1nc(C2CCC(=O)NC2=O)c2ccc(OCC(=O)NC3(C)CCOCC3)cc21 ; Cn1nc(C2CCC(=O)NC2=O)c2ccc(OCC(=O)NCc3ccc(F)cc3F)cc21]

N-(2-(Benzyloxy)ethyl)-2-((3-(2,6-dioxopiperidin-3-yl)-1-methyl-1H-indazol-6-yl)oxy)acetamide A057. MS (ESI) m/z: 451.2 [M+1]⁺.

N-(1,1-Dioxidothietan-3-yl)-2-((3-(2,6-dioxopiperidin-3-yl)-1-methyl-1H-indazol-6-yl)oxy)acetamide A058. MS (ESI) m/z: 421.1 [M+1]⁺.

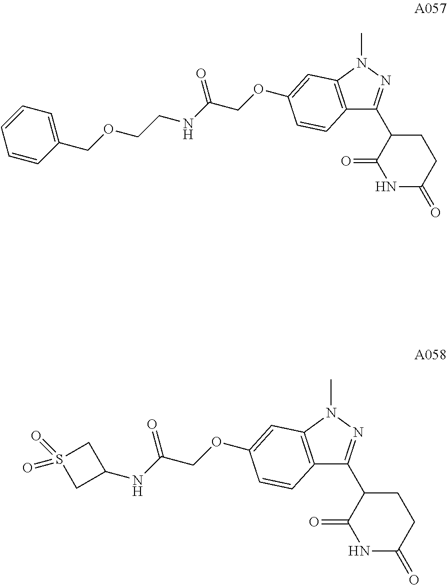 [SMILES: Cn1nc(C2CCC(=O)NC2=O)c2ccc(OCC(=O)NCCOCc3ccccc3)cc21 ; Cn1nc(C2CCC(=O)NC2=O)c2ccc(OCC(=O)NC3CS(=O)(=O)C3)cc21]

2-((3-(2,6-Dioxopiperidin-3-yl)-1-methyl-1H-indazol-6-yl)oxy)-N-(1-methyl-piperidin-4-yl)acetamide A059. MS (ESI) m/z: 414.2 [M+1]⁺.

N-(3-Cyanobenzyl)-2-((3-(2,6-dioxopiperidin-3-yl)-1-methyl-1H-indazol-6-yl)-oxy)acetamide A060. MS (ESI) m/z: 432.2 [M+1]⁺.

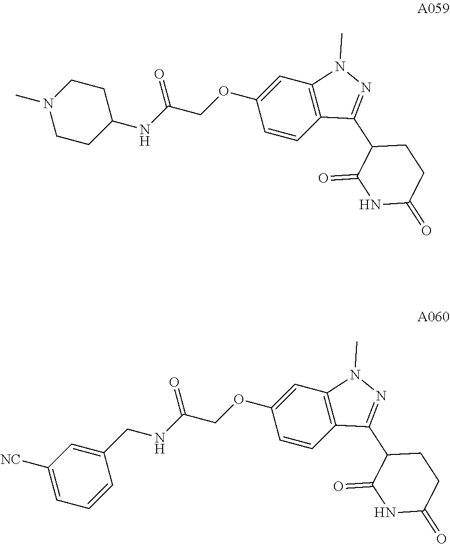 [SMILES: CN1CCC(NC(=O)COc2ccc3c(C4CCC(=O)NC4=O)nn(C)c3c2)CC1 ; Cn1nc(C2CCC(=O)NC2=O)c2ccc(OCC(=O)NCc3cccc(C#N)c3)cc21]

2-((3-(2,6-Dioxopiperidin-3-yl)-1-methyl-1H-indazol-6-yl)oxy)-N-(3-hydroxy-cyclopentyl)acetamide A061. MS (ESI) m/z: 401.2 [M+1]⁺.

N-(2,3-Dimethyltetrahydrofuran-3-yl)-2-((3-(2,6-dioxopiperidin-3-yl)-1-methyl-1H-indazol-6-yl)oxy)acetamide A062. MS (ESI) m/z: 415.2 [M+1]⁺.

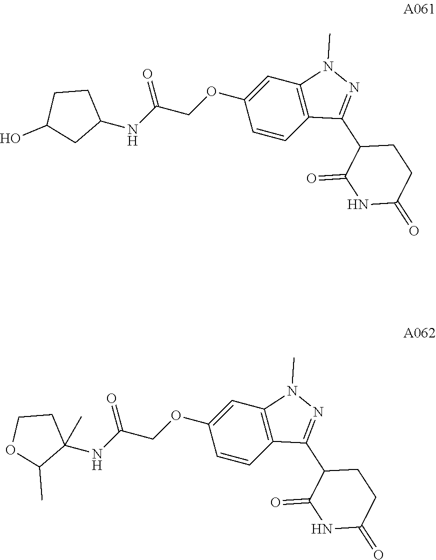 [SMILES: Cn1nc(C2CCC(=O)NC2=O)c2ccc(OCC(=O)NC3CCC(O)C3)cc21 ; CC1OCCC1(C)NC(=O)COc1ccc2c(C3CCC(=O)NC3=O)nn(C)c2c1]

N-((2-Chlorothiazol-5-yl)methyl)-2-((3-(2,6-dioxopiperidin-3-yl)-1-methyl-1H-indazol-6-yl)oxy)acetamide A063. MS (ESI) m/z: 448.1 [M+1]⁺.

2-((3-(2,6-Dioxopiperidin-3-yl)-1-methyl-1H-indazol-6-yl)oxy)-N—((R)-1-(5-methylfuran-2-yl) propyl)acetamide A064. MS (ESI) m/z: 439.2 [M+1]⁺.

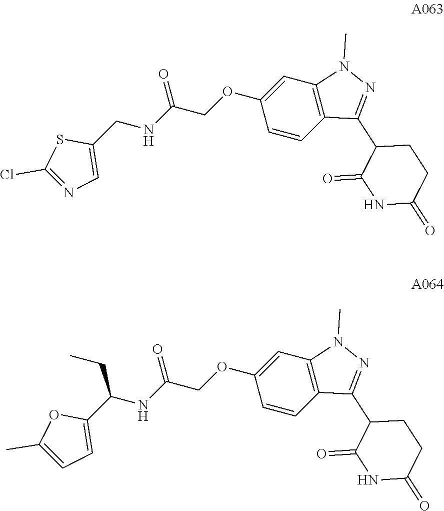 [SMILES: Cn1nc(C2CCC(=O)NC2=O)c2ccc(OCC(=O)NCc3cnc(Cl)s3)cc21 ; CC[C@@H](NC(=O)COc1ccc2c(C3CCC(=O)NC3=O)nn(C)c2c1)c1ccc(C)o1]

2-((3-(2,6-Dioxopiperidin-3-yl)-1-methyl-1H-indazol-6-yl)oxy)-N-(pyridin-2-yl)-acetamide A065. MS (ESI) m/z: 394.2 [M+1]⁺.

2-((3-(2,6-Dioxopiperidin-3-yl)-1-methyl-1H-indazol-6-yl)oxy)-N—((R)-2-oxo-pyrrolidin-3-yl)acetamide A066. MS (ESI) m/z: 400.2 [M+1]⁺.

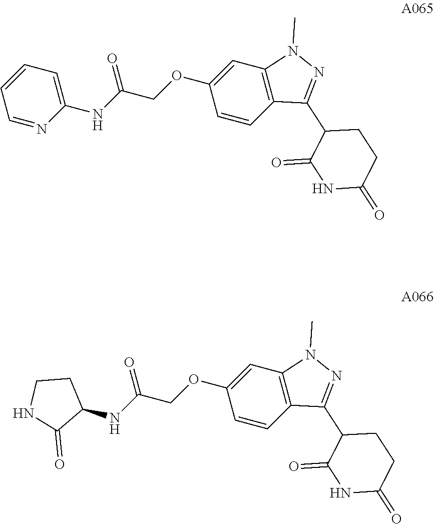 [SMILES: Cn1nc(C2CCC(=O)NC2=O)c2ccc(OCC(=O)Nc3ccccn3)cc21 ; Cn1nc(C2CCC(=O)NC2=O)c2ccc(OCC(=O)N[C@@H]3CCNC3=O)cc21]

2-((3-(2,6-Dioxopiperidin-3-yl)-1-methyl-1H-indazol-6-yl)oxy)-N-((5-phenyl-1,2,4-oxadiazol-3-yl)methyl)acetamide A067. MS (ESI) m/z: 475.2 [M+1]⁺.

N-(Cyclopentylmethyl)-2-((3-(2,6-dioxopiperidin-3-yl)-1-methyl-1H-indazol-6-yl)oxy)acetamide A068. MS (ESI) m/z: 399.2 [M+1]⁺.

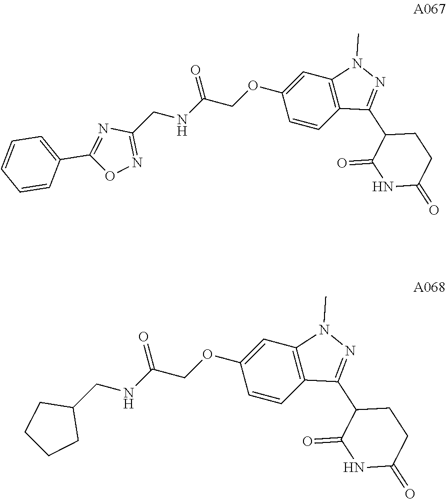 [SMILES: Cn1nc(C2CCC(=O)NC2=O)c2ccc(OCC(=O)NCc3noc(-c4ccccc4)n3)cc21 ; Cn1nc(C2CCC(=O)NC2=O)c2ccc(OCC(=O)NCC3CCCC3)cc21]

N-(2-(Cyclopentyloxy)ethyl)-2-((3-(2,6-dioxopiperidin-3-yl)-1-methyl-1H-indazol-6-yl)oxy)acetamide A069. MS (ESI) m/z: 429.2 [M+1]⁺.

N-(2-(3,5-Dimethyl-1H-1,2,4-triazol-1-yl)ethyl)-2-((3-(2,6-dioxopiperidin-3-yl)-1-methyl-1H-indazol-6-yl)oxy)acetamide A071. MS (ESI) m/z: 440.2 [M+1]⁺.

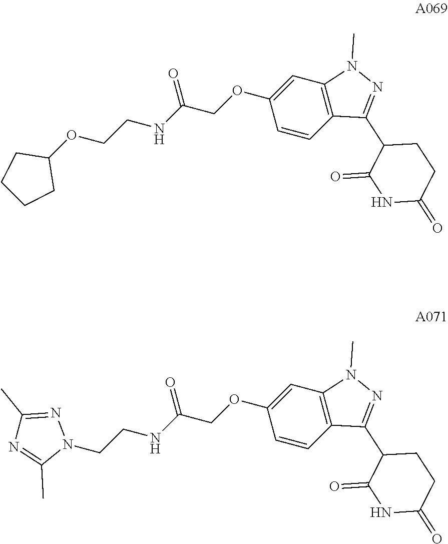 [SMILES: Cn1nc(C2CCC(=O)NC2=O)c2ccc(OCC(=O)NCCOC3CCCC3)cc21 ; Cc1nc(C)n(CCNC(=O)COc2ccc3c(C4CCC(=O)NC4=O)nn(C)c3c2)n1]

2-((3-(2,6-Dioxopiperidin-3-yl)-1-methyl-1H-indazol-6-yl)oxy)-N-(tetrahydro-2H-pyran-4-yl)acetamide A072. MS (ESI) m/z: 401.2 [M+1]⁺.

N-Cyclobutyl-2-((3-(2,6-dioxopiperidin-3-yl)-1-methyl-1H-indazol-6-yl)oxy)-acetamide A073. MS (ESI) m/z: 371.2 [M+1]⁺.

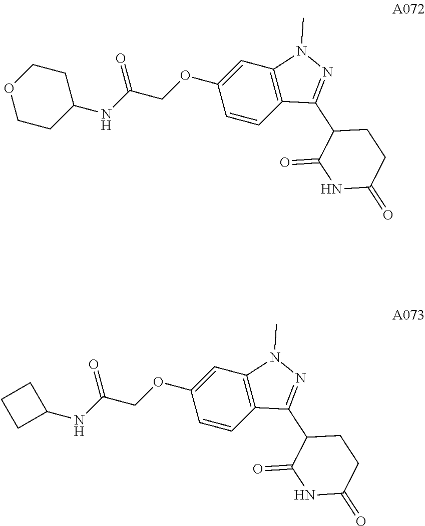 [SMILES: Cn1nc(C2CCC(=O)NC2=O)c2ccc(OCC(=O)NC3CCOCC3)cc21 ; Cn1nc(C2CCC(=O)NC2=O)c2ccc(OCC(=O)NC3CCC3)cc21]

N—((S)-1-(4-Chlorophenyl)ethyl)-2-((3-(2,6-dioxopiperidin-3-yl)-1-methyl-1H-indazol-6-yl)oxy)acetamide A074. MS (ESI) m/z: 455.1 [M+1]⁺.

2-((3-(2,6-Dioxopiperidin-3-yl)-1-methyl-1H-indazol-6-yl)oxy)-N-(1-isopropyl-1H-pyrazol-4-yl)acetamide A075. MS (ESI) m/z: 425.2 [M+1]⁺.

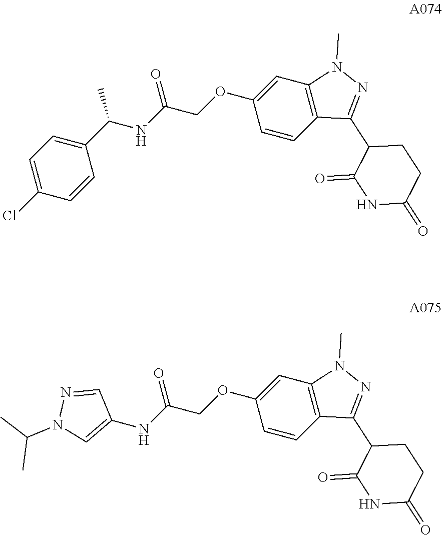 [SMILES: C[C@H](NC(=O)COc1ccc2c(C3CCC(=O)NC3=O)nn(C)c2c1)c1ccc(Cl)cc1 ; CC(C)n1cc(NC(=O)COc2ccc3c(C4CCC(=O)NC4=O)nn(C)c3c2)cn1]

2-((3-(2,6-Dioxopiperidin-3-yl)-1-methyl-1H-indazol-6-yl)oxy)-N-(1-(tetrahydro-2H-pyran-4-yl)-1H-pyrazol-4-yl)acetamide A076. MS (ESI) m/z: 467.2 [M+1]⁺.

2-((3-(2,6-Dioxopiperidin-3-yl)-1-methyl-1H-indazol-6-yl)oxy)-N-((1-methyl-2-oxo-1,2-dihydropyridin-4-yl)methyl)acetamide A077. MS (ESI) m/z: 438.1 [M+1]⁺.

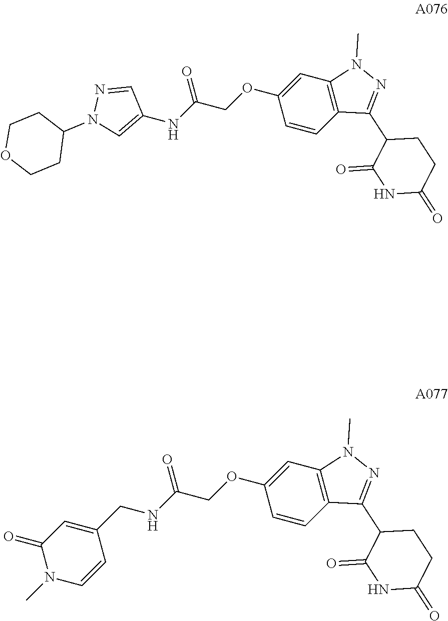 [SMILES: Cn1nc(C2CCC(=O)NC2=O)c2ccc(OCC(=O)Nc3cnn(C4CCOCC4)c3)cc21 ; Cn1ccc(CNC(=O)COc2ccc3c(C4CCC(=O)NC4=O)nn(C)c3c2)cc1=O]

2-((3-(2,6-Dioxopiperidin-3-yl)-1-methyl-1H-indazol-6-yl)oxy)-N-(4-(2-methyl-1H-imidazol-1-yl)phenyl)acetamide A078. MS (ESI) m/z: 473.2 [M+1]⁺.

2-((3-(2,6-Dioxopiperidin-3-yl)-1-methyl-1H-indazol-6-yl)oxy)-N-(3-fluoro-bicyclo[1.1.1]pentan-1-yl)acetamide A079. MS (ESI) m/z: 401.1 [M+1]⁺.

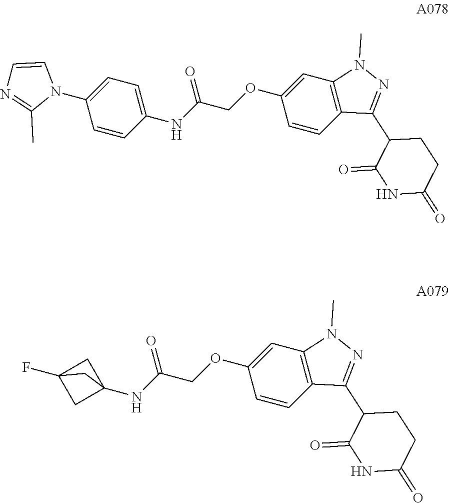 [SMILES: Cc1nccn1-c1ccc(NC(=O)COc2ccc3c(C4CCC(=O)NC4=O)nn(C)c3c2)cc1 ; Cn1nc(C2CCC(=O)NC2=O)c2ccc(OCC(=O)NC34CC(F)(C3)C4)cc21]

N-(4-(1H-Imidazol-2-yl)phenyl)-2-((3-(2,6-dioxopiperidin-3-yl)-1-methyl-1H-indazol-6-yl)oxy)acetamide A080. MS (ESI) m/z: 459.1 [M+1]⁺.

2-((3-(2,6-Dioxopiperidin-3-yl)-1-methyl-1H-indazol-6-yl)oxy)-N-(4-(4-methyl-piperazin-1-yl)phenyl)acetamide A081. MS (ESI) m/z: 491.1 [M+1]⁺.

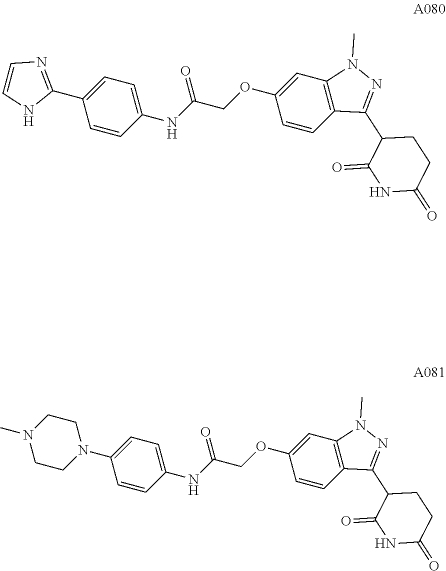 [SMILES: Cn1nc(C2CCC(=O)NC2=O)c2ccc(OCC(=O)Nc3ccc(-c4ncc[nH]4)cc3)cc21 ; CN1CCN(c2ccc(NC(=O)COc3ccc4c(C5CCC(=O)NC5=O)nn(C)c4c3)cc2)CC1]

2-((3-(2,6-Dioxopiperidin-3-yl)-1-methyl-1H-indazol-6-yl)oxy)-N-(4-(oxazol-5-yl)phenyl)acetamide A082. MS (ESI) m/z: 460.1 [M+1]⁺.

2-((3-(2,6-Dioxopiperidin-3-yl)-1-methyl-1H-indazol-6-yl)oxy)-N-(1-methyl-1H-pyrazol-5-yl)acetamide A083. MS (ESI) m/z: 397.1 [M+1]⁺.

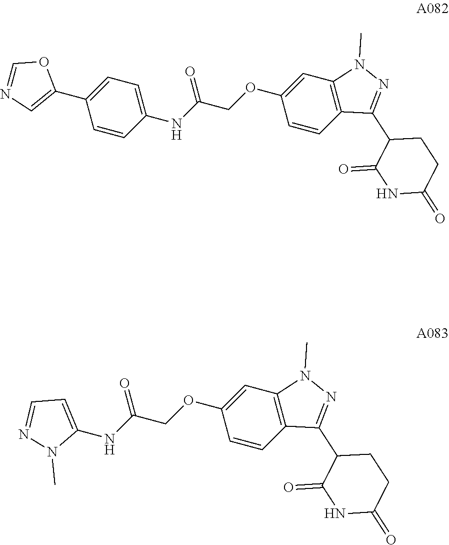 [SMILES: Cn1nc(C2CCC(=O)NC2=O)c2ccc(OCC(=O)Nc3ccc(-c4cnco4)cc3)cc21 ; Cn1nccc1NC(=O)COc1ccc2c(C3CCC(=O)NC3=O)nn(C)c2c1]

2-((3-(2,6-Dioxopiperidin-3-yl)-1-methyl-1H-indazol-6-yl)oxy)-N-(4-(p-tolyl-oxy)phenyl)acetamide A084. MS (ESI) m/z: 499.2 [M+1]⁺.

N-(3-(Tert-butyl)phenyl)-2-((3-(2,6-dioxopiperidin-3-yl)-1-methyl-1H-indazol-6-yl)oxy)acetamide A085. MS (ESI) m/z: 449.2 [M+1]⁺.

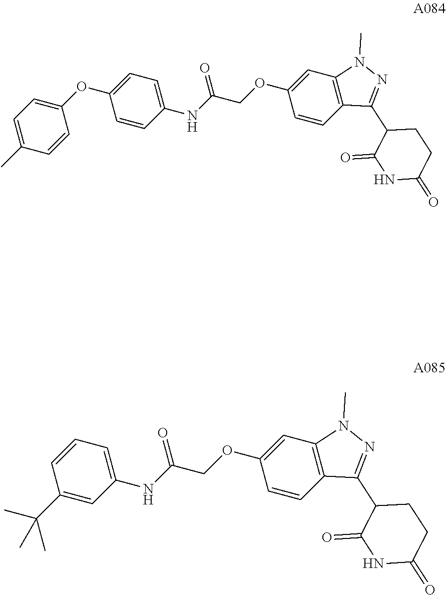 [SMILES: Cc1ccc(Oc2ccc(NC(=O)COc3ccc4c(C5CCC(=O)NC5=O)nn(C)c4c3)cc2)cc1 ; Cn1nc(C2CCC(=O)NC2=O)c2ccc(OCC(=O)Nc3cccc(C(C)(C)C)c3)cc21]

2-((3-(2,6-Dioxopiperidin-3-yl)-1-methyl-1H-indazol-6-yl)oxy)-N-(1-(oxetan-3-yl)-1H-pyrazol-4-yl)acetamide A086. MS (ESI) m/z: 439.1 [M+1]⁺.

N-(4-(1H-Imidazol-5-yl)phenyl)-2-((3-(2,6-dioxopiperidin-3-yl)-1-methyl-1H-indazol-6-yl)oxy)acetamide A087. MS (ESI) m/z: 459.1 [M+1]⁺.

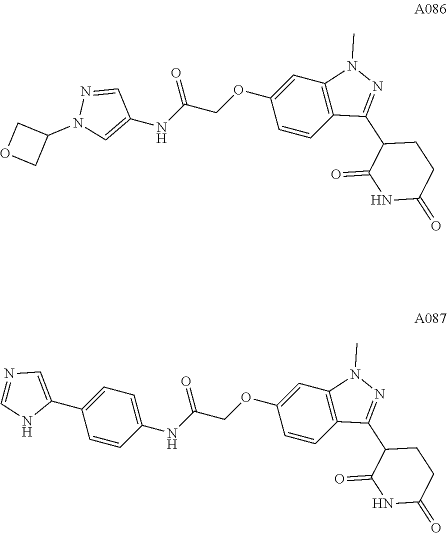 [SMILES: Cn1nc(C2CCC(=O)NC2=O)c2ccc(OCC(=O)Nc3cnn(C4COC4)c3)cc21 ; Cn1nc(C2CCC(=O)NC2=O)c2ccc(OCC(=O)Nc3ccc(-c4cnc[nH]4)cc3)cc21]

2-((3-(2,6-Dioxopiperidin-3-yl)-1-methyl-1H-indazol-6-yl)oxy)-N-(4-(trifluoro-methoxy)phenyl)acetamide A088. MS (ESI) m/z: 477.1 [M+1]⁺.

2-((3-(2,6-Dioxopiperidin-3-yl)-1-methyl-1H-indazol-6-yl)oxy)-N-(3-isopropoxy-phenyl)acetamide A089. MS (ESI) m/z: 451.2 [M+1]⁺.

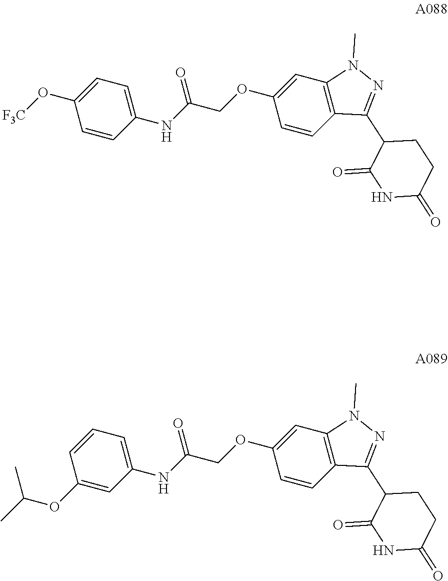 [SMILES: Cn1nc(C2CCC(=O)NC2=O)c2ccc(OCC(=O)Nc3ccc(OC(F)(F)F)cc3)cc21 ; CC(C)Oc1cccc(NC(=O)COc2ccc3c(C4CCC(=O)NC4=O)nn(C)c3c2)c1]

N-(5-Cyclopropyl-1H-pyrazol-3-yl)-2-((3-(2,6-dioxopiperidin-3-yl)-1-methyl-1H-indazol-6-yl)oxy)acetamide A090. MS (ESI) m/z: 423.2 [M+1]⁺.

N-(3-(1H-Imidazol-1-yl)phenyl)-2-((3-(2,6-dioxopiperidin-3-yl)-1-methyl-1H-indazol-6-yl)oxy)acetamide A091. MS (ESI) m/z: 459.2 [M+1]⁺.

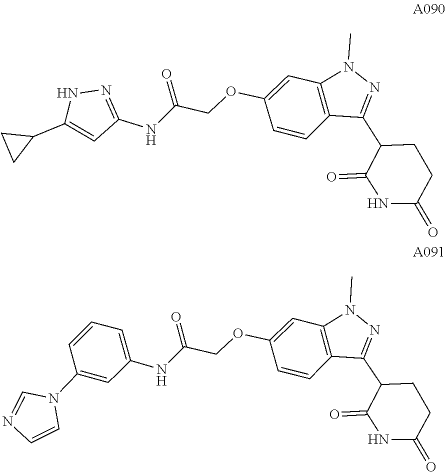 [SMILES: Cn1nc(C2CCC(=O)NC2=O)c2ccc(OCC(=O)Nc3cc(C4CC4)[nH]n3)cc21 ; Cn1nc(C2CCC(=O)NC2=O)c2ccc(OCC(=O)Nc3cccc(-n4ccnc4)c3)cc21]

N-(Cyclopropylmethyl)-2-((3-(2,6-dioxopiperidin-3-yl)-1-methyl-1H-indazol-6-yl)oxy)acetamide A092. MS (ESI) m/z: 371.2 [M+1]⁺.

N-Cyclohexyl-2-((3-(2,6-dioxopiperidin-3-yl)-1-methyl-1H-indazol-6-yl)oxy)-acetamide A093. MS (ESI) m/z: 399.2 [M+1]⁺.

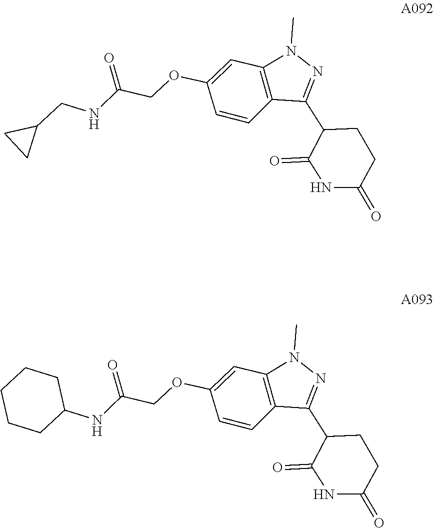 [SMILES: Cn1nc(C2CCC(=O)NC2=O)c2ccc(OCC(=O)NCC3CC3)cc21 ; Cn1nc(C2CCC(=O)NC2=O)c2ccc(OCC(=O)NC3CCCCC3)cc21]

N-Cyclopentyl-2-((3-(2,6-dioxopiperidin-3-yl)-1-methyl-1H-indazol-6-yl)oxy)-acetamide A094. MS (ESI) m/z: 385.2 [M+1]⁺.

2-((3-(2,6-Dioxopiperidin-3-yl)-1-methyl-1H-indazol-6-yl)oxy)-N-(thiophen-2-ylmethyl)acetamide A095. MS (ESI) m/z: 413.1 [M+1]⁺.

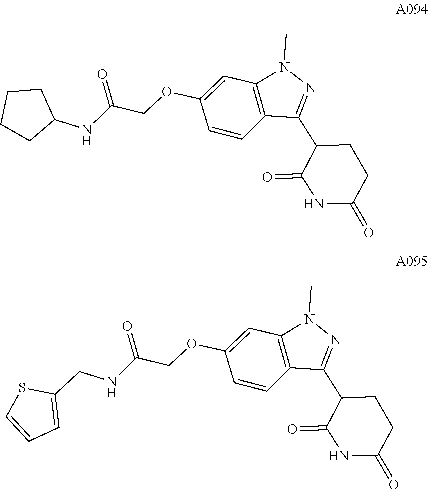 [SMILES: Cn1nc(C2CCC(=O)NC2=O)c2ccc(OCC(=O)NC3CCCC3)cc21 ; Cn1nc(C2CCC(=O)NC2=O)c2ccc(OCC(=O)NCc3cccs3)cc21]

2-((3-(2,6-Dioxopiperidin-3-yl)-1-methyl-1H-indazol-6-yl)oxy)-N-(4-(morpholinomethyl)benzyl)acetamide A096. MS (ESI) m/z: 506.2 [M+1]⁺.

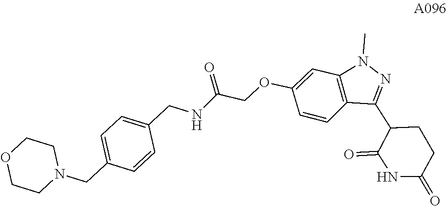 [SMILES: Cn1nc(C2CCC(=O)NC2=O)c2ccc(OCC(=O)NCc3ccc(CN4CCOCC4)cc3)cc21]

N-(3-Cyano-4,5-dimethyl-1H-pyrrol-2-yl)-2-((3-(2,6-dioxopiperidin-3-yl)-1-methyl-1H-indazol-7-yl)oxy)acetamide B001. MS (ESI) m/z: 435.2 [M+1]⁺.

2-((3-(2,6-Dioxopiperidin-3-yl)-1-methyl-1H-indazol-7-yl)oxy)-N-(4-(trifluoro-methoxy)phenyl)acetamide B002. MS (ESI) m/z: 477.1 [M+1]⁺.

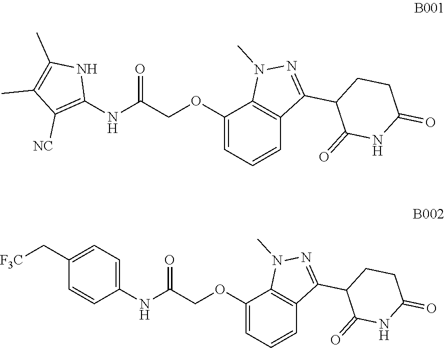 [SMILES: Cc1[nH]c(NC(=O)COc2cccc3c(C4CCC(=O)NC4=O)nn(C)c23)c(C#N)c1C ; Cn1nc(C2CCC(=O)NC2=O)c2cccc(OCC(=O)Nc3ccc(CC(F)(F)F)cc3)c21]

6-(Tert-butyl)₃-ethyl 2-(2-((3-(2,6-dioxopiperidin-3-yl)-1-methyl-1H-indazol-7-yl)oxy)acetamido)-4,7-dihydrothieno[2,3-c]pyridine-3,6 (5H)-dicarboxylate B003.

Diethyl 3-(2-((3-(2,6-dioxopiperidin-3-yl)-1-methyl-1H-indazol-7-yl)oxy)-acetamido)-1H-pyrrole-2,4-dicarboxylate B004. MS (ESI) m/z: 526.2 [M+1]⁺.

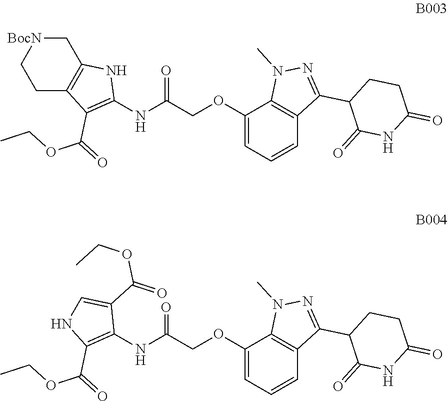 [SMILES: CCOC(=O)c1c(NC(=O)COc2cccc3c(C4CCC(=O)NC4=O)nn(C)c23)[nH]c2c1CCN(C(=O)OC(C)(C)C)C2 ; CCOC(=O)c1c[nH]c(C(=O)OCC)c1NC(=O)COc1cccc2c(C3CCC(=O)NC3=O)nn(C)c12]

Ethyl 2-(2-((3-(2,6-dioxopiperidin-3-yl)-1-methyl-1H-indazol-7-yl)oxy)-acetamido)-4-methyl-4,5,6,7-tetrahydrobenzo[b]thiophene-3-carboxylate B005. MS (ESI) m/z: 539.1 [M+1]⁺.

N-(3-Cyano-1H-pyrrol-2-yl)-2-((3-(2,6-dioxopiperidin-3-yl)-1-methyl-1H-indazol-7-yl)oxy)acetamide B006. MS (ESI) m/z: 407.1 [M+1]⁺.

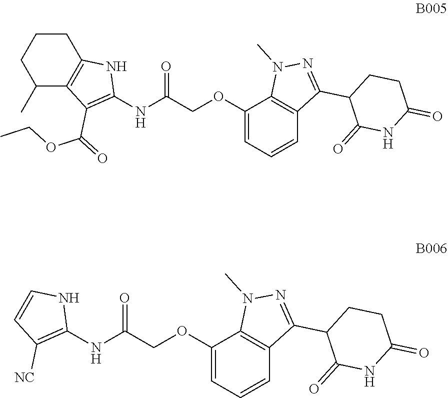 [SMILES: CCOC(=O)c1c(NC(=O)COc2cccc3c(C4CCC(=O)NC4=O)nn(C)c23)[nH]c2c1C(C)CCC2 ; Cn1nc(C2CCC(=O)NC2=O)c2cccc(OCC(=O)Nc3[nH]ccc3C#N)c21]

Ethyl 5-(2-((3-(2,6-dioxopiperidin-3-yl)-1-methyl-1H-indazol-7-yl)oxy)-acetamido)-3-methyl-1H-pyrazole-4-carboxylate B007. MS (ESI) m/z: 469.2 [M+1]⁺.

2-((3-(2,6-Dioxopiperidin-3-yl)-1-methyl-1H-indazol-7-yl)oxy)-N-(3-(pyridin-2-yl)-1H-pyrazol-4-yl)acetamide B008. MS (ESI) m/z: 460.2 [M+1]⁺.

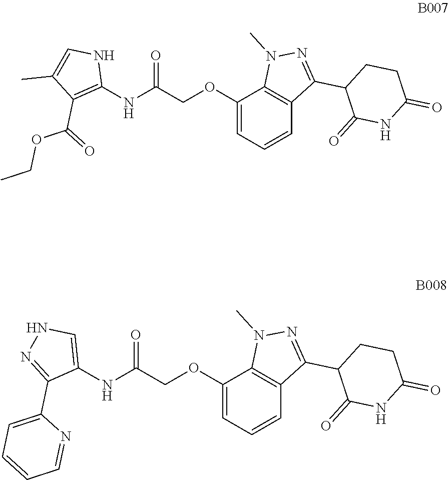 [SMILES: CCOC(=O)c1c(C)c[nH]c1NC(=O)COc1cccc2c(C3CCC(=O)NC3=O)nn(C)c12 ; Cn1nc(C2CCC(=O)NC2=O)c2cccc(OCC(=O)Nc3c[nH]nc3-c3ccccn3)c21]

Diethyl 2-(2-((3-(2,6-dioxopiperidin-3-yl)-1-methyl-1H-indazol-7-yl)oxy)-acetamido)thiophene-3,4-dicarboxylate B009. MS (ESI) m/z: 543.1 [M+1]⁺.

N-(5-Chloro-2-cyanophenyl)-2-((3-(2,6-dioxopiperidin-3-yl)-1-methyl-1H-indazol-7-yl)oxy)acetamide B010. MS (ESI) m/z: 452.1 [M+1]⁺.

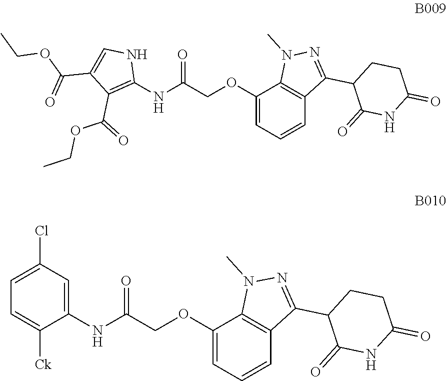

2-((3-(2,6-Dioxopiperidin-3-yl)-1-methyl-1H-indazol-7-yl)oxy)-N-(4-(oxazol-5-yl)phenyl)acetamide B011. MS (ESI) m/z: 460.1 [M+1]⁺.

N-(4-Cyano-3-methylisothiazol-5-yl)-2-((3-(2,6-dioxopiperidin-3-yl)-1-methyl-1H-indazol-7-yl)oxy)acetamide B012. MS (ESI) m/z: 439.1 [M+1]⁺.

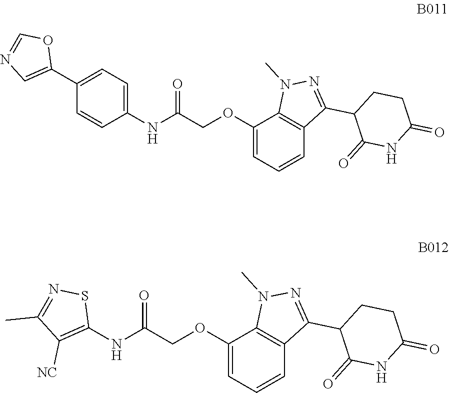 [SMILES: Cn1nc(C2CCC(=O)NC2=O)c2cccc(OCC(=O)Nc3ccc(-c4cnco4)cc3)c21 ; Cc1nsc(NC(=O)COc2cccc3c(C4CCC(=O)NC4=O)nn(C)c23)c1C#N]

N-(2-Cyano-3-methoxyphenyl)-2-((3-(2,6-dioxopiperidin-3-yl)-1-methyl-1H-indazol-7-yl)oxy)acetamide B013. MS (ESI) m/z: 448.2 [M+1]⁺.

N-(4-Cyano-1-phenyl-1H-pyrazol-5-yl)-2-((3-(2,6-dioxopiperidin-3-yl)-1-methyl-1H-indazol-7-yl)oxy)acetamide B014. MS (ESI) m/z: 484.1 [M+1]⁺.

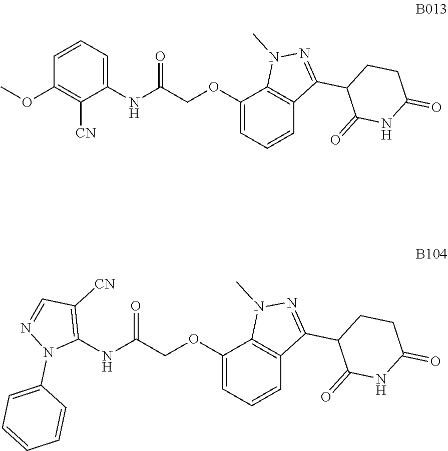 [SMILES: COc1cccc(NC(=O)COc2cccc3c(C4CCC(=O)NC4=O)nn(C)c23)c1C#N ; Cn1nc(C2CCC(=O)NC2=O)c2cccc(OCC(=O)Nc3c(C#N)cnn3-c3ccccc3)c21]

N-(3-Cyano-6-methyl-4,5,6,7-tetrahydrobenzo[b]thiophen-2-yl)-2-((3-(2,6-dioxo-piperidin-3-yl)-1-methyl-1H-indazol-7-yl)oxy)acetamide B015. MS (ESI) m/z: 492.1 [M+1]⁺.

N-(2-Cyano-5-(trifluoromethyl)phenyl)-2-((3-(2,6-dioxopiperidin-3-yl)-1-methyl-1H-indazol-7-yl)oxy)acetamide B016. MS (ESI) m/z: 486.1 [M+1]⁺.

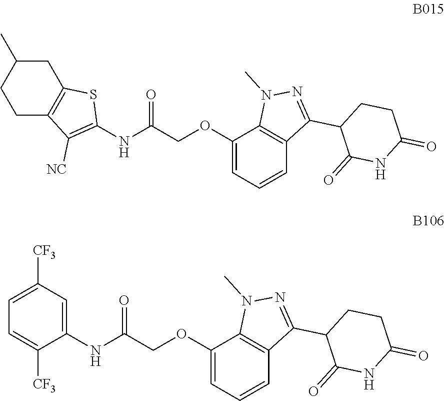 [SMILES: CC1CCc2c(sc(NC(=O)COc3cccc4c(C5CCC(=O)NC5=O)nn(C)c34)c2C#N)C1 ; Cn1nc(C2CCC(=O)NC2=O)c2cccc(OCC(=O)Nc3cc(C(F)(F)F)ccc3C(F)(F)F)c21]

N-(2-Cyano-3-methylphenyl)-2-((3-(2,6-dioxopiperidin-3-yl)-1-methyl-1H-indazol-7-yl)oxy)acetamide B017. MS (ESI) m/z: 432.2 [M+1]⁺.

N-(2-Cyano-4-methoxyphenyl)-2-((3-(2,6-dioxopiperidin-3-yl)-1-methyl-1H-indazol-7-yl)oxy)acetamide B018. MS (ESI) m/z: 448.2 [M+1]⁺.

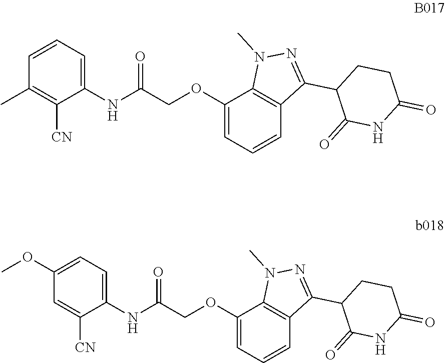 [SMILES: Cc1cccc(NC(=O)COc2cccc3c(C4CCC(=O)NC4=O)nn(C)c23)c1C#N ; COc1ccc(NC(=O)COc2cccc3c(C4CCC(=O)NC4=O)nn(C)c23)c(C#N)c1]

Methyl 4-(2-((3-(2,6-dioxopiperidin-3-yl)-1-methyl-1H-indazol-7-yl)oxy)-acetamido)-2-methylthiophene-3-carboxylate B019. MS (ESI) m/z: 471.1 [M+1]⁺.

Ethyl 5-(2-((3-(2,6-dioxopiperidin-3-yl)-1-methyl-1H-indazol-7-yl)oxy)-acetamido) thiazole-4-carboxylate B020. MS (ESI) m/z: 472.1 [M+1]⁺.

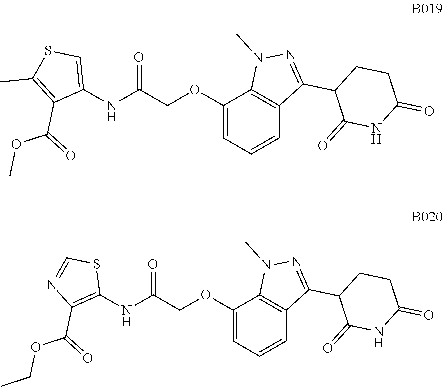 [SMILES: COC(=O)c1c(NC(=O)COc2cccc3c(C4CCC(=O)NC4=O)nn(C)c23)csc1C ; CCOC(=O)c1ncsc1NC(=O)COc1cccc2c(C3CCC(=O)NC3=O)nn(C)c12]

Ethyl 2-(2-((3-(2,6-dioxopiperidin-3-yl)-1-methyl-1H-indazol-7-yl)oxy)-acetamido)-4-isopropylthiophene-3-carboxylate B021. MS (ESI) m/z: 513.2 [M+1]⁺.

N-(3-Cyano-4-methylthiophen-2-yl)-2-((3-(2,6-dioxopiperidin-3-yl)-1-methyl-1H-indazol-7-yl)oxy)acetamide B022. MS (ESI) m/z: 438.1 [M+1]⁺.

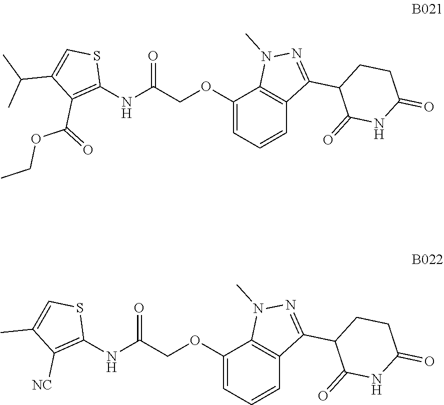 [SMILES: CCOC(=O)c1c(C(C)C)csc1NC(=O)COc1cccc2c(C3CCC(=O)NC3=O)nn(C)c12 ; Cc1csc(NC(=O)COc2cccc3c(C4CCC(=O)NC4=O)nn(C)c23)c1C#N]

N-(3-(2-Chlorobenzoyl)-5-ethylthiophen-2-yl)-2-((3-(2,6-dioxopiperidin-3-yl)-1-methyl-1H-indazol-7-yl)oxy)acetamide B023. MS (ESI) m/z: 565.1 [M+1]⁺.

N-(2-Cyano-6-methylphenyl)-2-((3-(2,6-dioxopiperidin-3-yl)-1-methyl-1H-indazol-7-yl)oxy)acetamide B024. MS (ESI) m/z: 432.2 [M+1]⁺.

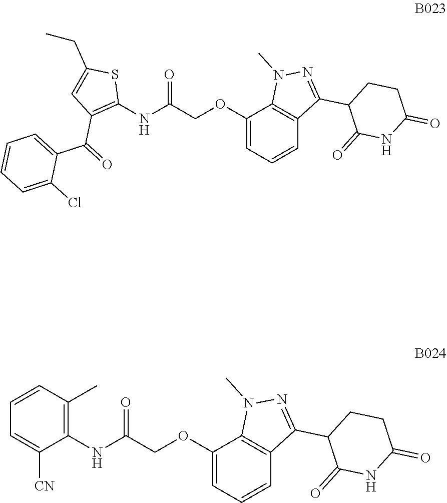 [SMILES: CCc1cc(C(=O)c2ccccc2Cl)c(NC(=O)COc2cccc3c(C4CCC(=O)NC4=O)nn(C)c23)s1 ; Cc1cccc(C#N)c1NC(=O)COc1cccc2c(C3CCC(=O)NC3=O)nn(C)c12]

N-(3-Cyano-4-methyl-1H-pyrrol-2-yl)-2-((3-(2,6-dioxopiperidin-3-yl)-1-methyl-1H-indazol-7-yl)oxy)acetamide B025. MS (ESI) m/z: 421.2 [M+1]⁺.

Ethyl 5-cyano-4-(2-((3-(2,6-dioxopiperidin-3-yl)-1-methyl-1H-indazol-7-yl)oxy)-acetamido)-2-(methylthio)thiophene-3-carboxylate B026. MS (ESI) m/z: 542.1 [M+1]⁺.

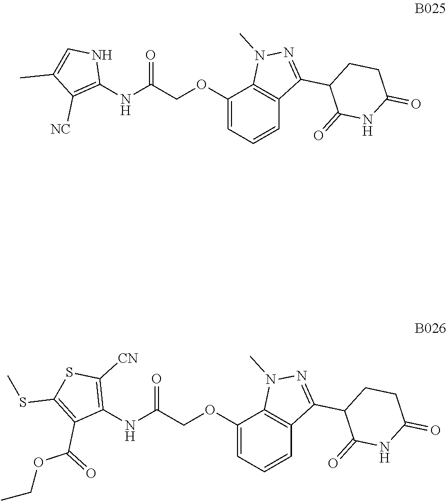 [SMILES: Cc1c[nH]c(NC(=O)COc2cccc3c(C4CCC(=O)NC4=O)nn(C)c23)c1C#N ; CCOC(=O)c1c(SC)sc(C#N)c1NC(=O)COc1cccc2c(C3CCC(=O)NC3=O)nn(C)c12]

N-(5-(Tert-butyl)-1,3,4-thiadiazol-2-yl)-2-((3-(2,6-dioxopiperidin-3-yl)-1-methyl-1H-indazol-7-yl)oxy)acetamide B027. MS (ESI) m/z: 457.2 [M+1]⁺.

Ethyl 2-(2-((3-(2,6-dioxopiperidin-3-yl)-1-methyl-1H-indazol-7-yl)oxy)-acetamido)-4-phenylthiophene-3-carboxylate B028. MS (ESI) m/z: 547.2 [M+1]⁺.

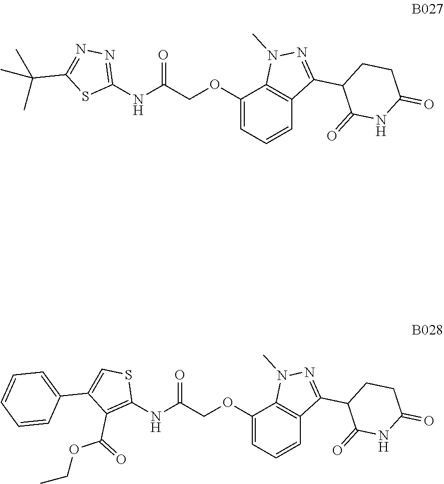 [SMILES: Cn1nc(C2CCC(=O)NC2=O)c2cccc(OCC(=O)Nc3nnc(C(C)(C)C)s3)c21 ; CCOC(=O)c1c(-c2ccccc2)csc1NC(=O)COc1cccc2c(C3CCC(=O)NC3=O)nn(C)c12]

Methyl 2-(2-((3-(2,6-dioxopiperidin-3-yl)-1-methyl-1H-indazol-7-yl)oxy)-acetamido)-1H-indole-3-carboxylate B029. MS (ESI) m/z: 490.2 [M+1]⁺.

2-((3-(2,6-Dioxopiperidin-3-yl)-1-methyl-1H-indazol-7-yl)oxy)-N-(2-hydroxy-2,3-dihydro-1H-inden-1-yl)acetamide B030. MS (ESI) m/z: 449.2 [M+1]⁺.

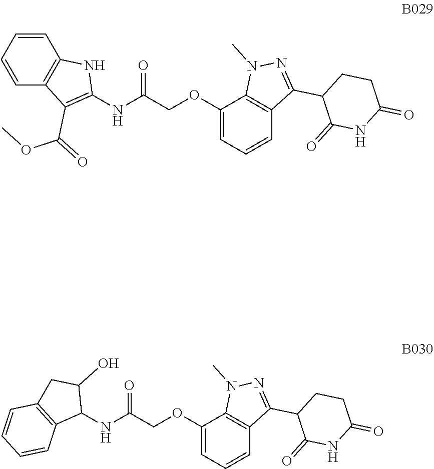 [SMILES: COC(=O)c1c(NC(=O)COc2cccc3c(C4CCC(=O)NC4=O)nn(C)c23)[nH]c2ccccc12 ; Cn1nc(C2CCC(=O)NC2=O)c2cccc(OCC(=O)NC3c4ccccc4CC3O)c21]

N-(4-Cyano-3-methyl-1-phenyl-1H-pyrazol-5-yl)-2-((3-(2,6-dioxopiperidin-3-yl)-1-methyl-1H-indazol-7-yl)oxy)acetamide B031. MS (ESI) m/z: 498.1 [M+1]⁺.

3-Cyano-4-(2-((3-(2,6-dioxopiperidin-3-yl)-1-methyl-1H-indazol-7-yl)oxy)-acetamido)benzoic acid B032. MS (ESI) m/z: 462.2 [M+1]⁺.

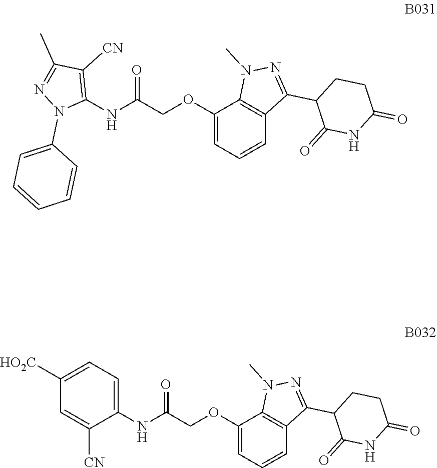 [SMILES: Cc1nn(-c2ccccc2)c(NC(=O)COc2cccc3c(C4CCC(=O)NC4=O)nn(C)c23)c1C#N ; Cn1nc(C2CCC(=O)NC2=O)c2cccc(OCC(=O)Nc3ccc(C(=O)O)cc3C#N)c21]

2-((3-(2,6-Dioxopiperidin-3-yl)-1-methyl-1H-indazol-7-yl)oxy)-N-(4-(trifluoro-methyl) thiazol-2-yl)acetamide B033. MS (ESI) m/z: 468.1 [M+1]⁺.

N-(3-Bromo-2-cyanophenyl)-2-((3-(2,6-dioxopiperidin-3-yl)-1-methyl-1H-indazol-7-yl)oxy)acetamide B034. MS (ESI) m/z: 496.1 [M+1]⁺.

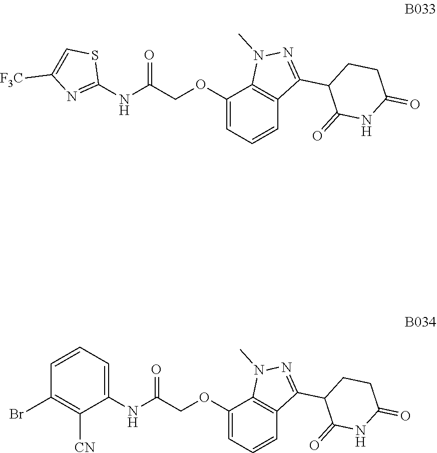 [SMILES: Cn1nc(C2CCC(=O)NC2=O)c2cccc(OCC(=O)Nc3nc(C(F)(F)F)cs3)c21 ; Cn1nc(C2CCC(=O)NC2=O)c2cccc(OCC(=O)Nc3cccc(Br)c3C#N)c21]

2-((3-(2,6-Dioxopiperidin-3-yl)-1-methyl-1H-indazol-7-yl)oxy)-N-(thiazol-4-yl-methyl)acetamide B035. MS (ESI) m/z: 414.1 [M+1]⁺.

Methyl 5-(2-((3-(2,6-dioxopiperidin-3-yl)-1-methyl-1H-indazol-7-yl)oxy)-acetamido)-3-methylthiophene-2-carboxylate B036. MS (ESI) m/z: 471.1 [M+1]⁺.

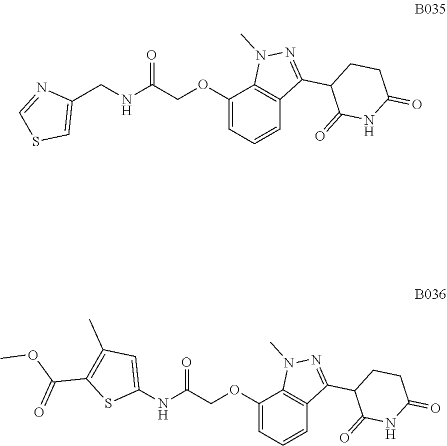 [SMILES: Cn1nc(C2CCC(=O)NC2=O)c2cccc(OCC(=O)NCc3cscn3)c21 ; COC(=O)c1sc(NC(=O)COc2cccc3c(C4CCC(=O)NC4=O)nn(C)c23)cc1C]

N-(3-Cyano-5-(trifluoromethyl)phenyl)-2-((3-(2,6-dioxopiperidin-3-yl)-1-methyl-1H-indazol-7-yl)oxy)acetamide B037. MS (ESI) m/z: 486.2 [M+1]⁺.

2-((3-(2,6-Dioxopiperidin-3-yl)-1-methyl-1H-indazol-7-yl)oxy)-N-(3-methyl-isothiazol-5-yl)acetamide B038. MS (ESI) m/z: 414.1 [M+1]⁺.

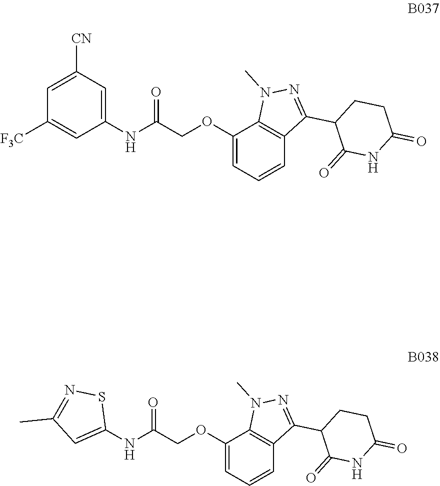 [SMILES: Cn1nc(C2CCC(=O)NC2=O)c2cccc(OCC(=O)Nc3cc(C#N)cc(C(F)(F)F)c3)c21 ; Cc1cc(NC(=O)COc2cccc3c(C4CCC(=O)NC4=O)nn(C)c23)sn1]

Methyl 2-(2-((3-(2,6-dioxopiperidin-3-yl)-1-methyl-1H-indazol-7-yl)oxy)-acetamido)-4,5-dimethylthiophene-3-carboxylate B039. MS (ESI) m/z: 485.1 [M+1]⁺.

N-(2-Cyanopyridin-3-yl)-2-((3-(2,6-dioxopiperidin-3-yl)-1-methyl-1H-indazol-7-yl)oxy)acetamide B040. MS (ESI) m/z: 419.2 [M+1]⁺.

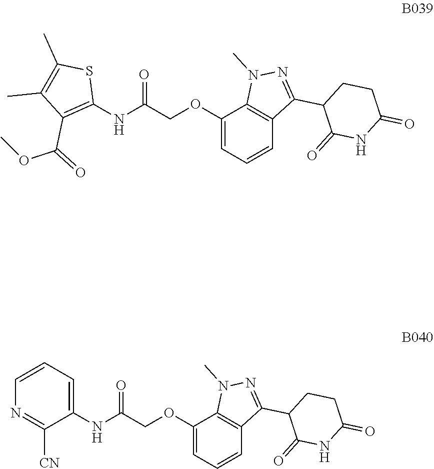 [SMILES: COC(=O)c1c(NC(=O)COc2cccc3c(C4CCC(=O)NC4=O)nn(C)c23)sc(C)c1C ; Cn1nc(C2CCC(=O)NC2=O)c2cccc(OCC(=O)Nc3cccnc3C#N)c21]

2-((3-(2,6-Dioxopiperidin-3-yl)-1-methyl-1H-indazol-7-yl)oxy)-N-(3-phenyl-1H-pyrazol-4-yl)acetamide B041. MS (ESI) m/z: 459.2 [M+1]⁺.

Methyl 2-(2-((3-(2,6-dioxopiperidin-3-yl)-1-methyl-1H-indazol-7-yl)oxy)-acetamido)-4,5,6,7-tetrahydrobenzo[b]thiophene-3-carboxylate B043. MS (ESI) m/z: 511.2 [M+1]⁺.

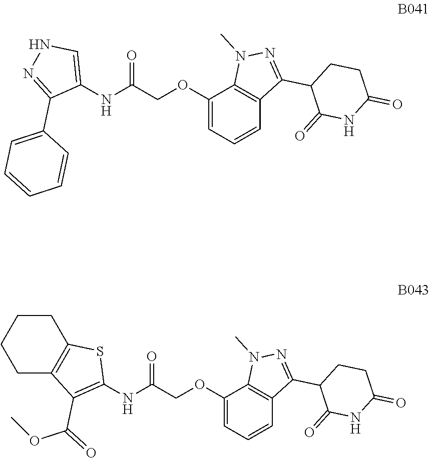 [SMILES: Cn1nc(C2CCC(=O)NC2=O)c2cccc(OCC(=O)Nc3c[nH]nc3-c3ccccc3)c21 ; COC(=O)c1c(NC(=O)COc2cccc3c(C4CCC(=O)NC4=O)nn(C)c23)sc2c1CCCC2]

5-Cyano-2-(2-((3-(2,6-dioxopiperidin-3-yl)-1-methyl-1H-indazol-7-yl)oxy)-acetamido)benzoic acid B044. MS (ESI) m/z: 462.1 [M+1]⁺.

Methyl 3-(2-((3-(2,6-dioxopiperidin-3-yl)-1-methyl-1H-indazol-7-yl)oxy)-acetamido)-5-iodothiophene-2-carboxylate B045. MS (ESI) m/z: 583 [M+1]⁺.

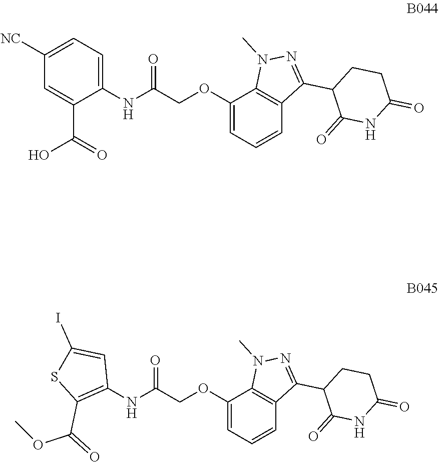 [SMILES: Cn1nc(C2CCC(=O)NC2=O)c2cccc(OCC(=O)Nc3ccc(C#N)cc3C(=O)O)c21 ; COC(=O)c1sc(I)cc1NC(=O)COc1cccc2c(C3CCC(=O)NC3=O)nn(C)c12]

N-(2-Cyano-3-fluorophenyl)-2-((3-(2,6-dioxopiperidin-3-yl)-1-methyl-1H-indazol-7-yl)oxy)acetamide B046. MS (ESI) m/z: 436.1 [M+1]⁺.

2-((3-(2,6-Dioxopiperidin-3-yl)-1-methyl-1H-indazol-7-yl)oxy)-N-(4-methyl-1,2,5-oxadiazol-3-yl)acetamide B047. MS (ESI) m/z: 399.2 [M+1]⁺.

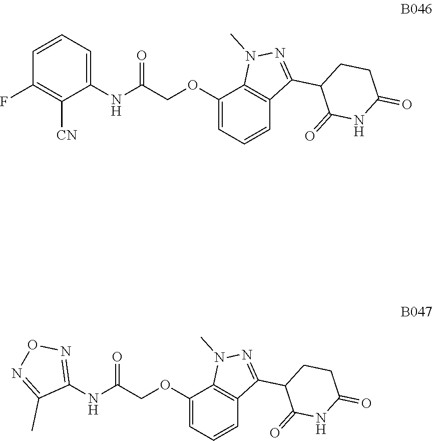 [SMILES: Cn1nc(C2CCC(=O)NC2=O)c2cccc(OCC(=O)Nc3cccc(F)c3C#N)c21 ; Cc1nonc1NC(=O)COc1cccc2c(C3CCC(=O)NC3=O)nn(C)c12]

N-(2-Cyano-4-fluorophenyl)-2-((3-(2,6-dioxopiperidin-3-yl)-1-methyl-1H-indazol-7-yl)oxy)acetamide B048. MS (ESI) m/z: 436.1 [M+1]⁺.

N-(2-Cyano-5-methylpyridin-3-yl)-2-((3-(2,6-dioxopiperidin-3-yl)-1-methyl-1H-indazol-7-yl)oxy)acetamide B049. MS (ESI) m/z: 433.2 [M+1]⁺.

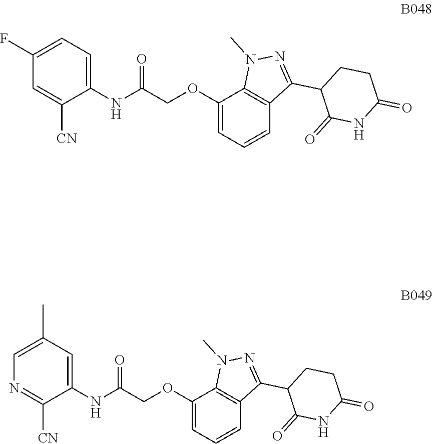 [SMILES: Cn1nc(C2CCC(=O)NC2=O)c2cccc(OCC(=O)Nc3ccc(F)cc3C#N)c21 ; Cc1cnc(C#N)c(NC(=O)COc2cccc3c(C4CCC(=O)NC4=O)nn(C)c23)c1]

N-(2-Cyanothiophen-3-yl)-2-((3-(2,6-dioxopiperidin-3-yl)-1-methyl-1H-indazol-7-yl)oxy)acetamide B050. MS (ESI) m/z: 424.1 [M+1]⁺.

N-(2-Cyano-4,6-difluorophenyl)-2-((3-(2,6-dioxopiperidin-3-yl)-1-methyl-1H-indazol-7-yl)oxy)acetamide B051. MS (ESI) m/z: 454.1 [M+1]⁺.

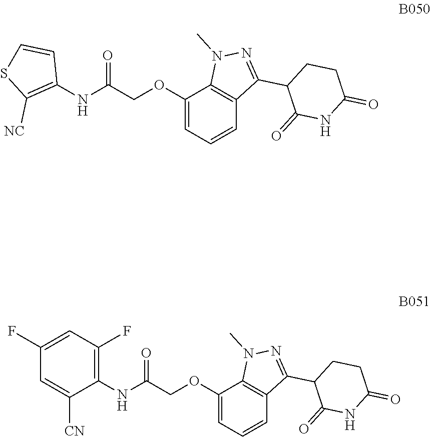 [SMILES: Cn1nc(C2CCC(=O)NC2=O)c2cccc(OCC(=O)Nc3ccsc3C#N)c21 ; Cn1nc(C2CCC(=O)NC2=O)c2cccc(OCC(=O)Nc3c(F)cc(F)cc3C#N)c21]

N-(2-Cyano-4-methylphenyl)-2-((3-(2,6-dioxopiperidin-3-yl)-1-methyl-1H-indazol-7-yl)oxy)acetamide B052. MS (ESI) m/z: 432.2 [M+1]⁺.

N-(2-Cyanobenzyl)-2-((3-(2,6-dioxopiperidin-3-yl)-1-methyl-1H-indazol-7-yl)-oxy)acetamide B053. MS (ESI) m/z: 432.2 [M+1]⁺.

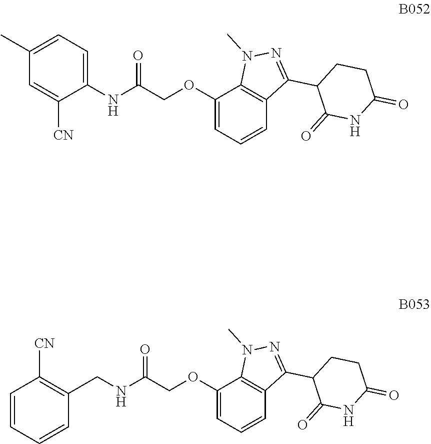 [SMILES: Cc1ccc(NC(=O)COc2cccc3c(C4CCC(=O)NC4=O)nn(C)c23)c(C#N)c1 ; Cn1nc(C2CCC(=O)NC2=O)c2cccc(OCC(=O)NCc3ccccc3C#N)c21]

N-(4-Cyano-1,3-dimethyl-1H-pyrazol-5-yl)-2-((3-(2,6-dioxopiperidin-3-yl)-1-methyl-1H-indazol-7-yl)oxy)acetamide B054. MS (ESI) m/z: 436.2 [M+1]⁺.

Methyl 3-(2-((3-(2,6-dioxopiperidin-3-yl)-1-methyl-1H-indazol-7-yl)oxy)-acetamido)-2H-thieno[3,2-c]pyrazole-5-carboxylate B055. MS (ESI) m/z: 497.1 [M+1]⁺.

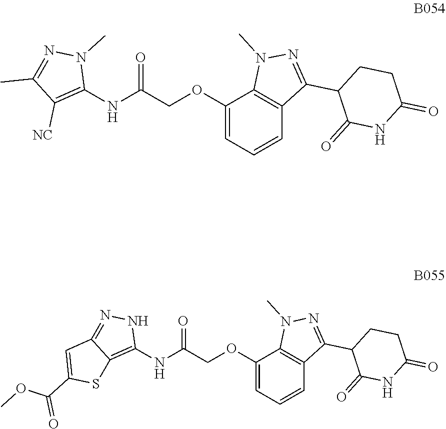 [SMILES: Cc1nn(C)c(NC(=O)COc2cccc3c(C4CCC(=O)NC4=O)nn(C)c23)c1C#N ; COC(=O)c1cc2n[nH]c(NC(=O)COc3cccc4c(C5CCC(=O)NC5=O)nn(C)c34)c2s1]

N-(2-Cyanonaphthalen-1-yl)-2-((3-(2,6-dioxopiperidin-3-yl)-1-methyl-1H-indazol-7-yl)oxy)acetamide B056. MS (ESI) m/z: 468.2 [M+1]⁺.

N-(6-Chloro-3-cyanopyridin-2-yl)-2-((3-(2,6-dioxopiperidin-3-yl)-1-methyl-1H-indazol-7-yl)oxy)acetamide B057. MS (ESI) m/z: 453.1 [M+1]⁺.

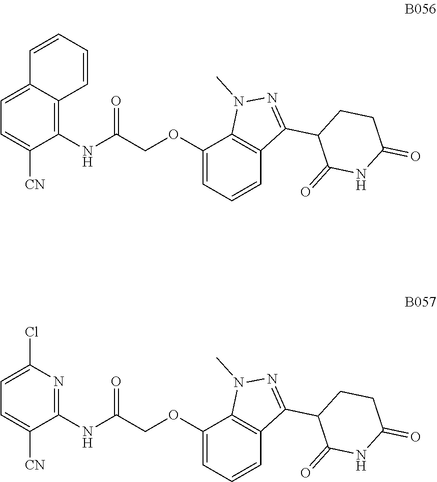 [SMILES: Cn1nc(C2CCC(=O)NC2=O)c2cccc(OCC(=O)Nc3c(C#N)ccc4ccccc34)c21 ; Cn1nc(C2CCC(=O)NC2=O)c2cccc(OCC(=O)Nc3nc(Cl)ccc3C#N)c21]

N-(3-Cyanothiophen-2-yl)-2-((3-(2,6-dioxopiperidin-3-yl)-1-methyl-1H-indazol-7-yl)oxy)acetamide B058. MS (ESI) m/z: 424.1 [M+1]⁺.

N-(2-Cyano-5-methylphenyl)-2-((3-(2,6-dioxopiperidin-3-yl)-1-methyl-1H-indazol-7-yl)oxy)acetamide B059. MS (ESI) m/z: 432.2 [M+1]⁺.

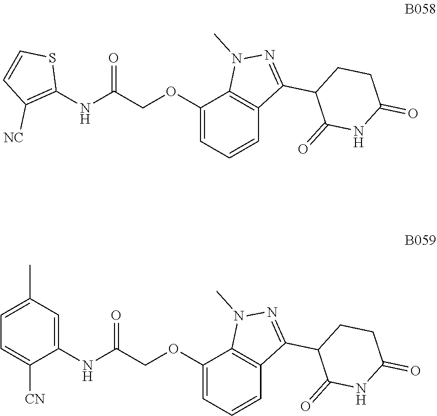 [SMILES: Cn1nc(C2CCC(=O)NC2=O)c2cccc(OCC(=O)Nc3sccc3C#N)c21 ; Cc1ccc(C#N)c(NC(=O)COc2cccc3c(C4CCC(=O)NC4=O)nn(C)c23)c1]

2-((3-(2,6-Dioxopiperidin-3-yl)-1-methyl-1H-indazol-7-yl)oxy)-N-(1-methyl-3-(trifluoromethyl)-1H-pyrazol-5-yl)acetamide B060. MS (ESI) m/z: 465.1 [M+1]⁺.

N-(4-(3-Chlorophenyl)-1H-pyrazol-5-yl)-2-((3-(2,6-dioxopiperidin-3-yl)-1-methyl-1H-indazol-7-yl)oxy)acetamide B061. MS (ESI) m/z: 493.1 [M+1]⁺.

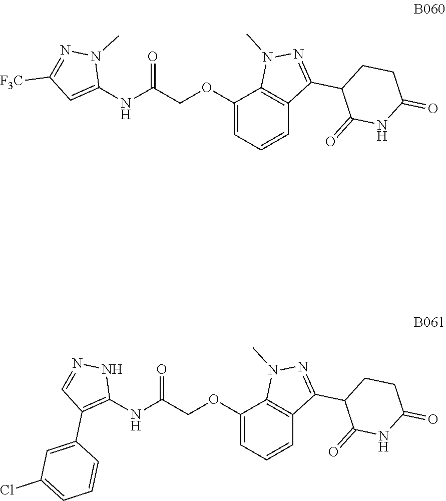 [SMILES: Cn1nc(C(F)(F)F)cc1NC(=O)COc1cccc2c(C3CCC(=O)NC3=O)nn(C)c12 ; Cn1nc(C2CCC(=O)NC2=O)c2cccc(OCC(=O)Nc3[nH]ncc3-c3cccc(Cl)c3)c21]

N-(2-Cyano-6-fluorophenyl)-2-((3-(2,6-dioxopiperidin-3-yl)-1-methyl-1H-indazol-7-yl)oxy)acetamide B062. MS (ESI) m/z: 436.2 [M+1]⁺.

Ethyl 2-(2-((3-(2,6-dioxopiperidin-3-yl)-1-methyl-1H-indazol-7-yl)oxy)-acetamido)-4,5,6,7-tetrahydrobenzo[b]thiophene-3-carboxylate B063. MS (ESI) m/z: 525.2 [M+1]⁺.

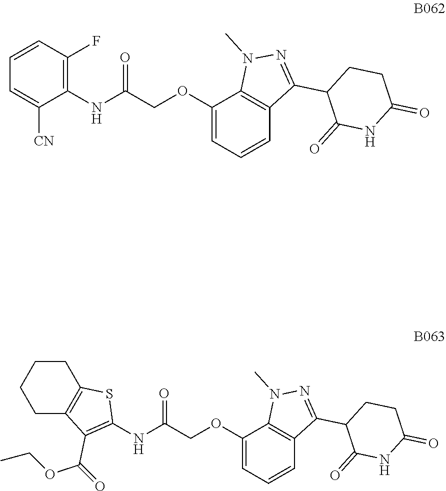 [SMILES: Cn1nc(C2CCC(=O)NC2=O)c2cccc(OCC(=O)Nc3c(F)cccc3C#N)c21 ; CCOC(=O)c1c(NC(=O)COc2cccc3c(C4CCC(=O)NC4=O)nn(C)c23)sc2c1CCCC2]

N-((1-Cyanocyclopentyl)methyl)-2-((3-(2,6-dioxopiperidin-3-yl)-1-methyl-1H-indazol-7-yl)oxy)acetamide B064. MS (ESI) m/z: 424.2 [M+1]⁺.

N-(4-Cyano-1-methyl-1H-pyrazol-5-yl)-2-((3-(2,6-dioxopiperidin-3-yl)-1-methyl-1H-indazol-7-yl)oxy)acetamide B065. MS (ESI) m/z: 422.2 [M+1]⁺.

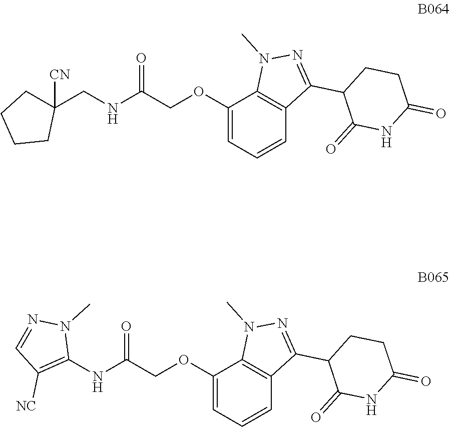 [SMILES: Cn1nc(C2CCC(=O)NC2=O)c2cccc(OCC(=O)NCC3(C#N)CCCC3)c21 ; Cn1ncc(C#N)c1NC(=O)COc1cccc2c(C3CCC(=O)NC3=O)nn(C)c12]

N-(4-Cyano-3-methylisoxazol-5-yl)-2-((3-(2,6-dioxopiperidin-3-yl)-1-methyl-1H-indazol-7-yl)oxy)acetamide B066. MS (ESI) m/z: 423.2 [M+1]⁺.

N-(1-(Tert-butyl)-3-methyl-1H-pyrazol-5-yl)-2-((3-(2,6-dioxopiperidin-3-yl)-1-methyl-1H-indazol-7-yl)oxy)acetamide B067. MS (ESI) m/z: 453.2 [M+1]⁺.

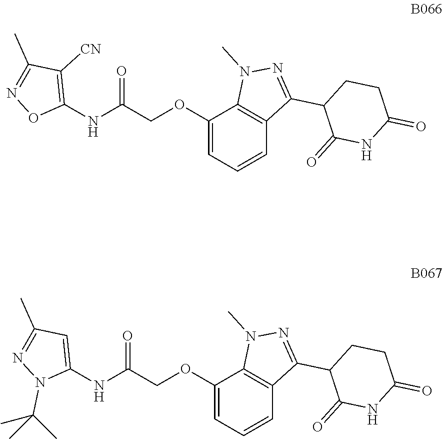 [SMILES: Cc1noc(NC(=O)COc2cccc3c(C4CCC(=O)NC4=O)nn(C)c23)c1C#N ; Cc1cc(NC(=O)COc2cccc3c(C4CCC(=O)NC4=O)nn(C)c23)n(C(C)(C)C)n1]

2-((3-(2,6-Dioxopiperidin-3-yl)-1-methyl-1H-indazol-7-yl)oxy)-N-(5-(furan-2-yl)-1,3,4-oxadiazol-2-yl)acetamide B068. MS (ESI) m/z: 451.1 [M+1]⁺.

2-((3-(2,6-Dioxopiperidin-3-yl)-1-methyl-1H-indazol-7-yl)oxy)-N-(5-methyl-1,3,4-oxadiazol-2-yl)acetamide B069. MS (ESI) m/z: 399.1 [M+1]⁺.

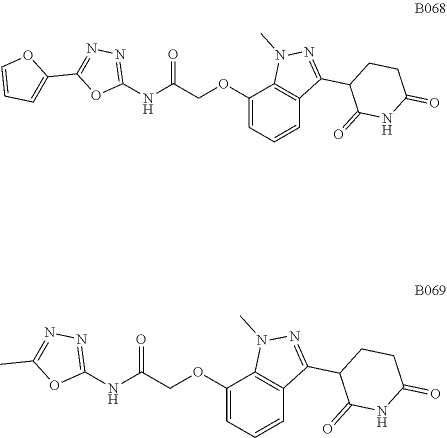 [SMILES: Cn1nc(C2CCC(=O)NC2=O)c2cccc(OCC(=O)Nc3nnc(-c4ccco4)o3)c21 ; Cc1nnc(NC(=O)COc2cccc3c(C4CCC(=O)NC4=O)nn(C)c23)o1]

N-(3,5-Dimethylisoxazol-4-yl)-2-((3-(2,6-dioxopiperidin-3-yl)-1-methyl-1H-indazol-7-yl)oxy)acetamide B070. MS (ESI) m/z: 412.2 [M+1]⁺.

2-((3-(2,6-Dioxopiperidin-3-yl)-1-methyl-1H-indazol-7-yl)oxy)-N-((4-hydroxy-tetrahydro-2H-pyran-4-yl)methyl)acetamide B071. MS (ESI) m/z: 431.2 [M+1]⁺.

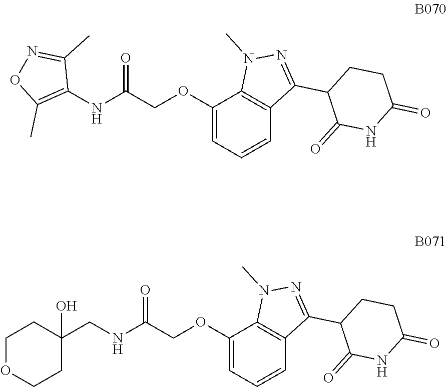 [SMILES: Cc1noc(C)c1NC(=O)COc1cccc2c(C3CCC(=O)NC3=O)nn(C)c12 ; Cn1nc(C2CCC(=O)NC2=O)c2cccc(OCC(=O)NCC3(O)CCOCC3)c21]

2-((3-(2,6-Dioxopiperidin-3-yl)-1-methyl-1H-indazol-7-yl)oxy)-N-(5-ethyl-isoxazol-3-yl)acetamide B072. MS (ESI) m/z: 412.2 [M+1]⁺.

2-((3-(2,6-Dioxopiperidin-3-yl)-1-methyl-1H-indazol-7-yl)oxy)-N-(3-ethyl-isoxazol-5-yl)acetamide B073. MS (ESI) m/z: 412 [M+1]⁺.

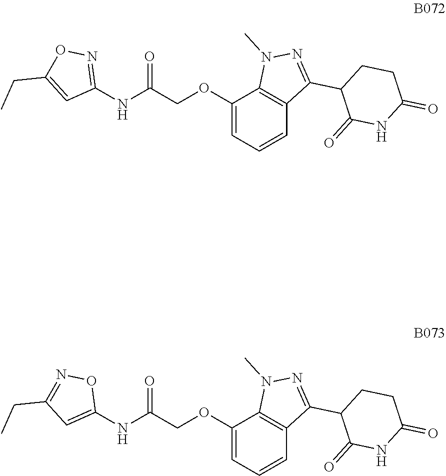 [SMILES: CCc1cc(NC(=O)COc2cccc3c(C4CCC(=O)NC4=O)nn(C)c23)no1 ; CCc1cc(NC(=O)COc2cccc3c(C4CCC(=O)NC4=O)nn(C)c23)on1]

2-((3-(2,6-Dioxopiperidin-3-yl)-1-methyl-1H-indazol-7-yl)oxy)-N-(3-methyl-isoxazol-5-yl)acetamide B074. MS (ESI) m/z: 398.2 [M+1]⁺.

2-((3-(2,6-Dioxopiperidin-3-yl)-1-methyl-1H-indazol-7-yl)oxy)-N-(isoxazol-5-yl)acetamide B075. MS (ESI) m/z: 384.2 [M+1]⁺.

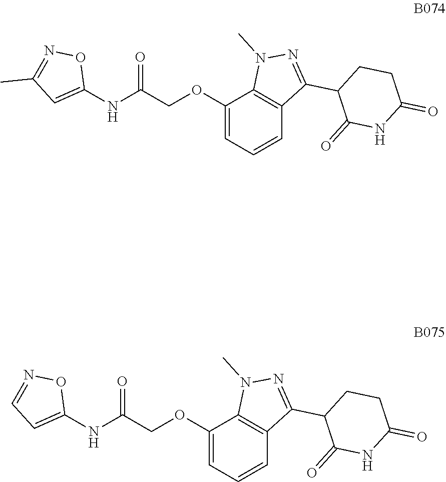 [SMILES: Cc1cc(NC(=O)COc2cccc3c(C4CCC(=O)NC4=O)nn(C)c23)on1 ; Cn1nc(C2CCC(=O)NC2=O)c2cccc(OCC(=O)Nc3ccno3)c21]

2-((3-(2,6-Dioxopiperidin-3-yl)-1-methyl-1H-indazol-7-yl)oxy)-N-(1-methyl-1H-1,2,4-triazol-5-yl)acetamide B076. MS (ESI) m/z: 398.2 [M+1]⁺.

2-((3-(2,6-Dioxopiperidin-3-yl)-1-methyl-1H-indazol-7-yl)oxy)-N-((8-methyl-[1,2,4]triazolo[4,3-a]pyridin-3-yl)methyl)acetamide B077. MS (ESI) m/z: 462.2 [M+1]⁺.

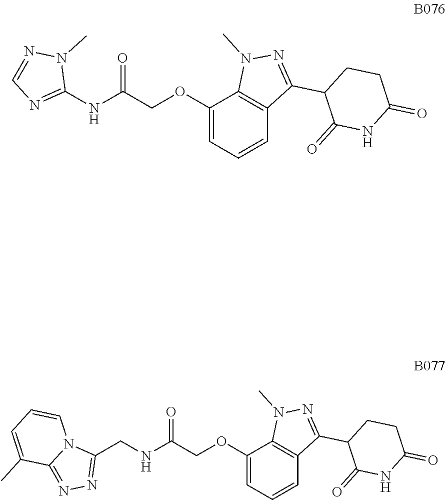 [SMILES: Cn1ncnc1NC(=O)COc1cccc2c(C3CCC(=O)NC3=O)nn(C)c12 ; Cc1cccn2c(CNC(=O)COc3cccc4c(C5CCC(=O)NC5=O)nn(C)c34)nnc12]

2-((3-(2,6-Dioxopiperidin-3-yl)-1-methyl-1H-indazol-7-yl)oxy)-N-(4-(2-methyl-1H-imidazol-1-yl)phenyl)acetamide B078. MS (ESI) m/z: 473.2 [M+1]⁺.

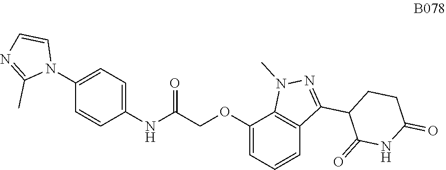 [SMILES: Cc1nccn1-c1ccc(NC(=O)COc2cccc3c(C4CCC(=O)NC4=O)nn(C)c23)cc1]

2-((3-(2,6-Dioxopiperidin-3-yl)-1-methyl-1H-indazol-7-yl)oxy)-N-((6-ethoxy-pyridin-3-yl)methyl)acetamide B079. MS (ESI) m/z: 452.2 [M+1]⁺.

2-((3-(2,6-Dioxopiperidin-3-yl)-1-methyl-1H-indazol-7-yl)oxy)-N—(((R)-5-oxo-pyrrolidin-2-yl)methyl)acetamide B080. MS (ESI) m/z: 414.2 [M+1]⁺.

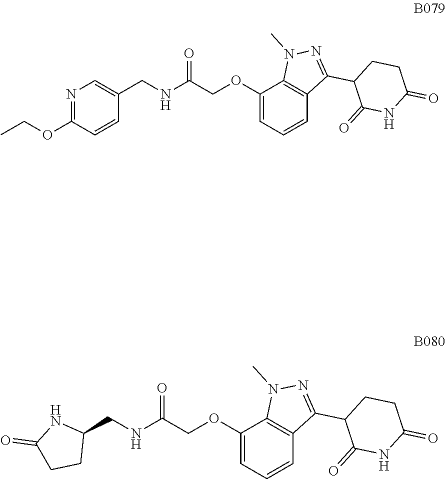 [SMILES: CCOc1ccc(CNC(=O)COc2cccc3c(C4CCC(=O)NC4=O)nn(C)c23)cn1 ; Cn1nc(C2CCC(=O)NC2=O)c2cccc(OCC(=O)NC[C@H]3CCC(=O)N3)c21]

2-((3-(2,6-Dioxopiperidin-3-yl)-1-methyl-1H-indazol-7-yl)oxy)-N-(1-methyl-2-oxopiperidin-4-yl)acetamide B081. MS (ESI) m/z: 428.2 [M+1]⁺.

2-((3-(2,6-Dioxopiperidin-3-yl)-1-methyl-1H-indazol-7-yl)oxy)-N-(2-(5-isopropyl-1,2,4-oxadiazol-3-yl)ethyl)acetamide B082. MS (ESI) m/z: 455.2 [M+1]⁺.

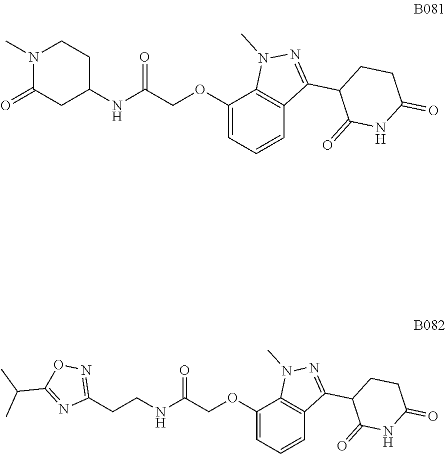 [SMILES: CN1CCC(NC(=O)COc2cccc3c(C4CCC(=O)NC4=O)nn(C)c23)CC1=O ; CC(C)c1nc(CCNC(=O)COc2cccc3c(C4CCC(=O)NC4=O)nn(C)c23)no1]

2-((3-(2,6-Dioxopiperidin-3-yl)-1-methyl-1H-indazol-7-yl)oxy)-N-(4-(1-methyl-piperidin-4-yl)phenyl)acetamide B083. MS (ESI) m/z: 490.2 [M+1]⁺.

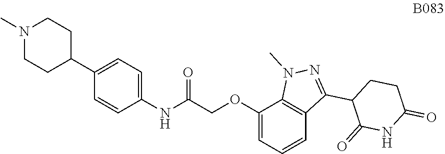 [SMILES: CN1CCC(c2ccc(NC(=O)COc3cccc4c(C5CCC(=O)NC5=O)nn(C)c34)cc2)CC1]

2-((3-(2,6-Dioxopiperidin-3-yl)-1-methyl-1H-indazol-7-yl)oxy)-N-(4-(N-methyl-sulfamoyl)phenyl)acetamide B084. MS (ESI) m/z: 486.1 [M+1]⁺.

2-((3-(2,6-Dioxopiperidin-3-yl)-1-methyl-1H-indazol-7-yl)oxy)-N—((R)-2-oxo-azepan-3-yl)acetamide B085. MS (ESI) m/z: 428.2 [M+1]⁺.

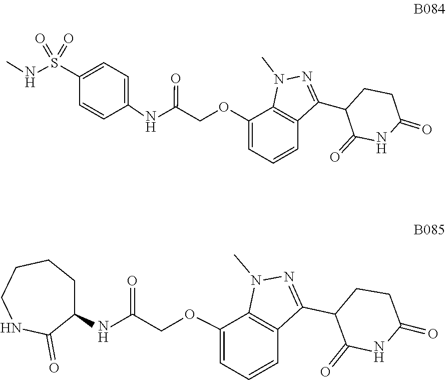 [SMILES: CNS(=O)(=O)c1ccc(NC(=O)COc2cccc3c(C4CCC(=O)NC4=O)nn(C)c23)cc1 ; Cn1nc(C2CCC(=O)NC2=O)c2cccc(OCC(=O)N[C@@H]3CCCCNC3=O)c21]

N-(4-(1H-Imidazol-2-yl)phenyl)-2-((3-(2,6-dioxopiperidin-3-yl)-1-methyl-1H-indazol-7-yl)oxy)acetamide B086. MS (ESI) m/z: 459.1 [M+1]⁺.

2-((3-(2,6-Dioxopiperidin-3-yl)-1-methyl-1H-indazol-7-yl)oxy)-N-(thiazol-4-yl)-acetamide B087. MS (ESI) m/z: 400.1 [M+1]⁺.

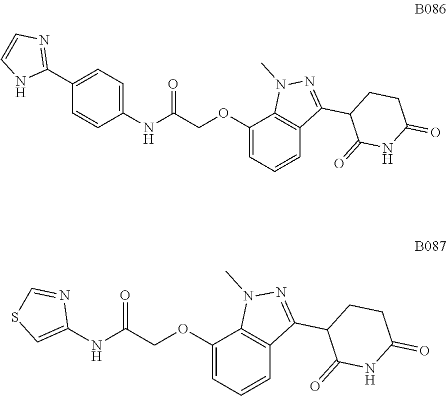 [SMILES: Cn1nc(C2CCC(=O)NC2=O)c2cccc(OCC(=O)Nc3ccc(-c4ncc[nH]4)cc3)c21 ; Cn1nc(C2CCC(=O)NC2=O)c2cccc(OCC(=O)Nc3cscn3)c21]

2-((3-(2,6-Dioxopiperidin-3-yl)-1-methyl-1H-indazol-7-yl)oxy)-N-(4-(methyl-sulfonyl)phenyl)acetamide B088. MS (ESI) m/z: 471.2 [M+1]⁺.

2-((3-(2,6-Dioxopiperidin-3-yl)-1-methyl-1H-indazol-7-yl)oxy)-N-(pyridazin-3-yl)acetamide B089. MS (ESI) m/z: 395.2 [M+1]⁺.

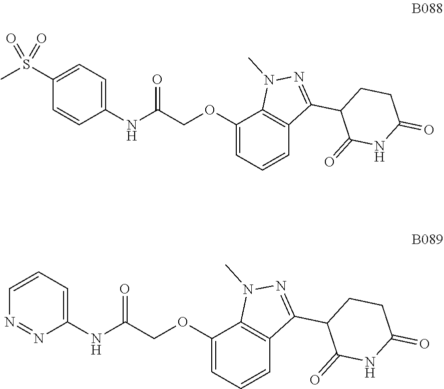 [SMILES: Cn1nc(C2CCC(=O)NC2=O)c2cccc(OCC(=O)Nc3ccc(S(C)(=O)=O)cc3)c21 ; Cn1nc(C2CCC(=O)NC2=O)c2cccc(OCC(=O)Nc3cccnn3)c21]

2-((3-(2,6-Dioxopiperidin-3-yl)-1-methyl-1H-indazol-7-yl)oxy)-N-(4-(4-methyl-piperazin-1-yl)phenyl)acetamide B090. MS (ESI) m/z: 491.3 [M+1]⁺.

2-((3-(2,6-Dioxopiperidin-3-yl)-1-methyl-1H-indazol-7-yl)oxy)-N—((R)-1-(pyridin-2-yl)ethyl)acetamide B091. MS (ESI) m/z: 422.2 [M+1]⁺.

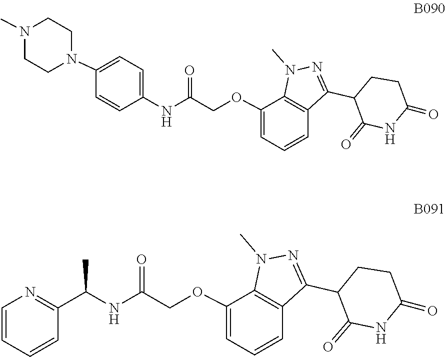 [SMILES: CN1CCN(c2ccc(NC(=O)COc3cccc4c(C5CCC(=O)NC5=O)nn(C)c34)cc2)CC1 ; C[C@@H](NC(=O)COc1cccc2c(C3CCC(=O)NC3=O)nn(C)c12)c1ccccn1]

2-((3-(2,6-Dioxopiperidin-3-yl)-1-methyl-1H-indazol-7-yl)oxy)-N-(oxazol-2-yl)-acetamide B092. MS (ESI) m/z: 384.2 [M+1]⁺.

2-((3-(2,6-Dioxopiperidin-3-yl)-1-methyl-1H-indazol-7-yl)oxy)-N-(4-(2-oxo-pyrrolidin-1-yl)benzyl)acetamide B093. MS (ESI) m/z: 490.2 [M+1]⁺.

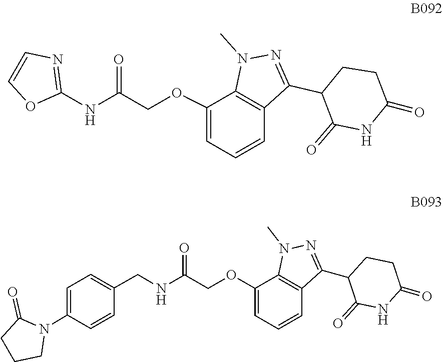 [SMILES: Cn1nc(C2CCC(=O)NC2=O)c2cccc(OCC(=O)Nc3ncco3)c21 ; Cn1nc(C2CCC(=O)NC2=O)c2cccc(OCC(=O)NCc3ccc(N4CCCC4=O)cc3)c21]

2-((3-(2,6-Dioxopiperidin-3-yl)-1-methyl-1H-indazol-7-yl)oxy)-N-(oxetan-3-yl)-acetamide B094. MS (ESI) m/z: 373.1 [M+1]⁺.

2-((3-(2,6-Dioxopiperidin-3-yl)-1-methyl-1H-indazol-7-yl)oxy)-N-(1-methyl-1H-pyrazol-4-yl)acetamide B095. MS (ESI) m/z: 397.1 [M+1]⁺.

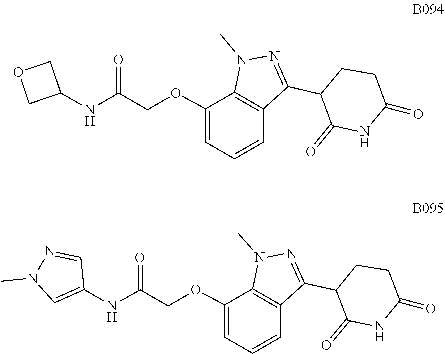 [SMILES: Cn1nc(C2CCC(=O)NC2=O)c2cccc(OCC(=O)NC3COC3)c21 ; Cn1cc(NC(=O)COc2cccc3c(C4CCC(=O)NC4=O)nn(C)c23)cn1]

2-((3-(2,6-Dioxopiperidin-3-yl)-1-methyl-1H-indazol-7-yl)oxy)-N—(((S)-oxetan-2-yl)methyl)acetamide B096. MS (ESI) m/z: 387.1 [M+1]⁺.

N-((2-Chlorothiazol-5-yl)methyl)-2-((3-(2,6-dioxopiperidin-3-yl)-1-methyl-1H-indazol-7-yl)oxy)acetamide B097. MS (ESI) m/z: 448.1 [M+1]⁺.

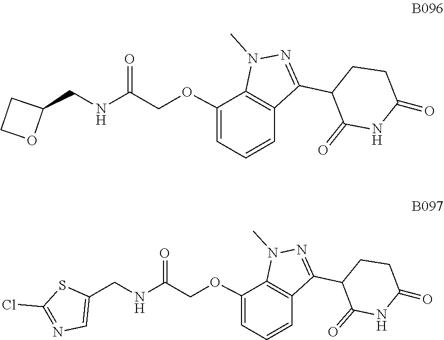 [SMILES: Cn1nc(C2CCC(=O)NC2=O)c2cccc(OCC(=O)NC[C@@H]3CCO3)c21 ; Cn1nc(C2CCC(=O)NC2=O)c2cccc(OCC(=O)NCc3cnc(Cl)s3)c21]

2-((3-(2,6-Dioxopiperidin-3-yl)-1-methyl-1H-indazol-7-yl)oxy)-N—((R)-1-(4-fluorophenyl)ethyl)acetamide B098. MS (ESI) m/z: 439.2 [M+1]⁺.

2-((3-(2,6-Dioxopiperidin-3-yl)-1-methyl-1H-indazol-7-yl)oxy)-N—((S)-1-phenyl-propyl)acetamide B099. MS (ESI) m/z: 435.2 [M+1]⁺.

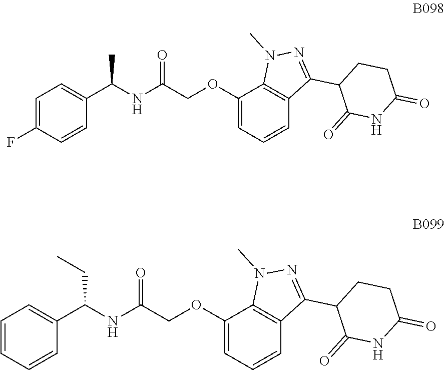 [SMILES: C[C@@H](NC(=O)COc1cccc2c(C3CCC(=O)NC3=O)nn(C)c12)c1ccc(F)cc1 ; CC[C@H](NC(=O)COc1cccc2c(C3CCC(=O)NC3=O)nn(C)c12)c1ccccc1]

N—((S)-1-(4-Chlorophenyl)ethyl)-2-((3-(2,6-dioxopiperidin-3-yl)-1-methyl-1H-indazol-7-yl)oxy)acetamide B100. MS (ESI) m/z: 455.1 [M+1]⁺.

2-((3-(2,6-Dioxopiperidin-3-yl)-1-methyl-1H-indazol-7-yl)oxy)-N-(1-phenyl-1H-pyrazol-4-yl)acetamide B101. MS (ESI) m/z: 459.1 [M+1]⁺.

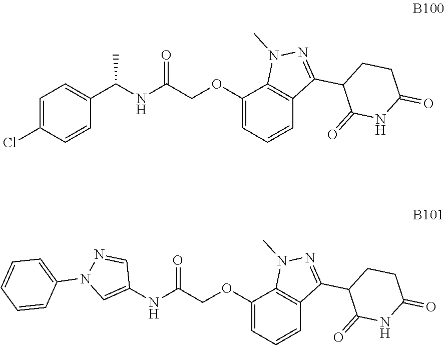 [SMILES: C[C@H](NC(=O)COc1cccc2c(C3CCC(=O)NC3=O)nn(C)c12)c1ccc(Cl)cc1 ; Cn1nc(C2CCC(=O)NC2=O)c2cccc(OCC(=O)Nc3cnn(-c4ccccc4)c3)c21]

2-((3-(2,6-Dioxopiperidin-3-yl)-1-methyl-1H-indazol-7-yl)oxy)-N—((S)-1-(p-tolyl)ethyl)acetamide B102. MS (ESI) m/z: 435.2 [M+1]⁺.

N-(3,5-Dimethyl-1H-pyrazol-4-yl)-2-((3-(2,6-dioxopiperidin-3-yl)-1-methyl-1H-indazol-7-yl)oxy)acetamide B103. MS (ESI) m/z: 411.2 [M+1]⁺.

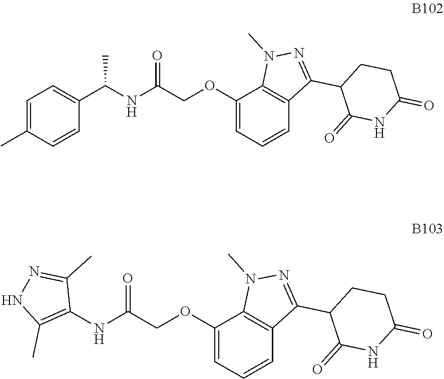 [SMILES: Cc1ccc([C@H](C)NC(=O)COc2cccc3c(C4CCC(=O)NC4=O)nn(C)c23)cc1 ; Cc1n[nH]c(C)c1NC(=O)COc1cccc2c(C3CCC(=O)NC3=O)nn(C)c12]

N-(3-(Difluoromethoxy)-1H-pyrazol-5-yl)-2-((3-(2,6-dioxopiperidin-3-yl)-1-methyl-1H-indazol-7-yl)oxy)acetamide B104. MS (ESI) m/z: 449.1 [M+1]⁺.

2-((3-(2,6-Dioxopiperidin-3-yl)-1-methyl-1H-indazol-7-yl)oxy)-N-(2-(methyl-amino)-2-oxoethyl)acetamide B105. MS (ESI) m/z: 388.2 [M+1]⁺.

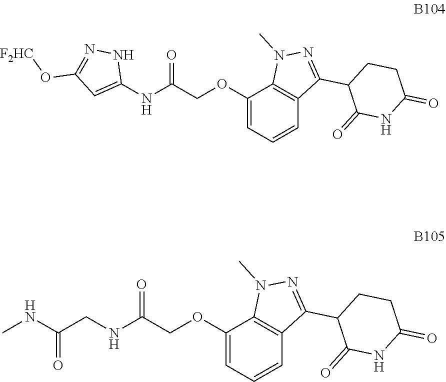 [SMILES: Cn1nc(C2CCC(=O)NC2=O)c2cccc(OCC(=O)Nc3cc(OC(F)F)n[nH]3)c21 ; CNC(=O)CNC(=O)COc1cccc2c(C3CCC(=O)NC3=O)nn(C)c12]

2-((3-(2,6-Dioxopiperidin-3-yl)-1-methyl-1H-indazol-7-yl)oxy)-N-((1s,4s)-4-hydroxycyclohexyl)acetamide B106. MS (ESI) m/z: 415.2 [M+1]⁺.

2-((3-(2,6-Dioxopiperidin-3-yl)-1-methyl-1H-indazol-7-yl)oxy)-N-(2-oxo-piperidin-3-yl)acetamide B107. MS (ESI) m/z: 414.1 [M+1]⁺.

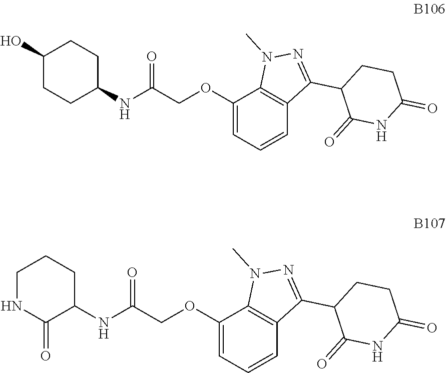 [SMILES: Cn1nc(C2CCC(=O)NC2=O)c2cccc(OCC(=O)N[C@H]3CC[C@@H](O)CC3)c21 ; Cn1nc(C2CCC(=O)NC2=O)c2cccc(OCC(=O)NC3CCCNC3=O)c21]

2-((3-(2,6-Dioxopiperidin-3-yl)-1-methyl-1H-indazol-7-yl)oxy)-N-((1r,4r)-4-methoxycyclohexyl)acetamide B108. MS (ESI) m/z: 429.2 [M+1]⁺.

N-(3-(Tert-butyl)-1-phenyl-1H-pyrazol-5-yl)-2-((3-(2,6-dioxopiperidin-3-yl)-1-methyl-1H-indazol-7-yl)oxy)acetamide B109. MS (ESI) m/z: 515.3 [M+1]⁺.

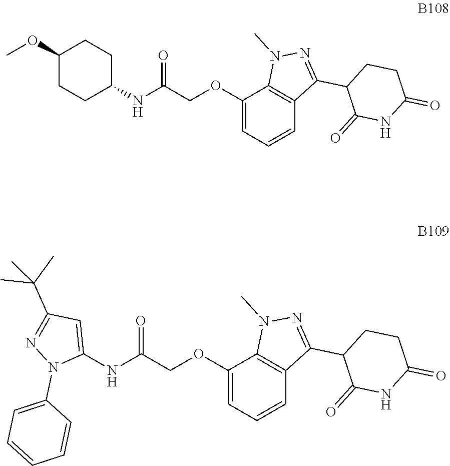 [SMILES: CO[C@H]1CC[C@H](NC(=O)COc2cccc3c(C4CCC(=O)NC4=O)nn(C)c23)CC1 ; Cn1nc(C2CCC(=O)NC2=O)c2cccc(OCC(=O)Nc3cc(C(C)(C)C)nn3-c3ccccc3)c21]

N-(4-(1H-Pyrrol-1-yl)phenyl)-2-((3-(2,6-dioxopiperidin-3-yl)-1-methyl-1H-indazol-7-yl)oxy)acetamide B110. MS (ESI) m/z: 458.2 [M+1]⁺.

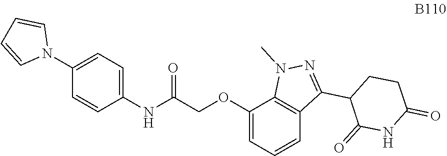 [SMILES: Cn1nc(C2CCC(=O)NC2=O)c2cccc(OCC(=O)Nc3ccc(-n4cccc4)cc3)c21]

2-((3-(2,6-Dioxopiperidin-3-yl)-1-methyl-1H-indazol-7-yl)oxy)-N-(4-hydroxy-phenethyl)acetamide B111. MS (ESI) m/z: 437.1 [M+1]⁺.

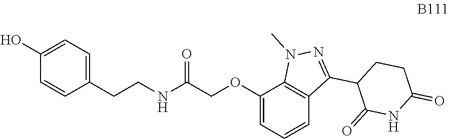 [SMILES: Cn1nc(C2CCC(=O)NC2=O)c2cccc(OCC(=O)NCCc3ccc(O)cc3)c21]

2-((3-(2,6-Dioxopiperidin-3-yl)-1-methyl-1H-indazol-7-yl)oxy)-N-((2-methyl-1H-indol-5-yl)methyl)acetamide B112. MS (ESI) m/z: 460.2 [M+1]⁺.

2-((3-(2,6-Dioxopiperidin-3-yl)-1-methyl-1H-indazol-7-yl)oxy)-N-(2-methyl-thiazol-5-yl)acetamide B113. MS (ESI) m/z: 414.1 [M+1]⁺.

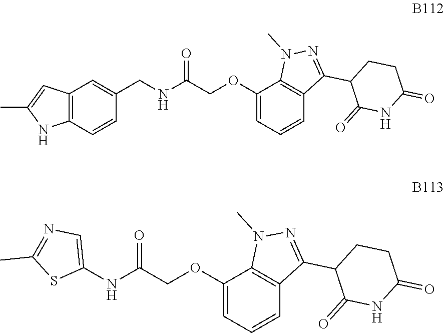 [SMILES: Cc1cc2cc(CNC(=O)COc3cccc4c(C5CCC(=O)NC5=O)nn(C)c34)ccc2[nH]1 ; Cc1ncc(NC(=O)COc2cccc3c(C4CCC(=O)NC4=O)nn(C)c23)s1]

2-((3-(2,6-Dioxopiperidin-3-yl)-1-methyl-1H-indazol-7-yl)oxy)-N-(pyridin-2-yl)-acetamide B114. MS (ESI) m/z: 394.1 [M+1]⁺.

N-(4-(Tert-butyl) thiazol-2-yl)-2-((3-(2,6-dioxopiperidin-3-yl)-1-methyl-1H-indazol-7-yl)oxy)acetamide B115. MS (ESI) m/z: 456.2 [M+1]⁺.

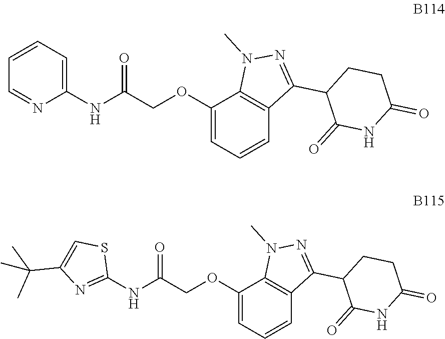 [SMILES: Cn1nc(C2CCC(=O)NC2=O)c2cccc(OCC(=O)Nc3ccccn3)c21 ; Cn1nc(C2CCC(=O)NC2=O)c2cccc(OCC(=O)Nc3nc(C(C)(C)C)cs3)c21]

2-((3-(2,6-Dioxopiperidin-3-yl)-1-methyl-1H-indazol-7-yl)oxy)-N-((3-hydroxy-cyclopentyl)methyl)acetamide B116. MS (ESI) m/z: 415.2 [M+1]⁺.

2-((3-(2,6-Dioxopiperidin-3-yl)-1-methyl-1H-indazol-7-yl)oxy)-N-(3-methyl-isothiazol-4-yl)acetamide B117. MS (ESI) m/z: 414.1 [M+1]⁺.

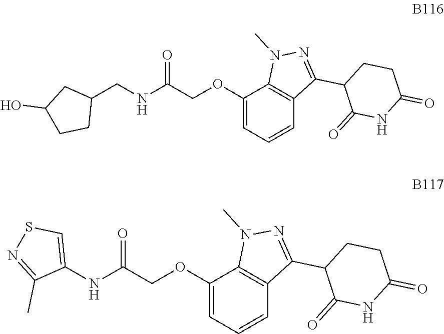 [SMILES: Cn1nc(C2CCC(=O)NC2=O)c2cccc(OCC(=O)NCC3CCC(O)C3)c21 ; Cc1nscc1NC(=O)COc1cccc2c(C3CCC(=O)NC3=O)nn(C)c12]

2-((3-(2,6-Dioxopiperidin-3-yl)-1-methyl-1H-indazol-7-yl)oxy)-N-(4-((pyrrolidin-1-ylsulfonyl)methyl)phenyl)acetamide B118. MS (ESI) m/z: 540.2 [M+1]⁺.

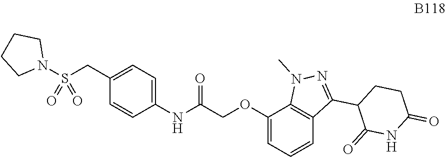 [SMILES: Cn1nc(C2CCC(=O)NC2=O)c2cccc(OCC(=O)Nc3ccc(CS(=O)(=O)N4CCCC4)cc3)c21]

N-(4-(Sec-butoxy)benzyl)-2-((3-(2,6-dioxopiperidin-3-yl)-1-methyl-1H-indazol-7-yl)oxy)acetamide B119. MS (ESI) m/z: 479.2 [M+1]⁺.

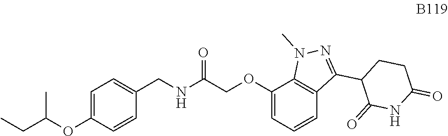 [SMILES: CCC(C)Oc1ccc(CNC(=O)COc2cccc3c(C4CCC(=O)NC4=O)nn(C)c23)cc1]

2-((3-(2,6-Dioxopiperidin-3-yl)-1-methyl-1H-indazol-7-yl)oxy)-N-(1-methyl-piperidin-4-yl)acetamide B120. MS (ESI) m/z: 414.1 [M+1]⁺.

2-((3-(2,6-Dioxopiperidin-3-yl)-1-methyl-1H-indazol-7-yl)oxy)-N-(3-hydroxy-cyclopentyl)acetamide B121. MS (ESI) m/z: 401.1 [M+1]⁺.

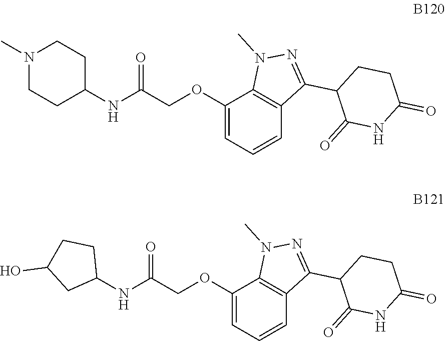 [SMILES: CN1CCC(NC(=O)COc2cccc3c(C4CCC(=O)NC4=O)nn(C)c23)CC1 ; Cn1nc(C2CCC(=O)NC2=O)c2cccc(OCC(=O)NC3CCC(O)C3)c21]

2-((3-(2,6-Dioxopiperidin-3-yl)-1-methyl-1H-indazol-7-yl)oxy)-N-(3-fluoro-5-morpholinophenyl)acetamide B122. MS (ESI) m/z: 496.1 [M+1]⁺.

N-(2-(3,5-Dimethyl-1H-1,2,4-triazol-1-yl)ethyl)-2-((3-(2,6-dioxopiperidin-3-yl)-1-methyl-1H-indazol-7-yl)oxy)acetamide B123. MS (ESI) m/z: 440.1 [M+1]⁺.

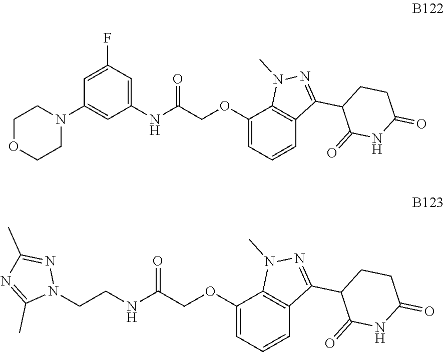 [SMILES: Cn1nc(C2CCC(=O)NC2=O)c2cccc(OCC(=O)Nc3cc(F)cc(N4CCOCC4)c3)c21 ; Cc1nc(C)n(CCNC(=O)COc2cccc3c(C4CCC(=O)NC4=O)nn(C)c23)n1]

N-(4-Cyano-1-(2-hydroxyethyl)-1H-pyrazol-5-yl)-2-((3-(2,6-dioxopiperidin-3-yl)-1-methyl-1H-indazol-7-yl)oxy)acetamide B124. MS (ESI) m/z: 452.1 [M+1]⁺.

2-((3-(2,6-Dioxopiperidin-3-yl)-1-methyl-1H-indazol-7-yl)oxy)-N-((1-methyl-1H-imidazol-5-yl)methyl)acetamide B125. MS (ESI) m/z: 411.1 [M+1]⁺.

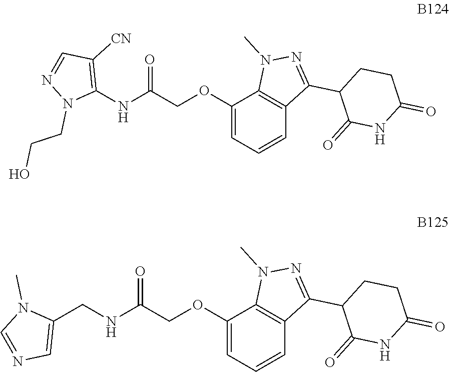 [SMILES: Cn1nc(C2CCC(=O)NC2=O)c2cccc(OCC(=O)Nc3c(C#N)cnn3CCO)c21 ; Cn1cncc1CNC(=O)COc1cccc2c(C3CCC(=O)NC3=O)nn(C)c12]

N-(1,1-Dioxidotetrahydro-2H-thiopyran-4-yl)-2-((3-(2,6-dioxopiperidin-3-yl)-1-methyl-1H-indazol-7-yl)oxy)acetamide B126. MS (ESI) m/z: 449.1 [M+1]⁺.

N-(4-Cyanobicyclo[2.2.2]octan-1-yl)-2-((3-(2,6-dioxopiperidin-3-yl)-1-methyl-1H-indazol-7-yl)oxy)acetamide B127. MS (ESI) m/z: 450.1 [M+1]⁺.

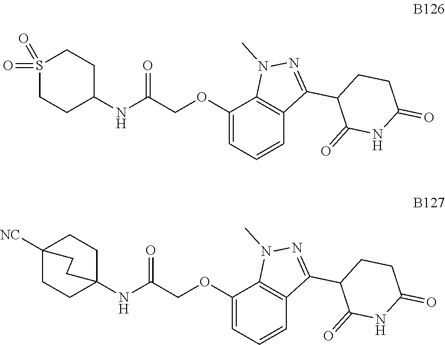 [SMILES: Cn1nc(C2CCC(=O)NC2=O)c2cccc(OCC(=O)NC3CCS(=O)(=O)CC3)c21 ; Cn1nc(C2CCC(=O)NC2=O)c2cccc(OCC(=O)NC34CCC(C#N)(CC3)CC4)c21]

N-(3-Cyanobenzyl)-2-((3-(2,6-dioxopiperidin-3-yl)-1-methyl-1H-indazol-7-yl)-oxy)acetamide B128. MS (ESI) m/z: 432.1 [M+1]⁺.

2-((3-(2,6-Dioxopiperidin-3-yl)-1-methyl-1H-indazol-7-yl)oxy)-N-((1-methyl-2-oxo-1,2-dihydropyridin-4-yl)methyl)acetamide B129. MS (ESI) m/z: 438.1 [M+1]⁺.

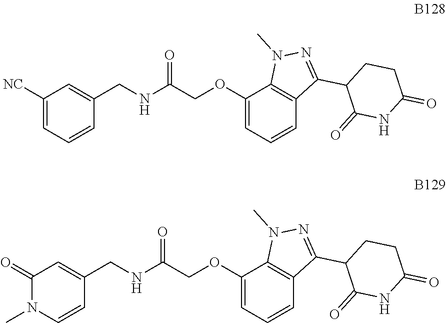 [SMILES: Cn1nc(C2CCC(=O)NC2=O)c2cccc(OCC(=O)NCc3cccc(C#N)c3)c21 ; Cn1ccc(CNC(=O)COc2cccc3c(C4CCC(=O)NC4=O)nn(C)c23)cc1=O]

N-(3-Cyclopropyl-1-methyl-1H-pyrazol-5-yl)-2-((3-(2,6-dioxopiperidin-3-yl)-1-methyl-1H-indazol-7-yl)oxy)acetamide B130. MS (ESI) m/z: 437.1 [M+1]⁺.

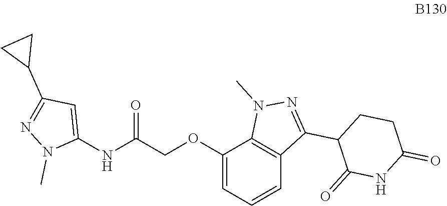 [SMILES: Cn1nc(C2CC2)cc1NC(=O)COc1cccc2c(C3CCC(=O)NC3=O)nn(C)c12]

2-((3-(2,6-Dioxopiperidin-3-yl)-1-methyl-1H-indazol-7-yl)oxy)-N-(4-(p-tolyl-oxy)phenyl)acetamide B131. MS (ESI) m/z: 499.1 [M+1]⁺.

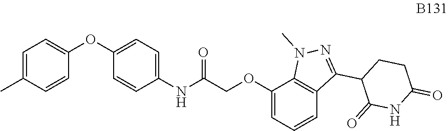 [SMILES: Cc1ccc(Oc2ccc(NC(=O)COc3cccc4c(C5CCC(=O)NC5=O)nn(C)c34)cc2)cc1]

2-((3-(2,6-Dioxopiperidin-3-yl)-1-methyl-1H-indazol-7-yl)oxy)-N-((1-hydroxy-cyclopropyl)methyl)acetamide B132. MS (ESI) m/z: 387.1 [M+1]⁺.

2-((3-(2,6-Dioxopiperidin-3-yl)-1-methyl-1H-indazol-7-yl)oxy)-N-((5-methyl-isoxazol-3-yl)methyl)acetamide B133. MS (ESI) m/z: 412.1 [M+1]⁺.

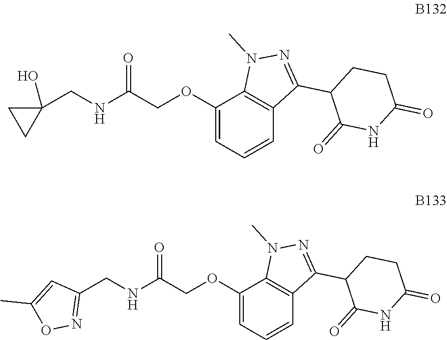 [SMILES: Cn1nc(C2CCC(=O)NC2=O)c2cccc(OCC(=O)NCC3(O)CC3)c21 ; Cc1cc(CNC(=O)COc2cccc3c(C4CCC(=O)NC4=O)nn(C)c23)no1]

2-((3-(2,6-Dioxopiperidin-3-yl)-1-methyl-1H-indazol-7-yl)oxy)-N-((1-methyl-1H-indol-3-yl)methyl)acetamide B134. MS (ESI) m/z: 460.1 [M+1]⁺.

2-((3-(2,6-Dioxopiperidin-3-yl)-1-methyl-1H-indazol-7-yl)oxy)-N-(1-methyl-pyrrolidin-3-yl)acetamide B135. MS (ESI) m/z: 400.1 [M+1]⁺.

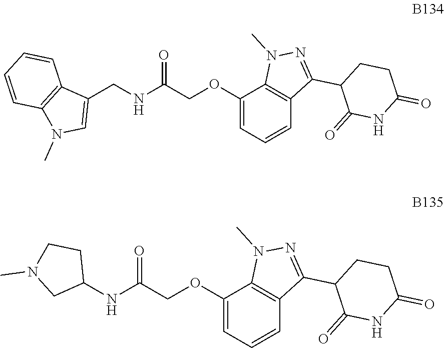 [SMILES: Cn1cc(CNC(=O)COc2cccc3c(C4CCC(=O)NC4=O)nn(C)c23)c2ccccc21 ; CN1CCC(NC(=O)COc2cccc3c(C4CCC(=O)NC4=O)nn(C)c23)C1]

N-(2-(Benzyloxy)ethyl)-2-((3-(2,6-dioxopiperidin-3-yl)-1-methyl-1H-indazol-7-yl)oxy)acetamide B136. MS (ESI) m/z: 451.1 [M+1]⁺.

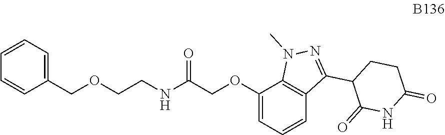 [SMILES: Cn1nc(C2CCC(=O)NC2=O)c2cccc(OCC(=O)NCCOCc3ccccc3)c21]

N-(1-Benzylpyrrolidin-3-yl)-2-((3-(2,6-dioxopiperidin-3-yl)-1-methyl-1H-indazol-7-yl)oxy)acetamide B137. MS (ESI) m/z: 476.2 [M+1]⁺.

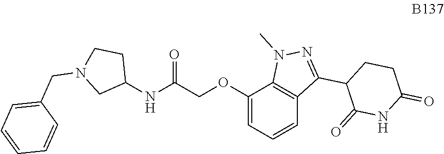 [SMILES: Cn1nc(C2CCC(=O)NC2=O)c2cccc(OCC(=O)NC3CCN(Cc4ccccc4)C3)c21]

2-((3-(2,6-Dioxopiperidin-3-yl)-1-methyl-1H-indazol-7-yl)oxy)-N-(4-isopropyl-benzyl)acetamide B138. MS (ESI) m/z: 449.2 [M+1]⁺.

2-((3-(2,6-Dioxopiperidin-3-yl)-1-methyl-1H-indazol-7-yl)oxy)-N-(tetrahydro-furan-3-yl)acetamide B139. MS (ESI) m/z: 387.2 [M+1]⁺.

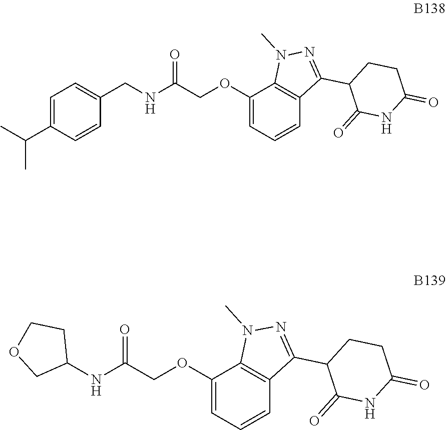 [SMILES: CC(C)c1ccc(CNC(=O)COc2cccc3c(C4CCC(=O)NC4=O)nn(C)c23)cc1 ; Cn1nc(C2CCC(=O)NC2=O)c2cccc(OCC(=O)NC3CCOC3)c21]

2-((3-(2,6-Dioxopiperidin-3-yl)-1-methyl-1H-indazol-7-yl)oxy)-N-(1-methyl-azetidin-3-yl)acetamide B140. MS (ESI) m/z: 386.2 [M+1]⁺.

2-((3-(2,6-Dioxopiperidin-3-yl)-1-methyl-1H-indazol-7-yl)oxy)-N-(1-methyl-1H-pyrazol-5-yl)acetamide B141. MS (ESI) m/z: 397.2 [M+1]⁺.

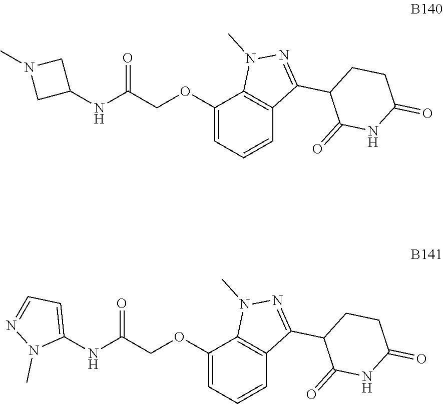 [SMILES: CN1CC(NC(=O)COc2cccc3c(C4CCC(=O)NC4=O)nn(C)c23)C1 ; Cn1nccc1NC(=O)COc1cccc2c(C3CCC(=O)NC3=O)nn(C)c12]

2-((3-(2,6-Dioxopiperidin-3-yl)-1-methyl-1H-indazol-7-yl)oxy)-N-((3-methyl-1,2,4-oxadiazol-5-yl)methyl)acetamide B142. MS (ESI) m/z: 413.2 [M+1]⁺.

2-((3-(2,6-Dioxopiperidin-3-yl)-1-methyl-1H-indazol-7-yl)oxy)-N-(4-sulfamoyl-phenethyl)acetamide B143. MS (ESI) m/z: 500.2 [M+1]⁺.

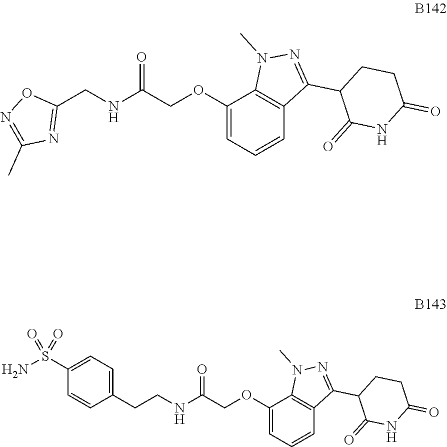 [SMILES: Cc1noc(CNC(=O)COc2cccc3c(C4CCC(=O)NC4=O)nn(C)c23)n1 ; Cn1nc(C2CCC(=O)NC2=O)c2cccc(OCC(=O)NCCc3ccc(S(N)(=O)=O)cc3)c21]

2-((3-(2,6-Dioxopiperidin-3-yl)-1-methyl-1H-indazol-7-yl)oxy)-N-((tetrahydro-furan-2-yl)methyl)acetamide B144. MS (ESI) m/z: 401.2 [M+1]⁺.

2-((3-(2,6-Dioxopiperidin-3-yl)-1-methyl-1H-indazol-7-yl)oxy)-N-((1-methyl-1H-pyrazol-3-yl)methyl)acetamide B145. MS (ESI) m/z: 411.2 [M+1]⁺.

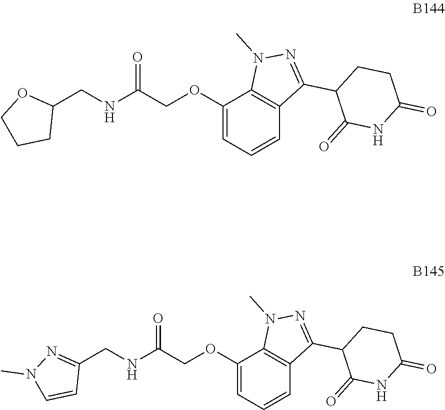 [SMILES: Cn1nc(C2CCC(=O)NC2=O)c2cccc(OCC(=O)NCC3CCCO3)c21 ; Cn1ccc(CNC(=O)COc2cccc3c(C4CCC(=O)NC4=O)nn(C)c23)n1]

2-((3-(2,6-Dioxopiperidin-3-yl)-1-methyl-1H-indazol-7-yl)oxy)-N-(1-propyl-piperidin-4-yl)acetamide B146. MS (ESI) m/z: 442.2 [M+1]⁺.

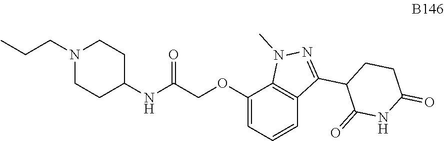 [SMILES: CCCN1CCC(NC(=O)COc2cccc3c(C4CCC(=O)NC4=O)nn(C)c23)CC1]

N-((3-Cyclohexyl-1,2,4-oxadiazol-5-yl)methyl)-2-((3-(2,6-dioxopiperidin-3-yl)-1-methyl-1H-indazol-7-yl)oxy)acetamide B147. MS (ESI) m/z: 481.2 [M+1]⁺.

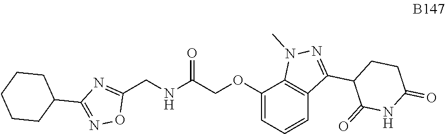 [SMILES: Cn1nc(C2CCC(=O)NC2=O)c2cccc(OCC(=O)NCc3nc(C4CCCCC4)no3)c21]

2-((3-(2,6-Dioxopiperidin-3-yl)-1-methyl-1H-indazol-7-yl)oxy)-N-((5-phenyl-1,2,4-oxadiazol-3-yl)methyl)acetamide B148. MS (ESI) m/z: 475.1 [M+1]⁺.

2-((3-(2,6-Dioxopiperidin-3-yl)-1-methyl-1H-indazol-7-yl)oxy)-N-(3-fluoro-bicyclo[1.1.1]pentan-1-yl)acetamide B149. MS (ESI) m/z: 401.2 [M+1]⁺.

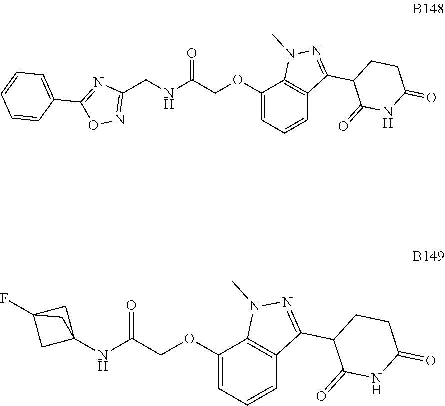 [SMILES: Cn1nc(C2CCC(=O)NC2=O)c2cccc(OCC(=O)NCc3noc(-c4ccccc4)n3)c21 ; Cn1nc(C2CCC(=O)NC2=O)c2cccc(OCC(=O)NC34CC(F)(C3)C4)c21]

2-((3-(2,6-Dioxopiperidin-3-yl)-1-methyl-1H-indazol-7-yl)oxy)-N-(1-isopropyl-1H-pyrazol-4-yl)acetamide B150. MS (ESI) m/z: 425.1 [M+1]⁺.

2-((3-(2,6-Dioxopiperidin-3-yl)-1-methyl-1H-indazol-7-yl)oxy)-N-(2-(p-tolyl-oxy)ethyl)acetamide B151. MS (ESI) m/z: 451.2 [M+1]⁺.

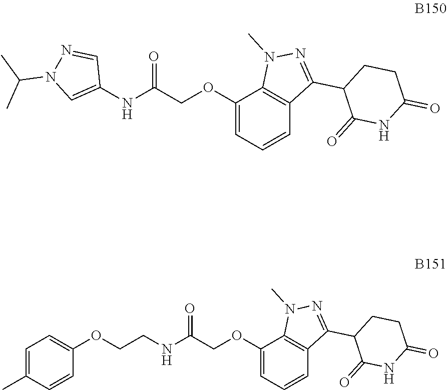 [SMILES: CC(C)n1cc(NC(=O)COc2cccc3c(C4CCC(=O)NC4=O)nn(C)c23)cn1 ; Cc1ccc(OCCNC(=O)COc2cccc3c(C4CCC(=O)NC4=O)nn(C)c23)cc1]

N-(1,1-Dioxidothietan-3-yl)-2-((3-(2,6-dioxopiperidin-3-yl)-1-methyl-1H-indazol-7-yl)oxy)acetamide B152. MS (ESI) m/z: 421.1 [M+1]⁺.

N-(3,5-Dimethoxybenzyl)-2-((3-(2,6-dioxopiperidin-3-yl)-1-methyl-1H-indazol-7-yl)oxy)acetamide B153. MS (ESI) m/z: 467.2 [M+1]⁺.

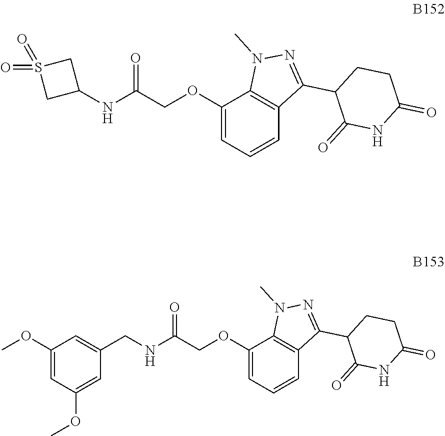 [SMILES: Cn1nc(C2CCC(=O)NC2=O)c2cccc(OCC(=O)NC3CS(=O)(=O)C3)c21 ; COc1cc(CNC(=O)COc2cccc3c(C4CCC(=O)NC4=O)nn(C)c23)cc(OC)c1]

N-(2,5-Difluorobenzyl)-2-((3-(2,6-dioxopiperidin-3-yl)-1-methyl-1H-indazol-7-yl)oxy)acetamide B154. MS (ESI) m/z: 443.1 [M+1]⁺.

2-((3-(2,6-Dioxopiperidin-3-yl)-1-methyl-1H-indazol-7-yl)oxy)-N-((1-methyl-pyrrolidin-3-yl)methyl)acetamide B155. MS (ESI) m/z: 414.1 [M+1]⁺.

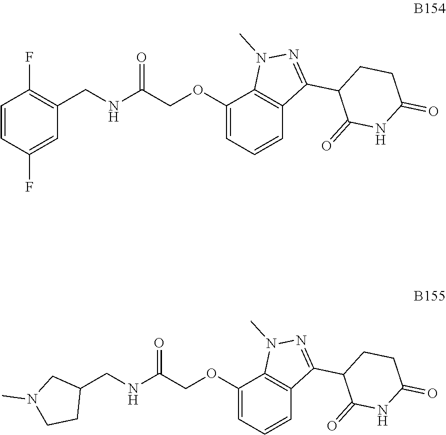 [SMILES: Cn1nc(C2CCC(=O)NC2=O)c2cccc(OCC(=O)NCc3cc(F)ccc3F)c21 ; CN1CCC(CNC(=O)COc2cccc3c(C4CCC(=O)NC4=O)nn(C)c23)C1]

N-((4-Benzylmorpholin-2-yl)methyl)-2-((3-(2,6-dioxopiperidin-3-yl)-1-methyl-1H-indazol-7-yl)oxy)acetamide B156. MS (ESI) m/z: 506.2 [M+1]⁺.

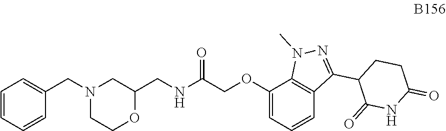 [SMILES: Cn1nc(C2CCC(=O)NC2=O)c2cccc(OCC(=O)NCC3CN(Cc4ccccc4)CCO3)c21]

N-((1,5-Dimethyl-1H-pyrazol-3-yl)methyl)-2-((3-(2,6-dioxopiperidin-3-yl)-1-methyl-1H-indazol-7-yl)oxy)acetamide B157. MS (ESI) m/z: 425.2 [M+1]⁺.

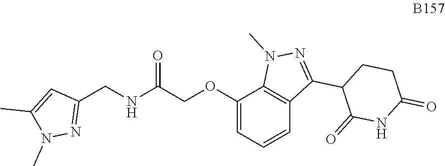 [SMILES: Cc1cc(CNC(=O)COc2cccc3c(C4CCC(=O)NC4=O)nn(C)c23)nn1C]

2-((3-(2,6-Dioxopiperidin-3-yl)-1-methyl-1H-indazol-7-yl)oxy)-N-neopentyl-acetamide B158. MS (ESI) m/z: 387.1 [M+1]⁺.

2-((3-(2,6-Dioxopiperidin-3-yl)-1-methyl-1H-indazol-7-yl)oxy)-N-(3-methyl-1H-pyrazol-5-yl)acetamide B159. MS (ESI) m/z: 397.1 [M+1]⁺.

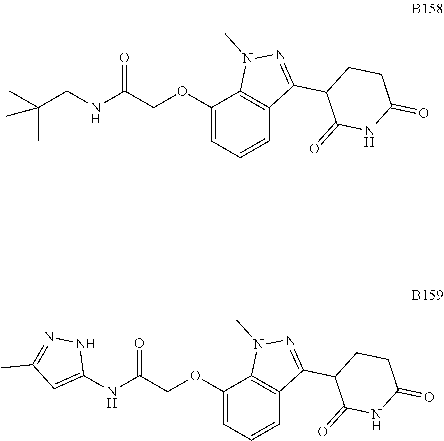 [SMILES: Cn1nc(C2CCC(=O)NC2=O)c2cccc(OCC(=O)NCC(C)(C)C)c21 ; Cc1cc(NC(=O)COc2cccc3c(C4CCC(=O)NC4=O)nn(C)c23)[nH]n1]

2-((3-(2,6-Dioxopiperidin-3-yl)-1-methyl-1H-indazol-7-yl)oxy)-N-(4-(morpholinomethyl)benzyl)acetamide B160. MS (ESI) m/z: 506.2 [M+1]⁺.

2-((3-(2,6-Dioxopiperidin-3-yl)-1-methyl-1H-indazol-7-yl)oxy)-N-(tetrahydro-2H-pyran-4-yl)acetamide B161. MS (ESI) m/z: 401.1 [M+1]⁺.

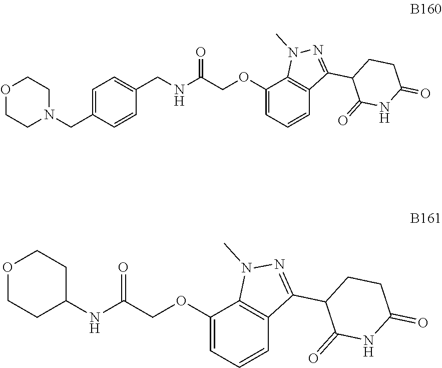 [SMILES: Cn1nc(C2CCC(=O)NC2=O)c2cccc(OCC(=O)NCc3ccc(CN4CCOCC4)cc3)c21 ; Cn1nc(C2CCC(=O)NC2=O)c2cccc(OCC(=O)NC3CCOCC3)c21]

N-(3,5-Dichlorobenzyl)-2-((3-(2,6-dioxopiperidin-3-yl)-1-methyl-1H-indazol-7-yl)oxy)acetamide B162. MS (ESI) m/z: 475.1 [M+1]⁺.

N-(3,5-Difluorobenzyl)-2-((3-(2,6-dioxopiperidin-3-yl)-1-methyl-1H-indazol-7-yl)oxy)acetamide B163. MS (ESI) m/z: 443.1 [M+1]⁺.

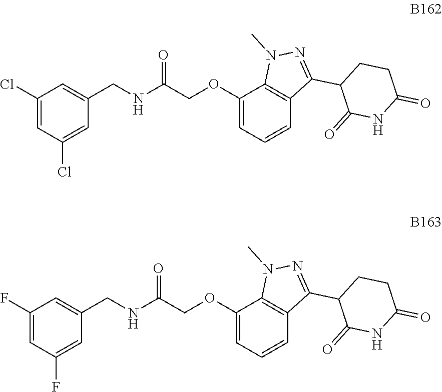 [SMILES: Cn1nc(C2CCC(=O)NC2=O)c2cccc(OCC(=O)NCc3cc(Cl)cc(Cl)c3)c21 ; Cn1nc(C2CCC(=O)NC2=O)c2cccc(OCC(=O)NCc3cc(F)cc(F)c3)c21]

2-((3-(2,6-Dioxopiperidin-3-yl)-1-methyl-1H-indazol-7-yl)oxy)-N-(oxetan-2-yl-methyl)acetamide B164. MS (ESI) m/z: 387.1 [M+1]⁺.

N-(2-(Cyclopentyloxy)ethyl)-2-((3-(2,6-dioxopiperidin-3-yl)-1-methyl-1H-indazol-7-yl)oxy)acetamide B165. MS (ESI) m/z: 429.2 [M+1]⁺.

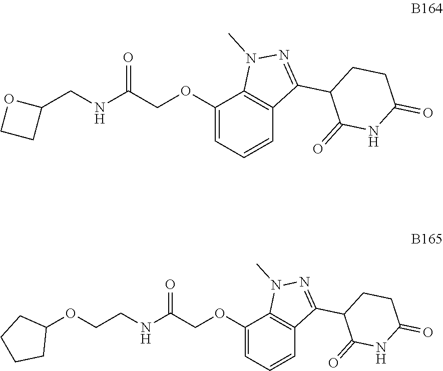 [SMILES: Cn1nc(C2CCC(=O)NC2=O)c2cccc(OCC(=O)NCC3CCO3)c21 ; Cn1nc(C2CCC(=O)NC2=O)c2cccc(OCC(=O)NCCOC3CCCC3)c21]

N-(Cyclopentylmethyl)-2-((3-(2,6-dioxopiperidin-3-yl)-1-methyl-1H-indazol-7-yl)oxy)acetamide B166. MS (ESI) m/z: 399.2 [M+1]⁺.

N-Cyclobutyl-2-((3-(2,6-dioxopiperidin-3-yl)-1-methyl-1H-indazol-7-yl)oxy)-acetamide B167. MS (ESI) m/z: 371.1 [M+1]⁺.

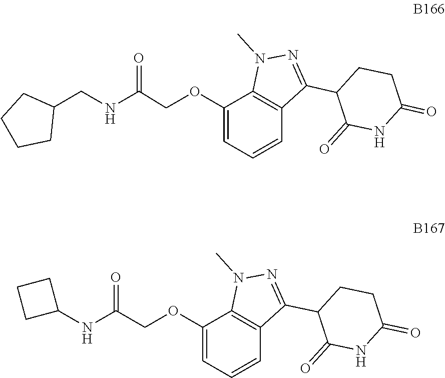 [SMILES: Cn1nc(C2CCC(=O)NC2=O)c2cccc(OCC(=O)NCC3CCCC3)c21 ; Cn1nc(C2CCC(=O)NC2=O)c2cccc(OCC(=O)NC3CCC3)c21]

2-((3-(2,6-Dioxopiperidin-3-yl)-1-methyl-1H-indazol-7-yl)oxy)-N-(thiazol-2-ylmethyl)acetamide B168. MS (ESI) m/z: 414.1 [M+1]⁺.

N-(4-(1H-Pyrazol-1-yl)pyridin-2-yl)-2-((3-(2,6-dioxopiperidin-3-yl)-1-methyl-1H-indazol-7-yl)oxy)acetamide B169. MS (ESI) m/z: 460.2 [M+1]⁺.

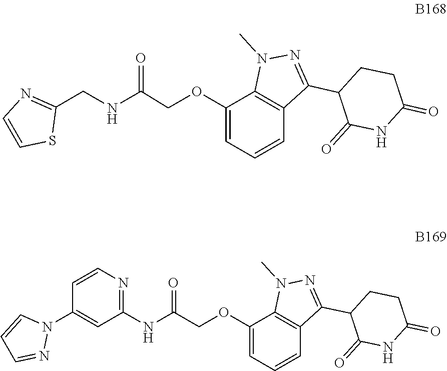 [SMILES: Cn1nc(C2CCC(=O)NC2=O)c2cccc(OCC(=O)NCc3nccs3)c21 ; Cn1nc(C2CCC(=O)NC2=O)c2cccc(OCC(=O)Nc3cc(-n4cccn4)ccn3)c21]

2-((3-(2,6-Dioxopiperidin-3-yl)-1-methyl-1H-indazol-7-yl)oxy)-N-(3-isopropoxy-phenyl)acetamide B170. MS (ESI) m/z: 451.2 [M+1]⁺.

2-((3-(2,6-Dioxopiperidin-3-yl)-1-methyl-1H-indazol-7-yl)oxy)-N-(3-(oxazol-5-yl)phenyl)acetamide B171. MS (ESI) m/z: 460.2 [M+1]⁺.

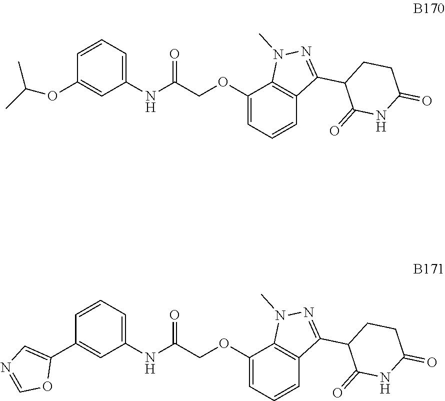 [SMILES: CC(C)Oc1cccc(NC(=O)COc2cccc3c(C4CCC(=O)NC4=O)nn(C)c23)c1 ; Cn1nc(C2CCC(=O)NC2=O)c2cccc(OCC(=O)Nc3cccc(-c4cnco4)c3)c21]

2-((3-(2,6-Dioxopiperidin-3-yl)-1-methyl-1H-indazol-7-yl)oxy)-N-(4-hydroxy-benzyl)acetamide B172. MS (ESI) m/z: 423.2 [M+1]⁺.

2-((3-(2,6-Dioxopiperidin-3-yl)-1-methyl-1H-indazol-7-yl)oxy)-N-(thiophen-2-yl-methyl)acetamide B173. MS (ESI) m/z: 413.2 [M+1]⁺.

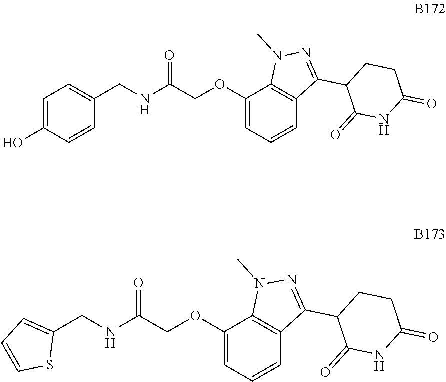 [SMILES: Cn1nc(C2CCC(=O)NC2=O)c2cccc(OCC(=O)NCc3ccc(O)cc3)c21 ; Cn1nc(C2CCC(=O)NC2=O)c2cccc(OCC(=O)NCc3cccs3)c21]

N-Cyclopropyl-2-((3-(2,6-dioxopiperidin-3-yl)-1-methyl-1H-indazol-7-yl)oxy)-acetamide B174. MS (ESI) m/z: 357.2 [M+1]⁺.

N-(3-(Tert-butyl)phenyl)-2-((3-(2,6-dioxopiperidin-3-yl)-1-methyl-1H-indazol-7-yl)oxy)acetamide B175. MS (ESI) m/z: 449.2 [M+1]⁺.

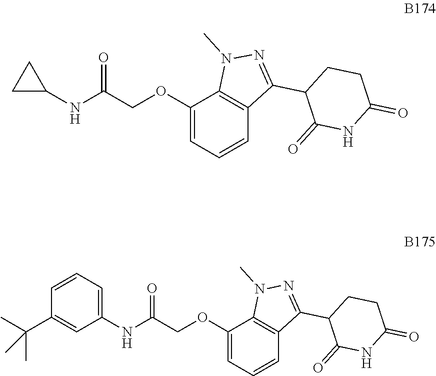 [SMILES: Cn1nc(C2CCC(=O)NC2=O)c2cccc(OCC(=O)NC3CC3)c21 ; Cn1nc(C2CCC(=O)NC2=O)c2cccc(OCC(=O)Nc3cccc(C(C)(C)C)c3)c21]

N-(Cyclopropylmethyl)-2-((3-(2,6-dioxopiperidin-3-yl)-1-methyl-1H-indazol-7-yl)oxy)acetamide B176. MS (ESI) m/z: 373.2 [M+1]⁺.

N-(3-Cyclopropyl-1H-pyrazol-5-yl)-2-((3-(2,6-dioxopiperidin-3-yl)-1-methyl-1H-indazol-7-yl)oxy)acetamide B177. MS (ESI) m/z: 423.2 [M+1]⁺.

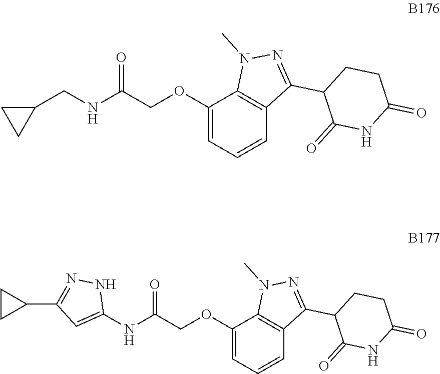 [SMILES: Cn1nc(C2CCC(=O)NC2=O)c2cccc(OCC(=O)NCC3CC3)c21 ; Cn1nc(C2CCC(=O)NC2=O)c2cccc(OCC(=O)Nc3cc(C4CC4)n[nH]3)c21]

N-(3-(1H-Imidazol-1-yl)phenyl)-2-((3-(2,6-dioxopiperidin-3-yl)-1-methyl-1H-indazol-7-yl)oxy)acetamide B178. MS (ESI) m/z: 459.1 [M+1]⁺.

N-Cyclohexyl-2-((3-(2,6-dioxopiperidin-3-yl)-1-methyl-1H-indazol-7-yl)oxy)-acetamide B179. MS (ESI) m/z: 399.2 [M+1]⁺.

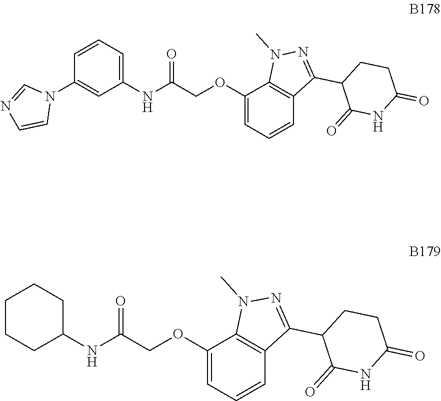 [SMILES: Cn1nc(C2CCC(=O)NC2=O)c2cccc(OCC(=O)Nc3cccc(-n4ccnc4)c3)c21 ; Cn1nc(C2CCC(=O)NC2=O)c2cccc(OCC(=O)NC3CCCCC3)c21]

3-(7-(2-(4-((1,2-Dimethyl-1H-imidazol-5-yl) sulfonyl)piperazin-1-yl)-2-oxo-ethoxy)-1-methyl-1H-indazol-3-yl)piperidine-2,6-dione B203. MS (ESI) m/z: 544.2 [M+1]⁺.

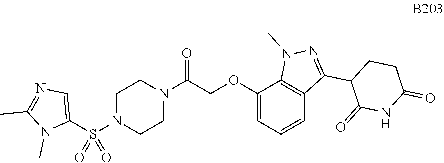 [SMILES: Cc1ncc(S(=O)(=O)N2CCN(C(=O)COc3cccc4c(C5CCC(=O)NC5=O)nn(C)c34)CC2)n1C]

3-(7-(2-(4-((5-(5-Chloro-1,2,4-thiadiazol-3-yl)thiophen-2-yl) sulfonyl)piperazin-1-yl)-2-oxoethoxy)-1-methyl-1H-indazol-3-yl)piperidine-2,6-dione B205. MS (ESI) m/z: 650 [M+1]⁺.

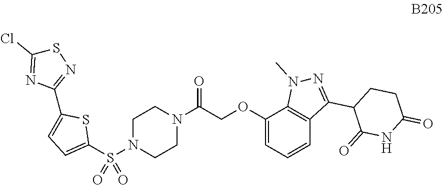 [SMILES: Cn1nc(C2CCC(=O)NC2=O)c2cccc(OCC(=O)N3CCN(S(=O)(=O)c4ccc(-c5nsc(Cl)n5)s4)CC3)c21]

4-Amino-5-(4-(2-((3-(2,6-dioxopiperidin-3-yl)-1-methyl-1H-indazol-7-yl)oxy)-acetyl)piperazine-1-carbonyl)-N-isopropylisothiazole-3-carboxamide B206. MS (ESI) m/z: 597.3 [M+1]⁺.

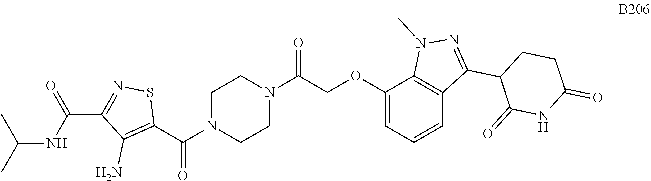 [SMILES: CC(C)NC(=O)c1nsc(C(=O)N2CCN(C(=O)COc3cccc4c(C5CCC(=O)NC5=O)nn(C)c34)CC2)c1N]

3-(1-Methyl-7-(2-(4-((1-methyl-1H-imidazol-4-yl) sulfonyl)piperazin-1-yl)-2-oxo-ethoxy)-1H-indazol-3-yl)piperidine-2,6-dione B208. MS (ESI) m/z: 530.2 [M+1]⁺.

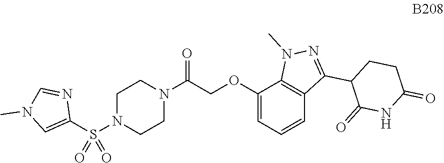 [SMILES: Cn1cnc(S(=O)(=O)N2CCN(C(=O)COc3cccc4c(C5CCC(=O)NC5=O)nn(C)c34)CC2)c1]

4-Amino-5-(4-(2-((3-(2,6-dioxopiperidin-3-yl)-1-methyl-1H-indazol-7-yl)oxy)-acetyl)piperazine-1-carbonyl)-N-ethylisothiazole-3-carboxamide B209. MS (ESI) m/z: 583.2 [M+1]⁺.

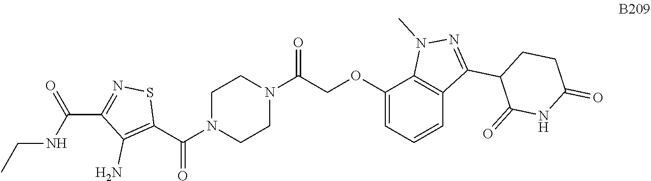 [SMILES: CCNC(=O)c1nsc(C(=O)N2CCN(C(=O)COc3cccc4c(C5CCC(=O)NC5=O)nn(C)c34)CC2)c1N]

3-(1-Methyl-7-(2-oxo-2-(4-((1,3,5-trimethyl-1H-pyrazol-4-yl) sulfonyl)piperazin-1-yl)ethoxy)-1H-indazol-3-yl)piperidine-2,6-dione B210. MS (ESI) m/z: 558.2 [M+1]⁺.

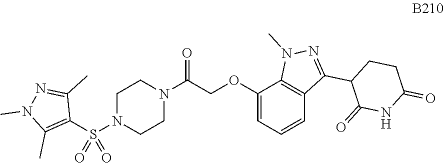 [SMILES: Cc1nn(C)c(C)c1S(=O)(=O)N1CCN(C(=O)COc2cccc3c(C4CCC(=O)NC4=O)nn(C)c23)CC1]

3-(1-Methyl-7-(2-oxo-2-(4-((2,2,4,6,7-pentamethyl-2,3-dihydrobenzofuran-5-yl)-sulfonyl)piperazin-1-yl)ethoxy)-1H-indazol-3-yl)piperidine-2,6-dione B211. MS (ESI) m/z: 638.3 [M+1]⁺.

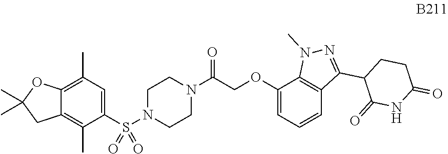 [SMILES: Cc1cc(S(=O)(=O)N2CCN(C(=O)COc3cccc4c(C5CCC(=O)NC5=O)nn(C)c34)CC2)c(C)c2c1OC(C)(C)C2]

3-(7-(2-(4-(2-(Cyclopropylamino) thiazole-5-carbonyl)piperazin-1-yl)-2-oxo-ethoxy)-1-methyl-1H-indazol-3-yl)piperidine-2,6-dione B212. MS (ESI) m/z: 552.2 [M+1]⁺.

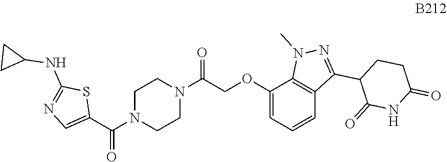 [SMILES: Cn1nc(C2CCC(=O)NC2=O)c2cccc(OCC(=O)N3CCN(C(=O)c4cnc(NC5CC5)s4)CC3)c21]

3-(1-Methyl-7-(2-(4-(3-methylisothiazole-5-carbonyl)piperazin-1-yl)-2-oxo-ethoxy)-1H-indazol-3-yl)piperidine-2,6-dione B213. MS (ESI) m/z: 511.2 [M+1]⁺.

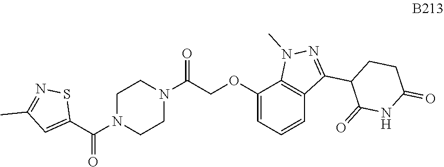 [SMILES: Cc1cc(C(=O)N2CCN(C(=O)COc3cccc4c(C5CCC(=O)NC5=O)nn(C)c34)CC2)sn1]

3-(7-(2-(4-((1-Ethyl-3-methyl-1H-pyrazol-5-yl) sulfonyl)piperazin-1-yl)-2-oxo-ethoxy)-1-methyl-1H-indazol-3-yl)piperidine-2,6-dione B214. MS (ESI) m/z: 558.2 [M+1]⁺.

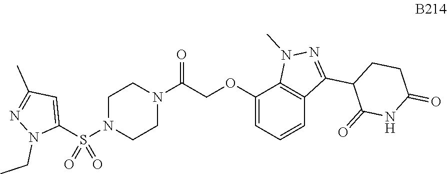 [SMILES: CCn1nc(C)cc1S(=O)(=O)N1CCN(C(=O)COc2cccc3c(C4CCC(=O)NC4=O)nn(C)c23)CC1]

3-(7-(2-(4-((1-Ethyl-1H-pyrazol-4-yl) sulfonyl)piperazin-1-yl)-2-oxoethoxy)-1-methyl-1H-indazol-3-yl)piperidine-2,6-dione B216. MS (ESI) m/z: 544.2 [M+1]⁺.

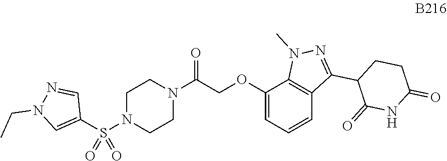 [SMILES: CCn1cc(S(=O)(=O)N2CCN(C(=O)COc3cccc4c(C5CCC(=O)NC5=O)nn(C)c34)CC2)cn1]

3-(7-(2-(4-((Cyclobutylmethyl) sulfonyl)piperazin-1-yl)-2-oxoethoxy)-1-methyl-1H-indazol-3-yl)piperidine-2,6-dione B217. MS (ESI) m/z: 518.2 [M+1]⁺.

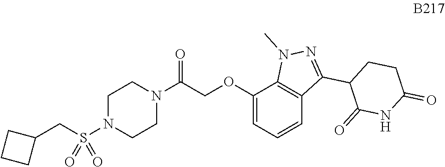 [SMILES: Cn1nc(C2CCC(=O)NC2=O)c2cccc(OCC(=O)N3CCN(S(=O)(=O)CC4CCC4)CC3)c21]

3-(1-Methyl-7-(2-(4-((3-(5-methyl-1,2,4-oxadiazol-3-yl)phenyl) sulfonyl)-piperazin-1-yl)-2-oxoethoxy)-1H-indazol-3-yl)piperidine-2,6-dione B218. MS (ESI) m/z: 608.2 [M+1]⁺.

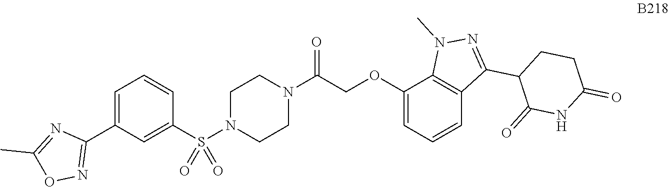 [SMILES: Cc1nc(-c2cccc(S(=O)(=O)N3CCN(C(=O)COc4cccc5c(C6CCC(=O)NC6=O)nn(C)c45)CC3)c2)no1]

3-(7-(2-(4-(6,7-Dihydro-4H-pyrazolo[5,1-c][1,4]oxazine-2-carbonyl)piperazin-1-yl)-2-oxoethoxy)-1-methyl-1H-indazol-3-yl)piperidine-2,6-dione B220. MS (ESI) m/z: 536.3 [M+1]⁺.

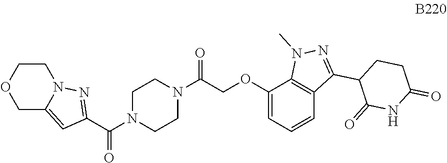 [SMILES: Cn1nc(C2CCC(=O)NC2=O)c2cccc(OCC(=O)N3CCN(C(=O)c4cc5n(n4)CCOC5)CC3)c21]

3-(7-(2-(4-(2,4-Dimethyloxazole-5-carbonyl)piperazin-1-yl)-2-oxoethoxy)-1-methyl-1H-indazol-3-yl)piperidine-2,6-dione B221. MS (ESI) m/z: 509.2 [M+1]⁺.

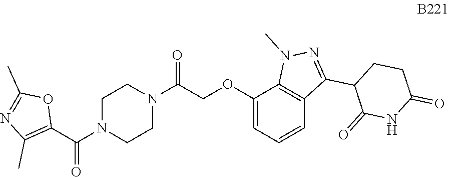 [SMILES: Cc1nc(C)c(C(=O)N2CCN(C(=O)COc3cccc4c(C5CCC(=O)NC5=O)nn(C)c34)CC2)o1]

3-(1-Methyl-7-(2-oxo-2-(4-((4-(trifluoromethoxy)phenyl) sulfonyl)piperazin-1-yl)ethoxy)-1H-indazol-3-yl)piperidine-2,6-dione B222. MS (ESI) m/z: 610.2 [M+1]⁺.

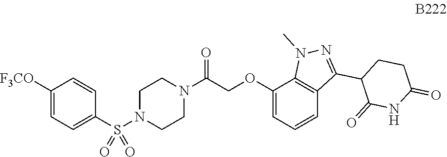 [SMILES: Cn1nc(C2CCC(=O)NC2=O)c2cccc(OCC(=O)N3CCN(S(=O)(=O)c4ccc(OC(F)(F)F)cc4)CC3)c21]

3-(1-Methyl-7-(2-(4-(4-methylthiazole-2-carbonyl)piperazin-1-yl)-2-oxoethoxy)-1H-indazol-3-yl)piperidine-2,6-dione B223. MS (ESI) m/z: 511.2 [M+1]⁺.

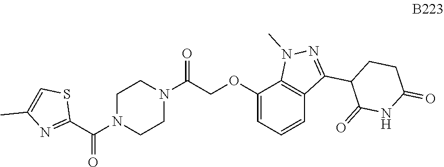 [SMILES: Cc1csc(C(=O)N2CCN(C(=O)COc3cccc4c(C5CCC(=O)NC5=O)nn(C)c34)CC2)n1]

5-(4-(2-((3-(2,6-Dioxopiperidin-3-yl)-1-methyl-1H-indazol-7-yl)oxy)acetyl)-piperazine-1-carbonyl)-1H-pyrrole-3-carbonitrile B224. MS (ESI) m/z: 504.3 [M+1]⁺.

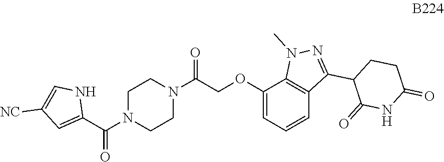 [SMILES: Cn1nc(C2CCC(=O)NC2=O)c2cccc(OCC(=O)N3CCN(C(=O)c4cc(C#N)c[nH]4)CC3)c21]

3-(1-Methyl-7-(2-(4-(2-methyl-3H-pyrazolo[1,5-b][1,2,4]triazole-7-carbonyl)-piperazin-1-yl)-2-oxoethoxy)-1H-indazol-3-yl)piperidine-2,6-dione B225. MS (ESI) m/z: 534.3 [M+1]⁺.

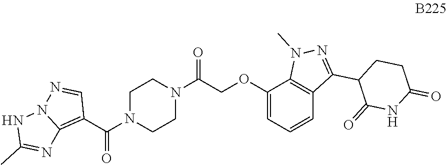 [SMILES: Cc1nc2c(C(=O)N3CCN(C(=O)COc4cccc5c(C6CCC(=O)NC6=O)nn(C)c45)CC3)cnn2[nH]1]

Methyl 5-(4-(2-((3-(2,6-dioxopiperidin-3-yl)-1-methyl-1H-indazol-7-yl)oxy)-acetyl)piperazine-1-carbonyl)-1-methyl-1H-pyrazole-3-carboxylate B226. MS (ESI) m/z: 552.2 [M+1]⁺.

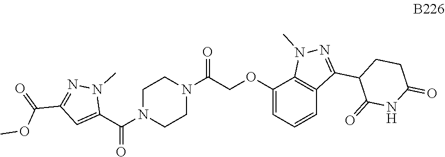 [SMILES: COC(=O)c1cc(C(=O)N2CCN(C(=O)COc3cccc4c(C5CCC(=O)NC5=O)nn(C)c34)CC2)n(C)n1]

Ethyl 5-(4-(2-((3-(2,6-dioxopiperidin-3-yl)-1-methyl-1H-indazol-7-yl)oxy)-acetyl)piperazine-1-carbonyl)-1H-pyrrole-2-carboxylate B227. MS (ESI) m/z: 551.2 [M+1]⁺.

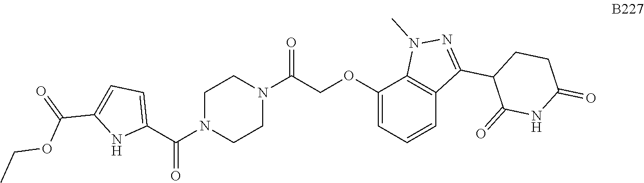 [SMILES: CCOC(=O)c1ccc(C(=O)N2CCN(C(=O)COc3cccc4c(C5CCC(=O)NC5=O)nn(C)c34)CC2)[nH]1]

3-(7-(2-(4-(2,5-Dimethyloxazole-4-carbonyl)piperazin-1-yl)-2-oxoethoxy)-1-methyl-1H-indazol-3-yl)piperidine-2,6-dione B228. MS (ESI) m/z: 509.3 [M+1]⁺.

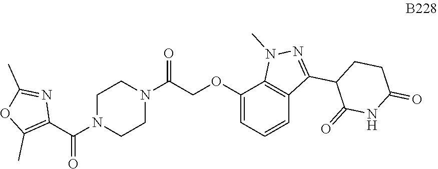 [SMILES: Cc1nc(C(=O)N2CCN(C(=O)COc3cccc4c(C5CCC(=O)NC5=O)nn(C)c34)CC2)c(C)o1]

3-(1-Methyl-7-(2-oxo-2-(4-(2,4,5,6-tetrahydrocyclopenta[c]pyrrole-1-carbonyl)-piperazin-1-yl)ethoxy)-1H-indazol-3-yl)piperidine-2,6-dione B229. MS (ESI) m/z: 519.2 [M+1]⁺.

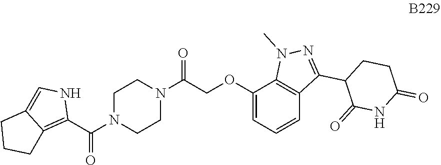 [SMILES: Cn1nc(C2CCC(=O)NC2=O)c2cccc(OCC(=O)N3CCN(C(=O)c4[nH]cc5c4CCC5)CC3)c21]

3-(1-Methyl-7-(2-oxo-2-(4-((3-oxo-3,4-dihydro-2H-benzo[b][1,4]oxazin-6-yl)-sulfonyl)piperazin-1-yl)ethoxy)-1H-indazol-3-yl)piperidine-2,6-dione B230. MS (ESI) m/z: 597.2 [M+1]⁺.

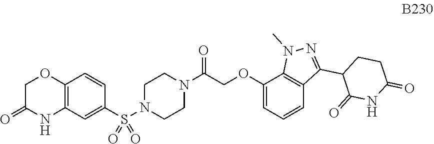 [SMILES: Cn1nc(C2CCC(=O)NC2=O)c2cccc(OCC(=O)N3CCN(S(=O)(=O)c4ccc5c(c4)NC(=O)CO5)CC3)c21]

3-(1-Methyl-7-(2-oxo-2-(4-((2-oxoindolin-6-yl) sulfonyl)piperazin-1-yl)ethoxy)-1H-indazol-3-yl)piperidine-2,6-dione B231. MS (ESI) m/z: 581.2 [M+1]⁺.

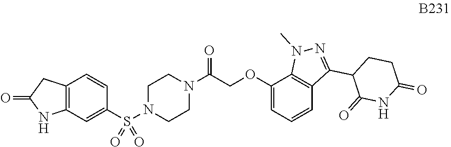 [SMILES: Cn1nc(C2CCC(=O)NC2=O)c2cccc(OCC(=O)N3CCN(S(=O)(=O)c4ccc5c(c4)NC(=O)C5)CC3)c21]

3-(7-(2-(4-((2-Chloro-5-methylpyridin-3-yl) sulfonyl)piperazin-1-yl)-2-oxo-ethoxy)-1-methyl-1H-indazol-3-yl)piperidine-2,6-dione B232. MS (ESI) m/z: 575.2 [M+1]⁺.

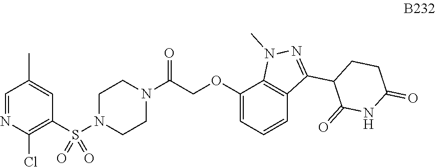 [SMILES: Cc1cnc(Cl)c(S(=O)(=O)N2CCN(C(=O)COc3cccc4c(C5CCC(=O)NC5=O)nn(C)c34)CC2)c1]

4-((4-(2-((3-(2,6-Dioxopiperidin-3-yl)-1-methyl-1H-indazol-7-yl)oxy)acetyl)-piperazin-1-yl) sulfonyl)benzamide B234. MS (ESI) m/z: 569.2 [M+1]⁺.

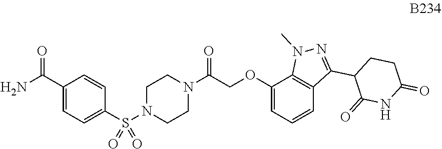 [SMILES: Cn1nc(C2CCC(=O)NC2=O)c2cccc(OCC(=O)N3CCN(S(=O)(=O)c4ccc(C(N)=O)cc4)CC3)c21]

3-(7-(2-(4-((4-(Tert-butyl)phenyl) sulfonyl)piperazin-1-yl)-2-oxoethoxy)-1-methyl-1H-indazol-3-yl)piperidine-2,6-dione B235. MS (ESI) m/z: 582.3 [M+1]⁺.

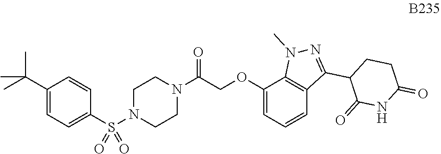 [SMILES: Cn1nc(C2CCC(=O)NC2=O)c2cccc(OCC(=O)N3CCN(S(=O)(=O)c4ccc(C(C)(C)C)cc4)CC3)c21]

Ethyl 4-(4-(2-((3-(2,6-dioxopiperidin-3-yl)-1-methyl-1H-indazol-7-yl)oxy)-acetyl)piperazine-1-carbonyl)-1H-pyrrole-2-carboxylate B236. MS (ESI) m/z: 551.3 [M+1]⁺.

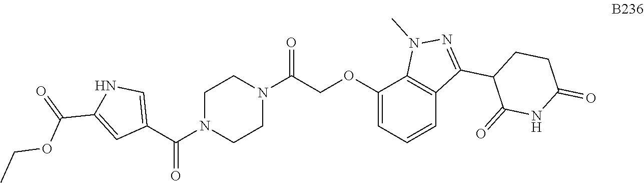 [SMILES: CCOC(=O)c1cc(C(=O)N2CCN(C(=O)COc3cccc4c(C5CCC(=O)NC5=O)nn(C)c34)CC2)c[nH]1]

3-(7-(2-(4-((1,5-Dimethyl-1H-pyrazol-3-yl) sulfonyl)piperazin-1-yl)-2-oxo-ethoxy)-1-methyl-1H-indazol-3-yl)piperidine-2,6-dione B237. MS (ESI) m/z: 544.2 [M+1]⁺.

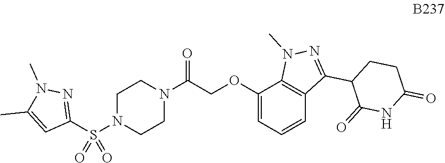 [SMILES: Cc1cc(S(=O)(=O)N2CCN(C(=O)COc3cccc4c(C5CCC(=O)NC5=O)nn(C)c34)CC2)nn1C]

3-(1-Methyl-7-(2-(4-((1-methyl-1H-pyrazol-4-yl) sulfonyl)piperazin-1-yl)-2-oxo-ethoxy)-1H-indazol-3-yl)piperidine-2,6-dione B238. MS (ESI) m/z: 530.1 [M+1]⁺.

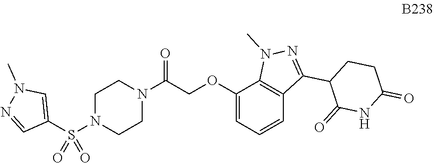 [SMILES: Cn1cc(S(=O)(=O)N2CCN(C(=O)COc3cccc4c(C5CCC(=O)NC5=O)nn(C)c34)CC2)cn1]

3-(1-Methyl-7-(2-(4-(5-methylthiazole-2-carbonyl)piperazin-1-yl)-2-oxoethoxy)-1H-indazol-3-yl)piperidine-2,6-dione B239. MS (ESI) m/z: 511.1 [M+1]⁺.

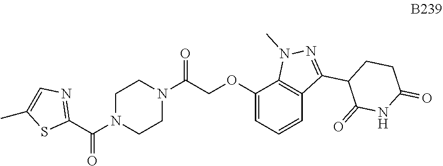 [SMILES: Cc1cnc(C(=O)N2CCN(C(=O)COc3cccc4c(C5CCC(=O)NC5=O)nn(C)c34)CC2)s1]

3-(7-(2-(4-((4-Fluorophenyl) sulfonyl)piperazin-1-yl)-2-oxoethoxy)-1-methyl-1H-indazol-3-yl)piperidine-2,6-dione B240. MS (ESI) m/z: 544.2 [M+1]⁺.

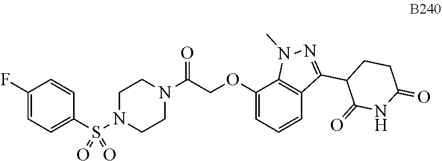 [SMILES: Cn1nc(C2CCC(=O)NC2=O)c2cccc(OCC(=O)N3CCN(S(=O)(=O)c4ccc(F)cc4)CC3)c21]

3-(1-Methyl-7-(2-oxo-2-(4-(thiophen-2-ylsulfonyl)piperazin-1-yl)ethoxy)-1H-indazol-3-yl)piperidine-2,6-dione B241. MS (ESI) m/z: 532.2 [M+1]⁺.

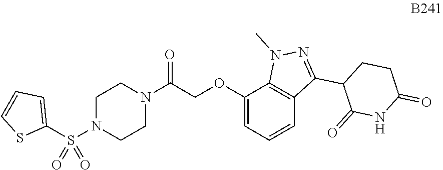 [SMILES: Cn1nc(C2CCC(=O)NC2=O)c2cccc(OCC(=O)N3CCN(S(=O)(=O)c4cccs4)CC3)c21]

5-(4-(2-((3-(2,6-Dioxopiperidin-3-yl)-1-methyl-1H-indazol-7-yl)oxy)acetyl)-piperazine-1-carbonyl) thiazole-2-carbonitrile B243. MS (ESI) m/z: 522.2 [M+1]⁺.

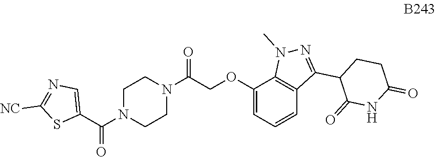 [SMILES: Cn1nc(C2CCC(=O)NC2=O)c2cccc(OCC(=O)N3CCN(C(=O)c4cnc(C#N)s4)CC3)c21]

3-(7-((1-(4-Amino-1-methyl-1H-pyrazole-3-carbonyl)piperidin-4-yl)oxy)-1-methyl-1H-indazol-3-yl)piperidine-2,6-dione C002. MS (ESI) m/z: 466.3 [M+1]⁺.

3-(1-Methyl-7-((1-(2-(4-oxo-2-thioxothiazolidin-3-yl)acetyl)piperidin-4-yl)oxy)-1H-indazol-3-yl)piperidine-2,6-dione C003. MS (ESI) m/z: 516.2 [M+1]⁺.

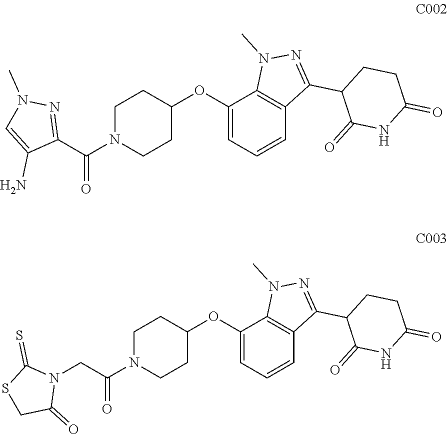 [SMILES: Cn1cc(N)c(C(=O)N2CCC(Oc3cccc4c(C5CCC(=O)NC5=O)nn(C)c34)CC2)n1 ; Cn1nc(C2CCC(=O)NC2=O)c2cccc(OC3CCN(C(=O)CN4C(=O)CSC4=S)CC3)c21]

3-(1-Methyl-7-((1-(4-oxo-1,4-dihydropyrrolo[1,2-b]pyridazine-3-carbonyl)-piperidin-4-yl)oxy)-1H-indazol-3-yl)piperidine-2,6-dione C004. MS (ESI) m/z: 503.2 [M+1]⁺.

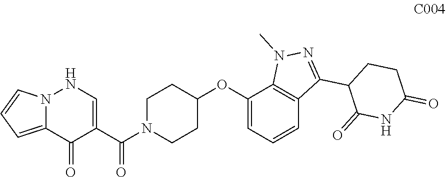 [SMILES: Cn1nc(C2CCC(=O)NC2=O)c2cccc(OC3CCN(C(=O)c4c[nH]n5cccc5c4=O)CC3)c21]

3-(1-Methyl-7-((1-(3-(4-(trifluoromethyl)phenyl) propioloyl)piperidin-4-yl)oxy)-1H-indazol-3-yl)piperidine-2,6-dione C005. MS (ESI) m/z: 479.2 [M+1]⁺.

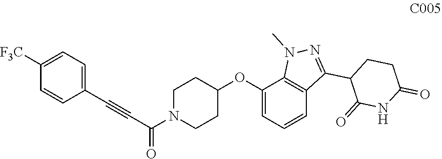 [SMILES: Cn1nc(C2CCC(=O)NC2=O)c2cccc(OC3CCN(C(=O)C#Cc4ccc(C(F)(F)F)cc4)CC3)c21]

5-(4-((3-(2,6-Dioxopiperidin-3-yl)-1-methyl-1H-indazol-7-yl)oxy) piperidine-1-carbonyl)-1H-pyrrole-2-carboxamide C006. MS (ESI) m/z: 479.2 [M+1]⁺.

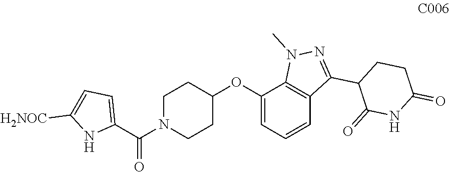 [SMILES: Cn1nc(C2CCC(=O)NC2=O)c2cccc(OC3CCN(C(=O)c4ccc(C(N)=O)[nH]4)CC3)c21]

3-(7-((1-(4-Amino-1-methyl-1H-pyrazole-5-carbonyl)piperidin-4-yl)oxy)-1-methyl-1H-indazol-3-yl)piperidine-2,6-dione C007. MS (ESI) m/z: 466.2 [M+1]⁺.

3-(1-Methyl-7-((1-(methylglycyl)piperidin-4-yl)oxy)-1H-indazol-3-yl)piperidine-2,6-dione C008. MS (ESI) m/z: 414.2 [M+1]⁺.

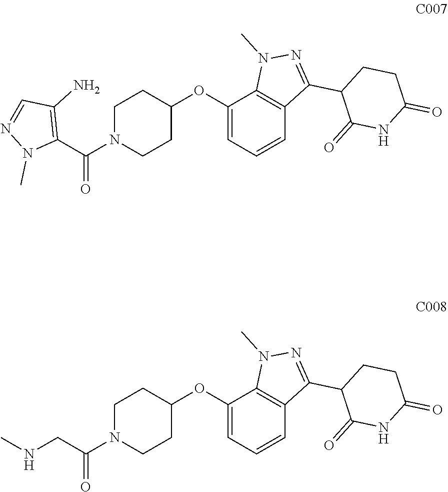 [SMILES: Cn1ncc(N)c1C(=O)N1CCC(Oc2cccc3c(C4CCC(=O)NC4=O)nn(C)c23)CC1 ; CNCC(=O)N1CCC(Oc2cccc3c(C4CCC(=O)NC4=O)nn(C)c23)CC1]

2-(4-((3-(2,6-Dioxopiperidin-3-yl)-1-methyl-1H-indazol-7-yl)oxy) piperidine-1-carbonyl)-1H-pyrrole-3-carbonitrile C009. MS (ESI) m/z: 461.2 [M+1]⁺.

3-(7-((1-(Azetidine-3-carbonyl)piperidin-4-yl)oxy)-1-methyl-1H-indazol-3-yl)-piperidine-2,6-dione C010. MS (ESI) m/z: 426.2 [M+1]⁺.

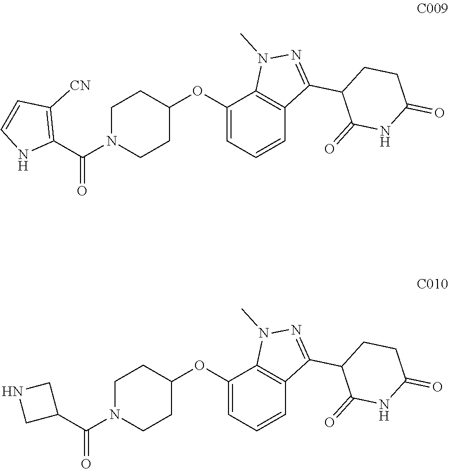 [SMILES: Cn1nc(C2CCC(=O)NC2=O)c2cccc(OC3CCN(C(=O)c4[nH]ccc4C#N)CC3)c21 ; Cn1nc(C2CCC(=O)NC2=O)c2cccc(OC3CCN(C(=O)C4CNC4)CC3)c21]

(E)-3-(1-Methyl-7-((1-(3-(4-(trifluoromethyl)phenyl) acryloyl)piperidin-4-yl)oxy)-1H-indazol-3-yl)piperidine-2,6-dione C011. MS (ESI) m/z: 541.2 [M+1]⁺.

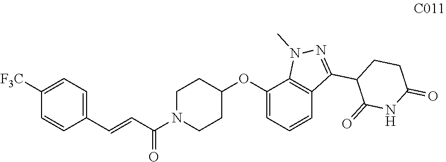 [SMILES: Cn1nc(C2CCC(=O)NC2=O)c2cccc(OC3CCN(C(=O)/C=C/c4ccc(C(F)(F)F)cc4)CC3)c21]

Ethyl 4-(4-((3-(2,6-dioxopiperidin-3-yl)-1-methyl-1H-indazol-7-yl)oxy)-piperidine-1-carbonyl)-1H-pyrrole-2-carboxylate C012. MS (ESI) m/z: 508.2 [M+1]⁺.

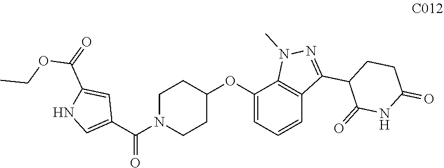 [SMILES: CCOC(=O)c1cc(C(=O)N2CCC(Oc3cccc4c(C5CCC(=O)NC5=O)nn(C)c34)CC2)c[nH]1]

3-(1-Methyl-7-((1-(2-phenylthiazole-4-carbonyl)piperidin-4-yl)oxy)-1H-indazol-3-yl)piperidine-2,6-dione C013. MS (ESI) m/z: 530.2 [M+1]⁺.

3-(7-((1-(5-Methoxypicolinoyl)piperidin-4-yl)oxy)-1-methyl-1H-indazol-3-yl)-piperidine-2,6-dione C014. MS (ESI) m/z: 478.2 [M+1]⁺.

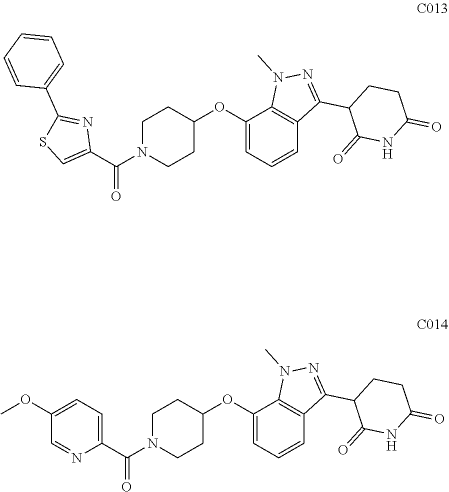 [SMILES: Cn1nc(C2CCC(=O)NC2=O)c2cccc(OC3CCN(C(=O)c4csc(-c5ccccc5)n4)CC3)c21 ; COc1ccc(C(=O)N2CCC(Oc3cccc4c(C5CCC(=O)NC5=O)nn(C)c34)CC2)nc1]

3-(7-((1-(2-((2,4-Difluorophenyl)thio) acetyl)piperidin-4-yl)oxy)-1-methyl-1H-indazol-3-yl)piperidine-2,6-dione C015. MS (ESI) m/z: 529.2 [M+1]⁺.

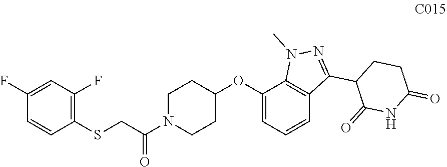 [SMILES: Cn1nc(C2CCC(=O)NC2=O)c2cccc(OC3CCN(C(=O)CSc4ccc(F)cc4F)CC3)c21]

4-(4-((3-(2,6-Dioxopiperidin-3-yl)-1-methyl-1H-indazol-7-yl)oxy) piperidine-1-carbonyl)-3-fluorobenzonitrile C016. MS (ESI) m/z: 490.2 [M+1]⁺.

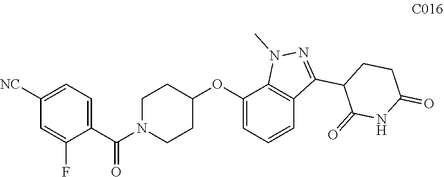 [SMILES: Cn1nc(C2CCC(=O)NC2=O)c2cccc(OC3CCN(C(=O)c4ccc(C#N)cc4F)CC3)c21]

3-(1-Methyl-7-((1-(1-methyl-3-(trifluoromethyl)-1H-pyrazole-4-carbonyl)-piperidin-4-yl)oxy)-1H-indazol-3-yl)piperidine-2,6-dione C017. MS (ESI) m/z: 519.2 [M+1]⁺.

3-(1-Methyl-7-((1-(2-((phenylthio)methyl)benzoyl)piperidin-4-yl)oxy)-1H-indazol-3-yl)piperidine-2,6-dione C018. MS (ESI) m/z: 569.2 [M+1]⁺.

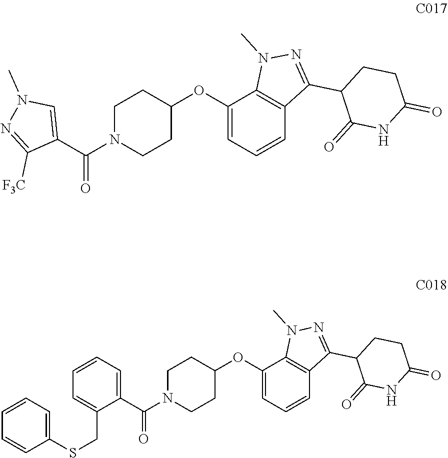 [SMILES: Cn1cc(C(=O)N2CCC(Oc3cccc4c(C5CCC(=O)NC5=O)nn(C)c34)CC2)c(C(F)(F)F)n1 ; Cn1nc(C2CCC(=O)NC2=O)c2cccc(OC3CCN(C(=O)c4ccccc4CSc4ccccc4)CC3)c21]

3-(1-Methyl-7-((1-(1-methyl-5-(trifluoromethyl)-1H-pyrrole-2-carbonyl)-piperidin-4-yl)oxy)-1H-indazol-3-yl)piperidine-2,6-dione C019. MS (ESI) m/z: 518.2 [M+1]⁺.

3-(7-((1-(3-Methoxythiophene-2-carbonyl)piperidin-4-yl)oxy)-1-methyl-1H-indazol-3-yl)piperidine-2,6-dione C020. MS (ESI) m/z: 483.1 [M+1]⁺.

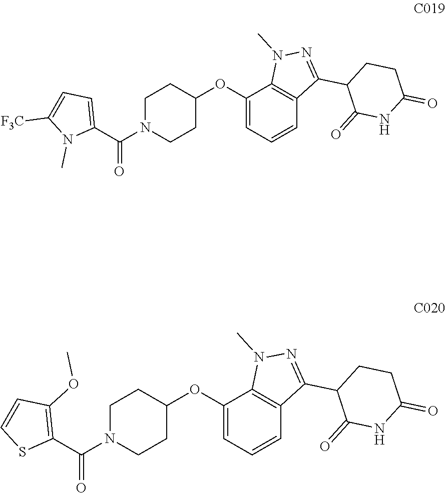 [SMILES: Cn1c(C(=O)N2CCC(Oc3cccc4c(C5CCC(=O)NC5=O)nn(C)c34)CC2)ccc1C(F)(F)F ; COc1ccsc1C(=O)N1CCC(Oc2cccc3c(C4CCC(=O)NC4=O)nn(C)c23)CC1]

3-(1-Methyl-7-((1-(pyrrolo[2,1-b]thiazole-6-carbonyl)piperidin-4-yl)oxy)-1H-indazol-3-yl)piperidine-2,6-dione C021. MS (ESI) m/z: 492.1 [M+1]⁺.

3-(7-((1-(4,5-Dimethylthiophene-3-carbonyl)piperidin-4-yl)oxy)-1-methyl-1H-indazol-3-yl)piperidine-2,6-dione C022. MS (ESI) m/z: 481.2 [M+1]⁺.

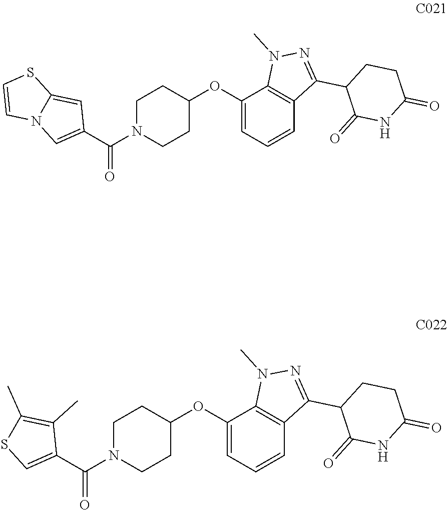 [SMILES: Cn1nc(C2CCC(=O)NC2=O)c2cccc(OC3CCN(C(=O)c4cc5sccn5c4)CC3)c21 ; Cc1scc(C(=O)N2CCC(Oc3cccc4c(C5CCC(=O)NC5=O)nn(C)c34)CC2)c1C]

3-(1-Methyl-7-((1-(1-methyl-1H-pyrrole-2-carbonyl)piperidin-4-yl)oxy)-1H-indazol-3-yl)piperidine-2,6-dione C023. MS (ESI) m/z: 450.2 [M+1]⁺.

3-(7-((1-(2,2-Difluoro-2-(4-fluorophenyl)acetyl)piperidin-4-yl)oxy)-1-methyl-1H-indazol-3-yl)piperidine-2,6-dione C024. MS (ESI) m/z: 515.2 [M+1]⁺.

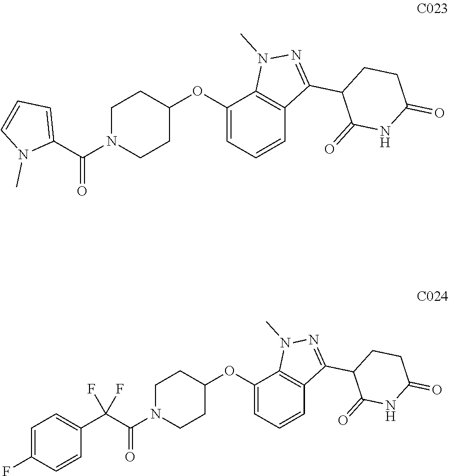 [SMILES: Cn1cccc1C(=O)N1CCC(Oc2cccc3c(C4CCC(=O)NC4=O)nn(C)c23)CC1 ; Cn1nc(C2CCC(=O)NC2=O)c2cccc(OC3CCN(C(=O)C(F)(F)c4ccc(F)cc4)CC3)c21]

3-(7-((1-(2-Cyclobutylacetyl)piperidin-4-yl)oxy)-1-methyl-1H-indazol-3-yl)-piperidine-2,6-dione C025. MS (ESI) m/z: 439.2 [M+1]⁺.

3-(7-((1-(Bicyclo[1.1.1]pentane-1-carbonyl)piperidin-4-yl)oxy)-1-methyl-1H-indazol-3-yl)piperidine-2,6-dione C026. MS (ESI) m/z: 437.2 [M+1]⁺.

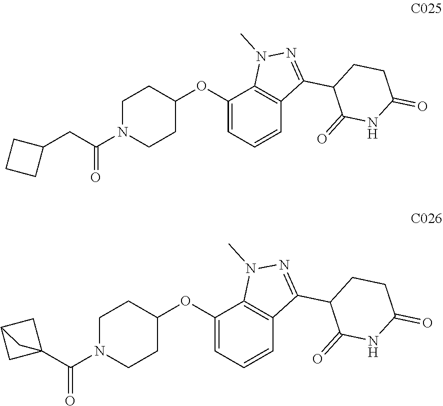 [SMILES: Cn1nc(C2CCC(=O)NC2=O)c2cccc(OC3CCN(C(=O)CC4CCC4)CC3)c21 ; Cn1nc(C2CCC(=O)NC2=O)c2cccc(OC3CCN(C(=O)C45CC(C4)C5)CC3)c21]

3-(7-((1-(4-Isopropoxybenzoyl)piperidin-4-yl)oxy)-1-methyl-1H-indazol-3-yl)-piperidine-2,6-dione C027. MS (ESI) m/z: 505.3 [M+1]⁺.

3-(7-((1-(1,1-Dioxidothietane-3-carbonyl)piperidin-4-yl)oxy)-1-methyl-1H-indazol-3-yl)piperidine-2,6-dione C028. MS (ESI) m/z: 475.1 [M+1]⁺.

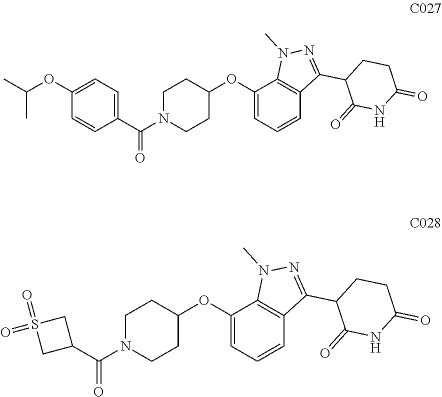 [SMILES: CC(C)Oc1ccc(C(=O)N2CCC(Oc3cccc4c(C5CCC(=O)NC5=O)nn(C)c34)CC2)cc1 ; Cn1nc(C2CCC(=O)NC2=O)c2cccc(OC3CCN(C(=O)C4CS(=O)(=O)C4)CC3)c21]

3-(7-((1-(Imidazo[1,2-a]pyridine-6-carbonyl)piperidin-4-yl)oxy)-1-methyl-1H-indazol-3-yl)piperidine-2,6-dione C029. MS (ESI) m/z: 487.2 [M+1]⁺.

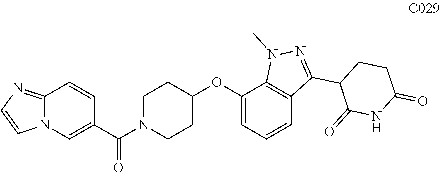 [SMILES: Cn1nc(C2CCC(=O)NC2=O)c2cccc(OC3CCN(C(=O)c4ccc5nccn5c4)CC3)c21]

3-(1-Methyl-7-((1-(4-phenoxybenzoyl)piperidin-4-yl)oxy)-1H-indazol-3-yl)-piperidine-2,6-dione C030. MS (ESI) m/z: 539.2 [M+1]⁺.

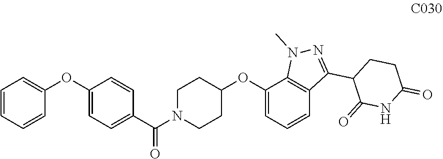 [SMILES: Cn1nc(C2CCC(=O)NC2=O)c2cccc(OC3CCN(C(=O)c4ccc(Oc5ccccc5)cc4)CC3)c21]

3-(1-Methyl-7-((1-(4-oxo-4H-chromene-2-carbonyl)piperidin-4-yl)oxy)-1H-indazol-3-yl)piperidine-2,6-dione C031. MS (ESI) m/z: 515.2 [M+1]⁺.

3-(1-Methyl-7-((1-(3-phenylpropioloyl)piperidin-4-yl)oxy)-1H-indazol-3-yl)-piperidine-2,6-dione C032. MS (ESI) m/z: 471.2 [M+1]⁺.

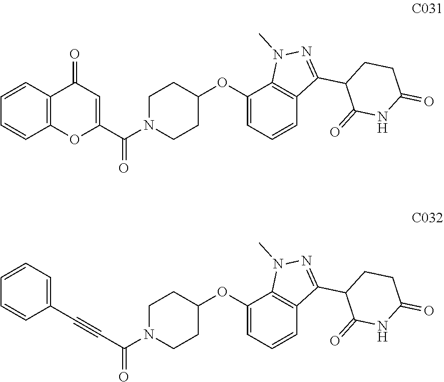 [SMILES: Cn1nc(C2CCC(=O)NC2=O)c2cccc(OC3CCN(C(=O)c4cc(=O)c5ccccc5o4)CC3)c21 ; Cn1nc(C2CCC(=O)NC2=O)c2cccc(OC3CCN(C(=O)C#Cc4ccccc4)CC3)c21]

3-(7-((1-(2,4-Dimethylthiazole-5-carbonyl)piperidin-4-yl)oxy)-1-methyl-1H-indazol-3-yl)piperidine-2,6-dione C033. MS (ESI) m/z: 482.2 [M+1]⁺.

3-(1-Methyl-7-((1-(2-(p-tolylthio) acetyl)piperidin-4-yl)oxy)-1H-indazol-3-yl)-piperidine-2,6-dione C034. MS (ESI) m/z: 507.2 [M+1]⁺.

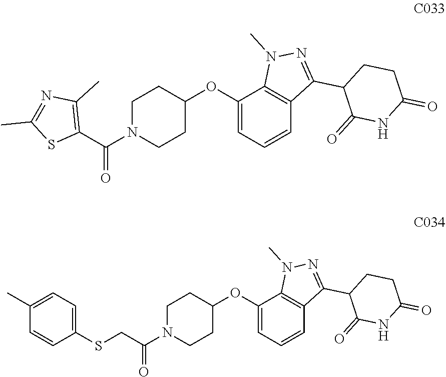 [SMILES: Cc1nc(C)c(C(=O)N2CCC(Oc3cccc4c(C5CCC(=O)NC5=O)nn(C)c34)CC2)s1 ; Cc1ccc(SCC(=O)N2CCC(Oc3cccc4c(C5CCC(=O)NC5=O)nn(C)c34)CC2)cc1]

2-(4-((3-(2,6-Dioxopiperidin-3-yl)-1-methyl-1H-indazol-7-yl)oxy) piperidin-1-yl)-N-(2-methoxyphenyl)-2-oxoacetamide C035. MS (ESI) m/z: 520.2 [M+1]⁺.

3-(7-((1-(1,5-Dimethyl-1H-pyrazole-4-carbonyl)piperidin-4-yl)oxy)-1-methyl-1H-indazol-3-yl)piperidine-2,6-dione C036. MS (ESI) m/z: 465.2 [M+1]⁺.

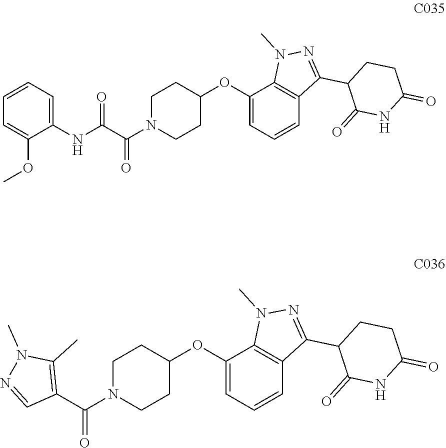 [SMILES: COc1ccccc1NC(=O)C(=O)N1CCC(Oc2cccc3c(C4CCC(=O)NC4=O)nn(C)c23)CC1 ; Cc1c(C(=O)N2CCC(Oc3cccc4c(C5CCC(=O)NC5=O)nn(C)c34)CC2)cnn1C]

3-(7-((1-(1-Hydroxycyclopropane-1-carbonyl)piperidin-4-yl)oxy)-1-methyl-1H-indazol-3-yl)piperidine-2,6-dione C037. MS (ESI) m/z: 427.2 [M+1]⁺.

3-(7-((1-(3-(Difluoromethyl)-5-fluoro-1-methyl-1H-pyrazole-4-carbonyl)-piperidin-4-yl)oxy)-1-methyl-1H-indazol-3-yl)piperidine-2,6-dione C038. MS (ESI) m/z: 519.2 [M+1]⁺.

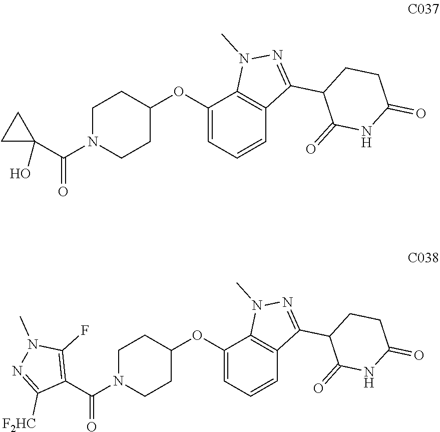 [SMILES: Cn1nc(C2CCC(=O)NC2=O)c2cccc(OC3CCN(C(=O)C4(O)CC4)CC3)c21 ; Cn1nc(C(F)F)c(C(=O)N2CCC(Oc3cccc4c(C5CCC(=O)NC5=O)nn(C)c34)CC2)c1F]

N-(2-(4-((3-(2,6-Dioxopiperidin-3-yl)-1-methyl-1H-indazol-7-yl)oxy) piperidin-1-yl)-2-oxoethyl)-2-phenylacetamide C039. MS (ESI) m/z: 518.3 [M+1]⁺.

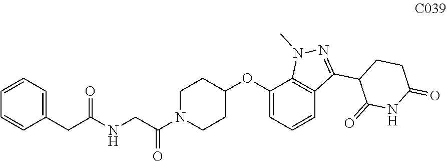 [SMILES: Cn1nc(C2CCC(=O)NC2=O)c2cccc(OC3CCN(C(=O)CNC(=O)Cc4ccccc4)CC3)c21]

3-(1-Methyl-7-((1-(4,5,6,7-tetrahydro-1H-indazole-3-carbonyl)piperidin-4-yl)-oxy)-1H-indazol-3-yl)piperidine-2,6-dione C040. MS (ESI) m/z: 491.3 [M+1]⁺.

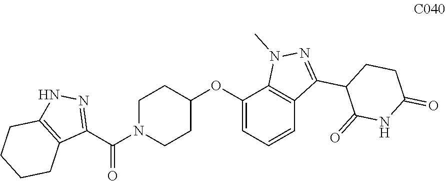 [SMILES: Cn1nc(C2CCC(=O)NC2=O)c2cccc(OC3CCN(C(=O)c4n[nH]c5c4CCCC5)CC3)c21]

3-(1-Methyl-7-((1-(6-methylbenzo[b]thiophene-2-carbonyl)piperidin-4-yl)oxy)-1H-indazol-3-yl)piperidine-2,6-dione C041. MS (ESI) m/z: 517.2 [M+1]⁺.

3-(7-((1-(1-(Hydroxymethyl)cyclopropane-1-carbonyl)piperidin-4-yl)oxy)-1-methyl-1H-indazol-3-yl)piperidine-2,6-dione C042. MS (ESI) m/z: 441.2 [M+1]⁺.

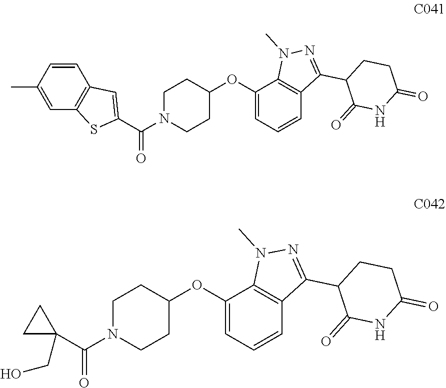 [SMILES: Cc1ccc2cc(C(=O)N3CCC(Oc4cccc5c(C6CCC(=O)NC6=O)nn(C)c45)CC3)sc2c1 ; Cn1nc(C2CCC(=O)NC2=O)c2cccc(OC3CCN(C(=O)C4(CO)CC4)CC3)c21]

3-(1-Methyl-7-((1-(3-phenoxybenzoyl)piperidin-4-yl)oxy)-1H-indazol-3-yl)-piperidine-2,6-dione C043. MS (ESI) m/z: 539.2 [M+1]⁺.

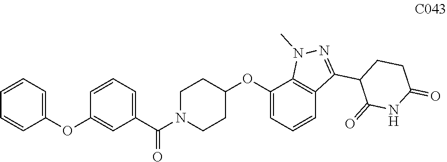 [SMILES: Cn1nc(C2CCC(=O)NC2=O)c2cccc(OC3CCN(C(=O)c4cccc(Oc5ccccc5)c4)CC3)c21]

3-(7-((1-(3-Hydroxy-4-methoxybenzoyl)piperidin-4-yl)oxy)-1-methyl-1H-indazol-3-yl)piperidine-2,6-dione C044. MS (ESI) m/z: 493.2 [M+1]⁺.

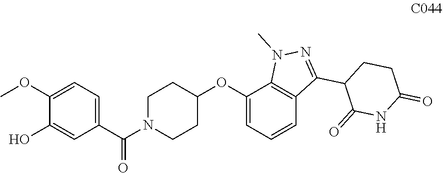 [SMILES: COc1ccc(C(=O)N2CCC(Oc3cccc4c(C5CCC(=O)NC5=O)nn(C)c34)CC2)cc1O]

3-(7-((1-(4′-Fluoro-[1,1′-biphenyl]-3-carbonyl)piperidin-4-yl)oxy)-1-methyl-1H-indazol-3-yl)piperidine-2,6-dione C045. MS (ESI) m/z: 541.2 [M+1]⁺.

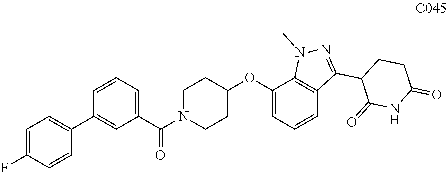 [SMILES: Cn1nc(C2CCC(=O)NC2=O)c2cccc(OC3CCN(C(=O)c4cccc(-c5ccc(F)cc5)c4)CC3)c21]

3-(7-((1-(5-Amino-1-phenyl-1H-pyrazole-4-carbonyl)piperidin-4-yl)oxy)-1-methyl-1H-indazol-3-yl)piperidine-2,6-dione C046. MS (ESI) m/z: 528.2 [M+1]⁺.

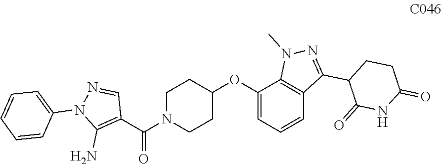 [SMILES: Cn1nc(C2CCC(=O)NC2=O)c2cccc(OC3CCN(C(=O)c4cnn(-c5ccccc5)c4N)CC3)c21]

3-(7-((1-(6,7-Dihydro-4H-pyrazolo[5,1-c][1,4]oxazine-2-carbonyl)piperidin-4-yl)oxy)-1-methyl-1H-indazol-3-yl)piperidine-2,6-dione C047. MS (ESI) m/z: 493.2 [M+1]⁺.

4-Amino-5-(4-((3-(2,6-dioxopiperidin-3-yl)-1-methyl-1H-indazol-7-yl)oxy)-piperidine-1-carbonyl) isothiazole-3-carboxamide C048. MS (ESI) m/z: 512.2 [M+1]⁺.

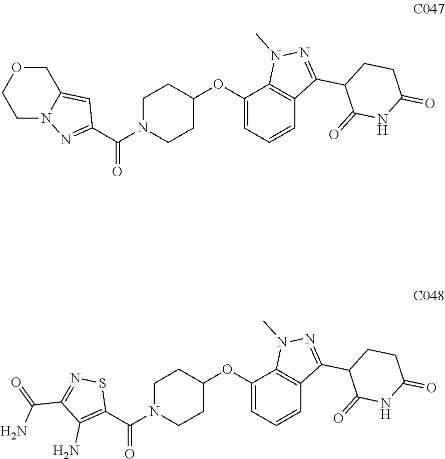 [SMILES: Cn1nc(C2CCC(=O)NC2=O)c2cccc(OC3CCN(C(=O)c4cc5n(n4)CCOC5)CC3)c21 ; Cn1nc(C2CCC(=O)NC2=O)c2cccc(OC3CCN(C(=O)c4snc(C(N)=O)c4N)CC3)c21]

3-(1-Methyl-7-((1-(5-(pyridin-3-yl)-1H-imidazole-4-carbonyl)piperidin-4-yl)-oxy)-1H-indazol-3-yl)piperidine-2,6-dione C049. MS (ESI) m/z: 514.2 [M+1]⁺.

3-(1-Methyl-7-((1-(1-methyl-4-oxo-1,4-dihydropyrrolo[1,2-b]pyridazine-3-carbonyl)piperidin-4-yl)oxy)-1H-indazol-3-yl)piperidine-2,6-dione C050. MS (ESI) m/z: 517.2 [M+1]⁺.

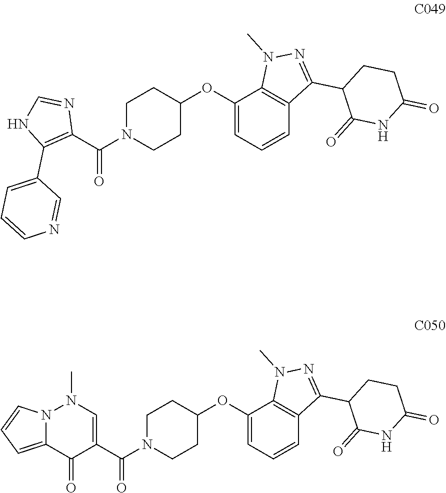 [SMILES: Cn1nc(C2CCC(=O)NC2=O)c2cccc(OC3CCN(C(=O)c4nc[nH]c4-c4cccnc4)CC3)c21 ; Cn1nc(C2CCC(=O)NC2=O)c2cccc(OC3CCN(C(=O)c4cn(C)n5cccc5c4=O)CC3)c21]

3-(7-((1-(6-Ethoxypicolinoyl)piperidin-4-yl)oxy)-1-methyl-1H-indazol-3-yl)-piperidine-2,6-dione C051. MS (ESI) m/z: 492.2 [M+1]⁺.

3-(1-Methyl-7-((1-(2,4,5,6-tetrahydrocyclopenta[c]pyrrole-1-carbonyl)piperidin-4-yl)oxy)-1H-indazol-3-yl)piperidine-2,6-dione C052. MS (ESI) m/z: 476.2 [M+1]⁺.

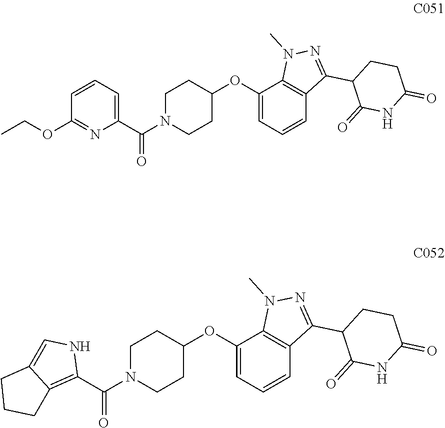 [SMILES: CCOc1cccc(C(=O)N2CCC(Oc3cccc4c(C5CCC(=O)NC5=O)nn(C)c34)CC2)n1 ; Cn1nc(C2CCC(=O)NC2=O)c2cccc(OC3CCN(C(=O)c4[nH]cc5c4CCC5)CC3)c21]

3-Amino-5-(4-((3-(2,6-dioxopiperidin-3-yl)-1-methyl-1H-indazol-7-yl)oxy)-piperidine-1-carbonyl)benzonitrile C053. MS (ESI) m/z: 487.2 [M+1]⁺.

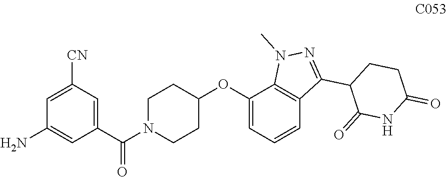 [SMILES: Cn1nc(C2CCC(=O)NC2=O)c2cccc(OC3CCN(C(=O)c4cc(N)cc(C#N)c4)CC3)c21]

3-(7-((1-(2-(Benzyloxy)-2-methylpropanoyl)piperidin-4-yl)oxy)-1-methyl-1H-indazol-3-yl)piperidine-2,6-dione C054. MS (ESI) m/z: 519.3 [M+1]⁺.

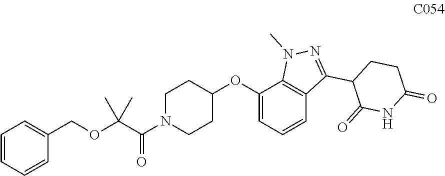 [SMILES: Cn1nc(C2CCC(=O)NC2=O)c2cccc(OC3CCN(C(=O)C(C)(C)OCc4ccccc4)CC3)c21]

3-(7-((1-(1H-Pyrazole-4-carbonyl)piperidin-4-yl)oxy)-1-methyl-1H-indazol-3-yl)-piperidine-2,6-dione C055. MS (ESI) m/z: 437.2 [M+1]⁺.

3-(7-((1-(²H-Indazole-5-carbonyl)piperidin-4-yl)oxy)-1-methyl-1H-indazol-3-yl)-piperidine-2,6-dione C056. MS (ESI) m/z: 487.2 [M+1]⁺.

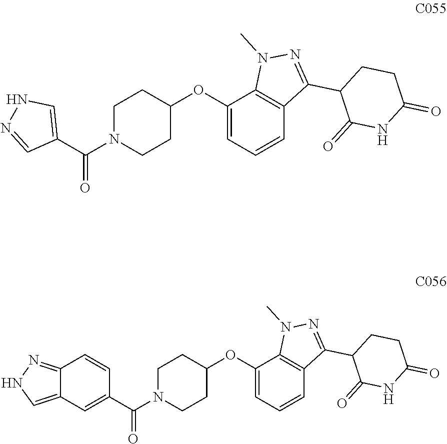 [SMILES: Cn1nc(C2CCC(=O)NC2=O)c2cccc(OC3CCN(C(=O)c4cn[nH]c4)CC3)c21 ; Cn1nc(C2CCC(=O)NC2=O)c2cccc(OC3CCN(C(=O)c4ccc5n[nH]cc5c4)CC3)c21]

3-(7-((1-(1-(Tert-butyl)-1H-1,2,3-triazole-4-carbonyl)piperidin-4-yl)oxy)-1-methyl-1H-indazol-3-yl)piperidine-2,6-dione C057. MS (ESI) m/z: 494.3 [M+1]⁺.

3-(1-Methyl-7-((1-(5-(pyridin-3-yl)-1H-pyrazole-3-carbonyl)piperidin-4-yl)oxy)-1H-indazol-3-yl)piperidine-2,6-dione C058. MS (ESI) m/z: 514.2 [M+1]⁺.

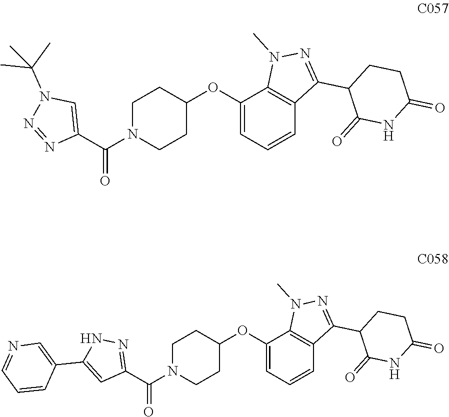 [SMILES: Cn1nc(C2CCC(=O)NC2=O)c2cccc(OC3CCN(C(=O)c4cn(C(C)(C)C)nn4)CC3)c21 ; Cn1nc(C2CCC(=O)NC2=O)c2cccc(OC3CCN(C(=O)c4cc(-c5cccnc5)[nH]n4)CC3)c21]

3-(7-((1-(2-Mercapto-1-methyl-1H-imidazole-5-carbonyl)piperidin-4-yl)oxy)-1-methyl-1H-indazol-3-yl)piperidine-2,6-dione C059. MS (ESI) m/z: 483.1 [M+1]⁺.

3-(7-((1-(3-(Benzo[d][1,3]dioxol-5-yl) acryloyl)piperidin-4-yl)oxy)-1-methyl-1H-indazol-3-yl)piperidine-2,6-dione C060. MS (ESI) m/z: 517.2 [M+1]⁺.

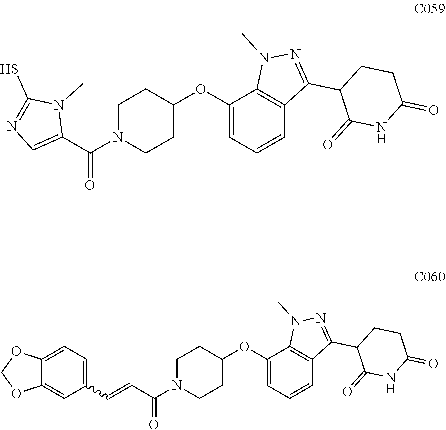 [SMILES: Cn1c(C(=O)N2CCC(Oc3cccc4c(C5CCC(=O)NC5=O)nn(C)c34)CC2)cnc1S ; Cn1nc(C2CCC(=O)NC2=O)c2cccc(OC3CCN(C(=O)C=Cc4ccc5c(c4)OCO5)CC3)c21]

3-(7-((1-(4-Amino-1,2,5-oxadiazole-3-carbonyl)piperidin-4-yl)oxy)-1-methyl-1H-indazol-3-yl)piperidine-2,6-dione C061. MS (ESI) m/z: 454.2 [M+1]⁺.

3-(7-((1-(2-Aminothiazole-4-carbonyl)piperidin-4-yl)oxy)-1-methyl-1H-indazol-3-yl)piperidine-2,6-dione C062. MS (ESI) m/z: 469.1 [M+1]⁺.

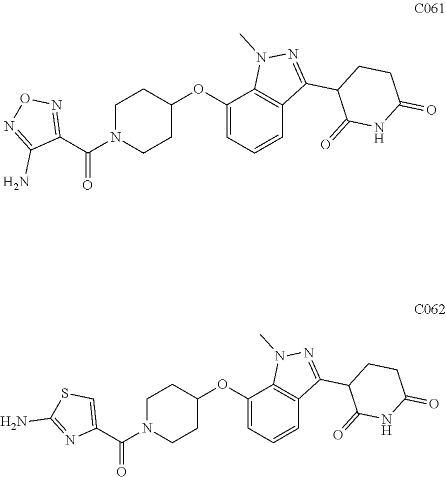 [SMILES: Cn1nc(C2CCC(=O)NC2=O)c2cccc(OC3CCN(C(=O)c4nonc4N)CC3)c21 ; Cn1nc(C2CCC(=O)NC2=O)c2cccc(OC3CCN(C(=O)c4csc(N)n4)CC3)c21]

3-(7-((1-(4-Benzyl-5-oxomorpholine-3-carbonyl)piperidin-4-yl)oxy)-1-methyl-1H-indazol-3-yl)piperidine-2,6-dione C063. MS (ESI) m/z: 560.3 [M+1]⁺.

3-(7-((1-(Imidazo[2,1-b]thiazole-5-carbonyl)piperidin-4-yl)oxy)-1-methyl-1H-indazol-3-yl)piperidine-2,6-dione C064. MS (ESI) m/z: 493.1 [M+1]⁺.

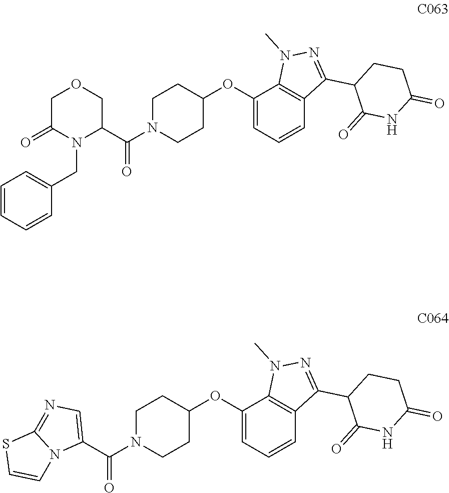 [SMILES: Cn1nc(C2CCC(=O)NC2=O)c2cccc(OC3CCN(C(=O)C4COCC(=O)N4Cc4ccccc4)CC3)c21 ; Cn1nc(C2CCC(=O)NC2=O)c2cccc(OC3CCN(C(=O)c4cnc5sccn45)CC3)c21]

3-(1-Methyl-7-((1-(3-(phenylsulfonyl) propanoyl)piperidin-4-yl)oxy)-1H-indazol-3-yl)piperidine-2,6-dione C065. MS (ESI) m/z: 539.2 [M+1]⁺.

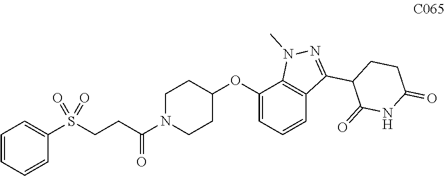 [SMILES: Cn1nc(C2CCC(=O)NC2=O)c2cccc(OC3CCN(C(=O)CCS(=O)(=O)c4ccccc4)CC3)c21]

5-(4-((3-(2,6-Dioxopiperidin-3-yl)-1-methyl-1H-indazol-7-yl)oxy) piperidine-1-carbonyl) pyrazine-2-carbonitrile C066. MS (ESI) m/z: 474.2 [M+1]⁺.

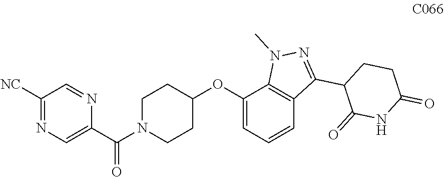 [SMILES: Cn1nc(C2CCC(=O)NC2=O)c2cccc(OC3CCN(C(=O)c4cnc(C#N)cn4)CC3)c21]

3-(7-((1-(4-Hydroxycyclohexane-1-carbonyl)piperidin-4-yl)oxy)-1-methyl-1H-indazol-3-yl)piperidine-2,6-dione C067. MS (ESI) m/z: 469.2 [M+1]⁺.

3-(1-Methyl-7-((1-(4,5,6,7-tetrahydrobenzo[b]thiophene-2-carbonyl)piperidin-4-yl)oxy)-1H-indazol-3-yl)piperidine-2,6-dione C068. MS (ESI) m/z: 507.2 [M+1]⁺.

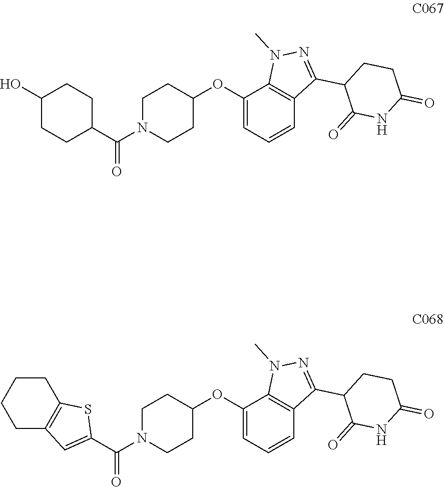 [SMILES: Cn1nc(C2CCC(=O)NC2=O)c2cccc(OC3CCN(C(=O)C4CCC(O)CC4)CC3)c21 ; Cn1nc(C2CCC(=O)NC2=O)c2cccc(OC3CCN(C(=O)c4cc5c(s4)CCCC5)CC3)c21]

3-(7-((1-(4,6-Dihydroxypyridazine-3-carbonyl)piperidin-4-yl)oxy)-1-methyl-1H-indazol-3-yl)piperidine-2,6-dione C069. MS (ESI) m/z: 481.1 [M+1]⁺.

3-(7-((1-(2,3-Dihydrobenzofuran-7-carbonyl)piperidin-4-yl)oxy)-1-methyl-1H-indazol-3-yl)piperidine-2,6-dione C070. MS (ESI) m/z: 489.2 [M+1]⁺.

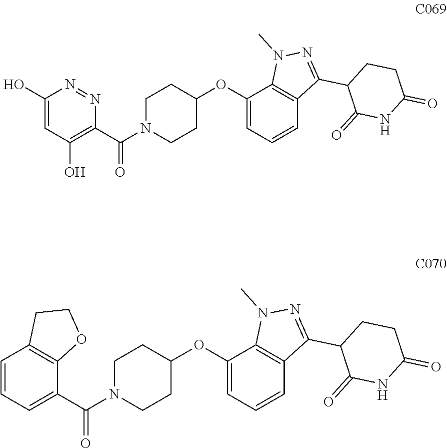 [SMILES: Cn1nc(C2CCC(=O)NC2=O)c2cccc(OC3CCN(C(=O)c4nnc(O)cc4O)CC3)c21 ; Cn1nc(C2CCC(=O)NC2=O)c2cccc(OC3CCN(C(=O)c4cccc5c4OCC5)CC3)c21]

3-(7-((1-(6-Methoxy-1H-indazole-3-carbonyl)piperidin-4-yl)oxy)-1-methyl-1H-indazol-3-yl)piperidine-2,6-dione C071. MS (ESI) m/z: 517.2 [M+1]⁺.

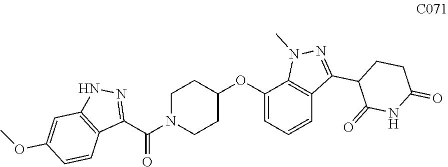 [SMILES: COc1ccc2c(C(=O)N3CCC(Oc4cccc5c(C6CCC(=O)NC6=O)nn(C)c45)CC3)n[nH]c2c1]

(E)-3-(7-((1-(3-(3,4-Difluorophenyl) acryloyl)piperidin-4-yl)oxy)-1-methyl-1H-indazol-3-yl)piperidine-2,6-dione C072. MS (ESI) m/z: 509.2 [M+1]⁺.

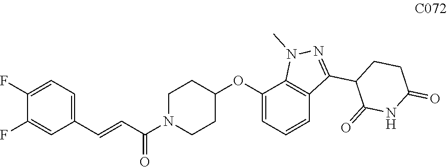 [SMILES: Cn1nc(C2CCC(=O)NC2=O)c2cccc(OC3CCN(C(=O)/C=C/c4ccc(F)c(F)c4)CC3)c21]

3-(1-Methyl-7-((1-(3-phenylcyclobutane-1-carbonyl)piperidin-4-yl)oxy)-1H-indazol-3-yl)piperidine-2,6-dione C073. MS (ESI) m/z: 501.3 [M+1]⁺.

3-(1-Methyl-7-((1-(2-methyl-1H-imidazole-5-carbonyl)piperidin-4-yl)oxy)-1H-indazol-3-yl)piperidine-2,6-dione C074. MS (ESI) m/z: 451.2 [M+1]⁺.

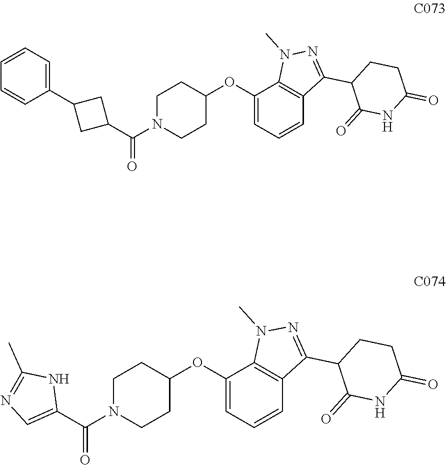 [SMILES: Cn1nc(C2CCC(=O)NC2=O)c2cccc(OC3CCN(C(=O)C4CC(c5ccccc5)C4)CC3)c21 ; Cc1ncc(C(=O)N2CCC(Oc3cccc4c(C5CCC(=O)NC5=O)nn(C)c34)CC2)[nH]1]

3-(7-((1-(2-(Cyclopropylamino) thiazole-5-carbonyl)piperidin-4-yl)oxy)-1-methyl-1H-indazol-3-yl)piperidine-2,6-dione C075. MS (ESI) m/z: 509.2 [M+1]⁺.

5-(4-((3-(2,6-Dioxopiperidin-3-yl)-1-methyl-1H-indazol-7-yl)oxy) piperidine-1-carbonyl) furan-3-carbonitrile C076. MS (ESI) m/z: 462.2 [M+1]⁺.

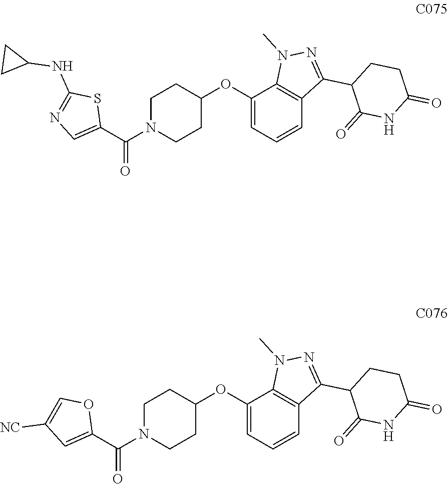 [SMILES: Cn1nc(C2CCC(=O)NC2=O)c2cccc(OC3CCN(C(=O)c4cnc(NC5CC5)s4)CC3)c21 ; Cn1nc(C2CCC(=O)NC2=O)c2cccc(OC3CCN(C(=O)c4cc(C#N)co4)CC3)c21]

3-(7-((1-(2-Cyclopropylacetyl)piperidin-4-yl)oxy)-1-methyl-1H-indazol-3-yl)-piperidine-2,6-dione C077. MS (ESI) m/z: 425.2 [M+1]⁺.

4-(4-((3-(2,6-Dioxopiperidin-3-yl)-1-methyl-1H-indazol-7-yl)oxy) piperidine-1-carbonyl)piperidine-1-carboximidamide C078. MS (ESI) m/z: 496.3 [M+1]⁺.

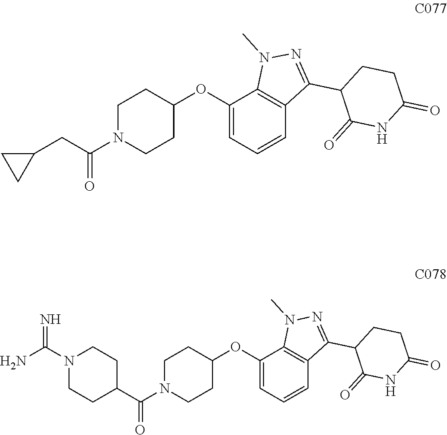 [SMILES: Cn1nc(C2CCC(=O)NC2=O)c2cccc(OC3CCN(C(=O)CC4CC4)CC3)c21 ; Cn1nc(C2CCC(=O)NC2=O)c2cccc(OC3CCN(C(=O)C4CCN(C(=N)N)CC4)CC3)c21]

N-Cyclohexyl-2-(4-((3-(2,6-dioxopiperidin-3-yl)-1-methyl-1H-indazol-7-yl)oxy)-piperidin-1-yl)-2-oxoacetamide C079. MS (ESI) m/z: 496.2 [M+1]⁺.

5-(4-((3-(2,6-Dioxopiperidin-3-yl)-1-methyl-1H-indazol-7-yl)oxy) piperidine-1-carbonyl)-1H-pyrrole-3-carbonitrile C080. MS (ESI) m/z: 461.2 [M+1]⁺.

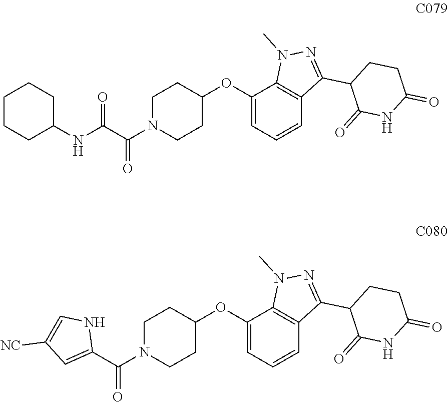 [SMILES: Cn1nc(C2CCC(=O)NC2=O)c2cccc(OC3CCN(C(=O)C(=O)NC4CCCCC4)CC3)c21 ; Cn1nc(C2CCC(=O)NC2=O)c2cccc(OC3CCN(C(=O)c4cc(C#N)c[nH]4)CC3)c21]

3-(7-((1-(4-(1H-Imidazol-2-yl)benzoyl)piperidin-4-yl)oxy)-1-methyl-1H-indazol-3-yl)piperidine-2,6-dione C081. MS (ESI) m/z: 513.2 [M+1]⁺.

3-(7-((1-(5-Amino-2-methylthiazole-4-carbonyl)piperidin-4-yl)oxy)-1-methyl-1H-indazol-3-yl)piperidine-2,6-dione C082. MS (ESI) m/z: 483.2 [M+1]⁺.

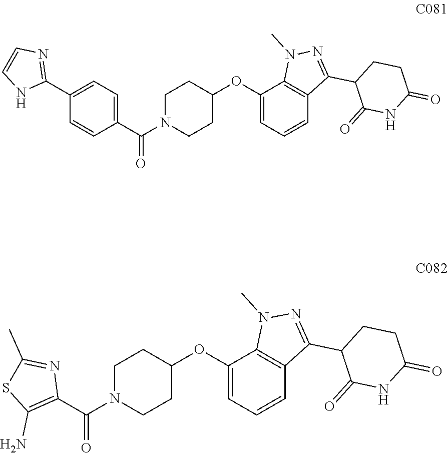 [SMILES: Cn1nc(C2CCC(=O)NC2=O)c2cccc(OC3CCN(C(=O)c4ccc(-c5ncc[nH]5)cc4)CC3)c21 ; Cc1nc(C(=O)N2CCC(Oc3cccc4c(C5CCC(=O)NC5=O)nn(C)c34)CC2)c(N)s1]

N-(4-(4-((3-(2,6-Dioxopiperidin-3-yl)-1-methyl-1H-indazol-7-yl)oxy) piperidine-1-carbonyl)phenyl)benzenesulfonamide C083. MS (ESI) m/z: 602.2 [M+1]⁺.

3-(7-((1-(Cyclobut-1-ene-1-carbonyl)piperidin-4-yl)oxy)-1-methyl-1H-indazol-3-yl)piperidine-2,6-dione C084. MS (ESI) m/z: 423.2 [M+1]⁺.

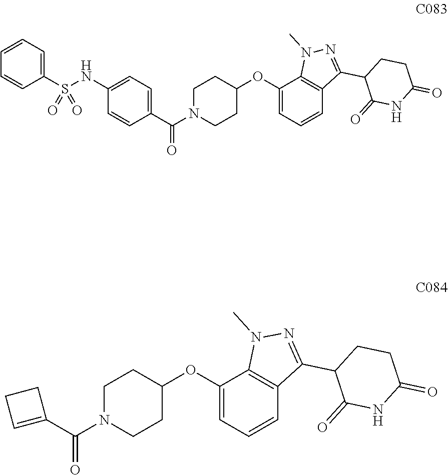 [SMILES: Cn1nc(C2CCC(=O)NC2=O)c2cccc(OC3CCN(C(=O)c4ccc(NS(=O)(=O)c5ccccc5)cc4)CC3)c21 ; Cn1nc(C2CCC(=O)NC2=O)c2cccc(OC3CCN(C(=O)C4=CCC4)CC3)c21]

3-(1-Methyl-7-((1-(4-oxo-4,5,6,7-tetrahydropyrazolo[1,5-a]pyrazine-2-carbonyl)-piperidin-4-yl)oxy)-1H-indazol-3-yl)piperidine-2,6-dione C085. MS (ESI) m/z: 506.2 [M+1]⁺.

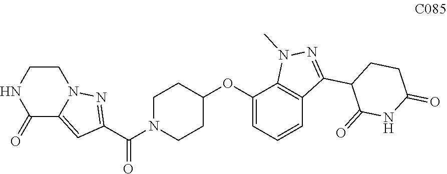 [SMILES: Cn1nc(C2CCC(=O)NC2=O)c2cccc(OC3CCN(C(=O)c4cc5n(n4)CCNC5=O)CC3)c21]

3-(7-((1-(3-(3-Fluorophenyl) propioloyl)piperidin-4-yl)oxy)-1-methyl-1H-indazol-3-yl)piperidine-2,6-dione C086. MS (ESI) m/z: 489.2 [M+1]⁺.

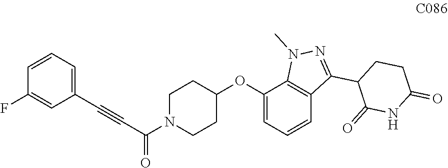 [SMILES: Cn1nc(C2CCC(=O)NC2=O)c2cccc(OC3CCN(C(=O)C#Cc4cccc(F)c4)CC3)c21]

3-(7-((1-(4H-Thieno[3,2-b]pyrrole-5-carbonyl)piperidin-4-yl)oxy)-1-methyl-1H-indazol-3-yl)piperidine-2,6-dione C087. MS (ESI) m/z: 492.2 [M+1]⁺.

3-(1-Methyl-7-((1-(2-methyl-4-(trifluoromethyl) furan-3-carbonyl)piperidin-4-yl)-oxy)-1H-indazol-3-yl)piperidine-2,6-dione C088. MS (ESI) m/z: 519.2 [M+1]⁺.

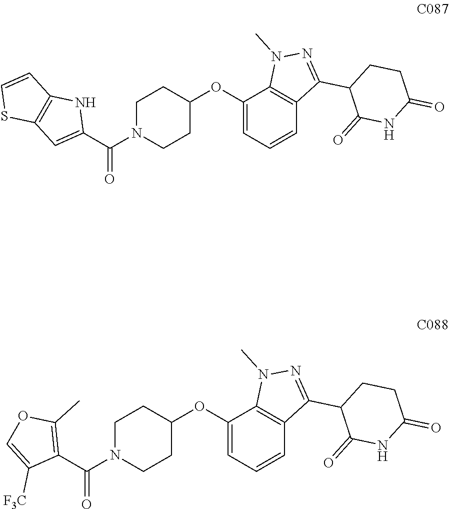 [SMILES: Cn1nc(C2CCC(=O)NC2=O)c2cccc(OC3CCN(C(=O)c4cc5sccc5[nH]4)CC3)c21 ; Cc1occ(C(F)(F)F)c1C(=O)N1CCC(Oc2cccc3c(C4CCC(=O)NC4=O)nn(C)c23)CC1]

3-(7-((1-(2,5-Dimethyloxazole-4-carbonyl)piperidin-4-yl)oxy)-1-methyl-1H-indazol-3-yl)piperidine-2,6-dione C089. MS (ESI) m/z: 466.2 [M+1]⁺.

3-(7-((1-(2-(2-Mercapto-4-methylthiazol-5-yl)acetyl)piperidin-4-yl)oxy)-1-methyl-1H-indazol-3-yl)piperidine-2,6-dione C090. MS (ESI) m/z: 514.2 [M+1]⁺.

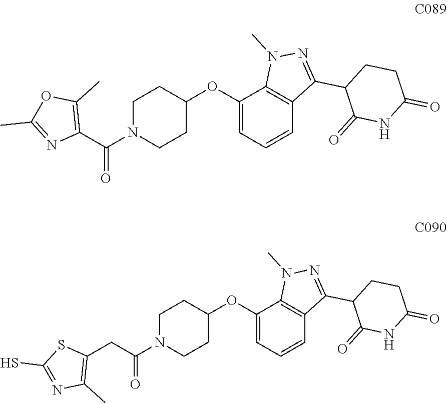 [SMILES: Cc1nc(C(=O)N2CCC(Oc3cccc4c(C5CCC(=O)NC5=O)nn(C)c34)CC2)c(C)o1 ; Cc1nc(S)sc1CC(=O)N1CCC(Oc2cccc3c(C4CCC(=O)NC4=O)nn(C)c23)CC1]

3-(7-((1-(4-Hydroxy-3-(trifluoromethyl)benzoyl)piperidin-4-yl)oxy)-1-methyl-1H-indazol-3-yl)piperidine-2,6-dione C091. MS (ESI) m/z: 531.2 [M+1]⁺.

3-(7-((1-(1H-Pyrrolo[2,3-b]pyridine-3-carbonyl)piperidin-4-yl)oxy)-1-methyl-1H-indazol-3-yl)piperidine-2,6-dione C092. MS (ESI) m/z: 487.2 [M+1]⁺.

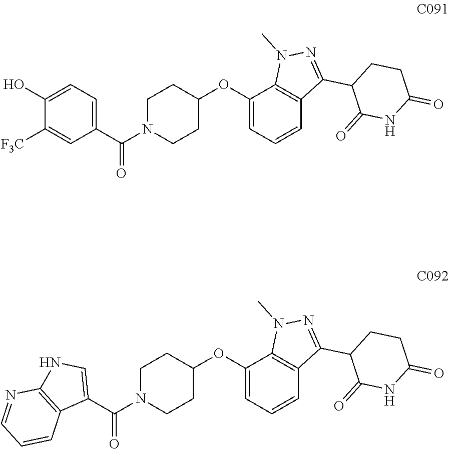 [SMILES: Cn1nc(C2CCC(=O)NC2=O)c2cccc(OC3CCN(C(=O)c4ccc(O)c(C(F)(F)F)c4)CC3)c21 ; Cn1nc(C2CCC(=O)NC2=O)c2cccc(OC3CCN(C(=O)c4c[nH]c5ncccc45)CC3)c21]

4-(4-((3-(2,6-Dioxopiperidin-3-yl)-1-methyl-1H-indazol-7-yl)oxy) piperidine-1-carbonyl)tetrahydro-2H-pyran-4-carbonitrile C093. MS (ESI) m/z: 480.3 [M+1]⁺.

3-(7-((1-(3-Hydroxypropanoyl)piperidin-4-yl)oxy)-1-methyl-1H-indazol-3-yl)-piperidine-2,6-dione C094. MS (ESI) m/z: 415.2 [M+1]⁺.

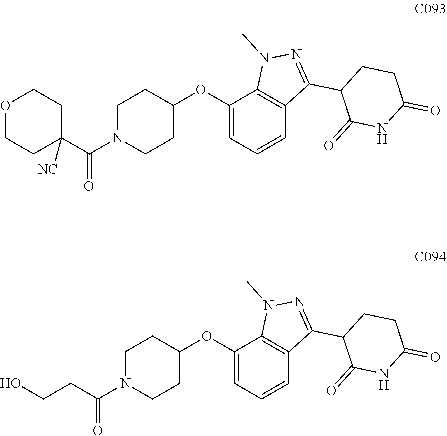 [SMILES: Cn1nc(C2CCC(=O)NC2=O)c2cccc(OC3CCN(C(=O)C4(C#N)CCOCC4)CC3)c21 ; Cn1nc(C2CCC(=O)NC2=O)c2cccc(OC3CCN(C(=O)CCO)CC3)c21]

3-(1-Methyl-7-((1-(pent-2-ynoyl)piperidin-4-yl)oxy)-1H-indazol-3-yl)piperidine-2,6-dione C095. MS (ESI) m/z: 423.2 [M+1]⁺.

3-(7-((1-(2-Isopropylisonicotinoyl)piperidin-4-yl)oxy)-1-methyl-1H-indazol-3-yl)piperidine-2,6-dione C096. MS (ESI) m/z: 490.2 [M+1]⁺.

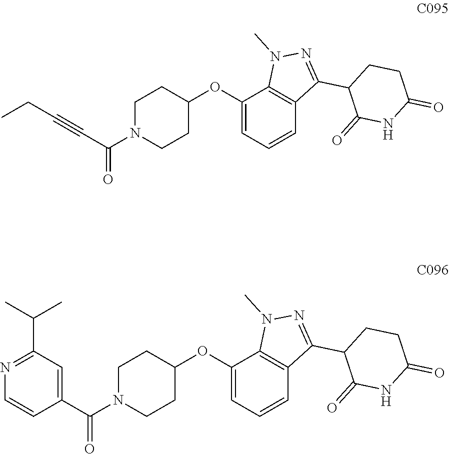 [SMILES: CCC#CC(=O)N1CCC(Oc2cccc3c(C4CCC(=O)NC4=O)nn(C)c23)CC1 ; CC(C)c1cc(C(=O)N2CCC(Oc3cccc4c(C5CCC(=O)NC5=O)nn(C)c34)CC2)ccn1]

2-(2-(4-((3-(2,6-Dioxopiperidin-3-yl)-1-methyl-1H-indazol-7-yl)oxy) piperidin-1-yl)-2-oxoethoxy)benzonitrile C097. MS (ESI) m/z: 502.2 [M+1]⁺.

3-(7-((1-(6,7-Dihydro-5H-pyrazolo[5,1-b][1,3]oxazine-2-carbonyl)piperidin-4-yl)oxy)-1-methyl-1H-indazol-3-yl)piperidine-2,6-dione C098. MS (ESI) m/z: 493.2 [M+1]⁺.

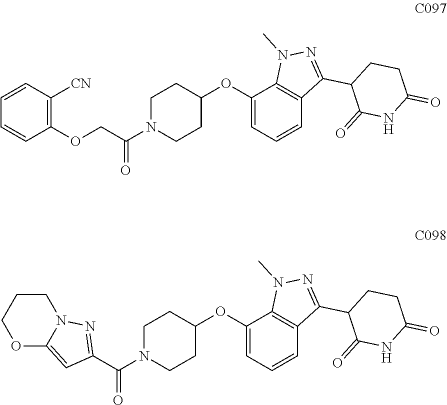 [SMILES: Cn1nc(C2CCC(=O)NC2=O)c2cccc(OC3CCN(C(=O)COc4ccccc4C#N)CC3)c21 ; Cn1nc(C2CCC(=O)NC2=O)c2cccc(OC3CCN(C(=O)c4cc5n(n4)CCCO5)CC3)c21]

3-(1-Methyl-7-((1-(4-oxo-4H-chromene-3-carbonyl)piperidin-4-yl)oxy)-1H-indazol-3-yl)piperidine-2,6-dione C099. MS (ESI) m/z: 515.2 [M+1]⁺.

3-(1-Methyl-7-((1-(3-methylisothiazole-5-carbonyl)piperidin-4-yl)oxy)-1H-indazol-3-yl)piperidine-2,6-dione C100. MS (ESI) m/z: 468.2 [M+1]⁺.

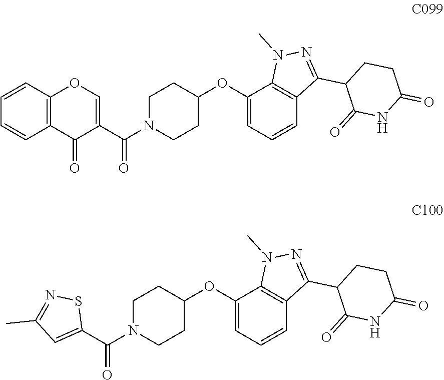 [SMILES: Cn1nc(C2CCC(=O)NC2=O)c2cccc(OC3CCN(C(=O)c4coc5ccccc5c4=O)CC3)c21 ; Cc1cc(C(=O)N2CCC(Oc3cccc4c(C5CCC(=O)NC5=O)nn(C)c34)CC2)sn1]

3-(7-((1-(2-((4-Chlorophenyl)thio) acetyl)piperidin-4-yl)oxy)-1-methyl-1H-indazol-3-yl)piperidine-2,6-dione C101. MS (ESI) m/z: 527.2 [M+1]⁺.

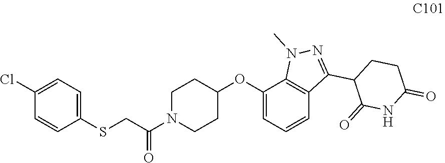 [SMILES: Cn1nc(C2CCC(=O)NC2=O)c2cccc(OC3CCN(C(=O)CSc4ccc(Cl)cc4)CC3)c21]

3-(7-((1-(Imidazo[1,2-a]pyridine-3-carbonyl)piperidin-4-yl)oxy)-1-methyl-1H-indazol-3-yl)piperidine-2,6-dione C102. MS (ESI) m/z: 487.2 [M+1]⁺.

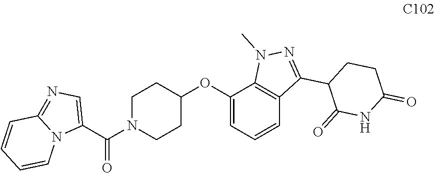 [SMILES: Cn1nc(C2CCC(=O)NC2=O)c2cccc(OC3CCN(C(=O)c4cnc5ccccn45)CC3)c21]

3-(1-Methyl-7-((1-(5-methylthiophene-2-carbonyl)piperidin-4-yl)oxy)-1H-indazol-3-yl)piperidine-2,6-dione C103. MS (ESI) m/z: 467.2 [M+1]⁺.

3-(7-((1-(5-Isopropyl-1H-pyrazole-3-carbonyl)piperidin-4-yl)oxy)-1-methyl-1H-indazol-3-yl)piperidine-2,6-dione C104. MS (ESI) m/z: 479.3 [M+1]⁺.

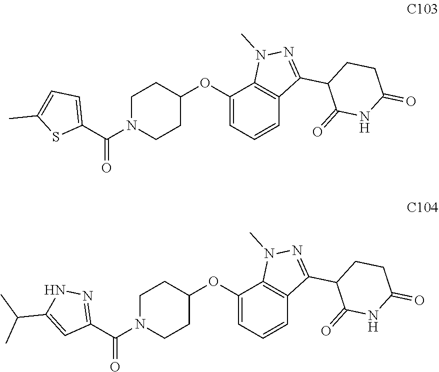 [SMILES: Cc1ccc(C(=O)N2CCC(Oc3cccc4c(C5CCC(=O)NC5=O)nn(C)c34)CC2)s1 ; CC(C)c1cc(C(=O)N2CCC(Oc3cccc4c(C5CCC(=O)NC5=O)nn(C)c34)CC2)n[nH]1]

3-(7-((1-(4-(Tert-butyl)benzoyl)piperidin-4-yl)oxy)-1-methyl-1H-indazol-3-yl)-piperidine-2,6-dione C105. MS (ESI) m/z: 503.3 [M+1]⁺.

3-(1-Methyl-7-((1-(4,5,6,7-tetrahydrobenzo[b]thiophene-3-carbonyl)piperidin-4-yl)oxy)-1H-indazol-3-yl)piperidine-2,6-dione C106. MS (ESI) m/z: 507.2 [M+1]⁺.

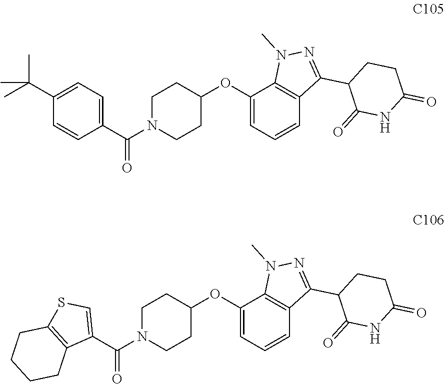 [SMILES: Cn1nc(C2CCC(=O)NC2=O)c2cccc(OC3CCN(C(=O)c4ccc(C(C)(C)C)cc4)CC3)c21 ; Cn1nc(C2CCC(=O)NC2=O)c2cccc(OC3CCN(C(=O)c4csc5c4CCCC5)CC3)c21]

3-(1-Methyl-7-((1-(thiazole-5-carbonyl)piperidin-4-yl)oxy)-1H-indazol-3-yl)-piperidine-2,6-dione C107. MS (ESI) m/z: 454.1 [M+1]⁺.

3-(1-Methyl-7-((1-(oxazole-4-carbonyl)piperidin-4-yl)oxy)-1H-indazol-3-yl)-piperidine-2,6-dione C108. MS (ESI) m/z: 438.2 [M+1]⁺.

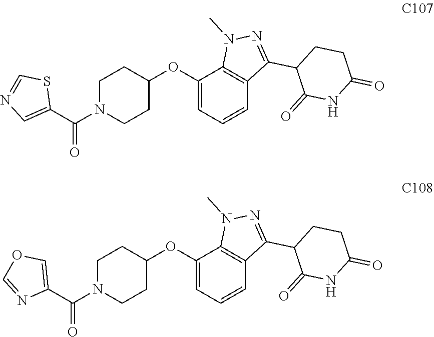 [SMILES: Cn1nc(C2CCC(=O)NC2=O)c2cccc(OC3CCN(C(=O)c4cncs4)CC3)c21 ; Cn1nc(C2CCC(=O)NC2=O)c2cccc(OC3CCN(C(=O)c4cocn4)CC3)c21]

(E)-3-(7-((1-(3-(4-Aminophenyl) acryloyl)piperidin-4-yl)oxy)-1-methyl-1H-indazol-3-yl)piperidine-2,6-dione C109. MS (ESI) m/z: 488.3 [M+1]⁺.

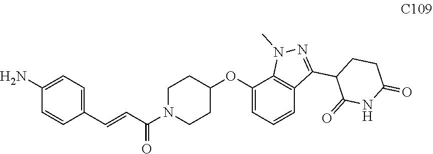 [SMILES: Cn1nc(C2CCC(=O)NC2=O)c2cccc(OC3CCN(C(=O)/C=C/c4ccc(N)cc4)CC3)c21]

3-(7-((1-(1-(6-(Cyclopropylamino)pyrimidin-4-yl)piperidine-4-carbonyl)-piperidin-4-yl)oxy)-1-methyl-1H-indazol-3-yl)piperidine-2,6-dione C110. MS (ESI) m/z: 587.3 [M+1]⁺.

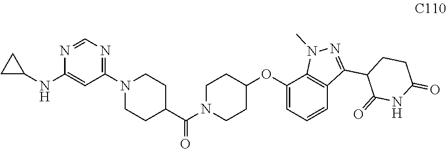 [SMILES: Cn1nc(C2CCC(=O)NC2=O)c2cccc(OC3CCN(C(=O)C4CCN(c5cc(NC6CC6)ncn5)CC4)CC3)c21]

3-(1-Methyl-7-((1-(oxazole-2-carbonyl)piperidin-4-yl)oxy)-1H-indazol-3-yl)-piperidine-2,6-dione C111. MS (ESI) m/z: 438.2 [M+1]⁺.

2-(4-((3-(2,6-Dioxopiperidin-3-yl)-1-methyl-1H-indazol-7-yl)oxy) piperidin-1-yl)-N-(4-methoxyphenyl)-2-oxoacetamide C112. MS (ESI) m/z: 520.2 [M+1]⁺.

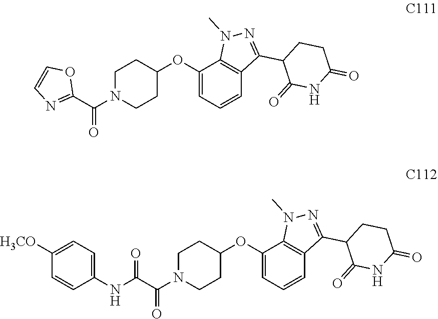 [SMILES: Cn1nc(C2CCC(=O)NC2=O)c2cccc(OC3CCN(C(=O)c4ncco4)CC3)c21 ; COc1ccc(NC(=O)C(=O)N2CCC(Oc3cccc4c(C5CCC(=O)NC5=O)nn(C)c34)CC2)cc1]

3-(7-((1-(1,2,4-Oxadiazole-3-carbonyl)piperidin-4-yl)oxy)-1-methyl-1H-indazol-3-yl)piperidine-2,6-dione C113. MS (ESI) m/z: 439.2 [M+1]⁺.

3-(1-Methyl-7-((1-(2-(piperazin-1-yl)acetyl)piperidin-4-yl)oxy)-1H-indazol-3-yl)-piperidine-2,6-dione C114. MS (ESI) m/z: 469.3 [M+1]⁺.

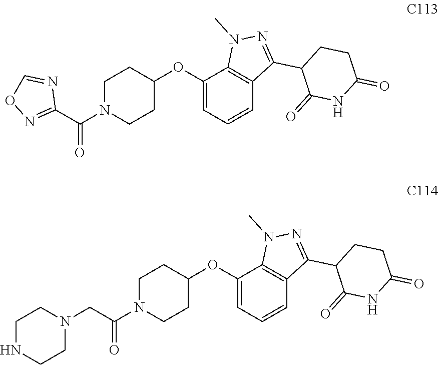 [SMILES: Cn1nc(C2CCC(=O)NC2=O)c2cccc(OC3CCN(C(=O)c4ncon4)CC3)c21 ; Cn1nc(C2CCC(=O)NC2=O)c2cccc(OC3CCN(C(=O)CN4CCNCC4)CC3)c21]

(E)-3-(1-Methyl-7-((1-(3-(thiazol-2-yl) acryloyl)piperidin-4-yl)oxy)-1H-indazol-3-yl)piperidine-2,6-dione C115. MS (ESI) m/z: 480.2 [M+1]⁺.

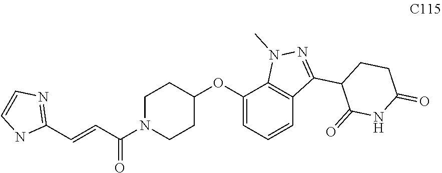 [SMILES: Cn1nc(C2CCC(=O)NC2=O)c2cccc(OC3CCN(C(=O)/C=C/c4ncc[nH]4)CC3)c21]

(E)-3-(1-Methyl-7-((1-(3-(5-methylthiophen-2-yl) acryloyl)piperidin-4-yl)oxy)-1H-indazol-3-yl)piperidine-2,6-dione C116. MS (ESI) m/z: 493.2 [M+1]⁺.

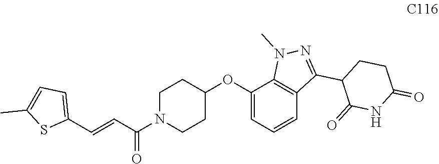 [SMILES: Cc1ccc(/C=C/C(=O)N2CCC(Oc3cccc4c(C5CCC(=O)NC5=O)nn(C)c34)CC2)s1]

1-(4-((3-(2,6-Dioxopiperidin-3-yl)-1-methyl-1H-indazol-7-yl)oxy) piperidine-1-carbonyl)cyclobutane-1-carbonitrile C117. MS (ESI) m/z: 450.2 [M+1]⁺.

3-(4-((3-(2,6-Dioxopiperidin-3-yl)-1-methyl-1H-indazol-7-yl)oxy) piperidine-1-carbonyl)-5-methylbenzonitrile C118. MS (ESI) m/z: 486.2 [M+1]⁺.

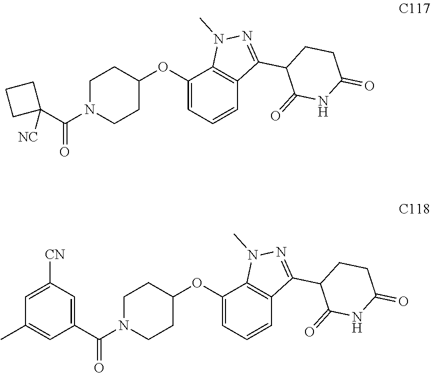 [SMILES: Cn1nc(C2CCC(=O)NC2=O)c2cccc(OC3CCN(C(=O)C4(C#N)CCC4)CC3)c21 ; Cc1cc(C#N)cc(C(=O)N2CCC(Oc3cccc4c(C5CCC(=O)NC5=O)nn(C)c34)CC2)c1]

2-(4-((3-(2,6-Dioxopiperidin-3-yl)-1-methyl-1H-indazol-7-yl)oxy) piperidin-1-yl)-N-(3-methoxyphenyl)-2-oxoacetamide C119. MS (ESI) m/z: 520.2 [M+1]⁺.

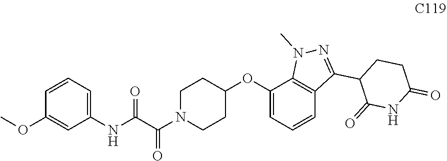 [SMILES: COc1cccc(NC(=O)C(=O)N2CCC(Oc3cccc4c(C5CCC(=O)NC5=O)nn(C)c34)CC2)c1]

3-(7-((1-(5-Chlorofuran-2-carbonyl)piperidin-4-yl)oxy)-1-methyl-1H-indazol-3-yl)piperidine-2,6-dione C120. MS (ESI) m/z: 471.1 [M+1]⁺.

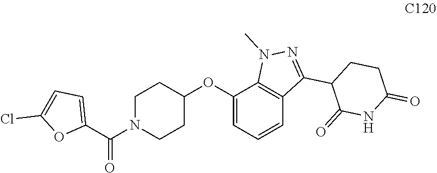 [SMILES: Cn1nc(C2CCC(=O)NC2=O)c2cccc(OC3CCN(C(=O)c4ccc(Cl)o4)CC3)c21]

3-(1-Methyl-7-((1-(2-(3-methylisoxazol-5-yl)acetyl)piperidin-4-yl)oxy)-1H-indazol-3-yl)piperidine-2,6-dione C121. MS (ESI) m/z: 466.2 [M+1]⁺.

(E)-3-(1-Methyl-7-((1-(pent-2-enoyl)piperidin-4-yl)oxy)-1H-indazol-3-yl)-piperidine-2,6-dione C122. MS (ESI) m/z: 425.2 [M+1]⁺.

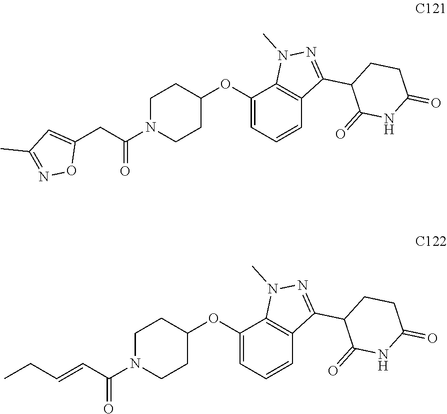 [SMILES: Cc1cc(CC(=O)N2CCC(Oc3cccc4c(C5CCC(=O)NC5=O)nn(C)c34)CC2)on1 ; CC/C=C/C(=O)N1CCC(Oc2cccc3c(C4CCC(=O)NC4=O)nn(C)c23)CC1]

(E)-3-(7-((1-(3-(Benzofuran-2-yl) acryloyl)piperidin-4-yl)oxy)-1-methyl-1H-indazol-3-yl)piperidine-2,6-dione C123. MS (ESI) m/z: 513.2 [M+1]⁺.

3-(1-Methyl-7-((1-(1-methylpiperidine-2-carbonyl)piperidin-4-yl)oxy)-1H-indazol-3-yl)piperidine-2,6-dione C124. MS (ESI) m/z: 468.3 [M+1]⁺.

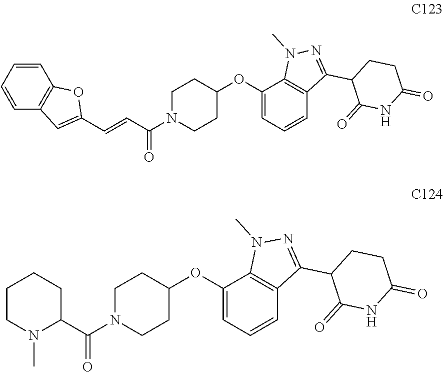 [SMILES: Cn1nc(C2CCC(=O)NC2=O)c2cccc(OC3CCN(C(=O)/C=C/c4cc5ccccc5o4)CC3)c21 ; CN1CCCCC1C(=O)N1CCC(Oc2cccc3c(C4CCC(=O)NC4=O)nn(C)c23)CC1]

3-(7-((1-(4-(1H-Pyrazol-1-yl)benzoyl)piperidin-4-yl)oxy)-1-methyl-1H-indazol-3-yl)piperidine-2,6-dione C125. MS (ESI) m/z: 513.2 [M+1]⁺.

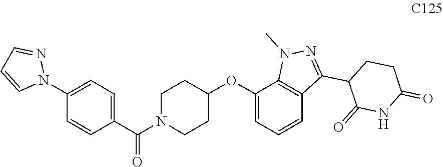 [SMILES: Cn1nc(C2CCC(=O)NC2=O)c2cccc(OC3CCN(C(=O)c4ccc(-n5cccn5)cc4)CC3)c21]

3-(7-((1-(2-(1,1-Dioxidothiomorpholino) acetyl)piperidin-4-yl)oxy)-1-methyl-1H-indazol-3-yl)piperidine-2,6-dione C126. MS (ESI) m/z: 518.2 [M+1]⁺.

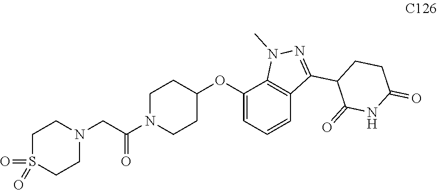 [SMILES: Cn1nc(C2CCC(=O)NC2=O)c2cccc(OC3CCN(C(=O)CN4CCS(=O)(=O)CC4)CC3)c21]

1-(3-(4-((3-(2,6-Dioxopiperidin-3-yl)-1-methyl-1H-indazol-7-yl)oxy) piperidin-1-yl)-3-oxopropyl) guanidine Cl₂₇. MS (ESI) m/z: 456.2 [M+1]⁺.

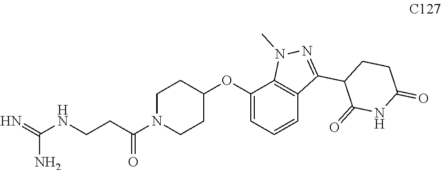 [SMILES: Cn1nc(C2CCC(=O)NC2=O)c2cccc(OC3CCN(C(=O)CCNC(=N)N)CC3)c21]

1-(2-(4-((3-(2,6-Dioxopiperidin-3-yl)-1-methyl-1H-indazol-7-yl)oxy) piperidin-1-yl)-2-oxoethyl) guanidine Cl₂₈. MS (ESI) m/z: 442.2 [M+1]⁺.

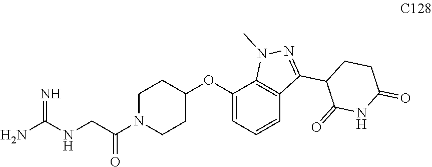 [SMILES: Cn1nc(C2CCC(=O)NC2=O)c2cccc(OC3CCN(C(=O)CNC(=N)N)CC3)c21]

3-(1-Methyl-7-((1-(4-methylthiazole-2-carbonyl)piperidin-4-yl)oxy)-1H-indazol-3-yl)piperidine-2,6-dione C129. MS (ESI) m/z: 468.2 [M+1]⁺.

3-(4-((3-(2,6-Dioxopiperidin-3-yl)-1-methyl-1H-indazol-7-yl)oxy) piperidine-1-carbonyl) bicyclo[1.1.1]pentane-1-carbonitrile C130. MS (ESI) m/z: 462.2 [M+1]⁺.

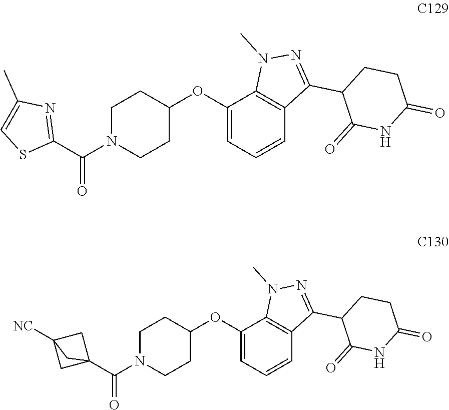 [SMILES: Cc1csc(C(=O)N2CCC(Oc3cccc4c(C5CCC(=O)NC5=O)nn(C)c34)CC2)n1 ; Cn1nc(C2CCC(=O)NC2=O)c2cccc(OC3CCN(C(=O)C45CC(C#N)(C4)C5)CC3)c21]

3-(7-((1-(2-Amino-4-methylthiazole-5-carbonyl)piperidin-4-yl)oxy)-1-methyl-1H-indazol-3-yl)piperidine-2,6-dione C131. MS (ESI) m/z: 483.2 [M+1]⁺.

3-(1-Methyl-7-((1-(4-nitrobenzoyl)piperidin-4-yl)oxy)-1H-indazol-3-yl)-piperidine-2,6-dione C132. MS (ESI) m/z: 492.2 [M+1]⁺.

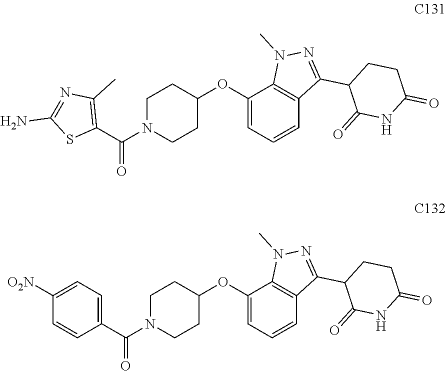 [SMILES: Cc1nc(N)sc1C(=O)N1CCC(Oc2cccc3c(C4CCC(=O)NC4=O)nn(C)c23)CC1 ; Cn1nc(C2CCC(=O)NC2=O)c2cccc(OC3CCN(C(=O)c4ccc([N+](=O)[O-])cc4)CC3)c21]

3-(7-((1-(1-(2-Fluorophenyl)-1H-pyrazole-4-carbonyl)piperidin-4-yl)oxy)-1-methyl-1H-indazol-3-yl)piperidine-2,6-dione C133. MS (ESI) m/z: 531.2 [M+1]⁺.

3-(1-Methyl-7-((1-(pyridazine-3-carbonyl)piperidin-4-yl)oxy)-1H-indazol-3-yl)-piperidine-2,6-dione C134. MS (ESI) m/z: 449.2 [M+1]⁺.

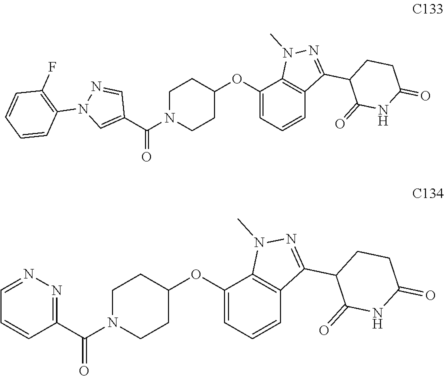 [SMILES: Cn1nc(C2CCC(=O)NC2=O)c2cccc(OC3CCN(C(=O)c4cnn(-c5ccccc5F)c4)CC3)c21 ; Cn1nc(C2CCC(=O)NC2=O)c2cccc(OC3CCN(C(=O)c4cccnn4)CC3)c21]

4-Amino-5-(4-((3-(2,6-dioxopiperidin-3-yl)-1-methyl-1H-indazol-7-yl)oxy)-piperidine-1-carbonyl)-N-ethylisothiazole-3-carboxamide C135. MS (ESI) m/z: 540.2 [M+1]⁺.

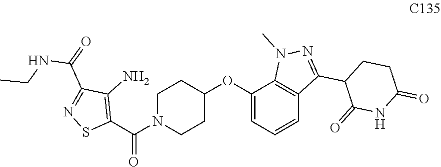 [SMILES: CCNC(=O)c1nsc(C(=O)N2CCC(Oc3cccc4c(C5CCC(=O)NC5=O)nn(C)c34)CC2)c1N]

4-Amino-5-(4-((3-(2,6-dioxopiperidin-3-yl)-1-methyl-1H-indazol-7-yl)oxy)-piperidine-1-carbonyl)-N-isopropylisothiazole-3-carboxamide C136. MS (ESI) m/z: 554.2 [M+1]⁺.

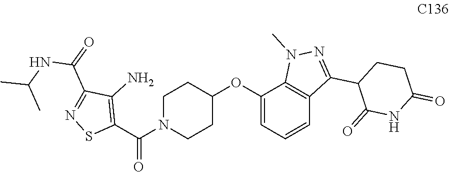 [SMILES: CC(C)NC(=O)c1nsc(C(=O)N2CCC(Oc3cccc4c(C5CCC(=O)NC5=O)nn(C)c34)CC2)c1N]

3-(1-Methyl-7-((1-(2-(trifluoromethoxy)benzoyl)piperidin-4-yl)oxy)-1H-indazol-3-yl)piperidine-2,6-dione C137. MS (ESI) m/z: 531.2 [M+1]⁺.

3-(7-((1-(2-(1H-Pyrazol-1-yl)acetyl)piperidin-4-yl)oxy)-1-methyl-1H-indazol-3-yl)piperidine-2,6-dione C138. MS (ESI) m/z: 451.2 [M+1]⁺.

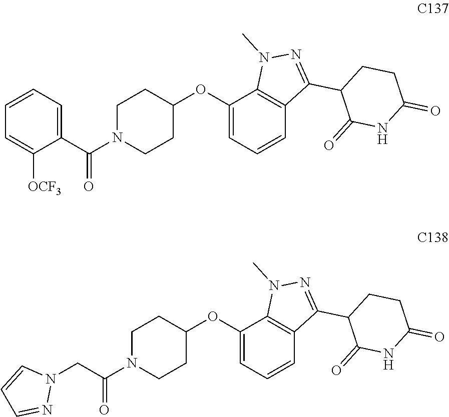 [SMILES: Cn1nc(C2CCC(=O)NC2=O)c2cccc(OC3CCN(C(=O)c4ccccc4OC(F)(F)F)CC3)c21 ; Cn1nc(C2CCC(=O)NC2=O)c2cccc(OC3CCN(C(=O)Cn4cccn4)CC3)c21]

3-(7-((1-(3-(Tert-butyl) bicyclo[1.1.1]pentane-1-carbonyl)piperidin-4-yl)oxy)-1-methyl-1H-indazol-3-yl)piperidine-2,6-dione C139. MS (ESI) m/z: 493.3 [M+1]⁺.

3-(1-Methyl-7-((1-(tetrahydro-2H-thiopyran-4-carbonyl)piperidin-4-yl)oxy)-1H-indazol-3-yl)piperidine-2,6-dione C140. MS (ESI) m/z: 471.2 [M+1]⁺.

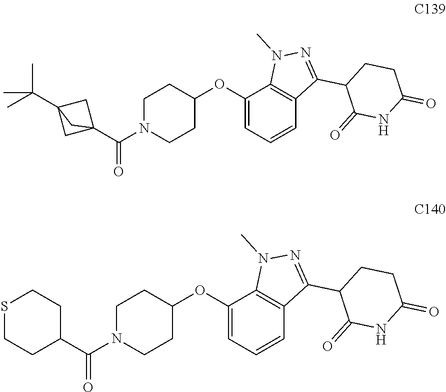 [SMILES: Cn1nc(C2CCC(=O)NC2=O)c2cccc(OC3CCN(C(=O)C45CC(C(C)(C)C)(C4)C5)CC3)c21 ; Cn1nc(C2CCC(=O)NC2=O)c2cccc(OC3CCN(C(=O)C4CCSCC4)CC3)c21]

3-(1-Methyl-7-((1-(1-methyl-5-(trifluoromethyl)-1H-imidazole-4-carbonyl)-piperidin-4-yl)oxy)-1H-indazol-3-yl)piperidine-2,6-dione C141. MS (ESI) m/z: 519.2 [M+1]⁺.

3-(7-((1-(2-(Benzyloxy)acetyl)piperidin-4-yl)oxy)-1-methyl-1H-indazol-3-yl)-piperidine-2,6-dione C142. MS (ESI) m/z: 491.2 [M+1]⁺.

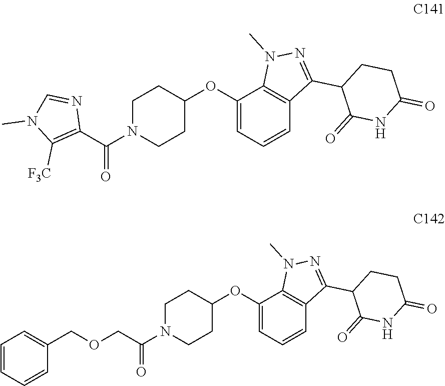 [SMILES: Cn1cnc(C(=O)N2CCC(Oc3cccc4c(C5CCC(=O)NC5=O)nn(C)c34)CC2)c1C(F)(F)F ; Cn1nc(C2CCC(=O)NC2=O)c2cccc(OC3CCN(C(=O)COCc4ccccc4)CC3)c21]

3-(7-((1-(1-Isopropyl-1H-pyrazole-5-carbonyl)piperidin-4-yl)oxy)-1-methyl-1H-indazol-3-yl)piperidine-2,6-dione C143. MS (ESI) m/z: 479.3 [M+1]⁺.

3-(1-Methyl-7-((1-(3-(trifluoromethoxy)benzoyl)piperidin-4-yl)oxy)-1H-indazol-3-yl)piperidine-2,6-dione C144. MS (ESI) m/z: 531.2 [M+1]⁺.

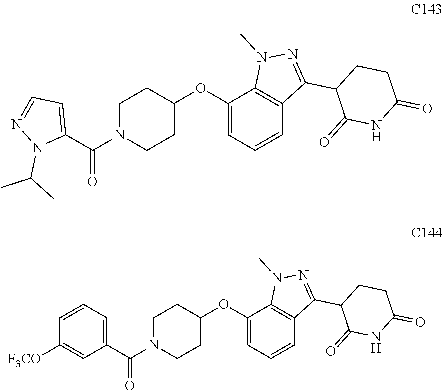 [SMILES: CC(C)n1nccc1C(=O)N1CCC(Oc2cccc3c(C4CCC(=O)NC4=O)nn(C)c23)CC1 ; Cn1nc(C2CCC(=O)NC2=O)c2cccc(OC3CCN(C(=O)c4cccc(OC(F)(F)F)c4)CC3)c21]

3-(1-Methyl-7-((1-(2-(thiophen-2-yl)acetyl)piperidin-4-yl)oxy)-1H-indazol-3-yl)-piperidine-2,6-dione C145. MS (ESI) m/z: 467.2 [M+1]⁺.

3-(1-Methyl-7-((1-(2-methyl-2,4,5,6-tetrahydrocyclopenta[c]pyrazole-3-carbonyl)piperidin-4-yl)oxy)-1H-indazol-3-yl)piperidine-2,6-dione C146. MS (ESI) m/z: 491.3 [M+1]⁺.

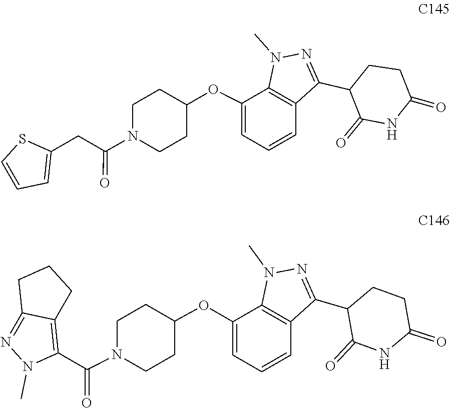 [SMILES: Cn1nc(C2CCC(=O)NC2=O)c2cccc(OC3CCN(C(=O)Cc4cccs4)CC3)c21 ; Cn1nc2c(c1C(=O)N1CCC(Oc3cccc4c(C5CCC(=O)NC5=O)nn(C)c34)CC1)CCC2]

3-(1-Methyl-7-((1-(thiophene-2-carbonyl)piperidin-4-yl)oxy)-1H-indazol-3-yl)-piperidine-2,6-dione C147. MS (ESI) m/z: 453.2 [M+1]⁺.

4-(4-((3-(2,6-Dioxopiperidin-3-yl)-1-methyl-1H-indazol-7-yl)oxy) piperidine-1-carbonyl)benzonitrile C148. MS (ESI) m/z: 472.2 [M+1]⁺.

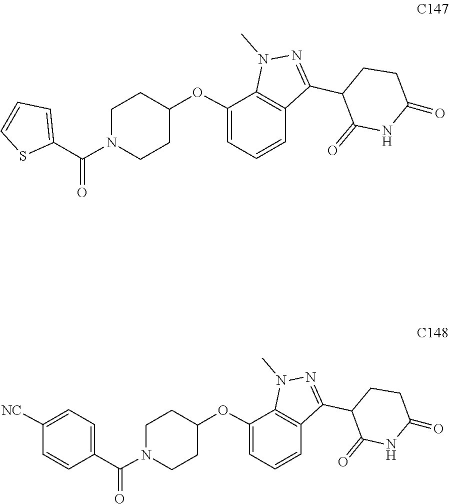 [SMILES: Cn1nc(C2CCC(=O)NC2=O)c2cccc(OC3CCN(C(=O)c4cccs4)CC3)c21 ; Cn1nc(C2CCC(=O)NC2=O)c2cccc(OC3CCN(C(=O)c4ccc(C#N)cc4)CC3)c21]

3-(7-((1-(Cyclopentanecarbonyl)piperidin-4-yl)oxy)-1-methyl-1H-indazol-3-yl)-piperidine-2,6-dione C149. MS (ESI) m/z: 439.2 [M+1]⁺.

3-(7-((1-(2-(Dimethylamino)pyrimidine-4-carbonyl)piperidin-4-yl)oxy)-1-methyl-1H-indazol-3-yl)piperidine-2,6-dione C150. MS (ESI) m/z: 492.2 [M+1]⁺.

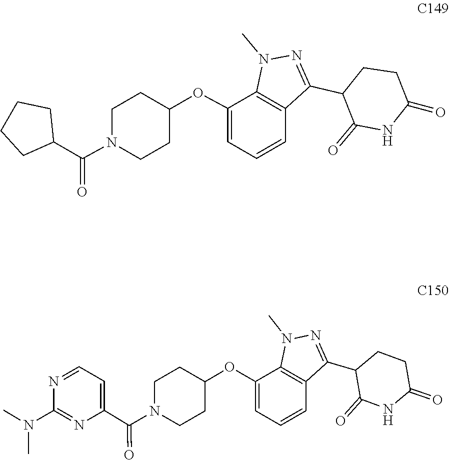 [SMILES: Cn1nc(C2CCC(=O)NC2=O)c2cccc(OC3CCN(C(=O)C4CCCC4)CC3)c21 ; CN(C)c1nccc(C(=O)N2CCC(Oc3cccc4c(C5CCC(=O)NC5=O)nn(C)c34)CC2)n1]

3-(7-((1-(1-(Tert-butyl)-1H-pyrazole-3-carbonyl)piperidin-4-yl)oxy)-1-methyl-1H-indazol-3-yl)piperidine-2,6-dione C151. MS (ESI) m/z: 493.3 [M+1]⁺.

(E)-3-(7-((1-Cinnamoylpiperidin-4-yl)oxy)-1-methyl-1H-indazol-3-yl)piperidine-2,6-dione C152. MS (ESI) m/z: 473.2 [M+1]⁺.

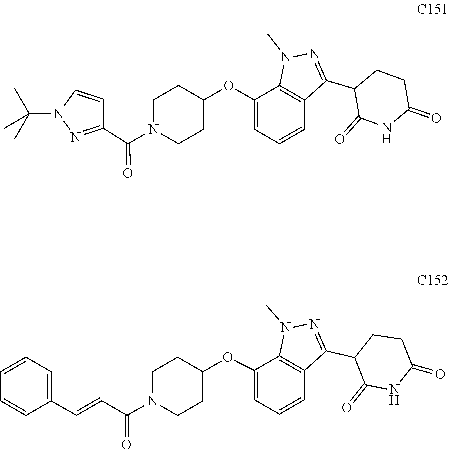 [SMILES: Cn1nc(C2CCC(=O)NC2=O)c2cccc(OC3CCN(C(=O)c4ccn(C(C)(C)C)n4)CC3)c21 ; Cn1nc(C2CCC(=O)NC2=O)c2cccc(OC3CCN(C(=O)/C=C/c4ccccc4)CC3)c21]

5-(2-(4-((3-(2,6-Dioxopiperidin-3-yl)-1-methyl-1H-indazol-7-yl)oxy) piperidin-1-yl)-2-oxoethyl)-1-methyl-1H-pyrrole-2-carbonitrile C153. MS (ESI) m/z: 489.2 [M+1]⁺.

3-(1-Methyl-7-((1-(thiazole-4-carbonyl)piperidin-4-yl)oxy)-1H-indazol-3-yl)-piperidine-2,6-dione C154. MS (ESI) m/z: 454.1 [M+1]⁺.

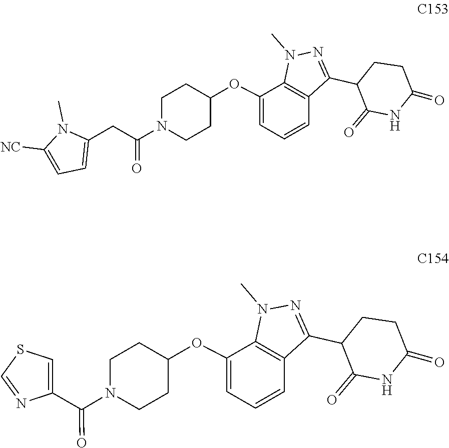 [SMILES: Cn1c(C#N)ccc1CC(=O)N1CCC(Oc2cccc3c(C4CCC(=O)NC4=O)nn(C)c23)CC1 ; Cn1nc(C2CCC(=O)NC2=O)c2cccc(OC3CCN(C(=O)c4cscn4)CC3)c21]

3-(1-Methyl-7-((1-(thiophene-3-carbonyl)piperidin-4-yl)oxy)-1H-indazol-3-yl)-piperidine-2,6-dione C155. MS (ESI) m/z: 453.1 [M+1]⁺.

3-(7-((1-(1-Acetylpiperidine-4-carbonyl)piperidin-4-yl)oxy)-1-methyl-1H-indazol-3-yl)piperidine-2,6-dione C156. MS (ESI) m/z: 496.3 [M+1]⁺.

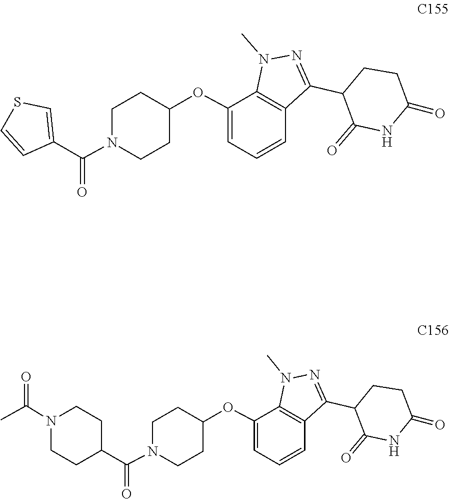 [SMILES: Cn1nc(C2CCC(=O)NC2=O)c2cccc(OC3CCN(C(=O)c4ccsc4)CC3)c21 ; CC(=O)N1CCC(C(=O)N2CCC(Oc3cccc4c(C5CCC(=O)NC5=O)nn(C)c34)CC2)CC1]

3-(7-((1-(3-Isopropoxybenzoyl)piperidin-4-yl)oxy)-1-methyl-1H-indazol-3-yl)-piperidine-2,6-dione C157. MS (ESI) m/z: 505.2 [M+1]⁺.

3-(1-Methyl-7-((1-(pyrimidine-4-carbonyl)piperidin-4-yl)oxy)-1H-indazol-3-yl)-piperidine-2,6-dione C158. MS (ESI) m/z: 449.2 [M+1]⁺.

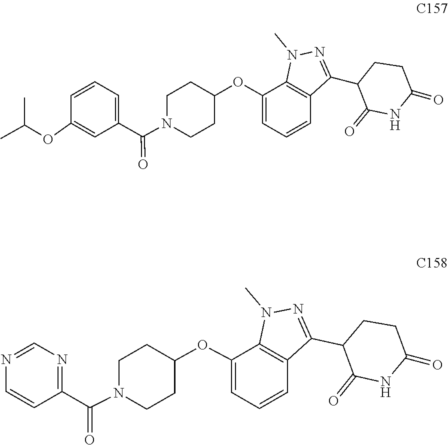 [SMILES: CC(C)Oc1cccc(C(=O)N2CCC(Oc3cccc4c(C5CCC(=O)NC5=O)nn(C)c34)CC2)c1 ; Cn1nc(C2CCC(=O)NC2=O)c2cccc(OC3CCN(C(=O)c4ccncn4)CC3)c21]

3-(1-Methyl-7-((1-(5-phenyloxazole-4-carbonyl)piperidin-4-yl)oxy)-1H-indazol-3-yl)piperidine-2,6-dione C159. MS (ESI) m/z: 514.2 [M+1]⁺.

3-(7-((1-(2,4-Dimethyloxazole-5-carbonyl)piperidin-4-yl)oxy)-1-methyl-1H-indazol-3-yl)piperidine-2,6-dione C160. MS (ESI) m/z: 466.2 [M+1]⁺.

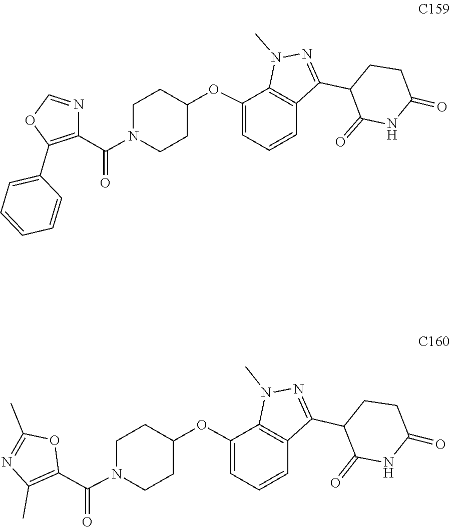 [SMILES: Cn1nc(C2CCC(=O)NC2=O)c2cccc(OC3CCN(C(=O)c4ncoc4-c4ccccc4)CC3)c21 ; Cc1nc(C)c(C(=O)N2CCC(Oc3cccc4c(C5CCC(=O)NC5=O)nn(C)c34)CC2)o1]

3-(1-Methyl-7-((1-(2-(phenylthio) acetyl)piperidin-4-yl)oxy)-1H-indazol-3-yl)-piperidine-2,6-dione C161. MS (ESI) m/z: 493.3 [M+1]⁺.

3-(1-Methyl-7-((1-(2-(1-methylpiperidin-4-yl)acetyl)piperidin-4-yl)oxy)-1H-indazol-3-yl)piperidine-2,6-dione C162. MS (ESI) m/z: 482.3 [M+1]⁺.

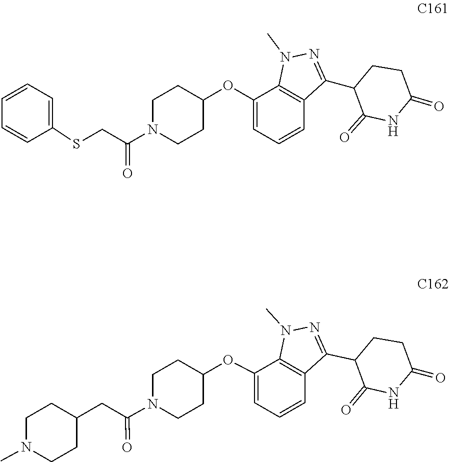 [SMILES: Cn1nc(C2CCC(=O)NC2=O)c2cccc(OC3CCN(C(=O)CSc4ccccc4)CC3)c21 ; CN1CCC(CC(=O)N2CCC(Oc3cccc4c(C5CCC(=O)NC5=O)nn(C)c34)CC2)CC1]

6-(4-((3-(2,6-Dioxopiperidin-3-yl)-1-methyl-1H-indazol-7-yl)oxy) piperidine-1-carbonyl) nicotinonitrile C163. MS (ESI) m/z: 473.1 [M+1]⁺.

3-(7-((1-(Cyclobutanecarbonyl)piperidin-4-yl)oxy)-1-methyl-1H-indazol-3-yl)-piperidine-2,6-dione C164. MS (ESI) m/z: 425.3 [M+1]⁺.

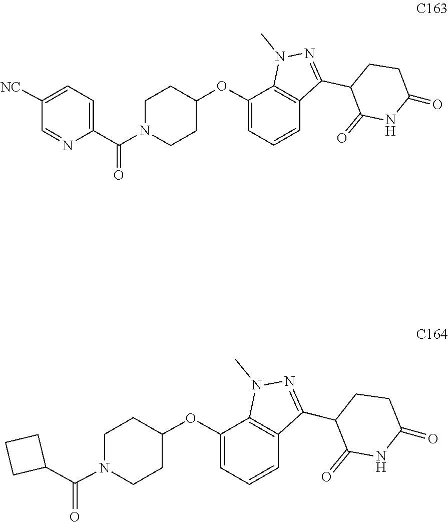 [SMILES: Cn1nc(C2CCC(=O)NC2=O)c2cccc(OC3CCN(C(=O)c4ccc(C#N)cn4)CC3)c21 ; Cn1nc(C2CCC(=O)NC2=O)c2cccc(OC3CCN(C(=O)C4CCC4)CC3)c21]

2-(3-(2,6-Dioxopiperidin-3-yl)-1H-indazol-1-yl)-N-(4-(methylsulfonyl)phenyl)-acetamide D002. MS (ESI) m/z: 441 [M+1]⁺.

2-(3-(2,6-Dioxopiperidin-3-yl)-1H-indazol-1-yl)-N-(1-(tetrahydro-2H-pyran-4-yl)-1H-pyrazol-4-yl)acetamide D004. MS (ESI) m/z: 437.1 [M+1]⁺.

2-(3-(2,6-Dioxopiperidin-3-yl)-1H-indazol-1-yl)-N-(3-ethylisoxazol-5-yl)-acetamide D005. MS (ESI) m/z: 382.1 [M+1]⁺.

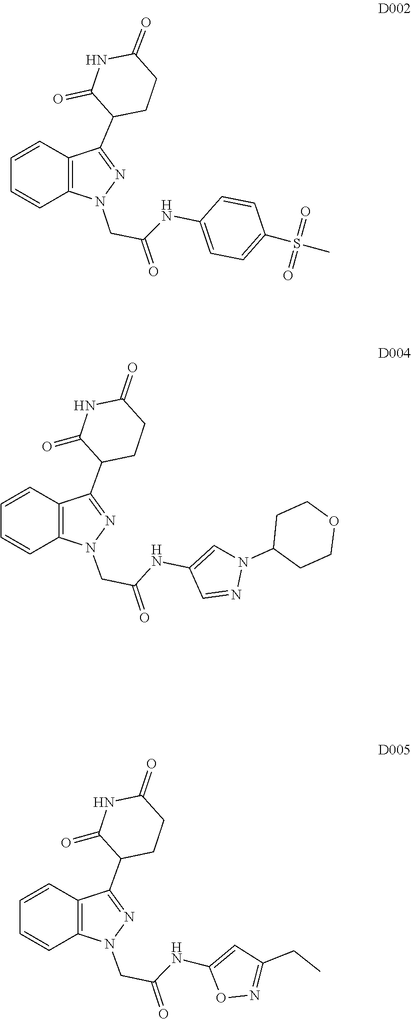 [SMILES: CS(=O)(=O)c1ccc(NC(=O)Cn2nc(C3CCC(=O)NC3=O)c3ccccc32)cc1 ; O=C1CCC(c2nn(CC(=O)Nc3cnn(C4CCOCC4)c3)c3ccccc23)C(=O)N1 ; CCc1cc(NC(=O)Cn2nc(C3CCC(=O)NC3=O)c3ccccc32)on1]

2-(3-(2,6-Dioxopiperidin-3-yl)-1H-indazol-1-yl)-N-(1-methyl-3-(trifluoromethyl)-1H-pyrazol-4-yl)acetamide D006. MS (ESI) m/z: 435 [M+1]⁺.

2-(3-(2,6-Dioxopiperidin-3-yl)-1H-indazol-1-yl)-N-(thiazol-4-yl)acetamide D007. MS (ESI) m/z: 370.1 [M+1]⁺.

2-(3-(2,6-Dioxopiperidin-3-yl)-1H-indazol-1-yl)-N-(4-(N-methylsulfamoyl)-phenyl)acetamide D008. MS (ESI) m/z: 456 [M+1]⁺.

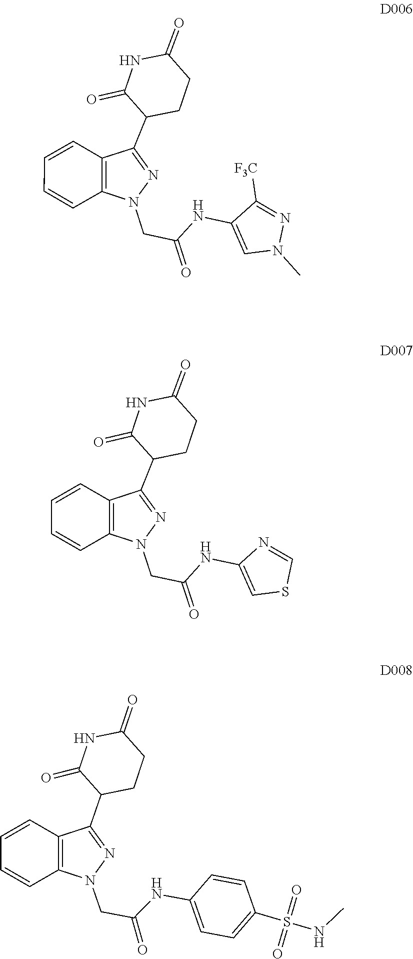 [SMILES: Cn1cc(NC(=O)Cn2nc(C3CCC(=O)NC3=O)c3ccccc32)c(C(F)(F)F)n1 ; O=C1CCC(c2nn(CC(=O)Nc3cscn3)c3ccccc23)C(=O)N1 ; CNS(=O)(=O)c1ccc(NC(=O)Cn2nc(C3CCC(=O)NC3=O)c3ccccc32)cc1]

2-(3-(2,6-Dioxopiperidin-3-yl)-1H-indazol-1-yl)-N-((1-hydroxycyclopropyl)-methyl)acetamide D009. MS (ESI) m/z: 357.2 [M+1]⁺.

2-(3-(2,6-Dioxopiperidin-3-yl)-1H-indazol-1-yl)-N-(2-methylthiazol-5-yl)-acetamide DO10. MS (ESI) m/z: 384.1 [M+1]⁺.

2-(3-(2,6-Dioxopiperidin-3-yl)-1H-indazol-1-yl)-N-(3-methylisothiazol-5-yl)-acetamide DO11. MS (ESI) m/z: 384.1 [M+1]⁺.

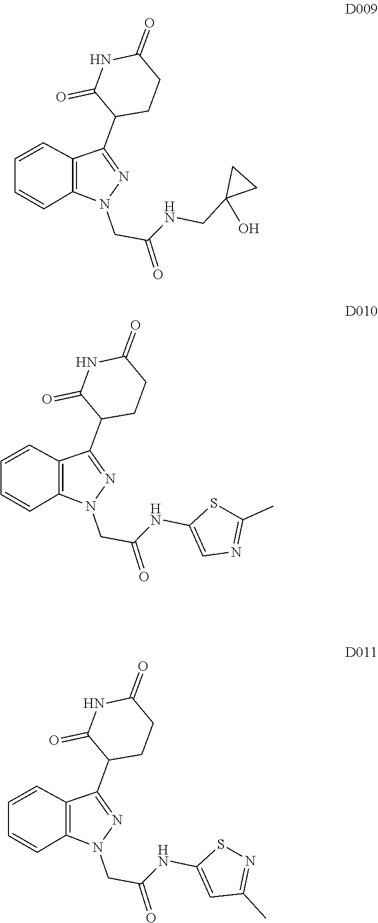 [SMILES: O=C(Cn1nc(C2CCC(=O)NC2=O)c2ccccc21)NCC1(O)CC1 ; Cc1ncc(NC(=O)Cn2nc(C3CCC(=O)NC3=O)c3ccccc32)s1 ; Cc1cc(NC(=O)Cn2nc(C3CCC(=O)NC3=O)c3ccccc32)sn1]

2-(3-(2,6-Dioxopiperidin-3-yl)-1H-indazol-1-yl)-N-(pyridazin-3-yl)acetamide DO12. MS (ESI) m/z: 365.2 [M+1]⁺.

N-(3-Cyclopropyl-1-methyl-1H-pyrazol-5-yl)-2-(3-(2,6-dioxopiperidin-3-yl)-1H-indazol-1-yl)acetamide D013. MS (ESI) m/z: 407.2 [M+1]⁺.

N-(3,5-Dimethylisoxazol-4-yl)-2-(3-(2,6-dioxopiperidin-3-yl)-1H-indazol-1-yl)-acetamide D014. MS (ESI) m/z: 382.2 [M+1]⁺.

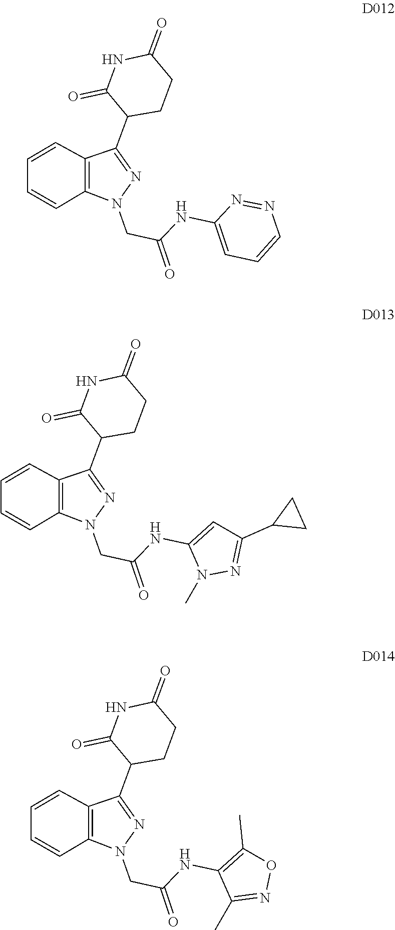 [SMILES: O=C1CCC(c2nn(CC(=O)Nc3cccnn3)c3ccccc23)C(=O)N1 ; Cn1nc(C2CC2)cc1NC(=O)Cn1nc(C2CCC(=O)NC2=O)c2ccccc21 ; Cc1noc(C)c1NC(=O)Cn1nc(C2CCC(=O)NC2=O)c2ccccc21]

2-(3-(2,6-Dioxopiperidin-3-yl)-1H-indazol-1-yl)-N-(1-isopropyl-1H-pyrazol-4-yl)-acetamide D015. MS (ESI) m/z: 395.2 [M+1]⁺.

N-(3-(Tert-butyl)-1-phenyl-1H-pyrazol-5-yl)-2-(3-(2,6-dioxopiperidin-3-yl)-1H-indazol-1-yl)acetamide D016. MS (ESI) m/z: 485.2 [M+1]⁺.

2-(3-(2,6-Dioxopiperidin-3-yl)-1H-indazol-1-yl)-N-(3-methylisoxazol-5-yl)-acetamide D017. MS (ESI) m/z: 368.2 [M+1]⁺.

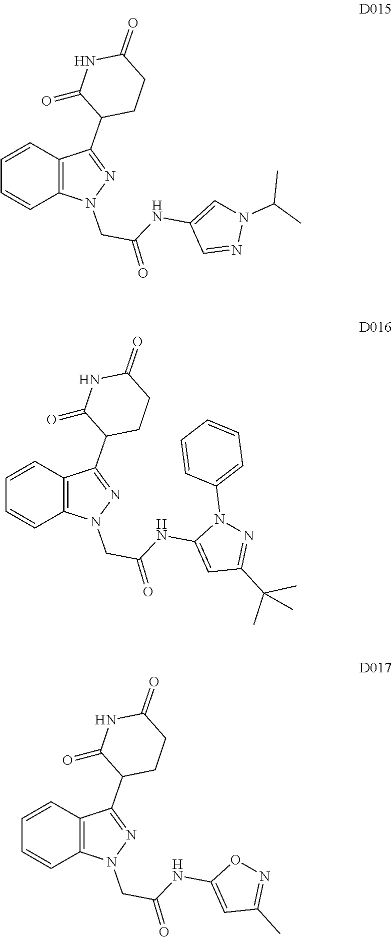 [SMILES: CC(C)n1cc(NC(=O)Cn2nc(C3CCC(=O)NC3=O)c3ccccc32)cn1 ; CC(C)(C)c1cc(NC(=O)Cn2nc(C3CCC(=O)NC3=O)c3ccccc32)n(-c2ccccc2)n1 ; Cc1cc(NC(=O)Cn2nc(C3CCC(=O)NC3=O)c3ccccc32)on1]

2-(3-(2,6-Dioxopiperidin-3-yl)-1H-indazol-1-yl)-N-(4-(oxazol-5-yl)phenyl)-acetamide D018. MS (ESI) m/z: 430.2 [M+1]⁺.

N-(1-(Tert-butyl)-3-methyl-1H-pyrazol-5-yl)-2-(3-(2,6-dioxopiperidin-3-yl)-1H-indazol-1-yl)acetamide D019. MS (ESI) m/z: 423.2 [M+1]⁺.

N-(3,5-Dimethyl-1H-pyrazol-4-yl)-2-(3-(2,6-dioxopiperidin-3-yl)-1H-indazol-1-yl)acetamide D020. MS (ESI) m/z: 381.1 [M+1]⁺.

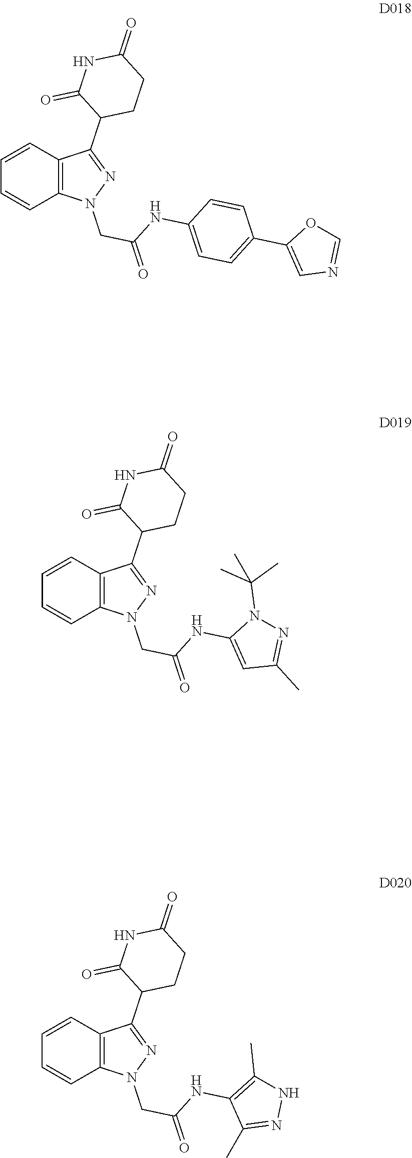 [SMILES: O=C1CCC(c2nn(CC(=O)Nc3ccc(-c4cnco4)cc3)c3ccccc23)C(=O)N1 ; Cc1cc(NC(=O)Cn2nc(C3CCC(=O)NC3=O)c3ccccc32)n(C(C)(C)C)n1 ; Cc1n[nH]c(C)c1NC(=O)Cn1nc(C2CCC(=O)NC2=O)c2ccccc21]

2-(3-(2,6-Dioxopiperidin-3-yl)-1H-indazol-1-yl)-N-(3-methylisothiazol-4-yl)-acetamide D021. MS (ESI) m/z: 384.1 [M+1]⁺.

2-(3-(2,6-Dioxopiperidin-3-yl)-1H-indazol-1-yl)-N-(4-(2-methyl-1H-imidazol-1-yl)phenyl)acetamide D022. MS (ESI) m/z: 443.1 [M+1]⁺.

2-(3-(2,6-Dioxopiperidin-3-yl)-1H-indazol-1-yl)-N-(4-(4-methylpiperazin-1-yl)-phenyl)acetamide D023. MS (ESI) m/z: 461.1 [M+1]⁺.

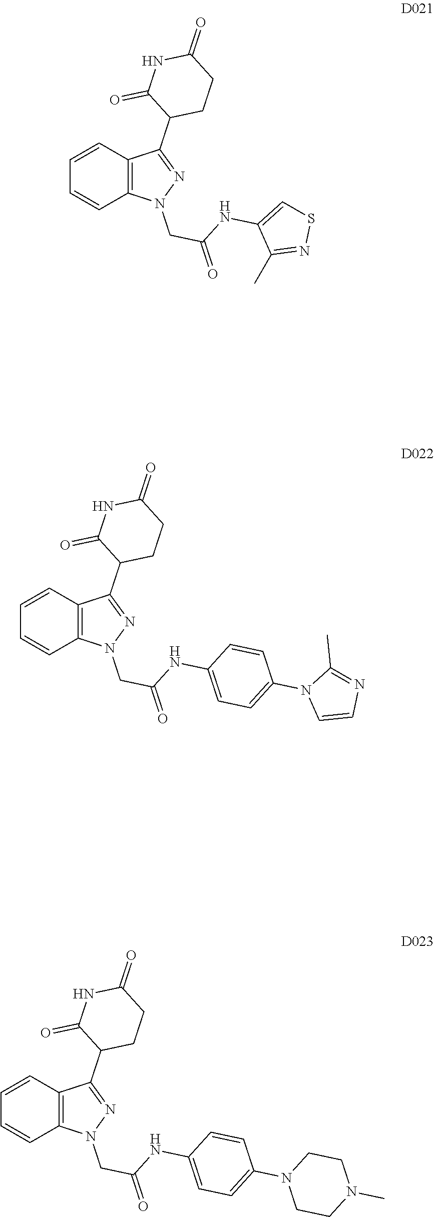 [SMILES: Cc1nscc1NC(=O)Cn1nc(C2CCC(=O)NC2=O)c2ccccc21 ; Cc1nccn1-c1ccc(NC(=O)Cn2nc(C3CCC(=O)NC3=O)c3ccccc32)cc1 ; CN1CCN(c2ccc(NC(=O)Cn3nc(C4CCC(=O)NC4=O)c4ccccc43)cc2)CC1]

2-(3-(2,6-Dioxopiperidin-3-yl)-1H-indazol-1-yl)-N-(1-methyl-1H-pyrazol-5-yl)-acetamide DO24. MS (ESI) m/z: 367.2 [M+1]⁺.

N-(4-(1H-Imidazol-2-yl)phenyl)-2-(3-(2,6-dioxopiperidin-3-yl)-1H-indazol-1-yl)-acetamide D025. MS (ESI) m/z: 429.2 [M+1]⁺.

N-(3-(1H-Imidazol-1-yl)phenyl)-2-(3-(2,6-dioxopiperidin-3-yl)-1H-indazol-1-yl)-acetamide D026. MS (ESI) m/z: 429.2 [M+1]⁺.

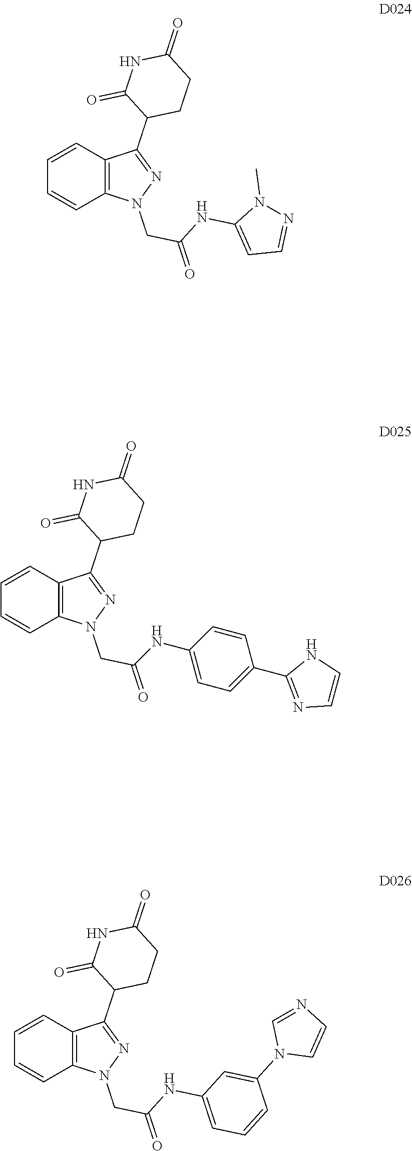 [SMILES: Cn1nccc1NC(=O)Cn1nc(C2CCC(=O)NC2=O)c2ccccc21 ; O=C1CCC(c2nn(CC(=O)Nc3ccc(-c4ncc[nH]4)cc3)c3ccccc23)C(=O)N1 ; O=C1CCC(c2nn(CC(=O)Nc3cccc(-n4ccnc4)c3)c3ccccc23)C(=O)N1]

2-(3-(2,6-Dioxopiperidin-3-yl)-1H-indazol-1-yl)-N-((1-methyl-1H-pyrazol-3-yl)-methyl)acetamide D027. MS (ESI) m/z: 381.2 [M+1]⁺.

2-(3-(2,6-Dioxopiperidin-3-yl)-1H-indazol-1-yl)-N-((1-methyl-1H-imidazol-5-yl)methyl)acetamide D028. MS (ESI) m/z: 381.2 [M+1]⁺.

2-(3-(2,6-Dioxopiperidin-3-yl)-1H-indazol-1-yl)-N-(1-methylpiperidin-4-yl)-acetamide D029. MS (ESI) m/z: 384.2 [M+1]⁺.

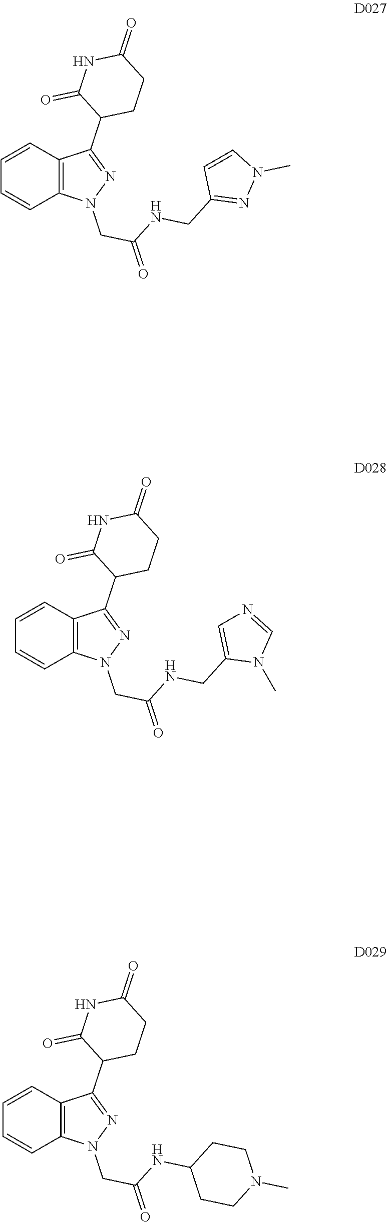 [SMILES: Cn1ccc(CNC(=O)Cn2nc(C3CCC(=O)NC3=O)c3ccccc32)n1 ; Cn1cncc1CNC(=O)Cn1nc(C2CCC(=O)NC2=O)c2ccccc21 ; CN1CCC(NC(=O)Cn2nc(C3CCC(=O)NC3=O)c3ccccc32)CC1]

2-(3-(2,6-Dioxopiperidin-3-yl)-1H-indazol-1-yl)-N-(3-hydroxycyclopentyl)-acetamide DO30. MS (ESI) m/z: 371.2 [M+1]⁺.

N-(1,1-Dioxidothietan-3-yl)-2-(3-(2,6-dioxopiperidin-3-yl)-1H-indazol-1-yl)-acetamide DO31. MS (ESI) m/z: 391.1 [M+1]⁺.

N-(3-(Tert-butyl)phenyl)-2-(3-(2,6-dioxopiperidin-3-yl)-1H-indazol-1-yl)-acetamide D032. MS (ESI) m/z: 419.2 [M+1]⁺.

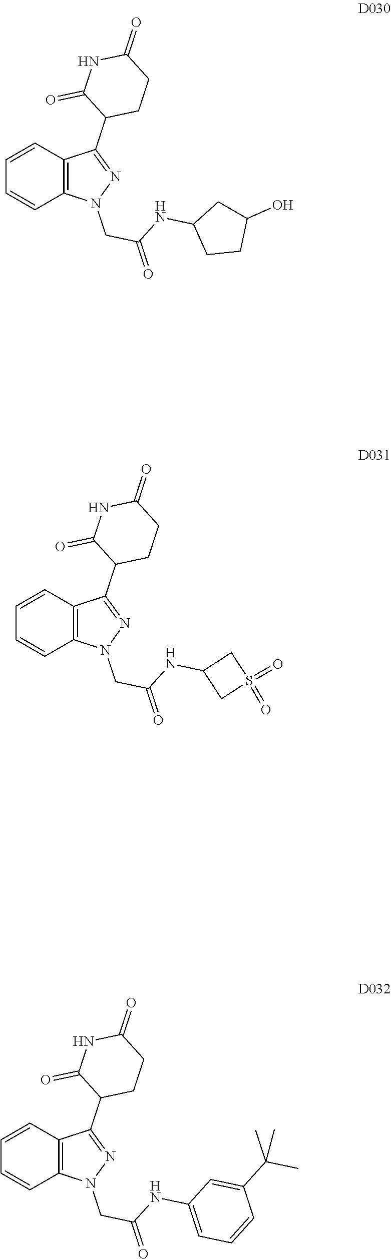 [SMILES: O=C1CCC(c2nn(CC(=O)NC3CCC(O)C3)c3ccccc23)C(=O)N1 ; O=C1CCC(c2nn(CC(=O)NC3CS(=O)(=O)C3)c3ccccc23)C(=O)N1 ; CC(C)(C)c1cccc(NC(=O)Cn2nc(C3CCC(=O)NC3=O)c3ccccc32)c1]

N-(2-(Benzyloxy)ethyl)-2-(3-(2,6-dioxopiperidin-3-yl)-1H-indazol-1-yl)-acetamide D033. MS (ESI) m/z: 421.1 [M+1]⁺.

2-(3-(2,6-Dioxopiperidin-3-yl)-1H-indazol-1-yl)-N-(3-(oxazol-5-yl)phenyl)-acetamide D034. MS (ESI) m/z: 430.2 [M+1]⁺.

2-(3-(2,6-Dioxopiperidin-3-yl)-1H-indazol-1-yl)-N-(3-isopropoxyphenyl)-acetamide D035. MS (ESI) m/z: 421.2 [M+1]⁺.

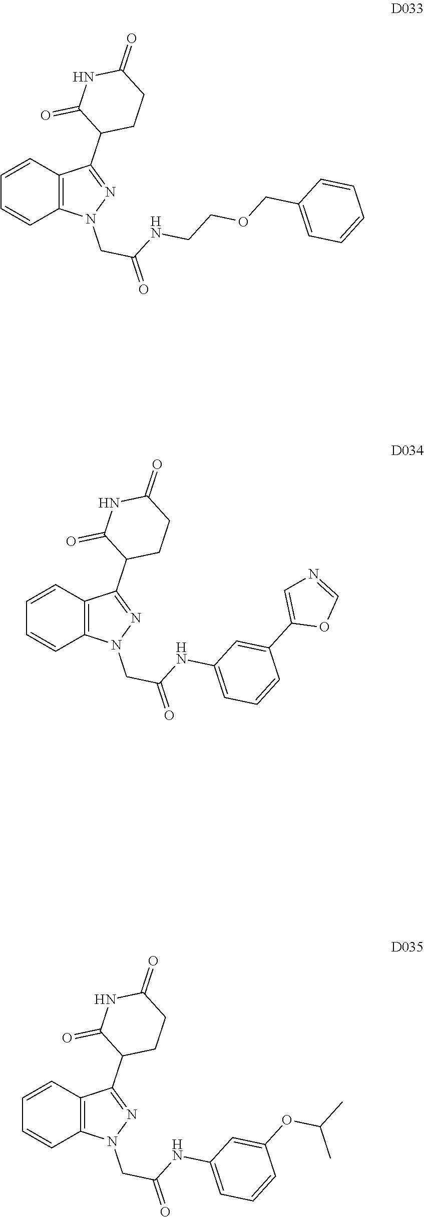 [SMILES: O=C(Cn1nc(C2CCC(=O)NC2=O)c2ccccc21)NCCOCc1ccccc1 ; O=C1CCC(c2nn(CC(=O)Nc3cccc(-c4cnco4)c3)c3ccccc23)C(=O)N1 ; CC(C)Oc1cccc(NC(=O)Cn2nc(C3CCC(=O)NC3=O)c3ccccc32)c1]

2-(3-(2,6-Dioxopiperidin-3-yl)-1H-indazol-1-yl)-N-(4-(trifluoromethoxy)phenyl)-acetamide D036. MS (ESI) m/z: 447.2 [M+1]⁺.

2-(3-(2,6-Dioxopiperidin-3-yl)-1H-indazol-1-yl)-N-(4-isopropylbenzyl)acetamide D037. MS (ESI) m/z: 419.2 [M+1]⁺.

2-(3-(2,6-Dioxopiperidin-3-yl)-1H-indazol-1-yl)-N-(4-hydroxybenzyl)acetamide D038. MS (ESI) m/z: 393.1 [M+1]⁺.

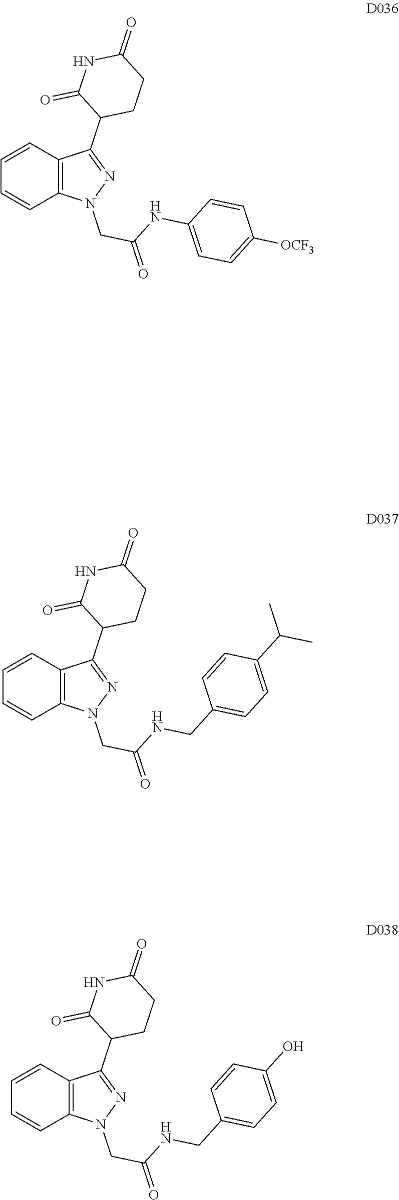 [SMILES: O=C1CCC(c2nn(CC(=O)Nc3ccc(OC(F)(F)F)cc3)c3ccccc23)C(=O)N1 ; CC(C)c1ccc(CNC(=O)Cn2nc(C3CCC(=O)NC3=O)c3ccccc32)cc1 ; O=C(Cn1nc(C2CCC(=O)NC2=O)c2ccccc21)NCc1ccc(O)cc1]

2-(3-(2,6-dioxopiperidin-3-yl)-1H-indazol-1-yl)-N-(4-methyltetrahydro-2H-pyran-4-yl)acetamide D039. MS (ESI) m/z: 385.1 [M+1]⁺.

N-Cyclohexyl-2-(3-(2,6-dioxopiperidin-3-yl)-1H-indazol-1-yl)acetamide D040. MS (ESI) m/z: 369.1 [M+1]⁺.

2-(3-(2,6-Dioxopiperidin-3-yl)-1H-indazol-1-yl)-N-(thiophen-2-ylmethyl)-acetamide D041. MS (ESI) m/z: 383.2 [M+1]⁺.

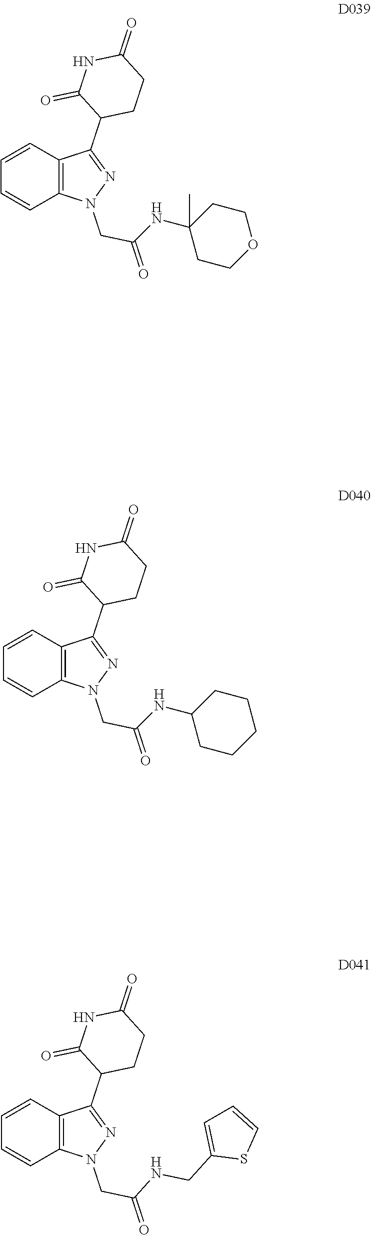 [SMILES: CC1(NC(=O)Cn2nc(C3CCC(=O)NC3=O)c3ccccc32)CCOCC1 ; O=C1CCC(c2nn(CC(=O)NC3CCCCC3)c3ccccc23)C(=O)N1 ; O=C(Cn1nc(C2CCC(=O)NC2=O)c2ccccc21)NCc1cccs1]

2-(3-(2,6-Dioxopiperidin-3-yl)-1H-indazol-1-yl)-N-((3-hydroxycyclopentyl)-methyl)acetamide D042. MS (ESI) m/z: 385.2 [M+1]⁺.

2-(3-(2,6-Dioxopiperidin-3-yl)-1H-indazol-1-yl)-N-(tetrahydro-2H-pyran-4-yl)-acetamide D043. MS (ESI) m/z: 371.1 [M+1]⁺.

N-(2-(Cyclopentyloxy)ethyl)-2-(3-(2,6-dioxopiperidin-3-yl)-1H-indazol-1-yl)-acetamide D044. MS (ESI) m/z: 399.1 [M+1]⁺.

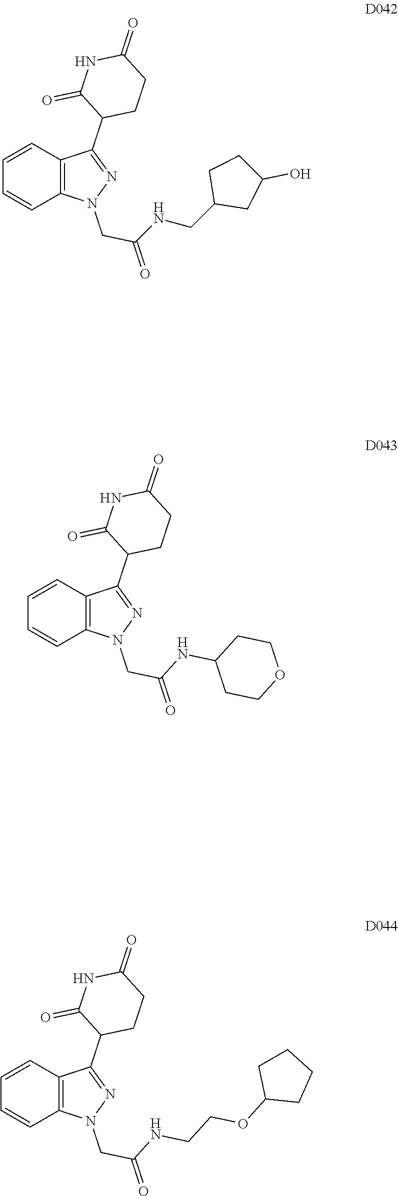 [SMILES: O=C(Cn1nc(C2CCC(=O)NC2=O)c2ccccc21)NCC1CCC(O)C1 ; O=C1CCC(c2nn(CC(=O)NC3CCOCC3)c3ccccc23)C(=O)N1 ; O=C(Cn1nc(C2CCC(=O)NC2=O)c2ccccc21)NCCOC1CCCC1]

2-(3-(2,6-Dioxopiperidin-3-yl)-1H-indazol-1-yl)-N-(3-fluorobicyclo[1.1.1]pentan-1-yl)acetamide D045. MS (ESI) m/z: 371.2 [M+1]⁺.

2-(3-(2,6-Dioxopiperidin-3-yl)-1H-indazol-1-yl)-N-(4-(p-tolyloxy)phenyl)-acetamide D046. MS (ESI) m/z: 469.2 [M+1]⁺.

2-(3-(2,6-Dioxopiperidin-3-yl)-1H-indazol-1-yl)-N-(thiazol-2-ylmethyl)acetamide D047. MS (ESI) m/z: 384.1 [M+1]⁺.

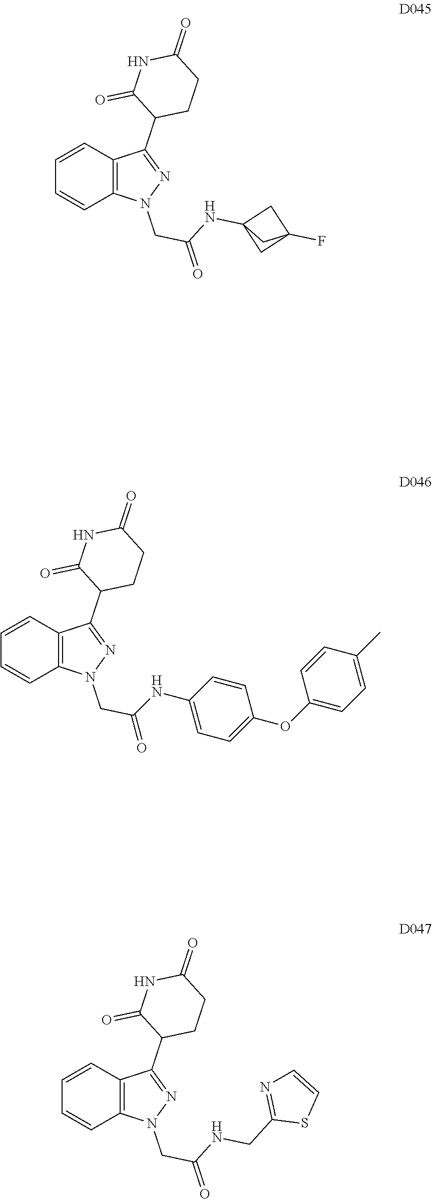 [SMILES: O=C1CCC(c2nn(CC(=O)NC34CC(F)(C3)C4)c3ccccc23)C(=O)N1 ; Cc1ccc(Oc2ccc(NC(=O)Cn3nc(C4CCC(=O)NC4=O)c4ccccc43)cc2)cc1 ; O=C(Cn1nc(C2CCC(=O)NC2=O)c2ccccc21)NCc1nccs1]

N-(Cyclopropylmethyl)-2-(3-(2,6-dioxopiperidin-3-yl)-1H-indazol-1-yl)-acetamide D048. MS (ESI) m/z: 341.2 [M+1]⁺.

2-(3-(2,6-Dioxopiperidin-3-yl)-1H-indazol-1-yl)-N-(1-methylazetidin-3-yl)-acetamide D050. MS (ESI) m/z: 356.2 [M+1]⁺.

N-(Cyclopentylmethyl)-2-(3-(2,6-dioxopiperidin-3-yl)-1H-indazol-1-yl)acetamide D051. MS (ESI) m/z: 369.2 [M+1]⁺.

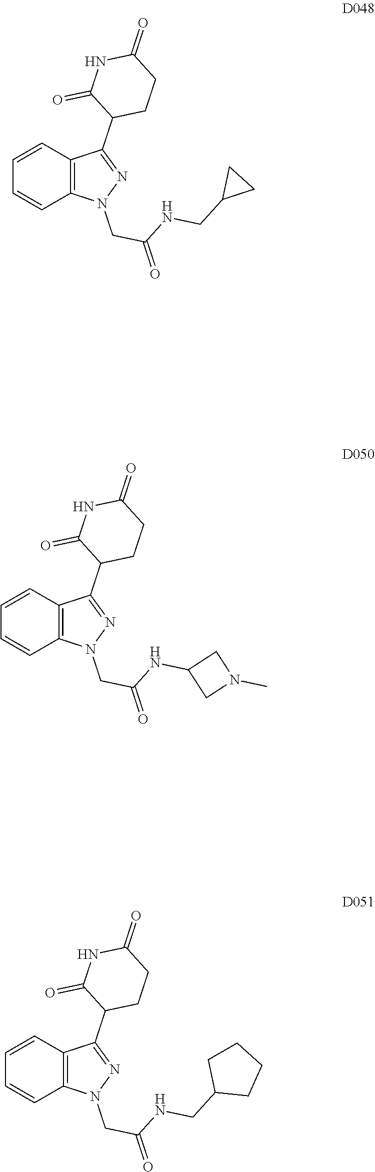 [SMILES: O=C(Cn1nc(C2CCC(=O)NC2=O)c2ccccc21)NCC1CC1 ; CN1CC(NC(=O)Cn2nc(C3CCC(=O)NC3=O)c3ccccc32)C1 ; O=C(Cn1nc(C2CCC(=O)NC2=O)c2ccccc21)NCC1CCCC1]

2-(3-(2,6-Dioxopiperidin-3-yl)-1H-indazol-1-yl)-N-neopentylacetamide D052. MS (ESI) m/z: 357.3 [M+1]⁺.

2-(3-(2,6-Dioxopiperidin-3-yl)-1H-indazol-1-yl)-N-(tetrahydrofuran-3-yl)-acetamide D053. MS (ESI) m/z: 357.2 [M+1]⁺.

N-Cyclopentyl-2-(3-(2,6-dioxopiperidin-3-yl)-1H-indazol-1-yl)acetamide D054. MS (ESI) m/z: 355.2 [M+1]⁺.

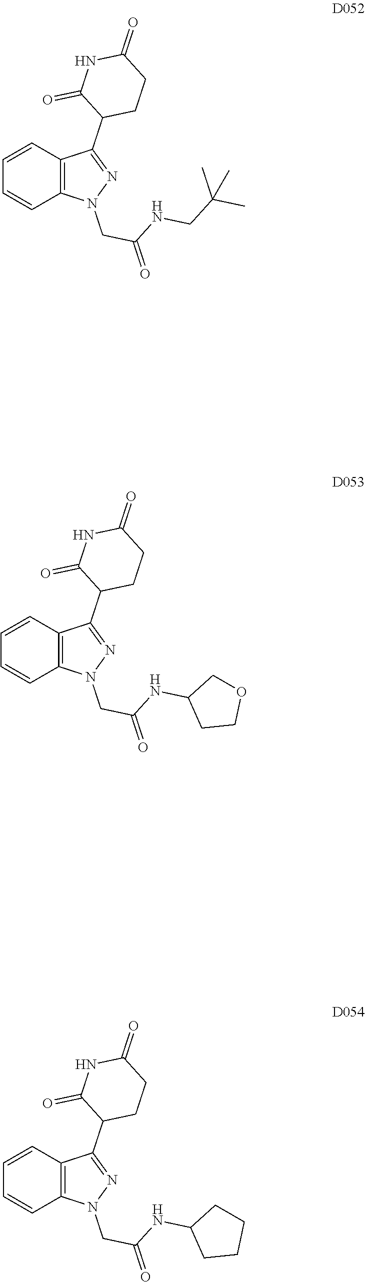 [SMILES: CC(C)(C)CNC(=O)Cn1nc(C2CCC(=O)NC2=O)c2ccccc21 ; O=C1CCC(c2nn(CC(=O)NC3CCOC3)c3ccccc23)C(=O)N1 ; O=C1CCC(c2nn(CC(=O)NC3CCCC3)c3ccccc23)C(=O)N1]

2-(3-(2,6-Dioxopiperidin-3-yl)-1H-indazol-1-yl)-N-(2-(methylamino)-2-oxo-ethyl)acetamide D055. MS (ESI) m z: 358.2 [M+1]⁺.

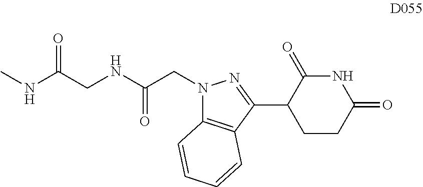 [SMILES: CNC(=O)CNC(=O)Cn1nc(C2CCC(=O)NC2=O)c2ccccc21]

### Example B1

### Protein Degradation Assays

A compound provided herein is assessed for its activity in degrading CK1α, GSPT1, and Ikaros in cancerous cells (e.g., MV-4-11 cells) via a western blot. The cells are grown at 37° C. in a humidified 5% CO₂ environment in RPMI 1640 media supplemented with 10% fetal bovine serum, streptomycin, and penicillin. The cells are cultured at approximately 10⁶ cells per mL. The cells are treated with a compound or DMSO (control) for about 8 h. Whole cell extracts are prepared using a radioimmunoprecipitation assay (RIPA) buffer. Briefly, 3×10⁶ cells are washed once in PBS, and the cell pellets are resuspended in the RIPA buffer and incubated for 15 min on ice. Cells debris is removed by centrifugation and the cleared whole cell lysates are transferred to new tubes for further analysis.

For a western blot analysis, the whole cell protein extracts are separated on 12% SDS-polyacrylamide gels, transferred to nitrocellulose, and probed with primary antibodies, including an anti-hPD-L¹ antibody. Membranes are subsequently washed and probed with IRDYER secondary antibodies. The signals are detected using an ODYSSEY® Imaging System.

For Western blot analysis, the whole cell protein extracts are separated on 4-12% SDS-polyacrylamide gels, transferred to nitrocellulose, and probed with primary antibodies, including anti-eRF3/GSPT1, anti-Ikaros, anti-CK1α, and β-actin (8H10D10) mouse monoclonal antibody. Membranes are subsequently washed and probed with IRDYE® secondary antibodies, including IRDYE® 680RD goat anti-rabbit antibody, and IRDYE® 800CW goat anti-mouse antibody. The signals are detected using an ODYSSEY® Imaging System.

### Example B2

### Cytokine Modulation Assays

Frozen primary blood mononuclear cells (PBMCs) or frozen CD14+ mobilized peripheral blood monocytes are quick thawed, washed once with RPMI 1640 media supplemented with 10% fetal bovine serum, streptomycin, and penicillin, and plated in 96 well plates at 200,000 cells per well. The cells are pretreated with a compound or DMSO (control) for 1 h and then induced with 100 ng/mL lipopolysaccharide (LPS) for 18-24 h. The supernatant is analyzed for IL-1β, IL-6, and TNFα using Meso Scale assays. The negative control wells are treated with DMSO.

For the IL-2 analysis, a 96 well plate is precoated with 1 μg/mL anti-human CD3 antibody. After washed with PBS, a compound or DMSO (control) is added (50 μL/well), followed by addition of PBMCs diluted at 3-4 million cells/mL (150 μL/well). The plate is incubated for 24 h and the supernatants are collected for IL-2 analysis. IL-2 activity is measured as fold difference from the DMSO control.

### Example B3

### Cell Viability Assays

MOLM-13 cells were cultured in RPMI 1640 media supplemented with 10% fetal bovine serum, streptomycin, and penicillin. The cells were plated in white walled 96-well plates at 2,500 cells/well. The cells were incubated in DMSO (control) or an indicated compound for 3 days at 37° C. and 5% CO₂. Following the incubation period, 100 μL of CELLTITER-GLO® (CTG) reagent was added to each well. Following a 10-min incubation with shaking, luminescence was measured using an ENVISION® Multimode plate reader. Antiproliferative activity of compounds in the MOLM-13 cell viability assay is summarized in Table 3, wherein A represents a value no less than 80%, B represents a value less than 80% but no less than 50%, C represents a value less than 50% but no less than 25%, and D represents a value less than 25% inhibition at a concentration of 1 μM.

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A compound of Formula (I): 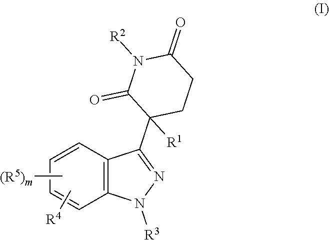
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R¹ is hydrogen, deuterium, C₁₋₆ alkyl, or C₁₋₆ heteroalkyl;
R² is hydrogen or C₁₋₆ alkyl;
R³ and R⁴ are:
(i) R³ is 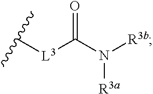
and
R⁴ is hydrogen; or
(ii) R³ is hydrogen, C₁₋₆ alkyl, or C₁₋₆ heteroalkyl; and
R⁴ is 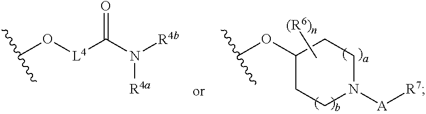
each R⁵ and R⁶ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1c}R^{1c}, —OR^{1a}, —OC(O)R^{1a}, —OC(O)OR^{1a}, —OC(O)NR^{1c}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1b}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR^{1d}, —NR^{1a}C(S)NR^{1b}R^{1c}, —NR^{1a} S(O)R^{1b}, —NR^{1a} S(O)₂R^{1b}, —NR^{1a} S(O)NR^{1b}R^{1c}, —NR^{1a} S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁷ is (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR^{1d}, —NR^{1a}C(S)NR^{1b}R^{1c}, —NR^{1a} S(O)R^{1b}, —NR^{1a} S(O)₂R^{1b}, —NR^{1a} S(O)NR^{1b}R^{1c}, or —NR^{1a} S(O)₂NR^{1b}R^{1c},
R^{3a}, R^{3b}, R^{4a}, and R^{4b} are each independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c}; or R^{3a} and R^{3b} together with the N atom to which they are attached form heterocyclyl; or R^{4a} and R^{4b} together with the N atom to which they are attached form heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
A is —C(O)—, —C(O)C(O)—, —S(O)—, or —S(O₂)—;
L³ and L⁴ are each independently C₁₋₆ alkylene, C₁₋₆ heteroalkylene, C₃₋₁₀ cycloalkylene, or heterocyclylene;
a and b are each independently an integer of 0, 1, 2, or 3;
m is an integer of 0, 1, 2, or 3; and
n is an integer of 0, 1, 2, 3, 4, 5, 6, 7, or 8;
wherein each alkyl, alkylene, heteroalkyl, heteroalkylene, alkenyl, alkynyl, cycloalkyl, cycloalkylene, aryl, aralkyl, heteroaryl, heterocyclyl, and heterocyclylene is optionally substituted with one or more substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NRDR^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR″, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR₄)NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{e}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q^{a}; or (iii) R^{b} and R^{c} together with the N atom to which they are attached form heterocyclyl, optionally substituted with one or more, in one embodiment, one, two, three, of four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. (canceled)

3. The compound of claim 1, having the structure of Formula (IV): 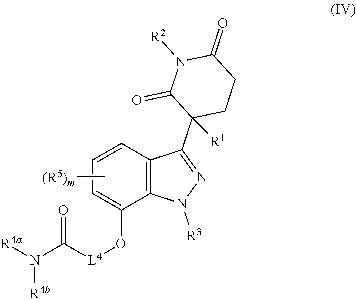
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

4. The compound of claim 1, having the structure of Formula (V): 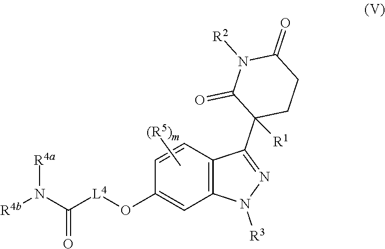
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

5-6. (canceled)

7. The compound of claim 1, having the structure of Formula (VI): 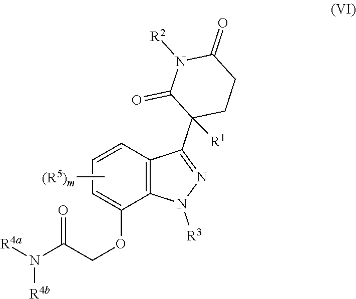
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

8. The compound of claim 1, having the structure of Formula (VII): 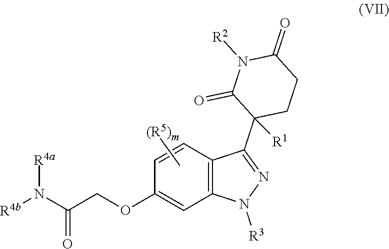
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

9-15. (canceled)

16. The compound of claim 1, having the structure of Formula (VIII): 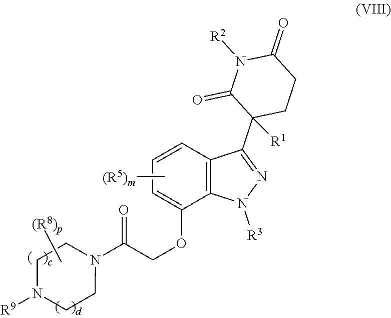
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof: wherein:
each R^{g} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1b}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a} S(O)R^{1b}, —NR^{1a} S(O)₂R^{1d}, —NR^{1a} S(O)NR^{1b}R^{1c}, —NR^{1a} S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁹ is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
c and d are each independently an integer of 0, 1, 2, or 3; and
p is an integer of 0, 1, 2, 3, 4, 5, 6, 7, or 8.

17. The compound of claim 1, having the structure of Formula (IX): 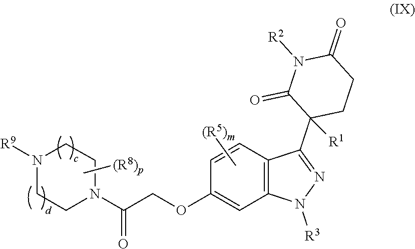
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof: wherein:
each R^{g} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, OS(O)₂R^{1a}, —OS(O)NR^{1b}R^{1c}, —OS(O)₂NR^{1b}R^{1c}, —NR^{1b}R¹⁸, —NR^{1a}C(O)R^{1b}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1b}, —NR^{1a}C(S)OR14, —NR^{1a}C(S)NR^{1b}R^{1c}, —NR^{1a} S(O)R^{1b}, —NR^{1a} S(O)₂R^{1b}, —NR^{1a} S(O)NR^{1b}R^{1c}, —NR^{1a} S(O)₂NR^{1b}R^{1c}, —SR^{1a}, —S(O)R^{1a}, —S(O)₂R^{1a}, —S(O)NR^{1b}R^{1c}, or —S(O)₂NR^{1c}R^{1c};
R⁹ is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1c}R^{1c}, or —S(O)₂NR^{1b}R^{1c};
c and d are each independently an integer of 0, 1, 2, or 3; and
p is an integer of 0, 1, 2, 3, 4, 5, 6, 7, or 8.

18-19. (canceled)

20. The compound of claim 1, having the structure of Formula (X): 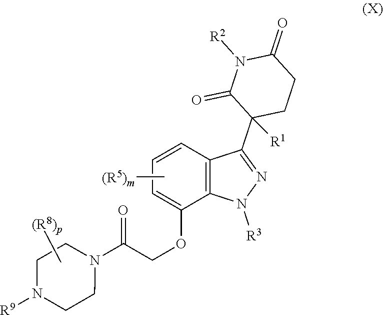
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

21. The compound of claim 1, having the structure of Formula (XIII): 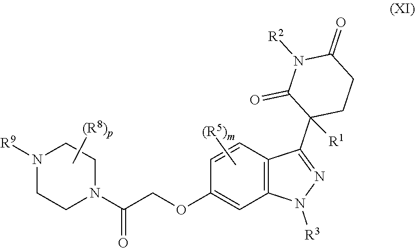
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

22-26. (canceled)

27. The compound of claim 1, having the structure of Formula (XII): 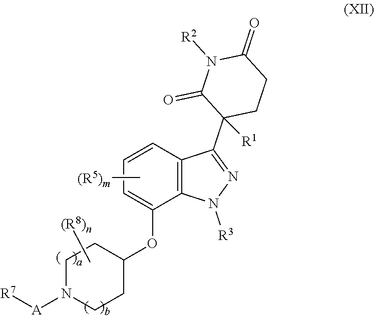
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

28. The compound of claim 1, having the structure of Formula (XIII): 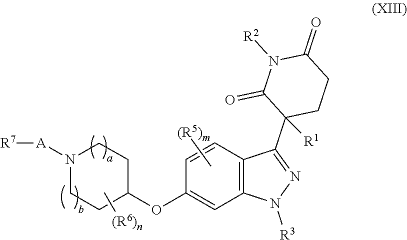
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

29-30. (canceled)

31. The compound of claim 1, having the structure of Formula (XIV): 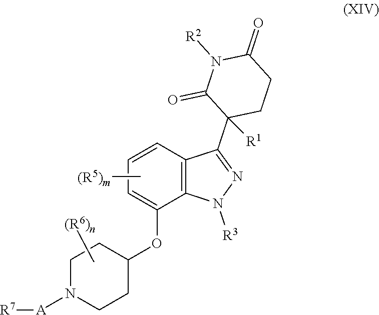
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

32. The compound of claim 1, having the structure of Formula (XV): 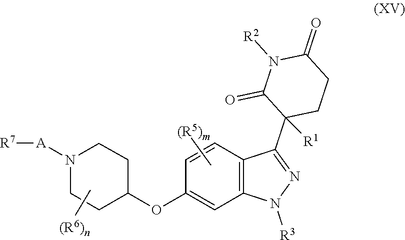
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

33-43. (canceled)

44. The compound of claim 1, having the structure of Formula (XX): 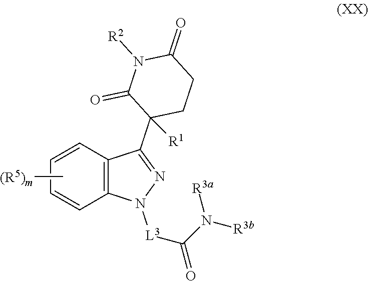
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

45-46. (canceled)

47. The compound of claim 1, having the structure of Formula (XXI): 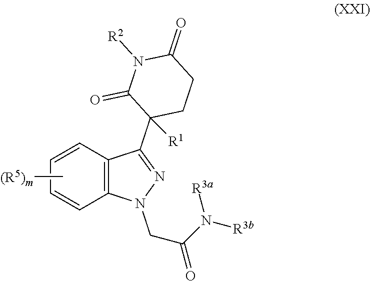
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

48-63. (canceled)

64. The compound of claim 1, wherein the compound is a molecular glue.

65. The compound of claim 1, wherein the compound has a molecular weight of no greater than about 600 Da.

66. The compound of claim 1, wherein the compound is:
2-((3-(2,6-dioxopiperidin-3-yl)-1-methyl-1H-indazol-6-yl)oxy)-N-(oxazol-2-yl)-acetamide A001;
2-((3-(2,6-dioxopiperidin-3-yl)-1-methyl-1H-indazol-6-yl)oxy)-N-((1s,4s)-4-hydroxycyclohexyl)acetamide A002;
2-((3-(2,6-dioxopiperidin-3-yl)-1-methyl-1H-indazol-6-yl)oxy)-N-(3-fluoro-5-morpholinophenyl)acetamide A003;
N-(4-cyano-3-methylisoxazol-5-yl)-2-((3-(2,6-dioxopiperidin-3-yl)-1-methyl-1H-indazol-6-yl)oxy)acetamide A004;
2-((3-(2,6-dioxopiperidin-3-yl)-1-methyl-1H-indazol-6-yl)oxy)-N-((1-methyl-1H-imidazol-5-yl)methyl)acetamide A005;
N-(1-benzylpyrrolidin-3-yl)-2-((3-(2,6-dioxopiperidin-3-yl)-1-methyl-1H-indazol-6-yl)oxy)acetamide A006;
N-(3-cyclopropyl-1-methyl-1H-pyrazol-5-yl)-2-((3-(2,6-dioxopiperidin-3-yl)-1-methyl-1H-indazol-6-yl)oxy)acetamide A007;
2-((3-(2,6-dioxopiperidin-3-yl)-1-methyl-1H-indazol-6-yl)oxy)-N-(2-sulfamoyl-ethyl)acetamide A008;
2-((3-(2,6-dioxopiperidin-3-yl)-1-methyl-1H-indazol-6-yl)oxy)-N-((1-hydroxy-cyclopropyl)methyl)acetamide A009;
2-((3-(2,6-dioxopiperidin-3-yl)-1-methyl-1H-indazol-6-yl)oxy)-N-(1-methyl-1H-pyrazol-4-yl)acetamide A010;
2-((3-(2,6-dioxopiperidin-3-yl)-1-methyl-1H-indazol-6-yl)oxy)-N-((1-methyl-1H-pyrazol-3-yl)methyl)acetamide A011;
N-(2-cyanoethyl)-2-((3-(2,6-dioxopiperidin-3-yl)-1-methyl-1H-indazol-6-yl)oxy)-acetamide A012;
2-((3-(2,6-dioxopiperidin-3-yl)-1-methyl-1H-indazol-6-yl)oxy)-N-(2-(p-tolyloxy)-ethyl)acetamide A013;
N-((4-benzylmorpholin-2-yl)methyl)-2-((3-(2,6-dioxopiperidin-3-yl)-1-methyl-1H-indazol-6-yl)oxy)acetamide A014;
2-((3-(2,6-dioxopiperidin-3-yl)-1-methyl-1H-indazol-6-yl)oxy)-N-(1-phenyl-1H-pyrazol-4-yl)acetamide A015;
2-((3-(2,6-dioxopiperidin-3-yl)-1-methyl-1H-indazol-6-yl)oxy)-N-(thiazol-2-yl-methyl)acetamide A016;
2-((3-(2,6-dioxopiperidin-3-yl)-1-methyl-1H-indazol-6-yl)oxy)-N-(4-((pyrrolidin-1-ylsulfonyl)methyl)phenyl)acetamide A017;
N-(3,5-dimethylisoxazol-4-yl)-2-((3-(2,6-dioxopiperidin-3-yl)-1-methyl-1H-indazol-6-yl)oxy)acetamide A018;
2-((3-(2,6-dioxopiperidin-3-yl)-1-methyl-1H-indazol-6-yl)oxy)-N-(2-(methyl-amino)-2-oxoethyl)acetamide A019;
2-((3-(2,6-dioxopiperidin-3-yl)-1-methyl-1H-indazol-6-yl)oxy)-N-(4-(N-methyl-sulfamoyl)phenyl)acetamide A020;
2-((3-(2,6-dioxopiperidin-3-yl)-1-methyl-1H-indazol-6-yl)oxy)-N-(1-methyl-1H-1,2,4-triazol-5-yl)acetamide A021;
2-((3-(2,6-dioxopiperidin-3-yl)-1-methyl-1H-indazol-6-yl)oxy)-N-(4-(methyl-sulfonyl)phenyl)acetamide A022;
2-((3-(2,6-dioxopiperidin-3-yl)-1-methyl-1H-indazol-6-yl)oxy)-N-(4-sulfamoyl-phenethyl)acetamide A023;
2-((3-(2,6-dioxopiperidin-3-yl)-1-methyl-1H-indazol-6-yl)oxy)-N-(4-(1-methyl-piperidin-4-yl)phenyl)acetamide A024;
2-((3-(2,6-dioxopiperidin-3-yl)-1-methyl-1H-indazol-6-yl)oxy)-N-((6-ethoxy-pyridin-3-yl)methyl)acetamide A025;
2-((3-(2,6-dioxopiperidin-3-yl)-1-methyl-1H-indazol-6-yl)oxy)-N-(pyridazin-3-yl)acetamide A026;
2-((3-(2,6-dioxopiperidin-3-yl)-1-methyl-1H-indazol-6-yl)oxy)-N-((4-hydroxy-tetrahydro-2H-pyran-4-yl)methyl)acetamide A027;
2-((3-(2,6-dioxopiperidin-3-yl)-1-methyl-1H-indazol-6-yl)oxy)-N-(pyrimidin-5-yl)acetamide A028;
2-((3-(2,6-dioxopiperidin-3-yl)-1-methyl-1H-indazol-6-yl)oxy)-N-(1-methyl-azetidin-3-yl)acetamide A029;
2-((3-(2,6-dioxopiperidin-3-yl)-1-methyl-1H-indazol-6-yl)oxy)-N-(3-methyl-isoxazol-5-yl)acetamide A030;
N-(4-(tert-butyl) thiazol-2-yl)-2-((3-(2,6-dioxopiperidin-3-yl)-1-methyl-1H-indazol-6-yl)oxy)acetamide A031;
2-((3-(2,6-dioxopiperidin-3-yl)-1-methyl-1H-indazol-6-yl)oxy)-N-(2-methyl-thiazol-5-yl)acetamide A032;
2-((3-(2,6-dioxopiperidin-3-yl)-1-methyl-1H-indazol-6-yl)oxy)-N-(3-ethyl-isoxazol-5-yl)acetamide A0033;
2-((3-(2,6-dioxopiperidin-3-yl)-1-methyl-1H-indazol-6-yl)oxy)-N-(3-methyl-isothiazol-5-yl)acetamide A0034;
2-((3-(2,6-dioxopiperidin-3-yl)-1-methyl-1H-indazol-6-yl)oxy)-N-(thiazol-4-yl)-acetamide A035;
2-((3-(2,6-dioxopiperidin-3-yl)-1-methyl-1H-indazol-6-yl)oxy)-N-(3-methyl-isothiazol-4-yl)acetamide A036;
N-((1,5-dimethyl-1H-pyrazol-3-yl)methyl)-2-((3-(2,6-dioxopiperidin-3-yl)-1-methyl-1H-indazol-6-yl)oxy)acetamide A037;
N-(3-(1,1-dioxidothiomorpholino) propyl)-2-((3-(2,6-dioxopiperidin-3-yl)-1-methyl-1H-indazol-6-yl)oxy)acetamide A038;
2-((3-(2,6-dioxopiperidin-3-yl)-1-methyl-1H-indazol-6-yl)oxy)-N-(tetrahydro-furan-3-yl)acetamide A039;
2-((3-(2,6-dioxopiperidin-3-yl)-1-methyl-1H-indazol-6-yl)oxy)-N—((R)-1-(pyridin-2-yl)ethyl)acetamide A040;
2-((3-(2,6-dioxopiperidin-3-yl)-1-methyl-1H-indazol-6-yl)oxy)-N-(oxetan-3-yl)-acetamide A041;
2-((3-(2,6-dioxopiperidin-3-yl)-1-methyl-1H-indazol-6-yl)oxy)-N-(4-hydroxy-benzyl)acetamide A042;
2-((3-(2,6-dioxopiperidin-3-yl)-1-methyl-1H-indazol-6-yl)oxy)-N-((2-methyl-1H-indol-5-yl)methyl)acetamide A043;
N-(4-cyanobicyclo[2.2.2]octan-1-yl)-2-((3-(2,6-dioxopiperidin-3-yl)-1-methyl-1H-indazol-6-yl)oxy)acetamide A044;
2-((3-(2,6-dioxopiperidin-3-yl)-1-methyl-1H-indazol-6-yl)oxy)-N-(4-hydroxy-phenethyl)acetamide A045;
N-((3-cyclohexyl-1,2,4-oxadiazol-5-yl)methyl)-2-((3-(2,6-dioxopiperidin-3-yl)-1-methyl-1H-indazol-6-yl)oxy)acetamide A046;
N-(3,5-dimethoxybenzyl)-2-((3-(2,6-dioxopiperidin-3-yl)-1-methyl-1H-indazol-6-yl)oxy)acetamide A047;
2-((3-(2,6-dioxopiperidin-3-yl)-1-methyl-1H-indazol-6-yl)oxy)-N-(4-isopropyl-benzyl)acetamide A048;
2-((3-(2,6-dioxopiperidin-3-yl)-1-methyl-1H-indazol-6-yl)oxy)-N-((3-hydroxy-cyclopentyl)methyl)acetamide A049;
N-(2,5-difluorobenzyl)-2-((3-(2,6-dioxopiperidin-3-yl)-1-methyl-1H-indazol-6-yl)oxy)acetamide A050;
2-((3-(2,6-dioxopiperidin-3-yl)-1-methyl-1H-indazol-6-yl)oxy)-N-((1-methyl-1H-indol-3-yl)methyl)acetamide A051;
2-((3-(2,6-dioxopiperidin-3-yl)-1-methyl-1H-indazol-6-yl)oxy)-N-neopentyl-acetamide A052;
N-(3,5-dichlorobenzyl)-2-((3-(2,6-dioxopiperidin-3-yl)-1-methyl-1H-indazol-6-yl)oxy)acetamide A053;
N-(1,1-dioxidotetrahydro-2H-thiopyran-4-yl)-2-((3-(2,6-dioxopiperidin-3-yl)-1-methyl-1H-indazol-6-yl)oxy)acetamide A054;
2-((3-(2,6-dioxopiperidin-3-yl)-1-methyl-1H-indazol-6-yl)oxy)-N-(4-methyl-tetrahydro-2H-pyran-4-yl)acetamide A055;
N-(2,4-difluorobenzyl)-2-((3-(2,6-dioxopiperidin-3-yl)-1-methyl-1H-indazol-6-yl)oxy)acetamide A056;
N-(2-(benzyloxy)ethyl)-2-((3-(2,6-dioxopiperidin-3-yl)-1-methyl-1H-indazol-6-yl)oxy)acetamide A057;
N-(1,1-dioxidothietan-3-yl)-2-((3-(2,6-dioxopiperidin-3-yl)-1-methyl-1H-indazol-6-yl)oxy)acetamide A058;
2-((3-(2,6-dioxopiperidin-3-yl)-1-methyl-1H-indazol-6-yl)oxy)-N-(1-methyl-piperidin-4-yl)acetamide A059;
N-(3-cyanobenzyl)-2-((3-(2,6-dioxopiperidin-3-yl)-1-methyl-1H-indazol-6-yl)-oxy)acetamide A060;
2-((3-(2,6-dioxopiperidin-3-yl)-1-methyl-1H-indazol-6-yl)oxy)-N-(3-hydroxy-cyclopentyl)acetamide A061;
N-(2,3-dimethyltetrahydrofuran-3-yl)-2-((3-(2,6-dioxopiperidin-3-yl)-1-methyl-1H-indazol-6-yl)oxy)acetamide A062;
N-((2-chlorothiazol-5-yl)methyl)-2-((3-(2,6-dioxopiperidin-3-yl)-1-methyl-1H-indazol-6-yl)oxy)acetamide A063;
2-((3-(2,6-dioxopiperidin-3-yl)-1-methyl-1H-indazol-6-yl)oxy)-N—((R)-1-(5-methylfuran-2-yl) propyl)acetamide A064;
2-((3-(2,6-dioxopiperidin-3-yl)-1-methyl-1H-indazol-6-yl)oxy)-N-(pyridin-2-yl)-acetamide A065;
2-((3-(2,6-dioxopiperidin-3-yl)-1-methyl-1H-indazol-6-yl)oxy)-N—((R)-2-oxo-pyrrolidin-3-yl)acetamide A066;
2-((3-(2,6-dioxopiperidin-3-yl)-1-methyl-1H-indazol-6-yl)oxy)-N-((5-phenyl-1,2,4-oxadiazol-3-yl)methyl)acetamide A067;
N-(cyclopentylmethyl)-2-((3-(2,6-dioxopiperidin-3-yl)-1-methyl-1H-indazol-6-yl)oxy)acetamide A068;
N-(2-(cyclopentyloxy)ethyl)-2-((3-(2,6-dioxopiperidin-3-yl)-1-methyl-1H-indazol-6-yl)oxy)acetamide A069;
N-cyclopropyl-2-((3-(2,6-dioxopiperidin-3-yl)-1-methyl-1H-indazol-6-yl)oxy)-acetamide A070;
N-(2-(3,5-dimethyl-1H-1,2,4-triazol-1-yl)ethyl)-2-((3-(2,6-dioxopiperidin-3-yl)-1-methyl-1H-indazol-6-yl)oxy)acetamide A071;
2-((3-(2,6-dioxopiperidin-3-yl)-1-methyl-1H-indazol-6-yl)oxy)-N-(tetrahydro-2H-pyran-4-yl)acetamide A072;
N-cyclobutyl-2-((3-(2,6-dioxopiperidin-3-yl)-1-methyl-1H-indazol-6-yl)oxy)-acetamide A073;
N—((S)-1-(4-chlorophenyl)ethyl)-2-((3-(2,6-dioxopiperidin-3-yl)-1-methyl-1H-indazol-6-yl)oxy)acetamide A074;
2-((3-(2,6-dioxopiperidin-3-yl)-1-methyl-1H-indazol-6-yl)oxy)-N-(1-isopropyl-1H-pyrazol-4-yl)acetamide A075;
2-((3-(2,6-dioxopiperidin-3-yl)-1-methyl-1H-indazol-6-yl)oxy)-N-(1-(tetrahydro-2H-pyran-4-yl)-1H-pyrazol-4-yl)acetamide A076;
2-((3-(2,6-dioxopiperidin-3-yl)-1-methyl-1H-indazol-6-yl)oxy)-N-((1-methyl-2-oxo-1,2-dihydropyridin-4-yl)methyl)acetamide A077;
2-((3-(2,6-dioxopiperidin-3-yl)-1-methyl-1H-indazol-6-yl)oxy)-N-(4-(2-methyl-1H-imidazol-1-yl)phenyl)acetamide A078;
2-((3-(2,6-dioxopiperidin-3-yl)-1-methyl-1H-indazol-6-yl)oxy)-N-(3-fluoro-bicyclo[1.1.1]pentan-1-yl)acetamide A079;
N-(4-(1H-imidazol-2-yl)phenyl)-2-((3-(2,6-dioxopiperidin-3-yl)-1-methyl-1H-indazol-6-yl)oxy)acetamide A080;
2-((3-(2,6-dioxopiperidin-3-yl)-1-methyl-1H-indazol-6-yl)oxy)-N-(4-(4-methyl-piperazin-1-yl)phenyl)acetamide A081;
2-((3-(2,6-dioxopiperidin-3-yl)-1-methyl-1H-indazol-6-yl)oxy)-N-(4-(oxazol-5-yl)phenyl)acetamide A082;
2-((3-(2,6-dioxopiperidin-3-yl)-1-methyl-1H-indazol-6-yl)oxy)-N-(1-methyl-1H-pyrazol-5-yl)acetamide A083;
2-((3-(2,6-dioxopiperidin-3-yl)-1-methyl-1H-indazol-6-yl)oxy)-N-(4-(p-tolyloxy)-phenyl)acetamide A084;
N-(3-(tert-butyl)phenyl)-2-((3-(2,6-dioxopiperidin-3-yl)-1-methyl-1H-indazol-6-yl)oxy)acetamide A085;
2-((3-(2,6-dioxopiperidin-3-yl)-1-methyl-1H-indazol-6-yl)oxy)-N-(1-(oxetan-3-yl)-1H-pyrazol-4-yl)acetamide A086;
N-(4-(1H-imidazol-5-yl)phenyl)-2-((3-(2,6-dioxopiperidin-3-yl)-1-methyl-1H-indazol-6-yl)oxy)acetamide A087;
2-((3-(2,6-dioxopiperidin-3-yl)-1-methyl-1H-indazol-6-yl)oxy)-N-(4-(trifluoro-methoxy)phenyl)acetamide A088;
2-((3-(2,6-dioxopiperidin-3-yl)-1-methyl-1H-indazol-6-yl)oxy)-N-(3-isopropoxy-phenyl)acetamide A089;
N-(5-cyclopropyl-1H-pyrazol-3-yl)-2-((3-(2,6-dioxopiperidin-3-yl)-1-methyl-1H-indazol-6-yl)oxy)acetamide A090;
N-(3-(1H-imidazol-1-yl)phenyl)-2-((3-(2,6-dioxopiperidin-3-yl)-1-methyl-1H-indazol-6-yl)oxy)acetamide A091;
N-(cyclopropylmethyl)-2-((3-(2,6-dioxopiperidin-3-yl)-1-methyl-1H-indazol-6-yl)oxy)acetamide A092;
N-cyclohexyl-2-((3-(2,6-dioxopiperidin-3-yl)-1-methyl-1H-indazol-6-yl)oxy)-acetamide A093;
N-cyclopentyl-2-((3-(2,6-dioxopiperidin-3-yl)-1-methyl-1H-indazol-6-yl)oxy)-acetamide A094;
2-((3-(2,6-dioxopiperidin-3-yl)-1-methyl-1H-indazol-6-yl)oxy)-N-(thiophen-2-ylmethyl)acetamide A095;
2-((3-(2,6-dioxopiperidin-3-yl)-1-methyl-1H-indazol-6-yl)oxy)-N-(4-(morpholinomethyl)benzyl)acetamide A096;
N-(3-cyano-4,5-dimethyl-1H-pyrrol-2-yl)-2-((3-(2,6-dioxopiperidin-3-yl)-1-methyl-1H-indazol-7-yl)oxy)acetamide B001;
2-((3-(2,6-dioxopiperidin-3-yl)-1-methyl-1H-indazol-7-yl)oxy)-N-(4-(trifluoro-methoxy)phenyl)acetamide B002;
6-(tert-butyl)₃-ethyl 2-(2-((3-(2,6-dioxopiperidin-3-yl)-1-methyl-1H-indazol-7-yl)oxy)acetamido)-4,7-dihydrothieno[2,3-c]pyridine-3,6 (5H)-dicarboxylate B003;
diethyl 3-(2-((3-(2,6-dioxopiperidin-3-yl)-1-methyl-1H-indazol-7-yl)oxy)-acetamido)-1H-pyrrole-2,4-dicarboxylate B004;
ethyl 2-(2-((3-(2,6-dioxopiperidin-3-yl)-1-methyl-1H-indazol-7-yl)oxy)-acetamido)-4-methyl-4,5,6,7-tetrahydrobenzo[b]thiophene-3-carboxylate B005;
N-(3-cyano-1H-pyrrol-2-yl)-2-((3-(2,6-dioxopiperidin-3-yl)-1-methyl-1H-indazol-7-yl)oxy)acetamide B006;
ethyl 5-(2-((3-(2,6-dioxopiperidin-3-yl)-1-methyl-1H-indazol-7-yl)oxy)-acetamido)-3-methyl-1H-pyrazole-4-carboxylate B007;
2-((3-(2,6-dioxopiperidin-3-yl)-1-methyl-1H-indazol-7-yl)oxy)-N-(3-(pyridin-2-yl)-1H-pyrazol-4-yl)acetamide B008;
diethyl 2-(2-((3-(2,6-dioxopiperidin-3-yl)-1-methyl-1H-indazol-7-yl)oxy)-acetamido)thiophene-3,4-dicarboxylate B009;
N-(5-chloro-2-cyanophenyl)-2-((3-(2,6-dioxopiperidin-3-yl)-1-methyl-1H-indazol-7-yl)oxy)acetamide B010;
2-((3-(2,6-dioxopiperidin-3-yl)-1-methyl-1H-indazol-7-yl)oxy)-N-(4-(oxazol-5-yl)phenyl)acetamide B011;
N-(4-cyano-3-methylisothiazol-5-yl)-2-((3-(2,6-dioxopiperidin-3-yl)-1-methyl-1H-indazol-7-yl)oxy)acetamide B012;
N-(2-cyano-3-methoxyphenyl)-2-((3-(2,6-dioxopiperidin-3-yl)-1-methyl-1H-indazol-7-yl)oxy)acetamide B013;
N-(4-cyano-1-phenyl-1H-pyrazol-5-yl)-2-((3-(2,6-dioxopiperidin-3-yl)-1-methyl-1H-indazol-7-yl)oxy)acetamide B014;
N-(3-cyano-6-methyl-4,5,6,7-tetrahydrobenzo[b]thiophen-2-yl)-2-((3-(2,6-dioxo-piperidin-3-yl)-1-methyl-1H-indazol-7-yl)oxy)acetamide B015;
N-(2-cyano-5-(trifluoromethyl)phenyl)-2-((3-(2,6-dioxopiperidin-3-yl)-1-methyl-1H-indazol-7-yl)oxy)acetamide B016;
N-(2-cyano-3-methylphenyl)-2-((3-(2,6-dioxopiperidin-3-yl)-1-methyl-1H-indazol-7-yl)oxy)acetamide B017;
N-(2-cyano-4-methoxyphenyl)-2-((3-(2,6-dioxopiperidin-3-yl)-1-methyl-1H-indazol-7-yl)oxy)acetamide B018;
methyl 4-(2-((3-(2,6-dioxopiperidin-3-yl)-1-methyl-1H-indazol-7-yl)oxy)-acetamido)-2-methylthiophene-3-carboxylate B019;
ethyl 5-(2-((3-(2,6-dioxopiperidin-3-yl)-1-methyl-1H-indazol-7-yl)oxy)-acetamido) thiazole-4-carboxylate B020;
ethyl 2-(2-((3-(2,6-dioxopiperidin-3-yl)-1-methyl-1H-indazol-7-yl)oxy)-acetamido)-4-isopropylthiophene-3-carboxylate B021;
N-(3-cyano-4-methylthiophen-2-yl)-2-((3-(2,6-dioxopiperidin-3-yl)-1-methyl-1H-indazol-7-yl)oxy)acetamide B022;
N-(3-(2-chlorobenzoyl)-5-ethylthiophen-2-yl)-2-((3-(2,6-dioxopiperidin-3-yl)-1-methyl-1H-indazol-7-yl)oxy)acetamide B023;
N-(2-cyano-6-methylphenyl)-2-((3-(2,6-dioxopiperidin-3-yl)-1-methyl-1H-indazol-7-yl)oxy)acetamide B024;
N-(3-cyano-4-methyl-1H-pyrrol-2-yl)-2-((3-(2,6-dioxopiperidin-3-yl)-1-methyl-1H-indazol-7-yl)oxy)acetamide B025;
ethyl 5-cyano-4-(2-((3-(2,6-dioxopiperidin-3-yl)-1-methyl-1H-indazol-7-yl)oxy)-acetamido)-2-(methylthio)thiophene-3-carboxylate B026;
N-(5-(tert-butyl)-1,3,4-thiadiazol-2-yl)-2-((3-(2,6-dioxopiperidin-3-yl)-1-methyl-1H-indazol-7-yl)oxy)acetamide B027;
ethyl 2-(2-((3-(2,6-dioxopiperidin-3-yl)-1-methyl-1H-indazol-7-yl)oxy)-acetamido)-4-phenylthiophene-3-carboxylate B028;
methyl 2-(2-((3-(2,6-dioxopiperidin-3-yl)-1-methyl-1H-indazol-7-yl)oxy)-acetamido)-1H-indole-3-carboxylate B029;
2-((3-(2,6-dioxopiperidin-3-yl)-1-methyl-1H-indazol-7-yl)oxy)-N-(2-hydroxy-2,3-dihydro-1H-inden-1-yl)acetamide B030;
N-(4-cyano-3-methyl-1-phenyl-1H-pyrazol-5-yl)-2-((3-(2,6-dioxopiperidin-3-yl)-1-methyl-1H-indazol-7-yl)oxy)acetamide B031;
3-cyano-4-(2-((3-(2,6-dioxopiperidin-3-yl)-1-methyl-1H-indazol-7-yl)oxy)-acetamido)benzoic acid B032;
2-((3-(2,6-dioxopiperidin-3-yl)-1-methyl-1H-indazol-7-yl)oxy)-N-(4-(trifluoro-methyl) thiazol-2-yl)acetamide B033;
N-(3-bromo-2-cyanophenyl)-2-((3-(2,6-dioxopiperidin-3-yl)-1-methyl-1H-indazol-7-yl)oxy)acetamide B034;
2-((3-(2,6-dioxopiperidin-3-yl)-1-methyl-1H-indazol-7-yl)oxy)-N-(thiazol-4-yl-methyl)acetamide B035;
methyl 5-(2-((3-(2,6-dioxopiperidin-3-yl)-1-methyl-1H-indazol-7-yl)oxy)-acetamido)-3-methylthiophene-2-carboxylate B036;
N-(3-cyano-5-(trifluoromethyl)phenyl)-2-((3-(2,6-dioxopiperidin-3-yl)-1-methyl-1H-indazol-7-yl)oxy)acetamide B037;
2-((3-(2,6-dioxopiperidin-3-yl)-1-methyl-1H-indazol-7-yl)oxy)-N-(3-methyl-isothiazol-5-yl)acetamide B038;
methyl 2-(2-((3-(2,6-dioxopiperidin-3-yl)-1-methyl-1H-indazol-7-yl)oxy)-acetamido)-4,5-dimethylthiophene-3-carboxylate B039;
N-(2-cyanopyridin-3-yl)-2-((3-(2,6-dioxopiperidin-3-yl)-1-methyl-1H-indazol-7-yl)oxy)acetamide B040;
2-((3-(2,6-dioxopiperidin-3-yl)-1-methyl-1H-indazol-7-yl)oxy)-N-(3-phenyl-1H-pyrazol-4-yl)acetamide B041;
N-(3-cyano-4,5-dimethylthiophen-2-yl)-2-((3-(2,6-dioxopiperidin-3-yl)-1-methyl-1H-indazol-7-yl)oxy)acetamide B042;
methyl 2-(2-((3-(2,6-dioxopiperidin-3-yl)-1-methyl-1H-indazol-7-yl)oxy)-acetamido)-4,5,6,7-tetrahydrobenzo[b]thiophene-3-carboxylate B043;
5-cyano-2-(2-((3-(2,6-dioxopiperidin-3-yl)-1-methyl-1H-indazol-7-yl)oxy)-acetamido)benzoic acid B044;
methyl 3-(2-((3-(2,6-dioxopiperidin-3-yl)-1-methyl-1H-indazol-7-yl)oxy)-acetamido)-5-iodothiophene-2-carboxylate B045;
N-(2-cyano-3-fluorophenyl)-2-((3-(2,6-dioxopiperidin-3-yl)-1-methyl-1H-indazol-7-yl)oxy)acetamide B046;
2-((3-(2,6-dioxopiperidin-3-yl)-1-methyl-1H-indazol-7-yl)oxy)-N-(4-methyl-1,2,5-oxadiazol-3-yl)acetamide B047;
N-(2-cyano-4-fluorophenyl)-2-((3-(2,6-dioxopiperidin-3-yl)-1-methyl-1H-indazol-7-yl)oxy)acetamide B048;
N-(2-cyano-5-methylpyridin-3-yl)-2-((3-(2,6-dioxopiperidin-3-yl)-1-methyl-1H-indazol-7-yl)oxy)acetamide B049;
N-(2-cyanothiophen-3-yl)-2-((3-(2,6-dioxopiperidin-3-yl)-1-methyl-1H-indazol-7-yl)oxy)acetamide B050;
N-(2-cyano-4,6-difluorophenyl)-2-((3-(2,6-dioxopiperidin-3-yl)-1-methyl-1H-indazol-7-yl)oxy)acetamide B051;
N-(2-cyano-4-methylphenyl)-2-((3-(2,6-dioxopiperidin-3-yl)-1-methyl-1H-indazol-7-yl)oxy)acetamide B052;
N-(2-cyanobenzyl)-2-((3-(2,6-dioxopiperidin-3-yl)-1-methyl-1H-indazol-7-yl)-oxy)acetamide B053;
N-(4-cyano-1,3-dimethyl-1H-pyrazol-5-yl)-2-((3-(2,6-dioxopiperidin-3-yl)-1-methyl-1H-indazol-7-yl)oxy)acetamide B054;
methyl 3-(2-((3-(2,6-dioxopiperidin-3-yl)-1-methyl-1H-indazol-7-yl)oxy)-acetamido)-2H-thieno[3,2-c]pyrazole-5-carboxylate B055;
N-(2-cyanonaphthalen-1-yl)-2-((3-(2,6-dioxopiperidin-3-yl)-1-methyl-1H-indazol-7-yl)oxy)acetamide B056;
N-(6-chloro-3-cyanopyridin-2-yl)-2-((3-(2,6-dioxopiperidin-3-yl)-1-methyl-1H-indazol-7-yl)oxy)acetamide B057;
N-(3-cyanothiophen-2-yl)-2-((3-(2,6-dioxopiperidin-3-yl)-1-methyl-1H-indazol-7-yl)oxy)acetamide B058;
N-(2-cyano-5-methylphenyl)-2-((3-(2,6-dioxopiperidin-3-yl)-1-methyl-1H-indazol-7-yl)oxy)acetamide B059;
2-((3-(2,6-dioxopiperidin-3-yl)-1-methyl-1H-indazol-7-yl)oxy)-N-(1-methyl-3-(trifluoromethyl)-1H-pyrazol-5-yl)acetamide B060;
N-(4-(3-chlorophenyl)-1H-pyrazol-5-yl)-2-((3-(2,6-dioxopiperidin-3-yl)-1-methyl-1H-indazol-7-yl)oxy)acetamide B061;
N-(2-cyano-6-fluorophenyl)-2-((3-(2,6-dioxopiperidin-3-yl)-1-methyl-1H-indazol-7-yl)oxy)acetamide B062;
ethyl 2-(2-((3-(2,6-dioxopiperidin-3-yl)-1-methyl-1H-indazol-7-yl)oxy)-acetamido)-4,5,6,7-tetrahydrobenzo[b]thiophene-3-carboxylate B063;
N-((1-cyanocyclopentyl)methyl)-2-((3-(2,6-dioxopiperidin-3-yl)-1-methyl-1H-indazol-7-yl)oxy)acetamide B064;
N-(4-cyano-1-methyl-1H-pyrazol-5-yl)-2-((3-(2,6-dioxopiperidin-3-yl)-1-methyl-1H-indazol-7-yl)oxy)acetamide B065;
N-(4-cyano-3-methylisoxazol-5-yl)-2-((3-(2,6-dioxopiperidin-3-yl)-1-methyl-1H-indazol-7-yl)oxy)acetamide B066;
N-(1-(tert-butyl)-3-methyl-1H-pyrazol-5-yl)-2-((3-(2,6-dioxopiperidin-3-yl)-1-methyl-1H-indazol-7-yl)oxy)acetamide B067;
2-((3-(2,6-dioxopiperidin-3-yl)-1-methyl-1H-indazol-7-yl)oxy)-N-(5-(furan-2-yl)-1,3,4-oxadiazol-2-yl)acetamide B068;
2-((3-(2,6-dioxopiperidin-3-yl)-1-methyl-1H-indazol-7-yl)oxy)-N-(5-methyl-1,3,4-oxadiazol-2-yl)acetamide B069;
N-(3,5-dimethylisoxazol-4-yl)-2-((3-(2,6-dioxopiperidin-3-yl)-1-methyl-1H-indazol-7-yl)oxy)acetamide B070;
2-((3-(2,6-dioxopiperidin-3-yl)-1-methyl-1H-indazol-7-yl)oxy)-N-((4-hydroxy-tetrahydro-2H-pyran-4-yl)methyl)acetamide B071;
2-((3-(2,6-dioxopiperidin-3-yl)-1-methyl-1H-indazol-7-yl)oxy)-N-(5-ethyl-isoxazol-3-yl)acetamide B072;
2-((3-(2,6-dioxopiperidin-3-yl)-1-methyl-1H-indazol-7-yl)oxy)-N-(3-ethyl-isoxazol-5-yl)acetamide B073;
2-((3-(2,6-dioxopiperidin-3-yl)-1-methyl-1H-indazol-7-yl)oxy)-N-(3-methyl-isoxazol-5-yl)acetamide B074;
2-((3-(2,6-dioxopiperidin-3-yl)-1-methyl-1H-indazol-7-yl)oxy)-N-(isoxazol-5-yl)acetamide B075;
2-((3-(2,6-dioxopiperidin-3-yl)-1-methyl-1H-indazol-7-yl)oxy)-N-(1-methyl-1H-1,2,4-triazol-5-yl)acetamide B076;
2-((3-(2,6-dioxopiperidin-3-yl)-1-methyl-1H-indazol-7-yl)oxy)-N-((8-methyl-[1,2,4]triazolo[4,3-a]pyridin-3-yl)methyl)acetamide B077;
2-((3-(2,6-dioxopiperidin-3-yl)-1-methyl-1H-indazol-7-yl)oxy)-N-(4-(2-methyl-1H-imidazol-1-yl)phenyl)acetamide B078;
2-((3-(2,6-dioxopiperidin-3-yl)-1-methyl-1H-indazol-7-yl)oxy)-N-((6-ethoxy-pyridin-3-yl)methyl)acetamide B079;
2-((3-(2,6-dioxopiperidin-3-yl)-1-methyl-1H-indazol-7-yl)oxy)-N—(((R)-5-oxo-pyrrolidin-2-yl)methyl)acetamide B080;
2-((3-(2,6-dioxopiperidin-3-yl)-1-methyl-1H-indazol-7-yl)oxy)-N-(1-methyl-2-oxopiperidin-4-yl)acetamide B081;
2-((3-(2,6-dioxopiperidin-3-yl)-1-methyl-1H-indazol-7-yl)oxy)-N-(2-(5-isopropyl-1,2,4-oxadiazol-3-yl)ethyl)acetamide B082;
2-((3-(2,6-dioxopiperidin-3-yl)-1-methyl-1H-indazol-7-yl)oxy)-N-(4-(1-methyl-piperidin-4-yl)phenyl)acetamide B083;
2-((3-(2,6-dioxopiperidin-3-yl)-1-methyl-1H-indazol-7-yl)oxy)-N-(4-(N-methyl-sulfamoyl)phenyl)acetamide B084;
2-((3-(2,6-dioxopiperidin-3-yl)-1-methyl-1H-indazol-7-yl)oxy)-N—((R)-2-oxo-azepan-3-yl)acetamide B085;
N-(4-(1H-imidazol-2-yl)phenyl)-2-((3-(2,6-dioxopiperidin-3-yl)-1-methyl-1H-indazol-7-yl)oxy)acetamide B086;
2-((3-(2,6-dioxopiperidin-3-yl)-1-methyl-1H-indazol-7-yl)oxy)-N-(thiazol-4-yl)-acetamide B087;
2-((3-(2,6-dioxopiperidin-3-yl)-1-methyl-1H-indazol-7-yl)oxy)-N-(4-(methyl-sulfonyl)phenyl)acetamide B088;
2-((3-(2,6-dioxopiperidin-3-yl)-1-methyl-1H-indazol-7-yl)oxy)-N-(pyridazin-3-yl)acetamide B089;
2-((3-(2,6-dioxopiperidin-3-yl)-1-methyl-1H-indazol-7-yl)oxy)-N-(4-(4-methyl-piperazin-1-yl)phenyl)acetamide B090;
2-((3-(2,6-dioxopiperidin-3-yl)-1-methyl-1H-indazol-7-yl)oxy)-N—((R)-1-(pyridin-2-yl)ethyl)acetamide B091;
2-((3-(2,6-dioxopiperidin-3-yl)-1-methyl-1H-indazol-7-yl)oxy)-N-(oxazol-2-yl)-acetamide B092;
2-((3-(2,6-dioxopiperidin-3-yl)-1-methyl-1H-indazol-7-yl)oxy)-N-(4-(2-oxo-pyrrolidin-1-yl)benzyl)acetamide B093;
2-((3-(2,6-dioxopiperidin-3-yl)-1-methyl-1H-indazol-7-yl)oxy)-N-(oxetan-3-yl)-acetamide B094;
2-((3-(2,6-dioxopiperidin-3-yl)-1-methyl-1H-indazol-7-yl)oxy)-N-(1-methyl-1H-pyrazol-4-yl)acetamide B095;
2-((3-(2,6-dioxopiperidin-3-yl)-1-methyl-1H-indazol-7-yl)oxy)-N—(((S)-oxetan-2-yl)methyl)acetamide B096;
N-((2-chlorothiazol-5-yl)methyl)-2-((3-(2,6-dioxopiperidin-3-yl)-1-methyl-1H-indazol-7-yl)oxy)acetamide B097;
2-((3-(2,6-dioxopiperidin-3-yl)-1-methyl-1H-indazol-7-yl)oxy)-N—((R)-1-(4-fluorophenyl)ethyl)acetamide B098;
2-((3-(2,6-dioxopiperidin-3-yl)-1-methyl-1H-indazol-7-yl)oxy)-N—((S)-1-phenyl-propyl)acetamide B099;
N—((S)-1-(4-chlorophenyl)ethyl)-2-((3-(2,6-dioxopiperidin-3-yl)-1-methyl-1H-indazol-7-yl)oxy)acetamide B100;
2-((3-(2,6-dioxopiperidin-3-yl)-1-methyl-1H-indazol-7-yl)oxy)-N-(1-phenyl-1H-pyrazol-4-yl)acetamide B101;
2-((3-(2,6-dioxopiperidin-3-yl)-1-methyl-1H-indazol-7-yl)oxy)-N—((S)-1-(p-tolyl)-ethyl)acetamide B102;
N-(3,5-dimethyl-1H-pyrazol-4-yl)-2-((3-(2,6-dioxopiperidin-3-yl)-1-methyl-1H-indazol-7-yl)oxy)acetamide B103;
N-(3-(difluoromethoxy)-1H-pyrazol-5-yl)-2-((3-(2,6-dioxopiperidin-3-yl)-1-methyl-1H-indazol-7-yl)oxy)acetamide B104;
2-((3-(2,6-dioxopiperidin-3-yl)-1-methyl-1H-indazol-7-yl)oxy)-N-(2-(methyl-amino)-2-oxoethyl)acetamide B105;
2-((3-(2,6-dioxopiperidin-3-yl)-1-methyl-1H-indazol-7-yl)oxy)-N-((1s,4s)-4-hydroxycyclohexyl)acetamide B106;
2-((3-(2,6-dioxopiperidin-3-yl)-1-methyl-1H-indazol-7-yl)oxy)-N-(2-oxo-piperidin-3-yl)acetamide B107;
2-((3-(2,6-dioxopiperidin-3-yl)-1-methyl-1H-indazol-7-yl)oxy)-N-((1r,4r)-4-methoxycyclohexyl)acetamide B108;
N-(3-(tert-butyl)-1-phenyl-1H-pyrazol-5-yl)-2-((3-(2,6-dioxopiperidin-3-yl)-1-methyl-1H-indazol-7-yl)oxy)acetamide B109;
N-(4-(1H-pyrrol-1-yl)phenyl)-2-((3-(2,6-dioxopiperidin-3-yl)-1-methyl-1H-indazol-7-yl)oxy)acetamide B110;
2-((3-(2,6-dioxopiperidin-3-yl)-1-methyl-1H-indazol-7-yl)oxy)-N-(4-hydroxy-phenethyl)acetamide B111;
2-((3-(2,6-dioxopiperidin-3-yl)-1-methyl-1H-indazol-7-yl)oxy)-N-((2-methyl-1H-indol-5-yl)methyl)acetamide B112;
2-((3-(2,6-dioxopiperidin-3-yl)-1-methyl-1H-indazol-7-yl)oxy)-N-(2-methyl-thiazol-5-yl)acetamide B113;
2-((3-(2,6-dioxopiperidin-3-yl)-1-methyl-1H-indazol-7-yl)oxy)-N-(pyridin-2-yl)-acetamide B114;
N-(4-(tert-butyl) thiazol-2-yl)-2-((3-(2,6-dioxopiperidin-3-yl)-1-methyl-1H-indazol-7-yl)oxy)acetamide B115;
2-((3-(2,6-dioxopiperidin-3-yl)-1-methyl-1H-indazol-7-yl)oxy)-N-((3-hydroxy-cyclopentyl)methyl)acetamide B116;
2-((3-(2,6-dioxopiperidin-3-yl)-1-methyl-1H-indazol-7-yl)oxy)-N-(3-methyl-isothiazol-4-yl)acetamide B117;
2-((3-(2,6-dioxopiperidin-3-yl)-1-methyl-1H-indazol-7-yl)oxy)-N-(4-((pyrrolidin-1-ylsulfonyl)methyl)phenyl)acetamide B118;
N-(4-(sec-butoxy)benzyl)-2-((3-(2,6-dioxopiperidin-3-yl)-1-methyl-1H-indazol-7-yl)oxy)acetamide B119;
2-((3-(2,6-dioxopiperidin-3-yl)-1-methyl-1H-indazol-7-yl)oxy)-N-(1-methyl-piperidin-4-yl)acetamide B120;
2-((3-(2,6-dioxopiperidin-3-yl)-1-methyl-1H-indazol-7-yl)oxy)-N-(3-hydroxy-cyclopentyl)acetamide B121;
2-((3-(2,6-dioxopiperidin-3-yl)-1-methyl-1H-indazol-7-yl)oxy)-N-(3-fluoro-5-morpholinophenyl)acetamide B122;
N-(2-(3,5-dimethyl-1H-1,2,4-triazol-1-yl)ethyl)-2-((3-(2,6-dioxopiperidin-3-yl)-1-methyl-1H-indazol-7-yl)oxy)acetamide B123;
N-(4-cyano-1-(2-hydroxyethyl)-1H-pyrazol-5-yl)-2-((3-(2,6-dioxopiperidin-3-yl)-1-methyl-1H-indazol-7-yl)oxy)acetamide B124;
2-((3-(2,6-dioxopiperidin-3-yl)-1-methyl-1H-indazol-7-yl)oxy)-N-((1-methyl-1H-imidazol-5-yl)methyl)acetamide B125;
N-(1,1-dioxidotetrahydro-2H-thiopyran-4-yl)-2-((3-(2,6-dioxopiperidin-3-yl)-1-methyl-1H-indazol-7-yl)oxy)acetamide B126;
N-(4-cyanobicyclo[2.2.2]octan-1-yl)-2-((3-(2,6-dioxopiperidin-3-yl)-1-methyl-1H-indazol-7-yl)oxy)acetamide B127;
N-(3-cyanobenzyl)-2-((3-(2,6-dioxopiperidin-3-yl)-1-methyl-1H-indazol-7-yl)-oxy)acetamide B128;
2-((3-(2,6-dioxopiperidin-3-yl)-1-methyl-1H-indazol-7-yl)oxy)-N-((1-methyl-2-oxo-1,2-dihydropyridin-4-yl)methyl)acetamide B129;
N-(3-cyclopropyl-1-methyl-1H-pyrazol-5-yl)-2-((3-(2,6-dioxopiperidin-3-yl)-1-methyl-1H-indazol-7-yl)oxy)acetamide B130;
2-((3-(2,6-dioxopiperidin-3-yl)-1-methyl-1H-indazol-7-yl)oxy)-N-(4-(p-tolyloxy)-phenyl)acetamide B131;
2-((3-(2,6-dioxopiperidin-3-yl)-1-methyl-1H-indazol-7-yl)oxy)-N-((1-hydroxy-cyclopropyl)methyl)acetamide B132;
2-((3-(2,6-dioxopiperidin-3-yl)-1-methyl-1H-indazol-7-yl)oxy)-N-((5-methyl-isoxazol-3-yl)methyl)acetamide B133;
2-((3-(2,6-dioxopiperidin-3-yl)-1-methyl-1H-indazol-7-yl)oxy)-N-((1-methyl-1H-indol-3-yl)methyl)acetamide B134;
2-((3-(2,6-dioxopiperidin-3-yl)-1-methyl-1H-indazol-7-yl)oxy)-N-(1-methyl-pyrrolidin-3-yl)acetamide B135;
N-(2-(benzyloxy)ethyl)-2-((3-(2,6-dioxopiperidin-3-yl)-1-methyl-1H-indazol-7-yl)oxy)acetamide B136;
N-(1-benzylpyrrolidin-3-yl)-2-((3-(2,6-dioxopiperidin-3-yl)-1-methyl-1H-indazol-7-yl)oxy)acetamide B137;
2-((3-(2,6-dioxopiperidin-3-yl)-1-methyl-1H-indazol-7-yl)oxy)-N-(4-isopropyl-benzyl)acetamide B138;
2-((3-(2,6-dioxopiperidin-3-yl)-1-methyl-1H-indazol-7-yl)oxy)-N-(tetrahydro-furan-3-yl)acetamide B139;
2-((3-(2,6-dioxopiperidin-3-yl)-1-methyl-1H-indazol-7-yl)oxy)-N-(1-methyl-azetidin-3-yl)acetamide B140;
2-((3-(2,6-dioxopiperidin-3-yl)-1-methyl-1H-indazol-7-yl)oxy)-N-(1-methyl-1H-pyrazol-5-yl)acetamide B141;
2-((3-(2,6-dioxopiperidin-3-yl)-1-methyl-1H-indazol-7-yl)oxy)-N-((3-methyl-1,2,4-oxadiazol-5-yl)methyl)acetamide B142;
2-((3-(2,6-dioxopiperidin-3-yl)-1-methyl-1H-indazol-7-yl)oxy)-N-(4-sulfamoyl-phenethyl)acetamide B143;
2-((3-(2,6-dioxopiperidin-3-yl)-1-methyl-1H-indazol-7-yl)oxy)-N-((tetrahydro-furan-2-yl)methyl)acetamide B144;
2-((3-(2,6-dioxopiperidin-3-yl)-1-methyl-1H-indazol-7-yl)oxy)-N-((1-methyl-1H-pyrazol-3-yl)methyl)acetamide B145;
2-((3-(2,6-dioxopiperidin-3-yl)-1-methyl-1H-indazol-7-yl)oxy)-N-(1-propyl-piperidin-4-yl)acetamide B146;
N-((3-cyclohexyl-1,2,4-oxadiazol-5-yl)methyl)-2-((3-(2,6-dioxopiperidin-3-yl)-1-methyl-1H-indazol-7-yl)oxy)acetamide B147;
2-((3-(2,6-dioxopiperidin-3-yl)-1-methyl-1H-indazol-7-yl)oxy)-N-((5-phenyl-1,2,4-oxadiazol-3-yl)methyl)acetamide B148;
2-((3-(2,6-dioxopiperidin-3-yl)-1-methyl-1H-indazol-7-yl)oxy)-N-(3-fluoro-bicyclo[1.1.1]pentan-1-yl)acetamide B149;
2-((3-(2,6-dioxopiperidin-3-yl)-1-methyl-1H-indazol-7-yl)oxy)-N-(1-isopropyl-1H-pyrazol-4-yl)acetamide B150;
2-((3-(2,6-dioxopiperidin-3-yl)-1-methyl-1H-indazol-7-yl)oxy)-N-(2-(p-tolyloxy)-ethyl)acetamide B151;
N-(1,1-dioxidothietan-3-yl)-2-((3-(2,6-dioxopiperidin-3-yl)-1-methyl-1H-indazol-7-yl)oxy)acetamide B152;
N-(3,5-dimethoxybenzyl)-2-((3-(2,6-dioxopiperidin-3-yl)-1-methyl-1H-indazol-7-yl)oxy)acetamide B153;
N-(2,5-difluorobenzyl)-2-((3-(2,6-dioxopiperidin-3-yl)-1-methyl-1H-indazol-7-yl)oxy)acetamide B154;
2-((3-(2,6-dioxopiperidin-3-yl)-1-methyl-1H-indazol-7-yl)oxy)-N-((1-methyl-pyrrolidin-3-yl)methyl)acetamide B155;
N-((4-benzylmorpholin-2-yl)methyl)-2-((3-(2,6-dioxopiperidin-3-yl)-1-methyl-1H-indazol-7-yl)oxy)acetamide B156;
N-((1,5-dimethyl-1H-pyrazol-3-yl)methyl)-2-((3-(2,6-dioxopiperidin-3-yl)-1-methyl-1H-indazol-7-yl)oxy)acetamide B157;
2-((3-(2,6-dioxopiperidin-3-yl)-1-methyl-1H-indazol-7-yl)oxy)-N-neopentyl-acetamide B158;
2-((3-(2,6-dioxopiperidin-3-yl)-1-methyl-1H-indazol-7-yl)oxy)-N-(3-methyl-1H-pyrazol-5-yl)acetamide B159;
2-((3-(2,6-dioxopiperidin-3-yl)-1-methyl-1H-indazol-7-yl)oxy)-N-(4-(morpholinomethyl)benzyl)acetamide B160;
2-((3-(2,6-dioxopiperidin-3-yl)-1-methyl-1H-indazol-7-yl)oxy)-N-(tetrahydro-2H-pyran-4-yl)acetamide B161;
N-(3,5-dichlorobenzyl)-2-((3-(2,6-dioxopiperidin-3-yl)-1-methyl-1H-indazol-7-yl)oxy)acetamide B162;
N-(3,5-difluorobenzyl)-2-((3-(2,6-dioxopiperidin-3-yl)-1-methyl-1H-indazol-7-yl)oxy)acetamide B163;
2-((3-(2,6-dioxopiperidin-3-yl)-1-methyl-1H-indazol-7-yl)oxy)-N-(oxetan-2-yl-methyl)acetamide B164;
N-(2-(cyclopentyloxy)ethyl)-2-((3-(2,6-dioxopiperidin-3-yl)-1-methyl-1H-indazol-7-yl)oxy)acetamide B165;
N-(cyclopentylmethyl)-2-((3-(2,6-dioxopiperidin-3-yl)-1-methyl-1H-indazol-7-yl)oxy)acetamide B166;
N-cyclobutyl-2-((3-(2,6-dioxopiperidin-3-yl)-1-methyl-1H-indazol-7-yl)oxy)-acetamide B167;
2-((3-(2,6-dioxopiperidin-3-yl)-1-methyl-1H-indazol-7-yl)oxy)-N-(thiazol-2-yl-methyl)acetamide B168;
N-(4-(1H-pyrazol-1-yl)pyridin-2-yl)-2-((3-(2,6-dioxopiperidin-3-yl)-1-methyl-1H-indazol-7-yl)oxy)acetamide B169;
2-((3-(2,6-dioxopiperidin-3-yl)-1-methyl-1H-indazol-7-yl)oxy)-N-(3-isopropoxy-phenyl)acetamide B170;
2-((3-(2,6-dioxopiperidin-3-yl)-1-methyl-1H-indazol-7-yl)oxy)-N-(3-(oxazol-5-yl)phenyl)acetamide B171;
2-((3-(2,6-dioxopiperidin-3-yl)-1-methyl-1H-indazol-7-yl)oxy)-N-(4-hydroxy-benzyl)acetamide B172;
2-((3-(2,6-dioxopiperidin-3-yl)-1-methyl-1H-indazol-7-yl)oxy)-N-(thiophen-2-yl-methyl)acetamide B173;
N-cyclopropyl-2-((3-(2,6-dioxopiperidin-3-yl)-1-methyl-1H-indazol-7-yl)oxy)-acetamide B174;
N-(3-(tert-butyl)phenyl)-2-((3-(2,6-dioxopiperidin-3-yl)-1-methyl-1H-indazol-7-yl)oxy)acetamide B175;
N-(cyclopropylmethyl)-2-((3-(2,6-dioxopiperidin-3-yl)-1-methyl-1H-indazol-7-yl)oxy)acetamide B176;
N-(3-cyclopropyl-1H-pyrazol-5-yl)-2-((3-(2,6-dioxopiperidin-3-yl)-1-methyl-1H-indazol-7-yl)oxy)acetamide B177;
N-(3-(1H-imidazol-1-yl)phenyl)-2-((3-(2,6-dioxopiperidin-3-yl)-1-methyl-1H-indazol-7-yl)oxy)acetamide B178;
N-cyclohexyl-2-((3-(2,6-dioxopiperidin-3-yl)-1-methyl-1H-indazol-7-yl)oxy)-acetamide B179;
4-amino-5-(4-(2-((3-(2,6-dioxopiperidin-3-yl)-1-methyl-1H-indazol-7-yl)oxy)-acetyl)piperazine-1-carbonyl) isothiazole-3-carboxamide B201;
5-(4-(2-((3-(2,6-dioxopiperidin-3-yl)-1-methyl-1H-indazol-7-yl)oxy)acetyl)-piperazine-1-carbonyl)-1H-pyrrole-2-carboxylic acid B202;
3-(7-(2-(4-((1,2-dimethyl-1H-imidazol-5-yl) sulfonyl)piperazin-1-yl)-2-oxo-ethoxy)-1-methyl-1H-indazol-3-yl)piperidine-2,6-dione B203;
3-(4-(2-((3-(2,6-dioxopiperidin-3-yl)-1-methyl-1H-indazol-7-yl)oxy)acetyl)-piperazine-1-carbonyl)-1-methyl-1H-pyrazole-5-carboxylic acid B204;
3-(7-(2-(4-((5-(5-chloro-1,2,4-thiadiazol-3-yl)thiophen-2-yl) sulfonyl)piperazin-1-yl)-2-oxoethoxy)-1-methyl-1H-indazol-3-yl)piperidine-2,6-dione B205;
4-amino-5-(4-(2-((3-(2,6-dioxopiperidin-3-yl)-1-methyl-1H-indazol-7-yl)oxy)-acetyl)piperazine-1-carbonyl)-N-isopropylisothiazole-3-carboxamide B206;
3-(1-methyl-7-(2-oxo-2-(4-((tetrahydro-2H-pyran-4-yl) sulfonyl)piperazin-1-yl)-ethoxy)-1H-indazol-3-yl)piperidine-2,6-dione B207;
3-(1-methyl-7-(2-(4-((1-methyl-1H-imidazol-4-yl) sulfonyl)piperazin-1-yl)-2-oxo-ethoxy)-1H-indazol-3-yl)piperidine-2,6-dione B208;
4-amino-5-(4-(2-((3-(2,6-dioxopiperidin-3-yl)-1-methyl-1H-indazol-7-yl)oxy)-acetyl)piperazine-1-carbonyl)-N-ethylisothiazole-3-carboxamide B209;
3-(1-methyl-7-(2-oxo-2-(4-((1,3,5-trimethyl-1H-pyrazol-4-yl) sulfonyl)piperazin-1-yl)ethoxy)-1H-indazol-3-yl)piperidine-2,6-dione B210;
3-(1-methyl-7-(2-oxo-2-(4-((2,2,4,6,7-pentamethyl-2,3-dihydrobenzofuran-5-yl)-sulfonyl)piperazin-1-yl)ethoxy)-1H-indazol-3-yl)piperidine-2,6-dione B211;
3-(7-(2-(4-(2-(cyclopropylamino) thiazole-5-carbonyl)piperazin-1-yl)-2-oxo-ethoxy)-1-methyl-1H-indazol-3-yl)piperidine-2,6-dione B212;
3-(1-methyl-7-(2-(4-(3-methylisothiazole-5-carbonyl)piperazin-1-yl)-2-oxo-ethoxy)-1H-indazol-3-yl)piperidine-2,6-dione B213;
3-(7-(2-(4-((1-ethyl-3-methyl-1H-pyrazol-5-yl) sulfonyl)piperazin-1-yl)-2-oxo-ethoxy)-1-methyl-1H-indazol-3-yl)piperidine-2,6-dione B214;
3-(7-(2-(4-((3,5-dimethylisoxazol-4-yl) sulfonyl)piperazin-1-yl)-2-oxoethoxy)-1-methyl-1H-indazol-3-yl)piperidine-2,6-dione B215;
3-(7-(2-(4-((1-ethyl-1H-pyrazol-4-yl) sulfonyl)piperazin-1-yl)-2-oxoethoxy)-1-methyl-1H-indazol-3-yl)piperidine-2,6-dione B216;
3-(7-(2-(4-((cyclobutylmethyl) sulfonyl)piperazin-1-yl)-2-oxoethoxy)-1-methyl-1H-indazol-3-yl)piperidine-2,6-dione B217;
3-(1-methyl-7-(2-(4-((3-(5-methyl-1,2,4-oxadiazol-3-yl)phenyl) sulfonyl)-piperazin-1-yl)-2-oxoethoxy)-1H-indazol-3-yl)piperidine-2,6-dione B218;
3-(7-(2-(4-(1,2,4-oxadiazole-3-carbonyl)piperazin-1-yl)-2-oxoethoxy)-1-methyl-1H-indazol-3-yl)piperidine-2,6-dione B219;
3-(7-(2-(4-(6,7-dihydro-4H-pyrazolo[5,1-c][1,4]oxazine-2-carbonyl)piperazin-1-yl)-2-oxoethoxy)-1-methyl-1H-indazol-3-yl)piperidine-2,6-dione B220;
3-(7-(2-(4-(2,4-dimethyloxazole-5-carbonyl)piperazin-1-yl)-2-oxoethoxy)-1-methyl-1H-indazol-3-yl)piperidine-2,6-dione B221;
3-(1-methyl-7-(2-oxo-2-(4-((4-(trifluoromethoxy)phenyl) sulfonyl)piperazin-1-yl)ethoxy)-1H-indazol-3-yl)piperidine-2,6-dione B222;
3-(1-methyl-7-(2-(4-(4-methylthiazole-2-carbonyl)piperazin-1-yl)-2-oxoethoxy)-1H-indazol-3-yl)piperidine-2,6-dione B223;
5-(4-(2-((3-(2,6-dioxopiperidin-3-yl)-1-methyl-1H-indazol-7-yl)oxy)acetyl)-piperazine-1-carbonyl)-1H-pyrrole-3-carbonitrile B224;
3-(1-methyl-7-(2-(4-(2-methyl-3H-pyrazolo[1,5-b][1,2,4]triazole-7-carbonyl)-piperazin-1-yl)-2-oxoethoxy)-1H-indazol-3-yl)piperidine-2,6-dione B225;
methyl 5-(4-(2-((3-(2,6-dioxopiperidin-3-yl)-1-methyl-1H-indazol-7-yl)oxy)-acetyl)piperazine-1-carbonyl)-1-methyl-1H-pyrazole-3-carboxylate B226;
ethyl 5-(4-(2-((3-(2,6-dioxopiperidin-3-yl)-1-methyl-1H-indazol-7-yl)oxy)-acetyl)piperazine-1-carbonyl)-1H-pyrrole-2-carboxylate B227;
3-(7-(2-(4-(2,5-dimethyloxazole-4-carbonyl)piperazin-1-yl)-2-oxoethoxy)-1-methyl-1H-indazol-3-yl)piperidine-2,6-dione B228;
3-(1-methyl-7-(2-oxo-2-(4-(2,4,5,6-tetrahydrocyclopenta[c]pyrrole-1-carbonyl)-piperazin-1-yl)ethoxy)-1H-indazol-3-yl)piperidine-2,6-dione B229;
3-(1-methyl-7-(2-oxo-2-(4-((3-oxo-3,4-dihydro-2H-benzo[b][1,4]oxazin-6-yl)-sulfonyl)piperazin-1-yl)ethoxy)-1H-indazol-3-yl)piperidine-2,6-dione B230;
3-(1-methyl-7-(2-oxo-2-(4-((2-oxoindolin-6-yl) sulfonyl)piperazin-1-yl)ethoxy)-1H-indazol-3-yl)piperidine-2,6-dione B231;
3-(7-(2-(4-((2-chloro-5-methylpyridin-3-yl) sulfonyl)piperazin-1-yl)-2-oxo-ethoxy)-1-methyl-1H-indazol-3-yl)piperidine-2,6-dione B232;
3-(7-(2-(4-(4-amino-1,2,5-oxadiazole-3-carbonyl)piperazin-1-yl)-2-oxoethoxy)-1-methyl-1H-indazol-3-yl)piperidine-2,6-dione B233;
4-((4-(2-((3-(2,6-dioxopiperidin-3-yl)-1-methyl-1H-indazol-7-yl)oxy)acetyl)-piperazin-1-yl) sulfonyl)benzamide B234;
3-(7-(2-(4-((4-(tert-butyl)phenyl) sulfonyl)piperazin-1-yl)-2-oxoethoxy)-1-methyl-1H-indazol-3-yl)piperidine-2,6-dione B235;
ethyl 4-(4-(2-((3-(2,6-dioxopiperidin-3-yl)-1-methyl-1H-indazol-7-yl)oxy)acetyl)-piperazine-1-carbonyl)-1H-pyrrole-2-carboxylate B236;
3-(7-(2-(4-((1,5-dimethyl-1H-pyrazol-3-yl) sulfonyl)piperazin-1-yl)-2-oxo-ethoxy)-1-methyl-1H-indazol-3-yl)piperidine-2,6-dione B237;
3-(1-methyl-7-(2-(4-((1-methyl-1H-pyrazol-4-yl) sulfonyl)piperazin-1-yl)-2-oxo-ethoxy)-1H-indazol-3-yl)piperidine-2,6-dione B238;
3-(1-methyl-7-(2-(4-(5-methylthiazole-2-carbonyl)piperazin-1-yl)-2-oxoethoxy)-1H-indazol-3-yl)piperidine-2,6-dione B239;
3-(7-(2-(4-((4-fluorophenyl) sulfonyl)piperazin-1-yl)-2-oxoethoxy)-1-methyl-1H-indazol-3-yl)piperidine-2,6-dione B240;
3-(1-methyl-7-(2-oxo-2-(4-(thiophen-2-ylsulfonyl)piperazin-1-yl)ethoxy)-1H-indazol-3-yl)piperidine-2,6-dione B241;
3-(7-(2-(4-(4-amino-1-methyl-1H-pyrazole-5-carbonyl)piperazin-1-yl)-2-oxo-ethoxy)-1-methyl-1H-indazol-3-yl)piperidine-2,6-dione B242;
5-(4-(2-((3-(2,6-dioxopiperidin-3-yl)-1-methyl-1H-indazol-7-yl)oxy)acetyl)-piperazine-1-carbonyl) thiazole-2-carbonitrile B243;
3-(1-methyl-7-((1-(oxetane-3-carbonyl)piperidin-4-yl)oxy)-1H-indazol-3-yl)-piperidine-2,6-dione C001;
3-(7-((1-(4-amino-1-methyl-1H-pyrazole-3-carbonyl)piperidin-4-yl)oxy)-1-methyl-1H-indazol-3-yl)piperidine-2,6-dione C002;
3-(1-methyl-7-((1-(2-(4-oxo-2-thioxothiazolidin-3-yl)acetyl)piperidin-4-yl)oxy)-1H-indazol-3-yl)piperidine-2,6-dione C003;
3-(1-methyl-7-((1-(4-oxo-1,4-dihydropyrrolo[1,2-b]pyridazine-3-carbonyl)-piperidin-4-yl)oxy)-1H-indazol-3-yl)piperidine-2,6-dione C004;
3-(1-methyl-7-((1-(3-(4-(trifluoromethyl)phenyl) propioloyl)piperidin-4-yl)oxy)-1H-indazol-3-yl)piperidine-2,6-dione C005;
5-(4-((3-(2,6-dioxopiperidin-3-yl)-1-methyl-1H-indazol-7-yl)oxy) piperidine-1-carbonyl)-1H-pyrrole-2-carboxamide C006;
3-(7-((1-(4-amino-1-methyl-1H-pyrazole-5-carbonyl)piperidin-4-yl)oxy)-1-methyl-1H-indazol-3-yl)piperidine-2,6-dione C007;
3-(1-methyl-7-((1-(methylglycyl)piperidin-4-yl)oxy)-1H-indazol-3-yl)piperidine-2,6-dione C008;
2-(4-((3-(2,6-dioxopiperidin-3-yl)-1-methyl-1H-indazol-7-yl)oxy) piperidine-1-carbonyl)-1H-pyrrole-3-carbonitrile C009;
3-(7-((1-(azetidine-3-carbonyl)piperidin-4-yl)oxy)-1-methyl-1H-indazol-3-yl)-piperidine-2,6-dione C010;
(E)-3-(1-methyl-7-((1-(3-(4-(trifluoromethyl)phenyl) acryloyl)piperidin-4-yl)oxy)-1H-indazol-3-yl)piperidine-2,6-dione C011;
ethyl 4-(4-((3-(2,6-dioxopiperidin-3-yl)-1-methyl-1H-indazol-7-yl)oxy)-piperidine-1-carbonyl)-1H-pyrrole-2-carboxylate C012;
3-(1-methyl-7-((1-(2-phenylthiazole-4-carbonyl)piperidin-4-yl)oxy)-1H-indazol-3-yl)piperidine-2,6-dione C013;
3-(7-((1-(5-methoxypicolinoyl)piperidin-4-yl)oxy)-1-methyl-1H-indazol-3-yl)-piperidine-2,6-dione C014;
3-(7-((1-(2-((2,4-difluorophenyl)thio) acetyl)piperidin-4-yl)oxy)-1-methyl-1H-indazol-3-yl)piperidine-2,6-dione C015;
4-(4-((3-(2,6-dioxopiperidin-3-yl)-1-methyl-1H-indazol-7-yl)oxy) piperidine-1-carbonyl)-3-fluorobenzonitrile C016;
3-(1-methyl-7-((1-(1-methyl-3-(trifluoromethyl)-1H-pyrazole-4-carbonyl)-piperidin-4-yl)oxy)-1H-indazol-3-yl)piperidine-2,6-dione C017;
3-(1-methyl-7-((1-(2-((phenylthio)methyl)benzoyl)piperidin-4-yl)oxy)-1H-indazol-3-yl)piperidine-2,6-dione C018;
3-(1-methyl-7-((1-(1-methyl-5-(trifluoromethyl)-1H-pyrrole-2-carbonyl)-piperidin-4-yl)oxy)-1H-indazol-3-yl)piperidine-2,6-dione C019;
3-(7-((1-(3-methoxythiophene-2-carbonyl)piperidin-4-yl)oxy)-1-methyl-1H-indazol-3-yl)piperidine-2,6-dione C020;
3-(1-methyl-7-((1-(pyrrolo[2,1-b]thiazole-6-carbonyl)piperidin-4-yl)oxy)-1H-indazol-3-yl)piperidine-2,6-dione C021;
3-(7-((1-(4,5-dimethylthiophene-3-carbonyl)piperidin-4-yl)oxy)-1-methyl-1H-indazol-3-yl)piperidine-2,6-dione C022;
3-(1-methyl-7-((1-(1-methyl-1H-pyrrole-2-carbonyl)piperidin-4-yl)oxy)-1H-indazol-3-yl)piperidine-2,6-dione C023;
3-(7-((1-(2,2-difluoro-2-(4-fluorophenyl)acetyl)piperidin-4-yl)oxy)-1-methyl-1H-indazol-3-yl)piperidine-2,6-dione C024;
3-(7-((1-(2-cyclobutylacetyl)piperidin-4-yl)oxy)-1-methyl-1H-indazol-3-yl)-piperidine-2,6-dione C025;
3-(7-((1-(bicyclo[1.1.1]pentane-1-carbonyl)piperidin-4-yl)oxy)-1-methyl-1H-indazol-3-yl)piperidine-2,6-dione C026;
3-(7-((1-(4-isopropoxybenzoyl)piperidin-4-yl)oxy)-1-methyl-1H-indazol-3-yl)-piperidine-2,6-dione C027;
3-(7-((1-(1,1-dioxidothietane-3-carbonyl)piperidin-4-yl)oxy)-1-methyl-1H-indazol-3-yl)piperidine-2,6-dione C028;
3-(7-((1-(imidazo[1,2-a]pyridine-6-carbonyl)piperidin-4-yl)oxy)-1-methyl-1H-indazol-3-yl)piperidine-2,6-dione C029;
3-(1-methyl-7-((1-(4-phenoxybenzoyl)piperidin-4-yl)oxy)-1H-indazol-3-yl)-piperidine-2,6-dione C030;
3-(1-methyl-7-((1-(4-oxo-4H-chromene-2-carbonyl)piperidin-4-yl)oxy)-1H-indazol-3-yl)piperidine-2,6-dione C031;
3-(1-methyl-7-((1-(3-phenylpropioloyl)piperidin-4-yl)oxy)-1H-indazol-3-yl)-piperidine-2,6-dione C032;
3-(7-((1-(2,4-dimethylthiazole-5-carbonyl)piperidin-4-yl)oxy)-1-methyl-1H-indazol-3-yl)piperidine-2,6-dione C033;
3-(1-methyl-7-((1-(2-(p-tolylthio) acetyl)piperidin-4-yl)oxy)-1H-indazol-3-yl)-piperidine-2,6-dione C034;
2-(4-((3-(2,6-dioxopiperidin-3-yl)-1-methyl-1H-indazol-7-yl)oxy) piperidin-1-yl)-N-(2-methoxyphenyl)-2-oxoacetamide C₀₃₅;
3-(7-((1-(1,5-dimethyl-1H-pyrazole-4-carbonyl)piperidin-4-yl)oxy)-1-methyl-1H-indazol-3-yl)piperidine-2,6-dione C036;
3-(7-((1-(1-hydroxycyclopropane-1-carbonyl)piperidin-4-yl)oxy)-1-methyl-1H-indazol-3-yl)piperidine-2,6-dione C037;
3-(7-((1-(3-(difluoromethyl)-5-fluoro-1-methyl-1H-pyrazole-4-carbonyl)-piperidin-4-yl)oxy)-1-methyl-1H-indazol-3-yl)piperidine-2,6-dione C038;
N-(2-(4-((3-(2,6-dioxopiperidin-3-yl)-1-methyl-1H-indazol-7-yl)oxy) piperidin-1-yl)-2-oxoethyl)-2-phenylacetamide C₀₃₉;
3-(1-methyl-7-((1-(4,5,6,7-tetrahydro-1H-indazole-3-carbonyl)piperidin-4-yl)-oxy)-1H-indazol-3-yl)piperidine-2,6-dione C040;
3-(1-methyl-7-((1-(6-methylbenzo[b]thiophene-2-carbonyl)piperidin-4-yl)oxy)-1H-indazol-3-yl)piperidine-2,6-dione C041;
3-(7-((1-(1-(hydroxymethyl)cyclopropane-1-carbonyl)piperidin-4-yl)oxy)-1-methyl-1H-indazol-3-yl)piperidine-2,6-dione C042;
3-(1-methyl-7-((1-(3-phenoxybenzoyl)piperidin-4-yl)oxy)-1H-indazol-3-yl)-piperidine-2,6-dione C043;
3-(7-((1-(3-hydroxy-4-methoxybenzoyl)piperidin-4-yl)oxy)-1-methyl-1H-indazol-3-yl)piperidine-2,6-dione C044;
3-(7-((1-(4′-fluoro-[1,1,′-biphenyl]-3-carbonyl)piperidin-4-yl)oxy)-1-methyl-1H-indazol-3-yl)piperidine-2,6-dione C045;
3-(7-((1-(5-amino-1-phenyl-1H-pyrazole-4-carbonyl)piperidin-4-yl)oxy)-1-methyl-1H-indazol-3-yl)piperidine-2,6-dione C046;
3-(7-((1-(6,7-dihydro-4H-pyrazolo[5,1-c][1,4]oxazine-2-carbonyl)piperidin-4-yl)-oxy)-1-methyl-1H-indazol-3-yl)piperidine-2,6-dione C047;
4-amino-5-(4-((3-(2,6-dioxopiperidin-3-yl)-1-methyl-1H-indazol-7-yl)oxy)-piperidine-1-carbonyl) isothiazole-3-carboxamide C₀₄₈;
3-(1-methyl-7-((1-(5-(pyridin-3-yl)-1H-imidazole-4-carbonyl)piperidin-4-yl)oxy)-1H-indazol-3-yl)piperidine-2,6-dione C049;
3-(1-methyl-7-((1-(1-methyl-4-oxo-1,4-dihydropyrrolo[1,2-b]pyridazine-3-carbonyl)piperidin-4-yl)oxy)-1H-indazol-3-yl)piperidine-2,6-dione C050;
3-(7-((1-(6-ethoxypicolinoyl)piperidin-4-yl)oxy)-1-methyl-1H-indazol-3-yl)-piperidine-2,6-dione C051;
3-(1-methyl-7-((1-(2,4,5,6-tetrahydrocyclopenta[c]pyrrole-1-carbonyl)piperidin-4-yl)oxy)-1H-indazol-3-yl)piperidine-2,6-dione C052;
3-amino-5-(4-((3-(2,6-dioxopiperidin-3-yl)-1-methyl-1H-indazol-7-yl)oxy)-piperidine-1-carbonyl)benzonitrile C053;
3-(7-((1-(2-(benzyloxy)-2-methylpropanoyl)piperidin-4-yl)oxy)-1-methyl-1H-indazol-3-yl)piperidine-2,6-dione C054;
3-(7-((1-(1H-pyrazole-4-carbonyl)piperidin-4-yl)oxy)-1-methyl-1H-indazol-3-yl)-piperidine-2,6-dione C055;
3-(7-((1-(²H-indazole-5-carbonyl)piperidin-4-yl)oxy)-1-methyl-1H-indazol-3-yl)-piperidine-2,6-dione C056;
3-(7-((1-(1-(tert-butyl)-1H-1,2,3-triazole-4-carbonyl)piperidin-4-yl)oxy)-1-methyl-1H-indazol-3-yl)piperidine-2,6-dione C057;
3-(1-methyl-7-((1-(5-(pyridin-3-yl)-1H-pyrazole-3-carbonyl)piperidin-4-yl)oxy)-1H-indazol-3-yl)piperidine-2,6-dione C058;
3-(7-((1-(2-mercapto-1-methyl-1H-imidazole-5-carbonyl)piperidin-4-yl)oxy)-1-methyl-1H-indazol-3-yl)piperidine-2,6-dione C059;
3-(7-((1-(3-(benzo[d][1,3]dioxol-5-yl) acryloyl)piperidin-4-yl)oxy)-1-methyl-1H-indazol-3-yl)piperidine-2,6-dione C060;
3-(7-((1-(4-amino-1,2,5-oxadiazole-3-carbonyl)piperidin-4-yl)oxy)-1-methyl-1H-indazol-3-yl)piperidine-2,6-dione C061;
3-(7-((1-(2-aminothiazole-4-carbonyl)piperidin-4-yl)oxy)-1-methyl-1H-indazol-3-yl)piperidine-2,6-dione C062;
3-(7-((1-(4-benzyl-5-oxomorpholine-3-carbonyl)piperidin-4-yl)oxy)-1-methyl-1H-indazol-3-yl)piperidine-2,6-dione C063;
3-(7-((1-(imidazo[2,1-b]thiazole-5-carbonyl)piperidin-4-yl)oxy)-1-methyl-1H-indazol-3-yl)piperidine-2,6-dione C064;
3-(1-methyl-7-((1-(3-(phenylsulfonyl) propanoyl)piperidin-4-yl)oxy)-1H-indazol-3-yl)piperidine-2,6-dione C065;
5-(4-((3-(2,6-dioxopiperidin-3-yl)-1-methyl-1H-indazol-7-yl)oxy) piperidine-1-carbonyl) pyrazine-2-carbonitrile C066;
3-(7-((1-(4-hydroxycyclohexane-1-carbonyl)piperidin-4-yl)oxy)-1-methyl-1H-indazol-3-yl)piperidine-2,6-dione C067;
3-(1-methyl-7-((1-(4,5,6,7-tetrahydrobenzo[b]thiophene-2-carbonyl)piperidin-4-yl)oxy)-1H-indazol-3-yl)piperidine-2,6-dione C068;
3-(7-((1-(4,6-dihydroxypyridazine-3-carbonyl)piperidin-4-yl)oxy)-1-methyl-1H-indazol-3-yl)piperidine-2,6-dione C069;
3-(7-((1-(2,3-dihydrobenzofuran-7-carbonyl)piperidin-4-yl)oxy)-1-methyl-1H-indazol-3-yl)piperidine-2,6-dione C070;
3-(7-((1-(6-methoxy-1H-indazole-3-carbonyl)piperidin-4-yl)oxy)-1-methyl-1H-indazol-3-yl)piperidine-2,6-dione C071;
(E)-3-(7-((1-(3-(3,4-difluorophenyl) acryloyl)piperidin-4-yl)oxy)-1-methyl-1H-indazol-3-yl)piperidine-2,6-dione C072;
3-(1-methyl-7-((1-(3-phenylcyclobutane-1-carbonyl)piperidin-4-yl)oxy)-1H-indazol-3-yl)piperidine-2,6-dione C073;
3-(1-methyl-7-((1-(2-methyl-1H-imidazole-5-carbonyl)piperidin-4-yl)oxy)-1H-indazol-3-yl)piperidine-2,6-dione C074;
3-(7-((1-(2-(cyclopropylamino) thiazole-5-carbonyl)piperidin-4-yl)oxy)-1-methyl-1H-indazol-3-yl)piperidine-2,6-dione C075;
5-(4-((3-(2,6-dioxopiperidin-3-yl)-1-methyl-1H-indazol-7-yl)oxy) piperidine-1-carbonyl) furan-3-carbonitrile C076;
3-(7-((1-(2-cyclopropylacetyl)piperidin-4-yl)oxy)-1-methyl-1H-indazol-3-yl)-piperidine-2,6-dione C077;
4-(4-((3-(2,6-dioxopiperidin-3-yl)-1-methyl-1H-indazol-7-yl)oxy) piperidine-1-carbonyl)piperidine-1-carboximidamide C₀₇₈;
N-cyclohexyl-2-(4-((3-(2,6-dioxopiperidin-3-yl)-1-methyl-1H-indazol-7-yl)oxy)-piperidin-1-yl)-2-oxoacetamide C₀₇₉;
5-(4-((3-(2,6-dioxopiperidin-3-yl)-1-methyl-1H-indazol-7-yl)oxy) piperidine-1-carbonyl)-1H-pyrrole-3-carbonitrile C080;
3-(7-((1-(4-(1H-imidazol-2-yl)benzoyl)piperidin-4-yl)oxy)-1-methyl-1H-indazol-3-yl)piperidine-2,6-dione C081;
3-(7-((1-(5-amino-2-methylthiazole-4-carbonyl)piperidin-4-yl)oxy)-1-methyl-1H-indazol-3-yl)piperidine-2,6-dione C082;
N-(4-(4-((3-(2,6-dioxopiperidin-3-yl)-1-methyl-1H-indazol-7-yl)oxy) piperidine-1-carbonyl)phenyl)benzenesulfonamide C₀₈₃;
3-(7-((1-(cyclobut-1-ene-1-carbonyl)piperidin-4-yl)oxy)-1-methyl-1H-indazol-3-yl)piperidine-2,6-dione C084;
3-(1-methyl-7-((1-(4-oxo-4,5,6,7-tetrahydropyrazolo[1,5-a]pyrazine-2-carbonyl)-piperidin-4-yl)oxy)-1H-indazol-3-yl)piperidine-2,6-dione C085;
3-(7-((1-(3-(3-fluorophenyl) propioloyl)piperidin-4-yl)oxy)-1-methyl-1H-indazol-3-yl)piperidine-2,6-dione C086;
3-(7-((1-(4H-thieno[3,2-b]pyrrole-5-carbonyl)piperidin-4-yl)oxy)-1-methyl-1H-indazol-3-yl)piperidine-2,6-dione C087;
3-(1-methyl-7-((1-(2-methyl-4-(trifluoromethyl) furan-3-carbonyl)piperidin-4-yl)-oxy)-1H-indazol-3-yl)piperidine-2,6-dione C088;
3-(7-((1-(2,5-dimethyloxazole-4-carbonyl)piperidin-4-yl)oxy)-1-methyl-1H-indazol-3-yl)piperidine-2,6-dione C089;
3-(7-((1-(2-(2-mercapto-4-methylthiazol-5-yl)acetyl)piperidin-4-yl)oxy)-1-methyl-1H-indazol-3-yl)piperidine-2,6-dione C090;
3-(7-((1-(4-hydroxy-3-(trifluoromethyl)benzoyl)piperidin-4-yl)oxy)-1-methyl-1H-indazol-3-yl)piperidine-2,6-dione C091;
3-(7-((1-(1H-pyrrolo[2,3-b]pyridine-3-carbonyl)piperidin-4-yl)oxy)-1-methyl-1H-indazol-3-yl)piperidine-2,6-dione C092;
4-(4-((3-(2,6-dioxopiperidin-3-yl)-1-methyl-1H-indazol-7-yl)oxy) piperidine-1-carbonyl)tetrahydro-2H-pyran-4-carbonitrile C093;
3-(7-((1-(3-hydroxypropanoyl)piperidin-4-yl)oxy)-1-methyl-1H-indazol-3-yl)-piperidine-2,6-dione C094;
3-(1-methyl-7-((1-(pent-2-ynoyl)piperidin-4-yl)oxy)-1H-indazol-3-yl)piperidine-2,6-dione C095;
3-(7-((1-(2-isopropylisonicotinoyl)piperidin-4-yl)oxy)-1-methyl-1H-indazol-3-yl)piperidine-2,6-dione C096;
2-(2-(4-((3-(2,6-dioxopiperidin-3-yl)-1-methyl-1H-indazol-7-yl)oxy) piperidin-1-yl)-2-oxoethoxy)benzonitrile C097;
3-(7-((1-(6,7-dihydro-5H-pyrazolo[5,1-b][1,3]oxazine-2-carbonyl)piperidin-4-yl)oxy)-1-methyl-1H-indazol-3-yl)piperidine-2,6-dione C098;
3-(1-methyl-7-((1-(4-oxo-4H-chromene-3-carbonyl)piperidin-4-yl)oxy)-1H-indazol-3-yl)piperidine-2,6-dione C099;
3-(1-methyl-7-((1-(3-methylisothiazole-5-carbonyl)piperidin-4-yl)oxy)-1H-indazol-3-yl)piperidine-2,6-dione C100;
3-(7-((1-(2-((4-chlorophenyl)thio) acetyl)piperidin-4-yl)oxy)-1-methyl-1H-indazol-3-yl)piperidine-2,6-dione C101;
3-(7-((1-(imidazo[1,2-a]pyridine-3-carbonyl)piperidin-4-yl)oxy)-1-methyl-1H-indazol-3-yl)piperidine-2,6-dione C102;
3-(1-methyl-7-((1-(5-methylthiophene-2-carbonyl)piperidin-4-yl)oxy)-1H-indazol-3-yl)piperidine-2,6-dione C103;
3-(7-((1-(5-isopropyl-1H-pyrazole-3-carbonyl)piperidin-4-yl)oxy)-1-methyl-1H-indazol-3-yl)piperidine-2,6-dione C104;
3-(7-((1-(4-(tert-butyl)benzoyl)piperidin-4-yl)oxy)-1-methyl-1H-indazol-3-yl)-piperidine-2,6-dione C105;
3-(1-methyl-7-((1-(4,5,6,7-tetrahydrobenzo[b]thiophene-3-carbonyl)piperidin-4-yl)oxy)-1H-indazol-3-yl)piperidine-2,6-dione C106;
3-(1-methyl-7-((1-(thiazole-5-carbonyl)piperidin-4-yl)oxy)-1H-indazol-3-yl)-piperidine-2,6-dione C107;
3-(1-methyl-7-((1-(oxazole-4-carbonyl)piperidin-4-yl)oxy)-1H-indazol-3-yl)-piperidine-2,6-dione C108;
(E)-3-(7-((1-(3-(4-aminophenyl) acryloyl)piperidin-4-yl)oxy)-1-methyl-1H-indazol-3-yl)piperidine-2,6-dione C109;
3-(7-((1-(1-(6-(cyclopropylamino)pyrimidin-4-yl)piperidine-4-carbonyl)-piperidin-4-yl)oxy)-1-methyl-1H-indazol-3-yl)piperidine-2,6-dione C110;
3-(1-methyl-7-((1-(oxazole-2-carbonyl)piperidin-4-yl)oxy)-1H-indazol-3-yl)-piperidine-2,6-dione C111;
2-(4-((3-(2,6-dioxopiperidin-3-yl)-1-methyl-1H-indazol-7-yl)oxy) piperidin-1-yl)-N-(4-methoxyphenyl)-2-oxoacetamide C₁₁₂;
3-(7-((1-(1,2,4-oxadiazole-3-carbonyl)piperidin-4-yl)oxy)-1-methyl-1H-indazol-3-yl)piperidine-2,6-dione C113;
3-(1-methyl-7-((1-(2-(piperazin-1-yl)acetyl)piperidin-4-yl)oxy)-1H-indazol-3-yl)-piperidine-2,6-dione C114;
(E)-3-(1-methyl-7-((1-(3-(thiazol-2-yl) acryloyl)piperidin-4-yl)oxy)-1H-indazol-3-yl)piperidine-2,6-dione C115;
(E)-3-(1-methyl-7-((1-(3-(5-methylthiophen-2-yl) acryloyl)piperidin-4-yl)oxy)-1H-indazol-3-yl)piperidine-2,6-dione C116;
1-(4-((3-(2,6-dioxopiperidin-3-yl)-1-methyl-1H-indazol-7-yl)oxy) piperidine-1-carbonyl)cyclobutane-1-carbonitrile C117;
3-(4-((3-(2,6-dioxopiperidin-3-yl)-1-methyl-1H-indazol-7-yl)oxy) piperidine-1-carbonyl)-5-methylbenzonitrile C118;
2-(4-((3-(2,6-dioxopiperidin-3-yl)-1-methyl-1H-indazol-7-yl)oxy) piperidin-1-yl)-N-(3-methoxyphenyl)-2-oxoacetamide C₁₁₉;
3-(7-((1-(5-chlorofuran-2-carbonyl)piperidin-4-yl)oxy)-1-methyl-1H-indazol-3-yl)piperidine-2,6-dione C120;
3-(1-methyl-7-((1-(2-(3-methylisoxazol-5-yl)acetyl)piperidin-4-yl)oxy)-1H-indazol-3-yl)piperidine-2,6-dione C121;
(E)-3-(1-methyl-7-((1-(pent-2-enoyl)piperidin-4-yl)oxy)-1H-indazol-3-yl)-piperidine-2,6-dione C122;
(E)-3-(7-((1-(3-(benzofuran-2-yl) acryloyl)piperidin-4-yl)oxy)-1-methyl-1H-indazol-3-yl)piperidine-2,6-dione C123;
3-(1-methyl-7-((1-(1-methylpiperidine-2-carbonyl)piperidin-4-yl)oxy)-1H-indazol-3-yl)piperidine-2,6-dione C124;
3-(7-((1-(4-(1H-pyrazol-1-yl)benzoyl)piperidin-4-yl)oxy)-1-methyl-1H-indazol-3-yl)piperidine-2,6-dione C5;
3-(7-((1-(2-(1,1-dioxidothiomorpholino) acetyl)piperidin-4-yl)oxy)-1-methyl-1H-indazol-3-yl)piperidine-2,6-dione C126;
1-(3-(4-((3-(2,6-dioxopiperidin-3-yl)-1-methyl-1H-indazol-7-yl)oxy) piperidin-1-yl)-3-oxopropyl) guanidine Cl₂₇;
1-(2-(4-((3-(2,6-dioxopiperidin-3-yl)-1-methyl-1H-indazol-7-yl)oxy) piperidin-1-yl)-2-oxoethyl) guanidine Cl₂₈;
3-(1-methyl-7-((1-(4-methylthiazole-2-carbonyl)piperidin-4-yl)oxy)-1H-indazol-3-yl)piperidine-2,6-dione C129;
3-(4-((3-(2,6-dioxopiperidin-3-yl)-1-methyl-1H-indazol-7-yl)oxy) piperidine-1-carbonyl) bicyclo[1.1.1]pentane-1-carbonitrile C130;
3-(7-((1-(2-amino-4-methylthiazole-5-carbonyl)piperidin-4-yl)oxy)-1-methyl-1H-indazol-3-yl)piperidine-2,6-dione C131;
3-(1-methyl-7-((1-(4-nitrobenzoyl)piperidin-4-yl)oxy)-1H-indazol-3-yl)-piperidine-2,6-dione C132;
3-(7-((1-(1-(2-fluorophenyl)-1H-pyrazole-4-carbonyl)piperidin-4-yl)oxy)-1-methyl-1H-indazol-3-yl)piperidine-2,6-dione C133;
3-(1-methyl-7-((1-(pyridazine-3-carbonyl)piperidin-4-yl)oxy)-1H-indazol-3-yl)-piperidine-2,6-dione C134;
4-amino-5-(4-((3-(2,6-dioxopiperidin-3-yl)-1-methyl-1H-indazol-7-yl)oxy)-piperidine-1-carbonyl)-N-ethylisothiazole-3-carboxamide C₁₃₅;
4-amino-5-(4-((3-(2,6-dioxopiperidin-3-yl)-1-methyl-1H-indazol-7-yl)oxy)-piperidine-1-carbonyl)-N-isopropylisothiazole-3-carboxamide C₁₃₆;
3-(1-methyl-7-((1-(2-(trifluoromethoxy)benzoyl)piperidin-4-yl)oxy)-1H-indazol-3-yl)piperidine-2,6-dione C137;
3-(7-((1-(2-(1H-pyrazol-1-yl)acetyl)piperidin-4-yl)oxy)-1-methyl-1H-indazol-3-yl)piperidine-2,6-dione C138;
3-(7-((1-(3-(tert-butyl) bicyclo[1.1.1]pentane-1-carbonyl)piperidin-4-yl)oxy)-1-methyl-1H-indazol-3-yl)piperidine-2,6-dione C139;
3-(1-methyl-7-((1-(tetrahydro-2H-thiopyran-4-carbonyl)piperidin-4-yl)oxy)-1H-indazol-3-yl)piperidine-2,6-dione C140;
3-(1-methyl-7-((1-(1-methyl-5-(trifluoromethyl)-1H-imidazole-4-carbonyl)-piperidin-4-yl)oxy)-1H-indazol-3-yl)piperidine-2,6-dione C141;
3-(7-((1-(2-(benzyloxy)acetyl)piperidin-4-yl)oxy)-1-methyl-1H-indazol-3-yl)-piperidine-2,6-dione C142;
3-(7-((1-(1-isopropyl-1H-pyrazole-5-carbonyl)piperidin-4-yl)oxy)-1-methyl-1H-indazol-3-yl)piperidine-2,6-dione C143;
3-(1-methyl-7-((1-(3-(trifluoromethoxy)benzoyl)piperidin-4-yl)oxy)-1H-indazol-3-yl)piperidine-2,6-dione C144;
3-(1-methyl-7-((1-(2-(thiophen-2-yl)acetyl)piperidin-4-yl)oxy)-1H-indazol-3-yl)piperidine-2,6-dione C145;
3-(1-methyl-7-((1-(2-methyl-2,4,5,6-tetrahydrocyclopenta[c]pyrazole-3-carbonyl)piperidin-4-yl)oxy)-1H-indazol-3-yl)piperidine-2,6-dione C146;
3-(1-methyl-7-((1-(thiophene-2-carbonyl)piperidin-4-yl)oxy)-1H-indazol-3-yl)-piperidine-2,6-dione C147;
4-(4-((3-(2,6-dioxopiperidin-3-yl)-1-methyl-1H-indazol-7-yl)oxy) piperidine-1-carbonyl)benzonitrile C148;
3-(7-((1-(cyclopentanecarbonyl)piperidin-4-yl)oxy)-1-methyl-1H-indazol-3-yl)-piperidine-2,6-dione C149;
3-(7-((1-(2-(dimethylamino)pyrimidine-4-carbonyl)piperidin-4-yl)oxy)-1-methyl-1H-indazol-3-yl)piperidine-2,6-dione C150;
3-(7-((1-(1-(tert-butyl)-1H-pyrazole-3-carbonyl)piperidin-4-yl)oxy)-1-methyl-1H-indazol-3-yl)piperidine-2,6-dione C151;
(E)-3-(7-((1-cinnamoylpiperidin-4-yl)oxy)-1-methyl-1H-indazol-3-yl)piperidine-2,6-dione C152;
5-(2-(4-((3-(2,6-dioxopiperidin-3-yl)-1-methyl-1H-indazol-7-yl)oxy) piperidin-1-yl)-2-oxoethyl)-1-methyl-1H-pyrrole-2-carbonitrile C153;
3-(1-methyl-7-((1-(thiazole-4-carbonyl)piperidin-4-yl)oxy)-1H-indazol-3-yl)-piperidine-2,6-dione C154;
3-(1-methyl-7-((1-(thiophene-3-carbonyl)piperidin-4-yl)oxy)-1H-indazol-3-yl)-piperidine-2,6-dione C155;
3-(7-((1-(1-acetylpiperidine-4-carbonyl)piperidin-4-yl)oxy)-1-methyl-1H-indazol-3-yl)piperidine-2,6-dione C156;
3-(7-((1-(3-isopropoxybenzoyl)piperidin-4-yl)oxy)-1-methyl-1H-indazol-3-yl)-piperidine-2,6-dione C157;
3-(1-methyl-7-((1-(pyrimidine-4-carbonyl)piperidin-4-yl)oxy)-1H-indazol-3-yl)-piperidine-2,6-dione C158;
3-(1-methyl-7-((1-(5-phenyloxazole-4-carbonyl)piperidin-4-yl)oxy)-1H-indazol-3-yl)piperidine-2,6-dione C159;
3-(7-((1-(2,4-dimethyloxazole-5-carbonyl)piperidin-4-yl)oxy)-1-methyl-1H-indazol-3-yl)piperidine-2,6-dione C160;
3-(1-methyl-7-((1-(2-(phenylthio) acetyl)piperidin-4-yl)oxy)-1H-indazol-3-yl)-piperidine-2,6-dione C161;
3-(1-methyl-7-((1-(2-(1-methylpiperidin-4-yl)acetyl)piperidin-4-yl)oxy)-1H-indazol-3-yl)piperidine-2,6-dione C162;
6-(4-((3-(2,6-dioxopiperidin-3-yl)-1-methyl-1H-indazol-7-yl)oxy) piperidine-1-carbonyl) nicotinonitrile C163;
3-(7-((1-(cyclobutanecarbonyl)piperidin-4-yl)oxy)-1-methyl-1H-indazol-3-yl)-piperidine-2,6-dione C164;
N-(3-cyclopropyl-1H-pyrazol-5-yl)-2-(3-(2,6-dioxopiperidin-3-yl)-1H-indazol-1-yl)acetamide D001;
2-(3-(2,6-dioxopiperidin-3-yl)-1H-indazol-1-yl)-N-(4-(methylsulfonyl)phenyl)-acetamide D002;
N-(4-(1H-imidazol-5-yl)phenyl)-2-(3-(2,6-dioxopiperidin-3-yl)-1H-indazol-1-yl)-acetamide D003;
2-(3-(2,6-dioxopiperidin-3-yl)-1H-indazol-1-yl)-N-(1-(tetrahydro-2H-pyran-4-yl)-1H-pyrazol-4-yl)acetamide D004;
2-(3-(2,6-dioxopiperidin-3-yl)-1H-indazol-1-yl)-N-(3-ethylisoxazol-5-yl)-acetamide D005;
2-(3-(2,6-dioxopiperidin-3-yl)-1H-indazol-1-yl)-N-(1-methyl-3-(trifluoromethyl)-1H-pyrazol-4-yl)acetamide D006;
2-(3-(2,6-dioxopiperidin-3-yl)-1H-indazol-1-yl)-N-(thiazol-4-yl)acetamide D007;
2-(3-(2,6-dioxopiperidin-3-yl)-1H-indazol-1-yl)-N-(4-(N-methylsulfamoyl)-phenyl)acetamide D008;
2-(3-(2,6-dioxopiperidin-3-yl)-1H-indazol-1-yl)-N-((1-hydroxycyclopropyl)-methyl)acetamide D009;
2-(3-(2,6-dioxopiperidin-3-yl)-1H-indazol-1-yl)-N-(2-methylthiazol-5-yl)-acetamide D010;
2-(3-(2,6-dioxopiperidin-3-yl)-1H-indazol-1-yl)-N-(3-methylisothiazol-5-yl)-acetamide DO11;
2-(3-(2,6-dioxopiperidin-3-yl)-1H-indazol-1-yl)-N-(pyridazin-3-yl)acetamide DO12;
N-(3-cyclopropyl-1-methyl-1H-pyrazol-5-yl)-2-(3-(2,6-dioxopiperidin-3-yl)-1H-indazol-1-yl)acetamide D013;
N-(3,5-dimethylisoxazol-4-yl)-2-(3-(2,6-dioxopiperidin-3-yl)-1H-indazol-1-yl)-acetamide D014;
2-(3-(2,6-dioxopiperidin-3-yl)-1H-indazol-1-yl)-N-(1-isopropyl-1H-pyrazol-4-yl)-acetamide D015;
N-(3-(tert-butyl)-1-phenyl-1H-pyrazol-5-yl)-2-(3-(2,6-dioxopiperidin-3-yl)-1H-indazol-1-yl)acetamide D016;
2-(3-(2,6-dioxopiperidin-3-yl)-1H-indazol-1-yl)-N-(3-methylisoxazol-5-yl)-acetamide D017;
2-(3-(2,6-dioxopiperidin-3-yl)-1H-indazol-1-yl)-N-(4-(oxazol-5-yl)phenyl)-acetamide D018;
N-(1-(tert-butyl)-3-methyl-1H-pyrazol-5-yl)-2-(3-(2,6-dioxopiperidin-3-yl)-1H-indazol-1-yl)acetamide D019;
N-(3,5-dimethyl-1H-pyrazol-4-yl)-2-(3-(2,6-dioxopiperidin-3-yl)-1H-indazol-1-yl)acetamide D020;
2-(3-(2,6-dioxopiperidin-3-yl)-1H-indazol-1-yl)-N-(3-methylisothiazol-4-yl)-acetamide DO21;
2-(3-(2,6-dioxopiperidin-3-yl)-1H-indazol-1-yl)-N-(4-(2-methyl-1H-imidazol-1-yl)phenyl)acetamide D022;
2-(3-(2,6-dioxopiperidin-3-yl)-1H-indazol-1-yl)-N-(4-(4-methylpiperazin-1-yl)-phenyl)acetamide D023;
2-(3-(2,6-dioxopiperidin-3-yl)-1H-indazol-1-yl)-N-(1-methyl-1H-pyrazol-5-yl)-acetamide DO24;
N-(4-(1H-imidazol-2-yl)phenyl)-2-(3-(2,6-dioxopiperidin-3-yl)-1H-indazol-1-yl)-acetamide D025;
N-(3-(1H-imidazol-1-yl)phenyl)-2-(3-(2,6-dioxopiperidin-3-yl)-1H-indazol-1-yl)-acetamide D026;
2-(3-(2,6-dioxopiperidin-3-yl)-1H-indazol-1-yl)-N-((1-methyl-1H-pyrazol-3-yl)-methyl)acetamide D027;
2-(3-(2,6-dioxopiperidin-3-yl)-1H-indazol-1-yl)-N-((1-methyl-1H-imidazol-5-yl)-methyl)acetamide D028;
2-(3-(2,6-dioxopiperidin-3-yl)-1H-indazol-1-yl)-N-(1-methylpiperidin-4-yl)-acetamide D029;
2-(3-(2,6-dioxopiperidin-3-yl)-1H-indazol-1-yl)-N-(3-hydroxycyclopentyl)-acetamide DO30;
N-(1,1-dioxidothietan-3-yl)-2-(3-(2,6-dioxopiperidin-3-yl)-1H-indazol-1-yl)-acetamide D031;
N-(3-(tert-butyl)phenyl)-2-(3-(2,6-dioxopiperidin-3-yl)-1H-indazol-1-yl)-acetamide DO32;
N-(2-(benzyloxy)ethyl)-2-(3-(2,6-dioxopiperidin-3-yl)-1H-indazol-1-yl)-acetamide D033;
2-(3-(2,6-dioxopiperidin-3-yl)-1H-indazol-1-yl)-N-(3-(oxazol-5-yl)phenyl)-acetamide D034;
2-(3-(2,6-dioxopiperidin-3-yl)-1H-indazol-1-yl)-N-(3-isopropoxyphenyl)-acetamide D035;
2-(3-(2,6-dioxopiperidin-3-yl)-1H-indazol-1-yl)-N-(4-(trifluoromethoxy)phenyl)-acetamide D036;
2-(3-(2,6-dioxopiperidin-3-yl)-1H-indazol-1-yl)-N-(4-isopropylbenzyl)acetamide D037;
2-(3-(2,6-dioxopiperidin-3-yl)-1H-indazol-1-yl)-N-(4-hydroxybenzyl)acetamide D038;
2-(3-(2,6-dioxopiperidin-3-yl)-1H-indazol-1-yl)-N-(4-methyltetrahydro-2H-pyran-4-yl)acetamide D039;
N-cyclohexyl-2-(3-(2,6-dioxopiperidin-3-yl)-1H-indazol-1-yl)acetamide D040;
2-(3-(2,6-dioxopiperidin-3-yl)-1H-indazol-1-yl)-N-(thiophen-2-ylmethyl)-acetamide D041;
2-(3-(2,6-dioxopiperidin-3-yl)-1H-indazol-1-yl)-N-((3-hydroxycyclopentyl)-methyl)acetamide D042;
2-(3-(2,6-dioxopiperidin-3-yl)-1H-indazol-1-yl)-N-(tetrahydro-2H-pyran-4-yl)-acetamide D043;
N-(2-(cyclopentyloxy)ethyl)-2-(3-(2,6-dioxopiperidin-3-yl)-1H-indazol-1-yl)-acetamide D044;
2-(3-(2,6-dioxopiperidin-3-yl)-1H-indazol-1-yl)-N-(3-fluorobicyclo[1.1.1]pentan-1-yl)acetamide D045;
2-(3-(2,6-dioxopiperidin-3-yl)-1H-indazol-1-yl)-N-(4-(p-tolyloxy)phenyl)-acetamide D046;
2-(3-(2,6-dioxopiperidin-3-yl)-1H-indazol-1-yl)-N-(thiazol-2-ylmethyl)acetamide D047;
N-(cyclopropylmethyl)-2-(3-(2,6-dioxopiperidin-3-yl)-1H-indazol-1-yl)-acetamide D048;
N-cyclopropyl-2-(3-(2,6-dioxopiperidin-3-yl)-1H-indazol-1-yl)acetamide D049;
2-(3-(2,6-dioxopiperidin-3-yl)-1H-indazol-1-yl)-N-(1-methylazetidin-3-yl)-acetamide D050;
N-(cyclopentylmethyl)-2-(3-(2,6-dioxopiperidin-3-yl)-1H-indazol-1-yl)acetamide D051;
2-(3-(2,6-dioxopiperidin-3-yl)-1H-indazol-1-yl)-N-neopentylacetamide D052;
2-(3-(2,6-dioxopiperidin-3-yl)-1H-indazol-1-yl)-N-(tetrahydrofuran-3-yl)-acetamide D053;
N-cyclopentyl-2-(3-(2,6-dioxopiperidin-3-yl)-1H-indazol-1-yl)acetamide D054;
or 2-(3-(2,6-dioxopiperidin-3-yl)-1H-indazol-1-yl)-N-(2-(methylamino)-2-oxo-ethyl)acetamide D055;
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

67. A pharmaceutical composition comprising the compound of claim 1, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; and a pharmaceutically acceptable excipient.

68-71. (canceled)

72. A method of treating, preventing, or ameliorating one or more symptoms of a proliferative disease in a subject, comprising administering to the subject in need thereof a therapeutically effective amount of a compound of claim 1.
